(19) United States
(12) Patent Application Publication
(10) Pub. No.: US 2025/0243491 A1
(43) Pub. Date: Jul. 31, 2025

(54) PNPLA3-TARGETING SHORT INTERFERING RNA (SIRNA) MOLECULES AND USES THEREOF

(71) Applicants: Aligos Therapeutics, Inc., South San Francisco, CA (US); Merck Sharp & Dohme LLC, Rahway, NJ (US)

(72) Inventors: Leonid BEIGELMAN, South San Francisco, CA (US); Xuan LUONG, South San Francisco, CA (US); Saul MARTINEZ MONTERO, South San Francisco, CA (US); Aneerban BHATTACHARYA, South San Francisco, CA (US); Jerome DEVAL, South San Francisco, CA (US)

(73) Assignees: Aligos Therapeutics, Inc., South San Francisco, CA (US); Merck Sharp & Dohme LLC, Rahway, NJ (US)

(21) Appl. No.: 18/687,072

(22) PCT Filed: Sep. 1, 2022
(86) PCT No.: PCT/US2022/075866

§ 371 Date: Feb. 27, 2024

Related U.S. Application Data
(60) Provisional application No. 63/239,769, filed on Sep. 1, 2021.

(51) Int. Cl. C12N 15/113; A61K 45/06; A61K 47/54

(52) U.S. Cl. CPC C12N 15/1137; A61K 45/06; A61K 47/549; A61K 47/555; C12N 2310/14; C12N 2310/312; C12N 2310/316; C12N 2310/321; C12N 2310/322; C12N 2310/351

(57) ABSTRACT

Disclosed herein are short interfering RNA (siRNA) molecules that downregulate expression of PNPLA3 or variants thereof. The siRNA molecules comprise modified nucleotides and uses thereof. The siRNA molecules may be double stranded and comprise modified nucleotides, such as 2′-O-methyl nucleotides and 2′-fluoro nucleotides, and ligands.

This application claims the priority of U.S. Provisional Patent Application No. U.S. 63/239,769, entitled “PNPLA3-TARGETING SHORT INTERFERING RNA (SIRNA) MOLECULES AND USES THEREOF”, filed Sep. 1, 2021, which is incorporated herein by reference in its entirety for all purposes.

### FIELD OF THE DISCLOSURE

The present disclosure relates to certain PNPLA3-targeting short interfering ribonucleic acid (siRNA) molecules comprising modified nucleotides as well as pharmaceutical compositions comprising the siRNA molecules and uses thereof in the treatment of liver disease.

### BACKGROUND

In parallel with the global increase in obesity, nonalcoholic fatty liver disease (NAFLD) is becoming a leading cause of chronic liver disease and liver transplantation worldwide. NAFLD is a spectrum of chronic liver disorders and is believed to affect about 30% of the adult population and about 70-80% of individuals who are obese and diabetic. NAFLD is generally defined as excess liver fat accumulation greater than 5% induced by causes other than alcohol intake. In a subset of individuals, NAFLD progresses to liver inflammation (nonalcoholic steatohepatitis, NASH), which is associated with fibrosis (scarring of the liver) and may progress to cirrhosis (irreversible advanced liver scarring), which may ultimately lead to liver failure and hepatocellular carcinoma (HCC) in susceptible individuals.

In the United States alone, NASH is the third most common indication for liver transplantation and is on a trajectory to become the most common. The most important medical need in patients with NAFLD and NASH is an effective treatment to halt the progression and possibly reverse fibrosis, which is the main predictor of liver disease evolution.

Unfortunately, therapeutic options for NAFLD and NASH remain limited. The current treatment options focus on weight loss and treatment of secondary conditions, and there are currently no approved pharmaceutical treatments available. Accordingly, there exists a clinical need for improved therapies for the treatment of chronic liver disease, including NAFLD and NASH.

Patatin-like phospholipase domain-containing protein 3 (PNPLA3) is a lipid droplet-associated protein that has hydrolase activity toward triglycerides and retinyl esters. PNPLA3 has been found to be associated with fatty liver disease. Specifically, the PNPLA3 rs738409[G](I148M) variant has been found to be associated with hepatic triglyceride accumulation (steatosis), inflammation, fibrosis, cirrhosis, and hepatocellular carcinoma.

Disclosed herein are siRNA molecules that downregulate expression of PNPLA3 and its variants, pharmaceutical compositions comprising such siRNA molecules, and use of such siRNA molecules and pharmaceutical compositions thereof for treating liver disease and symptoms thereof.

### SUMMARY

One aspect of the present disclosure pertains to a double-stranded short interfering RNA (siRNA) molecule comprising a sense strand comprising a nucleotide sequence that is at least about 60%, 65%, 70%, 75%, 80%, 85%, 90%, 95%, or 100% identical to a nucleotide sequence of any one SEQ ID NOs: 3-452, 903-1484, 2068-2107, 2148-2187, 2228-2252, 2278-2301, 2326-2339 or 2354-2358; and/or an antisense strand comprising a nucleotide sequence that is at least about 60%, 65%, 70%, 75%, 80%, 85%, 90%, 95%, or 100% identical to a nucleotide sequence of any one of SEQ ID NOs: 453-902, 1485-2066, 2108-2147, 2188-2227, 2253-2277, 2302-2325 or 2340-2353, wherein the siRNA molecule downregulates expression of a Patatin-like phospholipase domain-containing protein 3 (PNPLA3) gene.

Another aspect of the present disclosure pertains to a double-stranded short interfering RNA (siRNA) molecule comprising a sense strand comprising a nucleotide sequence of any one SEQ ID NOs: 3-452, 903-1484, 2068-2107, 2148-2187, 2228-2252, 2278-2301, 2326-2339 or 2354-2358; and/or an antisense strand comprising a nucleotide sequence of any one of SEQ ID NOs: 453-902, 1485-2066, 2108-2147, 2188-2227, 2253-2277, 2302-2325 or 2340-2353, wherein the siRNA molecule downregulates expression of a Patatin-like phospholipase domain-containing protein 3 (PNPLA3) gene.

Another aspect of the present disclosure pertains to a double-stranded short interfering RNA (siRNA) molecule selected from any one of siRNA Duplex ID Nos. D1-D515 or MD1-MD673.

Another aspect of the present disclosure pertains to a pharmaceutical composition comprising any of the siRNA molecules according to the disclosure and a pharmaceutically acceptable carrier.

Another aspect of the present disclosure pertains to a method of treating a PNPLA3-associated disease in a subject in need thereof, comprising administering to the subject an amount of any of the siRNA molecules or pharmaceutical compositions according to the disclosure, thereby treating the subject. For example, the liver disease may be NAFLD and/or NASH and/or fatty liver.

Another aspect of the present disclosure pertains to a method of treating a liver disease in a subject in need thereof, comprising administering to the subject an amount of any of the siRNA molecules or pharmaceutical compositions according to the disclosure, thereby treating the subject. For example, the liver disease may be NAFLD and/or NASH and/or fatty liver.

Another aspect of the present disclosure pertains to a method of treating a liver disease in a subject in need thereof, comprising administering to the subject an amount of any of the siRNA molecules or pharmaceutical compositions according to the disclosure, further comprising administering to the subject at least one additional active agent, thereby treating the subject, wherein the at least one additional active agent is a liver disease treatment agent.

Another aspect of the present disclosure pertains to a method of reducing the expression level of PNPLA3 in a patient in need thereof comprising administering to the patient an amount of any of the siRNA molecules or pharmaceutical compositions according to the disclosure, thereby reducing the expression level of PNPLA3 in the patient.

### BRIEF DESCRIPTION OF THE DRAWINGS

FIG. 1 is a diagram of an example of a chemically modified 19-mer siRNA duplex with 2′-F modified nucleotides, 2′-O-methyl (2′-OMe) modified nucleotides, phosphorothioate internucleoside linkages, and UU overhangs.

FIG. 2 is a diagram of an example of a chemically modified 21-mer siRNA duplex with 2′-F modified nucleotides, 2′-OMe modified nucleotides, phosphorothioate internucleoside linkages, UU overhangs, and a vinyl phosphonate on the 5′ end of the antisense strand.

FIG. 3 is a diagram of an example of a chemically modified 19-mer siRNA duplex with four 2′-F modified nucleotides in the sense strand at positions 5, 7, 8, and 9; four 2′-F modified nucleotides in the antisense strand at positions 2, 6, 14, and 16; 2′-OMe modified nucleotides; phosphorothioate internucleoside linkages; a UU overhang; a blunt end; and a possible vinyl phosphonate on the 5′ end of the antisense strand.

FIG. 4 is a diagram of an example of a chemically modified 21-mer siRNA duplex with four 2′-F modified nucleotides in the sense strand at positions 7, 9, 10 and 11; four 2′-F modified nucleotides in the antisense strand at positions 2, 6, 14, and 16; 2′-OMe modified nucleotides; phosphorothioate internucleoside linkages; a UU overhang; a blunt end; and a possible vinyl phosphonate on the 5′ end of the antisense strand.

FIG. 5 is a diagram of an example of a chemically modified 21-mer siRNA duplex with six 2′-F modified nucleotides in the sense strand at positions 5, 9, 10, 11, 14 and 19; two 2′-F modified nucleotides in the antisense strand at positions 2 and 14; 2′-OMe modified nucleotides; phosphorothioate internucleoside linkages; a UU overhang; a blunt end; and a possible vinyl phosphonate on the 5′ end of the antisense strand.

FIG. 6 is a diagram of an example of a chemically modified 19-mer siRNA duplex with four 2′-F modified nucleotides in the sense strand at positions 5, 7, 8, and 9; four 2′-F modified nucleotides in the antisense strand at positions 2, 6, 14, and 16; 2′-OMe modified nucleotides; phosphorothioate internucleoside linkages; a UU overhang; a blunt end; a possible vinyl phosphonate on the 5′ end of the antisense strand; and three monomeric GalNAc4 units (“GalNAc4-ps-GalNAc4-ps GalNAc4”) at the 3′-end of the sense strand.

FIG. 7 is a diagram of an example of a chemically modified 21-mer siRNA duplex with four 2′-F modified nucleotides in the sense strand at positions 7, 9, 10 and 11; four 2′-F modified nucleotides in the antisense strand at positions 2, 6, 14, and 16; 2′-OMe modified nucleotides; phosphorothioate internucleoside linkages; a UU overhang; a blunt end; a possible vinyl phosphonate on the 5′ end of the antisense strand; and three monomeric GalNAc4 units (“GalNAc4-ps-GalNAc4-ps GalNAc4”) at the 3′-end of the sense strand.

FIG. 8 is a diagram of an example of a chemically modified 21-mer siRNA duplex with six 2′-F modified nucleotides in the sense strand at positions 5, 9, 10, 11, 14, and 19; two 2′-F modified nucleotides in the antisense strand at positions 2 and 14; 2′-OMe modified nucleotides; phosphorothioate internucleoside linkages; a UU overhang; a blunt end; a possible vinyl phosphonate on the 5′ end of the antisense strand; and three monomeric GalNAc4 units (“GalNAc4-ps-GalNAc4-ps GalNAc4”) at the 3′-end of the sense strand.

FIG. 9 shows the % of PNPLA3 RNA inhibition in hPNPLA3-KI mice transformed with modified siRNA duplexes according to the present disclosure.

FIG. 10 shows the % of PNPLA3 RNA inhibition in hPNPLA3-KI mice transformed with modified siRNA duplexes according to the present disclosure.

FIG. 11 shows the % of PNPLA3 RNA inhibition in hPNPLA3-KI mice transformed with modified siRNA duplexes according to the present disclosure.

FIG. 12 shows the % of PNPLA3 RNA inhibition in hPNPLA3-KI mice transformed with modified siRNA duplexes according to the present disclosure.

FIG. 13 shows the % of PNPLA3 RNA inhibition in hPNPLA3-KI mice transformed with modified siRNA duplexes according to the present disclosure.

FIG. 14 shows the % of PNPLA3 RNA inhibition in hPNPLA3-KI mice transformed with modified siRNA duplexes according to the present disclosure.

FIG. 15 shows the % of PNPLA3 RNA inhibition in hPNPLA3-KI mice transformed with modified siRNA duplexes according to the present disclosure.

FIG. 16 shows the % of PNPLA3 RNA inhibition in hPNPLA3-KI mice transformed with modified siRNA duplexes according to the present disclosure.

### DETAILED DESCRIPTION

This section presents a detailed description of the many different aspects and embodiments that are representative of the disclosure. This description is by way of several exemplary illustrations of varying detail and specificity. Other features and advantages of these embodiments are apparent from the additional descriptions provided herein, including the different examples. The provided examples illustrate different components and methodology useful in practicing various embodiments of the disclosure. The examples are not intended to limit the claimed disclosure. Based on the present disclosure, the ordinarily skilled artisan can identify and employ other components and methodology useful for practicing the present disclosure.

The present disclosure will be better understood with reference to the following definitions.

### Definitions

Unless defined otherwise, all technical and scientific terms used herein have the meaning commonly understood by a person of ordinary skill in the art to which this disclosure belongs.

The terms “a” and “an” as used herein mean “one or more” and include the plural unless the context is inappropriate.

The term “about” as used herein when referring to a measurable value (e.g., weight, time, and dose) is meant to encompass variations, such as ±10%, 5%, ±1%, or ±0.1% of the specified value.

Except where otherwise indicated, all numbers expressing quantities of ingredients, reaction conditions, and so forth used in the specification and claims are to be understood as being modified in all instances by the term “about,” whether or not the term “about” is present in front of the number. Accordingly, unless indicated to the contrary, the numerical parameters set forth in the following specification and attached claims are approximations that may vary depending upon the desired properties sought to be obtained by the present disclosure. At the very least, and not to be considered as an attempt to limit the application of the doctrine of equivalents to the scope of the claims, each numerical parameter should be construed in light of the number of significant digits and ordinary rounding conventions.

Additionally, the disclosure of numerical ranges within this specification is considered to be a disclosure of all numerical values and ranges within that range. For example, if a range is from about 1 to about 50, it is deemed to include, for example, 1, 50, 7, 34, 46.1, 23.7, or any other value or range within the range. Moreover, as used herein, the term “at least” includes the stated number, e.g., “at least 50” includes 50.

As a general matter, compositions specifying a percentage are specifying a percentage by weight unless otherwise specified. Further, if a variable is not accompanied by a definition, then the previous definition of the variable controls.

The term “including” is used herein to mean, and is used interchangeably with, the phrase “including, but not limited to”.

As used herein, the terms “siRNA” and “siRNA molecule” and “siNA” are used interchangeably and refer to short (or small) interfering ribonucleic acid (RNA), including chemically modified RNA, which may be single-stranded or double-stranded. As used herein, the siRNA may comprise modified nucleotides, including modifications at the sugar, nucleobase, and/or phosphodiester backbone (internucleoside linkage), and nucleoside analogs, as well as conjugates or ligands. As used herein, the term “siRNA duplex” refers to a double-stranded (“ds”) siRNA or “dsRNA” or “ds-NA” having a sense strand and an antisense strand.

As used herein, the term “antisense strand” or “guide strand” refers to the strand of a siRNA molecule which includes a region that is substantially complementary to a target sequence, e.g., a PNPLA3 mRNA.

As used herein, the term “sense strand” or “passenger strand” refers to the strand of a siRNA molecule that includes a region that is substantially complementary to a region of the antisense strand as that term is defined herein.

As used herein, the term “modified nucleotide” refers to a nucleotide having, independently, modifications at the sugar, nucleobase, and/or phosphodiester backbone (internucleoside linkage), and nucleoside analogs. Thus, the term modified nucleotide encompasses substitutions, additions, or removal of, e.g., a functional group or atom, to internucleoside linkages, sugar moieties, or nucleobases. The modifications suitable for use in the siRNAs of the disclosure include all types of modifications disclosed herein or known in the art. Any such modifications, as used in a siRNA molecule, are encompassed by “siRNA” and “siRNA molecule” and “siRNA duplex” for the purposes of this specification and claims. It will also be understood that the term “nucleotide” can also refer to a modified nucleotide, as further detailed herein.

As used herein, the term “nucleobase” refers to naturally-occurring nucleobases and their analogues. Examples of naturally-occurring nucleobases or their analogues include, but are not limited to, thymine, uracil, adenine, cytosine, guanine, aryl, heteroaryl, and an analogue or derivative thereof.

As used herein, the term “nucleotide overhang” or “overhang” refers to at least one unpaired nucleotide that protrudes from the duplex structure of a double-stranded RNA (e.g., siRNA duplex or dsRNA). For example, when a 3′ end of one strand of a dsRNA extends beyond the 5′ end of the other strand, or vice versa, there is a nucleotide overhang. The overhang(s) can be on the sense strand, the antisense strand or any combination thereof. Furthermore, the nucleotide(s) of an overhang can be present on the 5′ end, 3′ end or both ends of an antisense and/or sense strand of a dsRNA and can comprise modified nucleotides. Generally, if any nucleotide overhangs, as defined herein, are present, the sequence of such overhangs is not considered in determining the degree of complementarity between two sequences and such overhangs shall not be regarded as mismatches with regard to the determination of complementarity. By way of example, a sense strand of 21 nucleotides in length and an antisense strand of 21 nucleotides in length that hybridizes to form a 19 base pair duplex region with a 2 nucleotide overhang at the 3′ end of each strand would be considered to be fully complementary as the term is used herein.

As used herein, the term “blunt end” refers to an end of a dsRNA with no unpaired nucleotides, i.e., no nucleotide overhang. In some embodiments, a blunt end can be present on one or both ends of a dsRNA.

The terms “complementary,” “fully complementary” and “substantially complementary” herein can be used with respect to the base pairing between the sense strand and the antisense strand of a duplex siRNA or dsRNA, or between the antisense strand of a siRNA and a target sequence, as will be understood from the context of their use. As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of ordinary skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. In some embodiments, a sequence is “substantially complementary” to a target sequence if the sequence is at least about 80%, 85%, 90%, 95%, 96%, 97%, 98%, 99% or 100% complementary to a target sequence. Percent complementarity can be calculated, for example, by dividing the number of bases in a first sequence that are complementary to bases at corresponding positions in a second or target sequence by the total length of the first sequence. Such calculations are well within the ability of those ordinarily skilled in the art. A sequence may also be said to be substantially complementary to another sequence if there are no more than 5, 4, 3, 2, or 1 mismatches over a 30 base pair duplex region, for example, when the two sequences are hybridized. “Complementary” sequences, as used herein, can also include, or be formed entirely from, non-Watson-Crick base pairs and/or base pairs formed from modified nucleotides, in so far as the above requirements with respect to their ability to hybridize are fulfilled.

The use of percent identity (i.e., “identical”) is a common way of defining the number of differences in the nucleobases between two nucleic acid sequences. For example, where a first sequence is ACGT, a second sequence of ACGA would be considered a “non-identical” sequence with one difference. Percent identity may be calculated over the entire length of a sequence, or over a portion of the sequence. Percent identity may be calculated according to the number of nucleobases that have identical base pairing corresponding to the sequence to which it is being compared. The non-identical nucleobases may be adjacent to each other, dispersed throughout the sequence, or both. Such calculations are well within the ability of those ordinarily skilled in the art.

As used herein, “missense mutation” refers to when a change in a single base pair results in a substitution of a different amino acid in the resulting protein.

As used herein, the term “effective amount” or “therapeutically effective amount” refers to the amount of a siRNA of the present disclosure sufficient to effect beneficial or desired results, such as for example, the amount that will elicit the biological or medical response of a tissue, system, animal, or human that is being sought by a researcher, veterinarian, medical doctor, or other clinician. A therapeutically effective amount can be administered in one or more administrations, applications, or dosages and is not intended to be limited to a particular formulation or administration route. In some embodiments, “therapeutically effective amount” means an amount that alleviates at least one clinical symptom in a human patient, e.g., at least one symptom of a PNPLA3-associated disease or a liver disease.

As used herein, the terms “patient” and “subject” refer to organisms who use the siRNA molecules of the disclosure for the prevention or treatment of a medical condition, including in the methods of the present disclosure. Such organisms are preferably mammals, and more preferably humans. As used herein, a subject “in need” of treatment of an existing condition or of prophylactic treatment encompasses both a determination of need by a medical professional as well as the desire of a patient for such treatment. Administering of the compound (e.g., a siRNA of the present disclosure) to the subject includes both self-administration and administration to the patient by another.

As used herein, the term “active agent” or “active ingredient” or “therapeutic agent” refers to an ingredient with a pharmacological effect, such as a therapeutic effect, at a relevant dose. This includes siRNA molecules according to the disclosure.

As used herein, a “liver disease treatment agent” is an active agent which can be used to treat liver disease, either alone or in combination with another active agent, and is other than the siRNA of the present disclosure.

As used herein, the term “pharmaceutical composition” refers to the combination of at least one active agent with a carrier, inert or active, making the composition especially suitable for diagnostic or therapeutic use in vivo or ex vivo. In some embodiments, the term “pharmaceutical composition” means a composition comprising a siRNA molecule as described herein and at least one additional component selected from pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the mode of administration and dosage form used.

As used herein, the term “pharmaceutically acceptable carrier” refers to any pharmaceutical carrier, diluent, adjuvant, excipient, or vehicle, including those described herein, for example, solvents, buffers, solutions (e.g., a phosphate buffered saline solution), water, emulsions (e.g., such as an oil/water or water/oil emulsions), various types of wetting agents, stabilizers, preservatives, antibacterial and antifungal agents, dispersion media, coatings, isotonic and absorption delaying agents and the like acceptable for use in formulating pharmaceuticals, including, for example, pharmaceuticals suitable for administration to humans. For examples of carriers, see, for example, Martin, Remington's Pharmaceutical Sciences, 15th Ed., Mack Publ. Co., Easton, PA [1975].

As used herein, the terms “treat”, “treating”, and “treatment” include any effect, e.g., lessening, reducing, modulating, ameliorating or eliminating, that results in the improvement of the condition, disease, disorder, and the like; or of one or more symptoms associated with the condition, disease, or disorder; or of the cause(s) of the condition, disease, or disorder. For example, with respect to PNPLA-associated disease, the terms “treat”, “treating”, and “treatment” include, but are not limited to, alleviation or amelioration of one or more symptoms associated with PNPLA3 gene expression and/or PNPLA3 protein production, e.g., the presence of increased protein activity in the hedgehog (Hh) signaling pathway, fatty liver (steatosis), nonalcoholic steatohepatitis (NASH), cirrhosis of the liver, accumulation of fat in the liver, inflammation of the liver, hepatocellular necrosis, liver fibrosis, obesity, or nonalcoholic fatty liver disease (NAFLD). “Treatment” can also mean prolonging survival as compared to expected survival in the absence of treatment.

As used herein, the terms “alleviate” and “alleviating” refer to reducing the severity of the condition and/or a symptom thereof, such as reducing the severity by, for example, at least 10%, 20%, 30%, 40%, 50%, 60%, 70%, 80%, 90%, or 95%.

As used herein, the term “downregulate” or “downregulating” is used interchangeably with “reducing”, “inhibiting”, or “suppressing” or other similar terms, and includes any level of downregulation.

As used herein, the term “PNPLA3 gene” refers to the Patatin-like phospholipase domain-containing protein 3 gene and includes variants thereof. The sequence for the human wild-type PNPLA3 gene may be found in, for example, NCBI Ref. No. NM_025225.3 and SEQ ID NO: 1. Additional examples of PNPLA3 gene sequences, including for other mammalian genes, are readily available using public databases, including, for example, NCBI RefSeq, GenBank, UniProt, and OMIM.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

siRNA Molecules

Disclosed herein are double-stranded short (or small) interfering RNA (siRNA) molecules that specifically downregulate expression of a Patatin-like phospholipase domain-containing protein 3 (PNPLA3) gene.

In some embodiments, the double-stranded siRNA molecule comprises (a) a sense strand comprising a nucleotide sequence that is at least about 60%, 65%, 70%, 75%, 80%, 85%, 90%, 95%, or 100% identical to the nucleotide sequence of any one of SEQ ID NOs: 3-452, 903-1484, 2068-2107, 2148-2187, 2228-2252, 2278-2301, 2326-2339 or 2354-2358; and/or (b) an antisense strand comprising a nucleotide sequence that is at least about 60%, 65%, 70%, 75%, 80%, 85%, 90%, 95%, or 100% identical to the nucleotide sequence of any one of SEQ ID NOs: 453-902, 1485-2066, 2108-2147, 2188-2227, 2253-2277, 2302-2325 or 2340-2353.

In some embodiments, the double-stranded siRNA molecule comprises (a) a sense strand comprising a nucleotide sequence of any one SEQ ID NOs: 3-452, 903-1484, 2068-2107, 2148-2187, 2228-2252, 2278-2301, 2326-2339 or 2354-2358; and/or (b) an antisense strand comprising a nucleotide sequence of any one of SEQ ID NOs: 453-902, 1485-2066, 2108-2147, 2188-2227, 2253-2277, 2302-2325 or 2340-2353.

In some embodiments, the double-stranded siRNA molecule comprises a sense strand comprising a nucleotide sequence of any one of SEQ ID NOs: 3-452. In some embodiments, the siRNA molecule comprises an antisense strand comprising a nucleotide sequence of any one of SEQ ID NOs: 453-902. In some embodiments, the siRNA molecule comprises (a) a sense strand comprising a nucleotide sequence of any one of SEQ ID NOs: 3-452 and (b) an antisense strand comprising a nucleotide sequence of any one of SEQ ID NOs: 453-902.

In some embodiments, the double-stranded siRNA molecule comprises a sense strand comprising a nucleotide sequence of any one of SEQ ID NOs: 903-1484. In some embodiments, the siRNA molecule comprises an antisense strand comprising a nucleotide sequence of any one of SEQ ID NOs: 1485-2066. In some embodiments, the siRNA molecule comprises (a) a sense strand comprising a nucleotide sequence of any one of SEQ ID NOs: 903-1484 and (b) an antisense strand comprising a nucleotide sequence of any one of SEQ ID NOs: 1485-2066.

In some embodiments, the double-stranded siRNA molecule comprises a sense strand comprising a nucleotide sequence of any one of SEQ ID NOs: 2068-2107. In some embodiments, the siRNA molecule comprises an antisense strand comprising a nucleotide sequence of any one of SEQ ID NOs: 2108-2147. In some embodiments, the siRNA molecule comprises (a) a sense strand comprising a nucleotide sequence of any one of SEQ ID NOs: 2068-2107 and (b) an antisense strand comprising a nucleotide sequence of any one of SEQ ID NOs: 2108-2147.

In some embodiments, the double-stranded siRNA molecule comprises a sense strand comprising a nucleotide sequence of any one of SEQ ID NOs: 2148-2187. In some embodiments, the siRNA molecule comprises an antisense strand comprising a nucleotide sequence of any one of SEQ ID NOs: 2188-2227. In some embodiments, the siRNA molecule comprises (a) a sense strand comprising a nucleotide sequence of any one of SEQ ID NOs: 2148-2187 and (b) an antisense strand comprising a nucleotide sequence of any one of SEQ ID NOs: 2188-2227.

In some embodiments, the double-stranded siRNA molecule comprises a sense strand comprising a nucleotide sequence of any one of SEQ ID NOs: 2228-2252. In some embodiments, the siRNA molecule comprises an antisense strand comprising a nucleotide sequence of any one of SEQ ID NOs: 2253-2277. In some embodiments, the siRNA molecule comprises (a) a sense strand comprising a nucleotide sequence of any one of SEQ ID NOs: 2228-2252 and (b) an antisense strand comprising a nucleotide sequence of any one of SEQ ID NOs: 2253-2277.

In some embodiments, the double-stranded siRNA molecule comprises a sense strand comprising a nucleotide sequence of any one of SEQ ID NOs: 2278-2301, 2326-2339 or 2354-2358. In some embodiments, the siRNA molecule comprises an antisense strand comprising a nucleotide sequence of any one of SEQ ID NOs: 2302-2325 or 2340-2353. In some embodiments, the siRNA molecule comprises (a) a sense strand comprising a nucleotide sequence of any one of SEQ ID NOs: 2278-2301, 2326-2339 or 2354-2358 and (b) an antisense strand comprising a nucleotide sequence of any one of SEQ ID NOs: 2302-2325 or 2340-2353.

In some embodiments, the double-stranded siRNA molecule comprises (a) a sense strand comprising at least about 15, 16, 17, 18, 19, 20, or 21 consecutive nucleotides of the nucleotide sequence of any one SEQ ID NOs: 3-452, 903-1484, 2068-2107, 2148-2187, 2228-2252, 2278-2301, 2326-2339 or 2354-2358; and/or (b) an antisense strand comprising at least about 15, 16, 17, 18, 19, 20, 21, 22, or 23 consecutive nucleotides of the nucleotide sequence of any one of SEQ ID NOs: 453-902, 1485-2066, 2108-2147, 2188-2227, 2253-2277, 2302-2325 or 2340-2353.

In some embodiments, the double-stranded siRNA molecule comprises (a) a sense strand comprising a nucleotide sequence having at least about 15, 16, 17, 18, 19, 20, or 21 consecutive nucleotides of the nucleotide sequence of any one of SEQ ID NOs: 3-452, 903-1484, 2068-2107, 2148-2187, 2228-2252, 2278-2301, 2326-2339 or 2354-2358; and/or (b) an antisense strand comprising a nucleotide sequence having at least about 15, 16, 17, 18, 19, 20, 21, 22, or 23 consecutive nucleotides of the nucleotide sequence of any one of SEQ ID NOs: 453-902, 1485-2066, 2108-2147, 2188-2227, 2253-2277, 2302-2325 or 2340-2353.

In some embodiments, at least one end of the double-stranded siRNA molecule is a blunt end. In some embodiments, both ends of the double-stranded siRNA molecule are blunt ends. In some embodiments, one end of the double-stranded siRNA molecule comprises a blunt end and one end of the double-stranded siRNA molecule comprises an overhang.

In some embodiments, at least one end of the siRNA molecule comprises an overhang, wherein the overhang comprises at least one unpaired nucleotide. In some embodiments, at least one end of the siRNA molecule comprises an overhang, wherein the overhang comprises at least two unpaired nucleotides. In some embodiments, both ends of the siRNA molecule comprise an overhang, wherein the overhang comprises at least one unpaired nucleotide. In some embodiments, both ends of the siRNA molecule comprise an overhang, wherein the overhang comprises at least two unpaired nucleotides. In some embodiments, the siRNA molecule comprises an overhang of two unpaired nucleotides at the 3′ end of the sense strand. In some embodiments, the siRNA molecule comprises an overhang of two unpaired nucleotides at the 3′ end of the antisense strand. In some embodiments, the siRNA molecule comprises an overhang of two unpaired nucleotides at the 3′ end of the sense strand and the 3′ end of the antisense strand.

In some embodiments, the double stranded siRNA molecule is selected from any one of siRNA Duplex ID Nos. D1-D515 or MD1-MD-687. In some embodiments, the double stranded siRNA molecule is selected from any one of siRNA Duplex ID Nos. D1-D515. In some embodiments, the double stranded siRNA molecule is selected from any one of siRNA Duplex ID Nos. MD1-MD687.

In some embodiments, the double stranded siRNA molecule is selected from any one of the siRNA Duplexes of Table 1 or Table 1A or Table 2. In some embodiments, the double stranded siRNA molecule is selected from any one of the siRNA Duplexes of Table 1. In some embodiments, the double stranded siRNA molecule is selected from any one of the siRNA Duplexes of Table 1A. In some embodiments, the double stranded siRNA molecule is selected from any one of the siRNA Duplexes of Table 2.

In some embodiments, the double stranded siRNA molecule is about 17 to about 29 base pairs in length, or from 19-23 base pairs, or from 19-21 base pairs, one strand of which is complementary to a target mRNA, that when added to a cell having the target mRNA, or produced in the cell in vivo, causes degradation of the target mRNA.

In some embodiments, the siRNA molecules of the disclosure comprise a nucleotide sequence that is complementary to a nucleotide sequence of a target gene. In some embodiments, the siRNA molecule of the disclosure interacts with a nucleotide sequence of a target gene in a manner that causes inhibition of expression of the target gene.

The siRNA molecules can be obtained using any one of a number of techniques known to those of ordinary skill in the art. In some embodiments, the siRNA molecules may be synthesized as two separate, complementary nucleic acid molecules, or as a single nucleic acid molecule with two complementary regions. For example, the siRNAs of the disclosure may be chemically synthesized using appropriately protected ribonucleoside phosphoramidites and a conventional RNA synthesizer or other well-known methods. In addition, the siRNAs may be produced by a commercial supplier, such as, for example, Dharmacon/Horizon (Lafayette, Colo., USA), Glen Research (Sterling, Va., USA), ChemGenes (Ashland, Mass., USA) and Cruachem (Glasgow, UK). In some embodiments, the siRNA molecules may be encoded by a plasmid.

### Sense Strand

Any of the siRNA molecules described herein may comprise a sense strand. In some embodiments, the sense strand comprises between about 15 to about 50 nucleotides. In some embodiments, the sense strand comprises between about 15 to about 45 nucleotides. In some embodiments, the sense strand comprises between about 15 to about 40 nucleotides. In some embodiments, the sense strand comprises between about 15 to about 35 nucleotides. In some embodiments, the sense strand comprises between about 15 to about 30 nucleotides. In some embodiments, the sense strand comprises between about 15 to about 25 nucleotides. In some embodiments, the sense strand comprises between about 17 to about 23 nucleotides. In some embodiments, the sense strand comprises between about 17 to about 22 nucleotides. In some embodiments, the sense strand comprises between about 17 to about 21 nucleotides. In some embodiments, the sense strand comprises between about 18 to about 23 nucleotides. In some embodiments, the sense strand comprises between about 18 to about 22 nucleotides. In some embodiments, the sense strand comprises between about 18 to about 21 nucleotides. In some embodiments, the sense strand comprises between about 19 to about 23 nucleotides. In some embodiments, the sense strand comprises between about 19 to about 22 nucleotides. In some embodiments, the sense strand comprises between about 19 to about 21 nucleotides.

In some embodiments, the sense strand comprises at least about 15, 16, 17, 18, 19, 20, 21, 22, 23, 24, 25, 26, 27, 28, 29, or 30 or more nucleotides. In some embodiments, the sense strand comprises at least about 15 nucleotides. In some embodiments, the sense strand comprises at least about 16 nucleotides. In some embodiments, the sense strand comprises at least about 17 nucleotides. In some embodiments, the sense strand comprises at least about 18 nucleotides. In some embodiments, the sense strand comprises at least about 19 nucleotides. In some embodiments, the sense strand comprises at least about 20 nucleotides. In some embodiments, the sense strand comprises at least about 21 nucleotides. In some embodiments, the sense strand comprises at least about 22 nucleotides. In some embodiments, the sense strand comprises at least about 23 nucleotides.

In some embodiments, the sense strand comprises less than about 50, 45, 40, 35, 30, 29, 28, 27, 26, 25, 24, 23, 22, 21, 20, or 19 or fewer nucleotides. In some embodiments, the sense strand comprises less than about 30 nucleotides. In some embodiments, the sense strand comprises less than about 25 nucleotides. In some embodiments, the sense strand comprises less than about 24 nucleotides. In some embodiments, the sense strand comprises less than about 23 nucleotides. In some embodiments, the sense strand comprises less than about 22 nucleotides. In some embodiments, the sense strand comprises less than about 21 nucleotides. In some embodiments, the sense strand comprises less than about 20 nucleotides. In some embodiments, the sense strand comprises less than about 19 nucleotides.

In some embodiments, the sense strand comprises a sequence that is at least about 60%, 65%, 70%, 75%, 80%, 85%, 90%, 95%, or 100% identical to a fragment of the PNPLA3 gene across the entire length of the sense strand. In some embodiments, the sense strand comprises a sequence that is at least about 70% identical to a fragment of the PNPLA3 gene across the entire length of the sense strand. In some embodiments, the sense strand comprises a sequence that is at least about 75% identical to a fragment of the PNPLA3 gene across the entire length of the sense strand. In some embodiments, the sense strand comprises a sequence that is at least about 80% identical to a fragment of the PNPLA3 gene across the entire length of the sense strand. In some embodiments, the sense strand comprises a sequence that is at least about 85% identical to a fragment of the PNPLA3 gene across the entire length of the sense strand. In some embodiments, the sense strand comprises a sequence that is at least about 90% identical to a fragment of the PNPLA3 gene across the entire length of the sense strand. In some embodiments, the sense strand comprises a sequence that is at least about 95% identical to a fragment of the PNPLA3 gene across the entire length of the sense strand. In some embodiments, the sense strand comprises a sequence that is about 100% identical to a fragment of the PNPLA3 gene across the entire length of the sense strand. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the sense strand comprises a sequence having between about 15 to about 50 consecutive nucleotides of a fragment of the PNPLA3 gene. In some embodiments, the sense strand comprises a sequence having between about 15 to about 45 consecutive nucleotides of a fragment of the PNPLA3 gene. In some embodiments, the sense strand comprises a sequence having between about 15 to about 40 consecutive nucleotides of a fragment of the PNPLA3 gene. In some embodiments, the sense strand comprises a sequence having between about 15 to about 35 consecutive nucleotides of a fragment of the PNPLA3 gene. In some embodiments, the sense strand comprises a sequence having between about 15 to about 30 consecutive nucleotides of a fragment of the PNPLA3 gene. In some embodiments, the sense strand comprises a sequence having between about 15 to about 25 consecutive nucleotides of a fragment of the PNPLA3 gene. In some embodiments, the sense strand comprises between about 17 to about 23 consecutive nucleotides of a fragment of the PNPLA3 gene. In some embodiments, the sense strand comprises between about 17 to about 22 consecutive nucleotides of a fragment of the PNPLA3 gene. In some embodiments, the sense strand comprises between about 17 to about 21 consecutive nucleotides of a fragment of the PNPLA3 gene. In some embodiments, the sense strand comprises between about 18 to about 23 consecutive nucleotides of a fragment of the PNPLA3 gene. In some embodiments, the sense strand comprises between about 18 to about 22 consecutive nucleotides of a fragment of the PNPLA3 gene. In some embodiments, the sense strand comprises between about 18 to about 21 consecutive nucleotides of a fragment of the PNPLA3 gene. In some embodiments, the sense strand comprises between about 19 to about 23 consecutive nucleotides of a fragment of the PNPLA3 gene. In some embodiments, the sense strand comprises between about 19 to about 22 consecutive nucleotides of a fragment of the PNPLA3 gene. In some embodiments, the sense strand comprises between about 19 to about 21 consecutive nucleotides of a fragment of the PNPLA3 gene. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the sense strand comprises a sequence having at least about 15, 16, 17, 18, 19, 20, 21, 22, 23, 24, 25, 26, 27, 28, 29, or 30 or more consecutive nucleotides of a fragment of the PNPLA3 gene. In some embodiments, the sense strand comprises a sequence having at least about 15 consecutive nucleotides of a fragment of the PNPLA3 gene. In some embodiments, the sense strand comprises a sequence having at least about 16 consecutive nucleotides of a fragment of the PNPLA3 gene. In some embodiments, the sense strand comprises a sequence having at least about 17 consecutive nucleotides of a fragment of the PNPLA3 gene. In some embodiments, the sense strand comprises a sequence having at least about 18 consecutive nucleotides of a fragment of the PNPLA3 gene. In some embodiments, the sense strand comprises a sequence having at least about 19 consecutive nucleotides of a fragment of the PNPLA3 gene. In some embodiments, the sense strand comprises a sequence having at least about 20 consecutive nucleotides of a fragment of the PNPLA3 gene. In some embodiments, the sense strand comprises a sequence having at least about 21 consecutive nucleotides of a fragment of the PNPLA3 gene. In some embodiments, the sense strand comprises a sequence having at least about 22 consecutive nucleotides of a fragment of the PNPLA3 gene. In some embodiments, the sense strand comprises a sequence having at least about 23 consecutive nucleotides of a fragment of the PNPLA3 gene. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the sense strand comprises a sequence having less than about 50, 45, 40, 35, 30, 29, 28, 27, 26, 25, 24, 23, 22, 21, 20, or 19 or fewer consecutive nucleotides of a fragment of the PNPLA3 gene. In some embodiments, the sense strand comprises a sequence having less than about 35 consecutive nucleotides of a fragment of the PNPLA3 gene. In some embodiments, the sense strand comprises a sequence having less than about 30 consecutive nucleotides of a fragment of the PNPLA3 gene. In some embodiments, the sense strand comprises a sequence having less than about 25 consecutive nucleotides of a fragment of the PNPLA3 gene. In some embodiments, the sense strand comprises a sequence having less than about 24 consecutive nucleotides of a fragment of the PNPLA3 gene. In some embodiments, the sense strand comprises a sequence having less than about 23 consecutive nucleotides of a fragment of the PNPLA3 gene. In some embodiments, the sense strand comprises a sequence having less than about 22 consecutive nucleotides of a fragment of the PNPLA3 gene. In some embodiments, the sense strand comprises a sequence having less than about 21 consecutive nucleotides of a fragment of the PNPLA3 gene. In some embodiments, the sense strand comprises a sequence having less than about 20 consecutive nucleotides of a fragment of the PNPLA3 gene. In some embodiments, the sense strand comprises a sequence having less than about 19 consecutive nucleotides of a fragment of the PNPLA3 gene. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the sense strand comprises a sequence having less than or equal to 5, 4, 3, 2, or 1 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the sense strand comprises a sequence having less than or equal to 5 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the sense strand comprises a sequence having less than or equal to 4 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the sense strand comprises a sequence having less than or equal to 3 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the sense strand comprises a sequence having less than or equal to 2 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the sense strand comprises a sequence having less than or equal to 1 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the sense strand comprises a sequence having 0 nucleobase differences to a fragment of the PNPLA3 gene across the entire length of the sense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the sense strand comprises a nucleotide sequence of any one of SEQ ID NOs: 3-452, 903-1484, 2068-2107, 2148-2187, 2228-2252, 2278-2301, 2326-2339 or 2354-2358. In some embodiments, the sense strand comprises a nucleotide sequence that is at least about 60%, 65%, 70%, 75%, 80%, 85%, 90%, 95%, or 100% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is at least about 70% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is at least about 75% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is at least about 80% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is at least about 85% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is at least about 90% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is at least about 95% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand. In some embodiments, the sense strand comprises a nucleotide sequence that is about 100% identical to the nucleotide sequence of any one of SEQ ID NOs: 3-452, 903-1484, 2068-2107, 2148-2187, 2228-2252, 2278-2301, 2326-2339 or 2354-2358 across the entire length of sense strand.

In some embodiments, the sense strand comprises at least about 15, 16, 17, 18, 19, 20, or 21 consecutive nucleotides of the nucleotide sequence of any one of SEQ ID NOs: 3-452, 903-1484, 2068-2107, 2148-2187, 2228-2252, 2278-2301, 2326-2339 or 2354-2358. In some embodiments, the sense strand comprises at least about 17 consecutive nucleotides of the nucleotide sequence of any one of SEQ ID NOs: 3-452, 903-1484, 2068-2107, 2148-2187, 2228-2252, 2278-2301, 2326-2339 or 2354-2358. In some embodiments, the sense strand comprises at least about 18 consecutive nucleotides of the nucleotide sequence of any one of SEQ ID NOs: 3-452, 903-1484, 2068-2107, 2148-2187, 2228-2252, 2278-2301, 2326-2339 or 2354-2358. In some embodiments, the sense strand comprises at least about 19 consecutive nucleotides of the nucleotide sequence of any one of SEQ ID NOs: 3-452, 903-1484, 2068-2107, 2148-2187, 2228-2252, 2278-2301, 2326-2339 or 2354-2358. In some embodiments, the sense strand comprises at least about 20 consecutive nucleotides of the nucleotide sequence of any one of SEQ ID NOs: 3-452, 903-1484, 2068-2107, 2148-2187, 2228-2252, 2278-2301, 2326-2339 or 2354-2358. In some embodiments, the sense strand comprises at least about 21 consecutive nucleotides of the nucleotide sequence of any one of SEQ ID NOs: 3-452, 903-1484, 2068-2107, 2148-2187, 2228-2252, 2278-2301, 2326-2339 or 2354-2358.

In some embodiments, the sense strand comprises a nucleotide sequence having less than or equal to 5, 4, 3, 2, or 1 mismatches to the nucleotide sequence of any one of SEQ ID NOs: 453-902 or 1485-2066 across the entire length of the sense strand. In some embodiments, the sense strand comprises a nucleotide sequence having less than or equal to 5 nucleobase differences to the nucleotide sequence of any one of SEQ ID NOs: 453-902, 1485-2066, 2108-2147, 2188-2227, 2253-2277, 2302-2325 or 2340-2353 across the entire length of the sense strand. In some embodiments, the sense strand comprises a nucleotide sequence having less than or equal to 4 nucleobase differences to the nucleotide sequence of any one of SEQ ID NOs: 453-902, 1485-2066, 2108-2147, 2188-2227, 2253-2277, 2302-2325 or 2340-2353 across the entire length of the sense strand. In some embodiments, the sense strand comprises a nucleotide sequence having less than or equal to 3 nucleobase differences to the nucleotide sequence of any one of SEQ ID NOs: 453-902, 1485-2066, 2108-2147, 2188-2227, 2253-2277, 2302-2325 or 2340-2353 across the entire length of the sense strand. In some embodiments, the sense strand comprises a nucleotide sequence having less than or equal to 2 nucleobase differences to the nucleotide sequence of any one of SEQ ID NOs: 453-902, 1485-2066, 2108-2147, 2188-2227, 2253-2277, 2302-2325 or 2340-2353 across the entire length of the sense strand. In some embodiments, the sense strand comprises a nucleotide sequence having less than or equal to 1 nucleobase differences to the nucleotide sequence of any one of SEQ ID NOs: 453-902, 1485-2066, 2108-2147, 2188-2227, 2253-2277, 2302-2325 or 2340-2353 across the entire length of the sense strand. In some embodiments, the sense strand comprises a nucleotide sequence having 0 nucleobase differences to the nucleotide sequence of any one of SEQ ID NOs: 453-902, 1485-2066, 2108-2147, 2188-2227, 2253-2277, 2302-2325 or 2340-2353 across the entire length of the sense strand.

In some embodiments, the sense strand comprises a nucleotide sequence of any of the sense strands listed in Table 1 or Table 1A or Table 2. In some embodiments, the sense strand comprises a nucleotide sequence of any of the sense strands listed in Table 1. In some embodiments, the sense strand comprises a nucleotide sequence of any of the sense strands listed in Table 1A. In some embodiments, the sense strand comprises a nucleotide sequence of any of the sense strands listed in Table 2.

In some embodiments, the sense strand may comprise an overhang sequence. In some embodiments, the overhang sequence comprises at least about 1, 2, 3, 4, or 5 or more nucleotides. In some embodiments, the overhang sequence comprises at least about 1 nucleotide. In some embodiments, the overhang sequence comprises at least about 2 nucleotides. In some embodiments, the overhang sequence comprises at least about 3 nucleotides. In some embodiments, the overhang sequence comprises at least about 4 nucleotides. In some embodiments, the overhang sequence comprises at least about 5 nucleotides. In some embodiments, the overhang sequence comprises a UU sequence.

In some embodiments, the sense strand may comprise at least 1, 2, 3, or 4 phosphorothioate internucleoside linkages. In some embodiments, at least one phosphorothioate internucleoside linkage is between the nucleotides at positions 1 and 2 from the 5′ end of the sense strand. In some embodiments, at least one phosphorothioate internucleoside linkage is between the nucleotides at positions 2 and 3 from the 5′ end of the sense strand. In some embodiments, at least one phosphorothioate internucleoside linkage is between the nucleotides at positions 1 and 2 from the 3′ end of the sense strand. In some embodiments, at least one phosphorothioate internucleoside linkage is between the nucleotides at positions 2 and 3 from the 3′ end of the sense strand.

In some embodiments, the sense strand may comprise a nucleotide sequence comprising 2′-fluoro nucleotides at positions 5 and 7-9 from the 5′ end of the nucleotide sequence. In some embodiments, the sense strand may comprise a nucleotide sequence comprising 2′-fluoro nucleotides at positions 7 and 9-11 from the 5′ end of the nucleotide sequence. In some embodiments, the sense strand may comprise a nucleotide comprising 2′-fluoro nucleotides at positions 5, 9-11, 14, and 19 from the 5′ end of the nucleotide sequence.

In some embodiments, the sense strand may comprise a nucleotide sequence consisting of 19 to 23, or 19 to 21, nucleotides, wherein 2′-fluoro nucleotides are at positions 5 and 7-9 from the 5′ end of the nucleotide sequence. In some embodiments, the sense strand may comprise a nucleotide sequence consisting of 19 to 23, or 19 to 21, nucleotides, wherein 2′-fluoro nucleotides are at positions 7 and 9-11 from the 5′ end of the nucleotide sequence. In some embodiments, the sense strand may comprise a nucleotide sequence consisting of 19 to 23, or 19 to 21, nucleotides, wherein 2′-fluoro nucleotides are at positions 5, 9-11, 14, and 19 from the 5′ end of the nucleotide sequence.

### Antisense Strand

Any of the siRNA molecules described herein may comprise an antisense strand. In some embodiments, the antisense strand comprises between about 15 to about 50 nucleotides. In some embodiments, the antisense strand comprises between about 15 to about 45 nucleotides. In some embodiments, the antisense strand comprises between about 15 to about 40 nucleotides. In some embodiments, the antisense strand comprises between about 15 to about 35 nucleotides. In some embodiments, the antisense strand comprises between about 15 to about 30 nucleotides. In some embodiments, the antisense strand comprises between about 15 to about 25 nucleotides. In some embodiments, the antisense strand comprises between about 17 to about 23 nucleotides. In some embodiments, the antisense strand comprises between about 17 to about 22 nucleotides. In some embodiments, the antisense strand comprises between about 17 to about 21 nucleotides. In some embodiments, the antisense strand comprises between about 18 to about 23 nucleotides. In some embodiments, the antisense strand comprises between about 18 to about 22 nucleotides. In some embodiments, the antisense strand comprises between about 18 to about 21 nucleotides. In some embodiments, the antisense strand comprises between about 19 to about 23 nucleotides. In some embodiments, the antisense strand comprises between about 19 to about 22 nucleotides. In some embodiments, the antisense strand comprises between about 19 to about 21 nucleotides.

In some embodiments, the antisense strand comprises at least about 15, 16, 17, 18, 19, 20, 21, 22, 23, 24, 25, 26, 27, 28, 29, or 30 or more nucleotides. In some embodiments, the antisense strand comprises at least about 15 nucleotides. In some embodiments, the antisense strand comprises at least about 16 nucleotides. In some embodiments, the antisense strand comprises at least about 17 nucleotides. In some embodiments, the antisense strand comprises at least about 18 nucleotides. In some embodiments, the antisense strand comprises at least about 19 nucleotides. In some embodiments, the antisense strand comprises at least about 20 nucleotides. In some embodiments, the antisense strand comprises at least about 21 nucleotides. In some embodiments, the antisense strand comprises at least about 22 nucleotides. In some embodiments, the antisense strand comprises at least about 23 nucleotides.

In some embodiments, the antisense strand comprises less than about 50, 45, 40, 35, 30, 29, 28, 27, 26, 25, 24, 23, 22, 21, 20, or 19 or fewer nucleotides. In some embodiments, the antisense strand comprises less than about 30 nucleotides. In some embodiments, the antisense strand comprises less than about 25 nucleotides. In some embodiments, the antisense strand comprises less than about 24 nucleotides. In some embodiments, the antisense strand comprises less than about 23 nucleotides. In some embodiments, the antisense strand comprises less than about 22 nucleotides. In some embodiments, the antisense strand comprises less than about 21 nucleotides. In some embodiments, the antisense strand comprises less than about 20 nucleotides. In some embodiments, the antisense strand comprises less than about 19 nucleotides.

In some embodiments, the antisense strand comprises a sequence that is at least about 60%, 65%, 70%, 75%, 80%, 85%, 90%, 95%, or 100% complementary to a fragment of the PNPLA3 gene across the entire length of the antisense strand. In some embodiments, the antisense strand comprises a sequence that is at least about 70% complementary to a fragment of the PNPLA3 gene across the entire length of the antisense strand. In some embodiments, the antisense strand comprises a sequence that is at least about 75% complementary to a fragment of the PNPLA3 gene across the entire length of the antisense strand. In some embodiments, the antisense strand comprises a sequence that is at least about 80% complementary to a fragment of the PNPLA3 gene across the entire length of the antisense strand. In some embodiments, the antisense strand comprises a sequence that is at least about 85% complementary to a fragment of the PNPLA3 gene across the entire length of the antisense strand. In some embodiments, the antisense strand comprises a sequence that is at least about 90% complementary to a fragment of the PNPLA3 gene across the entire length of the antisense strand. In some embodiments, the antisense strand comprises a sequence that is at least about 95% complementary to a fragment of the PNPLA3 gene across the entire length of the antisense strand. In some embodiments, the antisense strand comprises a sequence that is about 100% complementary to a fragment of the PNPLA3 gene across the entire length of the antisense strand. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the antisense strand comprises a sequence having between about 15 to about 50 consecutive nucleotides complementary to a fragment of the PNPLA3 gene. In some embodiments, the antisense strand comprises a sequence having between about 15 to about 45 consecutive nucleotides complementary to a fragment of the PNPLA3 gene. In some embodiments, the antisense strand comprises a sequence having between about 15 to about 40 consecutive nucleotides complementary to a fragment of the PNPLA3 gene. In some embodiments, the antisense strand comprises a sequence having between about 15 to about 35 consecutive nucleotides complementary to a fragment of the PNPLA3 gene. In some embodiments, the antisense strand comprises a sequence having between about 15 to about 30 consecutive nucleotides complementary to a fragment of the PNPLA3 gene. In some embodiments, the antisense strand comprises a sequence having between about 15 to about 25 consecutive nucleotides complementary to a fragment of the PNPLA3 gene. In some embodiments, the antisense strand comprises between about 17 to about 23 consecutive nucleotides complementary to a fragment of the PNPLA3 gene. In some embodiments, the antisense strand comprises between about 17 to about 22 consecutive nucleotides complementary to a fragment of the PNPLA3 gene. In some embodiments, the antisense strand comprises between about 17 to about 21 consecutive nucleotides complementary to a fragment of the PNPLA3 gene. In some embodiments, the antisense strand comprises between about 18 to about 23 consecutive nucleotides complementary to a fragment of the PNPLA3 gene. In some embodiments, the antisense strand comprises between about 18 to about 22 consecutive nucleotides complementary to a fragment of the PNPLA3 gene. In some embodiments, the antisense strand comprises between about 18 to about 21 consecutive nucleotides complementary to a fragment of the PNPLA3 gene. In some embodiments, the antisense strand comprises between about 19 to about 23 consecutive nucleotides complementary to a fragment of the PNPLA3 gene. In some embodiments, the antisense strand comprises between about 19 to about 22 consecutive nucleotides of a fragment of the PNPLA3 gene. In some embodiments, the antisense strand comprises between about 19 to about 21 consecutive nucleotides complementary to a fragment of the PNPLA3 gene. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the antisense strand comprises a sequence having at least about 15, 16, 17, 18, 19, 20, 21, 22, 23, 24, 25, 26, 27, 28, 29, or 30 or more consecutive nucleotides complementary to a fragment of the PNPLA3 gene. In some embodiments, the antisense strand comprises a sequence having at least about 15 consecutive nucleotides complementary to a fragment of the PNPLA3 gene. In some embodiments, the antisense strand comprises a sequence having at least about 16 consecutive nucleotides complementary to a fragment of the PNPLA3 gene. In some embodiments, the antisense strand comprises a sequence having at least about 17 consecutive nucleotides complementary to a fragment of the PNPLA3 gene. In some embodiments, the antisense strand comprises a sequence having at least about 18 consecutive nucleotides complementary to a fragment of the PNPLA3 gene. In some embodiments, the antisense strand comprises a sequence having at least about 19 consecutive nucleotides complementary to a fragment of the PNPLA3 gene. In some embodiments, the antisense strand comprises a sequence having at least about 20 consecutive nucleotides complementary to a fragment of the PNPLA3 gene. In some embodiments, the antisense strand comprises a sequence having at least about 21 consecutive nucleotides complementary to a fragment of the PNPLA3 gene. In some embodiments, the antisense strand comprises a sequence having at least about 22 consecutive nucleotides complementary to a fragment of the PNPLA3 gene. In some embodiments, the antisense strand comprises a sequence having at least about 23 consecutive nucleotides complementary to a fragment of the PNPLA3 gene. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the antisense strand comprises a sequence having less than about 50, 45, 40, 35, 30, 29, 28, 27, 26, 25, 24, 23, 22, 21, 20, or 19 or fewer consecutive nucleotides complementary to a fragment of the PNPLA3 gene. In some embodiments, the antisense strand comprises a sequence having less than about 35 consecutive nucleotides complementary to a fragment of the PNPLA3 gene. In some embodiments, the antisense strand comprises a sequence having less than about 30 consecutive nucleotides complementary to a fragment of the PNPLA3 gene. In some embodiments, the antisense strand comprises a sequence having less than about 25 consecutive nucleotides complementary to a fragment of the PNPLA3 gene. In some embodiments, the antisense strand comprises a sequence having less than about 24 consecutive nucleotides complementary to a fragment of the PNPLA3 gene. In some embodiments, the antisense strand comprises a sequence having less than about 23 consecutive nucleotides complementary to a fragment of the PNPLA3 gene. In some embodiments, the antisense strand comprises a sequence having less than about 22 consecutive nucleotides complementary to a fragment of the PNPLA3 gene. In some embodiments, the antisense strand comprises a sequence having less than about 21 consecutive nucleotides complementary to a fragment of the PNPLA3 gene. In some embodiments, the antisense strand comprises a sequence having less than about 20 consecutive nucleotides complementary to a fragment of the PNPLA3 gene. In some embodiments, the antisense strand comprises a sequence having less than about 19 consecutive nucleotides complementary to a fragment of the PNPLA3 gene. In some embodiments, the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the antisense strand comprises a sequence having less than or equal to 5, 4, 3, 2, or 1 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the antisense strand comprises a sequence having less than or equal to 5 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the antisense strand comprises a sequence having less than or equal to 4 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the antisense strand comprises a sequence having less than or equal to 3 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the antisense strand comprises a sequence having less than or equal to 2 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the antisense strand comprises a sequence having less than or equal to 1 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the antisense strand comprises a sequence having 0 mismatches to a fragment of the PNPLA3 gene across the entire length of the antisense strand, wherein the fragment of the PNPLA3 gene consists of about 15, 16, 17, 18, 19, 20, 21, 22, 23, 24, 25, 26, 27, 28, 29, or 30 consecutive nucleotides of the PNPLA3 gene. In some embodiments, the fragment of the PNPLA3 gene consists of about 15 consecutive nucleotides of the PNPLA3 gene. In some embodiments, the fragment of the PNPLA3 gene consists of about 16 consecutive nucleotides of the PNPLA3 gene. In some embodiments, the fragment of the PNPLA3 gene consists of about 17 consecutive nucleotides of the PNPLA3 gene. In some embodiments, the fragment of the PNPLA3 gene consists of about 18 consecutive nucleotides of the PNPLA3 gene. In some embodiments, the fragment of the PNPLA3 gene consists of about 19 consecutive nucleotides of the PNPLA3 gene. In some embodiments, the fragment of the PNPLA3 gene consists of about 20 consecutive nucleotides of the PNPLA3 gene. In some embodiments, the fragment of the PNPLA3 gene consists of about 21 consecutive nucleotides of the PNPLA3 gene. In some embodiments, the fragment of the PNPLA3 gene consists of about 22 consecutive nucleotides of the PNPLA3 gene. In some embodiments, the fragment of the PNPLA3 gene consists of about 23 consecutive nucleotides of the PNPLA3 gene.

In some embodiments, the antisense strand comprises a nucleotide sequence of any one of SEQ ID NOs: 453-902, 1485-2066, 2108-2147, 2188-2227, 2253-2277, 2302-2325 or 2340-2353. In some embodiments, the antisense strand comprises a nucleotide sequence that is at least about 60%, 65%, 70%, 75%, 80%, 85%, 90%, 95%, or 100% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is at least about 70% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is at least about 75% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is at least about 80% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is at least about 85% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is at least about 90% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is at least about 95% identical to the nucleotide sequence of any one of SEQ ID NOs: 453-902, 1485-2066, 2108-2147, 2188-2227, 2253-2277, 2302-2325 or 2340-2353 across the entire length of antisense strand. In some embodiments, the antisense strand comprises a nucleotide sequence that is about 100% identical to the nucleotide sequence of any one of SEQ ID NOs: 453-902, 1485-2066, 2108-2147, 2188-2227, 2253-2277, 2302-2325 or 2340-2353 across the entire length of antisense strand.

In some embodiments, the antisense strand comprises at least about 15, 16, 17, 18, 19, 20, 21, 22, or 23 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 17 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 18 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 19 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 20 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 21 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 22 consecutive nucleotides of the nucleotide sequence of any one of SEQ ID NOs: 453-902, 1485-2066, 2108-2147, 2188-2227, 2253-2277, 2302-2325 or 2340-2353. In some embodiments, the antisense strand comprises at least about 23 consecutive nucleotides of the nucleotide sequence of any one of SEQ ID NOs: 453-902, 1485-2066, 2108-2147, 2188-2227, 2253-2277, 2302-2325 or 2340-2353.

In some embodiments, the antisense strand comprises a nucleotide sequence having less than or equal to 5, 4, 3, 2, or 1 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand. In some embodiments, the antisense strand comprises a nucleotide sequence having less than or equal to 5 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand. In some embodiments, the antisense strand comprises a nucleotide sequence having less than or equal to 4 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand. In some embodiments, the antisense strand comprises a nucleotide sequence having less than or equal to 3 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand. In some embodiments, the antisense strand comprises a nucleotide sequence having less than or equal to 2 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand. In some embodiments, the antisense strand comprises a nucleotide sequence having less than or equal to 1 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand. In some embodiments, the antisense strand comprises a nucleotide sequence having 0 mismatches to the nucleotide sequence of any one of SEQ ID NOs: 3-452, 903-1484, 2068-2107, 2148-2187, 2228-2253, 2278-2301, 2326-2339 or 2354-2358 across the entire length of the antisense strand.

In some embodiments, the antisense strand comprises a nucleotide sequence of any of the antisense strands listed in Table 1 or Table 1A or Table 2. In some embodiments, the antisense strand comprises a nucleotide sequence of any of the antisense strands listed in Table 1. In some embodiments, the antisense strand comprises a nucleotide sequence of any of the antisense strands listed in Table 1A. In some embodiments, the antisense strand comprises a nucleotide sequence of any of the antisense strands listed in Table 2.

In some embodiments, the antisense strand may comprise an overhang sequence. In some embodiments, the overhang sequence comprises at least about 1, 2, 3, 4, or 5 or more nucleotides. In some embodiments, the overhang sequence comprises at least about 1 nucleotide. In some embodiments, the overhang sequence comprises at least about 2 nucleotides. In some embodiments, the overhang sequence comprises at least about 3 nucleotides. In some embodiments, the overhang sequence comprises at least about 4 nucleotides. In some embodiments, the overhang sequence comprises at least about 5 nucleotides. In some embodiments, the overhang sequence comprises a UU sequence.

In some embodiments, the antisense strand may comprise at least 1, 2, 3, or 4 phosphorothioate internucleoside linkages. In some embodiments, at least one phosphorothioate internucleoside linkage is between the nucleotides at positions 1 and 2 from the 5′ end of the antisense strand. In some embodiments, at least one phosphorothioate internucleoside linkage is between the nucleotides at positions 2 and 3 from the 5′ end of the antisense strand. In some embodiments, at least one phosphorothioate internucleoside linkage is between the nucleotides at positions 1 and 2 from the 3′ end of the antisense strand. In some embodiments, at least one phosphorothioate internucleoside linkage is between the nucleotides at positions 2 and 3 from the 3′ end of the antisense strand.

In some embodiments, the antisense strand may comprise a nucleotide sequence comprising 2′-fluoro nucleotides at positions 2, 6, 14, and 16 from the 5′ end of the nucleotide sequence. In some embodiments, the antisense strand may comprise a nucleotide sequence comprising 2′-fluoro nucleotides at positions 2 and 14 from the 5′ end of the nucleotide sequence. In some embodiments, the antisense strand may comprise a nucleotide sequence comprising 2′-fluoro nucleotides at positions 2, 5, 8, 14, and 17 from the 5′ end of the nucleotide sequence.

In some embodiments, the antisense strand may comprise a nucleotide sequence consisting of 17 to 23, or 19 to 23, nucleotides, wherein 2′-fluoro nucleotides are at positions 2, 6, 14, and 16 from the 5′ end of the nucleotide sequence. In some embodiments, the antisense strand may comprise a nucleotide sequence consisting of 17 to 23, or 19 to 23, nucleotides, wherein 2′-fluoro nucleotides are at positions 2 and 14 from the 5′ end of the nucleotide sequence. In some embodiments, the antisense strand may comprise a nucleotide sequence consisting of 17 to 23, or 19 to 23, nucleotides, wherein 2′-fluoro nucleotides are at positions 2, 5, 8, 14, and 17 from the 5′ end of the nucleotide sequence.

Modified siRNAs

In some embodiments, the siRNA molecules disclosed herein may be chemically modified. In some embodiments, the siRNA molecules may be modified, for example, to enhance stability and/or bioavailability and/or provide otherwise beneficial characteristics in vitro, in vivo, and/or ex vivo. For example, siRNA molecules may be modified such that the two strands (sense and antisense) maintain the ability to hybridize to each other and/or the siRNA molecules maintain the ability to hybridize to a target sequence. Examples of siRNA modifications include modifications to the ribose sugar, nucleobase, and/or phosphodiester backbone, including but not limited to those described herein. Non-limiting examples of siRNA modifications are described, e.g., in WO 2020/243490; WO 2020/097342; WO 2021/119325; PCT/US2021/019629; PCT/US2021/019628; PCT/US2021/021199; Sig. Transduct. Target Ther. 5 (101), 1-25, 2020; and J. Am. Chem. Soc. 136 (49), 16958-16961, 2014, the contents of each of which are hereby incorporated herein by reference in their entirety.

In some embodiments, the siRNA molecules disclosed herein comprise modified nucleotides having a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than H or OH. Such 2′ modifications include, but are not limited to, 2′-OH, 2′-S-alkyl, 2′-N-alkyl, 2′-O-alkyl, 2′-S-alkenyl, 2′-N-alkenyl, 2′-O-alkenyl, 2′-S-alkynyl, 2′-N-alkynyl, 2′-O-alkynyl, 2′-O-allyl, 2′-C-allyl, 2′-fluoro, 2′-O-methyl (OMe or OCH₃), 2′-O-methoxyethyl, 2′-ara-F, 2′-OCF₃, 2′-O(CH₂)₂SCH₃, 2′-O-aminoalkyl, 2′-amino (e.g. NH₂), 2′-O-ethylamine, and 2′-azido, wherein the alkyl, alkenyl and alkynyl can be substituted or unsubstituted. Modifications at the 5′ position of the pentose ring include, but are not limited to, 5′-methyl (R or S), 5′-vinyl, and 5′-methoxy. Sugar modifications may also include, for example, LNA, UNA, GNA, and DNA. In some embodiments, the siRNA molecules of the disclosure comprise one or more 2′-O-methyl nucleotides, 2′-fluoro nucleotides, or combinations thereof.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of any sense or antisense nucleotide sequences described herein are 2′-O-methyl nucleotides. In some embodiments, between about 2 to 20 modified nucleotides of any sense or antisense nucleotide sequences described herein are 2′-O-methyl nucleotides. In some embodiments, between about 5 to 25 modified nucleotides of any sense or antisense nucleotide sequences described herein are 2′-O-methyl nucleotides. In some embodiments, between about 10 to 25 modified nucleotides of any sense or antisense nucleotide sequences described herein are 2′-O-methyl nucleotides. In some embodiments, between about 12 to 25 modified nucleotides of any sense or antisense nucleotide sequences described herein are 2′-O-methyl nucleotides. In some embodiments, at least about 10, 11, 12, 13, 14, 15, 16, 17, 18, 19, 20, 21, or 22 modified nucleotides of any sense or antisense nucleotide sequences described herein are 2′-O-methyl nucleotides. In some embodiments, at least about 12 modified nucleotides of any sense or antisense nucleotide sequences described herein are 2′-O-methyl nucleotides. In some embodiments, at least about 13 modified nucleotides of any sense or antisense nucleotide sequences described herein are 2′-O-methyl nucleotides. In some embodiments, at least about 14 modified nucleotides of any sense or antisense nucleotide sequences described herein are 2′-O-methyl nucleotides. In some embodiments, at least about 15 modified nucleotides of any sense or antisense nucleotide sequences described herein are 2′-O-methyl nucleotides. In some embodiments, at least about 16 modified nucleotides of any sense or antisense nucleotide sequences described herein are 2′-O-methyl nucleotides. In some embodiments, at least about 17 modified nucleotides of any sense or antisense nucleotide sequences described herein are 2′-O-methyl nucleotides. In some embodiments, at least about 18 modified nucleotides of any sense or antisense nucleotide sequences described herein are 2′-O-methyl nucleotides. In some embodiments, at least about 19 modified nucleotides of any sense or antisense nucleotide sequences described herein are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of any sense or antisense nucleotide sequences described herein are 2′-O-methyl nucleotides. In some embodiments, less than or equal to 21 modified nucleotides of any sense or antisense nucleotide sequences described herein are 2′-O-methyl nucleotides. In some embodiments, less than or equal to 20 modified nucleotides of any sense or antisense nucleotide sequences described herein are 2′-O-methyl nucleotides. In some embodiments, less than or equal to 19 modified nucleotides of any sense or antisense nucleotide sequences described herein are 2′-O-methyl nucleotides. In some embodiments, less than or equal to 18 modified nucleotides of any sense or antisense nucleotide sequences described herein are 2′-O-methyl nucleotides. In some embodiments, less than or equal to 17 modified nucleotides of any sense or antisense nucleotide sequences described herein are 2′-O-methyl nucleotides. In some embodiments, less than or equal to 16 modified nucleotides of any sense or antisense nucleotide sequences described herein are 2′-O-methyl nucleotides. In some embodiments, less than or equal to 15 modified nucleotides of any sense or antisense nucleotide sequences described herein are 2′-O-methyl nucleotides. In some embodiments, less than or equal to 14 modified nucleotides of any sense or antisense nucleotide sequences described herein are 2′-O-methyl nucleotides. In some embodiments, less than or equal to 13 modified nucleotides of any sense or antisense nucleotide sequences described herein are 2′-O-methyl nucleotides. In some embodiments, at least one modified nucleotide of any sense or antisense nucleotide sequences described herein is a 2′-O-methyl pyrimidine. In some embodiments, at least 5, 6, 7, 8, 9, or 10 modified nucleotides of any sense or antisense nucleotide sequences described herein are 2′-O-methyl pyrimidines. In some embodiments, at least one modified nucleotide of any sense or antisense nucleotide sequences described herein is a 2′-O-methyl purine. In some embodiments, at least 5, 6, 7, 8, 9, or 10 modified nucleotides of any sense or antisense nucleotide sequences described herein are 2′-O-methyl purines. In some embodiments, the 2′-O-methyl nucleotide is a 2′-O-methyl nucleotide mimic.

In some embodiments, the nucleotide at position 3, 5, 7, 8, 9, 10, 11, 12, 14, 17, and/or 19 from the 5′ end of any sense or antisense nucleotide sequences described herein is a 2′-fluoro nucleotide. In some embodiments, at least two nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, at least three nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, at least four nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, at least five nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, the nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, the nucleotide at position 3 from the 5′ end of any sense or antisense nucleotide sequences described herein is a 2′-fluoro nucleotide. In some embodiments, the nucleotide at position 7 from the 5′ end of any sense or antisense nucleotide sequences described herein is a 2′-fluoro nucleotide. In some embodiments, the nucleotide at position 8 from the 5′ end of any sense or antisense nucleotide sequences described herein is a 2′-fluoro nucleotide. In some embodiments, the nucleotide at position 9 from the 5′ end of any sense or antisense nucleotide sequences described herein is a 2′-fluoro nucleotide. In some embodiments, the nucleotide at position 12 from the 5′ end of any sense or antisense nucleotide sequences described herein is a 2′-fluoro nucleotide. In some embodiments, the nucleotide at position 17 from the 5′ end of any sense or antisense nucleotide sequences described herein is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 1, 3, 5, 7, 8, 9, 10, 11, 12, 14, 17, and/or 19 from the 5′ end of any sense or antisense nucleotide sequences described herein is a 2′-fluoro nucleotide. In some embodiments, the nucleotide at positions 1, 3, 5, 7, 8, 9, 10, 11, 12, 14, 17, and/or 19 from the 5′ end of any sense or antisense nucleotide sequences described herein is a 2′-fluoro nucleotide. In some embodiments, at least two nucleotides at positions 1, 3, 5, 7, 8, 9, 10, 11, 12, 14, 17, and/or 19 from the 5′ end of any sense or antisense nucleotide sequences described herein are 2′-fluoro nucleotides. In some embodiments, at least three nucleotides at positions 1, 3, 5, 7, 8, 9, 10, 11, 12, 14, 17, and/or 19 from the 5′ end of any sense or antisense nucleotide sequences described herein are 2′-fluoro nucleotides. In some embodiments, the nucleotides at positions 1, 3, 5, 7, 8, 9, 10, 11, 12, 14, 17, and/or 19 from the 5′ end of any sense or antisense nucleotide sequences described herein are 2′-fluoro nucleotides. In some embodiments, the 2′-fluoro nucleotide is a 2′-fluoro nucleotide mimic.

In some embodiments, at least 1, 2, 3, 4, 5, 6, or 7 nucleotides at position 2, 4, 6, 8, 10, 12, 14, 16, and/or 18 from the 5′ end of any sense or antisense nucleotide sequences described herein is a 2′-fluoro nucleotide. In some embodiments, the nucleotide at positions, 4, 6, 8, 10, 12, 14, 16, and/or 18 from the 5′ end of any sense or antisense nucleotide sequences described herein is a 2′-fluoro nucleotide. In some embodiments, at least two nucleotides at positions 2, 4, 6, 8, 10, 12, 14, 16, and/or 18 from the 5′ end of any sense or antisense nucleotide sequences described herein are 2′-fluoro nucleotides. In some embodiments, at least three nucleotides at positions 2, 4, 6, 8, 10, 12, 14, 16, and/or 18 from the 5′ end of any sense or antisense nucleotide sequences described herein are 2′-fluoro nucleotides. In some embodiments, the nucleotides at positions 2, 4, 6, 8, 10, 12, 14, 16, and/or 18 from the 5′ end of any sense or antisense nucleotide sequences described herein are 2′-fluoro nucleotides. In some embodiments, the 2′-fluoro nucleotide is a 2′-fluoro nucleotide mimic.

In some embodiments, the nucleotide at position 1 from the 5′ end of any sense nucleotide sequences described herein is a 2′-fluoro nucleotide. In some embodiments, the nucleotide at position 3 from the 5′ end of any sense nucleotide sequences described herein is a 2′-fluoro nucleotide. In some embodiments, the nucleotide at position 5 from the 5′ end of any sense nucleotide sequences described herein is a 2′-fluoro nucleotide. In some embodiments, the nucleotide at position 7 from the 5′ end of any sense nucleotide sequences described herein is a 2′-fluoro nucleotide. In some embodiments, the nucleotide at position 8 from the 5′ end of any sense nucleotide sequences described herein is a 2′-fluoro nucleotide. In some embodiments, the nucleotide at position 9 from the 5′ end of any sense nucleotide sequences described herein is a 2′-fluoro nucleotide. In some embodiments, the nucleotide at position 10 from the 5′ end of any sense nucleotide sequences described herein is a 2′-fluoro nucleotide. In some embodiments, the nucleotide at position 11 from the 5′ end of any sense nucleotide sequences described herein is a 2′-fluoro nucleotide. In some embodiments, the nucleotide at position 12 from the 5′ end of any sense nucleotide sequences described herein is a 2′-fluoro nucleotide. In some embodiments, the nucleotide at position 14 from the 5′ end of any sense nucleotide sequences described herein is a 2′-fluoro nucleotide. In some embodiments, the nucleotide at position 17 from the 5′ end of any sense nucleotide sequences described herein is a 2′-fluoro nucleotide. In some embodiments, the nucleotide at position 19 from the 5′ end of any sense nucleotide sequences described herein is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 1, 3, 5, 7, 8, 9, 10, 11, 12, 14, 17, and/or 19 from the 5′ end of any sense nucleotide sequences described herein is a 2′-fluoro nucleotide. In some embodiments, the nucleotide at position 5, 7, 8, 9, 10, 11, 14, and/or 19 from the 5′ end of any sense nucleotide sequences described herein is a 2′-fluoro nucleotide. In some embodiments, the nucleotide at position 5, 7, 8, and/or 9 from the 5′ end of any sense nucleotide sequences described herein is a 2′-fluoro nucleotide. In some embodiments, the nucleotide at position 7, 9, 10, and/or 11 from the 5′ end of any sense or antisense nucleotide sequences described herein is a 2′-fluoro nucleotide. In some embodiments, the nucleotide at position 5, 9, 10, 11, 14, and/or 19 from the 5′ end of any sense nucleotide sequences described herein is a 2′-fluoro nucleotide. In some embodiments, the 2′-fluoro nucleotide is a 2′-fluoro nucleotide mimic.

In some embodiments, the nucleotide at position 2 from the 5′ end of any antisense nucleotide sequences described herein is a 2′-fluoro nucleotide. In some embodiments, the nucleotide at position 4 from the 5′ end of any antisense nucleotide sequences described herein is a 2′-fluoro nucleotide. In some embodiments, the nucleotide at position 6 from the 5′ end of any antisense nucleotide sequences described herein is a 2′-fluoro nucleotide. In some embodiments, the nucleotide at position 8 from the 5′ end of any antisense nucleotide sequences described herein is a 2′-fluoro nucleotide. In some embodiments, the nucleotide at position 10 from the 5′ end of any antisense nucleotide sequences described herein is a 2′-fluoro nucleotide. In some embodiments, the nucleotide at position 12 from the 5′ end of any antisense nucleotide sequences described herein is a 2′-fluoro nucleotide. In some embodiments, the nucleotide at position 14 from the 5′ end of any antisense nucleotide sequences described herein is a 2′-fluoro nucleotide. In some embodiments, the nucleotide at position 16 from the 5′ end of any antisense nucleotide sequences described herein is a 2′-fluoro nucleotide. In some embodiments, the nucleotide at position 18 from the 5′ end of any antisense nucleotide sequences described herein is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 2, 4, 5, 6, 8, 10, 12, 14, 16, 17 and/or 18 from the 5′ end of any antisense nucleotide sequences described herein is a 2′-fluoro nucleotide. In some embodiments, the nucleotide at position 2, 5, 6, 8, 14, 16, and/or 17 from the 5′ end of any antisense nucleotide sequences described herein is a 2′-fluoro nucleotide. In some embodiments, the nucleotide at position 2, 6, 14, and/or 16 from the 5′ end of any antisense nucleotide sequences described herein is a 2′-fluoro nucleotide. In some embodiments, the nucleotide at position 2, and/or 14 from the 5′ end of any antisense nucleotide sequences described herein is a 2′-fluoro nucleotide. In some embodiments, the nucleotide at position 2, 5, 8, 14, and/or 17 from the 5′ end of any antisense nucleotide sequences described herein is a 2′-fluoro nucleotide. In some embodiments, the 2′-fluoro nucleotide is a 2′-fluoro nucleotide mimic.

In some embodiments, the 2′-fluoro or 2′-O-methyl nucleotide mimic is a nucleotide mimic of Formula (V):

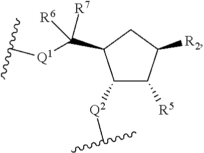

wherein Rₓ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16)-Formula (20):

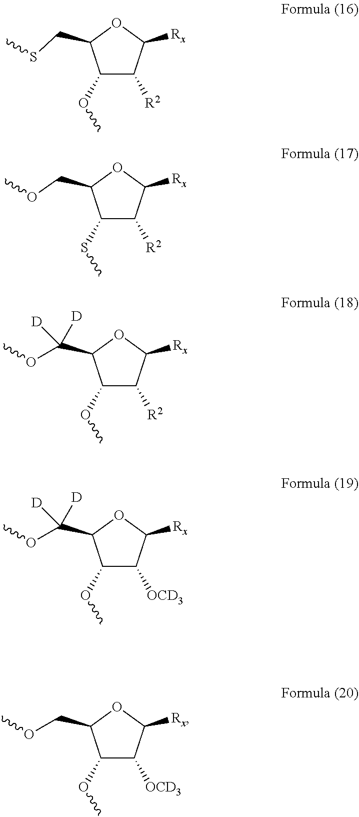

wherein Rₓ is independently a nucleobase and R² is F or —OCH₃. In some embodiments, the nucleobase is selected from thymine, cytosine, guanine, adenine, uracil, and an analogue or derivative thereof.

In some embodiments, the sense strand or the antisense strand may comprise at least 1, at least 2, at least 3, at least 4, or at least 5 or more modified nucleotide(s) having the following chemical structure:

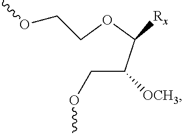

wherein Rₓ is a nucleobase, aryl, heteroaryl, or H. In some embodiments, the nucleobase is selected from thymine, cytosine, guanine, adenine, uracil, and an analogue or derivative thereof.

In some embodiments, the sense strand or the antisense strand may comprise at least 1, at least 2, at least 3, at least 4, or at least 5 or more modified nucleotide(s) having the following chemical structure:

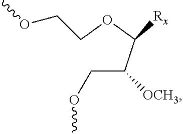

wherein Rₓ is a nucleobase. In some embodiments, the nucleobase is selected from thymine, cytosine, guanine, adenine, uracil, and an analogue or derivative thereof.

In some embodiments, the sense strand or the antisense strand may comprise at least 1, at least 2, at least 3, at least 4, or at least 5 or more modified nucleotide(s) having the following chemical structure:

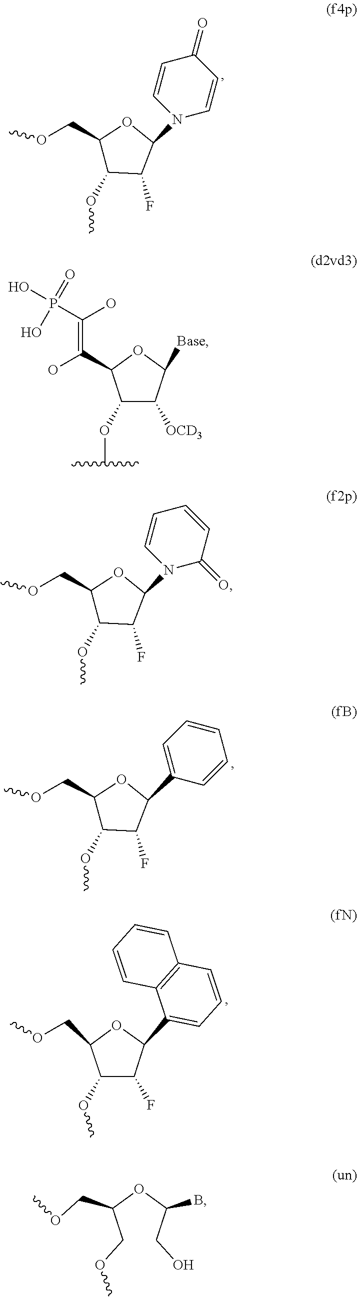

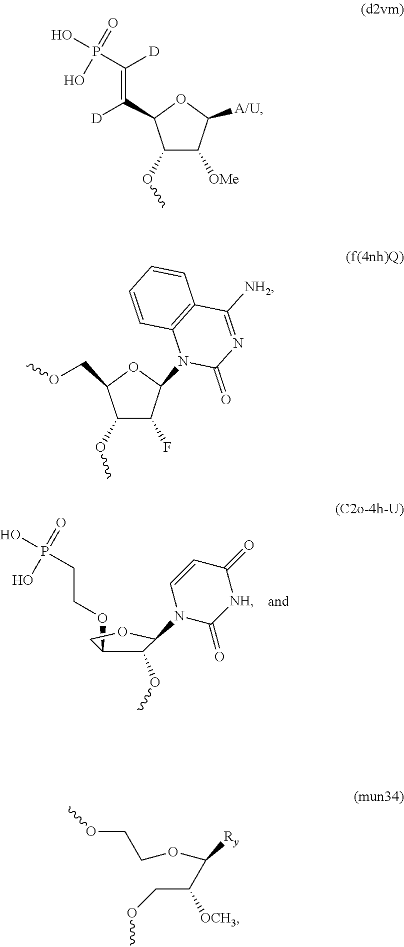

wherein B and Ry is a nucleobase. In some embodiments, the nucleobase is selected from thymine, cytosine, guanine, adenine, uracil, and an analogue or derivative thereof.

In some embodiments, any sense or antisense nucleotide sequence described herein comprises, consists of, or consists essentially of ribonucleic acids (RNAs). In some embodiments, any sense or antisense nucleotide sequence described herein comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of any sense or antisense nucleotide sequence described herein are independently selected from 2′-O-methyl RNA and 2′-fluoro RNA.

In some embodiments, the siRNA molecules disclosed herein include end modifications at the 5′ end and/or the 3′ end of the sense strand and/or the antisense strand. In some embodiments, the siRNA molecules disclosed herein comprise a phosphate moiety at the 5′ end of the sense strand and/or antisense strand. In some embodiments, the 5′ end of the sense strand and/or antisense strand comprises a phosphate mimic or analogue (e.g., “5′ terminal phosphate mimic”). In some embodiments, the 5′ end of the sense strand and/or antisense strand comprises a vinyl phosphonate or a variation thereof (e.g., “5′ terminal vinyl phosphonate”).

In some embodiments, the siRNA molecules comprise at least one backbone modification, such as a modified internucleoside linkage. In some embodiments, the siRNA molecules described herein comprise at least one phosphorothioate internucleoside linkage. In particular embodiments, the phosphorothioate internucleoside linkages may be positioned at the 3′ or 5′ ends of the sense and/or antisense strands.

In some embodiments, siRNA molecules include an overhang of at least one unpaired nucleotide. In some embodiments in which the siRNA molecule comprises a nucleotide overhang, two or more of the unpaired nucleotides in the overhang can be connected by a phosphorothioate internucleoside linkage. In certain embodiments, all the unpaired nucleotides in a nucleotide overhang at the 3′ end of the antisense strand and/or the sense strand are connected by phosphorothioate internucleoside linkages. In some embodiments, all the unpaired nucleotides in a nucleotide overhang at the 5′ end of the antisense strand and/or the sense strand are connected by phosphorothioate internucleoside linkages. In some embodiments, all of the unpaired nucleotides in any nucleotide overhang are connected by phosphorothioate internucleoside linkages.

In some embodiments, the sense or the antisense strand may further comprise at least 1, 2, 3, 4, 5, 6, 7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of any sense or antisense nucleotide sequences described herein. In some embodiments, at least one phosphorothioate internucleoside linkage is between the nucleotides at positions 2 and 3 from the 5′ end of any sense or antisense nucleotide sequences described herein. In some embodiments, the sense strand comprises two phosphorothioate internucleoside linkages between the nucleotides at positions 1 to 3 from the 5′ end of any sense or antisense nucleotide sequences described herein.

In some embodiments, the modified nucleotides that can be incorporated into the siRNA molecules of the disclosure may have more than one chemical modification described herein. For instance, in some embodiments, the modified nucleotide may have a modification to the ribose sugar as well as a modification to the phosphodiester backbone. By way of example, a modified nucleotide may comprise a 2′ sugar modification (e.g., 2′-fluoro or 2′-O-methyl) and a modification to the 5′ phosphate that would create a modified internucleoside linkage when the modified nucleotide was incorporated into a polynucleotide. For instance, in some embodiments, the modified nucleotide may comprise a sugar modification, such as a 2′-fluoro modification or a 2′-O-methyl modification, for example, as well as a 5′ phosphorothioate group. In some embodiments, the sense and/or antisense strand of the siRNA molecules of the disclosure comprises a combination of 2′ modified nucleotides and phosphorothioate internucleoside linkages. In some embodiments, the sense and/or antisense strand of the siRNA molecules of the disclosure comprises a combination of 2′ sugar modifications, phosphorothioate internucleoside linkages, and 5′ terminal vinyl phosphonate.

In some embodiments, any of the siRNAs disclosed herein comprise 1, 2, 3, 4, 5, 6, 7, 8, 9, 10, 11, 12, 13, 14, 15, 16, 17, 18, 19, 20, 21, 22, 23, 24, 25, or more modified nucleotides. In some embodiments, any of the siRNAs disclosed herein comprise 1 or more modified nucleotides. In some embodiments, any of the siRNAs disclosed herein comprise 2 or more modified nucleotides. In some embodiments, any of the siRNAs disclosed herein comprise 5 or more modified nucleotides. In some embodiments, any of the siRNAs disclosed herein comprise 8 or more modified nucleotides. In some embodiments, any of the siRNAs disclosed herein comprise 10 or more modified nucleotides. In some embodiments, any of the siRNAs disclosed herein comprise 15 or more modified nucleotides. In some embodiments, any of the siRNAs disclosed herein comprise 20 or more modified nucleotides. In some embodiments, any of the siRNAs disclosed herein comprise 30 or more modified nucleotides. In some embodiments, any of the siRNAs disclosed herein comprise 35 or more modified nucleotides. In some embodiments, any of the siRNAs disclosed herein comprise 40 or more modified nucleotides. In some embodiments, any of the siRNAs disclosed herein comprise 45 or more modified nucleotides. In some embodiments, all of the nucleotides in the siRNA molecule are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, any of the sense strands disclosed herein comprise 1, 2, 3, 4, 5, 6, 7, 8, 9, 10, 11, 12, 13, 14, 15, 16, 17, 18, 19, 20, 21, 22, 23, 24, 25, or more modified nucleotides. In some embodiments, any of the sense strands disclosed herein comprise 1 or more modified nucleotides. In some embodiments, any of the sense strands disclosed herein comprise 2 or more modified nucleotides. In some embodiments, any of the sense strands disclosed herein comprise 5 or more modified nucleotides. In some embodiments, any of the sense strands disclosed herein comprise 8 or more modified nucleotides. In some embodiments, any of the sense strands disclosed herein comprise 10 or more modified nucleotides. In some embodiments, any of the sense strands disclosed herein comprise 15 or more modified nucleotides. In some embodiments, any of the sense strands disclosed herein comprise 17 or more modified nucleotides. In some embodiments, any of the sense strands disclosed herein comprise 18 or more modified nucleotides. In some embodiments, any of the sense strands disclosed herein comprise 19 or more modified nucleotides. In some embodiments, any of the sense strands disclosed herein comprise 20 or more modified nucleotides. In some embodiments, any of the sense strands disclosed herein comprise 21 or more modified nucleotides. In some embodiments, all of the nucleotides in the sense strand are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, any of the antisense strands disclosed herein comprise 1, 2, 3, 4, 5, 6, 7, 8, 9, 10, 11, 12, 13, 14, 15, 16, 17, 18, 19, 20, 21, 22, 23, 24, 25, or more modified nucleotides. In some embodiments, any of the antisense strands disclosed herein comprise 1 or more modified nucleotides. In some embodiments, any of the antisense strands disclosed herein comprise 2 or more modified nucleotides. In some embodiments, any of the antisense strands disclosed herein comprise 5 or more modified nucleotides. In some embodiments, any of the antisense strands disclosed herein comprise 8 or more modified nucleotides. In some embodiments, any of the antisense strands disclosed herein comprise 10 or more modified nucleotides. In some embodiments, any of the antisense strands disclosed herein comprise 15 or more modified nucleotides. In some embodiments, any of the antisense strands disclosed herein comprise 17 or more modified nucleotides. In some embodiments, any of the antisense strands disclosed herein comprise 18 or more modified nucleotides. In some embodiments, any of the antisense strands disclosed herein comprise 19 or more modified nucleotides. In some embodiments, any of the antisense strands disclosed herein comprise 20 or more modified nucleotides. In some embodiments, any of the antisense strands disclosed herein comprise 21 or more modified nucleotides. In some embodiments, any of the antisense strands disclosed herein comprise 22 or more modified nucleotides. In some embodiments, any of the antisense strands disclosed herein comprise 23 or more modified nucleotides. In some embodiments, all of the nucleotides in the antisense strand are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, at least about 10%, 20%, 30%, 40%, 45%, 50%, 55%, 60%, 65%, 70%, 75%, 80%, 90%, 95%, or 100% of the nucleotides in any of the sense strands disclosed herein are modified nucleotides. In some embodiments, at least about 10% of the nucleotides in any of the sense strands disclosed herein are modified nucleotides. In some embodiments, at least about 30% of the nucleotides in any of the sense strands disclosed herein are modified nucleotides. In some embodiments, at least about 50% of the nucleotides in any of the sense strands disclosed herein are modified nucleotides. In some embodiments, at least about 60% of the nucleotides in any of the sense strands disclosed herein are modified nucleotides. In some embodiments, at least about 70% of the nucleotides in any of the sense strands disclosed herein are modified nucleotides. In some embodiments, at least about 80% of the nucleotides in any of the sense strands disclosed herein are modified nucleotides. In some embodiments, at least about 90% of the nucleotides in any of the sense strands disclosed herein are modified nucleotides. In some embodiments, at least about 100% of the nucleotides in any of the sense strands disclosed herein are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, at least about 10%, 20%, 30%, 40%, 45%, 50%, 55%, 60%, 65%, 70%, 75%, 80%, 90%, 95%, or 100% of the nucleotides in any of the antisense strands disclosed herein are modified nucleotides. In some embodiments, at least about 10% of the nucleotides in any of the antisense strands disclosed herein are modified nucleotides. In some embodiments, at least about 30% of the nucleotides in any of the antisense strands disclosed herein are modified nucleotides. In some embodiments, at least about 50% of the nucleotides in any of the antisense strands disclosed herein are modified nucleotides. In some embodiments, at least about 60% of the nucleotides in any of the antisense strands disclosed herein are modified nucleotides. In some embodiments, at least about 70% of the nucleotides in any of the antisense strands disclosed herein are modified nucleotides. In some embodiments, at least about 80% of the nucleotides in any of the antisense strands disclosed herein are modified nucleotides. In some embodiments, at least about 90% of the nucleotides in any of the antisense strands disclosed herein are modified nucleotides. In some embodiments, at least about 100% of the nucleotides in any of the antisense strands disclosed herein are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, the siRNA molecule comprises a sense strand comprising a nucleotide sequence of any one of SEQ ID NOs: 903-1484, 2148-2187, 2278-2301, 2326-2339 or 2354-2358. In some embodiments, the siRNA molecule comprises an antisense strand comprising a nucleotide sequence of any one of SEQ ID NOs: 1485-2066, 2188-2227, 2302-2325 or 2340-2353. In some embodiments, the siRNA molecule comprises a sense strand comprising a nucleotide sequence of any one of SEQ ID NOs: 903-1484, 2148-2187, 2278-2301, 2326-2339 or 2354-2358 and an antisense strand comprising a nucleotide sequence of any one of SEQ ID NOs: 1485-2066, 2188-2227, 2302-2325 or 2340-2353.

siRNA Conjugates

In some embodiments, the siRNA molecules disclosed herein may comprise one or more conjugates or ligands. As used herein, a “conjugate” or “ligand” refers to any compound or molecule that is capable of interacting with another compound or molecule, directly or indirectly. In some embodiments, the ligand may modify one or more properties of the siRNA molecule to which it is attached, such as the pharmacodynamic, pharmacokinetic, binding, absorption, cellular distribution, cellular uptake, charge and/or clearance properties of the siRNA molecule. Non-limiting examples of such conjugates are described, e.g., in WO 2020/243490; WO 2020/097342; WO 2021/119325; PCT/US2021/019629; PCT/US2021/019628; PCT/US2021/021199; Sig. Transduct. Target Ther. 5 (101), 2020; ACS Chem. Biol. 10 (5), 1181-1187, 2015; J. Am. Chem. Soc. 136 (49), 16958-16961, 2014; Nucleic Acids Res. 42 (13), 8796-8807, 2014; Molec. Ther. 28 (8), 1759-1771, 2020; and Nucleic Acid Ther. 28 (3), 109-118, 2018, each of which is incorporated by reference herein.

In some embodiments, the ligand may be attached to the 5′ end and/or the 3′ end of the sense and/or antisense strand of the siRNA via covalent attachment such as to a nucleotide. In some embodiments, the ligand is covalently attached via a linker to the sense or antisense strand of the siRNA molecule. The ligand can be attached to nucleobases, sugar moieties, or internucleoside linkages of polynucleotides (e.g., sense strand or antisense strand) of the siRNA molecules of the disclosure.

In some embodiments, the type of conjugate or ligand used and the extent of conjugation of siRNA molecules of the disclosure can be evaluated, for example, for improved pharmacokinetic profiles, bioavailability, and/or stability of siRNA molecules while at the same time maintaining the ability of the siRNA to mediate RNAi activity. In some embodiments, a conjugate or ligand alters the distribution, targeting or lifetime of a siRNA molecule into which it is incorporated. In some embodiments, a conjugate or ligand provides an enhanced affinity for a selected target, e.g., molecule, cell or cell type, compartment (e.g., a cellular or organ compartment), tissue, organ or region of the body, as, e.g., compared to a molecule absent such a ligand.

In some embodiments, a conjugate or ligand can include a naturally occurring substance or a recombinant or synthetic molecule. Non-limiting examples of conjugates and ligands include serum proteins (e.g., human serum albumin, low-density lipoprotein, globulin), cholesterol moieties, vitamins (e.g., biotin, vitamin E, vitamin B12), folate moieties, steroids, bile acids (e.g., cholic acid), fatty acids (e.g., palmitic acid, myristic acid), carbohydrates (e.g., a dextran, pullulan, chitin, chitosan, inulin, cyclodextrin, hyaluronic acid, or N-acetyl-galactosamine (GalNAc)), glycosides, phospholipids, antibodies or binding fragment thereof (e.g., antibody or binding fragment that targets the siRNA to a specific cell type, such as liver), a dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules (e.g., cholesterol, tocopherol, long fatty acids (e.g., docosanoic, palmitoyl, docosahexaenoic), cholic acid, adamantane acetic acid, 1-pyrene butyric acid, dihydrotestosterone, 1,3-BisO(hexadecyl)glycerol, geranyloxyhexyl group, hexadecylglycerol, borneol, menthol, 1,3-propanediol, heptadecyl group, 03-(oleoyl)lithocholic acid, 03-(oleoyl)cholenic acid, dimethoxytrityl, or phenoxazine), peptides (e.g., antennapedia peptide, Tat peptide, RGD peptides), alkylating agents, polymers, such as polyethylene glycol (PEG) (e.g., PEG-40K), poly amino acids, polyamines (e.g., spermine, spermidine), alkyls, substituted alkyls, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

In some embodiments, the conjugate or ligand comprises a carbohydrate. Carbohydrates include, but are not limited to, sugars (e.g., monosaccharides, disaccharides, trisaccharides, tetrasaccharides, and oligosaccharides containing from about 4, 5, 6, 7, 8, or 9 monosaccharide units) and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units.

In some embodiments, the carbohydrate incorporated into the conjugate or ligand is an amino sugar, such as galactosamine, glucosamine, N-acetyl-galactosamine (GalNAc), and N-acetyl-glucosamine. In some embodiments, the conjugate or ligand comprises N-acetyl-galactosamine and derivatives thereof. Non-limiting examples of GalNAc- or galactose-containing ligands that can be incorporated into the siRNAs of the disclosure are described in WO 2020/243490; WO 2020/097342; WO 2021/119325; PCT/US2021/019629; PCT/US2021/019628; PCT/US2021/021199; Sig. Transduct. Target Ther. 5 (101), 1-25, 2020; ACS Chem. Biol. 10 (5), 1181-1187, 2015; J. Am. Chem. Soc. 136 (49), 16958-16961, 2014; Nucleic Acids Res. 42 (13), 8796-8807, 2014; Molec. Ther. 28 (8), 1759-1771, 2020; and Nucleic Acid Ther. 28 (3), 109-118, 2018, all of which are hereby incorporated herein by reference in their entireties.

The conjugate or ligand can be attached or conjugated to the siRNA molecule directly or indirectly. For instance, in some embodiments, the ligand is covalently attached directly to the sense or antisense strand of the siRNA molecule. In other embodiments, the ligand is covalently attached via a linker to the sense or antisense strand of the siRNA molecule. The ligand can be attached to nucleobases, sugar moieties, or internucleoside linkages of polynucleotides (e.g. sense strand or antisense strand) of the siRNA molecules of the disclosure. In some embodiments, the conjugate or ligand may be attached to the 5′ end and/or to the 3′ end of the sense and/or antisense strand of the siRNA molecule. In certain embodiments, the ligand is covalently attached to the 5′ end of the sense strand. In some embodiments, the ligand is covalently attached to the 3′ end of the sense strand. In some embodiments, the ligand is attached to the 5′ terminal nucleotide of the sense strand or the 3′ terminal nucleotide of the sense strand.

In some embodiments, the conjugate or ligand covalently attached to the sense and/or antisense strand of the siRNA molecule comprises a GalNAc derivative. In some embodiments, the GalNAc derivative is attached to the 5′ end and/or to the 3′ end of the sense and/or antisense strand of the siRNA molecule. In some embodiments, the GalNAc derivative is attached to the 3′ end of the sense strand. In some embodiments, the GalNAc derivative is attached to the 5′ end of the sense strand. In some embodiments, the GalNAc derivative is attached to the 3′ end of the antisense strand. In some embodiments, the GalNAc derivative is attached to the 5′ end of the antisense strand. In some embodiments, the GalNAc derivative is attached to the 5′ end of the sense strand and to the 3′ end of the sense strand.

In some embodiments, the conjugate or ligand is a GalNAc derivative comprising 1, 2, 3, 4, 5, or 6 monomeric GalNAc units. In some embodiments, the conjugate or ligand is a GalNAc derivative comprising 1 monomeric GalNAc units. In some embodiments, the conjugate or ligand is a GalNAc derivative comprising 2 monomeric GalNAc units. In some embodiments, the conjugate or ligand is a GalNAc derivative comprising 3 monomeric GalNAc units. In some embodiments, the conjugate or ligand is a GalNAc derivative comprising 4 monomeric GalNAc units. In some embodiments, the conjugate or ligand is a GalNAc derivative comprising 5 monomeric GalNAc units. In some embodiments, the conjugate or ligand is a GalNAc derivative comprising 6 monomeric GalNAc units. In some embodiments, a various amounts of monomeric GalNAc units are attached at the 5′ end and the 3′ end of the sense strand. In some embodiments, a various amounts of monomeric GalNAc units are attached at the 5′ end and the 3′ end of the antisense strand. In some embodiments, 1, 2, 3, 4, 5, or 6 monomeric GalNAc units are attached at the 5′ end of the sense strand. In some embodiments, 1, 2, 3, 4, 5, or 6 monomeric GalNAc units are attached at the 3′ end of the sense strand. In some embodiments, 1, 2, 3, 4, 5, or 6 monomeric GalNAc units are attached at the 5′ end of the antisense strand. In some embodiments, 1, 2, 3, 4, 5, or 6 monomeric GalNAc units are attached at the 3′ end of the antisense strand. In some embodiments, the same number of monomeric GalNAc units are attached at both the 5′ end and the 3′ end of the sense strand. In some embodiments, the same number of monomeric GalNAc units are attached at both the 5′ end and the 3′ end of the antisense strand. In some embodiments, different number of monomeric GalNAc units are attached at the 5′ end and the 3′ end of the sense strand. In some embodiments, different number of monomeric GalNAc units are attached at the 5′ end and the 3′ end of the antisense strand.

In some embodiments, the double stranded siRNA molecule of any one of siRNA Duplex ID Nos. D1-D515 or MD1-MD673, further comprises a GalNAc derivative attached to the 5′ end and/or to the 3′ end of the sense and/or antisense strand of the siRNA molecule. In some embodiments, the double stranded siRNA molecule selected from any one of the siRNA Duplexes of Table 1 or Table 2 or Table 3 or Table 4 further comprises a GalNAc derivative attached to the 5′ end and/or to the 3′ end of the sense and/or antisense strand of the siRNA molecule.

### PNPLA3

In some embodiments, any of the siRNAs disclosed herein specifically downregulate expression of PNPLA3 gene or a variant thereof. In some embodiments, any of the siRNAs disclosed herein specifically downregulate expression of PNPLA3 gene or a variant thereof in a cell by at least about 10%, 15%, 20%, 25%, 30%, 35%, 40%, 45%, 50%, 55%, 60%, 65%, 70%, 75%, 80%, 85%, 90%, 95%, or 100%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 30%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 50%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 60%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 70%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 75%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 80%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 85%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 90%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 95%, wherein the percent of downregulation of expression is compared to a cell not contacted with the siRNA. In some embodiments, any of the siRNAs disclosed herein specifically downregulate expression of PNPLA3 gene or a variant thereof in a cell by at least about 100%, wherein the percent of downregulation of expression is compared to a cell not contacted with the siRNA.

The expression of PNPLA3 gene is measured by any method known in the art. Exemplary methods for measuring expression of PNPLA3 gene include, but are not limited to, quantitative PCR, RT-PCR, RT-qPCR, western blot, Southern blot, northern blot, FISH, DNA microarray, tiling array, and RNA-Seq. The expression of the PNPLA3 gene may be assessed, for example, based on the level, or the change in the level, of any variable associated with PNPLA3 gene expression, e.g., PNPLA3 mRNA level, PNPLA3 protein level, and/or the number or extent of amyloid deposits. This level may be assessed, for example, in an individual cell or in a group of cells, including, for example, a sample derived from a subject. In some embodiments, downregulation or inhibition may be assessed by a decrease in an absolute or relative level of one or more variables that are associated with PNPLA3 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive or attenuated agent control).

In some embodiments, the PNPLA3 gene comprises a nucleotide sequence that is at least about 80%, 85%, 90%, 91%, 92%, 93%, 94%, 95%, 96%, 97%, 98%, 99%, or 100% identical to the nucleotide sequence of SEQ ID NO: 1 across the full-length of SEQ ID NO: 1 (PNPLA3 wild-type CDS (NCBI Ref. No. NM_025225.3)).

In some embodiments, the PNPLA3 gene comprises a nucleotide sequence having less than or equal to 1, 2, 3, 4, 5, 6, 7, 8, 9, 10, 11, 12, 13, 14, 15, 16, 17, 18, 19, or 20 nucleotide mismatches to the nucleotide sequence of SEQ ID NO: 1 across the full-length of SEQ ID NO: 1. In some embodiments, the PNPLA3 gene comprises a nucleotide sequence having a single nucleotide missense mutation at position 444 of the nucleotide sequence of SEQ ID NO: 1 (i.e., SEQ ID NO: 2067).

In some embodiments, the PNPLA3 gene comprises a nucleotide sequence encoding a PNPLA3 protein having an amino acid sequence that is at least about 80%, 85%, 90%, 91%, 92%, 93%, ₉₄%, 95%, 96%, 97%, 98%, 99%, or 100% identical to the amino acid sequence of SEQ ID NO: 2 across the full-length of SEQ ID NO: 2 (PNPLA3 wild-type protein (NCBI Ref. No. NM_079501.2)).

In some embodiments, the PNPLA3 gene comprises a nucleotide sequence encoding a PNPLA3 protein having an amino acid sequence having less than or equal to 1, 2, 3, 4, 5, 6, 7, 8, 9, 10, 11, 12, 13, 14, 15, 16, 17, 18, 19, or 20 substitutions, deletions, or insertions to the amino acid sequence of SEQ ID NO: 2 across the full-length of SEQ ID NO: 2. In some embodiments, the PNPLA3 gene comprises a nucleotide sequence encoding a PNPLA3 protein having an amino acid sequence having a substitution at position 148 of the amino acid sequence of SEQ ID NO: 2. In some embodiments, the substitution at position 148 is an I148M substitution.

In some embodiments, the fragment of the PNPLA3 gene is about 10 to about 50, or about 15 to about 50, or about 15 to about 45 nucleotides, or about 15 to about 40, or about 15 to about 35, or about 15 to about 30, or about 15 to about 25, or about 17 to about 23 nucleotides, or about 17 to about 22, or about 17 to about 21, or about 18 to about 23, or about 18 to about 22, or about 18 to about 21, or about 19 to about 23, or about 19 to about 22, or about 19 to about 21 nucleotides in length. In some embodiments, the fragment of the PNPLA3 gene spans a region of the PNPLA3 gene containing the nucleotide at position 444 of SEQ ID NO: 1 or spans a region within 100, 200, 300, 400, or 500 nucleotides of position 444 of SEQ ID NO: 1. In some embodiments, the nucleotide at position 444 of SEQ ID NO: 1 contains a C to G substitution (SEQ ID NO: 2067).

In some embodiments, the antisense strand is complementary to the fragment of the PNPLA3 gene containing a C to G substitution at position 444 of SEQ ID NO: 1 (i.e., SEQ ID NO: 2067). In some embodiments, the antisense strand is complementary to the fragment of the PNPLA3 gene that is within 100, 200, 300, 400, or 500 nucleotides of position 444 of SEQ ID NO: 1.

Administration of siRNA

Administration of any of the siRNAs disclosed herein may be conducted by methods known in the art, including as described below. The siRNAs of the present disclosure may be given systemically or locally, for example, orally, nasally, parenterally, topically, intracisternally, intravaginally, or rectally, and are given in forms suitable for each administration route.

The delivery of a siRNA molecule of the disclosure to a cell, e.g., a cell within a subject, such as a human subject (e.g., a subject in need thereof, including a subject having a disease, disorder or condition associated with PNPLA3 gene expression) can be achieved in a number of different ways. For example, in some embodiments, delivery may be performed by contacting a cell with a siRNA of the disclosure either in vitro, in vivo, or ex vivo. In some embodiments, in vivo delivery may be performed, for example, by administering a pharmaceutical composition comprising a siRNA molecule to a subject. In some embodiments, in vivo delivery may be performed by administering one or more vectors that encode and direct the expression of the siRNA.

In general, any method of delivering a nucleic acid molecule (in vitro, in vivo, or ex vivo) can be adapted for use with a siRNA molecule of the disclosure. For in vivo delivery, factors to consider in order to deliver a siRNA molecule include, for example, biological stability of the delivered molecule, prevention of non-specific effects, and accumulation of the delivered molecule in the target tissue and non-target tissue.

In some embodiments, the non-specific effects of a siRNA can be minimized by local administration, for example, by direct injection or implantation into a tissue or topically administering the preparation. Local administration to a treatment site can, for example, maximize the local concentration of the agent, limit the exposure of the agent to systemic tissues that can otherwise be harmed by the agent or that can degrade the agent, and permit a lower total dose of the siRNA molecule to be administered.

In some embodiments, the siRNAs or pharmaceutical compositions comprising the siRNAs of the disclosure can be locally administered to relevant tissues ex vivo, or in vivo through, for example, injection, infusion pump or stent, with or without their incorporation in biopolymers.

For administering a siRNA for the treatment of a disease, the siRNA can be modified or alternatively delivered using a drug delivery system; both methods can act, for example, to prevent the rapid degradation of the dsRNA by endo- and exo-nucleases in vivo. Modification of the siRNA or the pharmaceutical carrier can also permit targeting of the siRNA composition to the target tissue and avoid undesirable off-target effects. For example, siRNA molecules can be modified by conjugation to lipophilic groups such as cholesterol as described above to, e.g., enhance cellular uptake and prevent degradation.

In some embodiments, the siRNA can be delivered using drug delivery systems such as a nanoparticle, a dendrimer, a polymer, liposomes, or a cationic delivery system. Positively charged cationic delivery systems can facilitate binding of a siRNA molecule (negatively charged) and also enhance interactions at the negatively charged cell membrane to permit efficient uptake of a siRNA by the cell. In some embodiments, cationic lipids, dendrimers, or polymers can either be bound to a siRNA, or induced to form a vesicle or micelle that encases a siRNA. The formation of vesicles or micelles may further prevent degradation of the siRNA when administered systemically, for example.

Some non-limiting examples of drug delivery systems useful for systemic delivery of siRNAs include DOTAP, cardiolipin, polyethyleneimine, Arg-Gly-Asp (RGD) peptides, and polyamidoamines. In some embodiments, a siRNA forms a complex with cyclodextrin for systemic administration.

### Pharmaceutical Compositions

The siRNA molecules of the disclosure can be administered to animals, including to mammals, and in particular to humans, as pharmaceuticals by themselves, in mixtures with one another, and/or in the form of pharmaceutical compositions.

The present disclosure includes pharmaceutical compositions and formulations which include the siRNA molecules of the disclosure. In some embodiments, a siRNA molecule of the disclosure may be administered in a pharmaceutical composition. In some embodiments, the pharmaceutical compositions of the disclosure comprise one or more siRNA molecules of the disclosure and a pharmaceutically acceptable carrier. When reference is made in the present disclosure to a siRNA molecule, it is to be understood that reference is also made to a pharmaceutical composition containing the siRNA molecule, if appropriate.

In some embodiments, the pharmaceutical composition comprises at least 1, 2, 3, 4, 5, 6, 7, 8, 9, 10, 11, 12, 13, 14, 15, 16, 17, 18, 19, or 20 or more of any of the siRNA molecules disclosed herein.

In some embodiments, any of the pharmaceutical compositions disclosed herein comprise one or more excipients, carriers, wetting agents, diluents, emulsifiers, lubricants, coloring agents, release agents, coating agents, sweetening, flavoring and perfuming agents, preservatives and antioxidants.

In some embodiments, a siRNA molecule of the disclosure may be administered in “naked” form, where the modified or unmodified siRNA molecule is directly suspended in aqueous or suitable buffer solvent, as a “free siRNA.” The free siRNA may be in a suitable buffer solution, which may comprise, for example, acetate, citrate, prolamine, carbonate, or phosphate, or any combination thereof. In one embodiment, the buffer solution is phosphate buffered saline (PBS). The pH and osmolality of the buffer solution containing the siRNA can be adjusted such that it is suitable for administering to a subject.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In certain embodiments, a pharmaceutical composition of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siRNA molecule) of the present disclosure. In certain embodiments, an aforementioned composition renders orally bioavailable a siRNA molecule of the present disclosure.

Methods of preparing these formulations or pharmaceutical compositions include, for example, the step of bringing into association a siRNA molecule of the present disclosure with the carrier and, optionally, one or more accessory ingredients. In general, the formulations are prepared by uniformly and intimately bringing into association a siRNA molecule of the present disclosure with liquid carriers, or finely divided solid carriers, or both, and then, if necessary, shaping the product.

Administration of the pharmaceutical compositions of the present disclosure may be via any common route, and they are given in forms suitable for each administration route. Such routes include, but are not limited to, parenteral (e.g., subcutaneous, intramuscular, intraperitoneal or intravenous), oral, nasal, airway (e.g., aerosol), buccal, intradermal, transdermal, sublingual, rectal, and vaginal. In some embodiments, administration is by direct injection into liver tissue or delivery through the hepatic portal vein. In some embodiments, the pharmaceutical composition is administered orally. In some embodiments, the pharmaceutical composition is administered parenterally. In some embodiments, the compositions are administered by subcutaneous or intravenous infusion or injection. In some embodiments, the pharmaceutical composition is administered subcutaneously.

Pharmaceutical compositions of the disclosure suitable for oral administration may be, for example, in the form of capsules (e.g., hard or soft capsules), cachets, pills, tablets, lozenges (using a flavored basis, usually, e.g., sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a siRNA molecule of the present disclosure as an active ingredient. A siRNA molecule of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as, for example, sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made, for example, by compression or molding, optionally with one or more accessory ingredients. Compressed tablets may be prepared, for example, using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made, for example, by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried.

They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the siRNA molecules of the disclosure include, for example, pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as, for example, wetting agents, emulsifying and suspending agents, sweetening, flavoring, coloring, perfuming and preservative agents.

Suspensions, in addition to the siRNA molecules, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more siRNA molecules of the disclosure with one or more suitable nonirritating excipients or carriers comprising, for example, cocoa butter, polyethylene glycol, a suppository wax or a salicylate, and which, for example, is solid at room temperature, but liquid at body temperature and, therefore, will melt in the rectum or vaginal cavity and release the siRNA molecule.

Formulations of the present disclosure which are suitable for vaginal administration also include, for example, pessaries, tampons, creams, gels, pastes, foams or spray formulations containing such carriers as are known in the art to be appropriate.

Dosage forms for the topical or transdermal administration of a siRNA molecule of this disclosure include, for example, powders, sprays, ointments, pastes, creams, lotions, gels, solutions, patches and inhalants. The siRNA molecule may be mixed under sterile conditions with a pharmaceutically acceptable carrier, and with any preservatives, buffers, or propellants which may be required.

The ointments, pastes, creams and gels may contain, in addition to an active siRNA molecule of this disclosure, excipients, such as, for example, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a siRNA molecule of this disclosure, excipients such as, for example, lactose, talc, silicic acid, aluminum hydroxide, calcium silicates and polyamide powder, or mixtures of these substances. Sprays can additionally contain customary propellants, such as, for example, chlorofluorohydrocarbons and volatile unsubstituted hydrocarbons, such as butane and propane.

Transdermal patches have the added advantage of providing controlled delivery of a siRNA molecule) of the present disclosure to the body. Such dosage forms can be made by dissolving or dispersing the siRNA molecule in the proper medium. Absorption enhancers can also be used to increase the flux of the siRNA molecule across the skin. The rate of such flux can be controlled, for example, by either providing a rate controlling membrane or dispersing the siRNA molecule in a polymer matrix or gel.

Pharmaceutical compositions of this disclosure suitable for parenteral administration comprise one or more siRNA molecules of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain, for example, sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The pharmaceutical compositions of the disclosure may also contain adjuvants such as preservatives, wetting agents, emulsifying agents and dispersing agents. Prevention of the action of microorganisms upon the subject compounds may be ensured, for example, by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about, for example, by the inclusion of agents which delay absorption such as aluminum monostearate and gelatin.

In some embodiments, in order to prolong the effect of a drug, it is desirable to slow the absorption of the drug, for example from subcutaneous or intramuscular injection. This may be accomplished, for example, by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

In some embodiments, the administration is via a depot injection. Injectable depot forms can be made by forming microencapsule matrices of the subject siRNA molecules in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations can also be prepared, for example, by entrapping the drug in liposomes or microemulsions which are compatible with body tissue.

Depot injection may release the siRNA in a consistent way over a prolonged time period. Thus, a depot injection may reduce the frequency of dosing needed to obtain a desired effect, e.g., a desired inhibition of PNPLA3, or a therapeutic or prophylactic effect. A depot injection may also provide more consistent serum concentrations. Depot injections may include, for example, subcutaneous injections or intramuscular injections. In some embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used, for example, for intravenous, subcutaneous, arterial, or epidural infusions. In some embodiments, the infusion pump is a subcutaneous infusion pump. In other embodiments, the pump is a surgically implanted pump that delivers the siRNA to the subject.

In some embodiments, the pharmaceutical compositions of the disclosure are packaged with or stored within a device for administration. Devices for injectable formulations include, but are not limited to, injection ports, pre-filled syringes, auto injectors, injection pumps, on-body injectors, and injection pens. Devices for aerosolized or powder formulations include, but are not limited to, inhalers, insufflators, aspirators, and the like. Thus, the present disclosure includes administration devices comprising a pharmaceutical composition of the disclosure for treating or preventing one or more of the disorders described herein.

The mode of administration may be chosen, for example, based upon whether local or systemic treatment is desired and based upon the area to be treated. The route and site of administration may be chosen, for example, to enhance targeting.

Regardless of the route of administration selected, the siRNA molecules of the present disclosure, which may be used in a suitable hydrated form, and/or the pharmaceutical compositions of the present disclosure, may be formulated into pharmaceutically-acceptable dosage forms by methods known to those of skill in the art. Methods for the formulation of pharmaceutical compositions depend on a number of criteria, including, but not limited to, route of administration, type and extent of disease or disorder to be treated, and/or dose to be administered. In some embodiments, the pharmaceutical compositions are formulated based on the intended route of delivery. The preparation of the pharmaceutical compositions can be carried out in a known manner. For this purpose, one or more compounds, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage.

The pharmaceutical compositions may conveniently be presented in unit dosage form and may be prepared by any methods known in the art of pharmacy. The amount of active ingredient which can be combined with a carrier material to produce a single dosage form will vary depending upon the host being treated and the particular mode of administration, for example, as described below. The amount of active ingredient which can be combined with a carrier material to produce a single dosage form will generally be, for example, that amount of the siRNA molecule which produces a therapeutic effect. In some embodiments, for example, out of one hundred percent, this amount will range from about 0.1 percent to about ninety-nine percent of active ingredient, or from about 5 percent to about 70 percent, or from about 10 percent to about 30 percent.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient. For example, the siRNA molecules in the pharmaceutical compositions of the disclosure may be administered in dosages sufficient to downregulate the expression of a PNPLA3 gene.

The siRNA molecules and pharmaceutical compositions of the present disclosure may be used to treat a disease in a subject in need thereof, for example in the methods described below.

### Dosages

The dosage amount and/or regimen utilizing a siRNA molecule of the disclosure may be selected in accordance with a variety of factors including, for example, the activity of the particular siRNA molecule of the present disclosure employed, or the salt thereof; the severity of the condition to be treated; the route of administration; the time of administration; the rate of excretion or metabolism of the particular siRNA molecule being employed; the rate and extent of absorption; the duration of the treatment; other drugs, compounds and/or materials used in combination with the particular siRNA molecule employed; the type, species, age, sex, weight, condition, general health and prior medical history of the patient being treated; the renal and hepatic function of the patient; and like factors well known in the medical arts. A consideration of these factors is well within the purview of the ordinarily skilled clinician for the purpose of determining a therapeutically effective amount.

In some embodiments, a suitable daily dose of a siRNA molecule of the disclosure is, for example, the amount of the siRNA molecule that is the lowest dose effective to produce a therapeutic effect. For example, a physician or veterinarian could start doses of the siRNA molecules of the disclosure employed in a pharmaceutical composition at levels lower than that required in order to achieve the desired therapeutic effect and gradually increase the dosage until the desired effect is achieved. Such an effective dose may depend, for example, upon the factors described above. In some embodiments, the siRNA molecules of the disclosure may be administered in dosages sufficient to downregulate or inhibit expression of a PNPLA3 gene.

In some embodiments, the siRNA molecule is administered at about 0.01 mg/kg to about 200 mg/kg, or at about 0.1 mg/kg to about 100 mg/kg, or at about 0.5 mg/kg to about 50 mg/kg. In some embodiments, the siRNA molecule is administered at about 1 mg/kg to about 40 mg/kg, or at about 1 mg/kg to about 30 mg/kg, or at about 1 mg/kg to about 20 mg/kg, or at about 1 mg/kg to about 15 mg/kg, or at about 1 mg/kg to about 10 mg/kg. In some embodiments, the siRNA molecule is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.85, 0.90, 0.95, or 1 mg/kg. In some embodiments, the siRNA molecule is administered at a dose equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In some embodiments, the siRNA molecule is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the siRNA molecule is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

In some embodiments, treatment of a subject with a therapeutically effective amount of a siRNA molecule of the disclosure can include a single treatment or a series of treatments. In some embodiments, the siRNA molecule is administered as a single dose or may be divided into multiple doses. In some embodiments, the effective daily dose of the siRNA molecule may be administered as two, three, four, five, six, seven, eight, nine, ten or more doses or sub-doses administered separately at appropriate intervals throughout the day, optionally, in unit dosage forms.

In some embodiments, the siRNA molecule is administered once daily. In some embodiments, the siRNA molecule is administered once weekly. In some embodiments, the siRNA molecule is administered at least 1, 2, 3, 4, 5, 6, 7, 8, 9, 10, 11, 12, 13, 14, or 15 times per day. In some embodiments, the siRNA molecule is administered at least 1, 2, 3, 4, 5, 6, 7, 8, 9, 10, 11, 12, 13, 14, 15, 16, 17, 18, 19, 20, or 21 times a week. In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, the siRNA molecule is administered once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the siRNA molecule is administered every 3 days. In some embodiments, the siRNA molecule is administered once every 1, 2, 3, 4, 5, 6, 7, 8, 9, 10, 11, 12, 13, 14, or 15 weeks. In some embodiments, the siRNA molecule is administered once a month. In some embodiments, the siRNA molecule is administered once every 1, 2, 3, 4, 5, 6, 7, 8, 9, 10, 11, 12, 13, 14, or 15 months.

In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the siRNA molecule is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the siRNA molecule is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the siRNA molecule is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the siRNA molecule is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

In some embodiments, a repeat-dose regimen may include administration of a therapeutically effective amount of siRNA on a regular basis, such as every other day, once weekly, once per quarter (i.e., about every 3 months), or once a year. In some embodiments, the dosage amount and/or frequency may be decreased after an initial treatment period. In some embodiments, when the siRNA molecules described herein are co-administered with another active agent, the therapeutically effective amount may be less than when the siRNA molecule is used alone.

### Methods and Uses

Disclosed herein are also methods of treating a PNPLA3-associated disease in a subject in need thereof, comprising administering to the subject any of the siRNA molecules and/or pharmaceutical compositions comprising a siRNA molecule disclosed herein. In an embodiment, the PNPLA3-associated disease is a liver disease.

When the siRNA molecules of the present disclosure are administered as pharmaceuticals, to humans and animals, they can be given per se or as a pharmaceutical composition as described above containing, for example, 0.1 to 99% (more preferably, 10 to 30%) of siRNA molecule in combination with a pharmaceutically acceptable carrier.

In some embodiments, a method of treating a disease in a subject in need thereof comprises administering to the subject an amount of any of the siRNA molecules disclosed herein. In an embodiment, the amount is a therapeutically effective amount. In some embodiments, a method of treating a disease in a subject in need thereof comprises administering to the subject an amount of any of the pharmaceutical compositions disclosed herein. In an embodiment, the amount is a therapeutically effective amount.

In some embodiments, a method of treating a disease in a subject in need thereof comprises administering to the subject any of the siRNA molecules or pharmaceutical compositions disclosed herein in combination with an additional active agent. In some embodiments, the additional active agent is a liver disease treatment agent. In an embodiment, the amount of the siRNA molecule is a therapeutically effective amount. In an embodiment, the amount of the additional active agent is a therapeutically effective amount.

In some embodiments, the siRNA molecule and the liver disease treatment agent are administered separately. In some embodiments, the siRNA molecule or pharmaceutical composition and the liver disease treatment agent are administered concurrently. In some embodiments, the siRNA molecule or pharmaceutical composition and the liver disease treatment agent are administered sequentially. In some embodiments, the siRNA molecule or pharmaceutical composition is administered prior to administering the liver disease treatment agent. In some embodiments, the siRNA molecule or pharmaceutical composition is administered after administering the liver disease treatment agent. In some embodiments, the pharmaceutical composition comprises the siRNA and the liver disease treatment agent.

Also disclosed herein are methods of reducing the expression level of PNPLA3 in a subject in need thereof comprising administering to the subject an amount of a siRNA molecule or pharmaceutical composition according to the disclosure. In an embodiment, the amount of the additional active agent is a therapeutically effective amount. In some embodiments, the method of reducing the expression level of PNPLA3 in a subject in need thereof comprising administering to the subject an amount of a siRNA molecule or pharmaceutical composition according to the disclosure reduces the expression level of PNPLA3 in hepatocytes in the subject following administration of the siRNA molecule or pharmaceutical composition as compared to the PNPLA3 expression level in a patient not receiving the siRNA or pharmaceutical composition.

Also disclosed herein are methods of preventing at least one symptom of a liver disease in a subject in need thereof comprising administering to the subject an amount of any of the siRNA molecules or pharmaceutical compositions of the disclosure, thereby preventing at least one symptom of a liver disease in the subject. In an embodiment, the amount of the additional active agent is a therapeutically effective amount.

In another aspect, disclosed herein are uses of any of the siRNA molecules or pharmaceutical compositions of the disclosure in the manufacture of a medicament for treating a liver disease. In some embodiments, the present disclosure provides use of a siRNA molecule of the disclosure or pharmaceutical composition comprising an siRNA of the disclosure that targets a PNPLA3 gene in a cell of a mammal in the manufacture of a medicament for inhibiting expression of the PNPLA3 gene in the mammal.

The methods and uses disclosed herein include administering to a mammal, e.g., a human, a pharmaceutical composition comprising a siRNA molecule that targets a PNPLA3 gene in a cell of the mammal and maintaining for a time sufficient to obtain degradation of the mRNA transcript of the PNPLA3 gene, thereby inhibiting expression of the PNPLA3 gene in the mammal.

The patient or subject of the described methods may be a mammal, and it includes humans and non-human mammals. In some embodiments, the subject is a human, such as an adult human, human teenager, human child, human toddler, or human infant.

The siRNA molecules and/or pharmaceutical compositions of the disclosure can be administered in the disclosed methods and uses by any administration route known in the art, including those described above such as, for example, subcutaneous, intravenous, oral, intraperitoneal, or parenteral routes, including, e.g., intracranial (e.g., intraventricular, intraparenchymal and intrathecal), intramuscular, transdermal, airway (aerosol), nasal, rectal, and topical (including buccal and sublingual) administration.

The siRNA molecules and/or pharmaceutical compositions of the disclosure can be administered in the disclosed methods and uses in any of the of dosages or dosage regimens described above.

### PNPLA3—Associated Diseases

Any of the siRNAs and/or pharmaceutical compositions and/or methods and/or uses disclosed herein may be used to treat a disease, disorder, and/or condition. In some embodiments, the disease, disorder, and/or condition is associated with PNPLA3 expression or activity. In some embodiments, the disease, disorder, and/or condition is a liver disease. As used herein, the term “PNPLA3-associated disease” includes a disease, disorder, or condition that would benefit from a downregulation in PNPLA3 gene expression, replication or activity. Non-limiting examples of PNPLA3-associated diseases include, but are not limited to, fatty liver (steatosis), nonalcoholic steatohepatitis (NASH), cirrhosis of the liver, accumulation of fat in the liver, inflammation of the liver, hepatocellular necrosis, liver fibrosis, obesity, or nonalcoholic fatty liver disease (NAFLD). In an embodiment, the PNPLA3-associated disease is NAFLD. In an embodiments, the PNPLA3-associated disease is NASH. In an embodiment, the PNPLA3-associated disease is fatty liver (steatosis).

### Combination Therapies

Any of the siRNAs or pharmaceutical compositions disclosed herein may be combined with one or more additional active agents in a pharmaceutical composition or in any method according to the disclosure or for use in treating a liver disease. An additional active agent refers to an ingredient with a pharmacologically effect at a relevant dose; an additional active agent may be another siRNA according to the disclosure, a siRNA not in accordance with the disclosure, or a non-siRNA active agent.

In some embodiments, at least 2, 3, 4, 5, 6, 7, 8, 9, 10 or more siRNAs disclosed herein are combined in a combination therapy.

In some embodiments, any of the siRNAs or pharmaceutical compositions disclosed herein are combined with a liver disease treatment agent in a combination therapy. In some embodiments, the liver disease treatment agent is selected from a peroxisome proliferator-activator receptor (PPAR) agonist, farnesoid X receptor (FXR) agonist, lipid-altering agent, incretin-based therapy, and thyroid hormone receptor (THR) modulator.

In some embodiments, any of the siRNAs or pharmaceutical compositions disclosed herein are combined with a PPAR agonist. In some embodiments, the PPAR agonist is selected from a PPARα agonist, dual PPARα/δ agonist, PPARγ agonist, and dual PPARα/γ agonist. In some embodiments, the dual PPARα agonist is a fibrate. In some embodiments, the PPARα/δ agonist is elafibranor. In some embodiments, the PPARγ agonist is a thiazolidinedione (TZD). In some embodiments, TZD is pioglitazone. In some embodiments, the dual PPARα/γ agonist is saroglitazar.

In some embodiments, any of the siRNAs or pharmaceutical compositions disclosed herein are combined with a FXR agonist. In some embodiments, the FXR agonist is selected from obeticholic acis (OCA) and TERN-1010.

In some embodiments, any of the siRNAs or pharmaceutical compositions disclosed herein are combined with a lipid-altering agent. In some embodiments, the lipid-altering agent is aramchol.

In some embodiments, any of the siRNAs or pharmaceutical compositions disclosed herein are combined with an incretin-based therapy. In some embodiments, the incretin-based therapy is a glucagon-like peptide 1 (GLP-1) receptor agonist or dipeptidyl peptidase 4 (DPP-4) inhibitor. In some embodiments, the GLP-1 receptor agonist is exenatide or liraglutide. In some embodiments, the DPP-4 inhibitor is sitagliptin or vildapliptin.

In some embodiments, any of the siRNAs or pharmaceutical compositions disclosed herein are combined with a THR modulator. In some embodiments, the THR modulator is selected from a THR-beta modulator and thyroid hormone analogue. Exemplary THR modulators are described in Jakobsson, et al., Drugs, 2017, 77(15):1613-1621, Saponaro, et al., Front Med (Lausanne), 2020, 7:331, and Kowalik, et al., Front Endocrinol, 2018, 9:382, which are incorporated by reference in their entireties. In some embodiments, the THR-beta modulator is a THR-beta agonist. In some embodiments, the THR-beta agonist is selected from is selected from KB141, sobetirome, Sob-AM2, eprotirome, VK2809, resmetirom, MB07344, IS25, TG68, GC-24 and any one of the compounds disclosed in U.S. Pat. No. 11,091,467, which is incorporated in its entirety herein. In some embodiments, the thyroid hormone analogue is selected from L-94901 and CG-23425.

Generally, the liver disease treatment agent may be used in any combination with the siRNA molecules of the disclosure in a single dosage formulation (e.g., a fixed dose drug combination), or in one or more separate dosage formulations which allows for concurrent or sequential administration of the active agents (co-administration of the separate active agents) to subjects. In some embodiments, the siRNA and the liver disease treatment agent are administered concurrently. In some embodiments, the siRNA and the liver disease treatment agent are administered sequentially. In some embodiments, the siRNA is administered prior to administering the liver disease treatment agent. In some embodiments, the siRNA is administered after administering the liver disease treatment agent. The sequence and frequency in which the siRNA and the liver disease treatment agent are administered can vary. In some embodiments, the siRNA and the liver disease treatment agent are in separate containers. In some embodiments, the siRNA and the liver disease treatment agent are in the same container. In some embodiments, the pharmaceutical composition comprises the siRNA and the liver disease treatment agent. The siRNA and the liver disease treatment agent can be administered by the same route of administration or by different routes of administration.

### EXAMPLES

The following examples are provided to illustrate the present disclosure. Those ordinarily skilled in the art will readily understand that known variations of the following methods, procedures, and/or materials can be used. These examples are provided for the purpose of further illustration and are not intended to be limitations on the disclosure.

Throughout the disclosure, including in the sequences, abbreviations and acronyms may be used with the following meanings unless otherwise indicated:

| Abbreviation(s) | Reagent |
|---|---|
| A | Adenosine |
| C | Cytidine |
| G | Guanosine |
| U | Uridine |
| fX | 2′-fluoro on X where X is A, C, G, or U |
| mX | 2′-O-methyl on X where X is A, C, G, or |
| | U |
| ps | phosphorothioate internucleoside linkage |
| v | vinyl phosphonate |
| EC50 | half-maximal effective concentration |
| GalNAc | N-acetylgalactosamine (including |
| | variations thereof, such as GalNAc4) |
| PD | pharmacodynamics |
| PK | pharmacokinetics |
| PNPLA3 | Patatin-like phospholipase domain- |
| | containing protein 3 gene, including |
| | variants thereof as described herein |
| RT-qPCR | reverse transcriptase-quantitative |
| | polymerase chain reaction |
| DMF | Dimethylformamide |
| AcSK | Acesulfame potassium |
| TBAI | Tetra-n-butylammonium iodide |
| H₂O | Water |
| EA/EtOAc | Ethyl acetate |
| Na₂SO₄ | Sodium sulfate |
| CDCl₃ | Deuterated chloroform |
| CH₃CN/ACN/MeCN | Acetonitrile |
| MeOH | Methanol |
| NaOH | Sodium hydroxide |
| Ar | Argon gas |
| HCl | Hydrochloric acid |
| i-Pr₂O | Diisopropyl ether |
| THF | Tetrahydrofuran |
| LiBr | Lithium bromide |
| DIEA/DIPEA | N,N-Diisopropylethylamine |
| Pd/C | Palladium metal on carbon support |
| N₂ | Nitrogen gas |
| H₂ | Hydrogen gas |
| CD₃CN | Deuterated acetonitrile |
| TBAF | Tetra-n-butylammonium fluoride |
| DCM/CH₂Cl₂ | Dichloromethane |
| MS | Molecular sieves |
| NaHCO₃ | Sodium bicarbonate |
| NH₄HCO₃ | Ammonium bicarbonate |
| iPrOH/iPr-OH/IPA | Isopropanol |
| TEA | Triethanolamine |
| PPh₃ | Triphenylphosphine |
| DIAD | Diisopropyl azodicarboxylate |
| EtOH | Ethanol |
| NH2NH2•H2O | Hydrazine monohydrate |
| DMSO-d₆ | Deuterated dimethyl sulfoxide |
| Py/Pyr | Pyridine |
| MsCl | Methanesulfonyl chloride |
| PE | Petroleum ether |
| CH₃COOH/AcOH | Acetic acid |
| SiO₂ | Silica/Silicone dioxide |
| I₂ | Iodine |
| Na₂S₂O₃ | Sodium thiosulfate |
| AgNO₃ | Silver nitrate |
| DMTCl/DMTrCl | 4,4′-dimethoxytrityl chloride |
| DTT | Dithiothreitol |
| LiOH•H₂O | Lithium hydroxide monohydrate |
| DCI | 1,1′-Carbonyldiimidazole |
| TEMPO | (2,2,6,6-Tetramethylpiperidin-1-yl)oxyl |
| DIB | Diisobutylene |
| SOCl₂ | Thionyl chloride |
| CD₃OD | Deuterated methanol |
| NaBD₄ | Sodium borodeuteride |
| TBSCl | Tert-butyldimethylsilyl chloride |
| Et3SiH | Triethylsilane |
| TFA | Trifluoroacetic acid |
| NH₃•H₂O/NH₃*H₂O | Ammonia |
| FA/HCOOH/HCO₂H | Formic acid |
| BTT | Benzyl-thio-tetrazole |
| DDTT | 3-[(Dimethylaminomethylene)amino]- |
| | 3H-1,2,4-dithiazole-5-thione |
| K₂CO₃ | Potassium carbonate |
| NaH₂PO₄ | Monosodium phosphate |
| NaBr | Sodium bromide |
| KSAc | Potassium thioacetate |
| LiAlH₄ | Lithium aluminium hydride |
| DMSO | Dimethyl sulfoxide |
| CEOP[N(iPr)₂]₂/CEP | 2-Cyanoethyl N,N- |
| [N(iPr)₂]₂/CEP/CEPCl | diisopropylchlorophosphoramidite |
| (CD₃O)₂Mg | Deuterated magnesium methoxide or d6- |
| | magnesium methoxide |
| NH₄Cl | Ammonium chloride |
| ACN-d₃ | Deuterated acetonitrile |
| D₂O | Heavy water/deuterium oxide |
| PDC | Pyridinium dichromate |
| Ac₂O | Acetic anhydride |
| MeOD | Monodeuterated methanol |
| CH₃COOD | Monodeuteroacetic acid |
| DCA | Dichloroacetic acid |
| TES | 2-{[1,3-Dihydroxy-2- |
| | (hydroxymethyl)propan-2- |
| | yl]amino}ethane-1-sulfonic acid |
| DMAP | 4-Dimethylaminopyridine |
| TPSCl | Triphenylsilyl chloride |
| BzCl | Benzoyl chloride |
| DMTrSH | 4,4′-Dimethoxytrityl thiol |
| NaOMe | Sodium methoxide |
| EDCI | 1-Ethyl-3-(3- |
| | dimethylaminopropyl)carbodiimide |
| POM | Polyoxymethylene |
| KOH | Potassium hydroxide |
| NaCl | Sodium chloride |
| iBuCl | Isobutyryl chloride |
| DAIB | (Diacetoxyiodo)benzene |
| NaI | Sodium iodide |
| Boc | Tert-butyloxycarbonyl |
| TMG | Tetramethylguanidine |
| TMSCHN₂ | Trimethylsilyldiazomethane |
| IBX | 2-Iodoxybenzoic acid |
| PivCl | Pivaloyl chloride/chloromethyl pivalate |
| NaH | Sodium hydride |
| CD₃I | Iodomethane-d₃ |
| BSA | Bis(trimethylsilyl)acetamide |
| TMSOTf | Trimethylsilyl trifluoromethanesulfonate |
| CH₃NH₂ | Methylamine |
| DPC | 1,5-Diphenylcarbazide |
| TrtCl/TrCl | Trityl chloride |
| DAST | Diethylaminosulfur trifluoride |
| Tf-Cl/TfCl | Trifluoromethanesulfonyl chloride |
| Et₃N | Triethylamine |
| KOAc | Potassium acetate |
| DABCO | 1,4-Diazabicyclo[2.2.2]octane |
| NaOAc | Sodium acetate |
| n-BuLi | n-Butyl lithium |
| BF₃•OEt₂ | Boron trifluoride etherate |
| BCl₃ | Boron trichloride/trichloroborane |
| NaN₃ | Sodium azide |
| DBU | 1,8-Diazabicyclo[5.4.0]undec-7-ene |
| NH₄F | Ammonium fluoride |
| (COCl)₂ | Oxalyl dichloride |
| MeNH₂ | Methylamine |
| Rh₂(OAc)₄ | Rhodium (II) acetate |
| Boc₂O | Di-tert-butyl dicarbonate |
| PPTS | Pyridinium p-toluenesulfonate |
| Ms₂O | Methanesulfonic anhydride |
| NaBH₄ | Sodium borohydride |
| PhCO2K | Potassium benzoate |
| p-TsOH/TsOH | p-Toluenesulfonic acid |
| NH₃ | Ammonia |
| TBDPSCl | tert-Butyldiphenylsilyl chloride |
| NaIO₄ | Sodium periodate |
| BAIB | (Diacetoxyiodo)benzene |
| Pb(OAc)₄ | Lead (IV) tetraacetate |
| MgSO₄ | Magnesium sulfate |
| CO₂ | Carbon dioxide |
| H₂O₂ | Hydrogen peroxide |
| CaCO₃ | Calcium carbonate |
| DIBAL-H | Diisobutylaluminum hydride |
| CuSO₄ | Copper (II) sulfate |
| CH₃I | Iodomethane |
| Ag₂O | Silver oxide |
| SnCl₄ | Tin (IV) chloride |
| MMTrCl | 4-Methoxytrityl chloride |
| Et₃Si | Triethylsilane |
| NaNO₂ | Sodium nitrite |
| TMSCl | Trimethylsilyl chloride |
| PacCl | Phenoxyacetyl chloride |
| BOMCl | Benzyl chloromethyl ether |
| DCE | Ethylene dichloride |
| t-BuOH | T-butyl alcohol |
| P₂O₅ | Phosphorus pentoxide |
| ETT | 5-Ethylthio-1H-tetrazole |
| AMA | Ammonia methylamine |

### Example 1. siNA Synthesis

This example describes an exemplary method for synthesizing ds-siNAs.

The 2′-OMe phosphoramidite 5′-O-DMT-deoxy Adenosine (NH-Bz), 3′-O-(2-cyanoethyl-N,N-diisopropyl phosphoramidite, 5′-O-DMT-deoxy Guanosine (NH-ibu), 3′-O-(2-cyanoethyl-N,N-diisopropyl phosphoramidite, 5′-O-DMT-deoxy Cytosine (NH-Bz), 3′-O-(2-cyanoethyl-N,N-diisopropyl phosphoramidite, 5′-O-DMT-Uridine 3′-O-(2-cyanoethyl-N,N-diisopropyl phosphoramidite were purchased from Thermo Fisher Milwaukee WI, USA.

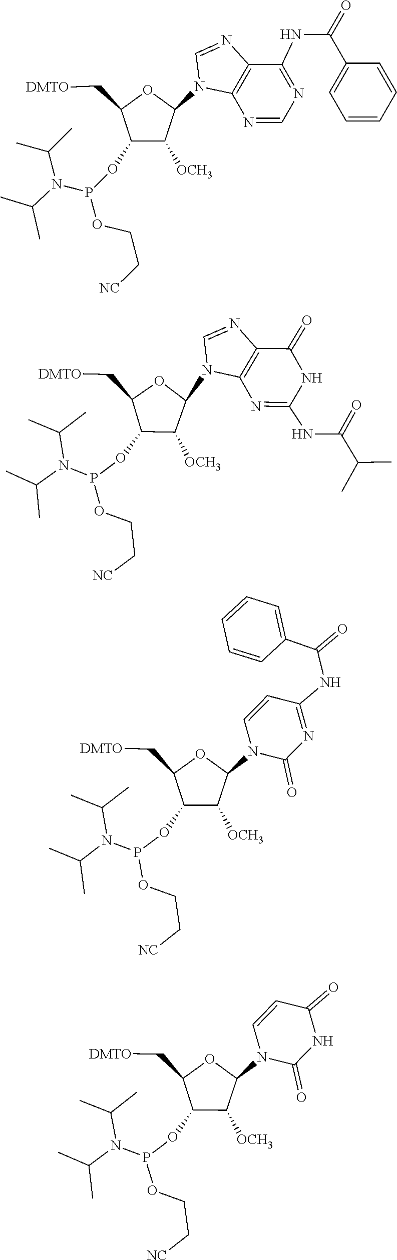

The 2′-F-5′-O-DMT-(NH-Bz) Adenosine-3′-O-(2-cyanoethyl-N,N-diisopropyl phosphoramidite, 2′-F-5′-O-DMT-(NH-ibu)-Guanosine, 3′-O-(2-cyanoethyl-N,N-diisopropyl phosphoramidite, 5′-O-DMT-(NH-Bz)-Cytosine, 2′-F-3′-O-(2-cyanoethyl-N,N-diisopropyl phosphoramidite, 5′-O-DMT-Uridine, 2′-F-3′-O-(2-cyanoethyl-N,N-diisopropyl phosphoramidite were purchased from Thermo Fisher Milwaukee WI, USA.

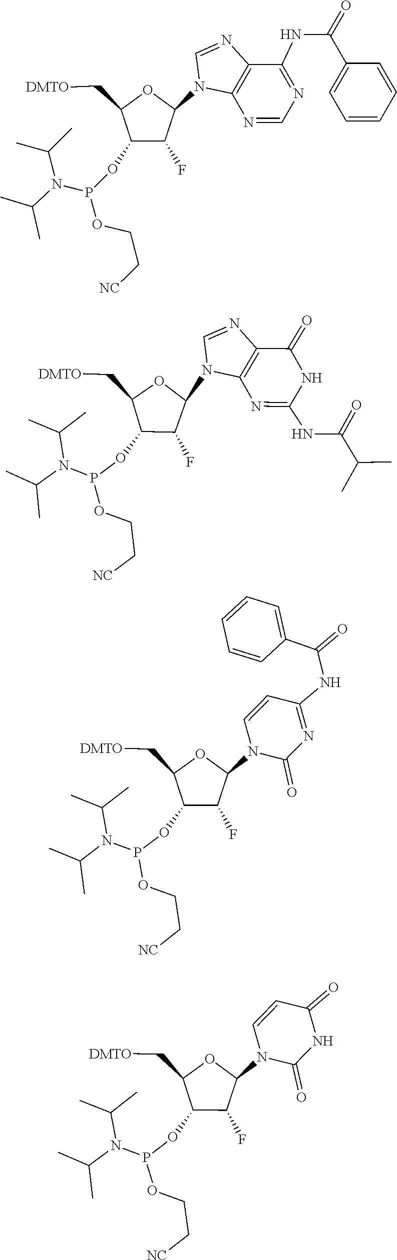

All the monomers were dried in vacuum desiccator with desiccants (P₂O₅, RT 24 h). The solid supports (CPG) attached to the nucleosides and universal supports were obtained from LGC and Chemgenes. The chemicals and solvents for post synthesis workflow were purchased from commercially available sources like VWR/Sigma and used without any purification or treatment. Solvent (Acetonitrile) and solutions (amidite and activator) were stored over molecular sieves during synthesis.

The oligonucleotides were synthesized on DNA/RNA Synthesizers (Expedite 8909 or ABI-394 or MM-48) using standard oligonucleotide phosphoramidite chemistry starting from the 3′ residue of the oligonucleotide preloaded on CPG support. An extended coupling of 0.1M solution of phosphoramidite in CH₃CN in the presence of 5-(ethylthio)-1H-tetrazole activator to a solid bound oligonucleotide followed by standard capping, oxidation and deprotection afforded modified oligonucleotides. The 0.1M 12, THF:Pyridine;Water-7:2:1 was used as oxidizing agent while DDTT ((dimethylamino-methylidene)amino)-3H-1,2,4-dithiazaoline-3-thione was used as the sulfur-transfer agent for the synthesis of oligoribonucleotide phosphorothioates. The stepwise coupling efficiency of all modified phosphoramidites was more than 98%.

| Reagents | Detailed Description |
|---|---|
| Deblock Solution | 3% Dichloroacetic acid (DCA) in |
| | Dichloromethane (DCM) |
| Amidite Concentration | 0.1M in Anhydrous Acetonitrile |
| Activator | 0.25M Ethyl-thio-Tetrazole (ETT) |
| Cap-A solution | Acetic anhydride in Pyridine/THF |
| Cap-B Solution | 16% 1-Methylimidazole in THF |
| Oxidizing Solution | 0.02M I₂, THF:Pyridine; Water-7:2:1 |
| Sulfurizing Solution | 0.2M DDTT in Pyridine/Acetonitrile 1:1 |

### Cleavage and Deprotection:

Deprotection and cleavage from the solid support was achieved with mixture of ammonia methylamine (1:1, AMA) for 15 min at 65° C. When the universal linker was used, the deprotection was left for 90 min at 65° C. or solid supports were heated with aqueous ammonia (28%) solution at 55° C. for 8-16 h to deprotect the base labile protecting groups.

Quantitation of Crude siNA

Samples were dissolved in deionized water (1.0 mL) and quantitated as follows: blanking was first performed with water alone (2 ul) on Thermo Scientific™ Nanodrop UV spectrophotometer or BioTek™ Epoch™ plate reader then oligo sample reading was obtained at 260 nm. The crude material is dried down and stored at −20° C.

### Crude HPLC/LC-MS Analysis

The 0.1 OD of the crude samples were analyzed by HPLC and LC-MS. After confirming the crude LC-MS data then purification step was performed if needed based on the purity.

### HPLC Purification

The unconjugated and GalNAc modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.0 M NaBr, pH 8.5 (buffer B). Fractions containing full-length oligonucleotides were pooled.

Desalting of Purified siNA

The purified dry siNA was then desalted using Sephadex G-25 M (Amersham Biosciences). The cartridge was conditioned with 10 mL of deionized water thrice. Finally, the purified siNA dissolved thoroughly in 2.5 mL RNAse free water was applied to the cartridge drop wise. The salt free siNA was eluted with 3.5 mL deionized water directly into a screw cap vial. Alternatively, some unconjugated siNA was deslated using Pall AcroPrep™ 3K MWCO desalting plates.

### IEX HPLC and Electrospray LC/MS Analysis

Approximately 0.10 OD of siNA was dissolved in water and then pipetted into HPLC autosampler vials for IEX-HPLC and LC/MS analysis. Analytical HPLC and ES LC-MS confirmed the identity and purity of the compounds.

### Duplex Preparation:

Single strand oligonucleotides (Sense and Antisense strands) were annealed (1:1 by molar equivalents, heat at 90° C. for 2 min followed by gradual cooling at room temperature) to give the duplex ds-siNA. The final compounds were analyzed on size exclusion chromatography (SEC).

### Example 2: Synthesis of 5′ End Cap Monomer

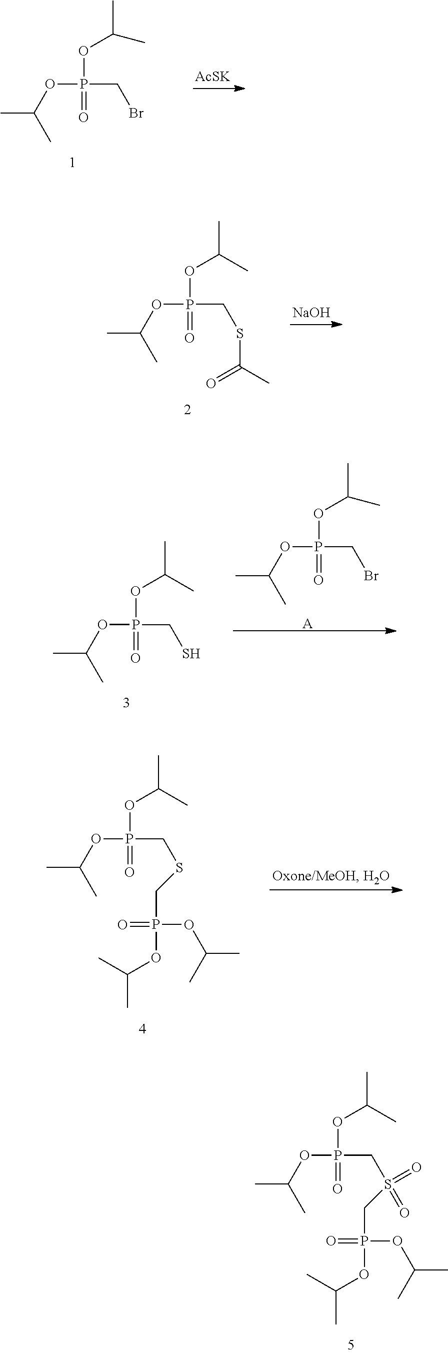

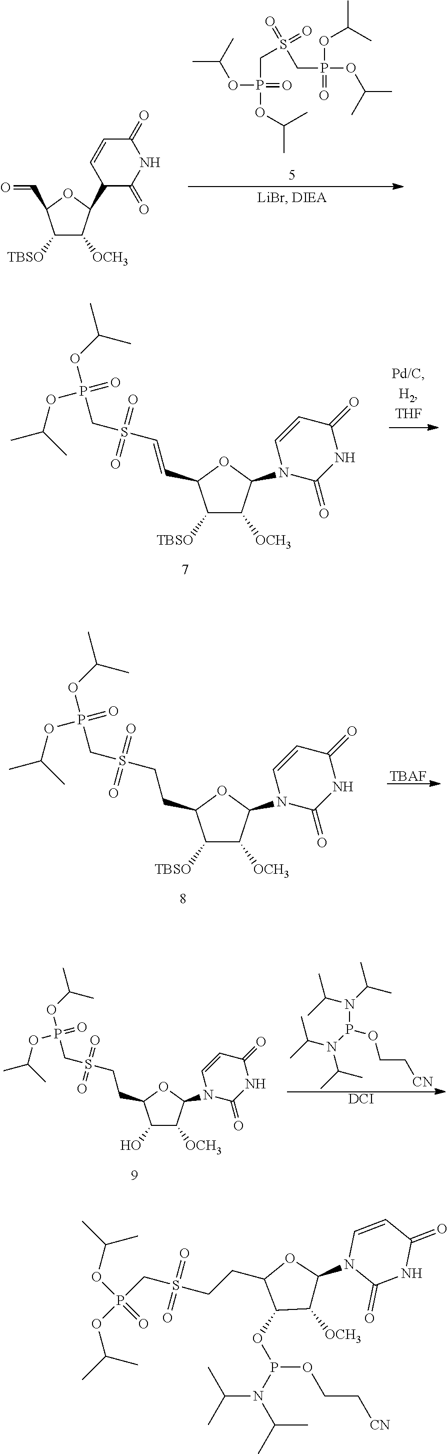

### Example 2 Monomer

### Example 2 Monomer Synthesis Scheme

Preparation of (2): To a solution of 1 (15 g, 57.90 mmol) in DMF (150 mL) were added AcSK (11.24 g, 98.43 mmol) and TBAI (1.07 g, 2.89 mmol), and the mixture was stirred at 25° C. for 12 h. Upon completion as monitored by LCMS, the mixture was diluted with H2O (10 mL) and extracted with EA (200 mL*3). The combined organic layers were washed with brine (200 mL*3), dried over anhydrous Na₂SO₄, filtered and concentrated under reduced pressure to give 2 (14.5 g, 96.52% yield, 98% purity) as a colorless oil. ESI-LCMS: 254.28 [M+H]⁺; ¹H NMR (400 MHz, CDCl₃) δ=4.78-4.65 (m, 2H), 3.19 (d, J=14.1 Hz, 2H), 2.38 (s, 3H), 1.32 (t, J=6.7 Hz, 12H); 31p NMR (162 MHz, CDCl₃) δ=20.59.

Preparation of (3): To a solution of 2 (14.5 g, 57.02 mmol) in CH₃CN (50 mL) and MeOH (25 mL) was added NaOH (3 M, 28.51 mL), and the mixture was stirred at 25° C. for 12 h under Ar. Upon completion as monitored by TLC, the reaction mixture was concentrated under reduced pressure to remove CH₃CN and CH₃OH. The residue was diluted with water (50 mL) and adjust pH=7 by 6M HCl, and the mixture was extracted with EA (50 mL*3). The combined organic layers were washed with brine (50 mL*3), dried over anhydrous Na₂SO₄, filtered and concentrated under reduced pressure to give 3 (12.1 g, crude) as a colorless oil.

Preparation of (4): To a solution of 3 (12.1 g, 57.01 mmol) in CH₃CN (25 mL) and MeOH (25 mL) was added A (14.77 g, 57.01 mmol) dropwise at 25° C., and the mixture was stirred at 25° C. under Ar for 12 h. Upon completion as monitored by LCMS, the reaction mixture was concentrated under reduced pressure to give 4 (19.5 g, 78.85% yield) as a colorless oil. ¹H NMR (400 MHz, CDCl₃) δ=4.80-4.66 (m, 4H), 2.93 (d, J=11.3 Hz, 4H), 1.31 (dd, J=3.9, 6.1 Hz, 24H); ³¹P NMR (162 MHz, CDCl₃) δ=22.18.

Preparation of (5): To a solution of 4 (19.5 g, 49.95 mmol) in MeOH (100 mL) and H2O (100 mL) was added Oxone (61.41 g, 99.89 mmol) at 25° C. in portions, and the mixture was stirred at 25° C. for 12 h under Ar. Upon completion as monitored by LCMS, the reaction mixture was filtered, and the filtrate was concentrated under reduced pressure to remove MeOH. The residue was extracted with EA (50 mL*3). The combined organic layers were washed with brine (50 mL*3), dried over anhydrous Na₂SO₄, filtered and concentrated under reduced pressure to give a residue. The crude product was triturated with i-Pr₂O and n-Hexane (1:2, 100 mL) at 25° C. for 30 min to give 5 (15.6 g, 73.94% yield) as a white solid. ¹H NMR (400 MHz, CDCl₃) δ=4.92-4.76 (m, 4H), 4.09 (d, J=16.1 Hz, 4H), 1.37 (dd, J=3.5, 6.3 Hz, 24H); ³¹P NMR (162 MHz, CDCl₃) δ=10.17.

Preparation of (7): To a mixture of 5 (6.84 g, 16.20 mmol) in THF (20 mL) was added LiBr (937.67 mg, 10.80 mmol) until dissolved, followed by DIEA (1.40 g, 10.80 mmol, 1.88 mL) under argon at 15° C. The mixture was stirred at 15° C. for 15 min. 6 (4 g, 10.80 mmol) were added. The mixture was stirred at 15° C. for 3 h. Upon completion as monitored by LCMS, the reaction mixture was quenched by addition of H₂O (40 mL) and extracted with EA (40 mL*3). The combined organic layers were washed with brine (100 mL), dried over Na₂SO₄, filtered and concentrated under reduced pressure to give a residue. The residue was purified by flash reverse-phase chromatography (120 g C-18 Column, Eluent of 0˜60% ACN/H₂O gradient @80 mL/min) to give 7 (5.7 g, 61.95% yield) as a colorless oil. ESI-LCMS: 611.2 [M+H]⁺; ¹H NMR (400 MHz, CDCl₃); δ=9.26 (s, 1H), 7.50 (d, J=8.1 Hz, 1H), 7.01 (s, 2H), 5.95 (d, J=2.7 Hz, 1H), 5.80 (dd, J=2.1, 8.2 Hz, 1H), 4.89-4.72 (m, 2H), 4.66 (d, J=7.2 Hz, 1H), 4.09-4.04 (m, 1H), 3.77 (dd, J=2.7, 4.9 Hz, 1H), 3.62 (d, J=3.1 Hz, 1H), 3.58 (d, J=3.1 Hz, 1H), 3.52 (s, 3H), 1.36 (td, J=1.7, 6.1 Hz, 12H), 0.92 (s, 9H), 0.12 (s, 6H); ³¹P NMR (162 MHz, CDCl₃) δ=9.02

Preparation of (8): To a mixture of 7 (5.4 g, 8.84 mmol) in THF (80 mL) was added Pd/C (5.4 g, 10% purity) under N₂. The suspension was degassed under vacuum and purged with H₂ several times. The mixture was stirred under H₂ (15 psi) at 20° C. for 1 hr. Upon completion as monitored by LCMS, the reaction mixture was filtered, and the filtrate was concentrated to give 8 (5.12 g, 94.5% yield) as a white solid. ESI-LCMS: 613.3 [M+H]⁺; H NMR (400 MHz, CD₃CN) δ=9.31 (s, 1H), 7.37 (d, J=8.0 Hz, 1H), 5.80-5.69 (m, 2H), 4.87-4.75 (m, 2H), 4.11-4.00 (m, 1H), 3.93-3.85 (m, 1H), 3.80-3.74 (m, 1H), 3.66-3.60 (m, 1H), 3.57-3.52 (m, 1H), 3.49 (s, 3H), 3.46-3.38 (m, 1H), 2.35-2.24 (m, 1H), 2.16-2.03 (m, 1H), 1.89-1.80 (m, 1H), 1.37-1.34 (m, 12H), 0.90 (s, 9H), 0.09 (s, 6H); ³¹P NMR (162 MHz, CD₃CN) δ=9.41.

Preparation of (9): To a solution of 8 (4.4 g, 7.18 mmol) in THF (7.2 mL) was added TBAF (1 M, 7.18 mL), and the mixture was stirred at 20° C. for 1 hr. Upon completion as monitored by LCMS, the reaction mixture was diluted with H₂O (50 mL) and extracted with EA (50 mL*4). The combined organic layers were washed with brine (50 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜5%, MeOH/DCM gradient @40 mL/min) to give 9 (3.2 g, 88.50% yield) as a white solid. ESI-LCMS: 499.2 [M+H]⁺¹; ¹H NMR (400 MHz, CD₃CN) δ=9.21 (s, 1H), 7.36 (d, J=8.3 Hz, 1H), 5.81-5.72 (m, 2H), 4.88-4.74 (m, 2H), 3.99-3.87 (m, 2H), 3.84 (dd, J=1.9, 5.4 Hz, 1H), 3.66-3.47 (m, 7H), 2.98 (s, 1H), 2.44-2.15 (m, 2H), 1.36 (d, J=6.0 Hz, 12H); ³¹P NMR (162 MHz, CD₃CN) δ=9.48.

Preparation of (Example 2 monomer): To a mixture of 9 (3.4 g, 6.82 mmol, 1 eq) and 4A MS (3.4 g) in MeCN (50 mL) was added P1 (2.67 g, 8.87 mmol, 2.82 mL, 1.3 eq) at 0° C., followed by addition of 1H-imidazole-4,5-dicarbonitrile (886.05 mg, 7.50 mmol) at 0° C. The mixture was stirred at 20° C. for 2 h. Upon completion as monitored by LCMS, the reaction mixture was quenched by addition of saturated aq·NaHCO₃ (50 mL) and diluted with DCM (100 mL). The organic layer was washed with saturated aq·NaHCO₃ (50 mL*2), dried over Na₂SO₄, filtered and concentrated under reduced pressure to give a residue. The residue was purified by prep-HPLC: column: YMC-Triart Prep C18 250*50 mm*10 um; mobile phase: [water (10 mM NH₄HCO₃)-ACN]; B %: 15% to give a impure product. The impure product was further purified by a flash silica gel column (0% to 5% i-PrOH in DCM with 0.5% TEA) to give Example 2 monomer (2.1 g, 43.18% yield) as a white solid. ESI-LCMS: 721.2 [M+Na]⁺: H NMR (400 MHz, CD₃CN) δ=9.29 (s, 1H), 7.45 (d, J=8.1 Hz, 1H), 5.81 (d, J=4.2 Hz, 1H), 5.65 (d, J=8.1 Hz, 1H), 4.79-4.67 (m, 2H), 4.26-4.05 (m, 2H), 4.00-3.94 (m, 1H), 3.89-3.63 (m, 6H), 3.53-3.33 (m, 5H), 2.77-2.61 (m, 2H), 2.31-2.21 (m, 1H), 2.16-2.07 (m, 1H), 1.33-1.28 (m, 12H), 1.22-1.16 (m, 1H), 1.22-1.16 (m, 11H); ³¹P NMR (162 MHz, CD₃CN) δ=149.89, 149.78, 10.07, 10.02.

### Example 3. Synthesis of 5′ End Cap Monomer

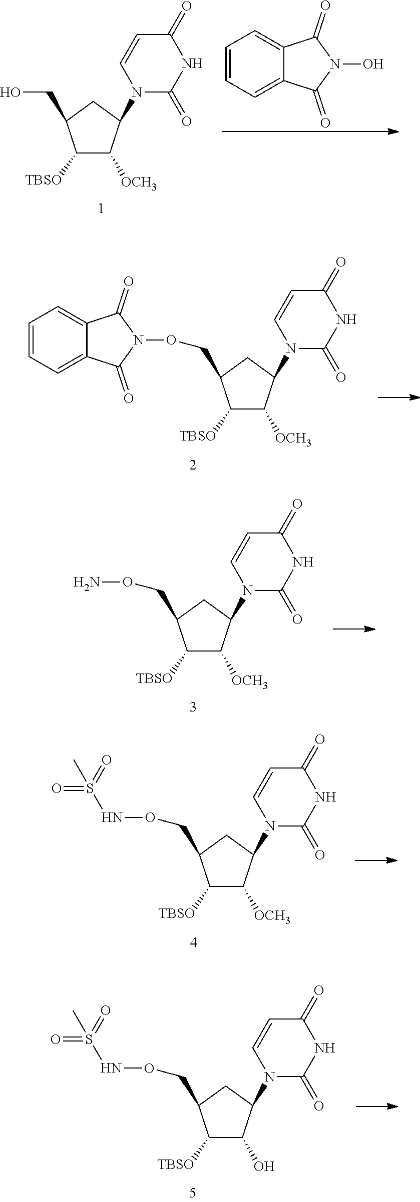

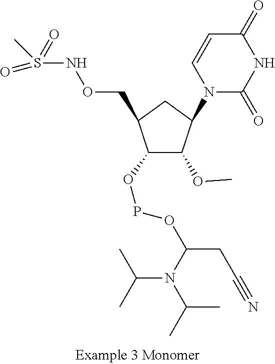

### Example 3 Monomer Synthesis Scheme

Preparation of (2): To a solution of 1 (5 g, 13.42 mmol) in DMF (50 mL) were added PPh₃ (4.58 g, 17.45 mmol) and 2-hydroxyisoindoline-1,3-dione (2.85 g, 17.45 mmol), followed by a solution of DIAD (4.07 g, 20.13 mmol, 3.91 mL) in DMF (10 mL) dropwise at 15° C. The resulting solution was stirred at 15° C. for 18 hr. The reaction mixture was then diluted with DCM (50 mL), washed with H₂O (60 mL*3) and brine (30 mL), dried over Na₂SO₄, filtered and evaporated to give a residue. The residue was then triturated with EtOH (55 mL) for 30 min, and the collected white powder was washed with EtOH (10 mL*2) and dried to give 2 (12.2 g, 85.16% yield) as a white powder (the reaction was set up in two batches and combined) ESI-LCMS: 518.1 [M+H]⁺.

Preparation of (3): 2 (6 g, 11.59 mmol) was suspended in MeOH (50 mL), and then NH₂NH₂·H₂O (3.48 g, 34.74 mmol, 3.38 mL, 50% purity) was added dropwise at 20° C. The reaction mixture was stirred at 20° C. for 4 hr. Upon completion, the reaction mixture was diluted with EA (20 mL) and washed with NaHCO₃ (10 mL*2) and brine (10 mL). The combined organic layers were then dried over Na₂SO₄, filtered and evaporated to give 3 (8.3 g, 92.5% yield) as a white powder. (The reaction was set up in two batches and combined). ESI-LCMS: 388.0 [M+H]⁺; ¹H NMR (400 MHz, DMSO-d₆) δ=11.39 (br s, 1H), 7.72 (d, J=8.1 Hz, 1H), 6.24-6.09 (m, 2H), 5.80 (d, J=4.9 Hz, 1H), 5.67 (d, J=8.1 Hz, 1H), 4.26 (t, J=4.9 Hz, 1H), 4.03-3.89 (m, 1H), 3.87-3.66 (m, 3H), 3.33 (s, 3H), 0.88 (s, 9H), 0.09 (d, J=1.3 Hz, 6H)

Preparation of (4): To a solution of 3 (7 g, 18.06 mmol) and Py (1.43 g, 18.06 mmol, 1.46 mL) in DCM (130 mL) was added a solution of MsCl (2.48 g, 21.68 mmol, 1.68 mL) in DCM (50 mL) dropwise at −78° C. under N₂. The reaction mixture was allowed to warm to 15° C. in 30 min and stirred at 15° C. for 3 h. The reaction mixture was quenched by addition of ice-water (70 mL) at 0° C., and then extracted with DCM (50 mL*3). The combined organic layers were washed with saturated aq·NaHCO₃ (50 mL) and brine (30 mL), dried over Na₂SO₄, filtered and concentrated under reduced pressure to give a residue. The residue was purified by flash silica gel chromatography (ISCO®; 30 g SepaFlash® Silica Flash Column, Eluent of 0˜20% i-PrOH/DCM gradient @30 mL/min to give 4 (6.9 g, 77.94% yield) as a white solid. ESI-LCMS: 466.1 [M+H]⁺; ¹H NMR (400 MHz, DMSO-d₆) δ=11.41 (br s, 1H), 10.15 (s, 1H), 7.69 (d, J=8.1 Hz, 1H), 5.80 (d, J=4.4 Hz, 1H), 5.65 (d, J=8.1 Hz, 1H), 4.24 (t, J=5.2 Hz, 1H), 4.16-3.98 (m, 3H), 3.87 (t, J=4.8 Hz, 1H), 3.00 (s, 3H), 2.07 (s, 3H), 0.88 (s, 9H), 0.10 (d, J=1.5 Hz, 6H)

Preparation of (5): To a solution of 4 (6.9 g, 14.82 mmol) in THF (70 mL) was added TBAF (1 M, 16.30 mL) at 15° C. The reaction mixture was stirred at 15° C. for 18 hr, and then evaporated to give a residue. The residue was purified by flash silica gel chromatography (ISCO®; 24 g SepaFlash® Silica Flash Column, Eluent of 0˜9% MeOH/Ethyl acetate gradient @30 mL/min) to give 5 (1.8 g, 50.8% yield) as a white solid. ESI-LCMS: 352.0 [M+H]⁺; ¹H NMR (400 MHz, DMSO-d₆) δ=11.40 (s, 1H), 10.13 (s, 1H), 7.66 (d, J=8.1 Hz, 1H), 5.83 (d, J=4.9 Hz, 1H), 5.65 (dd, J=1.8, 8.1 Hz, 1H), 5.36 (d, J=6. 2 Hz, 1H), 4.13-4.00 (m, 4H), 3.82 (t, J=5.1 Hz, 1H), 3.36 (s, 3H), 3.00 (s, 3H)

Preparation of (Example 3 monomer): To a mixture of 5 (3 g, 8.54 mmol) and DIEA (2.21 g, 17.08 mmol, 2.97 mL) in ACN (90 mL) was added CEPCl (3.03 g, 12.81 mmol) dropwise at 15° C. The reaction mixture was stirred at 15° C. for 5 h. Upon completion, the reaction mixture was diluted with EA (40 mL) and quenched with 5% NaHCO₃ (20 mL). The organic layer was washed with brine (30 mL), dried over Na₂SO₄, filtered and evaporated to give a residue. The residue was purified by flash silica gel chromatography (ISCO®; 12 g SepaFlash® Silica Flash Column, Eluent of 0˜15% i-PrOH/(DCM with 2% TEA) gradient @20 mL/min) to Example 3 monomer (2.1 g, 43.93% yield) as a white solid. ESI-LCMS: 552.3 [M+H]⁺; ¹H NMR (400 MHz, CD₃CN) δ=8.78 (br s, 1H), 7.57 (dd, J=4.6, 8.2 Hz, 1H), 5.97-5.80 (m, 1H), 5.67 (d, J=8.3 Hz, 1H), 4.46-4.11 (m, 4H), 3.95-3.58 (m, 5H), 3.44 (d, J=16.3 Hz, 3H), 3.02 (d, J=7.5 Hz, 3H), 2.73-2.59 (m, 2H), 1.23-1.15 (m, 12H); ³¹P NMR (162 MHz, CD₃CN) δ=150.30, 150.10

### Example 4: Synthesis of 5′ End Cap Monomer

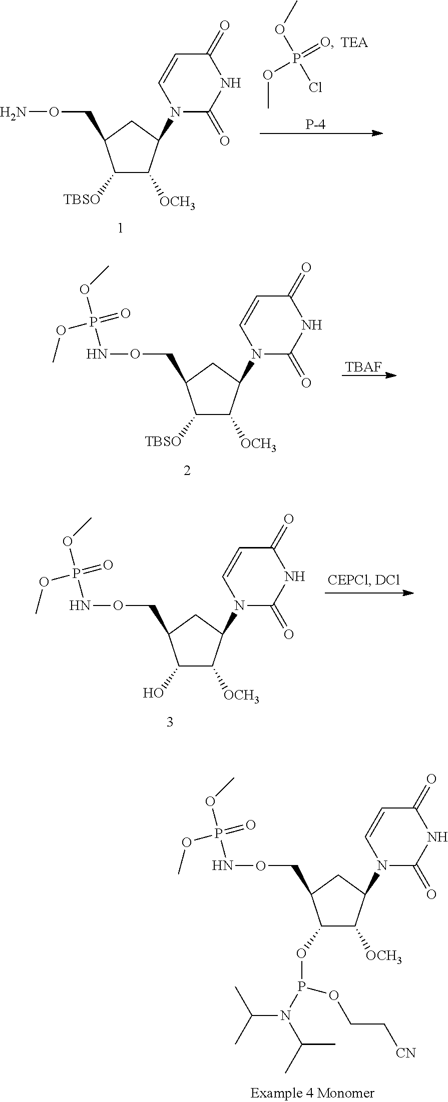

### Example 4 Monomer Synthesis Scheme

Preparation of (2): To the solution of 1 (5 g, 12.90 mmol) and TEA (1.57 g, 15.48 mmol, 2.16 mL) in DCM (50 mL) was added P-4 (2.24 g, 15.48 mmol, 1.67 mL) in DCM (10 mL) dropwise at 15° C. under N₂. The reaction mixture was stirred at 15° C. for 3 h. Upon completion as monitored by LCMS and TLC (PE:EtOAc=0:1), the reaction mixture was concentrated to dryness, diluted with H₂O (20 mL), and extracted with EA (50 mL*3). The combined organic layers were washed with brine (30 mL*3), dried over anhydrous Na₂SO₄, filtered, and the filtrate was concentrated under reduced pressure to give a residue. The residue was purified by flash silica gel chromatography (ISCO®; 40 g SepaFlash® Silica Flash Column, Eluent of 0˜95% Ethyl acetate/Petroleum ether gradient @60 mL/min) to give 2 (5.3 g, 71.3% yield) as a white solid. ESI-LCMS: 496.1 [M+H]⁺; H NMR (400 MHz, CDCl₃) δ=0.10 (d, J=4.02 Hz, 6H) 0.91 (s, 9H) 3.42-3.54 (m, 3H) 3.65-3.70 (m, 1H) 3.76-3.89 (m, 6H) 4.00 (dd, J=10.92, 2.89 Hz, 1H) 4.08-4.13 (m, 1H) 4.15-4.23 (m, 2H) 5.73 (dd, J=8.28, 2.01 Hz, 1H) 5.84 (d, J=2.76 Hz, 1H) 6.86 (d, J=15.81 Hz, 1H) 7.72 (d, J=8.03 Hz, 1H) 9.10 (s, 1H); ³¹P NMR (162 MHz, CD₃CN) δ=9.65

Preparation of (3): To a solution of 2 (8.3 g, 16.75 mmol) in THF (50 mL) were added TBAF (1 M, 16.75 mL) and CH₃COOH (1.01 g, 16.75 mmol, 957.95 uL). The mixture was stirred at 20° C. for 12 hr. Upon completion as monitored by LCMS, the reaction mixture was concentrated under reduced pressure. The residue was purified by column chromatography (SiO₂, PE:EA=0˜100%; MeOH/EA=0˜10%) to give 3 (5 g, 77.51% yield) as a white solid. ESI-LCMS: 382.1 [M+H]⁺; ¹H NMR (400 MHz, CDCl₃) δ=3.35 (s, 3H) 3.65 (br d, J=2.76 Hz, 3H) 3.68 (d, J=2.76 Hz, 3H) 3.77 (t, J=5.08 Hz, 1H) 3.84-4.10 (m, 4H) 5.33 (br d, J=5.52 Hz, 1H) 5.62 (d, J=7.77 Hz, 1H) 5.83 (d, J=4.94 Hz, 1H) 7.69 (d, J=7.71 Hz, 1H) 9.08 (d, J=16.81 Hz, 1H) 11.39 (br s, 1H); ³¹P NMR (162 MHz, CD₃CN) δ=15.41

Preparation of (Example 4 monomer): To a solution of 3 (2 g, 5.25 mmol) and DIPEA (2.03 g, 15.74 mmol, 2.74 mL, 3 eq) in MeCN (21 mL) and pyridine (7 mL) was added CEOP[N(iPr)₂]₂/CEP[N(iPr)₂]₂/CEP/CEPCl (1.86 g, 7.87 mmol) dropwise at 20° C., and the mixture was stirred at 20° C. for 3 hr. Upon completion as monitored by LCMS, the reaction mixture was diluted with water (20 mL) and extracted with EA (50 mL). The combined organic layers were washed with brine (30 mL), dried over anhydrous Na₂SO₄, filtered, and the filtrate was concentrated under reduced pressure to give a residue. The residue was purified by flash silica gel chromatography (ISCO®; 25 g SepaFlash® Silica Flash Column, Eluent of 0˜45% (Ethyl acetate:EtOH=4:1)/Petroleum ether gradient) to give Example 4 monomer (1.2 g, 38.2% yield) as a white solid. ESI-LCMS: 604.1 [M+H]⁺; H NMR (400 MHz, CD₃CN) δ=1.12-1.24 (m, 12H) 2.61-2.77 (m, 2H) 3.43 (d, J=17.64 Hz, 3H) 3.59-3.69 (m, 2H) 3.71-3.78 (m, 6H) 3.79-4.14 (m, 5H) 4.16-4.28 (m, 1H) 4.29-4.42 (m, 1H) 5.59-5.72 (m, 1H) 5.89 (t, J=4.53 Hz, 1H) 7.48 (br d, J=12.76 Hz, 1H) 7.62-7.74 (m, 1H) 9.26 (br s, 1H); ³¹P NMR (162 MHz, CD₃CN) δ=150.57, 149.96, 9.87

### Example 5: Synthesis of 5′ End Cap Monomer

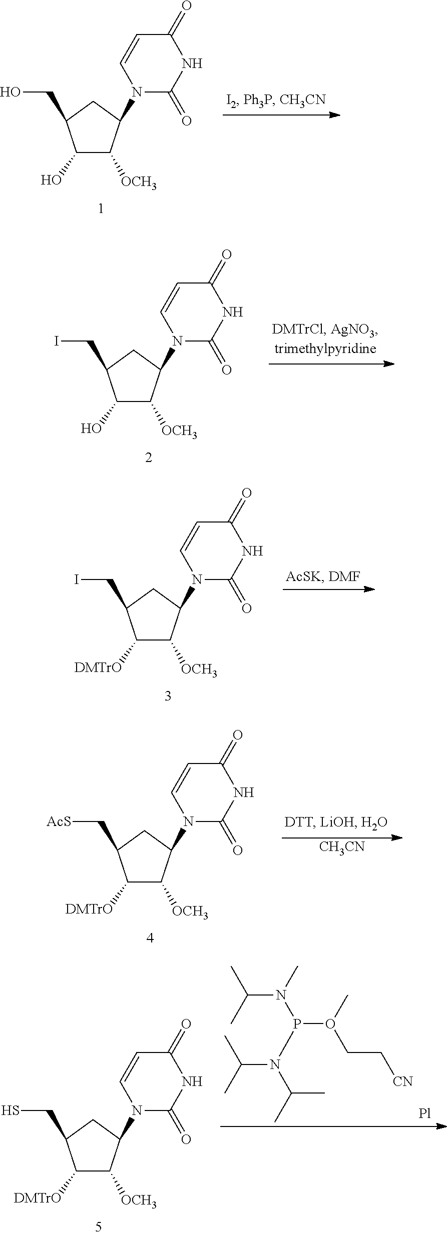

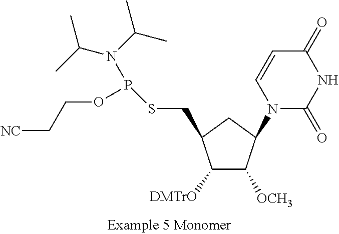

### Example 5 Monomer Synthesis Scheme

Preparation of (2): To a solution of 1 (30 g, 101.07 mmol, 87 purity) in CH₃CN (1.2 L) and Py (60 mL) were added 12 (33.35 g, 131.40 mmol, 26.47 mL) and PPh₃ (37.11 g, 141.50 mmol) in one portion at 10° C. The reaction was stirred at 25° C. for 48 h. Upon completion, the mixture was diluted with saturated aq·Na₂S₂O₃ (300 mL) and saturated aq·NaHCO₃ (300 mL), concentrated to remove CH₃CN, and extracted with EtOAc (300 mL*3). The combined organic layers were washed with brine (300 mL), dried over Na₂S04, filtered and concentrated under reduced pressure to give a residue. The residue was purified by flash silica gel chromatography (ISCO®; 330 g SepaFlash® Silica Flash Column, Eluent of 0-60% Methanol/Dichloromethane gradient @100 mL/min) to give 2 (28.2 g, 72% yield) as a brown solid. ESI-LCMS: 369.1 [M+H]⁺: H NM/R (400 MHz, DMSO-d₆) δ=11.43 (s, 1H), 7.68 (d, J=8.1 Hz, 1H), 5.86 (d, J=5.5 Hz, 1H), 5.69 (d, J=8.1 Hz, 1H), 5.46 (d, J=6.0 Hz, 1H), 4.08-3.96 (m, 2H), 3.90-3.81 (m, 1H), 3.60-3.51 (m, 1H), 3.40 (dd, J=6.9, 10.6 Hz, 1H), 3.34 (s, 3H).

Preparation of (3): To the solution of 2 (12 g, 32.6 mmol) in DCM (150 mL) were added AgNO₃ (11.07 g, 65.20 mmol), 2,4,6-trimethylpyridine (11.85 g, 97.79 mmol, 12.92 mL), and DMTCl (22.09 g, 65.20 mmol) at 10° C., and the reaction mixture was stirred at 10° C. for 16 hr. Upon completion, the mixture was filtered and the filtrate was concentrated under reduced pressure. The residue was purified by flash silica gel chromatography (ISCO®; 120 g SepaFlash® Silica Flash Column, Eluent of 0˜50% Ethyl acetate/Petroleum ethergradient @60 mL/min) to give 3 (17 g, 70.78% yield) as a yellow solid. ESI-LCMS: 693.1 [M+Na]⁺¹; H NMR (400 MHz, DMSO-d₆) δ=11.46 (s, 1H), 7.60 (d, J=8.4 Hz, 1H), 7.49 (d, J=7.2 Hz, 2H), 7.40-7.30 (m, 6H), 7.29-7.23 (m, 1H), 6.93 (d, J=8.8 Hz, 4H), 5.97 (d, J=6.0 Hz, 1H), 5.69 (d, J=8.0 Hz, 1H), 4.05-4.02 (m, 1H), 3.75 (d, J=1.2 Hz, 6H), 3.57 (t, J=5.6 Hz, 1H), 3.27 (s, 4H), 3.06 (t, J=10.4 Hz, 1H), 2.98-2.89 (m, 1H).

Preparation of (4): To a solution of 3 (17 g, 25.35 mmol) in DMF (200 mL) was added AcSK (11.58 g, 101.42 mmol) at 25° C., and the reaction was stirred at 60° C. for 2 hr. The mixture was diluted with H₂O (600 mL) and extracted with EtOAc (300 mL*4). The combined organic layers were washed with brine (300 mL), dried over Na₂SO₄, filtered, and concentrated under reduced pressure to give 4 (15.6 g, crude) as a brown solid, which was used directly without further purification. ESI-LCMS: 641.3 [M+H]⁺.

Preparation of (5): To a solution of 4 (15.6 g, 25.21 mmol) in CH₃CN (200 mL) were added DTT (11.67 g, 75.64 mmol, 11.22 mL) and LiOH·H₂O (1.06 g, 25.21 mmol) at 10° C. under Ar. The reaction was stirred at 10° C. for 1 hr. The mixture was concentrated under reduced pressure to remove CH₃CN, and the residue was diluted with H₂O (400 mL) and extracted with EtOAc (200 mL*3). The combined organic layers were washed with brine (300 mL), dried over Na₂SO₄, filtered and concentrated under reduced pressure to give a residue. The residue was purified by flash silica gel chromatography (ISCO®; 220 g SepaFlash® Silica Flash Column, Eluent of 0˜60% Ethyl acetate/Petroleum ether gradient @100 mL/min) to give 5 (8.6 g, 56.78% yield) as a white solid. ESI-LCMS: 599.3 [M+Na]⁺; ¹H NMR (400 MHz, DMSO-d₆) δ=8.79 (s, 1H), 7.61 (d, J=8.0 Hz, 1H), 7.56-7.46 (m, 2H), 7.45-7.37 (m, 4H), 7.36-7.27 (m, 3H), 6.85 (dd, J=2.8, 8.8 Hz, 4H), 5.85 (d, J=1.3 Hz, 1H), 5.68 (dd, J=2.0, 8.2 Hz, 1H), 4.33-4.29 (m, 1H), 3.91 (dd, J=4.8, 8.2 Hz, 1H), 3.81 (d, J=1.6 Hz, 6H), 3.33 (s, 3H), 2.85-2.80 (m, 1H), 2.67-2.55 (m, 2H), 1.11 (t, J=8.8 Hz, 1H).

Preparation of (Example 5 monomer): To a solution of 5 (6 g, 10.40 mmol) in DCM (120 mL) were added P1 (4.08 g, 13.53 mmol, 4.30 mL) and DCI (1.35 g, 11.45 mmol) in one portion at 10° C. under Ar. The reaction was stirred at 10° C. for 2 hr. The reaction mixture was diluted with saturated aq·NaHCO₃ (50 mL) and extracted with DCM (20 mL*3). The combined organic layers were washed with brine (30 mL), dried over Na₂SO₄, filtered and concentrated under reduced pressure to give a residue. The residue was purified by prep-HPLC (column: YMC-Triart Prep C18 250*50 mm*10 um; mobile phase: [water (10 mM NH₄HCO₃)-ACN]; B %: 35%-81%, 20 min) to give Example 5 monomer (3.54 g, 43.36% yield) as a yellow solid. ESI-LCMS: 776.4 [M+H]⁺; ¹H NMR (400 MHz, DMSO-d₆) δ=7.65-7.38 (m, 7H), 7.37-7.22 (m, 3H), 6.90 (d, J=8.4 Hz, 4H), 5.92 (s, 1H), 5.66 (t, J=8.2 Hz, 1H), 4.13 (d, J=4.0 Hz, 1H), 4.00-3.88 (m, 1H), 3.87-3.59 (m, 10H), 3.33 (d, J=5.8 Hz, 3H), 3.12-2.94 (m, 1H), 2.78-2.60 (m, 3H), 2.55-2.48 (m, 1H), 1.36-0.98 (m, 12H); ³¹P NMR (162 MHz, DMSO-d₆) δ=162.69.

### Example 6: Synthesis of 5′ End Cap Monomer

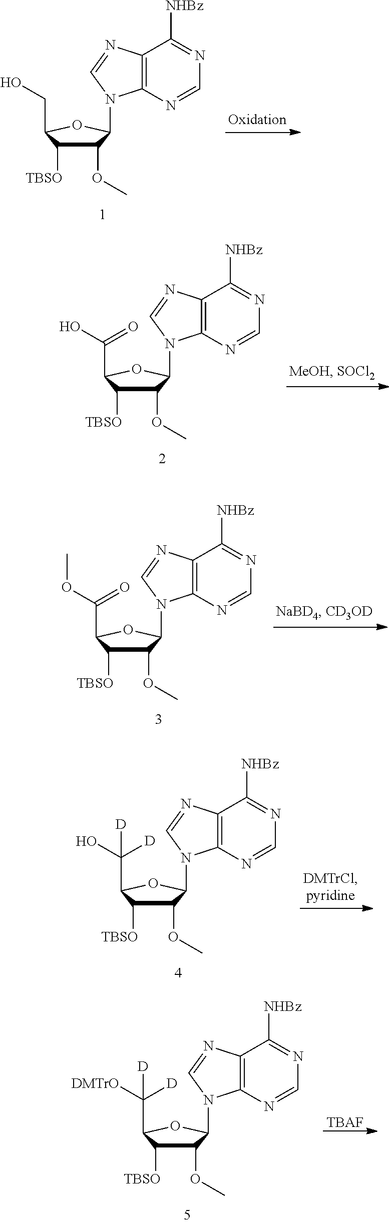

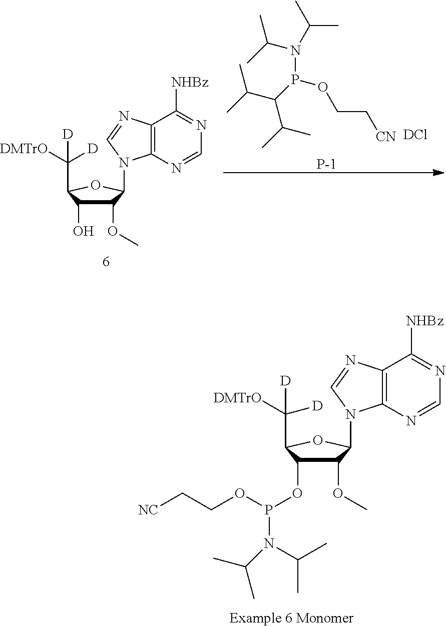

### Example 6 Monomer Synthesis Scheme

Preparation of (2): To a solution of 1 (22.6 g, 45.23 mmol) in DCM (500 mL) and H₂O (125 mL) were added TEMPO (6.40 g, 40.71 mmol) and DIB (29.14 g, 90.47 mmol) at 0° C. The mixture was stirred at 20° C. for 20 h. Upon completion as monitored by LCMS, saturated aq·NaHCO₃ was added to the mixture to adjust pH >8. The mixture was diluted with H₂O (200 mL) and washed with DCM (100 mL*3). The aqueous layer was collected, adjusted to pH<5 by HCl (4M), and extracted with DCM (200 mL*3). The combined organic layers were washed with brine (300 mL), dried over Na₂SO₄, filtered, and concentrated under reduced pressure to give 2 (17.5 g, 68.55% yield) as a yellow solid. ESI-LCMS: 514.2 [M+H]⁺; ¹H NMR (400 MHz, DMSO-d₆) δ=11.27 (s, 1H), 8.86 (s, 1H), 8.78 (s, 1H), 8.06 (d, J=7.5 Hz, 2H), 7.68-7.62 (m, 1H), 7.59-7.52 (m, 2H), 6.28 (d, J=6.8 Hz, 1H), 4.82-4.76 (m, 1H), 4.54 (dd, J=4.1, 6.7 Hz, 1H), 4.48 (d, J=1.8 Hz, 1H), 3.32 (s, 3H), 0.94 (s, 9H), 0.18 (d, J=4.8 Hz, 6H).

Preparation of (3): To a solution of 2 (9.3 g, 18.11 mmol) in MeOH (20 mL) was added SOCl₂ (3.23 g, 27.16 mmol, 1.97 mL) dropwise at 0° C. The mixture was stirred at 20° C. for 0.5 hr. Upon completion as monitored by LCMS, the reaction mixture was quenched by addition of saturated aq·NaHCO₃ (80 mL) and concentrated under reduced pressure to remove MeOH. The aqueous layer was extracted with DCM (80 mL*3). The combined organic layers were washed with brine (200 mL), dried over Na₂SO₄, filtered and concentrated under reduced pressure to give a residue. The residue was purified by flash silica gel chromatography (ISCO®; 120 g SepaFlash® Silica Flash Column, Eluent of 0˜5%, MeOH/DCM gradient @85 mL/min) to give 3 (5.8 g, 60% yield) as a yellow solid. ESI-LCMS: 528.3 [M+H]⁺; ¹H NMR (400 MHz, DMSO-d₆) δ=11.28 (s, 1H), 8.79 (d, J=7.3 Hz, 2H), 8.06 (d, J=7.5 Hz, 2H), 7.68-7.62 (m, 1H), 7.60-7.53 (m, 2H), 6.28 (d, J=6.6 Hz, 1H), 4.87 (dd, J=2.4, 4.0 Hz, 1H), 4.61 (dd, J=4.3, 6.5 Hz, 1H), 4.57 (d, J=2.2 Hz, 1H), 3.75 (s, 3H), 3.32 (s, 3H), 0.94 (s, 9H), 0.17 (d, J=2.2 Hz, 6H).

Preparation of (4): To a mixture of 3 (5.7 g, 10.80 mmol) in CD₃OD (120 mL) was added NaBD₄ (1.63 g, 43.21 mmol) in portions at 0° C., and the mixture was stirred at 20° C. for 1 hr. Upon completion as monitored by LCMS, the reaction mixture was neutralized by AcOH (˜10 mL) and concentrated under reduced pressure to give a residue. The residue was purified by flash silica gel chromatography (ISCO®; 40 g SepaFlash® Silica Flash Column, Eluent of 0˜5%, MeOH/DCM gradient @40 mL/min) to give 4 (4.15 g, 7.61 mmol, 70.45% yield) as a yellow solid. ESI-LCMS: 502.2 [M+H]⁺; ¹H NMR (400 MHz, DMSO-d₆) δ=11.23 (s, 1H), 8.76 (s, 2H), 8.04 (d, J=7.3 Hz, 2H), 7.69-7.62 (m, 1H), 7.60-7.52 (m, 2H), 6.14 (d, J=6.0 Hz, 1H), 5.18 (s, 1H), 4.60-4.51 (m, 2H), 3.98 (d, J=3.0 Hz, 1H), 3.32 (s, 3H), 0.92 (s, 9H), 0.13 (d, J=1.5 Hz, 6H).

Preparation of (5): To a solution of 4 (4.85 g, 9.67 mmol) in pyridine (50 mL) was added DMTrCl (5.90 g, 17.40 mmol) at 25° C. and the mixture was stirred for 2 hr. Upon completion as monitored by LCMS, the reaction mixture was concentrated under reduced pressure to remove pyridine. The residue was diluted with EtOAc (150 mL) and washed with H₂O (50 mL*3), dried over Na₂SO₄, filtered and concentrated under reduced pressure to give a residue. The residue was purified by flash silica gel chromatography (ISCO®; 80 g SepaFlash® Silica Flash Column, Eluent of 0˜70%, EA/PE gradient @60 mL/min) to give 5 (6.6 g, 84.06% yield) as a yellow solid. ESI-LCMS: 804.3[M+H]⁺, ¹H NMR (400 MHz, DMSO-d₆) δ=11.22 (s, 1H), 8.68 (d, J=11.0 Hz, 2H), 8.03 (d, J=7.3 Hz, 2H), 7.68-7.60 (m, 1H), 7.58-7.49 (m, 2H), 7.37-7.30 (m, 2H), 7.27-7.16 (m, 7H), 6.88-6.79 (m, 4H), 6.17 (d, J=4.2 Hz, 1H), 4.72 (t, J=5.0 Hz, 1H), 4.60 (t, J=4.5 Hz, 1H), 4.03-3.98 (m, 1H), 3.71 (s, 6H), 0.83 (s, 9H), 0.12-0.03 (m, 6H).

Preparation of (6): To a solution of 5 (6.6 g, 8.21 mmol) in THF (16 mL) was added TBAF (1 M, 8.21 mL), and the mixture was stirred at 20° C. for 2 hr. Upon completion as monitored by LCMS, the reaction mixture was diluted with EA (150 mL) and washed with H₂O (50 mL*3). The organic layer was washed with brine (150 mL), dried over Na₂SO₄, filtered and concentrated under reduced pressure to give a residue. The residue was purified by flash silica gel chromatography (ISCO®; 80 g SepaFlash® Silica Flash Column, Eluent of 10-100%, EA/PE gradient @30 mL/min) to give 6 (5.4 g, 94.4% yield) as a yellow solid. ESI-LCMS: 690.3 [M+H]⁺; ¹H NMR (400 MHz, DMSO-d₆) δ=11.24 (s, 1H), 8.69 (s, 1H), 8.62 (s, 1H), 8.05 (d, J=7.3 Hz, 2H), 7.69-7.62 (m, 1H), 7.60-7.52 (m, 2H), 7.40-7.33 (m, 2H), 7.30-7.18 (m, 7H), 6.84 (dd, J=5.9, 8.9 Hz, 4H), 6.19 (d, J=4.8 Hz, 1H), 5.36 (d, J=6.0 Hz, 1H), 4.59-4.52 (m, 1H), 4.48 (q, J=5.1 Hz, 1H), 4.11 (d, J=4.8 Hz, 1H), 3.72 (d, J=1.0 Hz, 6H), 3.40 (s, 3H).

Preparation of (Example 6 monomer): To a solution of 6 (8.0 g, 11.60 mmol) in MeCN (150 mL) was added P-1 (4.54 g, 15.08 mmol, 4.79 mL) at 0° C., followed by DCI (1.51 g, 12.76 mmol) in one portion. The mixture was warmed to 20° C. and stirred for 2 h. Upon completion as monitored by LCMS, the reaction mixture was quenched by addition of saturated aq·NaHCO₃ (50 mL) and diluted with DCM (250 mL). The organic layer was washed with saturated aq·NaHCO₃ (50 mL*2), dried over Na₂SO₄, filtered and concentrated under reduced pressure. The residue was purified by a flash silica gel column (0% to 60% EA in PE contain 0.5% TEA) to give Example 6 monomer (5.75 g, 55.37% yield, 99.4% purity) as a white solid. ESI-LCMS: 890.4 [M+H]⁺; ¹H NMR (400 MHz, CD₃CN) δ=9.55 (s, 1H), 8.63-8.51 (m, 1H), 8.34-8.24 (m, 1H), 7.98 (br d, J=7.5 Hz, 2H), 7.65-7.55 (m, 1H), 7.53-7.46 (m, 2H), 7.44-7.37 (m, 2H), 7.32-7.17 (m, 7H), 6.84-6.77 (m, 4H), 6.14 (d, J=4.3 Hz, 1H), 4.84-4.73 (m, 1H), 4.72-4.65 (m, 1H), 4.34-4.27 (m, 1H), 3.91-3.61 (m, 9H), 3.50-3.43 (m, 3H), 2.72-2.61 (m, 1H), 2.50 (t, J=6.0 Hz, 1H), 1.21-1.15 (m, 10H), 1.09 (d, J=6.8 Hz, 2H); ³¹P NMR (162 MHz, CD₃CN) δ=150.01, 149.65

### Example 7: Synthesis of 5′ End Cap Monomer

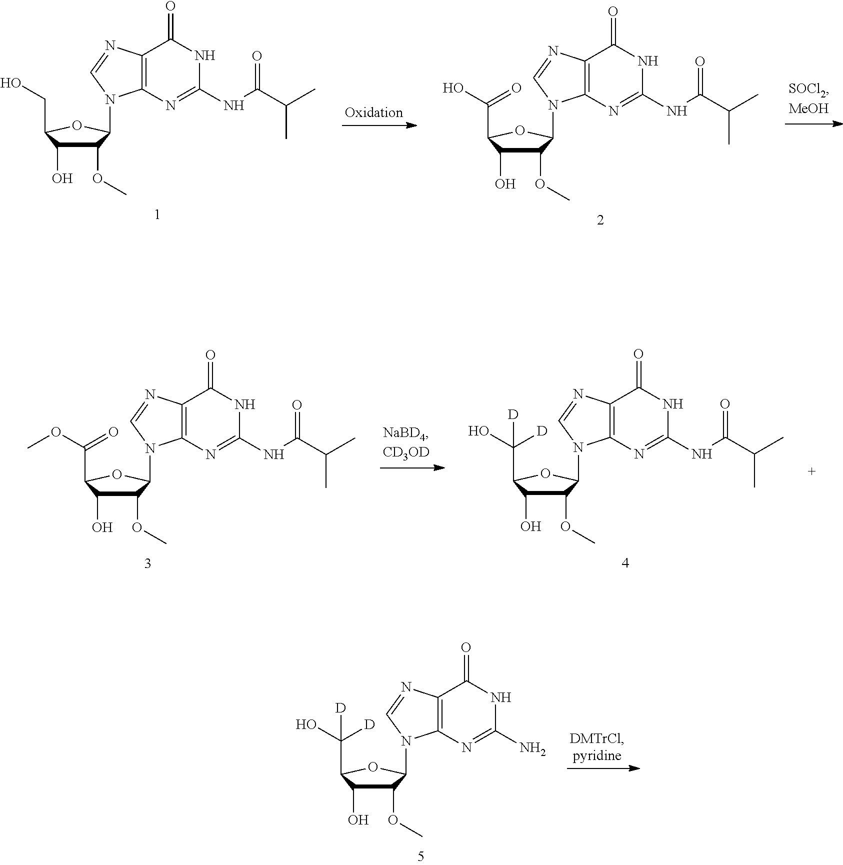

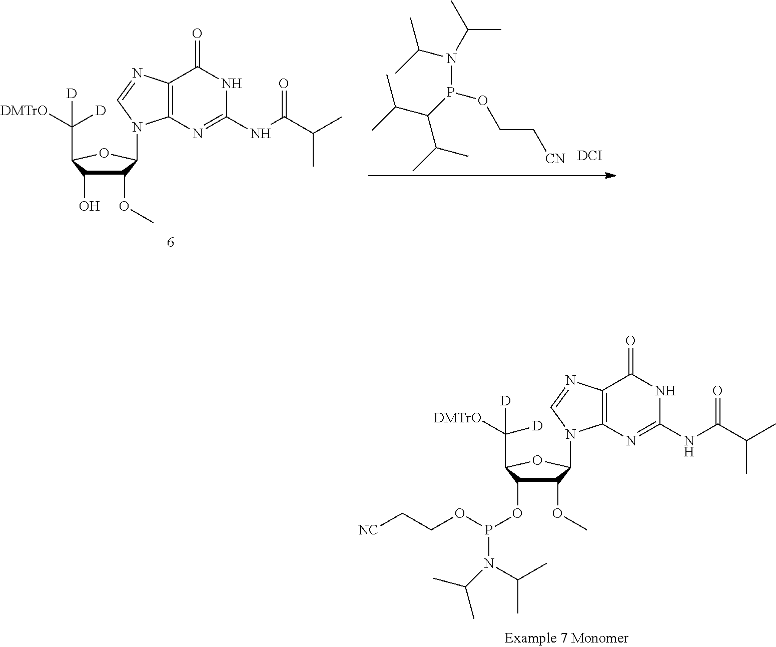

### Example 7 Monomer Synthesis Scheme

Preparation of (2): To a solution of 1 (10 g, 27.22 mmol) in CH₃CN (200 mL) and H₂O (50 mL) were added TEMPO (3.85 g, 24.50 mmol) and DIB (17.54 g, 54.44 mmol). The mixture was stirred at 25° C. for 12 h. Upon completion as monitored by LCMS, the reaction mixture was concentrated under reduced pressure to give a residue. The residue was triturated with EtOAc (600 mL) for 30 min. The resulting suspension was filtered and the collected solid was washed with EtOAc (300 mL*2) to give 2 (20.09 g, 91.5% yield) as a white solid. ESI-LCMS: 382.0 [M+H]⁺.

Preparation of (3): To a solution of 2 (6 g, 15.73 mmol) in MeOH (100 mL) was added SOCl₂ (2.81 g, 23.60 mmol, 1.71 mL) dropwise at 0° C. The mixture was stirred at 25° C. for 12 h. Upon completion as monitored by LCMS, the reaction mixture was quenched by addition of NaHCO₃ (4 g) and stirred at 25° C. for 30 min. The reaction mixture was filtered and the filtrate was concentrated under reduced pressure to give 3 (18.8 g, 95.6% yield) as a white solid. The crude product was used for the next step without further purification. (The reaction was set up in parallel 3 batches and combined). ESI-LCMS: 396.1 [M+H]⁺; ¹H NMR (400 MHz, DMSO-d₆) δ=12.26-11.57 (m, 2H), 8.42-8.06 (m, 1H), 6.14-5.68 (m, 2H), 4.56 (s, 2H), 4.33 (dd, J=4.0, 7.3 Hz, 1H), 3.77 (m, 3H), 3.30 (s, 3H), 2.81-2.69 (m, 1H), 1.11 (s, 6H)

Preparation of (4 & 5): To a mixture of 3 (10.1 g, 25.55 mmol) in CD₃OD (120 mL) was added NaBD₄ (3.29 g, 86.86 mmol, 3.4 eq) in portions at 0° C. The mixture was stirred at 25° C. for 1 h. Upon completion as monitored by LCMS, the reaction mixture was neutralized with AcOH (˜15 mL) and concentrated under reduced pressure to give a residue. The residue was purified by flash silica gel chromatography (ISCO®; 120 g SepaFlash® Silica Flash Column, Eluent of 0˜7.4%, MeOH/DCM gradient @80 mL/min) to give 4 (2.98 g, 6.88 mmol, 27% yield) as a yellow solid. ESI-LCMS: 370.1[M+H]⁺ and 5 (10.9 g, crude) as a yellow solid. ESI-LCMS: 300.1[M+H]⁺; ¹H NMR (400 MHz, CD₃OD) δ=7.85 (s, 1H), 5.87 (d, J=6.0 Hz, 1H), 4.46-4.39 (m, 1H), 4.34 (t, J=5.4 Hz, 1H), 4.08 (d, J=3.1 Hz, 1H), 3.49-3.38 (m, 4H)

Preparation of 6: To a solution of 4 (1.9 g, 4.58 mmol, 85.7% purity) in pyridine (19 mL) was added DMTrCl (2.02 g, 5.96 mmol). The mixture was stirred at 25° C. for 2 h under N₂. Upon completion as monitored by LCMS, the reaction mixture was quenched by MeOH (10 mL) and concentrated under reduce pressure to give a residue. The residue was diluted with H₂O (10 mL*3) and extracted with EA (20 mL*3). The combined organic layers were washed with brine (20 mL), dried over anhydrous Na₂SO₄, filtered and concentrated under reduce pressure to give a residue. The residue was purified by flash silica gel chromatography (ISCO®; 25 g SepaFlash® Silica Flash Column, Eluent of 0˜77%, PE:(EA with 10% EtOH): 1% TEA@35 mL/min) to give 6 (2.6 g, 81.71% yield, 96.71% purity) as a white foam. ESI-LCMS: 672.2 [M+H]⁺; ¹H NMR (400 MHz, CDCl₃) δ=12.02 (s, 1H), 7.96 (s, 1H), 7.83 (s, 1H), 7.51 (d, J=7.4 Hz, 2H), 7.37 (d, J=8.6 Hz, 4H), 7.25-7.17 (m, 2H), 6.80 (t, J=8.4 Hz, 4H), 5.88 (d, J=6.3 Hz, 1H), 4.69 (t, J=5.7 Hz, 1H), 4.64 (s, 1H), 4.54 (s, 1H), 4.19 (d, J=2.9 Hz, 1H), 3.77 (d, J=4.5 Hz, 6H), 3.60-3.38 (m, 3H), 2.81 (s, 1H), 1.81 (td, J=6.9, 13.7 Hz, 1H), 0.97 (d, J=6.8 Hz, 3H), 0.80 (d, J=6.9 Hz, 3H).

Preparation of Example 7 monomer: To a solution of 6 (8.4 g, 12.5 mmol) in MeCN (80 mL) was added P-1 (4.9 g, 16.26 mmol, 5.16 mL) at 0° C., followed by addition of DCI (1.624 g, 13.76 mmol) in one portion at 0° C. under Ar. The mixture was stirred at 25° C. for 2 h. Upon completion as monitored by LCMS, the reaction mixture was quenched with saturated aq·NaHCO₃ (20 mL) and extracted with DCM (50 mL*2). The combined organic layers were dried over anhydrous Na₂SO₄, filtered and concentrated under reduce pressure to give a residue. The residue was purified by flash silica gel chromatography (ISCO®; 40 g SepaFlash® Silica Flash Column, Eluent of 0˜52% PE:EA (10% EtOH): 5% TEA, @80 mL/min) to give Example 7 monomer (3.4 g, 72.1% yield) as a white foam. ESI-LCMS: 872.4 [M+H]⁺; ¹H NMR (400 MHz, CD₃CN) δ=12.46-11.07 (m, 1H), 9.29 (s, 1H), 7.84 (d, J=14.6 Hz, 1H), 7.42 (t, J=6.9 Hz, 2H), 7.34-7.17 (m, 7H), 6.85-6.77 (m, 4H), 5.95-5.77 (m, 1H), 4.56-4.40 (m, 2H), 4.24 (dd, J=4.0, 13.3 Hz, 1H), 3.72 (d, J=2.0 Hz, 7H), 3.66-3.53 (m, 3H), 3.42 (d, J=11.8 Hz, 3H), 2.69-2.61 (m, 1H), 2.60-2.42 (m, 2H), 1.16-1.00 (m, 18H); ³¹P NMR (162 MHz, CD₃CN) δ=149.975, 149.9.

### Example 8: Synthesis of 5′ End Cap Monomer

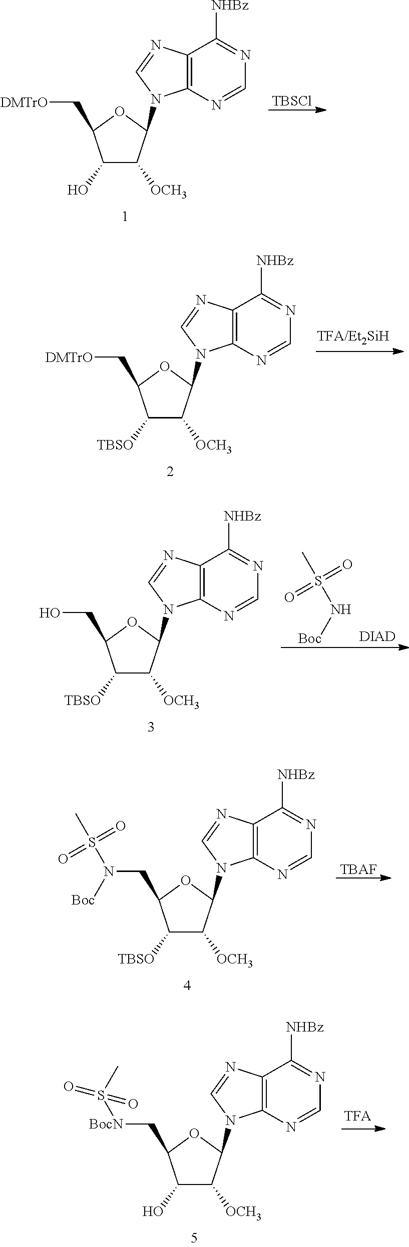

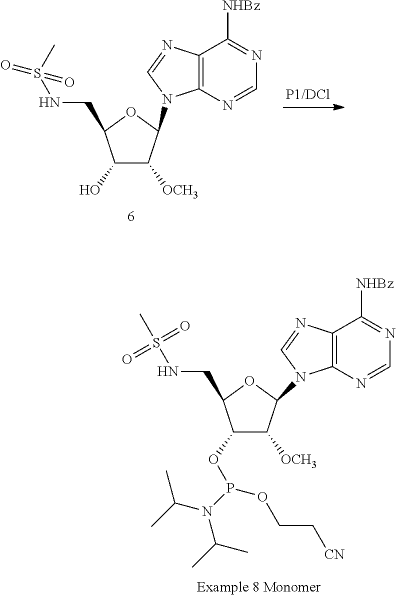

### Example 8 Monomer Synthesis Scheme

Preparation of (2): To a solution of 1 (40 g, 58.16 mmol) in DMF (60 mL) were added imidazole (11.88 g, 174.48 mmol), NaI (13.08 g, 87.24 mmol), and TBSCl (17.52 g, 116.32 mmol) at 20° C. in one portion. The reaction mixture was stirred at 20° C. for 12 h. Upon completion, the mixture was diluted with EA (200 mL). The organic layer was washed with brine/water (80 mL/80 mL*4), dried over Na₂SO₄, filtered and evaporated to give 2 (50.8 g, crude) as yellow solid. ESI-LCMS: 802.3 [M+H]⁺

Preparation of (3): To a solution of 2 (8.4 g, 10.47 mmol) in DCM (120 mL) were added Et₃S¹H (3.06 g, 26.3 mmol, 4.2 mL) and TFA (1.29 g, 0.84 mL) dropwise at 0° C. The reaction mixture was stirred at 20° C. for 2 h. The reaction mixture was washed with saturated aq·NaHCO₃ (15 mL) and brine (80 mL). The organic layer was dried over Na₂SO₄, filtered and evaporated. The residue was purified by flash silica gel chromatography (ISCO@; 80 g SepaFlash® Silica Flash Column, Eluent of 0˜83% EA/PE gradient @80 mL/min) to give 3 (2.92 g, 55.8% yield,) as a white solid. ESI-LCMS: 500.2 [M+H]⁺; ¹H NMR (400 MHz, CDCl₃) δ=8.79 (s, 1H), 8.14 (s, 1H), 8.02 (d, J=7.6 Hz, 2H), 7.64-7.58 (m, 1H), 7.56-7.49 (m, 2H), 5.98-5.93 (m, 1H), 4.63-4.56 (m, 2H), 4.23 (s, 1H), 3.98 (dd, J=1.5, 13.1 Hz, 1H), 3.75 (dd, J=1.5, 13.1 Hz, 1H), 3.28 (s, 3H), 2.06-1.99 (m, 1H), 1.00-0.90 (m, 9H), 0.15 (d, J=7.0 Hz, 6H).

Preparation of (4): 3 (6 g, 12.01 mmol) and tert-butyl N-methylsulfonylcarbamate (3.52 g, 18.01 mmol) were co-evaporated with toluene (50 mL), dissolved in dry THF (100 mL), and cooled to 0° C. PPh₃ (9.45 g, 36.03 mmol,) was then added, followed by dropwise addition of DIAD (7.28 g, 36.03 mmol, 7.00 mL) in dry THF (30 mL). The reaction mixture was stirred at 20° C. for 18 h. Upon completion, the reaction mixture was then diluted with DCM (100 mL) and washed with water (70 mL) and brine (70 mL), dried over Na₂SO₄, filtered and evaporated to give a residue. The residue was purified by flash silica gel chromatography (ISCO®; 80 g SepaFlash® Silica Flash Column, Eluent of 0˜100% Ethyl acetate/Petroleum ether gradient @60 mL/min) followed by reverse-phase HPLC (0.1% NH₃·H₂O condition, eluent at 74%) to give 4 (2.88 g, 25% yield) as a white solid. ESI-LCMS: 677.1 [M+H]⁺; ¹H NMR (400 MHz, CDCl₃) δ=9.24 (s, 1H), 8.84 (s, 1H), 8.36 (s, 1H), 8.05 (br d, J=7.3 Hz, 2H), 7.66-7.42 (m, 4H), 6.16 (d, J=5.0 Hz, 1H), 4.52 (br t, J=4.5 Hz, 1H), 4.25-4.10 (m, 1H), 3.97 (br dd, J=8.0, 14.8 Hz, 1H), 3.48 (s, 3H), 3.27 (s, 3H), 1.54 (s, 9H), 0.95 (s, 9H), 0.14 (d, J=0.8 Hz, 6H).

Preparation of (5): To a solution of 4 (2.8 g, 4.14 mmol) in THF (20 mL) was added TBAF (4 M, 1.03 mL) and the mixture was stirred at 20° C. for 12 h. The reaction mixture was then evaporated. The residue was purified by flash silica gel chromatography (ISCO®; 12 g SepaFlash® Silica Flash Column, Eluent of 0˜6% MeOH/ethyl acetate gradient @20 mL/min) to give 5 (2.1 g, 83.92% yield) as a white solid. ESI-LCMS: 563.1[M+H]⁺; ¹H NMR (400 MHz, CDCl₃) δ=8.85-8.77 (m, 1H), 8.38 (s, 1H), 8.11-7.99 (m, 2H), 7.64-7.50 (m, 4H), 6.19 (d, J=2.8 Hz, 1H), 4.36-4.33 (m, 1H), 4.29 (br d, J=4.3 Hz, 1H), 4.22-4.02 (m, 2H), 3.65-3.59 (m, 3H), 3.28 (s, 3H), 1.54 (s, 9H).

Preparation of (6): To a solution of 5 (2.1 g, 3.73 mmol) in DCM (20 mL) was added TFA (7.70 g, 67.53 mmol, 5 mL) at 0° C. The reaction mixture was stirred at 20° C. for 24 h. Upon completion, the reaction was quenched with saturated aq·NaHCO₃ to reach pH 7. The organic layer was dried over Na₂SO₄, filtered, and evaporated at low pressure. The residue was purified by flash silica gel chromatography (ISCO@; 12 g SepaFlash® Silica Flash Column, Eluent of 0˜7% DCM/MeOH gradient @20 mL/min) to give 1.6 g (impure, 75% LCMS purity), followed by prep-HPLC [FA condition, column: Boston Uni C18 40*150*5 um; mobile phase: [water (0.225% FA)-ACN]; B %: 8%-38%, 7.7 min.] to give 6 (1.04 g, 63.7% yield) as a white solid. ESI-LCMS: 485.0 [M+Na]⁺; ¹H NMR (400 MHz, DMSO-d₆) δ=11.27-11.21 (m, 1H), 8.77 (s, 1H), 8.74 (s, 1H), 8.05 (d, J=7.3 Hz, 2H), 7.68-7.62 (m, 1H), 7.59-7.53 (m, 2H), 7.39 (t, J=6.3 Hz, 1H), 6.16 (d, J=6.0 Hz, 1H), 5.48 (d, J=5.5 Hz, 1H), 4.55 (t, J=5.5 Hz, 1H), 4.43-4.37 (m, 1H), 4.08-4.02 (m, 1H), 3.41-3.36 (m, 1H), 3.35 (s, 3H), 3.31-3.22 (m, 1H), 2.91 (s, 3H).

Preparation of (Example 8 monomer): To a solution of 6 (1 g, 2.16 mmol) in DCM (30 mL) was added P1 (977.58 mg, 3.24 mmol, 1.03 mL), followed by DCI (306.43 mg, 2.59 mmol) at 0° C. in one portion under Ar atmosphere. The mixture was degassed and purged with Ar for 3 times, warmed to 20° C., and stirred for 2 hr under Ar atmosphere. Upon completion as monitored by LCMS and TLC (PE:EtOAc=4:1), the reaction mixture was diluted with sat·aq·NaHCO₃ (30 mL) and extracted with DCM (50 mL*2). The combined organic layers were dried over anhydrous Na₂SO₄, filtered, and the filtrate was concentrated under reduced pressure to give a residue. The crude product was purified by reversed-phase HPLC (40 g C18 column: neutral condition, Eluent of 0˜57% of 0.3% NH₄HCO₃ in H₂O/CH₃CN ether gradient @35 mL/min) to give Example 8 monomer (0.49 g, 33.7% yield) as a white solid. ESI-LCMS: 663.1[M+H]⁺; ¹H NMR (400 MHz, CD₃CN) δ=1.19-1.29 (m, 12H) 2.71 (q, J=5.77 Hz, 2H) 2.94 (d, J=6.27 Hz, 3H) 3.35 (d, J=15.56 Hz, 3H) 3.40-3.52 (m, 2H) 3.61-3.97 (m, 4H) 4.23-4.45 (m, 1H) 4.55-4.74 (m, 2H) 6.02 (dd, J=10.67, 6.40 Hz, 1H) 7.25 (br s, 1H) 7.47-7.57 (m, 2H) 7.59-7.68 (m, 1H) 8.01 (d, J=7.78 Hz, 2H) 8.28 (s, 1H) 8.66 (s, 1H) 9.69 (br s, 1H); ³¹P NMR (162 MHz, CD₃CN) δ=150.92, 149.78.

### Example 9. Synthesis of 5′-Stabilized End Cap Modified Oligonucleotides

This example provides an exemplary method for synthesizing the siNAs comprising a 5′-stabilized end caps disclosed herein. The 5′-stabilized end cap and/or deuterated phosphoramidites were dissolved in anhydrous acetonitrile and oligonucleotide synthesis was performed on a Expedite 8909 Synthesizer using standard phosphoramidite chemistry. An extended coupling (12 minutes) of 0.12 M solution of phosphoramidite in anhydrous CH₃CN in the presence of Benzyl-thio-tetrazole (BTT) activator to a solid bound oligonucleotide followed by standard capping, oxidation and sulfurization produced modified oligonucleotides. The 0.02 M 12, THF:Pyridine; Water 7:2:1 was used as an oxidizing agent, while DDTT (dimethylamino-methylidene)amino)-3H-1,2,4-dithiazaoline-3-thione was used as the sulfur-transfer agent for the synthesis of oligoribonucleotide with a phosphorothioate backbone. The stepwise coupling efficiency of all modified phosphoramidites was achieved around 98%. After synthesis the solid support was heated with aqueous ammonia (28%) solution at 45° C. for 16 h or 0.05 M K2CO3 in methanol was used to deprotect the base labile protecting groups. The crude oligonucleotides were precipitated with isopropanol and centrifuged (Eppendorf 5810R, 3000 g, 4° C., 15 min) to obtain a pellet. The crude product was then purified using ion exchange chromatography (TSK gel column, 20 mM NaH₂PO₄, 10% CH₃CN, 1 M NaBr, gradient 20-60% 1 M NaBr over 20 column volumes) and fractions were analyzed by ion change chromatography on an HPLC. Pure fractions were pooled and desalted by Sephadex G-25 column and evaporated to dryness. The purity and molecular weight were determined by HPLC analysis and ESI-MS analysis. Single strand RNA oligonucleotides (sense and antisense strand) were annealed (1:1 by molar equivalents) at 90° C. for 3 min followed by RT 40 min) to produce the duplexes.

### Example 10. Synthesis of Monomer

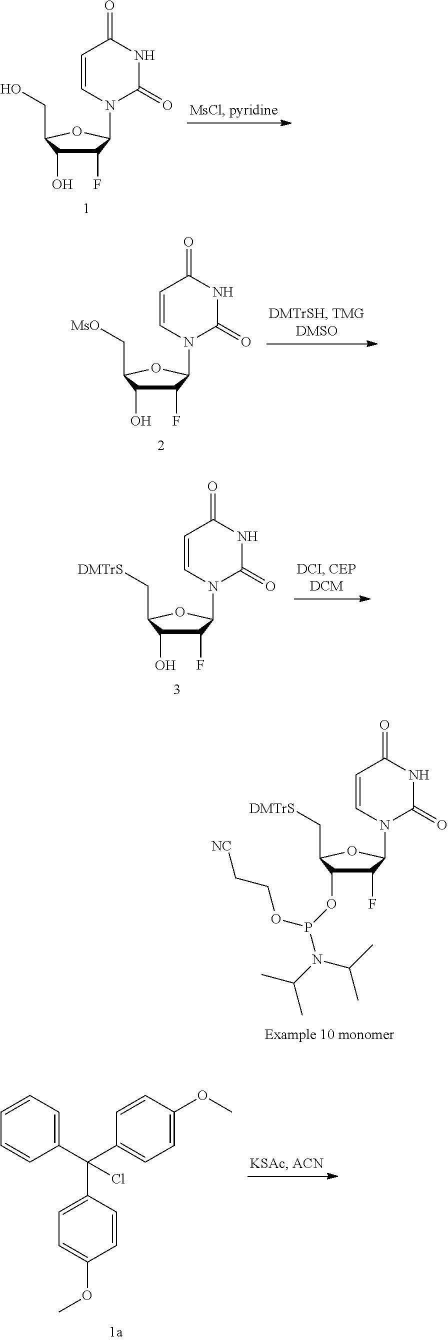

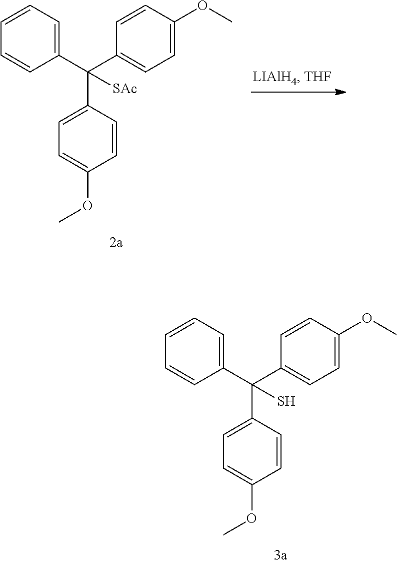

Preparation of (2a): To a solution of 1a (10.0 g, 29.5 mmol) in ACN (200.0 mL), KSAc (13.5 g, 118.6 mmol) was added at r.t., the mixture was stirred at r.t. for 15 h, TLC showed 1a was consumed completely. Mixture was filtered by silica gel and filter cake was washed with DCM (100.0 mL), the filtrate was concentrated to give crude 2a (11.1 g) as an oil. ¹H-NMR (400 MHz, CDCl₃): δ 7.32-7.24 (m, 5H), 7.16 (d, J=8.9 Hz, 4H), 6.82 (d, J=8.9 Hz, 4H), 3.82 (s, 6H), 2.28 (s, 3H).

Preparation of (3a): To a solution of crude 2a (11.1 g, 29.2 mmol) in THF (290.0 mL), LiAlH₄ (2.0 g, 52.6 mmol) was added at 0° C. and kept for 10 min, reaction was stirred at r.t. for 5 h under N₂, TLC showed 2a was consumed completely. Mixture was put into aqueous NaHCO₃ solution and extracted with EA (500.0 mL*2), organic phase was concentrated to give crude which was purified by column chromatography (SiO2, PE/EA=30:1 to 10:1) to give 3a (8.1 g, 95% purity) as a white solid. ESI-LCMS: m/z 335.3 [M−H]⁻; ¹H-NMR (400 MHz, CDCl₃): δ 7.33-7.24 (m, 5H), 7.19 (d, J=8.8 Hz, 4H), 6.82 (d, J=8.8 Hz, 4H), 3.83 (s, 6H), 3.09 (s, 1H).

Preparation of (2): To a solution of 1 (20.0 g, 81.3 mmol) in pyridine (400.0 mL), MsCl (10.23 g, 89.43 mmol) was added dropwise at −10° C., reaction was stirred at −10° C. for 1 h, LCMS showed 1 was consumed completely, 100.0 mL aqueous NaHCO₃ solution was added and extracted with DCM (100.0 mL*2), organic phase was concentrated to give crude which was purified by column chromatography (SiO2, DCM/MeOH=30:1 to 10:1) to give 2 (9.5 g, 97% purity) as a white solid. ESI-LCMS: m/z 325.3 [M+H]⁺; ¹H-NMR (400 MHz, DMSO-d₆): δ 11.45 (s, 1H), 7.64-7.62 (d, J=8.0 Hz, 1H), 5.92-5.85 (m, 2H), 5.65-5.63 (d, J=8.0 Hz, 1H), 5.26-5.11 (m, 1H), 4.53-4.37 (m, 2H), 4.27-4.16 (m, 1H), 4.10-4.04 (m, 1H), 3.23 (s, 3H).

Preparation of (3): Intermediate 3 was prepared by prepared according to reaction condition described in reference Helvetica Chimica Acta, 2004, 87.2812. To a solution of 2 (9.2 g, 28.3 mmol) in dry DMSO (130.0 mL). DMTrSH (14.31 g, 42.5 mmol) was added, followed by tetramethylguanidine (3.6 g, 31.2 mmol) was added under N₂, reaction was stirred at r.t. for 3 h, LCMS showed 2 was consumed completely. 100.0 mL H₂O was added and extracted with EA (100.0 mL*2), organic phase was concentrated to give crude which was purified by column chromatography (SiO2, PE/EA=5:1 to 1:1) to give 3 (12.0 g, 97% purity) as a white solid. ESI-LCMS: m/z 563.2 [M−H]⁻; ¹H-NMR (400 MHz, DMSO-d₆): δ 11.43-11.42 (d, J=4.0 Hz, 1H), 7.57-7.55 (d, J=8.0 Hz, 1H), 7.33-7.17 (m, 9H), 6.89-6.86 (m, 4H), 5.80-5.74 (m, 1H), 5.65-5.62 (m, 1H), 5.58-5.57 (d, J=4.0 Hz, 1H), 5.16-5.01 (m, 1H), 3.98-3.90 (m, 1H), 3.73 (s, 6H), 3.73-3.67 (m, 1H), 2.50-2.37 (m, 2H).

Preparation of Example 10 monomer: To a solution of 3 (10.0 g, 17.7 mmol) in dichloromethane (120.0 mL) with an inert atmosphere of nitrogen was added CEOP[N(iPr)₂]₂ (6.4 g, 21.2 mmol) and DCI (1.8 g, 15.9 mmol) in order at room temperature. The resulting solution was stirred for 1.0 h at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in to give Example 10 monomer (12.8 g, 98% purity, 93% yield) as an oil. ESI-LCMS: m/z 765.2 [M+H]⁺; ¹H-NMR (400 MHz, DMSO-d₆): δ 11.44 (s, 1H), 7.70-7.66 (m, 1H), 7.32-7.18 (m, 9H), 6.89-6.85 (m, 4H), 5.80-5.64 (m, 2H), 5.38-5.22 (m, 1H), 4.38-4.15 (m, 1H), 3.81-3.70 (m, 8H), 3.61-3.43 (m, 3H), 2.76-2.73 (m, 1H), 2.66-2.63 (m, 1H), 2.50-2.41 (m, 2H), 1.12-1.05 (m, 9H), 0.97-0.95 (m, 3H); ³¹P-NMR (162 MHz, DMSO-d₆): δ 149.01, 148.97, 148.74, 148.67; ¹⁹F-NMR (376 MHz, DMSO-d₆): δ 149.01, 148.97, 148.74, 148.67.

### Example 11. Synthesis of Monomer

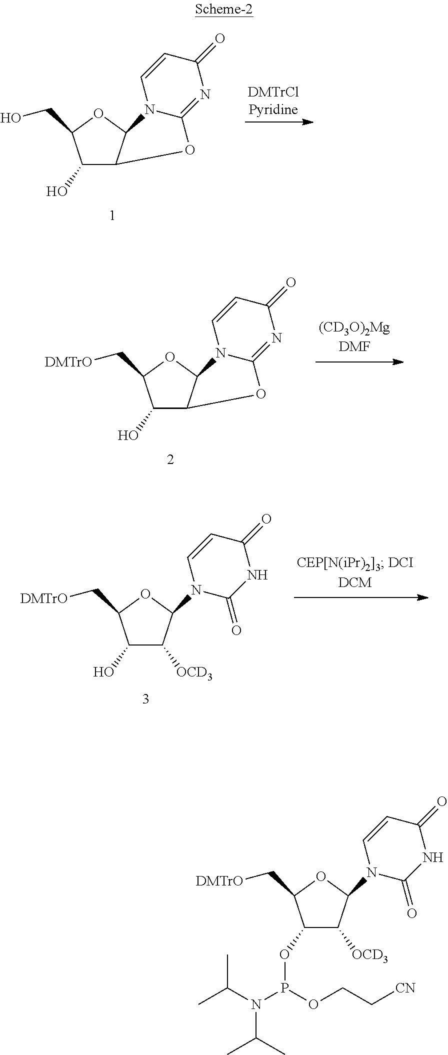

Preparation of (2): To a stirred solution of 1 (2.0 g, 8.8 mmol) in pyridine (20 mL) were added DMTrCl (3.3 g, 9.7 mmol) at r.t. The reaction mixture was stirred at r.t. for 2.5 hrs. With ice-bath cooling, the reaction was quenched with water and the product was extracted with EA (100 mL). The organic phase was evaporated to dryness under reduced pressure to give a residue which was purified by silica gel column chromatography (eluent, DCM:MeOH=50:1˜20:1) to give 2 (3.7 g, 7.2 mmol, 80.1%) as a white solid. ESI-LCMS: m/z 527 [M−H].

Preparation of (3): To the solution of 2 (2.8 g, 5.3 mmol) in dry DMF (56 mL) was added (CD₃₀)₂Mg (2.9 g, 31.8 mmol) at r.t. under N₂ atmosphere. The reaction mixture was stirred at 100° C. for 15 hrs. With ice-bath cooling, the reaction was quenched with saturated aq. NH₄C1 and extracted with EA (300 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 3 (2.0 g, 3.6 mmol, 67.9%) as a white solid. ESI-LCMS: m/z 562 [M−H]⁻; ¹H-NMR (400 MHz, DMSO-d₆): δ 11.38 (s, 1H), 7.73 (d, J=8 Hz, 1H), 7.46-7.19 (m, 9H), 6.91 (d, J=7.4 Hz, 4H), 5.81-5.76 (AB, J=20 Hz, 1H), 5.30 (d, J=8 Hz, 1H), 5.22 (s, 1H), 4.25-4.15 (m, 1H), 3.99-3.92 (m, 1H), 3.85-3.79 (m, 1H), 3.74 (s, 6H), 3.34-3.18 (m, 31H).

Preparation of Example 11 monomer: To a suspension of 3 (2.0 g, 3.5 mmol) in DCM (20 mL) was added DCI (357 mg, 3.0 mmol) and CEP[N(iPr)₂]₂ (1.3 g, 4.3 mmol). The mixture was stirred at r.t. for 1 h. LC-MS showed 3 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 11 monomer (2.1 g, 2.7 mmol, 77.1%) as a white solid. ESI-LCMS: m/z 764 [M+H]⁺; ¹H-NMR (400 MHz, ACN-d₃): δ 9.45-8.90 (m, 1H, exchanged with D₂O), 7.88-7.66 (m, 1H), 7.50-7.18 (m, 9H), 6.93-6.80 (m, 4H), 5.85 (d, J=8.2 Hz, 1H), 5.29-5.16 (m, 1H), 4.57-4.37 (m, 1H), 4.18-4.09 (m, 1H), 3.98-3.90 (m, 1H), 3.90-3.74 (m, 7H), 3.74-3.50 (m, 3H), 3.48-3.31 (m, 2H), 2.70-2.61 (m, 1H), 2.56-2.46 (m, 1H), 1.24-1.12 (m, 9H), 1.09-0.99 (m, 3H). ³¹P-NMR (162 MHz, ACN-d₃): δ=149.87, 149.55.

### Example 12. Synthesis of Monomer

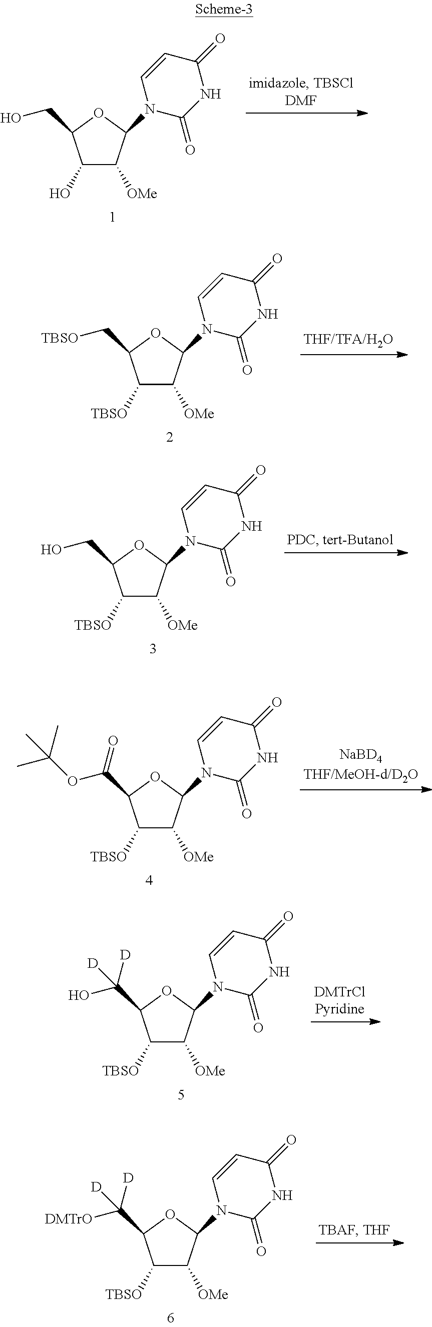

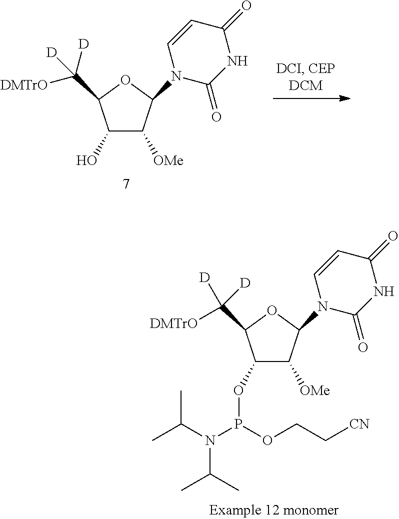

Preparation of (2): To the solution of 1 (39.2 g, 151.9 mmol) in DMF (390.0 mL) was added imidazole (33.0 g, 485.3 mmol) and TBSCl (57.2 g, 379.6 mmol) at 0° C. The reaction mixture was stirred at room temperature for 15 hrs under N₂ atmosphere. After addition of water, the resulting mixture was extracted with EA (500.0 mL). The combined organic layer was washed with water and brine, dried over Na₂SO₄, concentrated to give the crude 2 (85.6 g) as a white solid which was used directly for next step. ESI-LCMS: m/z 487.7 [M+H]⁺.

Preparation of (3): A solution of crude 2 (85.6 g) in a mixture solvent of TFA/H₂O=1/1 (400.0 mL) and THF (400.0 mL) was stirred at 0° C. for 30 min. After completion of reaction, the resulting mixture was added con·NH₃*H₂O to pH=7, and then extracted with EA (500.0 mL). The organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 3 (36.6 g, 98.4 mmol, 64.7% over two step) as a white solid. ESI-LCMS: m/z 372.5 [M+H]⁺; ¹H-NMR (400 MHz, DMSO-d₆): δ 11.36 (d, J=1 Hz, 1H), 7.92 (d, J=8 Hz, 1H), 5.83 (d, J=5 Hz, 1H), 5.67-5.65 (m, 1H), 5.19 (s, 1H), 4.30 (t, J=5 Hz, 1H), 3.85-3.83 (m, 2H), 3.68-3.52 (m, 2H), 0.88 (s, 9H), 0.09 (s, 6H).

Preparation of (4): To the solution of 3 (36.6 g, 98.4 mmol) in dry DCM (200.0 mL) and DMF (50.0 mL) was added PDC (73.9 g, 196.7 mmol), tert-butyl alcohol (188.0 mL) and Ac₂O (93.0 mL) at r.t under N₂ atmosphere, the reaction mixture was stirred at r.t for 2 hrs. The solvent was removed to give a residue which was purified by silica gel column chromatography (eluent, PE/EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 4 (24.3 g, 54.9 mmol, 55.8%) as a white solid. ESI-LCMS: m/z 443.2 [M+H]⁺; ¹H-NMR (400 MHz, DMSO-d₆): δ 11.30 (d, J=1 Hz, 1H), 7.92 (d, J=8 Hz, 1H), 5.86 (d, J=6 Hz, 1H), 5.67-5.65 (m, 1H), 4.33-4.31 (m, 1H), 4.13 (d, J=3 Hz, 1H), 3.73-3.70 (m, 1H), 1.34 (s, 9H), 0.77 (s, 9H), 0.08 (s, 6H).

Preparation of (5): To the solution of 4 (18.0 g, 40.7 mmol) in dry THF/MeOD/D₂O=10/2/1 (145.0 mL) was added NaBD₄ (5.1 g, 122.1 mmol) three times during an hour at 50° C., the reaction mixture was stirred at r.t. for 2 hrs. After completion of reaction, adjusted pH value to 7 with CH₃COOD, after addition of water, the resulting mixture was extracted with EA (300.0 mL). The combined organic layer was washed with water and brine, dried over Na₂SO₄, concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 5 (10.4 g, 27.8 mmol, 68.3%) as a white solid. ESI-LCMS: m/z 375.2 [M+H]⁺; ¹H-NMR (400 MHz, DMSO-d₆): δ 11.36 (d, J=1 Hz, 1H), 7.92 (d, J=8 Hz, 1H), 5.83 (d, J=5 Hz, 1H), 5.67-5.65 (m, 1H), 5.19 (s, 1H), 4.30 (t, J=5 Hz, 1H), 3.85-3.83 (m, 2H), 0.88 (s, 9H), 0.09 (s, 6H).

Preparation of (6): To a stirred solution of 5 (10.4 g, 27.8 mmol) in pyridine (100.0 mL) was added DMTrCl (12.2 g, 36.1 mmol) at r.t., The reaction mixture was stirred at r.t. for 2.5 hrs, the reaction was quenched with water and extracted with EA (200.0 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (13.5 g, 19.9 mmol, 71.6%) as a white solid. ESI-LCMS: m/z 677.8 [M+H]⁺; ¹H-NMR (400 MHz, DMSO-d₆): δ 11.39 (d, J=1 Hz, 1H), 7.86 (d, J=4 Hz, 1H), 7.35-7.21 (m, 9H), 6.90-6.88 (m, 4H), 5.78 (d, J=2 Hz, 1H), 5.30-5.27 (m, 1H), 4.33-4.30 (m, 1H), 3.91 (d, J=7 Hz, 1H), 3.85-3.83 (m, 1H), 3.73 (s, 6H), 3.38 (s, 3H), 0.77 (s, 9H), 0.03 (s, 3H), 0.01 (s, 3H).

Preparation of (7): To a solution of 6 (13.5 g, 19.9 mmol) in THF (130.0 mL) was added 1 M TBAF solution (19.0 mL). The reaction mixture was stirred at r.t. for 1.5 hrs. LC-MS showed 6 was consumed completely. Water (500.0 mL) was added and extracted with EA (300.0 mL), the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (10.9 g, 19.4 mmol, 97.5%) as a white solid. ESI-LCMS: m/z 563.6 [M+H]⁺; ¹H-NMR (400 MHz, DMSO-d₆): δ 11.39 (s, 1H), 7.23 (d, J=8 Hz, 1H), 7.73 (d, J=8 Hz, 1H), 7.36-7.23 (m, 9H), 6.90 (d, J=8 Hz, 4H), 5.81 (d, J=3 Hz, 1H), 5.30-5.28 (m, 1H), 5.22 (d, J=7 Hz, 1H), 4.20 (q, J=7 Hz, 1H), 3.93 (d, J=7 Hz, 1H), 3.81 (t, J=5 Hz, 1H), 3.74 (s, 6H), 3.41 (s, 3H).

Preparation of Example 12 monomer: To a suspension of 7 (10.9 g, 19.4 mmol) in DCM (100.0 mL) was added DCI (1.8 g, 15.7 mmol) and CEP[N(iPr)₂]₂ (6.1 g, 20.4 mmol). The mixture was stirred at r.t. for 1 h. LC-MS showed 7 was consumed completely. The mixture was washed with water twice and brine, dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 12 monomer (12.5 g, 14.5 mmol, 74.7%) as a white solid. ESI-LCMS: m/z 863.6 [M+H]⁺; ¹H-NMR (400 MHz, DMSO-d₆): δ 11.39 (s, 1H), 7.81-7.55 (m, 1H), 7.40-7.22 (m, 9H), 6.92-6.87 (m, 4H), 5.83-5.80 (m, 1H), 5.32-5.25 (m, 1H), 4.46-4.34 (m, 1H), 4.10-3.98 (m, 2H), 3.84-3.73 (m, 7H), 3.60-3.50 (m, 3H), 3.42, 3.40 (s, 3H), 2.78 (t, J=6 Hz, 1H), 2.62-2.59 (m, 1H), 2.07 (s, 1H), 1.17-0.96 (m, 12H); ³¹P-NMR (162 MHz, DMSO-d₆): δ 149.37, 149.06.

### Example 13. Synthesis of Monomer

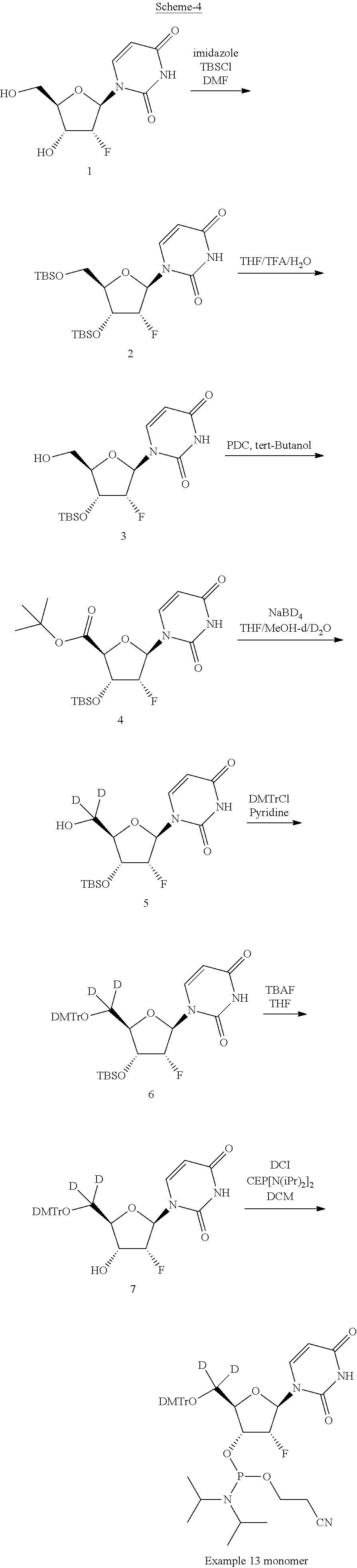

Preparation of (2): To the solution of 1 (13.0 g, 52.8 mmol) in DMF (100 mL) was added imidazole (12.6 g, 184.8 mmol) and TBSCl (19.8 g, 132.0 mmol) at 0° C., and the reaction mixture was stirred at room temperature for 15 h under N₂ atmosphere. After addition of water, the resulting product was extracted with EA (500 mL). The combined organic layer was washed with water and brine, dried over Na₂SO₄, and concentrated to give the crude 2 (30.6 g) as a white solid which was used directly for next step. ESI-LCMS: m/z 475 [M+H]⁺. WO2017106710A1

Preparation of (3): A solution of crude 2 (30.6 g) in a mixture solvent of TFA/H₂O=1/1 (100 mL) and THF (100 mL) was stirred at 0° C. for 30 min. After completion of reaction, the resulting mixture was added con·NH₃*H₂O to pH=7.5, and then the mixture was extracted with EA (500 mL), the organic layer was washed with brine, dried over Na₂SO₄ and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 3 (12.0 g, 33.3 mmol, 65.8% over two step) as a white solid. ESI-LCMS: m/z 361 [M+H]⁺; ¹H-NMR (400 MHz, DMSO-d₆): δ 11.39 (s, J=1 Hz, 1H, exchanged with D₂O), 7.88 (d, J=8 Hz, 1H), 5.91-5.86 (m, 1H), 5.66-5.62 (m, 1H), 5.21 (t, J=5.2 Hz, 1H, exchanged with D₂O), 5.18-5.03 (m, 1H), 4.37-4.29 (m, 1H), 3.87-3.83 (m, 1H), 3.78-3.73 (m, 1H), 3.56-3.51 (m, 1H), 0.87 (s, 9H), 0.09 (s, 6H). WO2017106710A1.

Preparation of (4): To the solution of 3 (11.0 g, 30.5 mmol) in dry DCM (60 mL) and DMF (15 mL) was added PDC (21. g, 61.0 mmol), tert-butyl alcohol (45 mL) and Ac₂O (32 mL) at r.t under N₂ atmosphere. And the reaction mixture was stirred at r.t for 2 h. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 4 (9.5 g, 22.0 mmol, 72.3%) as a white solid. ESI-LCMS: m/z 431 [M+H]⁺; ¹H-NMR (400 MHz, DMSO-d₆): δ 11.45 (s, J=1 Hz, 1H, exchanged with D₂O), 7.93 (d, J=8.5 Hz, 1H), 6.02-5.97 (m, 1H), 5.76-5.74 (m, 1H), 5.29-5.14 (m, 1H), 4.59-4.52 (m, 1H), 4.29-4.27 (m, 1H), 1.46 (s, 9H), 0.89 (s, 9H), 0.12 (s, 6H).

Preparation of (5): To the solution of 4 (8.5 g, 19.7 mmol) in dry THF/MeOD/D₂O=10/2/1 (80 mL) was added NaBD₄ (2.5 g, 59.1 mmol) three times per an hour at 50° C. And the reaction mixture was stirred at r.t for 2 h. After completion of reaction, adjusted pH value to 7 with CH₃COOD, after addition of water, the resulting mixture was extracted with EA (300 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 5 (3.5 g, 9.7 mmol, 50.3%) as a white solid. ESI-LCMS: m/z 363 [M+H]⁺; ¹H-NMR (400 MHz, DMSO-d₆): δ 11.41 (s, J=1 Hz, 1H, exchanged with D₂O), 7.88 (d, J=8 Hz, 1H), 5.91-5.86 (m, 1H), 5.66-5.62 (m, 1H), 5.19 (t, J=5.2 Hz, 1H, exchanged with D₂O), 5.18-5.03 (m, 1H), 4.37-4.29 (m, 1H), 3.87-3.83 (m, 1H), 0.88 (s, 9H), 0.10 (s, 6H).

Preparation of (6): To a stirred solution of 5 (3.4 g, 9.7 mmol) in pyridine (35 mL) were added DMTrCl (3.4 g, 10.1 mmol) at r.t. And the reaction mixture was stirred at r.t for 2.5 h. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (PCT Int. Appl., 2019173602), (5.5 g, 8.3 mmol, 85.3%) as a white solid. ESI-LCMS: m/z 665 [M+H]⁺; ¹H-NMR (400 MHz, DMSO-d₆): δ 11.50 (d, J=1 Hz, 1H, exchanged with D₂O), 7.92 (d, J=4 Hz, 1H), 7.44-7.27 (m, 9H), 6.96-6.93 (m, 4H), 5.94 (d, J=20.5 Hz, 1H), 5.39-5.37 (m, 1H), 5.32-5.17 (m, 1H), 4.60-4.51 (m, 1H), 4.01 (d, J=8.8 Hz, 1H), 3.80 (s, 6H), 0.80 (s, 9H), 0.09 (s, 3H), −0.05 (s, 3H).

Preparation of (7): To a solution of 6 (5.5 g, 8.3 mmol) in THF (50 mL) was added 1 M TBAF solution (9 mL). The reaction mixture was stirred at r.t. for 1.5 h. LC-MS showed 6 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (4.1 g, 7.5 mmol, 90.0%) as a white solid. ESI-LCMS: m/z 551 [M+H]⁺; ¹H-NMR (400 MHz, DMSO-d₆): δ 11.42 (s, 1H, exchanged with D₂O), 7.76 (d, J=8.2 Hz, 1H), 7.39-7.22 (m, 9H), 6.90-6.88 (m, 4H), 5.83 (d, J=20.5 Hz, 1H), 5.65 (d, J=7.0 Hz, 1H, exchanged with D₂O), 5.29 (d, J=7.2 Hz, 1H), 5.18-5.03 (m, 1H), 4.40-4.28 (m, 1H), 4.01 (d, J=8.8 Hz, 1H), 3.74 (s, 6H).

Preparation of Example 13 monomer: To a suspension of 7 (4.1 g, 7.5 mmol) in DCM (40 mL) was added DCI (0.7 g, 6.4 mmol) and CEP[N(iPr)₂]₂ (2.9 g, 9.7 mmol). The mixture was stirred at r.t. for 1 h. LC-MS showed 7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 13 monomer (5.0 g, 6.6 mmol, 90.0%) as a white solid. ESI-LCMS: m/z 751 [M+H]⁺; ¹H-NMR (400 MHz, DMSO-d₆): δ 11.43 (s, 1H), 7.85-7.82 (m, 1H), 7.40-7.23 (m, 9H), 6.90-6.85 (m, 4H), 5.94-5.86 (m, 1H), 5.40-5.24 (m, 2H), 4.74-4.49 (m, 1H), 4.12-4.09 (m, 2H), 3.79-3.47 (m, 1OH), 2.78-2.59 (m, 2H), 1.14-0.93 (m, 12H) ³¹P-NMR (162 MHz, DMSO-d₆): δ 149.67, 149.61, 149.32, 149.27.

### Example 14. Synthesis of Monomer

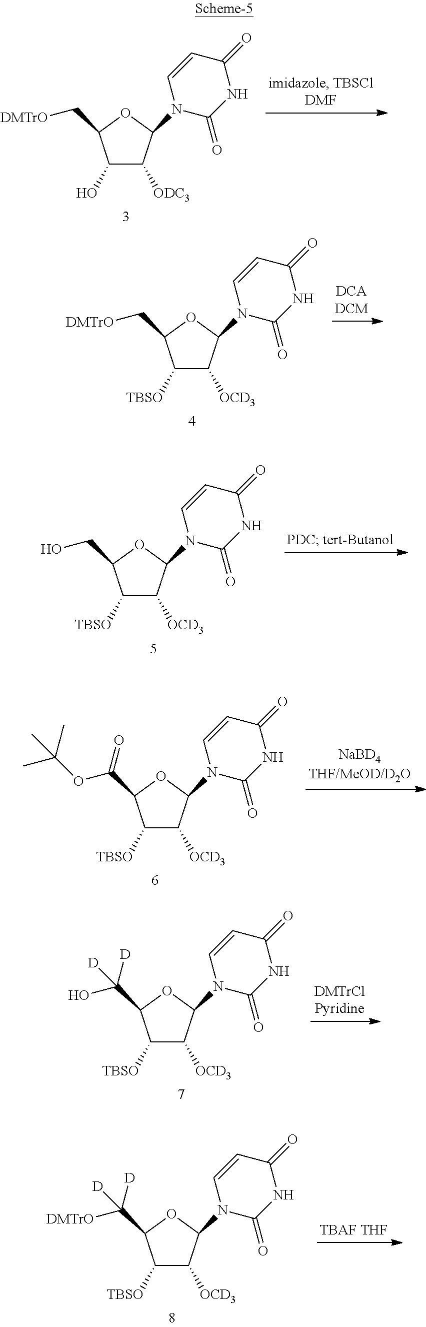

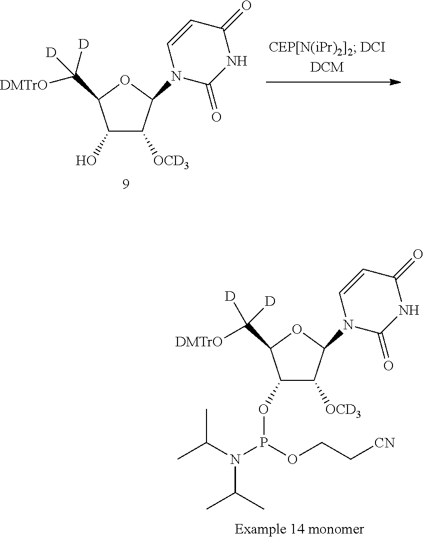

Preparation of (4): To the solution of 3 (14.3 g, 25.4 mmol, Scheme 2) in pyridine (150 mL) was added imidazole (4.5 g, 66.6 mmol) and TBSCl (6.0 g, 40.0 mmol) at 0° C., and the reaction mixture was stirred at room temperature for 15 h under N₂ atmosphere. After addition of water, the resulting mixture was extracted with EA (500 mL). The combined organic layer was washed with water and brine, dried over Na₂SO₄, and concentrated to give the crude 4 (18.0 g) as a white solid which was used directly for next step. ESI-LCMS: m/z 676 [M−H]⁻.

Preparation of (5): To the solution of crude 4 (18.0 g) in the solution of DCA (6%) in DCM (200 mL) was added TES (50 mL) at r.t, and the reaction mixture was stirred at room temperature for 5-10 min. After completion of reaction, the resulting mixture was added pyridine to pH=7, and then the solvent was removed and the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 5 (6.5 g, 17.2 mmol, 67.7% for two step) as a white solid. ESI-LCMS: m/z 376 [M+H]⁺; ¹H-NMR (400 MHz, DMSO-d₆): δ 7.92 (d, J=8 Hz, 1H), 5.82 (d, J=5.2 Hz, 1H), 5.68-5.63 (m, 1H), 5.20-5.15 (m, 1H), 4.32-4.25 (m, 1H), 3.87-3.80 (m, 2H), 3.69-3.61 (m, 1H), 3.57-3.49 (m, 1H), 0.88 (s, 9H), 0.09 (s, 6H).

Preparation of (6): To the solution of 5 (6.5 g, 17.2 mmol) in dry DCM (35 mL) and DMF (9 mL) was added PDC (12.9 g, 34.3 mmol), tert-butyl alcohol (34 mL) and Ac₂O (17 mL) at r.t under N₂ atmosphere. And the reaction mixture was stirred at r.t for 2 hrs. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (5.5 g, 12.3 mmol, 70.1%) as a white solid. ESI-LCMS: m/z 446 [M+H]⁺; ¹H-NMR (400 MHz, DMSO-d₆): δ=11.29 (s, 1H), 7.91 (d, J=8.4 Hz, 1H), 5.85 (d, J=6.4 Hz, 1H), 5.71-5.61 (m, 1H), 4.35-4.28 (m, 1H), 4.12 (d, J=3.2 Hz, 1H), 3.75-3.67 (m, 1H), 1.33 (s, 9H), 0.76 (s, 9H), 0.00 (d, J=1.6 Hz, 6H).

Preparation of (7): To the solution of 6 (5.4 g, 12.1 mmol) in THF/MeOD/D₂O=10/2/1 (44 mL) was added NaBD₄ (1.5 g, 36.3 mmol) at r.t. and the reaction mixture was stirred at 50° C. for 2 hrs. After completion of reaction, adjusted pH value to 7 with CH₃COOD. Water was added, the resulting mixture was extracted with EA (500 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (2.6 g, 6.8 mmol, 56.1%) as a white solid. ESI-LCMS: m/z 378 [M+H]⁺; ¹H-NMR (400 MHz, DMSO-d₆): δ 11.35 (s, 1H), 7.91 (d, J=8.0 Hz, 1H), 5.82 (d, J=5.2 Hz, 1H), 5.69-5.60 (m, 1H), 5.14 (s, 1H), 4.34-4.20 (m, 1H), 3.88-3.76 (m, 2H), 0.87 (s, 9H), 0.08 (s, 6H).

Preparation of (8): To a stirred solution of 7 (2.6 g, 6.8 mmol) in pyridine (30 mL) were added DMTrCl (3.5 g, 10.3 mmol) at r.t. And the reaction mixture was stirred at r.t. for 2.5 hrs. With ice-bath cooling, the reaction was quenched with water and the product was extracted into EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (4.3 g, 6.3 mmol, 90.1%) as a white solid. ESI-LCMS: m/z 678 [M−H]⁻; ¹H-NMR (400 MHz, DMSO-d₆): δ 11.39 (s, 1H), 7.86 (d, J=8.0 Hz, 1H), 7.42-7.17 (m, 9H), 6.96-6.83 (m, 4H), 5.82-5.69 (m, 2H), 5.29 (d, J=8.4 Hz, 1H), 4.36-4.25 (m, 1H), 3.90 (d, J=7.2 Hz, 1H), 3.86-3.80 (m, 1H), 3.73 (s, 6H), 0.75 (s, 9H), 0.02 (s, 3H), −0.04 (s, 3H).

Preparation of (9): To a solution of 8 (4.3 g, 6.3 mmol) in THF (45 mL) was added 1 M TBAF solution (6 mL). The reaction mixture was stirred at r.t. for 1.5 hrs. LCMS showed 8 was consumed completely. Water (200 mL) was added. The product was extracted with EA (200 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 8 (3.5 g, 6.1 mmol, 90.1%) as a white solid. ESI-LCMS: m/z 678 [M−H]⁻; ¹H-NMR (400 MHz, DMSO-d₆): δ 11.38 (d, J=2.0 Hz, 1H), 7.23 (d, J=8.0 Hz, 1H), 7.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of Example 14 monomer: To a suspension of 9 (2.1 g, 3.7 mmol) in DCM (20 mL) was added DCI (373 mg, 3.1 mmol) and CEP[N(iPr)₂]₂ (1.3 g, 4.4 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 14 monomer (2.2 g, 3.5 mmol, 80%) as a white solid. ESI-LCMS: m/z 766 [M+H]⁺; ¹H-NMR (400 MHz, ACN-d₃): δ 9.65-8.86 (m, 1H, exchanged with D₂O), 7.93-7.68 (m, 1H), 7.52-7.19 (m, 9H), 6.94-6.78 (m, 4H), 5.95-5.77 (m, 1H), 5.31-5.17 (m, 1H), 4.61-4.37 (m, 1H), 4.20-4.07 (m, 1H), 4.01-3.51 (m, 1OH), 2.74-2.59 (m, 1H), 2.57-2.43 (m, 1H), 1.27-1.10 (m, 9H), 1.09-0.95 (m, 3H). ³¹P-NMR (162 MHz, ACN-d₃): δ=149.88, 149.55.

### Example 15. Synthesis of Monomer

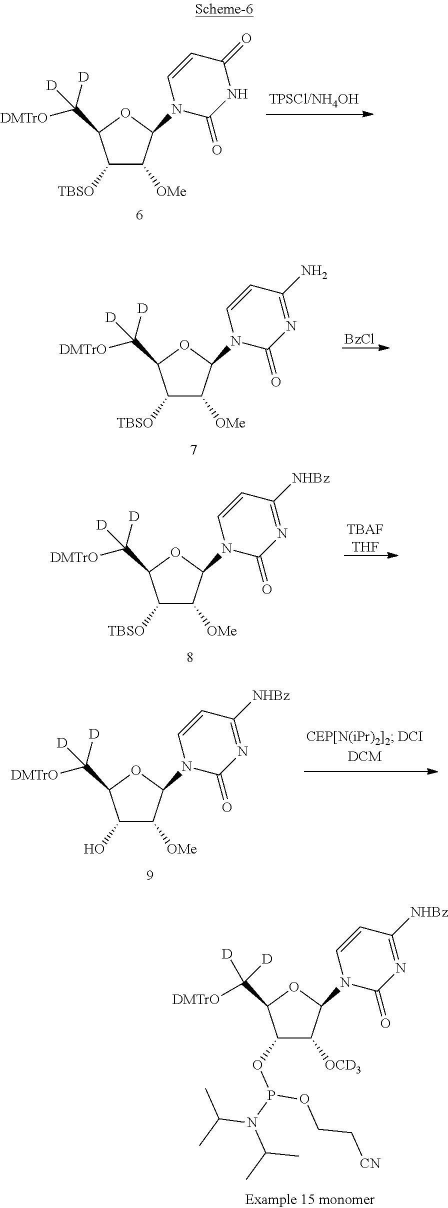

Preparation of (7): To a solution of 6 (17 g, 25.1 mmol, Scheme 3) in ACN (170 mL) was added DMAP (6.13 g, 50.3 mmol) and TEA (5.1 g, 50.3 mmol, 7.2 mL), Then added TPSCl (11.4 g, 37.7 mmol) at 0° C. under N₂ atmosphere and the mixture was stirred at r.t. for 3 h under N₂ atmosphere. Then con. NH₃·H₂O (27.3 g, 233.7 mmol) was added at r.t. and the mixture was stirred at r.t. for 16 h. The reaction was quenched with water and the product was extracted with EA (200 mL). The organic phase was concentrated to give the crude 7 (17.0 g) as a white solid which was used directly for next step.

Preparation of (8): To a stirred solution of 7 (17.0 g, 25.1 mmol) in pyridine (170 mL) were added BzCl (4.3 g, 30.1 mmol) 0° C. under N₂ atmosphere. And the reaction mixture was stirred at r.t for 2.5 h. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (19.0 g, 24.3 mmol, 95.6% over two step) as a white solid. ESI-LCMS: m/z 780 [M+H]⁺.

Preparation of (9): To a solution of 8 (19.0 g, 24.3 mmol) in THF (190 mL) was added 1 M TBAF solution (24 mL). The reaction mixture was stirred at r.t. for 1.0 h. LC-MS showed 8 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 9 (15.2 g, 23.1 mmol, 95.5%) as a white solid. ESI-LCMS: m/z 666 [M+H]⁺; ¹H-NMR (400 MHz, DMSO-d₆): δ 11.28 (s, 1H), 8.41 (m, 1H), 8.00-7.99 (m, 2H), 7.63-7.15 (m, 13H), 6.93-6.89 (m, 4H), 5.87 (s, 1H), 5.20 (d, J=7.4 Hz, 1H), 4.30 (m, 1H), 4.02 (m, 1H), 3.75 (s, 7H), 3.53 (s, 3H).

Preparation of Example 15 monomer: To a suspension of 9 (10.0 g, 15.0 mmol) in DCM (100 mL) was added DCI (1.5 g, 12.7 mmol) and CEP[N(iPr)₂]₂ (5.4 g, 18.0 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 15 monomer (11.5 g, 13.5 mmol, 90.7%) as a white solid. ESI-LCMS: m/z 866 [M+H]^{m}; ¹H-NMR (400 MHz, DMSO-d₆): δ=11.28 (s, 1H), 8.48-8.41 (m, 1H), 8.00-7.99 (m, 2H), 7.63-7.11 (m, 13H), 6.93-6.89 (m, 4H), 5.92 (m, 1H), 4.55-4.44 (m, 1H), 4.17 (m, 1H), 3.95 (m, 1H), 3.80-3.62 (m, 7H), 3.57-3.46 (m, 5H), 3.32 (s, 1H), 2.78 (m, 1H), 2.62-2.59 (m, 1H), 1.19-0.94 (m, 12H); ³¹P-NMR (162 MHz, DMSO-d₆): δ=149.52, 148.82.

### Example 16. Synthesis of Monomer

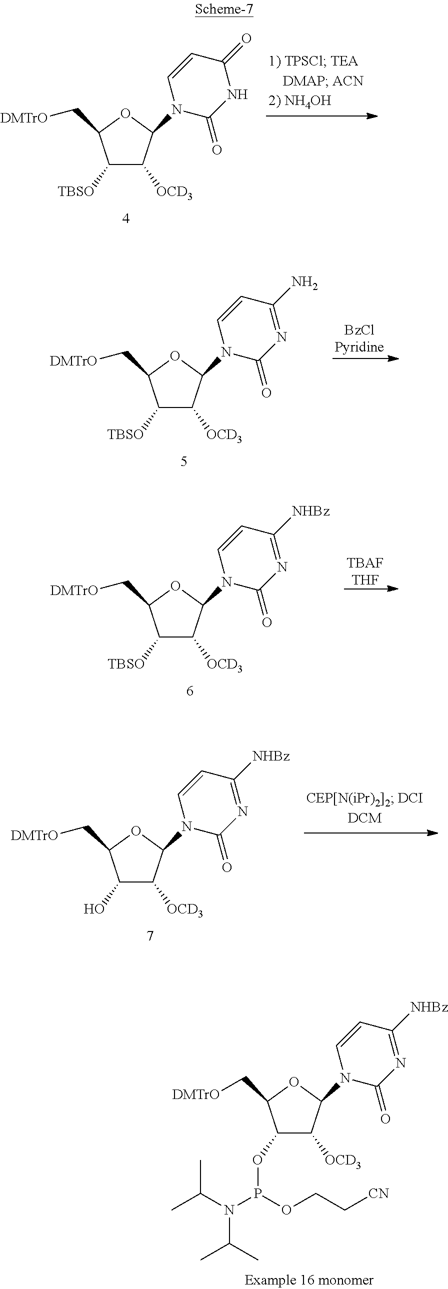

Preparation of (5): To the solution of 4 (18.8 g, Scheme 5) in dry ACN (200 mL) was added TPSCl (16.8 g, 65.2 mmol) and TEA (5.6 g, 65.2 mmol) and DMAP (6.8 g, 65.2 mmol), and the reaction mixture was stirred at room temperature for 3.5 hrs under N₂ atmosphere. After addition of water, the resulting mixture was extracted with EA (300 mL). The combined organic layer was washed with water and brine, dried over Na₂SO₄, and concentrated to give the crude 5 (22.0 g) as a white solid which was used directly for next step. ESI-LCMS: m/z 677 [M−H]⁺.

Preparation of (6): To a solution of 5 (22.0 g) in pyridine (150 mL) was added BzCl (6.8 g, 48.9 mmol) under ice bath. The reaction mixture was stirred at r.t. for 2.5 hrs. LCMS showed 5 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give the crude 6 (20.8 g, 26.7 mmol, 82% yield over two steps) as a white solid. ESI-LCMS: m/z 781 [M+H]⁺; ¹H-NMR (400 MHz, DMSO-d₆): δ 11.30 (s, 1H), 8.55 (d, J=8.0 Hz, 1H), 8.00-7.98 (m, 2H), 7.74-7.66 (m, 1H), 7.60-7.50 (m, 2H), 7.47-7.31 (m, 4H), 7.30-7.2 (m, 5H), 7.20-7.1 (m, 1H), 6.91 (d, J=7.4 Hz, 4H), 5.91-5.86 (AB, J=20.0 Hz, 1H), 4.30 (d, J=8.0 Hz, 1H), 3.87-3.78 (s, 1H), 3.78-3.70 (m, 6H), 3.62-3.51 (m, 1H), 3.28-3.2 (m, 1H), 2.15-2.05 (m, 3H), 0.73 (s, 9H), 0.00 (m, 6H).

Preparation of (7): To a solution of 6 (20.8 g, 26.7 mmol) in THF (210 mL) was added 1 M TBAF solution (32 mL). The reaction mixture was stirred at r.t. for 1.5 hrs. LCMS showed 6 was consumed completely. Water (600 mL) was added. The product was extracted with EA (400 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (12.4 g, 18.6 mmol, 70%) as a white solid. ESI-LCMS: m/z 667 [M+H]⁺; ¹H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 7H), 2.57-2.42 (m, 2H).

Preparation of Example 16 monomer: To a suspension of 7 (12.4 g, 18.6 mmol) in DCM (120 mL) was added DCI (1.7 g, 15.8 mmol) and CEP[N(iPr)₂]₂ (7.3 g, 24.2 mmol). The mixture was stirred at r.t. for 2 hrs. LC-MS showed 7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 16 monomer (13.6 g, 15.7 mmol, 84.0%) as a white solid. ESI-LCMS: m/z 867 [M+H]⁺; ¹H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 1OH), 2.74-2.59 (m, 1H), 2.57-2.43 (m, 1H), 1.27-1.10 (m, 9H), 1.09-0.95 (m, 3H). ³¹P-NMR (162 MHz, DMSO-d₆): δ 149.59, 148.85.

### Example 17. Synthesis of Monomer

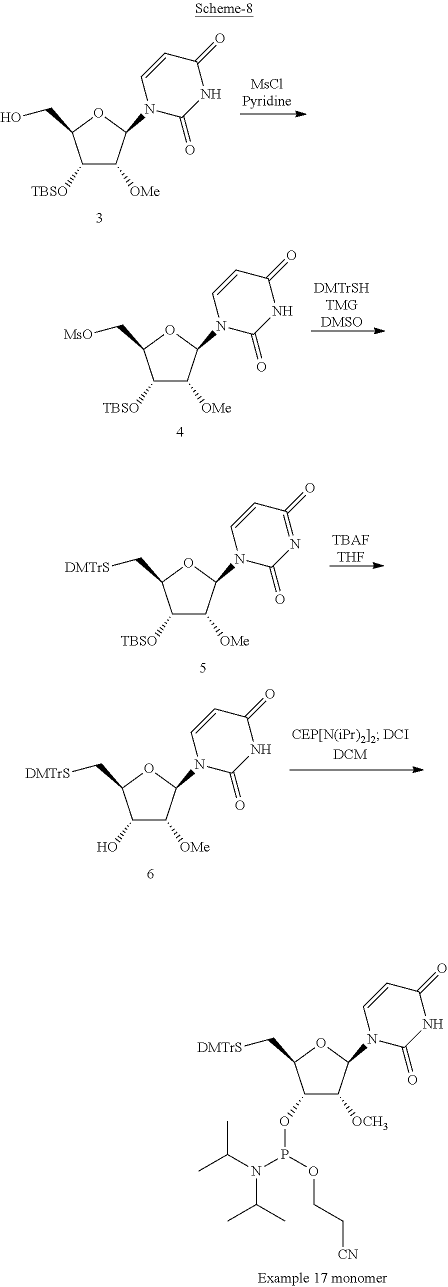

Preparation of (4): To a solution of 3 (13.1 g, 35.2 mmol, Scheme 3) in pyridine (130 mL) was added MsCl (4.8 g, 42.2 mmol) under −10˜0° C. The reaction mixture was stirred at r.t. for 2.5 h under N₂ atmosphere. TLC (DCM/MeOH=15:1) showed the reaction was consumed. The mixture was diluted with EA and water was added. The product was extracted with EA. The organic layer was washed with brine and dried over Na₂SO₄ and concentrated to give the crude. This resulted in to give the product 4 (14.2 g) which was used directly for the next step. ESI-LCMS: m/z 451 [M+H]⁺; ¹H-NMR (400 MHz, DMSO-d₆) δ 11.43 (m, 1H), 7.67-7.65 (m, 1H), 5.90-5.80 (m, 1H), 5.75-5.64 (m, 1H), 4.52-4.21 (m, 3H), 4.12-3.90 (m, 2H), 3.48-3.21 (m, 6H), 0.95-0.78 (s, 9H), 0.13-0.03 (s, 6H).

Preparation of (5): To a solution of 4 (14.2 g) in DMSO (200 mL) was added DMTrSH (19.6 g, 63.2 mmol) and tetramethylguanidine (5.1 g, 47.4 mmol) at r.t. The reaction mixture was stirred at r.t. for 3.5 h under N₂ atmosphere. LCMS showed 4 the reaction was consumed. The mixture was diluted with EA and water was added. The product was extracted with EA. The organic layer was washed with brine and dried over Na₂SO₄ and concentrated to give the crude. The crude was purified by silica gel column (SiO2, PE/EA=10:1˜1:1) to give 5 (14.2 g, 20.6 mmol, 58.5% yield over two steps) as a white solid. ESI-LCMS: m/z 689 [M+H]; H-NMR (400 MHz, DMSO-d₆) δ 11.39 (m, 1H), 7.63-7.61 (d, J=8.0 Hz, 1H), 7.45-7.1 (m, 9H), 6.91-6.81 (m, 4H), 5.80-5.70 (m, 2H), 4.01-3.91 (m, 1H), 3.85-3.78 (m, 1H), 3.78-3.65 (m, 6H), 3.60-3.51 (m, 1H), 3.43-3.2 (m, 3H), 2.50-2.32 (m, 2H), 0.95-0.77 (s, 9H), −0.00-0.02 (s, 6H).

Preparation of (6): To a solution of 5 (14.2 g, 20.6 mmol) in THF (140 mL) was added 1 M TBAF solution (20 mL). The reaction mixture was stirred at r.t. under N₂ atmosphere for 2.5 h. LCMS showed 5 was consumed completely. Water was added. The product was extracted with EA and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 6 (10.5 g, 18.2 mmol, 88.5%) as a white solid. ESI-LCMS: m/z 576 [M+H]⁺; ¹H-NMR (400 MHz, DMSO-d₆) δ 11.38 (m, 1H), 7.56-7.54 (d, J=8.0 Hz, 1H), 7.45-7.1 (m, 9H), 6.91-6.81 (m, 4H), 5.80-5.70 (m, 2H), 4.05-4.00 (m, 1H), 3.81-3.79 (m, 1H), 3.74 (m, 2H), 3.78-3.65 (m, 6H), 3.60-3.51 (m, 1H), 3.43-3.2 (m, 3H), 2.40-2.32 (m, 1H).

Preparation of Example 17 monomer: To a suspension of 9 (10.5 g, 18.2 mmol) in DCM (100 mL) was added DCI (1.7 g, 15.5 mmol) and CEP[N(iPr)₂]₂ (7.2 g, 23.7 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 17 monomer (12.5 g, 16.1 mmol, 88%) as a white solid. ESI-LCMS: m/z 776 [M+H]⁺; ¹H-NMR (400 MHz, DMSO-d₆) δ 11.41 (m, 1H), 7.64-7.59 (m, 1H), 7.40-7.25 (m, 4H), 7.25-7.10 (m, 5H), 6.89-6.86 (m, 4H), 5.72-5.67 (m, 2H), 4.02-4.00 (m, 2H), 3.76-3.74 (m, 8H), 3.74-3.73 (m, 3H), 3.51-3.49 (d, J=8 Hz, 1H), 3.33-3.29 (m, 1H), 2.77-2.73 (m, 1H), 2.63-2.60 (m, 1H), 2.50-2.47 (m, 1H), 1.12-0.99 (m, 12H). ³¹P-NMR (162 MHz, DMSO-d₆): δ 148.92, 148.84.

### Example 18. Synthesis of Monomer

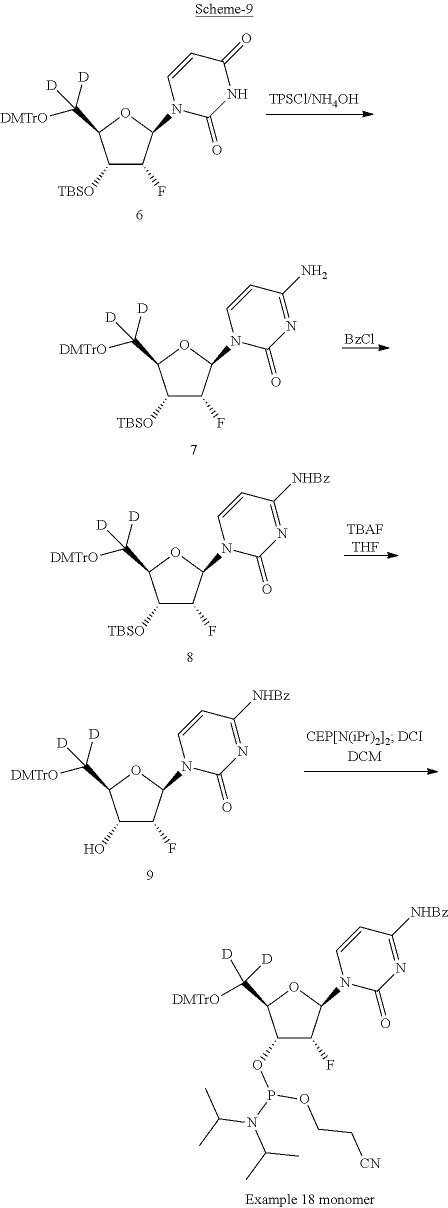

Preparation of (7): To a solution of 6 (16 g, 24.1 mmol, Scheme 4) in ACN (160 mL) was added DMAP (5.9 g, 48.2 mmol) and TEA (4.8 g, 48.2 mmol), then added TPSCl (10.9 g, 36.1 mmol) at 0° C. under N₂ atmosphere and the mixture was stirred at r.t. for 5 hrs under N₂ atmosphere. Then con. NH₃·H₂O (30 mL) was added at r.t. and the mixture was stirred at r.t. for 16 h. The reaction was quenched with water and the product was extracted with EA (200 mL). The organic phase was concentrated to give the crude 7 (16.0 g) as a white solid which was used directly for next step.

Preparation of (8): To a stirred solution of 7 (16.0 g, 24.1 mmol) in pyridine (160 mL) were added BzCl (4.1 g, 28.9 mmol) 0° C. under N₂ atmosphere. And the reaction mixture was stirred at r.t. for 2.5 h. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (18.0 g, 23.4 mmol, 97.0%) as a white solid. ESI-LCMS: m/z 768 [M+H]⁺; ¹H-NMR (400 MHz, DMSO-d₆): δ 11.31 (s, 1H), 8.47 (d, J=7.2 Hz, 1H), 7.99 (d, J=7.6 Hz, 2H), 7.65-7.16 (m, 13H), 6.92 (d, J=8.8 Hz, 4H), 6.01 (d, J=18.4 Hz, 1H), 5.18-5.04 (dd, 1H), 4.58-4.52 (m, 1H), 4.07 (d, J=9.6 Hz, 1H), 3.75 (s, 6H), 0.73 (s, 9H), 0.05 (s, 3H), −0.06 (s, 3H).

Preparation of (9): To a solution of 8 (18.0 g, 23.4 mmol) in THF (180 mL) was added 1 M TBAF solution (23 mL). The reaction mixture was stirred at r.t. for 1.5 h. LC-MS showed 8 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (13.7 g, 21.1 mmol, 90.5%) as a white solid. ESI-LCMS: m/z 654.2 [M+H]⁺; ¹H-NMR (400 MHz, DMSO-d₆): δ 11.31 (s, 1H), 8.35 (d, J=7.4 Hz, 1H), 8.01 (m, 2H), 7.65-7.16 (m, 13H), 6.92 (d, J=8.8 Hz, 4H), 5.94 (d, J=18.0 Hz, 1H), 5.71 (d, J=7.0 Hz, 1H), 5.12-4.98 (dd, 1H), 4.51-4.36 (m, 1H), 4.09 (d, J=9.6 Hz, 1H), 3.75 (s, 6H).

Preparation of Example 18 monomer: To a suspension of 9 (10.6 g, 16.2 mmol) in DCM (100 mL) was added DCI (1.6 g, 13.7 mmol) and CEP[N(iPr)₂]₂ (5.8 g, 19.4 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 18 monomer (10.5 g, 14.5 mmol, ⁷5.9%) as a white solid. ESI-LCMS: m/z 854.3 [M+H]⁺; ¹H-NMR (400 MHz, DMSO-d₆): δ 11.31 (s, 1H), 8.41-8.37 (m, 1H), 8.01 (d, J=7.7 Hz, 2H), 7.65-7.16 (m, 13H), 6.92-6.88 (m, 4H), 6.06-5.98 (m, 1H), 5.33-5.15 (m, 1H), 4.78-4.58 (m, 1H), 4.23-4.19 (m, 1H), 3.81-3.73 (m, 6H), 3.60-3.50 (m, 3H), 3.32 (s, 1H), 2.76 (t, J=6.0 Hz, 1H), 2.60 (t, J=5.8 Hz, 1H), 1.15-0.94 (m, 12H); ³¹P-NMR (162 MHz, DMSO-d₆): δ 150.23, 150.18, 149.43, 149.38.

### Example 19. Synthesis of Monomer

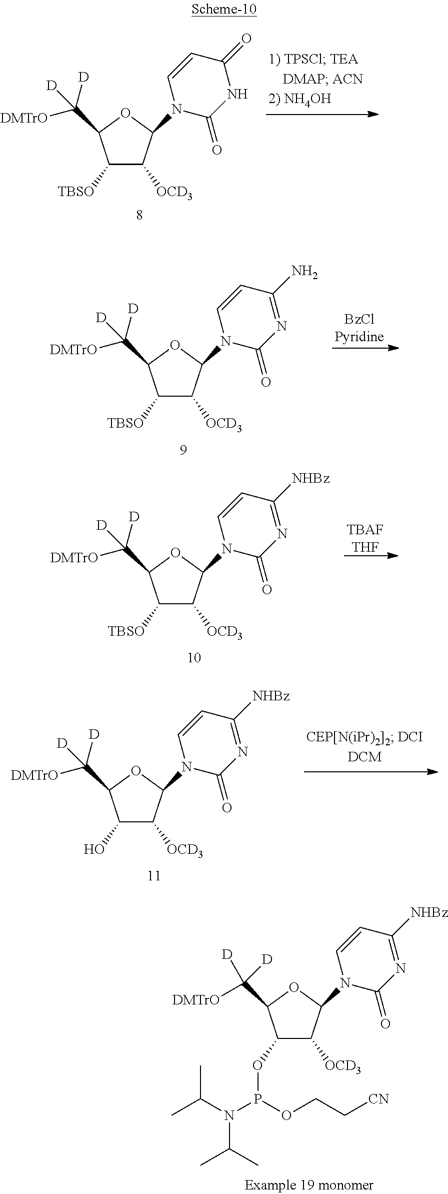

Preparation of (9): To a solution of 8 (18.8 g, 26.4 mmol, Scheme 5) in ACN (200 mL) was added TPSCl (16.8 g, 55.3 mmol) and DMAP (5.6 g, 55.3 mmol) and TEA (6.8 g, 55.3 mmol). The reaction mixture was stirred at r.t. for 3.5 hrs. LCMS showed the reaction was consumed. The mixture was diluted with con. NH₄OH (28 mL). The mixture was diluted with water and EA. The product was extracted with EA. The organic layer was washed with brine and dried over Na₂SO₄ and concentrated to give the crude 9 (18.5 g) which was used directly for the next step.

Preparation of (10): To a solution of 9 (18.8 g, 27.69 mmol) in pyridine (200 mL) was added BzCl (5.8 g, 41.5 mmol) under ice bath. The reaction mixture was stirred at r.t. for 2.5 hrs. LCMS showed 9 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give 10 (19.8 g, 25.3 mmol, 91% yield) as a white solid. ESI-LCMS: m/z 783 [M−H]⁻; ¹H-NMR (400 MHz, DMSO-d₆): δ 11.29 (d, J=2.0 Hz, 1H), 8.42 (d, J=8.0 Hz, 1H), 8.02-8.00 (m, 2H), 7.64-7.62 (m, 1H), 7.60-7.41 (m, 2H), 7.47.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of (11): To a solution of 10 (18.8 g, 26.4 mmol) in THF (190 mL) was added 1 M TBAF solution (28 mL). The reaction mixture was stirred at r.t. for 1.5 hrs. LCMS showed 10 was consumed completely. Water (200 mL) was added. The product was extracted with EA (200 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 11 (17.1 g, 25.6 mmol, 96%) as a white solid. ESI-LCMS: m/z 669 [M−H]⁻; ¹H-NMR (400 MHz, DMSO-d₆): δ 11.29 (d, J=2.0 Hz, 1H), 8.42 (d, J=8.0 Hz, 1H), 8.02-8.00 (m, 2H), 7.64-7.62 (m, 1H), 7.60-7.41 (m, 2H), 7.47.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of Example 19 monomer: To a suspension of 11 (10.8 g, 16.2 mmol) in DCM (100 mL) was added DCI (1.5 g, 13.7 mmol) and CEP[N(iPr)₂]₂ (5.8 g, 19.3 mmol). The mixture was stirred at r.t. for 2 hrs. LC-MS showed 11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 19 monomer (11.3 g, 13 mmol, 80%) as a white solid. ESI-LCMS: m/z 868 [M+H]⁺; ¹H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 1OH), 2.74-2.59 (m, 1H), 2.57-2.43 (m, 1H), 1.27-1.10 (m, 9H), 1.09-0.95 (m, 3H). ³¹P-NMR (162 MHz, DMSO-d₆): δ 149.52, 148.81.

### Example 20. Synthesis of Monomer

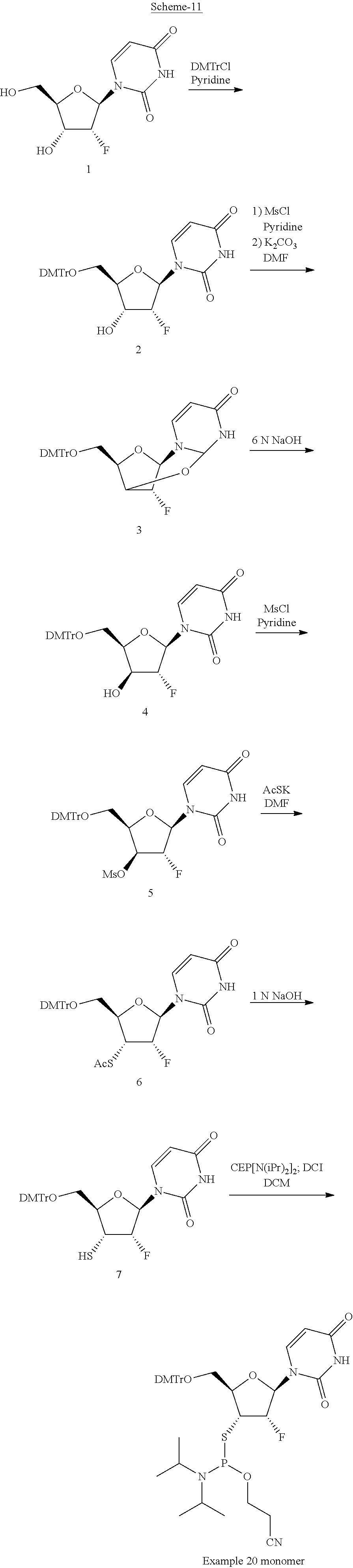

Preparation of (2): To a stirred solution of 1 (100.0 g, 406.5 mmol) in pyridine (1000 mL) were added DMTrCl (151.2 g, 447.1 mmol) at r.t. And the reaction mixture was stirred at r.t. for 2.5 hrs. With ice-bath cooling, the reaction was quenched with water and the product was extracted with EA (3000 mL). The organic phase was evaporated to dryness under reduced pressure to give a residue which was purified by silica gel column chromatography (SiO2, dichloromethane:methanol=100:1) to give 2 (210.0 g, 90%) as a white solid. ESI-LCMS: m/z 548.2 [M+H]⁺; ¹H-NMR (400 MHz, DMSO-d₆): δ 11.43 (d, J=1.8 Hz, 1H), 7.77 (d, J=8.0 Hz, 1H), 7.40-7.21 (m, 9H), 6.92-6.88 (m, 4H), 5.89 (d, J=20.0 Hz, 1H), 5.31-5.29 (m, 1H), 5.19-5.04 (dd, 1H), 4.38-4.31 (m, 1H), 4.02-3.98 (m, 1H), 3.74 (s, 6H), 3.30 (d, J=3.2 Hz, 2H); ¹⁹F-NMR (376 MHz, DMSO-d₆): δ −199.51.

Preparation of (3): To a stirred solution of 2 (100.0 g, 182.8 mmol) in pyridine (1000 mL) were added MsCl (31.2 g, 274.2 mmol) at 0° C. under N₂ atmosphere. And the reaction mixture was stirred at r.t for 2.5 h. With ice-bath cooling, the reaction was quenched with water and the product was extracted with EA (200 mL). The organic phase was evaporated to dryness under reduced pressure to give the crude (114.0 g) as a white solid which was used directly for next step. To the solution of the crude (114.0 g, 187.8 mmol) in DMF (2000 mL) was added K2CO3 (71.5 g, 548.4 mmol), and the reaction mixture was stirred at 90° C. for 15 h under N₂ atmosphere. After addition of water, the resulting mixture was extracted with EA (500 mL). The combined organic layer was washed with water and brine, dried over Na₂SO₄, and concentrated to give a residue which was purified by silica gel column chromatography (SiO2, dichloromethane:methanol=30:1) to give 3 (100.0 g, 90%) as a white solid. ESI-LCMS: m/z 531.2 [M+H]⁺; ¹H-NMR (400 MHz, DMSO-d₆): δ 7.79 (d, J=8.0 Hz, 1H), 7.40-7.21 (m, 9H), 6.89-6.83 (m, 4H), 6.14 (d, J=5.4 Hz, 1H), 6.02-5.90 (dd, 1H), 5.87 (d, J=20.0 Hz, 1H), 5.45 (m, 1H), 4.61 (m, 1H), 3.73 (d, J=1.9 Hz, 6H), 3.30-3.15 (m, 2H), 1.24-1.16 (m, 1H); ¹⁹F-NMR (376 MHz, DMSO-d₆): δ −204.23.

Preparation of (4): A solution of 3 (100 g, 187.8 mmol) in THF (1000 mL) was added 6N NaOH (34 mL, 206.5 mmol). The mixture was stirred at r.t. for 6 h. After completion of reaction, the resulting mixture was added H₂O, and then the mixture was extracted with EA, the organic layer was washed with brine, dried over sodium sulfate and removed to give the residue was purified by silica gel column chromatography (SiO2, dichloromethane:methanol=30:1) to give 4 (90.4 g, 90%) as a white solid. ESI-LCMS: m/z 548.2 [M+H]⁺; ¹⁹F-NMR (376 MHz, DMSO-d₆): δ −184.58.

Preparation of (5): To a stirred solution of 4 (90.4 g, 165.2 mmol) in pyridine (1000 mL) were added MsCl (61.5 g, 495.6 mmol) at 0° C. under N₂ atmosphere. And the reaction mixture was stirred at r.t for 16 hrs. With ice-bath cooling, the reaction was quenched with water and the product was extracted with EA. the organic layer was washed with brine, dried over sodium sulfate and removed to give the residue was purified by silica gel column chromatography (SiO2, PE:EA=1:1) to give 5 (75.0 g, 90%) as a white solid. ESI-LCMS: m/z 626.2 [M+H]⁺; ¹H-NMR (400 MHz, DMSO-d₆): δ 11.51 (d, J=1.6 Hz, 1H), 7.43-7.23 (m, 1OH), 6.92-6.88 (m, 4H), 6.08 (d, J=20.0 Hz, 1H), 5.55-5.39 (m, 2H), 4.59 (m, 1H), 3.74 (s, 6H), 3.48-3.28 (m, 2H), 3.17 (s, 3H); ¹⁹F-NMR (376 MHz, DMSO-d₆): δ −187.72.

Preparation of (6): To the solution of 5 (75.0 g, 120.4 mmol) in DMF (1500 mL) was added KSAc (71.5 g, 548.4 mmol) at 110° C. under N₂ atmosphere, After the reaction mixture was stirred at 110° C. for 3 h were added KSAc (71.5 g, 548.4 mmol) under N₂ atmosphere. And the reaction mixture was stirred at r.t for 16 h. After addition of water, the resulting mixture was extracted with EA. The combined organic layer was washed with water and brine, dried over Na₂SO₄, and concentrated to give a residue which was purified by silica gel column chromatography (SiO2, PE:EA=1:1) to give 6 (29.0 g, 90%) as a white solid. ESI-LCMS: m/z 605.2 [M+H]⁺; ¹H-NMR (400 MHz, DMSO-d₆): δ 11.45 (d, J=1.9 Hz, 1H), 7.95 (d, J=8.0 Hz, 1H), 7.38-7.21 (m, 9H), 6.92-6.87 (m, 4H), 5.93 (m, 1H), 5.50-5.36 (dd, 1H), 5.25-5.23 (dd, 1H), 4.54-4.42 (m, 1H), 4.17-4.12 (m, 1H), 3.74 (m, 7H), 3.35-3.22 (m, 2H), 2.39 (s, 1H); ¹⁹F-NMR (376 MHz, DMSO-d₆): δ −181.97.

Preparation of (7): A solution of 6 (22 g, 36.3 mmol) in a mixture solvent of THF/MeOH (1:1, 200 mL) was added 1N NaOMe (70 mL, 72.6 mmol) was stirred at 20° C. for 4 h. After completion of reaction, the resulting mixture was added H₂O, and then the mixture was extracted with EA, the organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 25 min, the eluted product was collected at CH₃CN/H₂O (0.5% NH₄HCO₃)=4/3; Detector, UV 254 nm. This resulted in to give 7 (10.5 g, 14.5 mmol, 75.9%) as a white solid. ESI-LCMS: m/z 565.1 [M+H]⁺; ¹H-NMR (400 MHz, DMSO-d₆): δ 11.45 (s, 1H), 7.83 (d, J=8.0 Hz, 1H), 7.40-7.23 (m, 9H), 6.90 (d, J=8.8 Hz, 4H), 5.88 (m, 1H), 5.29-5.15 (m, 2H), 3.72 (m, 7H), 3.43 (m, 2H), 2.78 (d, J=10.6 Hz, 1H).

Preparation of Example 20 monomer: To a suspension of 7 (10.5 g, 18.6 mmol) in DCM (100 mL) was added DCI (1.8 g, 15.7 mmol) and CEP[N(iPr)₂]₂ (6.7 g, 22.3 mmol). The mixture was stirred at r.t. for 1 h. LC-MS showed 8 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 20 monomer (10.5 g, 14.5 mmol, 75.9%) as a white solid. ESI-LCMS: m/z 765.3 [M+H]⁺; ¹H-NMR (400 MHz, DMSO-d₆): δ 11.40 (d, J=12.2 Hz, 1H), 7.90-7.86 (m, 1H), 7.41-7.24 (m, 9H), 6.91-6.89 (m, 4H), 5.97 (m, 1H), 5.33-5.10 (m, 2H), 4.18-4.16 (m, 1H), 3.91-3.39 (m, 17H), 2.81 (t, J=5.6 Hz, 1H), 2.66 (t, J=6.0 Hz, 1H), 1.33-0.97 (m, 12H); ³¹P-NMR (162 MHz, DMSO-d₆): δ 164.57, 160.13.

### Example 21. Synthesis of Monomer

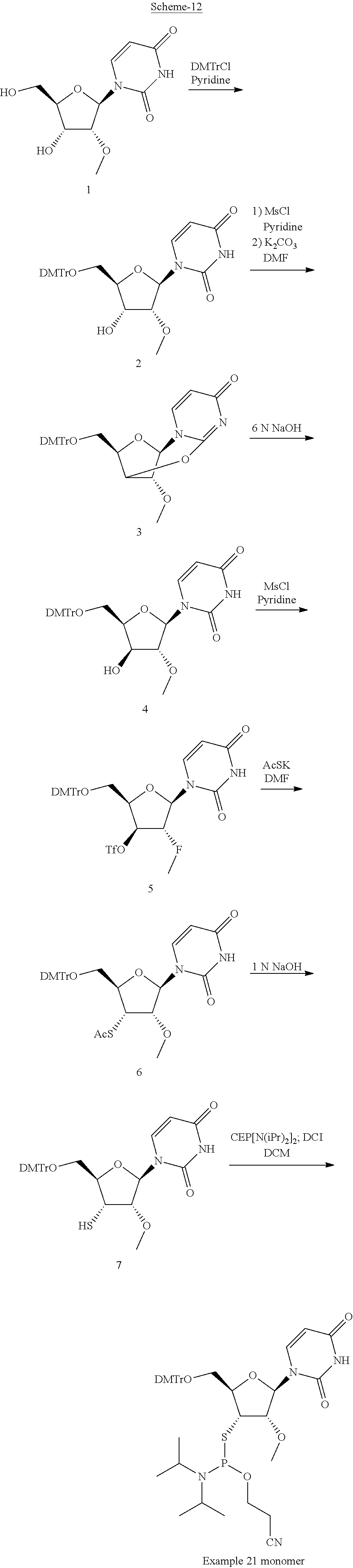

Preparation of (2): To a stirred solution of 1 (100.0 g, 387.5 mmol) in pyridine (1000 mL) was added DMTrCl (151.2 g, 447.1 mmol) at r.t. And the reaction mixture was stirred at r.t. for 2.5 hrs. With ice-bath cooling, the reaction was quenched with water and the product was extracted with EA (3000 mL). The organic phase was evaporated to dryness under reduced pressure to give a residue which was purified by silica gel column chromatography (SiO2, dichloromethane:methanol=100:1) to give 2 (200.0 g, 90%) as a white solid. ESI-LCMS: m/z 561 [M+H]⁺.

Preparation of (3): To a stirred solution of 2 (73.0 g, 130.3 mmol) in pyridine (730 mL) were added MsCl (19.5 g, 169.2 mmol) at 0° C. under N₂ atmosphere. And the reaction mixture was stirred at r.t for 2.5 h. With ice-bath cooling, the reaction was quenched with water and the product was extracted with EA (200 mL). The organic phase was evaporated to dryness under reduced pressure to give the crude (80.0 g) as a white solid which was used directly for next step. To the solution of the crude (80.0 g, 130.3 mmol) in DMF (1600 mL) was added K2CO3 (71.5 g, 390.9 mmol), and the reaction mixture was stirred at 90° C. for 15 h under N₂ atmosphere. After addition of water, the resulting mixture was extracted with EA (500 mL). The combined organic layer was washed with water and brine, dried over Na₂SO₄, and concentrated to give a residue which was purified by silica gel column chromatography (SiO2, dichloromethane:methanol=30:1) to give 3 (55.0 g, 90%) as a white solid. ESI-LCMS: m/z 543. [M+H]⁺; ¹H-NMR (400 MHz, DMSO-d₆): δ 7.68 (d, J=8.0 Hz, 1H), 7.40-7.21 (m, 9H), 6.89-6.83 (m, 4H), 5.96 (s, 1H), 5.83 (d, J=5.4 Hz, 1H), 5.26 (s, 1H), 4.59 (s, 1H), 4.46 (t, J=6.0 Hz, 1H), 3.72 (s, 6H), 3.44 (s, 3H), 3.18-3.12 (m, 2H).

Preparation of (4): A solution of 3 (55 g, 101.8 mmol) in THF (550 mL) was added 6N NaOH (34 mL, 206.5 mmol). The mixture was stirred at 20° C. for 6 hrs. After completion of reaction, the resulting mixture was added H₂O, and then the mixture was extracted with EA, the organic layer was washed with brine, dried over sodium sulfate and removed to give the residue was purified by silica gel column chromatography (SiO2, dichloromethane:methanol=30:1) to give 4 (57.4 g, 87%) as a white solid. ESI-LCMS: m/z 561 [M+H]⁺.

Preparation of (5): To a stirred solution of 4 (57.4 g, 101.8 mmol) in pyridine (550 mL) were added MsCl (61.5 g, 495.6 mmol) at 0° C. under N₂ atmosphere. And the reaction mixture was stirred at r.t for 16 h. With ice-bath cooling, the reaction was quenched with water and the product was extracted with EA. the organic layer was washed with brine, dried over sodium sulfate and removed to give the residue was purified by silica gel column chromatography (SiO2, PE:EA=1:1) to give 5 (57.0 g, 90%) as a white solid. ESI-LCMS: m/z 639 [M+H]⁺.

Preparation of (6): To the solution of 5 (57.0 g, 89.2 mmol) in DMF (600 mL) was added KSAc (71.5 g, 448.4 mmol) at 110° C. under N₂ atmosphere, After the reaction mixture was stirred at 110° C. for 3 h were added KSAc (71.5 g, 448.4 mmol) under N₂ atmosphere. And the reaction mixture was stirred at r.t for 16 h. After addition of water, the resulting mixture was extracted with EA. The combined organic layer was washed with water and brine, dried over Na₂SO₄, and concentrated to give a residue which was purified by silica gel column chromatography (SiO2, PE:EA=1:1) to give 6 (29.0 g, 47%) as a white solid. ESI-LCMS: m/z 619.2 [M+H]⁺; ¹H-NMR (400 MHz, DMSO-d₆): δ 11.41 (s, 1H), 8.06 (s, 1H), 7.40-7.23 (m, 9H), 6.90 (d, J=8.8 Hz, 4H), 5.82 (s, 1H), 5.10-5.08 (dd, 1H), 4.38-4.34 (m, 1H), 4.08-4.02 (m, 3H), 3.74 (s, 6H), 3.45 (s, 3H), 3.25 (m, 2H), 2.37 (s, 3H); ESI-LCMS: m/z 619 [M+H]⁺.

Preparation of (7): A solution of 6 (22 g, 35.3 mmol) in a mixture solvent of THF/MeOH (1:1, 200 mL) was added 1N NaOMe (70 mL, 72.6 mmol) was stirred at 20° C. for 4 h. After completion of reaction, the resulting mixture was added H₂O, and then the mixture was extracted with EA, the organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 25 min, the eluted product was collected at CH₃CN/H₂O (0.5% NH₄HCO₃)=4/3; Detector, UV 254 nm. This resulted in to give 7 (14.0 g, 70.9%) as a white solid. ESI-LCMS: m/z 576.1 [M+H]⁺; ¹H-NMR (400 MHz, DMSO-d₆): δ 11.38 (s, 1H), 7.90 (d, J=8.0 Hz, 1H), 7.40-7.23 (m, 9H), 6.90 (d, J=8.8 Hz, 4H), 5.80 (s, 1H), 5.15-5.13 (dd, 1H), 3.93 (m, 1H), 3.87 (d, J=5.0 Hz, 1H), 3.74 (s, 6H), 3.59 (m, 2H), 3.49 (s, 3H), 3.39 (d, J=2.2 Hz, 2H), 2.40 (d, J=10.2 Hz, 1H).

Preparation of Example 21 monomer: To a suspension of 7 (10.5 g, 18.6 mmol) in DCM (100 mL) was added DCI (1.8 g, 15.7 mmol) and CEP[N(iPr)₂]₂ (6.7 g, 22.3 mmol). The mixture was stirred at r.t. for 1 h. LC-MS showed 7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 21 monomer (10.5 g, 14.5 mmol, 75.9%) as a white solid. ESI-LCMS: m/z 776.3 [M+H]⁺; ¹H-NMR (400 MHz, DMSO-d₆): δ 11.40 (d, J=12.2 Hz, 1H), 8.04-7.96 (dd, 1H), 7.43-7.24 (m, 9H), 6.92-6.87 (m, 4H), 5.84 (m, 1H), 4.93 (m, 1H), 4.13 (m, 1H), 3.91-3.39 (m, 17H), 2.82 (t, J=5.6 Hz, 1H), 2.68 (t, J=6.0 Hz, 1H), 1.22-0.97 (m, 12H); ³¹P-NMR (162 MHz, DMSO-d₆): δ 165.06, 157.59.

### Example 22. Synthesis of 5′ End Cap Monomer

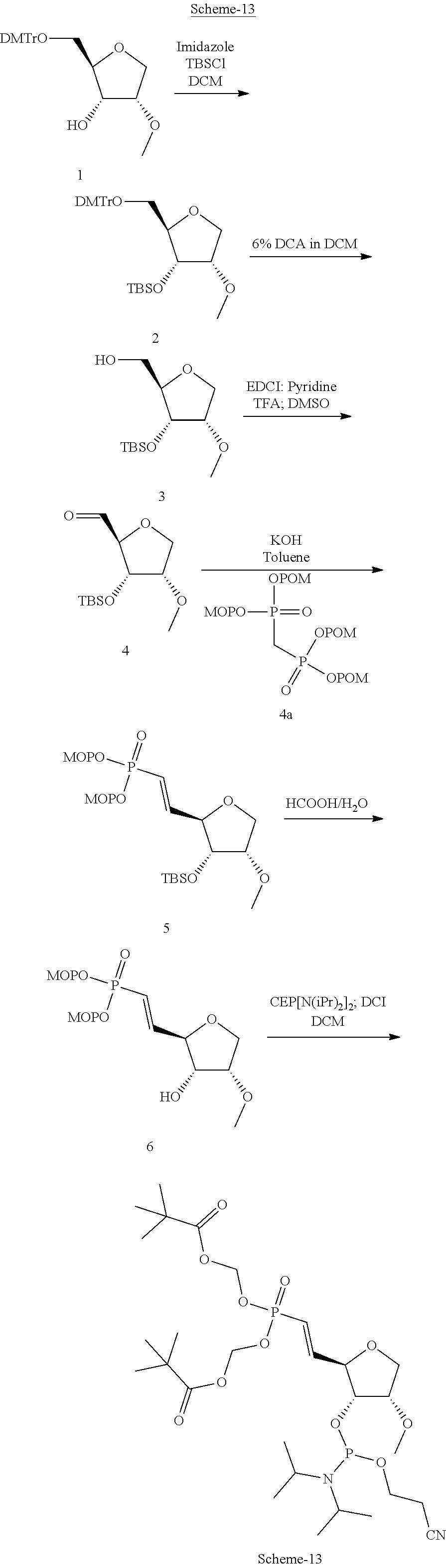

Preparation of (2): To a solution of 1 (11.2 g, 24.7 mmol) in DCM (120 mL), imidazole (4.2 g, 61.9 mmol) and TBSCl (5.6 g, 37.1 mmol) were added at r.t., mixture was stirred at r.t. for 15 hrs, LCMS showed 1 was consumed completely. Mixture was added water (500 mL) and extracted with DCM (50 mL*2). The organic phase was dried over Na₂SO₄ and concentrated to give 2 (16.0 g) as an oil for the next step.

Preparation of (3): To a solution of 2 (16.0 g, 28.4 mmol) was added 6% DCA in DCM (160 mL) and triethylsilane (40 mL) at r.t. The reaction mixture was stirred at r.t. for 2 hrs. TLC showed 2 was consumed completely. Water (300 mL) was added, mixture was extracted with DCM (50 mL*4), organic phase was dried by Na₂SO₄, concentrated by reduce pressure to give crude which was purified by column chromatography (SiO2, PE/EA=10:1 to 1:1) to give 3 (4.9 g, 65.9% yield) as an oil. ESI-LCMS: m/z 263 [M+H]⁺; ¹H-NMR (400 MHz, DMSO-d₆) δ 4.84-4.50 (m, 1H), 4.3-4.09 (m, 1H), 3.90-3.80 (m, 1H), 3.75-3.67 (m, 1H), 3.65-3.57 (m, 2H), 3.50-3.44 (m, 1H), 3.37-3.28 (m, 4H), 0.95-0.78 (s, 9H), 0.13-0.03 (s, 6H).

Preparation of (4): To a solution of 3 (3.3 g, 12.6 mmol) in DMSO (33 mL) was added EDCI (7.2 g, 37.7 mmol). The mixture was added pyridine (1.1 g, 13.8 mmol) and TFA (788.6 mg, 6.9 mmol). The reaction mixture was stirred at r.t. for 3 hrs. TLC (PE/EA=4:1) showed 3 was consumed. The mixture was diluted with EA and water was added. The product was extracted with EA. The organic layer was washed with brine and dried over Na₂SO₄ and concentrated to give the crude. This resulted in to give 4 (3.23 g) as an oil for the next step.

Preparation of (5): To a solution of 4 (3.3 g, 12.6 mmol) in toluene (30 mL) was added POM ester 4a (reference for 4a Journal of Medicinal Chemistry, 2018, 61 (3), 734-744) (7.9 g, 12.6 mmol) and KOH (1.3 g, 22.6 mmol) at r.t. The reaction mixture was stirred at 40° C. for 8 hrs. LCMS showed 4 was consumed. The mixture was diluted with water and EA was added. The product was extracted with EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=91/9 Detector, UV 254 nm. This resulted in to give 5 (5.4 g, 9.5 mmol, 75.9% yield) as an oil. ESI-LCMS: m/z 567.2 [M+H]⁺; ¹H-NMR (400 MHz, CDCl₃) δ 6.89-6.77 (m, 1H), 6.07-5.96 (m, 1H), 5.86-5.55 (m, 4H), 4.85-4.73 (m, 1H), 4.36-4.27 (m, 1H), 4.05-3.96 (m, 1H), 3.95-3.85 (m, 1H), 3.73-3.65 (m, 1H), 3.44-3.35 (m, 3H), 1.30-1.25 (s, 18H), 0.94-0.84 (s, 9H), 0.14-0.05 (s, 6H). ³¹P-NMR (162 MHz, CDCl₃) δ 18.30, 15.11.

Preparation of (6): To a solution of 5 (5.4 g, 9.5 mmol) in HCOOH (30 mL)/H₂O (30 mL)=1:1 at r.t. The reaction mixture was stirred at r.t. for 15 hrs. LCMS showed the reaction was consumed. The mixture was diluted with con. NH₄OH till pH=7.5. The product was extracted with EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% HCOOH)=30/70 increasing to CH₃CN/H₂O (0.5% HCOOH)=70/30 within 45 min, the eluted product was collected at CH₃CN/H₂O (0.5% HCOOH)=59/41 Detector, UV 220 nm. This resulted in to give 6 (2.4 g, 5.7 mmol, 59.4% yield) as an oil. ESI-LCMS: m/z 453.2 [M+H]⁺; ¹H-NMR (400 MHz, DMSO-d₆) δ 6.84-6.68 (m, 1H), 6.07-5.90 (m, 1H), 5.64-5.55 (m, 4H), 5.32-5.24 (m, 1H), 4.23-4.15 (m, 1H), 4.00-3.90 (m, 1H), 3.89-3.80 (m, 1H), 3.78-3.69 (m, 2H), 3.37-3.30 (s, 3H), 1.30-1.10 (s, 18H). ³¹P-NMR (162 MHz, DMSO-d₆) δ 18.14.

Preparation of Example 22 monomer: To a solution of 6 (2.1 g, 4.5 mmol) in DCM (21 mL) were added DCI (452.5 mg, 3.8 mmol) and CEP[N(iPr)₂]₂ (1.8 g, 5.9 mmol) at r.t. The reaction mixture was stirred at r.t. for 15 hrs under N₂ atmosphere. LCMS showed 6 was consumed. The mixture was diluted with water. The product was extracted with DCM (30 mL). The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 28 min, the eluted product was collected at CH₃CN/H₂O (0.5% NH₄HCO₃)=80/20 Detector, UV 254 nm. This resulted in to give Example 22 monomer (2.8 g, 4.3 mmol, 95.2% yield) as an oil. ESI-LCMS: m/z 653.2 [M+H]⁺; ¹H-NMR (400 MHz, DMSO-d₆) δ 6.89-6.77 (m, 1H), 6.11-5.96 (m, 1H), 5.65-5.50 (m, 4H), 4.39-4.34 (d, J=20 Hz, 1H), 4.18-3.95 (m, 2H), 3.94-3.48 (s, 6H), 3.40-3.28 (m, 4H), 2.84-2.75 (m, 2H), 1.26-1.98 (s, 30H). ³¹P-NMR (162 MHz, DMSO-d₆) δ 149.018, 148.736, 17.775, 17.508.

### Example 23. Synthesis of 5′ End Cap Monomer

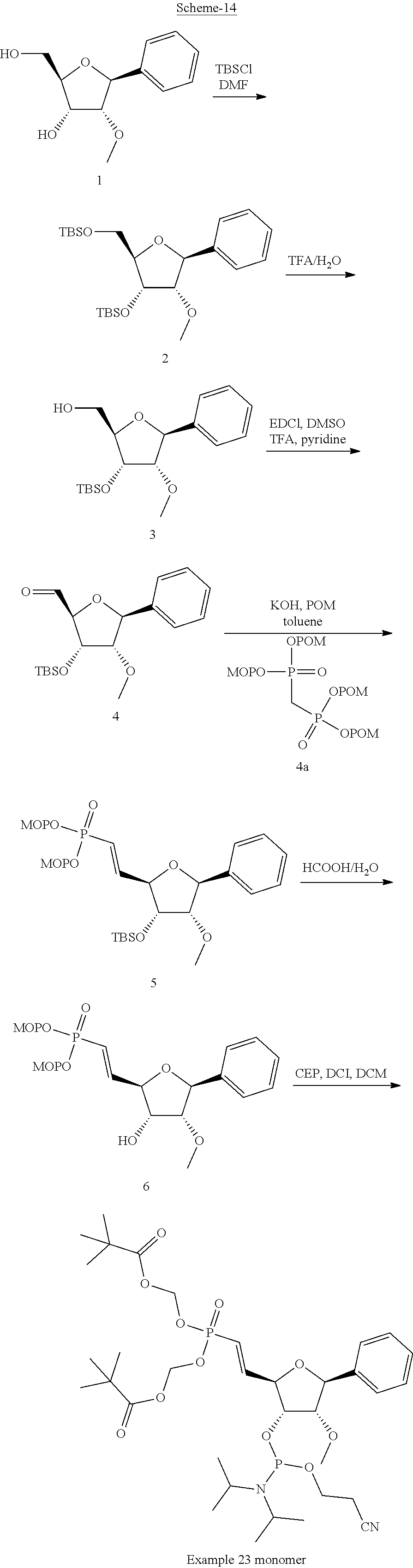

Preparation of (2): To a solution of 1 (ref for 1 Tetrahedron, 2013, 69, 600-606) (10.60 g, 47.32 mmol) in DMF (106 mL), imidazole (11.26 g, 165.59 mmol) and TBSCl (19.88 g, 132.53 mmol) were added. The mixture was stirred at r.t. for 3.5 hrs, LCMS showed 1 was consumed completely. Water was added and extracted with EA, dried over by Na₂SO₄. The filtrate was evaporated under reduced pressure to give 2 (20.80 g, 45.94 mmol, 97.19% yield) for the next step.

Preparation of (3): To a solution of 2 (20.80 g, 45.94 mmol) in THF (248 mL), was added TFA (124 mL) and H₂O (124 mL) at 0° C., reaction mixture was stirred for 30 min. LCMS showed 2 was consumed completely. Then was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 3 (10.00 g, 29.59 mmol, 64.31% yield). ¹H-NMR (400 MHz, DMSO-d₆): δ 7.33-7.18 (m, 5H), 4.83-4.80 (m, 1H), 4.61-4.59 (m, 1H), 4.21-4.19 (m, 1H), 3.75-3.74 (m, 1H), 3.23 (m, 3H), 3.13 (m, 3H), 2.41-2.40 (m, 1H), 0.81 (m, 9H), 0.00 (m, 6H).

Preparation of (4): To a solution of 3 (3.70 g, 10.95 mmol) in DMSO (37 mL) was added EDCI (6.30 g, 32.84 mmol). Then pyridine (0.95 g, 12.05 mmol) and TFA (0.69 g, 6.02 mmol) was added in N₂ atmosphere. The mixture was stirred for 3 hrs at r.t. LCMS showed 3 was consumed completely. Water was poured into and extracted with EA, washed with sat. NaCl (aq.), dried over by Na₂SO₄. The filtrate was evaporated under reduced pressure to give the crude product which was directly used for next step.

Preparation of (5): To a solution of 4 in toluene (100.00 mL), was added 4a (6.93 g, 10.97 mmol) and KOH (1.11 g, 19.78 mmol). It was stirred for 3.5 hrs at 40° C. in N₂ atmosphere. TLC and LCMS showed 4 was consumed completely. Then was extracted with EA, washed with water and sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 5 (4.30 g, 6.70 mmol, 61.17% yield). ¹H-NMR (400 MHz, CDCl₃): δ 7.27-7.26 (m, 4H), 7.17 (m, 1H), 6.94-6.82 (m, 1H), 6.13-6.02 (m, 1H), 5.63-5.56 (m, 4H), 4.90-4.89 (m, 1H), 4.45-4.41 (m, 1H), 3.98-3.95 (m, 1H), 3.39-3.29 (m, 4H), 1.90 (m, 1H), 1.12-0.83 (m, 29H), 0.00 (m, 7H); ³¹P-NMR (162 MHz, CDCl₃): δ 18.021, 14.472.

Preparation of (6): To a solution of 5 (4.30 g, 6.70 mmol) in THF (43.00 mL) was added HCOOH (100 mL) and H₂O (100 mL). It was stirred overnight at r.t. LCMS showed 5 was consumed completely. NH₄OH was poured into it and was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (2.10 g, 3.98 mmol, 59.32% yield). ¹H-NMR (400 MHz, CDCl₃): δ 7.40-7.28 (m, 5H), 7.11-7.00 (m, 1H), 6.19-6.14 (m, 1H), 5.71-5.68 (m, 4H), 4.95-4.94 (m, 1H), 4.48-4.47 (m, 1H), 4.05-4.03 (m, 1H), 3.62-3.61 (m, 1H), 3.46 (m, 3H), 3.00-2.99 (m, 1H), 1.22 (m, 18H); ³¹P-NMR (162 MHz, CDCl₃): δ 18.134.

Preparation of Example 23 monomer: To a solution of 6 (2.10 g, 3.98 mmol) in DCM (21 mL) was added DCI (410 mg, 3.47 mmol). CEP (1.40 g, 4.65 mmol) was added in a N₂ atmosphere. LCMS showed 6 was consumed completely. DCM and H₂O was poured, the organic phase was washed with water and sat. NaCl (aq.), dried over by Na₂SO₄. The filtrate was evaporated under reduced pressure at 40° C. to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 23 monomer (2.10 g, 2.88 mmol). ¹H-NMR (400 MHz, DMSO-d₆): δ 7.39-7.32 (m, 6H), 6.21-6.11 (m, 1H), 5.64-5.61 (m, 4H), 4.91-4.85 (m, 1H), 4.59 (m, 1H), 4.28-4.25 (m, 1H), 3.84-3.60 (m, 5H), 3.36-3.36 (m, 2H), 2.83-2.79 (m, 2H), 1.18-1.14 (m, 29H); ³¹P-NMR (162 MHz, DMSO-d₆): δ 149.588, 148.920, 17.355, 17.010.

### Example 24. Synthesis of 5′ End Cap Monomer

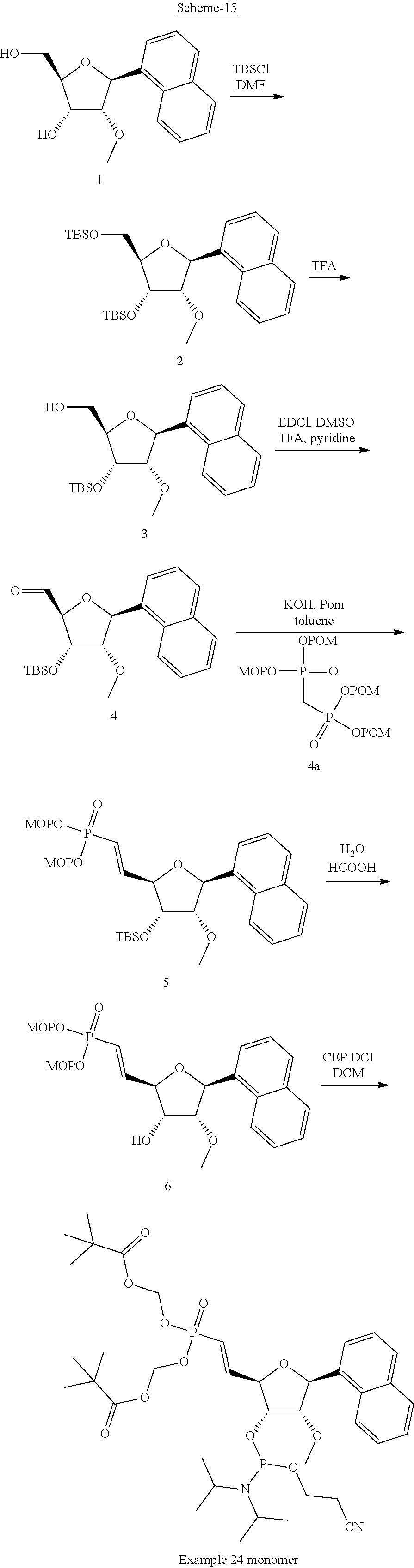

Preparation of (2): To a solution of 1 (5.90 g, 21.50 mmol) in DMF (60.00 mL), imidazole (4.39 g, 64.51 mmol) and TBSCl (7.63 g, 49.56 mmol) were added. The mixture was stirred at r.t. for 3.5 hrs, LCMS showed 1 was consumed completely. Water was added and extracted with EA, dried over by Na₂SO₄. The filtrate was evaporated under reduced pressure to give 2 (11.00 g, 21.91 mmol, 98.19% yield) for the next step. ESI-LCMS: m/z 225.1 [M+H]⁺.

Preparation of (3): To a solution of 2 (11.00 g, 21.91 mmol) in THF (55.00 mL) was added TFA (110.00 mL) and H₂O (55.00 mL) at 0° C., reaction mixture was stirred for 30 min. LCMS showed 2 was consumed completely. Then was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 3 (6.20 g, 16.32 mmol, 72.94% yield). ESI-LCMS: m/z 411.2 [M+H]⁺.

Preparation of (4): To a solution of 3 (3.50 g, 9.02 mmol) in DMSO (35.00 mL) was added EDCI (5.19 g, 27.06 mmol). Then pyridine (0.78 g, 9.92 mmol) and TFA (0.57 g, 4.96 mmol) was added in N₂ atmosphere. The mixture was stirred for 3 h at r.t. Water was poured into it and was extracted with EA, washed with sat. NaCl (aq.), dried over by Na₂SO₄. The filtrate was evaporated under reduced pressure to give the crude product which was directly used for next step. ESI-LCMS: m/z 406.2 [M+H]⁺.

Preparation of (5): To a solution of 4 in toluene (100.00 mL) was added 4a (5.73 g, 9.07 mmol) and KOH (916.3 g, 16.33 mmol). It was stirred for 3.5 h at 40° C. in N₂ atmosphere. Then was extracted with EA, washed with water and sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 5 (5.02 g, 7.25 mmol, 80.44% yield). ESI-LCMS: m/z 693.2 [M+H]⁺; ³¹P-NMR (162 MHz, DMSO-d₆): δ 17.811

Preparation of (6): To a solution of 5 (4.59 g, 6.63 mmol) in THF (46.00 mL) was added HCOOH (92.00 mL) and H₂O (92.00 mL). It was stirred overnight at r.t. NH₄OH was poured into it and extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (2.52 g, 4.36 mmol, 65.80% yield).

Preparation of Example 24 monomer: To a solution of 6 (2.00 g, 3.46 mmol) in DCM (21.00 mL) was added DCI (370.00 mg, 3.11 mmol) and CEP (1.12 g, 4.15 mmol) was added in N₂ atmosphere. DCM and H₂O was poured, the organic phase was washed with water and sat. NaCl (aq.), dried over by Na₂SO₄. The filtrate was evaporated under reduced pressure at 38° C. to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 24 monomer (2.10 g, 2.70 mmol, 78.07% yield). ¹H-NMR (400 MHz, DMSO-d₆): δ 7.39-7.32 (m, 6H), 6.21-6.11 (m, 1H), 5.64-5.61 (m, 4H), 4.91-4.85 (m, 1H), 4.59 (m, 1H), 4.28-4.25 (m, 1H), 3.84-3.60 (m, 5H), 3.36-3.36 (m, 2H), 2.83-2.79 (m, 2H), 1.18-1.14 (m, 29H). ³¹P-NMR (162 MHz, DMSO-d₆): δ 149.588, 148.920, 17.355, 17.010.

### Example 25. Synthesis of Monomer

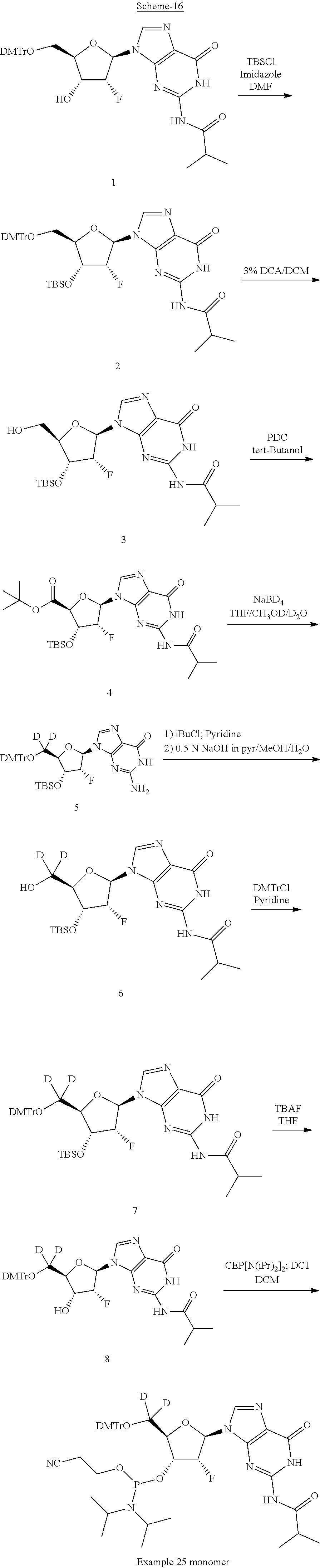

Preparation of (2): To a solution of 1 (35.0 g, 53.2 mmol) in DMF (350 mL) was added imidazole (9.0 g, 133.0 mmol) then added TBSCl (12.0 g, 79.8 mmol) at 0° C. The mixture was stirred at r.t. for 14 hrs. TLC showed 1 was consumed completely. Water was added to the reaction. The product was extracted with EA, The organic layer was washed with NaHCO₃ and brine. Then the solution was concentrated under reduced pressure the crude 2 (41.6 g) as a white solid which was used directly for next step. ESI-LCMS: m/z 772 [M+H]⁺.

Preparation of (3): To a solution of 2 (41.0 g, 53.1 mmol) in 3% DCA (53.1 mmol, 350 mL) and Et₃S¹H (53.1 mmol, 100 mL) at 0° C. The mixture was stirred at 0° C. for 0.5 h. TLC showed 2 was consumed completely. NaHCO₃ was added to the reaction. The product was extracted with EA, The organic layer was washed with NaHCO₃ and brine. Then the solution was concentrated under reduced pressure. The residue silica gel column chromatography (eluent, DCM/MeOH=100:1˜20:1). This resulted in to give 3 (20.0 g, 41.7 mmol, 78.6% over two step) as a white solid. ESI-LCMS: m/z 470 [M+H]⁺; ¹H-NMR (400 MHz, DMSO-d₆): δ 12.12 (s, 1H), 11.67 (s, 1H), 8.28 (s, 1H), 6.12-6.07 (dd, J=15 Hz, 1H), 5.75 (d, J=5 Hz, 1H), 5.48-5.24 (m, 2H), 4.55-4.49 (m, 1H), 3.97 (s, 1H), 3.75-3.55 (m, 2H), 2.79-2.76 (m, 1H), 1.12 (d, J=6 Hz, 6H), 0.88 (s, 9H), 0.11 (d, J=6 Hz, 6H).

Preparation of (4): To the solution of 3 (20 g, 42.6 mmol) in dry DCM (100 mL) and DMF (60 mL) was added PDC (20. g, 85.1 mmol), tert-butyl alcohol (63.1 g, 851.8 mmol) and Ac₂O (43.4 g, 425.9 mmol) at r.t. under N₂ atmosphere. And the reaction mixture was stirred at r.t. for 2 h. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 4 (16.0 g, 29.0 mmol, 68.2% yield) as a white solid. ESI-LCMS: m/z 540 [M+H]⁺; ¹H-NMR (400 MHz, DMSO-d₆): δ 12.12 (s, 1H), 11.69 (s, 1H), 8.28 (s, 1H), 6.21-6.17 (dd, J=15 Hz, 1H), 5.63-5.55 (m, 1H), 4.75-4.72 (m, 1H), 4.41 (d, J=5 Hz, 1H), 2.79-2.76 (m, 1H), 1.46 (s, 9H), 1.13-1.11 (m, 6H), 0.90 (s, 9H), 0.14 (d, J=2 Hz, 6H).

Preparation of (5): To the solution of 4 (16.0 g, 29.6 mmol) in dry THF/MeOD/D₂O=10/2/1 (195 mL) was added NaBD₄ (3.4 g, 88.9 mmol) at r.t. and the reaction mixture was stirred at 50° C. for 2 h. After completion of reaction, adjusted pH value to 7 with CH₃COOD, after addition of water, the resulting mixture was extracted with EA (300 mL). The combined organic layer was washed with water and brine, dried over Na₂SO₄, Then the solution was concentrated under reduced pressure the crude 5 (11.8 g) as a white solid which was used directly for next step. ESI-LCMS: m/z 402 [M+H]⁺.

Preparation of (6): To a solution of 5 (5.0 g, 12.4 mmol) in pyridine (50 mL) was added iBuCl (2.6 g, 24.9 mmol) at 0° C. under N₂ atmosphere. The mixture was stirred at r.t. for 14 h. TLC showed 5 was consumed completely. Then the solution diluted with EA. The organic layer was washed with NaHCO₃ and brine. Then the solution was concentrated under reduced pressure to give the crude. To a solution of the crude in pyridine (50 mL) was added 2N NaOH (MeOH/H₂O=4:1, 15 mL) at 0° C. The mixture was stirred at 0° C. for 10 min. Then the solution diluted with EA. The organic layer was washed with NH₄C1 and brine. Then the solution was concentrated under reduced pressure the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 6 (6 g, 10.86 mmol, 87.17% yield) as a white solid. ESI-LCMS: m/z 472.2 [M+H]⁺; ¹H-NMR (400 MHz, DMSO-d₆): δ 12.12 (s, 1H), 11.67 (s, 1H), 8.28 (s, 1H), 6.12-6.07 (dd, J=15 Hz, 1H), 5.48-5.24 (m, 2H), 5.22 (s, 1H), 4.55-4.49 (m, 1H), 3.97 (d, J=5 Hz, 1H), 2.79-2.76 (m, 1H), 1.12 (d, J=6 Hz, 6H), 0.88 (s, 9H), 0.11 (d, J=6 Hz, 6H).

Preparation of (7): To a solution of 6 (3.8 g, 8.1 mmol) in pyridine (40 mL) was added DMTrCl (4.1 g, 12.1 mmol) at 20° C. The mixture was stirred at 20° C. for 1 h. TLC showed 7 was consumed completely. Water was added to the reaction. The product was extracted with EA, The organic layer was washed with NaHCO₃ and brine. Then the solution was concentrated under reduced pressure to give the crude product of 7 (6 g, 7.6 mmol, 94.3% yield) as a yellow solid. ESI-LCMS: m/z 775 [M+H]⁺.

Preparation of (8): To a solution of 7 (6.0 g, 7.75 mmol) in THF (60 mL) was added TBAF (2.4 g, 9.3 mmol). The mixture was stirred at r.t. for 1 h. TLC showed 7 was consumed completely. Water was added to the reaction. The product was extracted with EA, The organic layer was washed with NaHCO₃ and brine. Then the solution was concentrated under reduced pressure, the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in to give 8 (4.0 g, 5.9 mmol, 76.6% yield) as a white solid. ESI-LCMS: m/z 660 [M+H]⁺; ¹H-NMR (400 MHz, DMSO-d₆): δ 12.12 (s, 1H), 11.67 (s, 1H), 8.12 (s, 1H), 7.34-7.17 (m, 9H), 6.83-6.78 (m, 4H), 6.23-6.18 (m, 1H), 5.66 (d, J=7 Hz, 1H), 5.48-5.35 (m, 1H), 4.65-4.54 (m, 1H), 3.72 (d, J=2 Hz, 6H), 2.79-2.73 (m, 1H), 1.19-1.06 (m, 6H).

Preparation of Example 25 monomer: To a solution of 9 (4.0 g, 6.1 mmol) in DCM (40 mL) was added DCI (608 mg, 5.1 mmol) and CEP (2.2 g, 7.3 mmol) under N₂ pro. The mixture was stirred at 20° C. for 0.5 h. TLC showed 9 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give Example 25 monomer (5.1 g, 5.81 mmol, 95.8% yield) as a white solid. ESI-LCMS: m/z 860 [M+H]⁺; ¹H-NMR (400 MHz, DMSO-d₆): δ 12.12 (s, 1H), 11.67 (s, 1H), 8.12 (s, 1H), 7.34-7.17 (m, 9H), 6.83-6.78 (m, 4H), 6.23-6.18 (m, 1H), 5.67-5.54 (m, 1H), 4.70-4.67 (m, 1H), 4.23-4.20 (m, 1H), 3.72 (m, 6H), 3.60-3.48 (m, 3H), 2.79-2.58 (m, 3H), 1.13-0.94 (m, 18H); ³¹P-NMR (162 MHz, DMSO-d₆): δ 150.31, 150.26, 140.62, 149.57.

### Example 26: Synthesis of Monomer

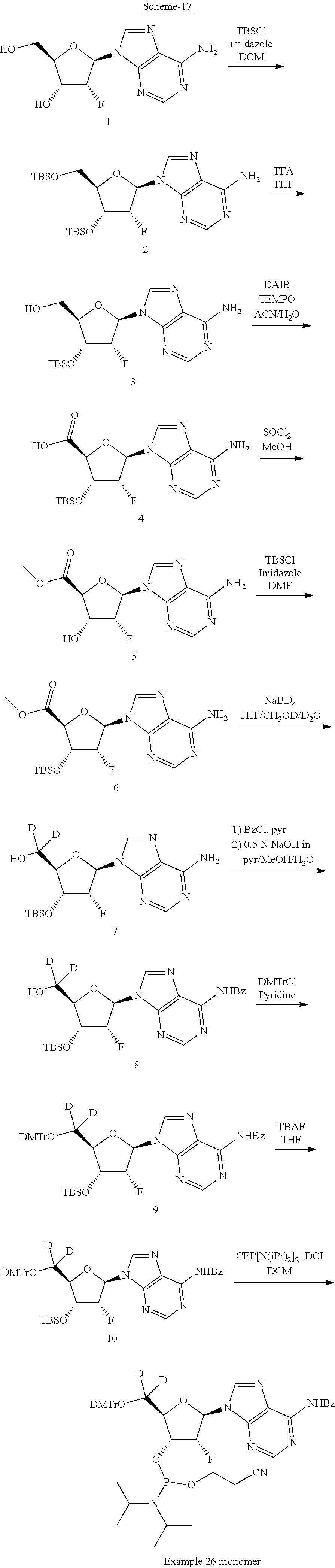

Preparation of (2): To a solution of 1 (35 g, 130.2 mmol) in DMF (350 mL) was added imidazole (26.5 g, 390.0 mmol) then added TBSCl (48.7 g, 325.8 mmol) at 0° C. The mixture was stirred at r.t. for 14 h. TLC showed 1 was consumed completely. Water was added to the reaction. The product was extracted with EA, The organic layer was washed with NaHCO₃ and brine. Then the solution was concentrated under reduced pressure the crude 2 (64.6 g) as a white solid which was used directly for next step. ESI-LCMS: m/z 498 [M+H]⁺.

Preparation of (3): To a solution of 2 (64.6 g, 130.2 mmol) in THF (300 mL) and added TFA/H₂O (1:1, 300 mL) at 0° C. The mixture was stirred at 0° C. for 2 h. TLC showed 2 was consumed completely. NaHCO₃ was added to the reaction. The product was extracted with EA, The organic layer was washed with NaHCO₃ and brine. Then the solution was concentrated under reduced pressure. The residue was purified by silica gel column chromatography (eluent, DCM:MEOH=100:1˜20:1). This resulted in to give 3 (31.3 g, 81.7 mmol, 62.6% over two step) as a white solid. ESI-LCMS: m/z 384 [M+H]⁺.

Preparation of (4): To a solution of 3 (31.3 g, 81.7 mmol) in ACN/H₂O (1:1, 350 mL) was added DAIB (78.0 g, 244.0 mmol) and Tempo (3.8 g, 24.4 mmol). The mixture was stirred at 40° C. for 2 h. TLC showed 3 was consumed completely. Then filtered to give 4 (22.5 g, 55.5 mmol, 70.9%) as a white solid. ESI-LCMS: m/z 398 [M+H]⁺.

Preparation of (5): To a solution of 4 (22.5 g, 55.5 mmol) in MeOH (225 mL) held at −15° C. with an ice/MeOH bath was added SOCl₂ (7.6 mL, 94.5 mmol), dropwise at such a rate that the reaction temp did not exceed 7° C. After the addition was complete, cooling was removed, the reaction was allowed to stir at room temp. The mixture was stirred at r.t. for 14 h. TLC showed 4 was consumed completely. Then the solution was concentrated under reduced pressure to get crude 5 (23.0 g) as a white solid which was used directly for next step. ESI-LCMS: m/z 298 [M+H]⁺.

Preparation of (6): To a solution of 5 (23 g, 55.5 mmol) in DMF (220 mL) was added imidazole (11.6 g, 165.0 mmol) then added TBSCl (12.3 g, 82.3 mmol) at 0° C. The mixture was stirred at 20° C. for 14 h. TLC showed 1 was consumed completely. Water was added to the reaction. The product was extracted with EA, The organic layer was washed with NaHCO₃ and brine. Then the solution was concentrated under reduced pressure. The residue was purified by silica gel column chromatography (eluent, DCM:MEOH=100:1˜20:1). This resulted in to give 6 (21.3 g, 51.1 mmol, 90% over two step) as a white solid. ESI-LCMS: m/z 412 [M+H]⁺.

Preparation of (7): To the solution of 6 (21.0 g, 51.0 mmol) in dry THF/MeOD/D₂O=10/2/1 (260.5 mL) was added NaBD₄ (6.4 g, 153.1 mmol) at r.t. and the reaction mixture was stirred at 50° C. for 2 h. After completion of reaction, the resulting mixture was added CH₃COOD to pH=7, after addition of water, the resulting mixture was extracted with EA (300 mL). The combined organic layer was washed with water and brine, dried over Na₂SO₄. Then the solution was concentrated under reduced pressure and the residue was used for next step without further purification. ESI-LCMS: m/z 386 [M+H]⁺.

Preparation of (8): To a stirred solution of 7 (14.0 g, 35 mmol) in pyridine (50 mL) were added BzCl (17.2 g, 122.5 mmol) at 0° C. under N₂ atmosphere. The mixture was stirred at r.t. for 14 h. TLC showed 7 was consumed completely. Then the solution diluted with EA. The organic layer was washed with NaHCO₃ and brine. Then the solution was concentrated under reduced pressure and the residue was used for next step without further purification. To a solution of the crude in pyridine (300 mL) then added 2M NaOH (MeOH:H₂O=4:1, 60 mL) at 0° C. The mixture was stirred at 0° C. for 10 min. Then the solution diluted with EA. The organic layer was washed with NH₄C1 and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 8 (14 g, 28.02 mmol, 69.21% yield) as a white solid. ESI-LCMS: m/z 490 [M+H]⁺; ¹H-NMR (400 MHz, DMSO-d₆): δ 11.24 (s, 1H), 8.76 (s, 1H), 8.71 (m, 1H), 8.04 (d, J=7 Hz, 2H), 7.66-7.10 (m, 5H), 6.40-6.35 (dd, 1H), 5.71-5.56 (m, 1H), 5.16 (s, 1H), 4.79-4.72 (m, 1H), 4.01 (m, 1H), 0.91 (s, 9H), 0.14 (m, 6H).

Preparation of (9): To a solution of 8 (5.1 g, 10.4 mmol) in pyridine (50 mL) was added DMTrCl (5.3 g, 15.6 mmol). The mixture was stirred at r.t. for 1 h. TLC showed 8 was consumed completely. Water was added to the reaction. The product was extracted with EA, The organic layer was washed with NaHCO₃ and brine. Then the solution was concentrated under reduced pressure and the residue was used for next step without further purification. ESI-LCMS: m/z 792 [M+H]⁺.

Preparation of (10): To a solution of 9 (7.9 g, 10.0 mmol) in THF (80 mL) was added 1M TBAF in THF (12 mL). The mixture was stirred at r.t. for 1 h. TLC showed 9 was consumed completely. Water was added to the reaction. The product was extracted with EA, The organic layer was washed with NaHCO₃ and brine. Then the solution was concentrated under reduced pressure the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in to give 10 as a white solid. ESI-LCMS: m/z 678 [M+H]⁺; ¹H-NMR (400 MHz, DMSO-d₆): δ 11.25 (s, 1H), 8.74 (s, 1H), 8.62 (s, 1H), 8.04 (d, J=7 Hz, 2H), 7.66-7.53 (m, 3H), 7.33-7.15 (m, 9H), 6.82-6.78 (m, 4H), 6.43 (d, J=20 Hz, 1H), 5.76-5.60 (m, 1H), 4.88-4.80 (m, 1H), 4.13 (d, J=8 Hz, 1H), 3.71 (m, 6H).

Preparation of Example 26 monomer: To a solution of 10 (6.2 g, 9.1 mmol) in DCM (60 mL) was added DCI (1.1 g, 9.4 mmol) and CEP (3.3 g, 10.9 mmol) under N₂ pro. The mixture was stirred at 20° C. for 0.5 h. TLC showed 10 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 26 monomer (7.5 g, 8.3 mmol, 90.7%) as a white solid. ESI-LCMS: m/z 878 [M+H]⁺; ¹H-NMR (400 MHz, DMSO-d₆): δ 11.25 (s, 1H), 8.68-8.65 (dd, 2H), 8.04 (m, 2H), 7.66-7.53 (m, 3H), 7.33-7.15 (m, 9H), 6.82-6.78 (m, 4H), 6.53-6.43 (m, 1H), 5.96-5.81 (m, 1H), 5.36-5.15 (m, 1H), 4.21 (m, 1H), 3.86-3.52 (m, 1OH), 2.79-2.61 (m, 2H), 1.21-0.99 (m, 12H); ³¹P-NMR (162 MHz, DMSO-d₆): δ 149.60, 149.56, 149.48.

### Example 27. Synthesis of End Cap Monomer

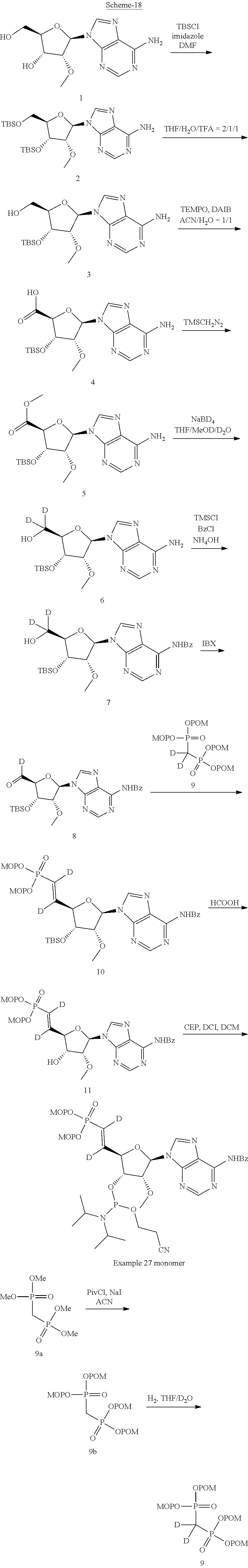

Preparation of (2): To a solution of 1 (20.0 g, 71.2 mmol) in dry pyridine (200.0 mL) was added TBSCl (26.8 g, 177.9 mmol) and imidazole (15.6 g, 227.8 mmol). The mixture was stirred at r.t. for 15 h. TLC showed 1 was consumed completely. The reaction mixture was concentrated to give residue. The residue was quenched with DCM (300.0 mL). The DCM layer was washed with H₂O (100.0 mL*2) and brine. The DCM layer concentrated to give crude 2 (45.8 g) as a yellow oil. The crude used to next step directly. ESI-LCMS m/z 510.5 [M+H]⁺.

Preparation of (3): To a mixture solution of 2 (45.8 g) in THF (300.0 mL) was added mixture of H₂O (100.0 mL) and TFA (100.0 mL) at 0° C. over 30 min. Then the reaction mixture was stirred at 0° C. for 4 h. TLC showed the 2 was consumed completely. The reaction mixture pH was adjusted to 7-8 with NH₃·H₂O (100 mL). Then the mixture was extracted with EA (500.0 mL*2). The combined EA layer was washed with brine and concentrated to give crude which was purified by c.c. (PE:EA=5:1˜1:0) to give compound 3 (21.0 g, 53.2 mmol, 74.7% yield over 2 steps) as a white solid. ESI-LCMS m/z 396.2 [M+H]⁺.

Preparation of (4): To a solution of 3 (21.0 g, 53.2 mmol) in ACN (100.0 mL) and water (100.0 mL) were added (diacetoxyiodo)benzene (51.0 g, 159.5 mmol) and TEMPO (2.5 g, 15.9 mmol), The reaction mixture was stirred at 40° C. for 1 h. TLC showed the 3 was consumed completely. The reaction mixture was cooled down to r.t. and filtered, the filtrate was concentrated to give crude which was purified by crystallization (ACN) to give 4 (14.5 g, 35.4 mmol, 66.2% yield). ESI-LCMS m/z 410.1[M+H]⁺.

Preparation of (5): To a solution of 4 (14.5 g, 35.4 mmol) in toluene (90.0 mL) and MeOH (60.0 mL) was added trimethylsilyldiazomethane (62.5 mL, 2.0 M, 141.8 mmol) at 0° C., then stirred at r.t. for 2 h. TLC showed the 4 was consumed completely. The solvent was removed under reduce pressure, the residue was purified by crystallization (ACN) to give 5 (10.0 g, 23.6 mmol, 66.6% yield). ESI-LCMS m/z 424.2 [M+H]+

Preparation of (6): To the solution of 5 (10.0 g, 23.6 mmol) in dry THF/MeOD/D₂O=10/2/1 (100.0 mL) was added NaBD₄ (2.98 g, 70.9 mmol) three times during an hour at 40° C., the reaction mixture was stirred at r.t. for 2.0 h. The resulting mixture was added CH₃COOD change pH=7.5, after addition of water, the resulting mixture was extracted with EA (50.0 mL*3). The combined organic layer was washed with water and brine, dried over Na₂SO₄, concentrated to give a residue which was purified by c.c. (PE/EA=1:1˜1:0). This resulted in to give 6 (6.1 g, 15.4 mmol, 65.3% yield) as a white solid. ESI-LCMS m/z 398.1 [M+H]⁺; ¹H-NMR (400 MHz, DMSO-d₆) δ 8.28 (s, 1H), 8.02 (s, 1H), 7.23 (s, 2H), 5.86 (d, J=6.4 Hz, 1H), 5.26 (s, 1H), 4.42-4.41 (m, 1H), 4.35-4.32 (m, 1H), 3.82 (d, J=2.6 Hz, 1H), 3.14 (s, 3H), 0.78 (s, 9H), 0.00 (d, J=0.9 Hz, 6H).

Preparation of (7): To a solution of 6 (6.1 g, 15.4 mmol) in pyridine (60.0 mL) was added the benzoyl chloride (6.5 g, 46.2 mmol) drop wise at 5° C. The reaction mixture was stirred at r.t. for 2 h. TLC showed the 6 was consumed completely. The reaction mixture was cooled down to 10° C. and quenched with H₂O (20.0 mL), extracted with EA (200.0 mL*2), combined the EA layer. The organic phase was washed with brine and dried over Na₂SO₄, concentrated to give the crude (12.0 g) which was dissolved in pyridine (60.0 mL), cooled to 0° C., 20.0 mL NaOH (2 M in methanol:H₂O=4:1) was added and stirred for 10 min. The reaction was quenched by saturated solution of ammonium chloride, the aqueous layer was extracted with EA (200.0 mL*2), combined the EA layer, washed with brine and dried over Na₂SO₄, concentrated. The residue was purified by c.c. (PE/EA=10:1˜1:1) to give 7 (7.0 g, 13.9 mmol, 90.2% yield). ESI-LCMS m/z 502.2 [M+H]⁺; ¹H-NMR (400 MHz, DMSO-d₆) δ 11.24 (s, 1H, exchanged with D₂O) 8.77 (s, 2H), 8.04-8.06 (m, 2H), 7.64-7.66 (m, 2H), 7.54-7.58 (m, 2H), 6.14-6.16 (d, J=5.9 Hz, 1H), 5.20-5.23 (m, 1H), 4.58-4.60 (m, 1H), 4.52-4.55 (m, 1H), 3.99-4.01 (m, 1H), 3.34 (s, 4H), 0.93 (s, 9H), 0.14-0.15 (d, J=1.44 Hz, 6H).

Preparation of (8): To a stirred solution of 7 (5.5 g, 10.9 mmol) in DMSO (55.0 mL) was added EDCI (6.3 g, 32.9 mmol), pyridine (0.9 g, 10.9 mmol) and TFA (0.6 g, 5.5 mmol), the reaction mixture was stirred at r.t. for 15 h. The reaction was quenched with water and extracted with EA (100.0 mL). The organic phase was washed by brine, dried over Na₂SO₄, The organic phase was evaporated to dryness under reduced pressure to give a residue 8 (4.8 g) which was used directly to next step. ESI-LCMS: m/z 517.1 [M+H₂O]⁺.

Preparation of (9b): A solution of 9a (35.0 g, 150.8 mmol) and NaI (90.5 g, 603.4 mmol) in dry ACN (180.0 mL) was added chloromethyl pivalate (113.6 g, 754.3 mmol) at r.t., the reaction was stirred at 80° C. for 4 h. The reaction was cooled to r.t. and quenched by water, then the mixture was extracted with EA (500.0 mL*3), combined the organic layer was washed with saturated solution of ammonium chloride, followed by with brine and dried over Na₂SO₄. Then the organic layer was concentrated to give a residue which was purified by c.c., this resulted in to give 9b (38.0 g, 60.1 mmol, 39.8% yield) as a white solid. ESI-LCMS m/z 655.2 [M+Na]⁺; ¹H-NMR (400 MHz, CDCl₃): δ 5.74-5.67 (m, 8H), 2.67 (t, J=21.6 Hz, 2H), 1.23 (s, 36H).

Preparation of (9): 3.8 g 10% Pd/C was washed with dry THF (30.0 mL) three times. Then transferred into a round-bottom flask charged with 9b (38.0 g, 60.1 mmol) and solvent (dry THF:D₂O=5:1, 400.0 mL), the mixture was stirred at 80° C. under 1 L H₂ balloon for 15 h. The reaction was cooled to r.t. and extracted with EA (500.0 mL*3), combined the organic layer was washed with brine and dried over Na₂SO₄. The residue 9 (3.0 g, 3.7 mmol, 38.8% yield) as a white solid was used directly to next step without further purification. ESI-LCMS m/z 657.2 [M+Na]⁺; ¹H-NMR (400 MHz, CDCl₃): δ 5.74-5.67 (m, 8H), 1.23 (s, 36H).

Preparation of (10): A solution of 8 (4.8 g, 9.6 mmol), 9 (7.3 g, 11.5 mmol) and K2CO3 (4.0 g, 38.8 mmol) in dry THF (60.0 mL) and D₂O (20.0 mL) was stirred at r.t. 18 h. LC-MS showed 8 was consumed completely. The product was extracted with EA (300.0 mL) and the organic layer was washed with brine and dried over Na₂SO₄. Then the organic layer was concentrated to give a residue which was purified by c.c. (PE/EA=5:1˜1:1) and MPLC. This resulted in to give 10 (3.0 g, 3.7 mmol, 38.8% yield) as a white solid. ESI-LCMS m/z 806.4[M+H]⁺; ¹H-NMR (400 MHz, DMSO-d₆): δ 11.25 (s, 1H, exchanged with D₂O) 8.75 (s, 2H), 8.07-8.05 (d, J=8.0 Hz, 2H), 7.67-7.54 (m, 3H), 6.05 (d, J=5.1 Hz, 1H), 5.65-5.58 (m, 4H), 4.80-4.70 (m, 2H), 4.59-4.57 (m, 1H), 3.36 (s, 3H), 1.11 (s, 9H), 1.10 (s, 9H), 0.94 (s, 9H), 0.17-0.16 (m, 6H); ³¹P NMR (162 MHz, DMSO-d₆) δ 17.02.

Preparation of (11): To a round-bottom flask was added 10 (3.0 g, 3.7 mmol) in a mixture of H₂O (30.0 mL), HCOOH (30.0 mL). The reaction mixture was stirred at 40° C. for 15 hrs. LC-MS showed the 10 was consumed completely. The reaction mixture was adjusted the pH=6-7 with con. NH₃·H₂O (100.0 mL). Then the mixture was extracted with DCM (100.0 mL*3). The combined DCM layer was dried over Na₂SO₄. Filtered and filtrate was concentrated to give crude which was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 20 min, the eluted product was collected at CH₃CN/H₂O (0.5% NH₄HCO₃)=3/2; Detector, UV 254 nm. To give product 11 (1.8 g, 2.6 mmol, 70.3% yield). ESI-LCMS m/z=692.2[M+H]⁺; ¹H-NMR (400 MHz, DMSO-d₆): δ 11.11 (s, 1H, exchanged with D₂O) 8.71-8.75 (d, J=14.4, 2H), 8.04-8.06 (m, 2H), 7.64-7.65 (m, 1H), 7.54-7.58 (m, 2H), 6.20-6.22 (d, J=5.4, 2H), 5.74-5.75 (d, J=5.72, 2H), 5.56-5.64 (m, 4H), 4.64-4.67 (m, 1H), 4.58-4.59 (m, 1H), 4.49-4.52 (m, 1H), 3.37 (s, 3H), 1.09-1.10 (d, J=1.96, 18H); ³¹P NMR (162 MHz, DMSO-d₆) δ 17.46.

Preparation of Example 27 monomer: To a solution of 11 (1.8 g, 2.6 mmol) in DCM (18.0 mL) was added the DCI (276.0 mg, 2.3 mmol), then CEP[N(ipr)₂]₂ (939.5 mg, 3.1 mmol) was added. The mixture was stirred at r.t. for 1 h. TLC showed 11 consumed completely. The reaction mixture was washed with H₂O (50.0 mL*2) and brine (50.0 mL*2), dried over Na₂SO₄ and concentrated to give crude which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. The product was concentrated to give Example 27 monomer (2.0 g, 2.2 mmol, 86.2% yield) as a white solid. ESI-LCMS m/z 892.3[M+H]⁺; ¹H-NMR (400 MHz, DMSO-d₆): δ 11.27 (s, 1H, exchanged with D₂O) 8.72-8.75 (m, 2H), 8.04-8.06 (m, 2H), 7.54-7.68 (m, 3H), 6.20-6.26 (m, 1H), 5.57-5.64 (m, 4H), 4.70-4.87 (m, 3H), 3.66-3.88 (m, 4H), 3.37-3.41 (m, 3H), 2.82-2.86 (m, 2H), 1.20-1.21 (m, 12H), 1.08-1.09 (m, 18H); ³¹P-NMR (162 MHz, DMSO-d₆): δ 150.03, 149.19, 17.05, 16.81.

### Example 28. Synthesis of 5′ End Cap Monomer

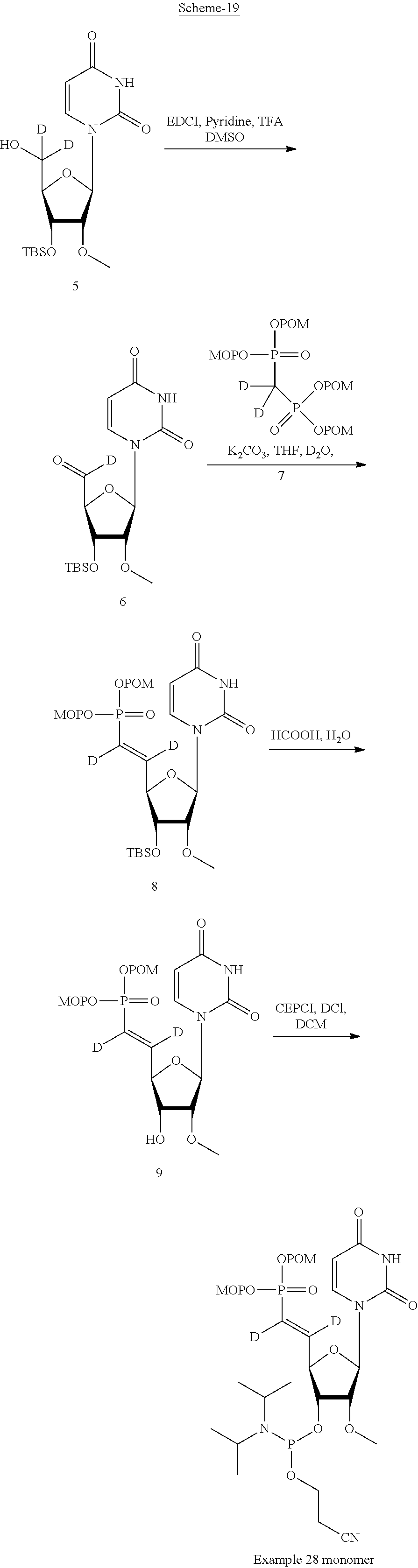

Preparation of (6): To a stirred solution of 5 (8.0 g, 21.3 mmol, Scheme 3) in DMSO (80.0 mL) were added EDCI (12.2 g, 63.9 mmol), pyridine (1.7 g, 21.3 mmol), TFA (1.2 g, 10.6 mmol) at r.t. And the reaction mixture was stirred at r.t. for 1.5 h. The reaction was quenched with water and extracted with EA (200.0 mL). The organic phase was washed by brine, dried over Na₂SO₄, The organic phase was evaporated to dryness under reduced pressure to give a residue 6 which was used directly to next step. ESI-LCMS: m/z 372.3 [M+H]⁺.

Preparation of (8): To a solution of K2CO3 (5.5 g, 8.3 mmol) in dry THF (60.0 mL) and D₂O (20.0 mL) was added a solution of 6 (8.0 g, 21.5 mmol) in dry THF (10.0 mL). The reaction mixture was stirred at r.t. overnight. LC-MS showed 6 was consumed completely. The product was extracted with EA (300.0 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 8 (5.0 g, 7.3 mmol, 40.0%) as a white solid. ESI-LCMS: m/z 679.3 [M+H]⁺; ¹H-NMR (400 MHz, Chloroform-d): δ 9.91 (s, 1H), 7.29 (d, J=8.1 Hz, 1H), 5.82 (d, J=2.7 Hz, 1H), 5.72 (d, J=8.1 Hz, 1H), 5.65-5.54 (m, 4H), 4.43 (dd, J=7.2, 3.2 Hz, 1H), 3.92 (dd, J=7.2, 5.0 Hz, 1H), 3.65 (dd, J=5.1, 2.7 Hz, 1H), 3.44 (s, 3H), 1.13 (s, 18H), 0.82 (s, 9H), 0.01 (d, J=4.8 Hz, 6H); ³¹P NMR (162 MHz, Chloroform-d): δ 16.40.

Preparation of (9): To a solution of HCOOH (50.0 mL) and H₂O (50.0 mL) was added 8 (5.0 g, 7.3 mmol). The reaction mixture was stirred at 40° C. overnight. LC-MS showed 8 was consumed completely. A solution of NaHCO₃ (500.0 mL) was added. The product was extracted with EA (300.0 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 9 (3.0 g, 5.4 mmol, 73.2%) as a white solid. ESI-LCMS: m/z 565.2 [M+H]⁺; ¹H-NMR (400 MHz, DMSO-d₆): δ 11.43 (s, 1H), 7.64 (d, J=8.1 Hz, 1H), 5.83 (d, J=4.3 Hz, 1H), 5.69-5.56 (m, 5H), 5.54 (d, J=6.7 Hz, 1H), 4.37 (dd, J=6.1, 2.9 Hz, 1H), 4.12 (q, J=6.1 Hz, 1H), 3.96 (dd, J=5.4, 4.3 Hz, 1H), 3.39 (s, 3H), 1.16 (s, 18H); ³¹P NMR (162 MHz, DMSO-d₆): δ 17.16.

Preparation of Example 28 monomer: To a suspension of 9 (2.6 g, 4.6 mmol) in DCM (40.0 mL) was added DCI (0.5 g, 5.6 mmol) and CEP[N(iPr)₂]₂ (1.7 g, 5.6 mmol). The mixture was stirred at r.t. for 1.0 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 28 monomer (3.0 g, 3.9 mmol, ⁸5.2%) as a white solid. ESI-LCMS: m/z 765.3 [M+H]⁺; ¹H-NMR (400 MHz, DMSO-d₆): δ 11.44 (s, 1H), 7.71 (dd, J=8.1, 3.8 Hz, 1H), 5.81 (dd, J=4.4, 2.5 Hz, 1H), 5.74-5.53 (m, 5H), 4.59-4.33 (m, 2H), 4.20-4.14 (m, 1H), 3.88-3.53 (m, 4H), 3.39 (d, J=16.2 Hz, 3H), 2.80 (td, J=5.9, 2.9 Hz, 2H), 1.16 (d, J=1.9 Hz, 30H); ³¹P-NMR (162 MHz, DMSO-d₆): δ 147.68, 149.16, 16.84, 16.55.

### Example 29. Synthesis of Monomer

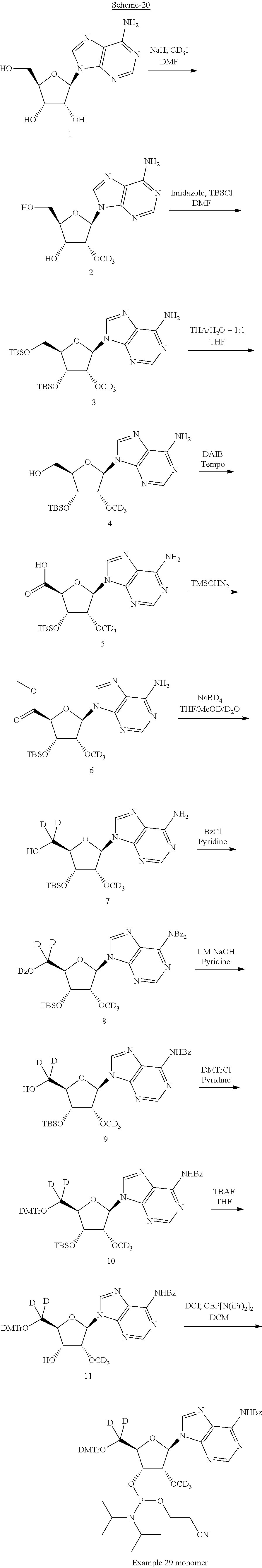

Preparation of (2): To a solution of 1 (26.7 g*2, 0.1 mol) in DMF (400 mL) was added sodium hydride (4.8 g, 0.1 mol) for 30 min, then was added CD₃I (16 g, 0.1 mol) at 0° C. for 2.5 hr (ref. for selective 2′-O-alkylation reaction conditions, J. Org. Chem. 1991, 56, 5846-5859). The mixture was stirring at r.t. for another 1 h. LCMS showed the reaction was consumed. The mixture was filtered and the clear solution was evaporated to dryness and was evaporated with CH₃OH. The crude was purified by silica gel column (SiO2, DCM/MeOH=50:1˜15:1). This resulted in to give the product 2 (35.5 g, 124.6 mmol, 62% yield) as a solid. ESI-LCMS: m/z 285 [M+H]⁺.

Preparation of (3): To a solution of 2 (35.5 g, 124.6 mmol) in pyridine (360 mL) was added imidazole (29.7 g, 436.1 mmol) and TBSCl (46.9 g, 311.5 mmol). The mixture was stirred at r.t. over night. LCMS showed 2 was consumed completely. The reaction was quenched with water (500 mL). The product was extracted into ethyl acetate (1 L). The organic layer was washed with brine and dried over anhydrous Na₂SO₄. The crude was purified by silica gel column (SiO2, PE/EA=4:1˜1:1). This resulted in to give the product 3 (20.3 g, 39.6 mmol, 31.8% yield) as a solid. ESI-LCMS: m/z 513 [M+H]⁺; ¹H-NMR (400 MHz, DMSO-d₆): δ 8.32 (m, 1H), 8.13 (m, 1H), 7.31 (m, 2H), 6.02-6.01 (d, J=4.0 Hz, 1H), 4.60-4.58 (m, 1H), 4.49-4.47 (m, 1H), 3.96-3.86 (m, 2H), 3.72-3.68 (m, 1H), 0.91-0.85 (m, 18H), 0.13-0.01 (m, 12H).

Preparation of (4): To a solution of 3 (20.3 g, 39.6 mmol) in THF (80 mL) was added TFA (20 mL) and water (20 mL) at 0° C. The reaction mixture was stirred at 0° C. for 5 h. LC-MS showed 3 was consumed completely. Con. NH₄OH was added to the mixture at 0° C. to quench the reaction until the pH=7.5. The product was extracted into ethyl acetate (200 mL). The organic layer was washed with brine and dried over anhydrous Na₂SO₄. The solution was then concentrated under reduced pressure and the residue was washed by PE/EA=5:1. This resulted in to give 4 (10.5 g, 26.4 mmol, 66.6% yield) as a white solid. ESI-LCMS: m/z 399 [M+H]⁺; ¹H-NMR (400 MHz, DMSO-d₆): δ 8.41 (m, 1H), 8.14 (m, 1H), 7.37 (m, 2H), 5.99-5.97 (d, J=8.0 Hz, 1H), 5.43 (m, 1H), 4.54-4.44 (m, 2H), 3.97-3.94 (m, 1H), 3.70-3.53 (m, 2H), 0.91 (m, 9H), 0.13-0.12 (m, 6H).

Preparation of (5): To a solution of 4 (10.5 g, 26.4 mmol) in ACN/H₂O=1:1 (100 mL) was added DAIB (25.4 g, 79.2 mmol) and TEMPO (1.7 g, 7.9 mmol). The reaction mixture was stirred at 40° C. for 2 h. LCMS showed 4 was consumed. The mixture was diluted with EA and water was added. The product was extracted with EA. The organic layer was washed with brine and dried over anhydrous Na₂SO₄. The solution was then concentrated under reduced pressure and the residue was washed by ACN. This resulted in to give 5 (6.3 g, 15.3 mmol, 57.9% yield) as a white solid. ESI-LCMS: m/z 413 [M+H]⁺; ¹H-NMR (400 MHz, DMSO-d₆): δ=8.48 (m, 1H), 8.16 (m, 1H), 7.41 (m, 2H), 6.12-6.10 (d, J=8.0 Hz, 1H), 4.75-4.73 (m, 1H), 4.42-4.36 (m, 2H), 3.17 (m, 6H), 2.07 (m, 2H), 0.93 (m, 9H), 0.17-0.15 (m, 6H).

Preparation of (6): To a solution of 5 (6.3 g, 15.3 mmol) in toluene (36 mL) and methanol (24 mL) was added (trimethylsilyl)diazomethane (7.0 g, 61.2 mmol) till the yellow color not disappear at r.t. for 2 min. LCMS showed the reaction was consumed. The solvent was removed to give the cured 6 (6.0 g) as a solid which used for the next step. ESI-LCMS: m/z 427 [M+H]⁺; ¹H-NMR (400 MHz, DMSO-d₆): δ 8.45 (m, 1H), 8.15 (m, 1H), 7.35 (m, 2H), 6.12-6.10 (d, J=8.0 Hz, 1H), 4.83-4.81 (m, 1H), 4.50-4.46 (m, 1H), 3.73 (m, 3H), 3.31 (m, 1H), 0.93 (m, 9H), 0.15-0.14 (m, 6H).

Preparation of (7): To the solution of 6 (6 g) in dry THF/MeOD/D₂O=10/2/1 (78 mL) was added NaBD₄ (2.3 g, 54.8 mmol) at r.t. And the reaction mixture was stirred at r.t for 2.5 hr. After completion of reaction, adjusted pH value to 7 with CH₃COOD, after addition of water, the resulting mixture was extracted with EA (100 mL). The combined organic layer was washed with water and brine, dried over Na₂SO₄, and concentrated to give 7 (5.7 g) which was used for the next step. ESI-LCMS: m/z 401 [M+H]⁺.

Preparation of (8): To a solution of 7 (5.7 g) in pyridine (60 mL) was added BzCl (10.0 g, 71.3 mmol) under ice bath. The reaction mixture was stirred at r.t. for 2.5 hrs. LCMS showed 7 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=7/3; Detector, UV 254 nm. This resulted in to give the crude 8 (6.2 g, 8.7 mmol, 57% yield, over two steps) as a white solid. ESI-LCMS: m/z 713 [M+H]⁺.

Preparation of (9): To a solution of 8 (6.2 g, 8.7 mmol) in pyridine (70 mL) and was added 1M NaOH (MeOH/H₂O=4/1) (24 mL). LCMS showed 8 was consumed. The mixture was added saturated NH₄C1 till pH=7.5. The mixture was diluted with water and EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=67/33 Detector, UV 254 nm. This resulted in to give the product 10 (4.3 g, 8.5 mmol, 98% yield) as a white solid. ESI-LCMS: m/z 505 [M+H]⁺; ¹H-NMR (400 MHz, DMSO-d₆): δ 11.23 (m, 1H), 8.77 (m, 2H), 8.06-8.04 (m, 2H), 7.66-7.63 (m, 2H), 7.57-7.53 (m, 3H), 6.16-6.14 (d, J=8.0 Hz, 1H), 5.17 (m, 1H), 4.60-4.52 (m, 2H), 3.34 (m, 1H), 0.93 (m, 9H), 0.14 (m, 6H).

Preparation of (10): To a stirred solution of 9 (4.3 g, 8.5 mmol) in pyridine (45 mL) were added DMTrCl (3.3 g, 9.8 mmol) at r.t. And the reaction mixture was stirred at r.t for 2.5 hr. With ice-bath cooling, the reaction was quenched with water and the product was extracted into EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=97/3 Detector, UV 254 nm. This resulted in to give the product 10 (6.5 g, 8.1 mmol, 95% yield) as a white solid. ESI-LCMS: m/z 807 [M+H]⁺; ¹H-NMR (400 MHz, DMSO-d₆): δ 11.23 (m, 1H), 8.70-8.68 (m, 2H), 8.04-8.02 (m, 2H), 7.66-7.62 (m, 1H), 7.56-7.52 (m, 2H), 7.35-7.26 (m, 2H), 7.25-7.17 (m, 7H), 6.85-6.82 (m, 4H), 6.18-6.16 (d, J=8.0 Hz, 1H), 4.73-4.70 (m, 1H), 4.61-4.58 (m, 1H), 3.71 (m, 6H), 3.32 (m, 1H), 0.83 (m, 9H), 0.09-0.03 (m, 6H).

Preparation of (11): To a solution of 10 (3.5 g, 4.3 mmol) in THF (35 mL) was added 1 M TBAF solution (5 mL). The reaction mixture was stirred at r.t. for 1.5 h. LCMS showed 10 was consumed completely. Water (100 mL) was added. The product was extracted with EA (100 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 20 min, the eluted product was collected at CH₃CN/H₂O (0.5% NH₄HCO₃)=62/38; Detector, UV 254 nm. This resulted in to give 11 (2.7 g, 3.9 mmol, 90.7%) as a white solid. ESI-LCMS: m/z 693 [M+H]⁺.

Preparation of Example 29 monomer: To a suspension of 11 (2.7 g, 3.9 mmol) in DCM (30 mL) was added DCI (0.39 g, 3.3 mmol) and CEP[N(iPr)₂]₂ (1.4 g, 4.7 mmol). The mixture was stirred at r.t. for 2 h. LC-MS showed 11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=73/27; Detector, UV 254 nm. This resulted in to give Example 29 monomer (3.3 g, 3.7 mmol, 94.9%) as a white solid. ESI-LCMS: m/z 893 [M+H]⁺; ¹H-NMR (400 MHz, DMSO-d₆): δ=11.24 (m, 1H), 8.66-8.64 (m, 2H), 8.06-8.03 (m, 2H), 7.65-7.53 (m, 3H), 7.42-7.38 (m, 2H), 7.37-7.34 (m, 2H), 7.25-7.19 (m, 7H), 6.86-6.80 (m, 4H), 6.20-6.19 (d, J=4.0 Hz, 1H), 4.78 (m, 2H), 4.22-4.21 (m, 1H), 3.92-3.83 (m, 1H), 3.72 (m, 6H), 3.62-3.57 (m, 3H), 2.81-2.78 (m, 1H), 2.64-2.61 (m, 1H), 1.17-1.04 (m, 12H); ³¹P-NMR (162 MHz, DMSO-d₆): δ 149.51, 149.30.

### Example 30. Synthesis of Monomer

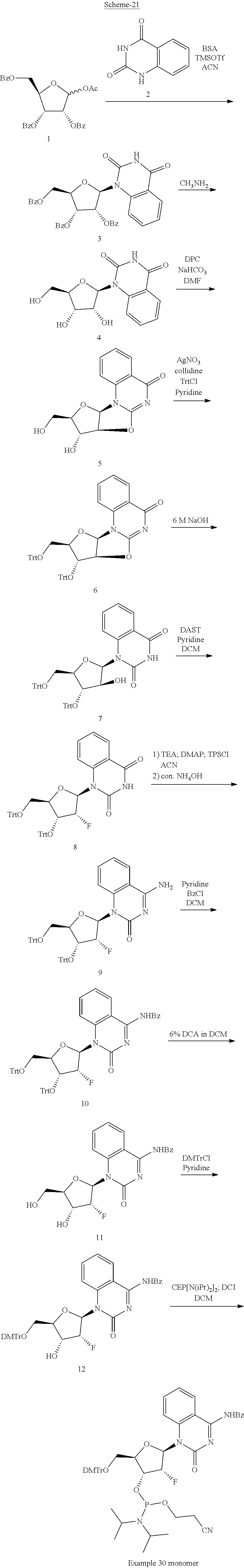

Preparation of (3): To the solution of 1 (70 g, 138.9 mmol) in dry acetonitrile (700 mL) was added 2 (27.0 g, 166.7 mmol), BSA (112.8 g, 555.5 mmol). The mixture was stirred at 50° C. for 1 h. Then the mixture was cooled to −5° C. and TMSOTf (46.2 g, 208.3 mmol) slowly added to the mixture. Then the reaction mixture was stirred at r.t for 48 h. Then the solution was cooled to 0° C. and saturated aq·NaHCO₃ was added and the resulting mixture was extracted with EA. The combined organic layer was washed with water and brine, dried over Na₂SO₄, and concentrated under reduced pressure to give a residue which was purified by silica gel column chromatography (eluent, PE:EA=3:1˜1:1) to give 3 (70 g, 115.3 mmol, 81.6%) as a white solid. ESI-LCMS: m/z 605 [M−H]⁺.

Preparation of (4): To the solution of 3 (70.0 g, 115.3 mmol) in methylammonium solution (1 M, 700 mL), and the reaction mixture was stirred at 40° C. for 15 h. After completion of reaction, the resulting mixture was concentrated. The residue was crystallized from EA. Solid was isolated by filtration, washed with PE and dried overnight at 45° C. in vacuum to give 4 (31.0 g, 105.4 mmol, 91.1%) as a white solid. ESI-LCMS: m/z 295 [M+H]⁺; ¹H-NMR (400 MHz, DMSO): δ 11.63 (s, 1H), 8.07-7.99 (m, 1H), 7.81 (d, J=8.4 Hz, 1H), 7.72-7.63 (m, 1H), 7.34-7.26 (m, 1H), 6.18 (d, J=6.4 Hz, 1H), 5.24 (s, 1H), 5.00 (s, 2H), 4.58-4.47 (m, 1H), 4.19-4.10 (m, 1H), 3.85-3.77 (m, 1H), 3.75-3.66 (m, 1H), 3.66-3.57 (m, 1H).

Preparation of (5): To the solution of 4 (20.0 g, 68.0 mmol) in dry DMF (200 mL) was added DPC (18.9 g, 88.0 mmol) and NaHCO₃ (343 mg, 4 mmol) at r.t, and the reaction mixture was stirred at 150° C. for 35 min. After completion of reaction, the resulting mixture was poured into tert-Butyl methyl ether (4 L). Solid was isolated by filtration, washed with PE and dried □ in vacuum to give crude 5 (21.0 g) as a brown solid which was used directly for next step (ref for 5, Journal of Organic Chemistry, 1989, vol. 33, p. 1219-1225). ESI-LCMS: m/z 275 [M−H].

Preparation of (6): To the solution of 5 (crude, 21.0 g) in Pyridine (200 mL) was added AgNO₃ (31.0 g, 180.0 mmol) and collidine (88.0 g, 720 mmol) and TrtCl (41.5 g, 181 mmol) at r.t, and the reaction mixture was stirred at r.t for 15 h. After addition of water, the resulting mixture was extracted with EA. The combined organic layer was washed with water and brine, dried over Na₂SO₄, and concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (10.0 g, 13.1 mmol, 20% yield over 3 steps) as a white solid. ESI-LCMS: m/z 761 [M+H]⁺.

Preparation of (7): To the solution of 6 (10.0 g, 13.1 mmol) in THF (100 mL) was added 6N NaOH (30 mL) at r.t, and the reaction mixture was stirred at r.t for 1 hr. After addition of NH₄Cl, the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in to give 7 (9.3 g, 11.9 mmol, 90%) as a white solid. ESI-LCMS: m/z 777 [M−H]⁻; ¹H-NMR (400 MHz, DMSO-d₆): δ 11.57 (s, 1H), 8.02 (d, J=8.7 Hz, 1H), 7.88-7.81 (m, 1H), 7.39-7.18 (m, 30H), 7.09-6.99 (m, 30H), 6.92-6.84 (m, 30H), 6.44 (d, J=4.0 Hz, 1H), 4.87 (d, J=4.0 Hz, 1H), 4.37-4.29 (m, 1H), 4.00-3.96 (m, 1H), 3.76-3.70 (m, 1H), 3.22-3.13 (m, 1H), 3.13-3.04 (m, 1H).

Preparation of (8): To the solution of 7 (8.3 g, 10.7 mmol) in dry DCM (80 mL) was added Pyridine (5.0 g, 64.2 mmol) and DAST (6.9 g, 42.8 mmol) at 0° C., and the reaction mixture was stirred at r.t for 15 hr. After addition of NH₄Cl, the resulting mixture was extracted with DCM. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (6.8 g, 8.7 mmol, 81.2%) as a white solid. ESI-LCMS: m/z 779 [M−H]⁺; ¹⁹F-NMR (376 MHz, DMSO-d₆): δ −183.05.

Preparation of (9): To the solution of 8 (5.8 g, 7.5 mmol) in dry ACN (60 mL) was added TEA (1.5 g, 15.1 mmol), DMAP (1.84 g, 15.1 mmol) and TPSCl (4.1 g, 13.6 mmol) at r.t, and the reaction mixture was stirred at room temperature for 3 h under N₂ atmosphere. After completion of reaction, the mixture was added NH₃·H₂O (12 mL). After addition of water, the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 9 (5.5 g, 7 mmol, 90.2%) as a white solid. ESI-LCMS: m/z 780 [M+H]⁺.

Preparation of (10): To a solution of 9 (5.5 g, 7 mmol) in DCM (50 mL) with an inert atmosphere of nitrogen was added pyridine (5.6 g, 70.0 mmol) and BzCl (1.2 g, 8.5 mmol) in order at 0° C. The reaction solution was stirred for 30 minutes at room temperature. The solution was diluted with DCM (100 mL) and the combined organic layer was washed with water and brine, dried over Na₂SO₄, and concentrated under reduced pressure to give a residue which was purified by silica gel column chromatography (eluent, PE:EA=5:1˜2:1) to give 10 (5.4 g, 6.1 mmol, 90.6%) as a white solid. ESI-LCMS: m/z 884 [M+H]⁺; ¹⁹F-NMR (376 MHz, DMSO-d₆): δ −183.64.

Preparation of (11): To the solution of 10 (5.4 g, 6.1 mmol) in the solution of DCA (6%) in DCM (60 mL) was added TES (15 mL) at r.t, and the reaction mixture was stirred at room temperature for 5-10 min. After completion of reaction, the resulting mixture was added NaHCO₃, the resulting mixture was extracted with DCM. The combined organic layer was washed with water and brine, dried over Na₂SO₄, and concentrated under reduced pressure and the residue was crystallized from EA. Solid was isolated by filtration, washed with PE and dried overnight at 45° Qin vacuum to give 11 (2.0 g, 5.0 mmol, 83.2%) as a white solid. ESI-LCMS: m/z 400 [M+H]⁺.

Preparation of (12): To a solution of 11 (2.0 g, 5.0 mmol) in dry Pyridine (20 mL) was added DMTrCl (2.0 g, 6.0 mmol). The reaction mixture was stirred at r.t. for 2.5 h. LCMS showed 11 was consumed and water (200 mL) was added. The product was extracted with EA (200 mL) and the organic layer was washed with brine and dried over Na₂SO₄ and concentrated to give the crude. The crude was purified by c.c. (PE:EA=4:1˜1:1) to give crude 12. The crude was further purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 12 (2.1 g, 3 mmol, 60%) as a white solid. ESI-LCMS: m/z 702 [M+H]⁺; ¹H-NMR (400 MHz, DMSO-d₆): δ 12.63 (s, 1H), 8.54 (d, J=7.8 Hz, 1H), 8.25 (d, J=7.2 Hz, 2H), 7.82 (d, J=3.6 Hz, 2H), 7.67-7.58 (m, 1H), 7.57-7.49 (m, 2H), 7.49-7.39 (m, 1H), 7.39-7.31 (m, 2H), 7.27-7.09 (m, 7H), 6.82-6.69 (m, 4H), 6.23 (d, J=26.1 Hz, 1H), 5.59-5.49 (m, 1H), 4.83-4.61 (m, 1H), 4.15-4.01 (m, 1H), 3.74-3.59 (m, 6H), 3.33-3.28 (m, 1H), 3.16-3.05 (m, 1H). ¹⁹F-NMR (376 MHz, DMSO-d₆): δ −191.66.

Preparation of Example 30 monomer: To a suspension of 12 (2.1 g, 3.0 mmol) in DCM (20 mL) was added DCI (310 mg, 2.6 mmol) and CEP[N(iPr)₂]₂ (1.1 g, 3.7 mmol). The mixture was stirred at r.t. for 1 h. LC-MS showed 12 was consumed completely. The solution was washed with water twice and washed with brine and dried over Na₂SO₄. Then concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 30 monomer (2.1 g, 2.3 mmol, 80.0%) as a white solid. ESI-LCMS: m/z 902 [M+H]⁺; ¹H-NMR (400 MHz, DMSO-d₆): δ 12.64 (s, 1H), 8.54 (d, J=7.6 Hz, 1H), 8.24 (d, J=7.7 Hz, 2H), 7.93-7.88 (m, 2H), 7.67-7.58 (m, 1H), 7.56-7.42 (m, 3H), 7.41-7.29 (m, 2H), 7.27-7.08 (m, 7H), 6.82-6.64 (m, 4H), 6.37-6.18 (m, 1H), 6.03-5.72 (m, 1H), 5.26-4.83 (m, 1H), 4.28-4.12 (m, 1H), 3.88-3.72 (m, 1H), 3.71-3.37 (m, 9H), 3.15-3.00 (m, 1H), 2.83-2.75 (m, 1H), 2.66-2.57 (m, 1H), 1.21-0.88 (m, 12H). ¹⁹F-NMR (376 MHz, DMSO-d₆): δ −189.71. ³¹P-NMR (162 MHz, DMSO-d₆): δ 149.48, 149.50, 148.95, 148.88.

### Example 31. Synthesis of Monomer

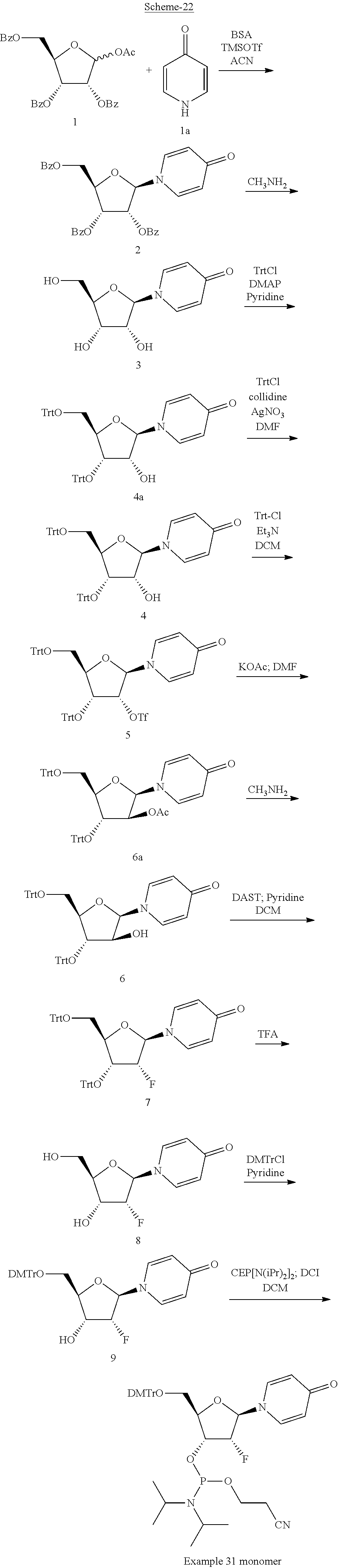

Preparation of (2): To a solution of 1 (40.0 g, 79.3 mmol), 1a (7.6 g, 80.1 mmol) in ACN (100 mL). Then added BSA (35.2 g, 174.4 mmol) under N₂ atmosphere. The mixture was stirred at 50° C. for 1 h until the solution was clear. Then cool down to 0° C. and dropped TMSOTf (18.5 g, 83.2 mmol). The mixture was stirred at 75° C. for 1 h, TLC showed 1 was consumed completely. Then the solution was diluted with EA, washed with H₂O twice. The solvent was concentrated under reduced pressure and the residue was used for next step. ESI-LCMS: m/z 540 [M+H]⁺.

Preparation of (3): To a solution of 2 (37.1 g, 68.7 mmol) in 30% CH₂NH₂/MeOH solution (200 mL). The mixture was stirred at 25° C. for 2 h. TLC showed 2 was consumed completely. The solvent was concentrated under reduced pressure and the residue was washed with EA twice to give 3 (12.5 g, 55.2 mmol) (ref for intermediate 3 Bioorganic & Medicinal Chemistry Letters, 1996, Vol. 6, No. 4, pp. 373-378,) which was used directly for the next step. ESI-LCMS: m/z 228 [M+H]⁺.

Preparation of (4): To a solution of 3 (12.5 g, 55.2 mmol) in pyridine (125 mL) and added DMAP (1.3 g, 11.0 mmol), TrtCl (30.7 g, 110.5 mmol). The mixture was stirred at r.t. for 24 h. TLC showed 3 was consumed completely. H₂O was added to the mixture. Then filtered and the solution diluted with EA. The organic layer was washed with NaHCO₃ and brine. The solvent was concentrated under reduced pressure and then added ACN, filtered to give 4a (17.0 g, 35.4 mmol, 64% yield) as a white solid.

To a solution of 4a (17.0 g, 35.4 mmol) in DMF (200 mL), collidine (5.2 g, 43.5 mmol), TrCl (13.1 g, 47.1 mmol) were added after 2 h and then again after 3 h TrCl (13.1 g, 47.1 mmol), AgNO₃ (8.0 g, 47.1 mmol). The mixture was stirred at 25° C. for 24 h. TLC showed 4a was consumed completely. Then filtered and the solution diluted with EA. The organic layer was washed with NaHCO₃ and brine. The solvent was concentrated under reduced pressure and then added ACN, filtered to get 4 (14.2 g, 19.5 mmol, 54% yield) as a white solid. ESI-LCMS: m/z 712 [M+H]⁺; ¹H-NMR (400 MHz, DMSO-d₆): δ 7.83 (d, J=8 Hz, 2H), 7.42-7.20 (m, 30H), 6.18 (d, J=7 Hz, 1H), 6.09 (d, J=8 Hz, 2H), 5.60 (d, J=7 Hz, 1H), 4.22 (m, 1H), 3.90 (d, J=5 Hz, 1H), 2.85 (d, J=10 Hz, 1H), 2.76 (s, 1H), 2.55-2.50 (dd, 1H).

Preparation of (5): To a solution of 4 (14.2 g, 19.9 mmol) in DCM (150 mL), DMAP (2.4 g, 19.9 mmol), TEA (4.0 g, 39.9 mmol, 5.6 mL) were added. Then cool down to 0° C., TfCl (6.7 g, 39.9 mmol) dissolved in DCM (150 mL) were dropped. The mixture was stirred at 25° C. for 1 h. TLC showed 4 was consumed completely. Then filtered and the solution diluted with EA. The organic layer was washed with NaHCO₃ and brine. The solvent was concentrated under reduced pressure to get 5 (16.8 g, 19.9 mmol) as a brown solid. ESI-LCMS: m/z 844 [M+H]⁺.

Preparation of (6): To a solution of 5 (16.8 g, 19.9 mmol) in DMF (200 mL), KOAc (9.7 g, 99.6 mmol) were added, The mixture was stirred at 25° C. for 14 h and 50° C. for 3 h, TLC showed 5 was consumed completely. Then filtered and the solution diluted with EA. The organic layer was washed with H₂O and brine. The solvent was concentrated under reduced pressure to get 6a (15.0 g, 18.9 mmol, 90% yield) as a brown solid. To a solution of 6a (15.0 g, 19.9 mmol) in 30% CH₃NH₂/MeOH solution (100 mL) were added. The mixture was stirred at 25° C. for 2 h, TLC showed 6a was consumed completely. Then the solvent was concentrated under reduced pressure and the residue was purified by cc (0-5% MeOH in DCM) to give 6 (11.6 g, 16.3 mmol, 82% yield) as a yellow solid. ESI-LCMS: m/z 712 [M+H]⁺; ¹H-NMR (400 MHz, DMSO-d₆): δ 7.59 (d, J=8 Hz, 2H), 7.37-7.22 (m, 30H), 6.01 (d, J=8 Hz, 2H), 5.84 (d, J=3 Hz, 1H), 5.42 (d, J=4 Hz, 1H), 3.78-3.70 (m, 3H), 3.10 (t, J=9 Hz, 1H), 2.53 (d, J=4 Hz, 6H), 1.77 (s, 6H).

Preparation of (7): To a solution of 6 (11.6 g, 16.32 mmol) in DCM (200 mL), DAST (7.9 g, 48.9 mmol) were added at 0° C., The mixture was stirred at 25° C. for 16 h, TLC showed 6 was consumed completely. Then the solution was diluted with EA, washed with NaHCO₃ twice, The solvent was concentrated under reduced pressure the residue purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in to give 7 (11.6 g, 13.8 mmol, 84% yield) as a white solid. ESI-LCMS: m/z 714 [M+H]⁺.

Preparation of (8): To a solution of 7 (11.6 g, 16.2 mmol) in DCM (100 mL) was added TFA (10 mL). The mixture was stirred at 20° C. for 1 h. TLC showed 7 was consumed completely. Then the solution was concentrated under reduced pressure the residue was purified by silica gel column (0˜20% MeOH in DCM) and Flash-Prep-HPLC with the following conditions (IntelFlash-1): Column, C18 silica gel; mobile phase, CH₃CN/H₂O (0.5% NH₄HCO₃)=0/1 increasing to CH₃CN/H₂O (0.5% NH₄HCO₃)=1/3 within 25 min, the eluted product was collected at CH₃CN/H₂O (0.5% NH₄HCO₃)=0/1; Detector, UV 254 nm. This resulted in to give 9 (1.7 g, 7.2 mmol, 45% yield) as a white solid. ESI-LCMS: m/z 229.9 [M+H]⁺; ¹H-NMR (400 MHz, DMSO-d₆): δ 7.91 (d, J=8 Hz, 2H), 6.14 (d, J=8 Hz, 2H), 5.81-5.76 (m, 2H), 5.28 (t, J=5 Hz, 1H), 5.13-4.97 (t, J=4 Hz, 1H), 4.23 (m, 1H), 3.97 (m, 1H), 3.74-3.58 (m, 2H); ¹⁹F-NMR (376 MHz, DMSO-d₆): δ −206.09.

Preparation of (9): To a solution of 8 (1.4 g, 6.1 mmol) in pyridine (14 mL) was added DMTrCl (2.5 g, 7.3 mmol) at 20° C. The mixture was stirred at 20° C. for 1 h. TLC showed 8 was consumed completely. Water was added to the reaction. The product was extracted with EA, The organic layer was washed with NaHCO₃ and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=1/1; Detector, UV 254 nm. This resulted in to give 9 (2.5 g, 4.6 mmol, 76 yield) as a white solid. ESI-LCMS: m/z 532.2 [M+H]⁺; ¹H-NMR (400 MHz, DMSO-d₆): δ 7.87-7.84 (m, 2H), 7.40-7.22 (m, 9H), 6.91-6.87 (m, 4H), 5.98-5.95 (m, 2H), 5.88-5.77 (m, 2H), 5.16-5.02 (m, 1H), 4.42 (m, 1H), 4.05 (m, 1H), 3.74 (s, 6H), 3.35 (m, 2H); ¹⁹F-NMR (376 MHz, DMSO-d₆): δ −202.32.

Preparation of Example 31 monomer: To a solution of 9 (2.2 g, 4.1 mmol) in DCM (20 mL) was added DCI (415 mg, 3.5 mmol) and CEP (1.5 g, 4.9 mmol) under N₂ pro. The mixture was stirred at 20° C. for 0.5 h. TLC showed 9 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give Example 31 monomer (2.6 g, 3.5 mmol, 85% yield) as a white solid. ESI-LCMS: m/z 732.2 [M+H]⁺; ¹H-NMR (400 MHz, DMSO-d₆): δ 7.87-7.84 (m, 2H), 7.40-7.22 (m, 9H), 6.91-6.87 (m, 4H), 5.98-5.95 (m, 2H), 5.90-5.88 (m, 1H), 5.30-5.17 (m, 1H), 4.62 (m, 1H), 4.19 (m, 1H), 3.78-3.73 (m, 7H), 3.62-3.35 (m, 5H), 2.78 (t, J=5 Hz, 1H), 2.63 (t, J=6 Hz, 1H), 1.14-0.96 (m, 12H); ¹⁹F-NMR (376 MHz, DMSO-d₆): δ −200.77, 200.80, 201.62, 201.64. ³¹P-NMR (162 MHz, DMSO-d₆): δ 150.31, 150.24, 149.66, 149.60.

### Example 32. Synthesis of End Cap Monomer

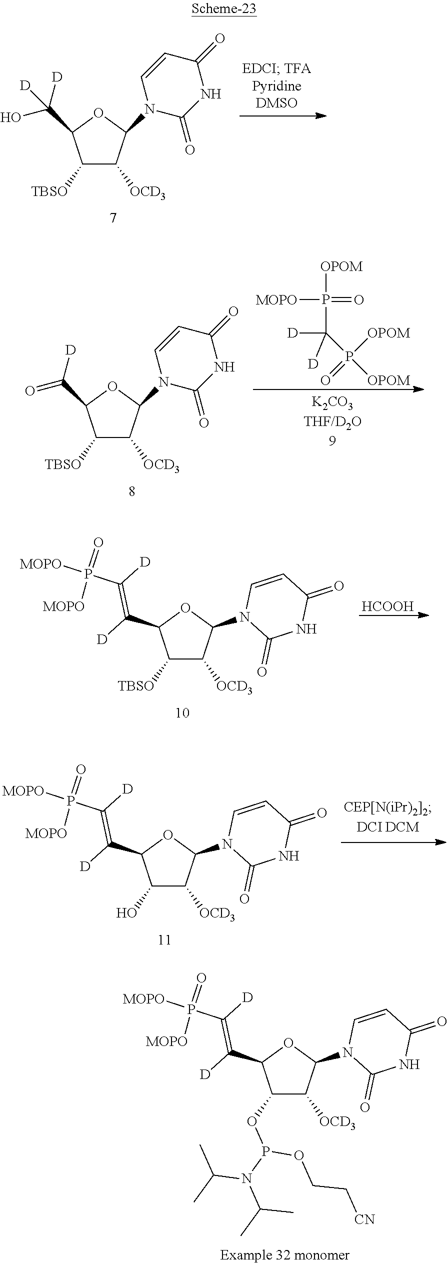

Preparation of (8): To a stirred solution of 7 (13.4 g, 35.5 mmol, Scheme 5) in DMSO (135 mL) were added EDCI (6.3 g, 32.9 mmol) and pyridine (0.9 g, 10.9 mmol), TFA (0.6 g, 5.5 mmol) at r.t. And the reaction mixture was stirred at r.t for 2 h. LCMS showed 7 consumed completely. The reaction was quenched with water and the product was extracted with EA (1800 mL). The organic phase was washed by brine, dried over Na₂SO₄, The organic phase was evaporated to dryness under reduced pressure to give a residue 8 (13.2 g, 35.3 mmol, 99.3% yield). Which was used directly to next step. ESI-LCMS: m/z=375 [M+H₂O]⁺

Preparation of (10): A solution of 8 (13.2 g, 35.3 mmol), 9 (26.8 g, 42.3 mmol, Scheme 18) and K2CO3 (19.5 g, 141.0 mmol) in dry THF (160 mL) and D₂O (53 mL) was stirred at r.t. 17 h. LCMS showed most of 8 was consumed. The product was extracted with EA (2500 mL) and the organic layer was washed with brine and dried over Na₂SO₄. Then the organic layer was concentrated to give a residue which was purified by c.c. (PE:EA=10:1˜1:2) to give product 10 (8.1 g, 11.8 mmol, 33.4%0 yield) as a white solid. ESI-LCMS m/z=682 [M+H]⁺; ¹H-NMR (400 MHz, DMSO-d₆): δ 11.42 (s, 1H), 7.69-7.71 (d, J=8.1 Hz, 1H), 5.78-5.79 (d, J=3.7 Hz, 1H), 5.65-5.67 (m, 1H), 5.59-5.63 (m, 4H), 4.29-4.35 (m, 2H), 3.97-3.99 (m, 1H), 1.15 (s, 18H), 0.87 (s, 9H), 0.07-0.08 (d, J=5.1 Hz, 6H). ³¹P-NMR (162 MHz, DMS O-d₆) δ 16.62.

Preparation of (11): To a round-bottom flask was added 10 (7.7 g, 11.1 mmol) in a mixture of HCOOH (80 mL) and H₂O (80 mL). The reaction mixture was stirred at 40° C. for 3 h. LCMS showed the 10 was consumed completely. The reaction mixture was adjusted the pH=7.0 with con·NH₃·H₂O (100 mL). Then the mixture was extracted with DCM (100 mL*3). The combined DCM layer was dried over Na₂SO₄. Filtered and filtrate was concentrated to give crude which was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/1 within 20 min, the eluted product was collected at CH₃CN/H₂O (0.5% NH₄HCO₃)=1/0; Detector, UV 254 nm. To give product 11 (5.5 g, 9.6 mmol, 86.1% yield) as a white solid. ESI-LCMS m/z=568 [M+H]⁺; ¹H-NMR (400 MHz, DMSO-d₆): δ 11.42 (s, 1H, exchanged with D₂O), 7.62-7.64 (d, J=8.1, 1H), 5.81-5.82 (d, J=4.3, 1H), 5.58-5.66 (m, 5H), 5.52-5.53 (d, J=6.6, 1H), 4.34-4.37 (m, 1H), 4.09-4.13 (m, 1H), 3.94-3.96 (t, J=9.7, 1H), 1.15 (s, 18H), 0 (s, 1H). ³¹P NMR (162 MHz, DMSO-d₆) δ 17.16.

Preparation of Example 32 monomer: To a solution of 11 (5.3 g, 9.3 mmol) in DCM (40 mL) was added the DCI (1.1 g, 7.9 mmol), then CEP[N(ipr)₂]₂ (3.4 g, 11.2 mmol) was added. The mixture was stirred at r.t. for 1 h. LCMS showed 11 consumed completely. The reaction mixture was washed with H₂O (50 mL*2) and brine (50 mL*1). Dried over Na₂SO₄ and concentrated to give crude which was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. The product was concentrated to give Example 32 monomer (6.2 g, 8.0 mmol, 85.6% yield) as a white solid. ESI-LCMS m/z=768 [M+H]⁺; ¹H-NMR (400 MHz, DMSO-d₆): δ 11.43 (s, 1H), 7.68-7.71 (m, 1H), 5.79-5.81 (m, 1H), 5.58-5.67 (m, 5H), 4.34-4.56 (m, 2H), 4.14-4.17 (m, 1H), 3.54-3.85 (m, 4H), 2.78-2.81 (m, 2H), 1.13-1.17 (m, 30H). ³¹P-NMR (162 MHz, DMSO-d₆): δ 149.66, 149.16, 16.84, 16.56.

### Example 33. Synthesis of Monomer

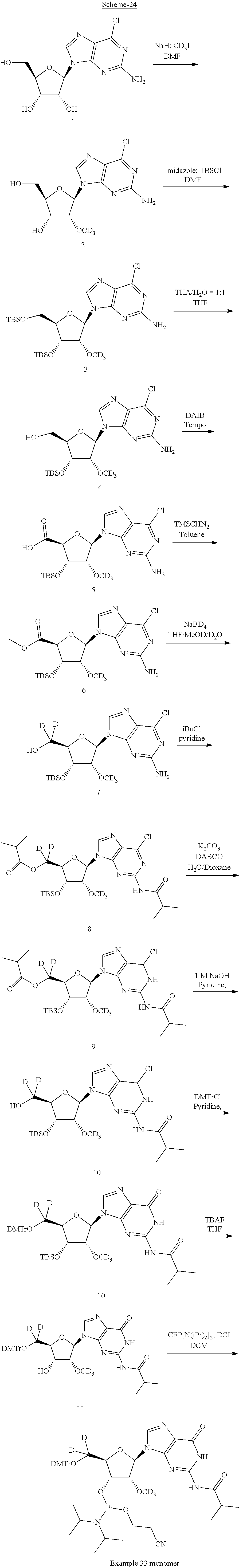

Preparation of (2): To a solution of 1 (20.0 g, 66.4 mmol) in dry DMF (400 mL) was added sodium hydride (1.9 g, 79.7 mmol) for 30 min, then was added CD₃I (9.1 g, 79.7 mmol) in dry DCM (40 mL) at −20° C. for 5.5 hr. LCMS showed the reaction was consumed. The mixture was filtered and the clear solution was evaporated to dryness and was evaporated with CH₃OH. The crude was purified by silica gel column (SiO2, DCM/MeOH=50:1˜10:1). This resulted in to give the product 2 (7.5 g, 23.5 mmol, 35.5% yield) as a solid. ESI-LCMS: m/z 319 [M+H]⁺; ¹H-NMR (400 MHz, DMSO-d₃): δ=8.38 (m, 1H), 6.97 (m, 2H), 5.93-5.81 (m, 1H), 5.27-5.26 (d, J=4 Hz, 1H), 5.13-5.11 (m, 1H), 4.39-4.31 (m, 1H), 4.31-4.25 (m, 1H), 3.96-3.94 (m, 1H), 3.66-3.63 (m, 1H), 3.63-3.56 (m, 1H).

Preparation of (3): To a solution of 2 (7.5 g, 23.5 mmol) in dry DMF (75 mL) was added Imidazole (5.6 g, 82.3 mmol) and TBSCl (8.9 g, 58.8 mmol). The mixture was stirred at r.t. over night. LCMS showed 2 was consumed completely. The reaction was quenched with water (300 mL). The product was extracted into ethyl acetate (100 mL). The organic layer was washed with brine and dried over anhydrous Na₂SO₄. The solvent was removed to give the cured 3 (9.8 g) as a solid which used for the next step. ESI-LCMS: m/z 547 [M+H]⁺.

Preparation of (4): To a solution of 3 (9.8 g) in THF (40 mL) was added TFA (10 mL) and water (10 mL) at 0° C. The reaction mixture was stirred at 0° C. for 5 h. LC-MS showed 3 was consumed completely. Con. NH₄OH was added to the mixture at 0° C. to quench the reaction until the pH=7.5. The product was extracted into ethyl acetate (200 mL). The organic layer was washed with brine and dried over anhydrous Na₂SO₄. The solvent was removed to give the cured 4 (8.4 g) as a solid which used for the next step. ESI-LCMS: m/z 433 [M+H]⁺.

Preparation of (5): To a solution of 4 (8.4 g) in DCM/H₂O=2:1 (84 mL) was added DAIB (18.8 g, 58.4 mmol) and TEMPO (0.87 g, 5.8 mmol). The reaction mixture was stirred at 40° C. for 2 h. LCMS showed 4 was consumed. The mixture was diluted with DCM and water was added. The product was extracted with DCM. The organic layer was washed with brine and dried over anhydrous Na₂SO₄. The solution was then concentrated under reduced pressure. This resulted in to give 5 (14.4 g) as a white solid. ESI-LCMS: m/z 447 [M+H]⁺.

Preparation of (6): To a solution of 5 (14.4 g) in toluene (90 mL) and methanol (60 mL) was added 2M TMSCHN₂ (8.9 g, 78.1 mmol) till the yellow color not disappear at r.t. for 10 min. LCMS showed 5 was consumed. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=65/35 Detector, UV 254 nm. This resulted in to give the product 6 (3.5 g, 7.6 mmol, 32.3% yield over three steps, 70% purity) as a white solid. ESI-LCMS: m/z 461 [M+H]⁺.

Preparation of (7): To the solution of 6 (3.5 g, 7.6 mmol) in dry THF/MeOD/D₂O=10/2/1 (45 mL) was added NaBD₄ (0.96 g, 22.8 mmol). And the reaction mixture was stirred at r.t for 2.5 hr. After completion of reaction, the resulting mixture was added CH₃COOD to pH=7, after addition of water, the resulting mixture was extracted with EA (100 mL). The combined organic layer was washed with water and brine, dried over Na₂SO₄, and concentrated to give 7 (3.3 g) which was used for the next step. ESI-LCMS: m/z 435 [M+H]⁺.

Preparation of (8): To a solution of 7 (3.3 g) in dry DCM (30 mL) was added pyridine (5.9 g, 74.5 mmol) and iBuCl (2.4 g, 22.4 mmol) in DCM (6 mL) under ice bath. The reaction mixture was stirred at 0° C. for 2.5 hr. LCMS showed 7 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=87/13; Detector, UV 254 nm. This resulted in to give the crude 8 (1.6 g, 2.8 mmol, 36.8% yield over two steps) as a white solid. ESI-LCMS: m/z 575 [M+H]⁺.

Preparation of (9): To a solution of 8 (1.6 g, 2.8 mmol,) in H₂O/dioxane=1:1 (30 ml) was added K2CO3 (772.8 mg, 5.6 mmol) and DABCO (739.2 mg, 2.9 mmol). The reaction mixture was stirred at 50° C. for 3 hr. LCMS showed 8 was consumed. The mixture was diluted with EA and water was added. The product was extracted with EA. The combined organic layer was washed with water and brine, dried over Na₂SO₄, and concentrated to give 9 (1.8 g) which was used for the next step. ESI-LCMS: m/z 557 [M+H]⁺.

Preparation of (10): To a solution of 9 (1.8 g) in pyridine (20 mL) and was added 2M NaOH (MeOH/H₂O=4/1) (5 mL) at 0° C. for 1 h. LCMS showed 9 was consumed. The mixture was added saturated NH₄C1 till pH=7.5. The mixture was diluted with water and EA. The organic layer was washed with brine and dried over Na₂SO₄ and concentrated to give the crude. This resulted in to give the product 10 (1.5 g) as a white solid which was used for the next step. ESI-LCMS: m/z 487 [M+H]⁺.

Preparation of (11): To a stirred solution of 10 (1.5 g) in pyridine (20 mL) were added DMTrCl (1.1 g, 3 mmol) at r.t. And the reaction mixture was stirred at r.t for 2.5 hr. With ice-bath cooling, the reaction was quenched with water and the product was extracted into EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=7/3 Detector, UV 254 nm. This resulted in to give the product 11 (1.9 g, 2.4 mmol, 85.7% yield over two steps) as a white solid. ESI-LCMS: m/z 789.3 [M+H]⁺; ¹H-NMR (400 MHz, DMSO-d₆): δ 12.10 (m, 1H), 11.63 (m, 1H), 8.20 (m, 1H), 7.35-7.33 (m, 2H), 7.29-7.19 (m, 7H), 6.86-6.83 (m, 4H), 5.89-5.88 (d, J=4 Hz, 1H), 4.40-4.28 (m, 2H), 3.72 (m, 6H), 2.81-2.76 (m, 1H), 1.13-1.11 (m, 6H), 0.80 (m, 9H), 0.05-0.01 (m, 7H).

Preparation of (12): To a solution of 11 (1.9 g, 2.4 mmol) in THF (20 mL) was added 1 M TBAF solution (3 mL). The reaction mixture was stirred at r.t. for 1.5 h. LCMS showed 11 was consumed completely. Water (100 mL) was added. The product was extracted with EA (50 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 20 min, the eluted product was collected at CH₃CN/H₂O (0.5% NH₄HCO₃)=58/42; Detector, UV 254 nm. This resulted in to give 12 (1.5 g, 2.2 mmol, 91.6% yield) as a white solid. ESI-LCMS: m/z 675.3 [M+H]⁺; ¹H-NMR (400 MHz, DMSO-d₆): δ 12.09 (m, 1H), 11.60 (m, 1H), 8.14 (m, 1H), 7.35-7.27 (m, 2H), 7.25-7.20 (m, 7H), 6.85-6.80 (m, 4H), 5.96-5.94 (d, J=8 Hz, 1H), 5.26-5.24 (m, 1H), 4.35-4.28 (m, 2H), 3.72 (m, 6H), 3.32 (m, 1H), 2.79-2.72 (m, 1H), 1.13-1.11 (m, 6H).

Preparation of Example 33 monomer: To a suspension of 11 (1.5 g, 2.2 mmol) in DCM (15 mL) was added DCI (220.8 mg, 1.9 mmol) and CEP[N(iPr)₂]₂ (795.7 mg, 2.6 mmol) under N₂ pro. The mixture was stirred at r.t. for 2 h. LCMS showed 11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=4/1; Detector, UV 254 nm. This resulted in to give Example 33 monomer (1.6 g, 1.8 mmol, 83% yield) as a white solid. ESI-LCMS: m/z 875 [M+H]⁺; ¹H-NMR (400 MHz, DMSO-d₆): δ 12.12 (m, 1H), 11.60 (m, 1H), 8.15 (m, 1H), 7.37-7.29 (m, 2H), 7.27-7.20 (m, 7H), 6.86-6.81 (m, 4H), 5.94-5.88 (m, 1H), 4.54-4.51 (m, 2H), 4.21-4.20 (m, 1H), 3.73-3.54 (m, 1OH), 2.80-2.75 (m, 1H), 2.61-2.58 (m, 1H), 1.19-1.11 (m, 19H). ³¹P-NMR (162 MHz, DMSO-d₆): δ=149.77, 149.71.

### Example 34. Synthesis of Monomer

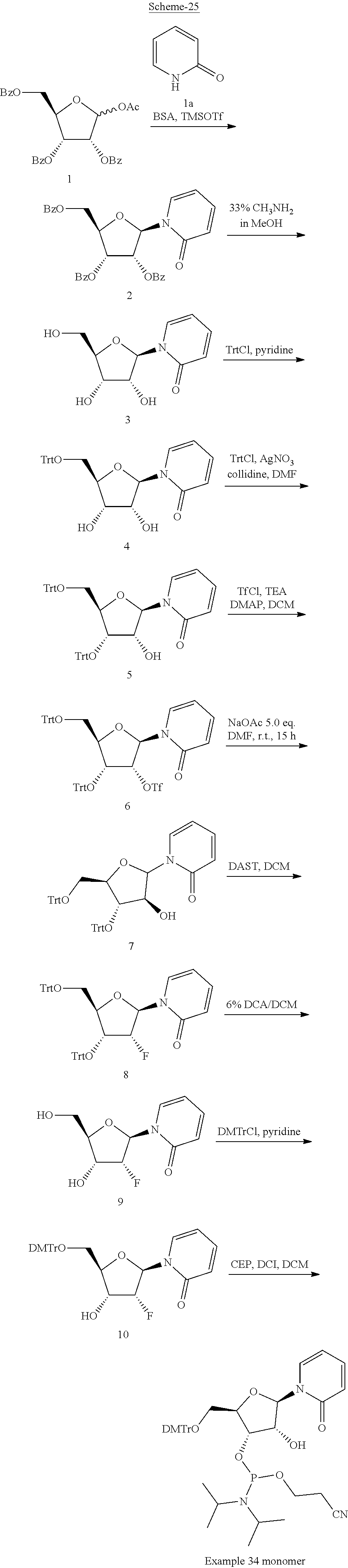

Preparation of (2): To a solution of 1 (50.0 g, 99.2 mmol) and 1a (11.3 g, 119.0 mmol) in ACN (500.0 mL). Then added BSA (53.2 g, 218.0 mmol) under N₂ Pro. The mixture was stirred at 50° C. for 1 h until the solution was clear. Then cool down to 0° C. and dropped TMSOTf (26.4 g, 119.0 mmol). The mixture was stirred at 75° C. for 1 h, TLC showed 1 was consumed completely. The reaction was quenched by sodium bicarbonate solution at 0° C., then the solution was diluted with EA, washed with H₂O twice. The solvent was concentrated under reduced pressure and the crude 2 (60.1 g) was used for next step. ESI-LCMS: m/z 540.2 [M+H]⁺.

Preparation of (3): To a solution of 2 (60.1 g) in CH₃NH₂/ethanol (500.0 mL). The mixture was stirred at 25° C. for 2 h. TLC showed 2 was consumed completely. The solvent was concentrated under reduced pressure and the residue was purified by c.c. (MeOH:DCM=50:1˜10:1) to give 3 (22.0 g, 96.9 mmol, 97.3% yield over two steps). ESI-LCMS: m/z 228.0 [M+H]⁺; ¹H-NMR (400 MHz, DMSO-d₆): δ 8.01-7.98 (m, 1H), 7.43-7.38 (m, 1H), 6.37-6.35 (m, 1H), 6.27-6.23 (m, 1H), 6.03 (d, J=3.5 Hz, 1H), 5.39 (d, J=4.2 Hz, 1H), 5.11 (t, J=5.1 Hz, 1H), 5.03 (d, J=5.1 Hz, 1H), 3.98-3.95 (m, 2H), 3.91-3.88 (m, 1H), 3.74-3.57 (m, 2H).

Preparation of (4): To a solution of 3 (22.0 g, 96.9 mmol) in pyridine (250.0 mL), TrtCl (30.7 g, 110.5 mmol) was added. The mixture was stirred at 25° C. for 24 h. TLC showed 3 was consumed completely, H₂O was added to the mixture. Then filtered and the filtrate diluted with EA, the organic layer was washed with NaHCO₃ and brine. The solvent was concentrated under reduced pressure and then purified by c.c. (PE/EA=5:1˜0:1) to give 4 (38.8 g, 82.5 mmol, 85.1% yield) as a white solid. ESI-LCMS: m/z 470.1 [M+H]⁺.

Preparation of (5): To a solution of 4 (38.8 g, 82.5 mmol) in DMF (500.0 mL), collidine (10.0 g, 107.3 mmol), TrtCl (27.6 g, 99.1 mmol) were added followed by AgNO₃ (18.0 g, 105.1 mmol). The mixture was stirred at 25° C. for 4 h. TLC showed 4 was consumed completely. Then filtered and the filtrate diluted with EA. The organic layer was washed with NaHCO₃ and brine. The solvent was concentrated under reduced pressure and then purified by c.c. (PE/EA=5:1˜1:1) to give a mixture of 5 (52.3 g, 73.5 mmol, 86.3% yield) as white solid. ESI-LCMS: m/z 711.1 [M+H]⁺.

Preparation of (6): To a solution of 5 (52.3 g, 73.5 mmol) in DCM (500.0 mL), DMAP (8.9 g, 73.5 mmol), TEA (14.9 g, 147.3 mmol, 20.6 mL) were added, cool down to 0° C., TfCl (16.1 g, 95.6 mmol) dissolved in DCM (100.0 mL) were dropped. The mixture was stirred at 25° C. for 1 h. TLC showed 5 was consumed completely. Then filtered and the solution diluted with EA. The organic layer was washed with NaHCO₃ and brine. The solvent was concentrated under reduced pressure to get crude 6 (60.2 g) as a brown solid. ESI-LCMS: m/z 844.2 [M+H]⁺.

Preparation of (7): To a solution of 6 (60.2 g) in DMF (500.0 mL), KOAc (36.1 g, 367.8 mmol) were added, The mixture was stirred at 25° C. for 14 h and 50° C. for 3 h, TLC showed 6 was consumed completely. Then filtered and the solution diluted with EA. The organic layer was washed with H₂O and brine. The solvent was concentrated under reduced pressure, residue was purified by c.c. (PE/EA=5:1˜1:1) to give 7 (28.0 g, 39.3 mmol, 53.5% yield) as yellow solid. ESI-LCMS: m/z 710.2 [M−H]⁻; ¹H-NMR (400 MHz, DMSO-d₆): δ 7.37-7.25 (m, 33H), 6.34-6.31 (m, 2H), 6.13-6.10 (m, 1H), 5.08 (d, J=4.2 Hz, 1H), 3.99 (d, J=7.6 Hz, 1H), 3.74 (s, 1H), 3.12 (t, J=9.2 Hz, 1H), 2.72-2.69 (m, 1H).

Preparation of (8): To a solution of 7 (28.0 g, 39.3 mmol) in DCM (300.0 mL), DAST (31.6 g, 196.6 mmol) was added at 0° C., the mixture was stirred at 25° C. for 16 h, TLC showed 7 was consumed completely. Then the solution was diluted with EA, washed with NaHCO₃ twice, the solvent was removed under reduced pressure, residue was purified by c.c. (PE/EA=5:1˜3:1) to give 8 (5.0 g, 7.0 mmol, 17.8% yield) as a white solid. ESI-LCMS: m/z 748.2 [M+2NH₄]⁺; ¹H-NMR (400 MHz, DMSO-d₆): δ 7.57-7.18 (m, 35H), 6.30 (d, J=8.8 Hz, 1H), 6.00 (d, J=19.5 Hz, 1H), 5.92-5.88 (m, 1H), 4.22-4.17 (m, 2H), 3.94 (s, 0.5H), 3.80 (s, 0.5H), 3.35-3.31 (m, 1H), 3.14-3.10 (m, 1H); ¹⁹F-NMR (376 MHz, DMSO-d₆): δ −193.54.

Preparation of (9): To a solution of 8 (5.0 g, 7.0 mmol) in DCM (60.0 mL) was added DCA (3.6 mL) and TES (15.0 mL). The mixture was stirred at 20° C. for 1 h, TLC showed 8 was consumed completely. Then the solution was concentrated under reduced pressure, the residue was purified by Flash-Prep-HPLC with the following conditions (IntelFlash-1): Column, C18 silica gel; mobile phase, CH₃CN/H₂O (0.5% NH₄HCO₃)=0/1 increasing to CH₃CN/H₂O (0.5% NH₄HCO₃)=1/3 within 25 min, the eluted product was collected at CH₃CN/H₂O (0.5% NH₄HCO₃)=0/1; Detector, UV 254 nm. This resulted in to give 9 (1.6 g, 6.9 mmol, 98.5% yield) as a white solid. ESI-LCMS: m/z 229.9 [M+H]⁺; ¹H-NMR (400 MHz, DMSO-d₆): δ 8.06-8.04 (m, 1H), 7.48-7.43 (m, 1H), 6.39 (d, J=9.0 Hz, 1H), 6.31-6.27 (m, 1H), 6.16-6.11 (m, 1H), 5.63 (s, 1H), 5.26 (s, 1H), 4.95-4.81 (m, 1H), 4.20-411 (m, 1H), 3.95 (d, J=8.2 Hz, 1H), 3.84 (d, J=12.4 Hz, 1H), 3.64 (d, J=12.1 Hz, 1H); ¹⁹F-NMR (376 MHz, DMSO-d₆): δ −201.00.

Preparation of (10): To a solution of 9 (1.6 g, 6.9 mmol) in pyridine (20.0 mL) was added DMTrCl (3.5 g, 10.5 mmol) at 20° C. and stirred for 1 h. TLC showed 9 was consumed completely. Water was added and extracted with EA, the organic layer was washed with NaHCO₃ and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=1/1; Detector, UV 254 nm. This resulted in to give 10 (2.2 g, 4.2 mmol, 60.8% yield) as a white solid. ESI-LCMS: m/z 530.1 [M−H]⁻; ¹H-NMR (400 MHz, DMSO-d₆): δ 7.93-7.91 (m, 1H), 7.47-7.23 (m, 1OH), 6.91-6.89 (m, 4H), 6.41 (d, J=8.8 Hz, 1H), 6.13 (d, J=18.8 Hz, 1H), 6.00-5.96 (m, 1H), 5.68 (d, J=6.6 Hz, 1H), 5.01 (d, J=4.2 Hz, 0.5H), 4.88 (d, J=4.2 Hz, 0.5H), 4.42-4.31 (m, 1H), 4.10-4.08 (m, 1H), 3.74 (s, 6H), 3.40-3.34 (m, 2H); ¹⁹F-NMR (376 MHz, DMSO-d₆): δ −199.49.

Preparation of Example 34 monomer: To a solution of 10 (2.2 g, 4.2 mmol) in DCM (20.0 mL) was added DCI (415 mg, 3.5 mmol) and CEP (1.5 g, 4.9 mmol) under N₂ pro. The mixture was stirred at 20° C. for 0.5 h. TLC showed 10 was consumed completely. The product was extracted with DCM, the organic layer was washed with H₂O and brine. Then the solution was concentrated under reduced pressure and the residue was purified by cc (PE/EA=5:1˜1:1) and Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give Example 34 monomer (2.1 g, 3.0 mmol, 73.1% yield) as a white solid. ESI-ESI-LCMS: m/z 732.2 [M+H]⁺; ¹H-NMR (400 MHz, DMSO-d₆): δ 7.98-7.92 (m, 1H), 7.42-7.24 (m, 1OH), 6.91-6.85 (m, 4H), 6.43-6.39 (m, 1H), 6.18-6.11 (m, 1H), 6.01-5.97 (m, 1H), 5.22-5.19 (m, 0.5H), 5.09-5.06 (m, 0.5H), 4.73-4.52 (m, 1H), 4.21-4.19 (m, 1H), 3.79-3.62 (m, 7H), 3.57-3.47 (m, 4H), 3.32-3.28 (m, 1H), 2.75-2.58 (m, 1H), 1.13-0.92 (m, 12H); ¹⁹F-NMR (376 MHz, DMSO-d₆): δ −196.82, −196.84, −197.86, −197.88; ³¹P-NMR (162 MHz, DMSO-d₆): δ 149.88, 149.83, 149.39, 149.35.

### Example 35. Synthesis of Monomer

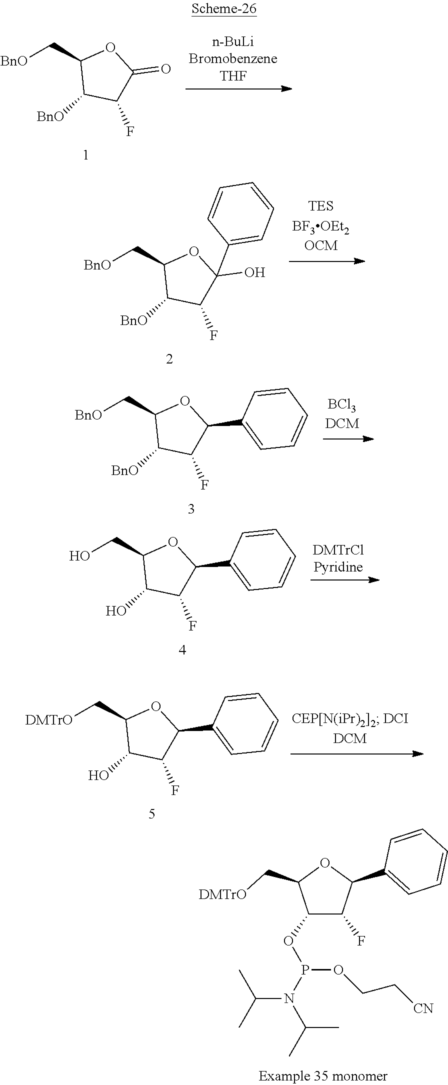

Preparation of (2): To the solution of Bromobenzene (2.1 g, 13.6 mmol) in dry THF (15 mL) was added 1.6 M n-BuLi (7 mL, 11.8 mmol) drop wise at −78° C. The mixture was stirred at −78° C. for 0.5 h. Then the 1 (3.0 g, 9.1 mmol, Wang, Guangyi et al, Journal of Medicinal Chemistry, 2016, 59(10), 4611-4624) was dissolved in THF (15 mL) and added to the mixture drop wise with keeping at −78° C. Then the reaction mixture was stirred at −78° C. for 1 hr. LC-MS showed 1 was consumed completely. Then the solution was added to saturated aq. NH₄C1 and the resulting mixture was extracted with EA. The combined organic layer was washed with water and brine, dried over Na₂SO₄, and concentrated under reduced pressure to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 2 (3.0 g, 7.3 mmol, 80.0%) as a white solid. ESI-LCMS: m/z 391 [M−OH]⁻.

Preparation of (3): To the solution of 2 (4.0 g, 9.8 mmol) in DCM (40 mL) was added TES (1.9 g, 11.7 mmol) at −78° C., and the mixture was added BF₃·OEt₂ (2.1 g, 14.7 mmol) drop wise at −78° C. The mixture was stirred at −40° C. for 1 hr. LC-MS showed 2 was consumed completely. Then the solution was added to saturated aq·NaHCO₃ and the resulting mixture was extracted with DCM. The combined organic layer was washed with water and brine, dried over Na₂SO₄, and concentrated under reduced pressure to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=7/3; Detector, UV 254 nm. This resulted in to give 3 (3.1 g, 5.3 mmol, 54.0%) as a water clear oil. ESI-LCMS: m/z 410 [M+H₂O]⁺; ¹H-NMR (400 MHz, CDCl₃: δ 7.48-7.25 (m, 15H), 5.24-5.13 (m, 1H), 4.93-4.74 (m, 1H), 4.74-4.46 (m, 4H), 4.37-4.25 (m, 1H), 4.19-4.05 (m, 1H), 4.00-3.80 (m, 1H), 3.77-3.63 (m, 1H). ¹⁹F-NMR (376 MHz, CDCl₃): δ −196.84.

Preparation of (4): To the solution of 3 (2.1 g, 5.3 mmol) in dry DCM (20 mL) was added 1 M BCl₃ (25 mL, 25.5 mmol) drop wise at −78° C., and the reaction mixture was stirred at −78° C. for 0.5 hr. LC-MS showed 3 was consumed completely. After completion of reaction, the resulting mixture was poured into water (50 mL). The solution was extracted with DCM and the combined organic layer was concentrated under reduced pressure to give a crude. The crude in MeOH (4 mL) was added 1 M NaOH (15 mL), and the mixture was stirred at r.t for 5˜10 min. The mixture was extracted with EA. The combined organic layer was washed with brine, dried over Na₂SO₄, and concentrated under reduced pressure to give a residue which was purified by silica gel column chromatography (eluent, DCM:MeOH=40:1˜15:1) to give 4 (1.0 g, 4.7 mmol, 88.6%) as a water clear oil. ESI-LCMS: m/z 211 [M−H]⁻; ¹H-NMR (400 MHz, DMSO-d₆): δ 7.58-7.19 (m, 5H), 5.41 (d, J=6.1 Hz, 1H), 5.09-5.95 (m, 1H), 5.95-4.84 (m, 1H), 4.82-4.59 (m, 1H), 4.14-3.94 (m, 1H), 3.89-3.80 (m, 1H), 3.78-3.67 (m, 1H), 3.65-3.53 (m, 1H). ¹⁹F-NMR (376 MHz, DMSO-d₆): δ −196.46.

Preparation of (5): To a solution of 4 (1.0 g, 4.7 mmol) in Pyridine (10 mL) was added DMTrCl (2.0 g, 5.7 mmol). The reaction mixture was stirred at r.t. for 2 hr. LCMS showed 4 was consumed and water (100 mL) was added. The product was extracted with EA (100 mL) and the organic layer was washed with brine and dried over Na₂SO₄ and concentrated to give the crude. The crude was further purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in to give 5 (2.1 g, 4.1 mmol, 87.0%) as a red oil. ESI-LCMS: m/z 513 [M−H]⁻; ¹H-NMR (400 MHz, DMSO-d₆): δ 7.56-7.16 (m, 14H), 6.94-9.80 (m, 4H), 5.45 (d, J=6.3 Hz, 1H), 5.21-5.09 (m, 1H), 4.89-4.68 (m, 1H), 4.18-4.03 (m, 2H), 3.74 (s, 6H), 3.33-3.29 (m, 1H), 3.26-3.17 (m, 1H). ¹⁹F-NMR (376 MHz, DMSO-d₆): δ −194.08.

Preparation of Example 35 monomer: To a suspension of 5 (2.1 g, 4.1 mmol) in DCM (20 mL) was added DCI (410 mg, 3.4 mmol) and CEP[N(iPr)₂]₂ (1.5 g, 4.9 mmol). The mixture was stirred at r.t. for 1 h. LC-MS showed 5 was consumed completely. The solution was washed with water twice and washed with brine and dried over Na₂SO₄. Then concentrated to give the crude. The crude was purification by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 35 monomer (2.1 g, 2.9 mmol, 70.0%) as a white solid. ESI-LCMS: m/z 715 [M+H]⁺; ¹H-NMR (400 MHz, DMSO-d₆): δ 7.59-7.16 (m, 14H), 6.94-9.80 (m, 4H), 5.26-5.12 (m, 1H), 5.06-4.77 (m, 1H), 4.50-4.20 (m, 1H), 4.20-4.10 (m, 1H), 3.83-3.63 (m, 7H), 3.59-3.37 (m, 4H), 3.25-3.13 (m, 1H), 2.80-2.66 (m, 1H), 2.63-2.53 (m, 1H), 1.18-0.78 (m, 12H). ¹⁹F-NMR (376 MHz, DMSO-d₆): δ −194.40, −194.42, −194.50, −194.53. ³¹P-NMR (162 MHz, DMSO-d₆): δ 149.38, 149.30, 149.02, 148.98.

### Example 36: Synthesis of 5′ End Cap Monomer

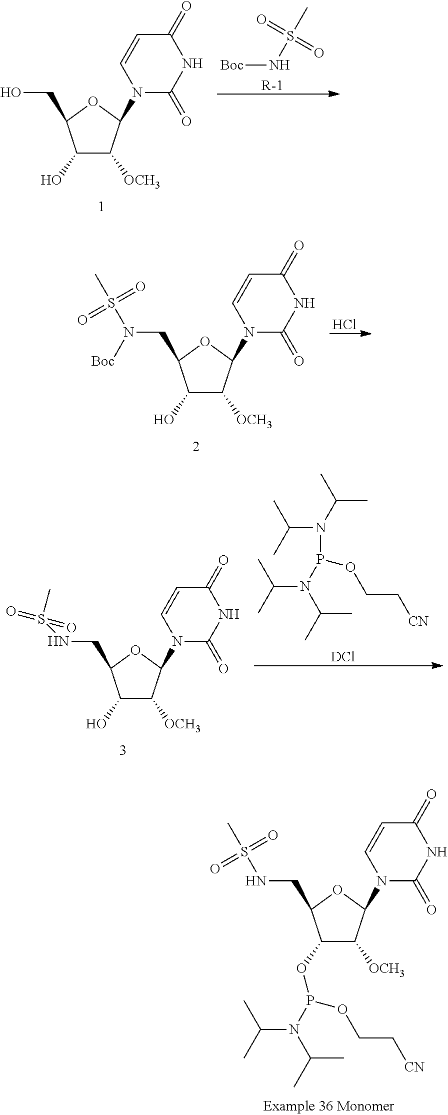

Preparation of (2): 1 (15 g, 58.09 mmol) and tert-butyl N-methylsulfonylcarbamate (17.01 g, 87.13 mmol) were dissolved in THF (250 mL), and PPh₃ (30.47 g, 116.18 mmol) was added followed by dropwise addition of DIAD (23.49 g, 116.18 mmol, 22.59 mL) at 0° C. The reaction mixture was stirred at 15° C. for 12 h. Upon completion as monitored by TLC (DCM/MeOH=10/1), the reaction mixture was evaporated to give a residue. The residue was purified by flash silica gel chromatography (ISCO@; 120 g SepaFlash@ Silica Flash Column, Eluent of 0˜20% MeOH/DCM gradient @60 mL/min) to give 2 (6.9 g, 24.28% yield) as a white solid. ESI-LCMS: m/z 457.9 [M+Na]⁺; ¹H NMR (400 MHz, CDCl₃) δ=8.64 (br s, 1H), 7.64 (d, J=8.2 Hz, 1H), 5.88 (d, J=1.9 Hz, 1H), 5.80 (dd, J=2.2, 8.2 Hz, 1H), 4.19-4.01 (m, 3H), 3.90 (dt, J=5.5, 8.2 Hz, 1H), 3.82-3.78 (m, 1H), 3.64 (s, 3H), 3.32 (s, 3H), 2.75 (d, J=8.9 Hz, 1H), 1.56 (s, 9H).

Preparation of (3): 2 (6.9 g, 15.85 mmol) was dissolved in MeOH (40 mL), and a solution of HCl/MeOH (4 M, 7.92 mL) was added dropwise. The reaction mixture was stirred at 15° C. for 12 h, and then evaporated to give a residue. The residue was purified by flash silica gel chromatography (ISCO®; 40 g SepaFlash® Silica Flash Column, Eluent of 0˜10% MeOH/DCM gradient @40 mL/min) to give 3 (2.7 g, 50.30% yield) as a white solid. ESI-LCMS: m/z 336.0 [M+H]⁺; ¹H NMR (400 MHz, CD₃CN) δ=9.20 (br s, 1H), 7.52 (d, J=8.1 Hz, 1H), 5.75 (d, J=3.8 Hz, 1H), 5.64 (dd, J=2.0, 8.1 Hz, 1H), 5.60-5.52 (m, 1H), 4.15-3.99 (m, 1H), 3.96-3.81 (m, 2H), 3.46 (s, 3H), 3.44-3.35 (m, 1H), 3.34-3.26 (m, 1H), 2.92 (s, 3H).

Preparation of (Example 36 monomer): To a solution of 3 (2.14 g, 6.38 mmol) in DCM (20 mL) was added dropwise 3-bis(diisopropylamino)phosphanyloxypropanenitrile (2.50 g, 8.30 mmol, 2.63 mL) at 0° C., followed by 1H-imidazole-4, 5-dicarbonitrile (829 mg, 7.02 mmol), and the mixture was purged under Ar for 3 times. The reaction mixture was stirred at 15° C. for 2 h. Upon completion, the mixture was quenched with 5% NaHCO₃ (20 mL), extracted with DCM (20 mL*2), washed with brine (15 mL), dried over Na₂SO₄, filtered, and evaporated to give a residue. The residue was purified by flash silica gel chromatography (ISCO@; 40 g SepaFlash® Silica Flash Column, Eluent of 0˜10% (Phase B: i-PrOH/DCM=1/2)/Phase A: DCM with 5% TEA gradient @40 mL/min) to give Example 36 monomer (1.73 g, 48.59% yield) as a white solid. ESI-LCMS: m/z 536.3 [M+H]⁺; ¹H NMR (400 MHz, CD₃CN) δ=7.58-7.48 (m, 1H), 5.83-5.78 (m, 1H), 5.71-5.64 (m, 1H), 4.40-4.29 (m, 1H), 4.19-4.07 (m, 1H), 3.98 (td, J=5.3, 13.3 Hz, 1H), 3.90-3.78 (m, 2H), 3.73-3.59 (m, 3H), 3.41 (d, J=14.8 Hz, 4H), 2.92 (br d, J=7.0 Hz, 3H), 2.73-2.63 (m, 2H), 1.23-1.11 (m, 12H); ³¹P NMR (162 MHz, CD₃CN) δ=149.81, 150.37.

### Example 37: Synthesis of 5′ End Cap Monomer

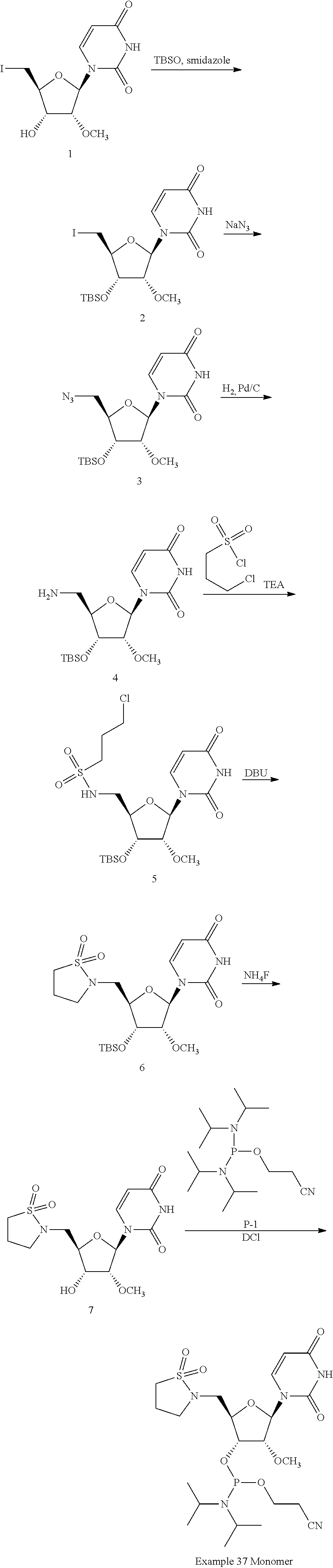

Preparation of (2): To a solution of 1 (10 g, 27.16 mmol) in DMF (23 mL) were added imidazole (3.70 g, 54.33 mmol) and TBSCl (8.19 g, 54.33 mmol) at 25° C. The mixture was stirred at 25° C. for 2 hr. Upon completion, the reaction mixture was diluted with H₂O (20 mL) and extracted with EA (30 mL*2). The combined organic layers were washed with brine (20 mL*2), dried over Na₂SO₄, filtered and concentrated under reduced pressure to give 2 (13 g, 99.2% yield) as a white solid. ESI-LCMS: m/z 482.9 [M+H]⁺.

Preparation of (3): To a solution of 2 (35.00 g, 72.56 mmol) in DMF (200 mL) was added NaN₃ (14.15 g, 217.67 mmol). The mixture was stirred at 60° C. for 17 h. Upon completion, the reaction mixture was diluted with H₂O (200 mL) and extracted with EA (200 mL*2). The combined organic layers were washed with brine (100 mL*2), dried over Na₂SO₄, filtered and concentrated under reduced pressure to give 3 (31.8 g, crude) as a yellow solid. ESI-LCMS: m/z 398.1 [M+H]⁺; ¹H NMR (400 MHz, DMSO-d₆) δ=11.21 (d, J=1.3 Hz, 1H), 7.50 (d, J=8.1 Hz, 1H), 5.57 (d, J=4.5 Hz, 1H), 5.46 (dd, J=2.1, 8.0 Hz, 1H), 4.06 (t, J=5.2 Hz, 1H), 3.81-3.64 (m, 2H), 3.44-3.30 (m, 2H), 2.31-2.25 (m, 3H), 0.65 (s, 9H), −0.13 (s, 6H).

Preparation of (4): To a solution of 3 (7 g, 17.61 mmol) in THF (60 mL) was added Pd/C (2 g) at 25° C. The reaction mixture was stirred at 25° C. for 3 h under H₂ atmosphere (15 PSI). The reaction mixture was filtered, and the filtrate was concentrated to give 4 (5.4 g, 75.11% yield) as a gray solid. ESI-LCMS: m/z 372.1 [M+H]; ¹H NMR (400 MHz, DMSO-d₆) δ=7.93 (d, J=8.0 Hz, 1H), 5.81 (d, J=5.5 Hz, 1H), 5.65 (d, J=8.3 Hz, 1H), 4.28 (t, J=4.6 Hz, 1H), 3.88 (t, J=5.3 Hz, 1H), 3.74 (q, J=4.6 Hz, 1H), 3.31 (s, 3H), 2.83-2.66 (m, 2H), 0.88 (s, 9H), 0.09 (s, 6H).

Preparation of (5): To a solution of 4 (3 g, 8.08 mmol) in DCM (30 mL) was added TEA (2.45 g, 24.23 mmol, 3.37 mL) followed by dropwise addition of 3-chloropropane-1-sulfonyl chloride (1.50 g, 8.48 mmol, 1.03 mL) at 25° C. The reaction mixture was stirred at 25° C. for 18 h under N₂ atmosphere. Upon completion, the reaction mixture was diluted with H₂O (50 mL) and extracted with DCM (50 mL*2). The combined organic layers were washed with brine (50 mL*2), dried over Na₂SO₄, filtered and concentrated under reduced pressure. The residue was purified by flash silica gel chromatography (ISCO®; 24 g SepaFlash® Silica Flash Column, Eluent of 0˜30% MeOH/DCM @50 mL/min) to give 5 (3.6 g, 84.44% yield) as a white solid. ESI-LCMS: m/z 512.1 [M+H]; ¹H NMR (400 MHz, DMSO-d₆) δ=11.42 (s, 1H), 7.75 (d, J=8.1 Hz, 1H), 7.49 (t, J=6.2 Hz, 1H), 5.83 (d, J=5.8 Hz, 1H), 5.70-5.61 (m, 1H), 4.33-4.23 (m, 1H), 3.95 (t, J=5.5 Hz, 1H), 3.90-3.78 (m, 1H), 3.73 (t, J=6.5 Hz, 2H), 3.30 (s, 3H), 3.26-3.12 (m, 4H), 2.14-2.02 (m, 2H), 0.88 (s, 9H), 0.11 (d, J=3.3 Hz, 6H).

Preparation of (6): To a solution of 5 (5 g, 9.76 mmol) in DMF (45 mL) was added DBU (7.43 g, 48.82 mmol, 7.36 mL). The mixture was stirred at 25° C. for 16 h. The reaction mixture was concentrated to give a residue, diluted with H₂O (50 mL) and extracted with EA (50 mL*2). The combined organic layers were washed with brine (50 mL*2), dried over Na₂SO₄, filtered and concentrated under reduced pressure. The residue was purified by flash silica gel chromatography (ISCO®; 24 g SepaFlash® Silica Flash Column, Eluent of 0˜80% EA/PE @40 mL/min) to give 6 (4.4 g, 89.06% yield) as a white solid. ESI-LCMS: m/z 476.1 [M+H]⁺; ¹H NMR (400 MHz, DMSO-d₆) δ=11.43 (d, J=1.7 Hz, 1H), 7.72 (d, J=8.1 Hz, 1H), 5.82 (d, J=4.8 Hz, 1H), 5.67 (dd, J=2.1, 8.1 Hz, 1H), 4.22 (t, J=5.1 Hz, 1H), 3.99-3.87 (m, 2H), 3.33-3.27 (m, 6H), 3.09 (dd, J=6.6, 14.7 Hz, 1H), 2.26-2.16 (m, 2H), 0.88 (s, 9H), 0.10 (d, J=3.8 Hz, 6H).

Preparation of (7): To a solution of 6 (200 mg, 420.49 umol) in MeOH (2 mL) was added NH₄F (311.48 mg, 8.41 mmol, 20 eq), and the mixture was stirred at 80° C. for 2 h. The mixture was filtered and concentrated to give a residue, which was purified by flash silica gel chromatography (ISCO®; 4 g SepaFlash® Silica Flash Column, Eluent of 0˜50% MeOH/DCM @20 mL/min) to give 7 (120 mg, 76.60% yield) as a white solid. ESI-LCMS: m/z 362.1 [M+H]⁺; ¹H NMR (400 MHz, DMSO-d₆) δ=11.37 (br s, 1H), 7.68 (d, J=8.1 Hz, 1H), 5.81 (d, J=4.6 Hz, 1H), 5.65 (d, J=8.0 Hz, 1H), 4.02 (q, J=5.6 Hz, 1H), 3.95-3.83 (m, 2H), 3.34 (s, 9H), 3.09 (dd, J=6.9, 14.6 Hz, 1H), 2.26-2.14 (m, 2H).

Preparation of (Example 37 monomer): To a solution of 7 (1.5 g, 4.15 mmol) in CH₃CN (12 mL) were added 3-bis(diisopropylamino)phosphanyloxypropanenitrile (1.63 g, 5.40 mmol, 1.71 mL) and 1H-imidazole-4,5-dicarbonitrile (539.22 mg, 4.57 mmol) in one portion at 0° C. The reaction mixture was gradually warmed to 25° C. The reaction mixture was stirred at 25° C. for 2 h under N₂ atmosphere. Upon completion, the reaction mixture was diluted with NaHCO₃ (20 mL) and extracted with DCM (20 mL*2). The combined organic layers were washed with brine (20 mL*2), dried over Na₂SO₄, filtered and concentrated under reduced pressure to give a residue, which was purified by flash silica gel chromatography (ISCO®; 12 g SepaFlash® Silica Flash Column, Eluent of 0˜85% EA/PE with 0.5% TEA @30 mL/min to give Example 37 monomer (800 mg, 33.6% yield,) as a white solid. ESI-LCMS: m/z 562.3 [M+H]; ¹H NMR (400 MHz, CD₃CN) δ=9.28 (br s, 1H), 7.55 (br dd, J=8.3, 12.8 Hz, 1H), 5.86 (br d, J=3.9 Hz, 1H), 5.65 (br d, J=8.0 Hz, 1H), 4.33-4.06 (m, 2H), 4.00-3.89 (m, 1H), 4.08-3.86 (m, 1H), 3.89-3.72 (m, 4H), 3.43 (br d, J=15.1 Hz, 6H), 3.23-3.05 (m, 3H), 2.69 (br s, 2H), 2.36-2.24 (m, 2H), 1.26-1.10 (m, 12H); ³¹P NMR (162 MHz, CD₃CN) δ=149.94, 149.88.

### Example 38: Synthesis of 5′ End Cap Monomer

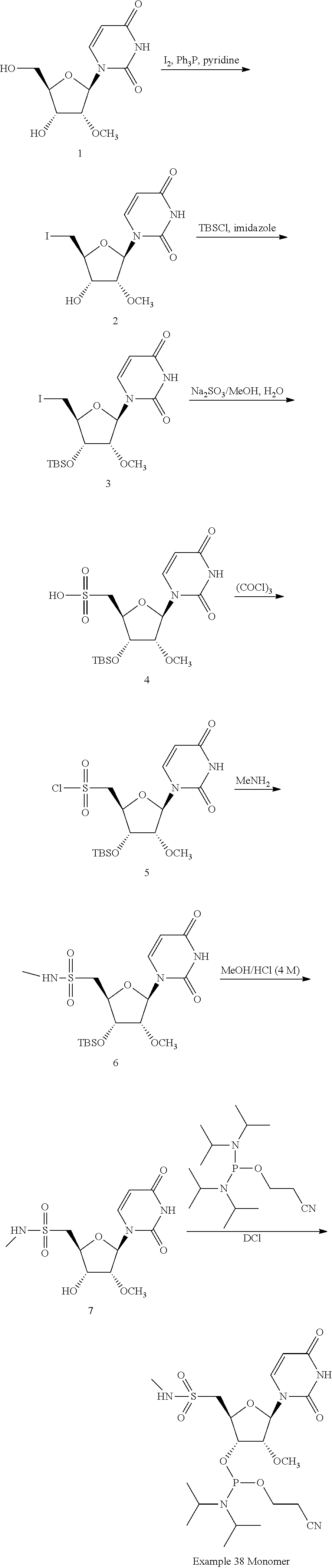

Preparation of (2): To a solution of 1 (30 g, 101.07 mmol, 87% purity) in CH₃CN (1.2 L) and Py (60 mL) were added I2 (33.35 g, 131.40 mmol, 26.47 mL) and PPh₃ (37.11 g, 141.50 mmol) in one portion at 10° C. The reaction was stirred at 25° C. for another 48 h. The mixture was diluted with aq·Na₂S₂O₃ (300 mL) and aq·NaHCO₃ (300 mL), concentrated to remove CH₃CN, and then extracted with EtOAc (300 mL*3). The combined organic layers were washed with brine (300 mL), dried over Na₂SO₄, filtered and concentrated under reduced pressure to give a residue. The residue was purified by flash silica gel chromatography (ISCO®; 330 g SepaFlash® Silica Flash Column, Eluent of 0˜60% Methanol/Dichloromethane gradient @ 100 mL/min) to give 2 (28.2 g, 72.00% yield, 95% purity) as a brown solid. ESI-LCMS: m/z 369.1 [M+H]⁺: 1H NMR (400 MHZ, DMSO-d₆) δ=11.43 (s, 1H), 7.68 (d, J-8.1 Hz, 1H), 5.86 (d, J=5.5 Hz, 1H), 5.69 (d, J=8.1 Hz, 1H), 5.46 (d, J=6.0 Hz, 1H), 4.08-3.96 (m, 2H), 3.90-3.81 (m, 1H), 3.60-3.51 (m, 1H), 3.40 (dd, J-6.9, 10.6 Hz, 1H), 3.34 (s, 3H).

Preparation of (3): To a solution of 2 in DMF (90 mL) were added imidazole (4.25 g, 62.48 mmol) and TBSCl (6.96 g, 46.18 mmol) in one portion at 15° C. The mixture was stirred at 15° C. for 6 h. The reaction mixture was quenched by addition of H₂O (300 mL) and extracted with EtOAc (300 mL*2). The combined organic layers were washed with brine (300 mL), dried over Na₂SO₄, filtered and concentrated under reduced pressure to give 3 (13.10 g, crude) as a white solid. ESI-LCMS: m/z 483.0 [M+H]⁺.

Preparation of (4): To a solution of 3 (10 g, 20.73 mmol) in MeOH (20 mL), H₂O (80 mL), and dioxane (20 mL) was added Na₂SO₃ (15.68 g, 124.38 mmol), and the mixture was stirred at 80° C. for 24 h. The reaction mixture was concentrated under reduced pressure to remove MeOH. The aqueous layer was extracted with EtOAc (80 mL*2) and concentrated under reduced pressure to give a residue. The residue was triturated with MeOH (100*3 mL) to give 4 (9.5 g, 94.48% yield, 90% purity) as a white solid. ESI-LCMS: m/z 437.0 [M+H]⁺.

Preparation of (5): To a solution of 4 (11 g, 21.42 mmol, 85% purity) in DCM (120 mL) was added DMF (469.65 mg, 6.43 mmol, 494.37 uL) at 0° C., followed by dropwise addition of oxalyl dichloride (13.59 g, 107.10 mmol, 9.37 mL). The mixture was stirred at 20° C. for 2 h. The reaction mixture was quenched by addition of water (60 mL) and the organic layer 5 (0.1125 M, 240 mL DCM) was used directly for next step. (This reaction was set up for two batches and combined) ESI-LCMS: m/z 455.0 [M+H]⁺.

Preparation of (6): 5 (186.4 mL, 0.1125 M in DCM) was diluted with DCM (60 mL) and treated with methylamine (3.26 g, 41.93 mmol, 40% purity). The mixture was stirred at 20° C. for 2 h. The reaction mixture was concentrated under reduced pressure to give a residue. The residue was purified by flash silica gel chromatography (ISCO®; 40 g SepaFlash® Silica Flash Column, Eluent of 0˜10%, MeOH/DCM gradient @40 mL/min) to give AGS-9-3-008 (1.82 g, 18.53% yield, 96% purity) as a yellow solid. ESI-LCMS: m/z 472.0 [M+Na]; ¹H NMR (400 MHz, CDCl₃) δ=9.08 (s, 1H), 7.31 (d, J=8.1 Hz, 1H), 5.78 (d, J=8.1 Hz, 1H), 5.57 (d, J=3.8 Hz, 1H), 4.61-4.48 (m, 1H), 4.41-4.27 (m, 2H), 4.13-4.03 (m, 1H), 3.46 (s, 3H), 3.43-3.33 (m, 2H), 2.78 (d, J=5.2 Hz, 3H), 0.92 (s, 9H), 0.13 (s, 6H).

Preparation of (7): To a solution of 6 (2.3 g, 5.12 mmol) in MeOH (12 mL) was added HCl/MeOH (4 M, 6.39 mL). The mixture was stirred at 20° C. for 2 h. The reaction mixture was concentrated under reduced pressure to give a residue. The residue was purified by flash silica gel chromatography (ISCO®; 24 g SepaFlash® Silica Flash Column, Eluent of 0-15%, MeOH/DCM gradient @30 mL/min) to give 7 (1.4 g, 79.98% yield) as a pink solid. ESI-LCMS: m/z 336.1 [M+H]⁺; ¹H NMR (400 MHz, CDCl₃) δ=9.12 (s, 1H), 7.39 (d, J=8.0 Hz, 1H), 5.79 (d, J=3.3 Hz, 1H), 5.66 (dd, J=2.1, 8.2 Hz, 1H), 5.13 (s, 1H), 4.13 (t, J=4.0, 7.4 Hz, 1H), 4.07-4.02 (m, 1H), 3.87 (dd, J=3.3, 5.5 Hz, 1H), 3.47 (s, 3H), 3.43-3.37 (m, 2H), 2.65 (d, J=4.5 Hz, 3H).

Preparation of (Example 38 monomer): To a mixture of 7 (1.7 g, 5.07 mmol) and 4A MS (1.4 g) in MeCN (18 mL) was added 3-bis(diisopropylamino)phosphanyloxypropanenitrile (1.99 g, 6.59 mmol, 2.09 mL) at 0° C., followed by addition of 1H-imidazole-4,5-dicarbonitrile (658.57 mg, 5.58 mmol) in one portion at 0° C. The mixture was stirred at 20° C. for 2 h. Upon completion, the reaction mixture was quenched by addition of sat. NaHCO₃ solution (20 mL) and diluted with DCM (40 mL). The organic layer was washed with sat. NaHCO₃ (20 mL*2), dried over Na₂SO₄, filtered and concentrated under reduced pressure to give a residue. The residue was purified by a flash silica gel column (0% to 5% i-PrOH in DCM with 5% TEA) to give Example 38 monomer (1.30 g, 46.68% yield) as a white solid. ESI-LCMS: m/z 536.2 [M+H]⁺; ¹H NMR (400 MHz, CD₃CN) δ=9.00 (s, 1H), 7.40 (d, J=8.0 Hz, 1H), 5.85-5.76 (m, 1H), 5.64 (d, J=8.0 Hz, 1H), 5.08 (d, J=5.0 Hz, 1H), 4.42-4.21 (m, 2H), 4.00 (td, J=4.6, 9.3 Hz, 1H), 3.89-3.61 (m, 4H), 3.47-3.40 (m, 4H), 3.37-3.22 (m, 1H), 2.71-2.60 (m, 5H), 1.21-1.16 (m, 11H), 1.21-1.16 (m, 1H); ³¹P NMR (162 MHz, CD₃CN) δ=150.07, 149.97

### Example 39: Synthesis of 5′ End Cap Monomer

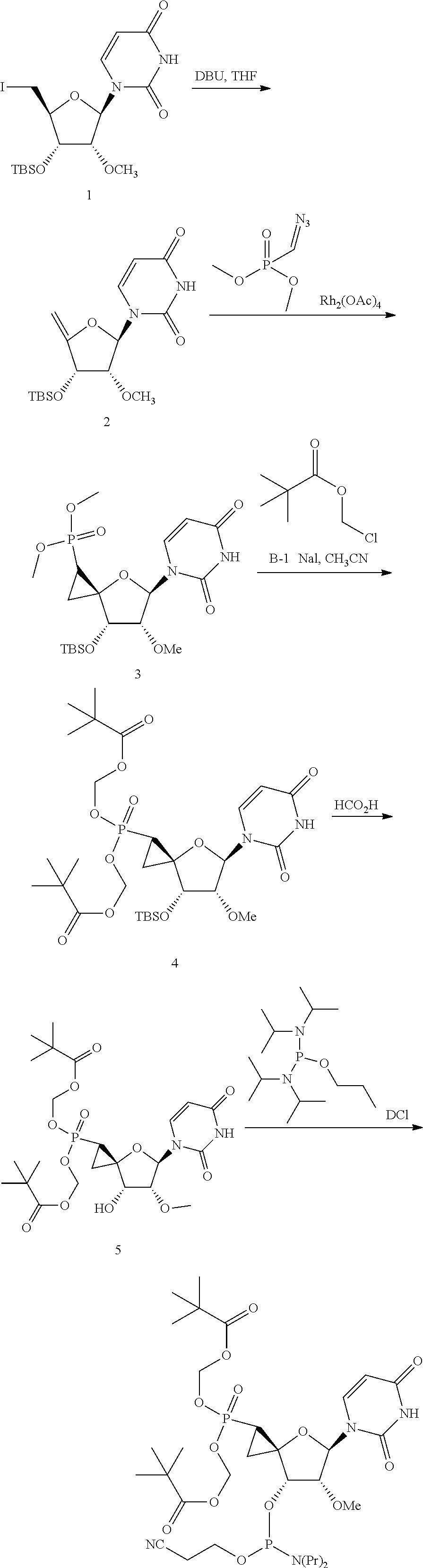

Preparation of (2): To a solution of 1 (13.10 g, 27.16 mmol) in THF (100 mL) was added DBU (20.67 g, 135.78 mmol, 20.47 mL). The mixture was stirred at 60° C. for 6 h. Upon completion, the reaction mixture was quenched by addition of sat·NH₄C1 solution (600 mL) and extracted with EA (600 mL*2). The combined organic layers were washed with brine (100 ml), dried over Na₂SO₄, filtered and concentrated under reduced pressure to give a residue. The residue was purified by flash silica gel chromatography (ISCO®; 120 g SepaFlash® Silica Flash Column, Eluent of 0˜50% (Phase B: ethyl acetate:dichloromethane=1:1)/Phase A: petroleum ethergradient@45 mL/min) to give 2 (5.9 g, 60.1% yield,) as a white solid. ESI-LCMS: m/z 355.1 [M+H]⁺; ¹H NMR (400 MHz, DMSO-d₆) δ=11.61-11.30 (m, 1H), 7.76-7.51 (m, 1H), 6.04 (d, J=5.4 Hz, 1H), 5.75 (s, 1H), 5.73-5.67 (m, 1H), 4.78 (d, J=4.9 Hz, 1H), 4.41 (d, J=1.1 Hz, 1H), 4.30 (t, J=4.8 Hz, 1H), 4.22 (d, J=1.4 Hz, 1H), 4.13 (t, J=5.1 Hz, 1H), 4.06-3.97 (m, 1H), 3.94-3.89 (m, 1H), 3.82-3.75 (m, 1H), 3.33 (s, 3H), 3.30 (s, 2H), 1.17 (t, J=7.2 Hz, 1H), 0.89 (s, 9H), 0.16-0.09 (m, 6H).

Preparation of (3): To a solution of 2 (4 g, 11.28 mmol) in DCM (40 mL) was added Ru(II)-Pheox (214.12 mg, 338.53 umol) in one portion followed by addition of diazo(dimethoxyphosphoryl)methane (2.54 g, 16.93 mmol) dropwise at 0° C. under N₂. The reaction was stirred at 20° C. for 16 h. Upon completion, the reaction mixture was filtered and concentrated under reduced pressure to give a residue. The residue was purified by flash silica gel chromatography (ISCO®; 80 g SepaFlash® Silica Flash Column, Eluent of 0˜4% MeOH/DCM@60 mL/min) to give 3 (5 g, 86.47% yield) as a red liquid. ESI-LCMS: m/z 477.1 [M+H]⁺; ¹H NMR (400 MHz, DMSO-d₆) δ=11.46 (s, 1H), 7.49 (d, J=8.0 Hz, 1H), 6.01-5.87 (m, 1H), 5.75 (dd, J=2.0, 8.0 Hz, 1H), 4.58 (d, J=3.8 Hz, 1H), 4.23 (dd, J=3.8, 7.8 Hz, 1H), 3.80-3.68 (m, 6H), 3.30 (s, 3H), 1.65-1.46 (m, 2H), 1.28-1.16 (m, 1H), 0.91 (s, 9H), 0.10 (d, J=4.3 Hz, 6H); ³¹P NMR (162 MHz, DMSO-d₆) δ=27.5

Preparation of (4): To a mixture of 3 (2.8 g, 5.88 mmol) and NaI (1.76 g, 11.75 mmol) in CH₃CN (30 mL) was added chloromethyl 2,2-dimethylpropanoate (2.21 g, 14.69 mmol, 2.13 mL) at 25° C. The mixture was stirred at 80° C. for 40 h under Ar. The reaction mixture was filtered and concentrated under reduced pressure to give a residue. The residue was purified by flash silica gel chromatography (ISCO®; 40 g SepaFlash® Silica Flash Column, Eluent of 0˜50% Ethylacetate/Petroleum ether gradient @40 mL/min) to give 4 (2.1 g, 51.23% yield, 97% purity) as a yellow solid. ESI-LCMS: 677.3 [M+H]⁺.

Preparation of (5): A mixture of 4 (2.09 g, 3.09 mmol) in H₂O (1.5 mL) and HCOOH (741.81 mg, 15.44 mmol, 6 mL) was stirred at 15° C. for 40 h. Upon completion, the reaction mixture was quenched by saturated aq·NaHCO₃ (300 mL) and extracted with EA (300 mL*2). The combined organic layers were washed with brine (300 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜5% Methanol/Dichloromethane@45 mL/min) to give 5 (1.51 g, 85.19% yield) as a yellow solid. ESI-LCMS: 585.1 [M+Na]⁺; ¹H NMR (400 MHz, DMSO-d₆) δ=11.45 (d, J=1.8 Hz, 1H), 7.44 (d, J=8.2 Hz, 1H), 6.04 (d, J=7.5 Hz, 1H), 5.78-5.51 (m, 6H), 4.39 (t, J=4.4 Hz, 1H), 4.15 (dd, J=4.3, 7.4 Hz, 1H), 4.03 (q, J=7.1 Hz, 1H), 1.99 (s, 1H), 1.66 (dd, J=8.6, 10.8 Hz, 1H), 1.55-1.29 (m, 2H), 1.18 (d, J=2.0 Hz, 18H).

Preparation of (Example 39 monomer): To a solution of 5 (2.5 g, 4.44 mmol) in MeCN (30 mL) was added 3-bis(diisopropylamino)phosphanyloxypropanenitrile (1.74 g, 5.78 mmol, 1.84 mL) at 0° C., followed by 1H-imidazole-4,5-dicarbonitrile (577.36 mg, 4.89 mmol) in one portion under Ar. The mixture was gradually warmed to 20° C. and stirred at 20° C. for 1 h. The reaction mixture was quenched by addition of sat·NaHCO₃ solution (50 mL) and diluted with DCM (250 mL). The organic layer was washed with sat·NaHCO₃ solution (50 mL*2), dried over Na₂SO₄, filtered and concentrated under reduced pressure to give a residue. The residue was purified by a flash silica gel column (0% to 50% EA/PE with 0.5% TEA) to give Example 39 monomer (1.85 g, 54.1% yield) as a white solid. ESI-LCMS: 785.2 [M+Na]⁺; ¹H NMR (400 MHz, CD₃CN) δ=9.18 (s, 1H), 7.31 (d, J=8.3 Hz, 1H), 6.06 (d, J=7.8 Hz, 1H), 5.72-5.60 (m, 5H), 4.85-4.76 (m, 1H), 4.27 (m, 1H), 3.93-3.64 (m, 4H), 3.41 (d, J=16.6 Hz, 3H), 2.80-2.62 (m, 2H), 1.76-1.49 (m, 3H), 1.23-1.19 (m, 30H); ³¹P NMR (162 MHz, CD₃CN) δ=150.66 (s), 150.30, 24.77, 24.66.

### Example 40: Synthesis of 5′ End Cap Monomer

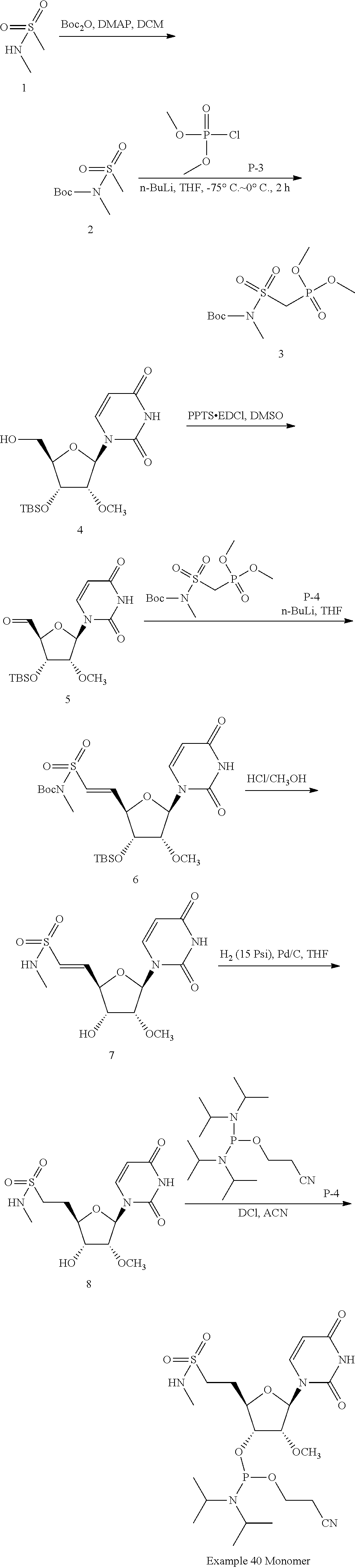

### Example 40 Monomer

Preparation of (2): To a solution of t (15 g, 137.43 mmol) in DCM (75 mL) were added Boc₂O (31.49 g, 144.30 mmol, 33.15 mL) and DMAP (839.47 mg, 6.87 mmol, 0.05 eq) at 0° C. The mixture was stirred at 20° C. for 16 hr, and concentrated under reduced pressure to give 2 (29.9 g, crude) as a yellow oil. ¹H NM/IR (400 MHz, CDCl₃) δ=3.23 (s, 3H), 3.16 (s, 3H), 1.51 (s, 9H).

Preparation of (3): To a solution of 2 (24.9 g, 118.99 mmol) in THF (250 mL) was added n-BuLi (2.5 M, 47.60 mL) dropwise at −78° C. under Ar and stirred at −78° C. for 1 hr. P-3 (17.19 g, 118.99 mmol, 12.83 mL) was added at 0° C. and stirred for 1 hr. The reaction mixture was quenched by saturated aq. NH₄C1 (100 mL), and then extracted with EA (100 mL*2). The combined organic layers were washed with brine (100 mL*2), dried over Na₂SO₄, filtered and concentrated under reduced pressure to give a residue. The residue was purified by flash silica gel chromatography (ISCO®; 80 g SepaFlash® Silica Flash Column, Eluent of 0˜₅₀% Ethylacetate/Petroleum ethergradient @65 mL/min) to give 3 (7.1 g, 18.62% yield) as a yellow oil. ESI-LCMS: 339.9 [M+Na]; ¹H NMR (400 MHz, CDCl₃) δ=4.12 (s, 1H), 4.08 (s, 1H), 3.83 (s, 3H), 3.81 (s, 3H), 3.22 (s, 3H), 1.51 (s, 9H).

Preparation of (5): To a mixture of 4 (15 g, 40.27 mmol) and PPTS (10.12 g, 40.27 mmol) in DMSO (75 mL) was added EDCI (23.16 g, 120.81 mmol) at 20° C. The mixture was stirred at 20° C. for 4 hr. The reaction mixture was diluted with water (150 mL) and extracted with EA (150 mL*2). The combined organic layers were washed with brine (150 mL*2), dried over Na₂SO₄, filtered and concentrated under reduced pressure to give 5 (12 g, crude) as a white solid. ESI-LCMS: 371.2[M+H]⁺; ¹H NMR (400 MHz, CDCl₃) δ=9.77 (s, 1H), 7.62 (d, J=8.1 Hz, 1H), 5.83-5.76 (m, 2H), 4.53 (d, J=4.3 Hz, 1H), 4.43 (br t, J=4.4 Hz, 1H), 3.95 (br t, J=4.7 Hz, 1H), 3.47-3.35 (m, 5H), 0.92 (s, 9H), 0.13 (d, J=5.8 Hz, 6H).

Preparation of (6): To a solution of P4 (8.02 g, 25.27 mmol) in THF (40 mL) was added n-BuLi (2.5 M, 8.42 mL) dropwise under Ar at −78° C., and the mixture was stirred at −78° C. for 0.5 hr. A solution of 4 (7.8 g, 21.05 mmol) in THF (40 mL) was added dropwise. The mixture was allowed to warm to 0° C. and stirred for another 2 hr. The reaction mixture was quenched by saturated aq. NH₄C1 solution (80 mL) and extracted with EA (80 mL). The combined organic layers were washed with brine (80 mL*2), dried over Na₂SO₄, filtered and concentrated under reduced pressure to give a residue. The residue was purified by flash silica gel chromatography (ISCO®; 80 g SepaFlash® Silica Flash Column, Eluent of 0˜38% ethylacetate/petroleum ether gradient @60 mL/min) to give 7 (7.7 g, 13.43 mmol, 63.8% yield) as a white solid. ESI-LCMS: 506.2 [M-tBu]⁺; ¹H NMR (400 MHz, CDCl₃) δ=8.97 (s, 1H), 7.25 (d, J=8.3 Hz, 1H), 6.95-6.88 (m, 1H), 6.87-6.81 (m, 1H), 5.83-5.77 (m, 2H), 4.58 (dd, J=4.4, 6.7 Hz, 1H), 4.05 (dd, J=5.0, 7.5 Hz, 1H), 3.82-3.77 (m, 1H), 3.53 (s, 3H), 3.20 (s, 3H), 1.50 (s, 9H), 0.91 (s, 9H), 0.11 (d, J=2.5 Hz, 6H).

Preparation of (7): To a solution of 6 (7.7 g, 13.71 mmol) in MeOH (10 mL) was added HCl/MeOH (4 M, 51.40 mL) at 20° C. The mixture was stirred at 20° C. for 16 hr. Upon completion, the reaction mixture was concentrated under reduced pressure to remove MeOH. The residue was purified by flash silica gel chromatography (ISCO®; 80 g SepaFlash® Silica Flash Column, Eluent of 0˜4% MeOH/DCM @60 mL/min) to give 7 (4.1 g, 86.11% yield) as a white solid. ESI-LCMS: 369.9 [M+Na]⁺; ¹H NMR (400 MHz, DMSO-d₆) δ=11.44 (s, 1H), 7.66 (d, J=8.3 Hz, 1H), 7.11 (q, J=4.9 Hz, 1H), 6.69 (dd, J=6.0, 15.1 Hz, 1H), 6.56-6.47 (m, 1H), 5.82 (d, J=4.0 Hz, 1H), 5.67 (dd, J=2.0, 8.0 Hz, 1H), 5.56 (br s, 1H), 4.42 (t, J=6.1 Hz, 1H), 4.13 (t, J=5.8 Hz, 1H), 3.97 (t, J=4.8 Hz, 1H), 3.39 (s, 3H), 2.48 (d, J=5.3 Hz, 3H)

Preparation of (8): To a solution of 7 (2.5 g, 7.20 mmol) in THF (25 mL) was added Pd/C (2.5 g, 10% purity) under H₂ atmosphere, and the suspension was degassed and purged with H₂ for 3 times. The mixture was stirred under H₂ (15 Psi) at 20° C. for 1 hr. Upon completion, the reaction mixture was filtered and concentrated under reduced pressure to give a residue. The residue was purified by flash silica gel chromatography (ISCO®; 25 g SepaFlash® Silica Flash Column, Eluent of 0˜5% Ethylacetate/Petroleum ethergradient @50 mL/min) to give 8 (2.2 g, 87.49% yield,) as a white solid. ESI-LCMS: 372.1 [M+Na]⁺; ¹H NMR (400 MHz, DMSO-d₆) δ=11.40 (s, 1H), 7.62 (d, J=8.0 Hz, 1H), 6.93 (q, J=4.9 Hz, 1H), 5.76 (d, J=4.5 Hz, 1H), 5.66 (d, J=8.0 Hz, 1H), 5.26 (d, J=6.3 Hz, 1H), 3.97 (q, J=5.9 Hz, 1H), 3.91-3.79 (m, 2H), 3.36 (s, 3H), 3.14-3.00 (m, 2H), 2.56 (d, J=5.0 Hz, 3H), 2.07-1.87 (m, 2H).

Preparation of (Example 40 monomer): To a solution of 8 (2.2 g, 6.30 mmol, 1 eq) in CH₃CN (25 mL) was added P-1 (2.47 g, 8.19 mmol, 2.60 mL, 1.3 eq) at 0° C., and then 1H-imidazole-4,5-dicarbonitrile (818.07 mg, 6.93 mmol, 1.1 eq) was added in one portion at 0° C. under Ar. The mixture was stirred at 20° C. for 2 hr. Upon completion, the reaction mixture was quenched by saturated aq·NaHCO₃ (25 mL), and extracted with DCM (25 mL*2). The combined organic layers were washed with brine (25 mL*2), dried over Na₂SO₄, filtered and concentrated under reduced pressure to give a residue. The residue was purified by flash silica gel chromatography (ISCO®; 40 g SepaFlash® Silica Flash Column, Eluent of 40˜85% ethylacetate/petroleum ether gradient @40 mL/min) to give Example 40 monomer (2.15 g, 61.32% yield) as a white solid. ESI-LCMS: 572.2 [M+Na]⁺; ¹H NMR (400 MHz, CD₃CN) δ=9.32 (br s, 1H), 7.39 (d, J=8.1 Hz, 1H), 5.82-5.75 (m, 1H), 5.66 (dd, J=0.7, 8.1 Hz, 1H), 5.14 (qd, J=4.9, 9.4 Hz, 1H), 4.24-4.02 (m, 2H), 3.99-3.93 (m, 1H), 3.90-3.60 (m, 4H), 3.43 (d, J=17.5 Hz, 3H), 3.18-3.08 (m, 2H), 2.74-2.61 (m, 5H), 2.19-2.11 (m, 1H), 2.09-1.98 (m, 1H), 1.19 (ddd, J=2.4, 4.0, 6.6 Hz, 12H).³¹P NMR (162 MHz, CD₃CN) δ=149.77 (s), 149.63 (br s).

### Example 41

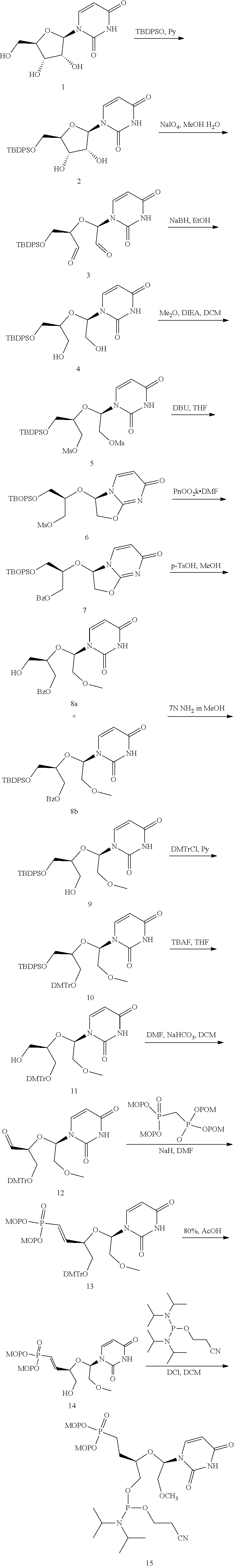

### Preparation of 2

Into a 5000-mL 3-necked round-bottom flask purged and maintained with an inert atmosphere of argon, was placed uridine (150.00 g, 614.24 mmol, 1.00 eq), pyridine (2.2 L), TBDPSCl (177.27 g, 644.95 mmol, 1.05 eq). The resulting solution was stirred overnight at room temperature. The resulting mixture was concentrated. The resulting solution was extracted with 3×1000 mL of dichloromethane and the organic layers combined. The resulting mixture was washed with 3×1 L of 0.5N HCl(aq.) and 2×500 mL of 0.5N NaHCO₃ (aq.). The resulting mixture was washed with 2×1 L of H₂O. The mixture was dried over anhydrous sodium sulfate. The solids were filtered out. The filtrate was concentrated. This resulted in 262 g (crude) 2. LC-MS (m/z) 483.00 [M+H]⁺; ¹H NMR (400 MHz, DMSO-d₆) δ 11.35 (d, J=2.2 Hz, 1H), 7.70 (d, J=8.1 Hz, 1H), 7.64 (m, 4H), 7.52-7.40 (m, 6H), 5.80 (d, J=4.1 Hz, 1H), 5.50 (d, J=5.1 Hz, 1H), 5.28 (dd, J=8.0, 2.2 Hz, 1H), 5.17 (d, J=5.3 Hz, 1H), 4.15-4.05 (m, 2H), 4.00-3.85 (m, 2H), 3.85-3.73 (m, 1H), 1.03 (s, 9H).

### Preparation of 3

Into a 10 L 3-necked round-bottom flask purged and maintained with an inert atmosphere of argon, was placed a solution of 2 (260.00 g, 538.7 mmol, 1.0 eq.) in MeOH (5000 mL). This was followed by the addition of a solution of NaIO₄ (126.8 g, 592.6 mmol, 1.1 eq.) in H₂O (1600 mL) in several batches at 0° C. The resulting solution was stirred for 1 hr at room temperature. The reaction was then quenched by the addition of 3 L of Na₂S203 (sat.) at 0° C. The resulting solution was extracted with 3×1 L of dichloromethane and the organic layers combined and dried over anhydrous sodium sulfate. The solids were filtered out. The filtrate was concentrated. This resulted in 290 g (crude) of 3 as a white solid.

### Preparation of 4

Into a 5 L 3-necked round-bottom flask purged and maintained with an inert atmosphere of argon, was placed 3 (290 g, 603.4 mmol, 1.0 eq), EtOH (3 L). This was followed by the addition of NaBH₄ (22.8 g, 603.4 mmol, 1.0 eq), in portions at 0° C. The resulting solution was stirred for 1 hr at room temperature. The reaction was then quenched by the addition of 2000 mL of water/ice. The resulting solution was extracted with 3×1000 mL of dichloromethane and the organic layers combined and dried over anhydrous sodium sulfate. The solids were filtered out. The filtrate was concentrated. This resulted in 230 g (crude) of 4 as a white solid. LC-MS: m/z 485.10 [M+H]⁺. ¹H NMR (400 MHz, DMSO-d₆) δ 11.28 (d, J=2.2 Hz, 1H), 7.63-7.37 (m, 11H), 5.84 (dd, J=6.4, 4.9 Hz, 1H), 5.44 (dd, J=8.0, 2.2 Hz, 1H), 5.11 (t, J=6.0 Hz, 1H), 4.78 (t, J=5.2 Hz, 1H), 3.65 (dd, J=11.4, 5.7 Hz, 1H), 3.60-3.52 (m, 5H), 3.18 (d, J=5.2 Hz, 1H), 0.96 (s, 9H).

### Preparation of 5

Into a 5000-mL 3-necked round-bottom flask purged and maintained with an inert atmosphere of argon, was placed a solution of 4 (120 g, 1 eq) in DCM (1200 mL). This was followed by the addition of DIEA (95.03 g, 3 eq) at 0 degrees C. To this was added methanesulfonic anhydride (129 g, 3 eq), in portions at 0° C. The resulting solution was stirred for 1 hr at room temperature. The reaction was then quenched by the addition of 1000 mL of water/ice. The resulting solution was extracted with 3×500 mL of dichloromethane and the organic layers combined and dried over anhydrous magnesium sulfate. The solids were filtered out. The filtrate was concentrated. This resulted in 160 g (crude) of 5 as a yellow solid.; LC-MS (m/z) 641.05[M+H]⁺.

### Preparation of 6

Into a 1 L round-bottom flask, was placed a solution of 5 (160.00 g, 1.00 equiv) in THF (1600 mL), DBU (108 g, 2.8 equiv). The resulting solution was stirred for 1 hr at 30° C. The reaction was then quenched by the addition of 3000 mL of water/ice. The resulting solution was extracted with 3×500 mL of dichloromethane and the organic layers combined and dried over anhydrous sodium sulfate. The solids were filtered out. The filtrate was concentrated. This resulted in 150 g (crude) of 6 as brown oil.; LC-MS:(ES, m/z): 567.25[M+H]+

¹HNMR (400 MHz, DMSO-d₆) δ 7.83 (d, J=7.4 Hz, 1H), 7.67-7.55 (m, 4H), 7.55-7.35 (m, 6H), 6.05 (dd, J=5.9, 1.7 Hz, 1H), 5.72 (d, J=7.4 Hz, 1H), 4.81 (dd, J=10.4, 5.8 Hz, 1H), 4.58-4.46 (m, 2H), 4.42 (p, J=5.2, 4.6 Hz, 1H), 4.33 (dd, J=10.6, 5.9 Hz, 1H), 3.79-3.70 (m, 2H), 3.23 (s, 3H), 0.98 (s, 9H).

### Preparation of 7

Into a 3000-mL round-bottom flask purged and maintained with an inert atmosphere of argon, was placed 6 (150.00 g, 201.950 mmol, 1. eq), DMF (1300.00 mL), potassium benzoate (44.00 g, 1.0 eq). The resulting solution was stirred for 1.5 hr at 80° C. The reaction was then quenched by the addition of 500 mL of water/ice. The resulting solution was extracted with 3×500 mL of dichloromethane The resulting mixture was washed with 3×1000 ml of H₂O. The resulting mixture was concentrated. The residue was applied onto a silica gel column with EA/PE (99:1). The collected fractions were combined and concentrated. This resulted in 40 g of 7 as yellow oil. LC-MS: m/z 571.20 [M+H]⁺; 1HNMR:(400 MHz, DMSO-d₆) δ 7.97-7.91 (m, 2H), 7.89 (d, J=7.4 Hz, 1H), 7.74-7.51 (m, 7H), 7.51-7.31 (m, 6H), 6.16 (m, 1H), 5.76 (d, J=7.4 Hz, 1H), 4.78 (m, 1H), 4.61 (m, 1H), 4.55-4.46 (m, 2H), 4.38 (m, 1H), 3.82 (d, J=5.0 Hz, 2H), 0.97 (s, 9H)

### Preparation of 8b

Into a 2-L round-bottom flask, was placed 7 (30.00 g, 1 eq), MeOH (1.20 L), p-toluenesulfonic acid (4.50 g, 0.5 eq). The resulting solution was stirred for 2 hr at 70° C. The reaction was then quenched by the addition of 3 L of NaHCO₃ (sat.). The pH value of the solution was adjusted to 7 with NaHCO₃ (sat.). The resulting solution was extracted with 3×1 L of ethyl acetate and the organic layers combined and dried over anhydrous sodium sulfate. The solids were filtered out. The filtrate was concentrated under vacuum. The crude product was purified by Flash-Prep-HPLC with the following conditions (IntelFlash-1): Column, silica gel; mobile phase, PE/EA=50/50 increasing to PE/EA=25/75 within 30; Detector, 254. This resulted in 11.5 g (3.1% yield in seven steps) 8b as a white solid. LC-MS: m/z 625.15[M+Na]⁺; ¹HNMR:(400 MHz, DMSO-d₆) δ 11.37 (d, J=2.3 Hz, 1H), 7.99-7.93 (m, 2H), 7.74-7.65 (m, 1H), 7.63-7.50 (m, 7H), 7.50-7.33 (m, 6H), 6.08 (t, J=6.0 Hz, 1H), 5.49 (m, 1H), 4.60 (m, 1H), 4.43 (m, 1H), 4.03-3.96 (m, 1H), 3.70 (d, J=5.3 Hz, 2H), 3.62-3.49 (m, 2H), 3.21 (s, 3H), 0.97 (s, 9H).

### Preparation of 9

Into a 2-L round-bottom flask, was placed 8b

(11.50 g). To the above 7M NH₃ (g) in MeOH (690.00 mL) was introduced in at 30° C. The resulting solution was stirred overnight at 30 degrees C. The resulting mixture was concentrated under vacuum. The crude product was purified by Flash with the following conditions (IntelFlash-1): Column, silica gel; mobile phase, PE/EA=60/40 increasing to PE/EA=1/99 within 60; Detector, 254. This resulted in 8.1 g (97% yield) of 9 as a white solid. LC-MS−: m/z 499.35 [M+H]⁺; ¹HNMR−: (300 MHz, DMSO-d₆) δ 11.31 (s, 1H), 7.64-7.50 (m, 5H), 7.48-7.35 (m, 6H), 6.02 (t, J=5.8 Hz, 1H), 5.45 (d, J=8.0 Hz, 1H), 4.80 (t, J=5.1 Hz, 1H), 3.58 (m, 7H), 3.27 (s, 3H), 0.96 (s, 9H).

### Preparation of 10

Into a 250-mL round-bottom flask, was placed 9 (8.10 g, 1 equiv), pyridine (80.0 mL), DMTr-Cl (7.10 g, 1.3eq). The flask was evacuated and flushed three times with Argon. The resulting solution was stirred for 2 hr at room temperature. The reaction was then quenched by the addition of 500 mL of NaHCO₃ (sat.). The resulting solution was extracted with 2×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 11.5 g (88% yield) of 10 as a white solid.; LC-MS: m/z 823.40 [M+Na]⁺; 1HNMR: (300 MHz, DMSO-d₆) δ 11.37 (s, 1H), 7.55-7.18 (m, 20H), 6.92-6.83 (m, 4H), 6.14 (t, J=5.9 Hz, 1H), 5.48 (d, J=8.0 Hz, 1H), 3.74 (m, 7H), 3.57 (m, 4H), 3.25 (m, 5H), 0.84 (s, 9H).

### Preparation of 11

Into a 1000-mL round-bottom flask, was placed 10 (11.5 g, 1.00 eq), THF (280.00 mL), TBAF (14.00 mL, 1.00 eq). The resulting solution was stirred for 3 hr at room temperature. The reaction was then quenched by the addition of 1 L of water. The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 7.8 g (98% yield) of 11 as a white solid. LC-MS: m/z 561.20 [M−H]⁻; ¹HNMR: (300 MHz, DMSO-d₆) δ 11.32 (s, 1H), 7.66 (d, J=8.1 Hz, 1H), 7.52-7.39 (m, 2H), 7.39-7.20 (m, 7H), 6.96-6.83 (m, 4H), 6.17 (t, J=5.9 Hz, 1H), 5.63 (d, J=8.0 Hz, 1H), 4.63 (t, J=5.6 Hz, 1H), 3.90-3.46 (m, 9H), 3.26 (s, 5H), 3.19-2.98 (m, 2H).

### Preparation of 12

Into a 3-L round-bottom flask, was placed 11 (7.80 g, 1.00 eq), DCM (300.00 mL), NaHCO₃ (3.50 g, 3 eq). This was followed by the addition of Dess-Martin (7.06 g, 1.2 equiv) with stirring at 0° C., and the resulting solution was stirred for 20 min at 0° C. The resulting solution was stirred for 5 hr at room temperature. The reaction mixture was cooled to 0 degree c. with a water/ice bath. The reaction was then quenched by the addition of 500 mL of NaHCO₃:Na₂S₂O₃=1:1. The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 5.8 g (75% yield) of 12 as a white solid. LC-MS: m/z 558.80 [M−H]⁻; ¹HNMR−:(300 MHz, DMSO-d₆) δ 11.35-11.22 (m, 1H), 9.43 (s, 1H), 7.75 (d, J=8.1 Hz, 1H), 7.49-7.19 (m, 8H), 6.90 (m, 5H), 6.00 (t, J=5.9 Hz, 1H), 5.66 (m, 1H), 4.40 (m, 1H), 3.75 (s, 7H), 3.70-3.56 (m, 3H), 3.29 (d, J=3.7 Hz, 3H).

### Preparation of 13

Into a 250-mL 3-round-bottom flask, was placed THF (150.00 mL), NaH (1.07 g, 60% w, 3.00 equiv). The flask was evacuated and flushed three times with Argon, and the reaction mixture was cooled to −78° C. This was followed by the addition of [[(bis[[(2,2-dimethylpropanoyl)oxy]methoxy]phosphoryl)methyl([(2,2-dimethylpropanoyl)oxy]methoxy)phosphoryl]oxy]methyl 2,2-dimethylpropanoate (14.60 g, 2.6 eq, in 60 m L THF) dropwise with stirring at −78° C. in 10 min, and the resulting solution was stirred for 30 min at −78° C. This was followed by the addition of 12 (5.00 g, 1.00 eq, in 50 mL THF) dropwise with stirring at −78° C. in 10 min. The resulting solution was stirred for 4 hr at room temperature. The reaction was then quenched by the addition of 400 mL of NH₄Cl(sat.). The resulting solution was extracted with 3×400 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 7.2 g (crude) of 13 as a solid. LC-MS: m/z: 865.10 [M−H].⁻

### Preparation of 14

Into a 500-mL round-bottom flask, was placed 13

(6.00 g), H₂O (30.00 mL), AcOH (120.00 mL). The resulting solution was stirred for 1 hr at 50 degrees C. The reaction mixture was cooled to 0 degree c. with a water/ice bath. The reaction was then quenched by the addition of 2 L of NaHCO₃ (sat.). The pH value of the solution was adjusted to 7 with NaHCO₃ (sat.). The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 2.6 g (44% yield in two steps) of 14 as yellow oil. LC-MS: m/z 587.25 [M+Na]⁺; ¹HNMR:(300 MHz, DMSO-d₆) δ 11.31 (s, 1H), 7.73 (d, J=8.1 Hz, 1H), 6.63 (ddd, J=24.2, 17.2, 4.2 Hz, 1H), 6.14-5.96 (m, 2H), 5.65-5.48 (m, 5H), 5.09 (t, J=5.6 Hz, 1H), 4.17 (s, 1H), 3.65 (d, J=6.1 Hz, 2H), 3.52 (m, 2H), 3.27 (s, 3H), 1.15 (d, J=3.7 Hz, 18H); ³¹PNMR−:(162 MHz, DMSO-d₆) δ 17.96.

### Preparation of 15

Into a 250-mL 3-necked round-bottom flask, was placed DCM (60.00 mL), DCI (351.00 mg, 1.2 eq), 3-[[bis(diisopropylamino)phosphanyl]oxy]propanenitrile (971.00 mg, 1.3 eq), 4A MS. The flask was evacuated and flushed three times with Argon, and the reaction mixture was cooled to 0° C. This was followed by the addition of 14 (1.40 g, 1.00 eq, in 30 mL DCM) dropwise with stirring at 0° C. in 30 second. The resulting solution was stirred for 1 hr at room temperature. The reaction was then quenched by the addition of 50 mL of water. The resulting solution was extracted with 3×50 mL of ethyl acetate and the organic layers combined. The resulting mixture was washed with 3×50 ml of NaCl(sat.). The mixture was dried over anhydrous magnesium sulfate. The solids were filtered out. The filtrate was concentrated under vacuum. The crude product was purified by Prep-Archiral-SFC with the following conditions: Column: Ultimate Diol, 2*25 cm, 5 ₁¹Ìm; Mobile Phase A: CO₂, Mobile Phase B: ACN(0.2% TEA); Flow rate: 50 mL/min; Gradient: isocratic 30% B; Column Temperature (20° C.): 35; Back Pressure (bar): 100; Wave Length: 254 nm; RT1 (min): 2.58; Sample Solvent:MeOH--HPLC; Injection Volume: 1 mL; Number Of Runs: 4. This resulted in 1.31 g (65% yield) 15 as yellow oil. LC-MS: m/z 763.40 [M−H]⁻; 1HNMR−: (300 MHz, Acetonitrile-d₃) δ 9.05 (s, 1H), 7.51 (d, J=8.1 Hz, 1H), 6.64 (dddd, J=23.8, 17.1, 4.8, 1.9 Hz, 1H), 6.23-5.92 (m, 2H), 5.70-5.51 (m, 5H), 4.38 (d, J=4.9 Hz, 1H), 3.96-3.56 (m, 8H), 3.35 (s, 3H), 2.70 (m, 2H), 1.33-1.14 (m, 30H); 31 PNMR−:(Acetonitrile-d₃) δ 148.75, 148.53, 16.68.

### Example 42

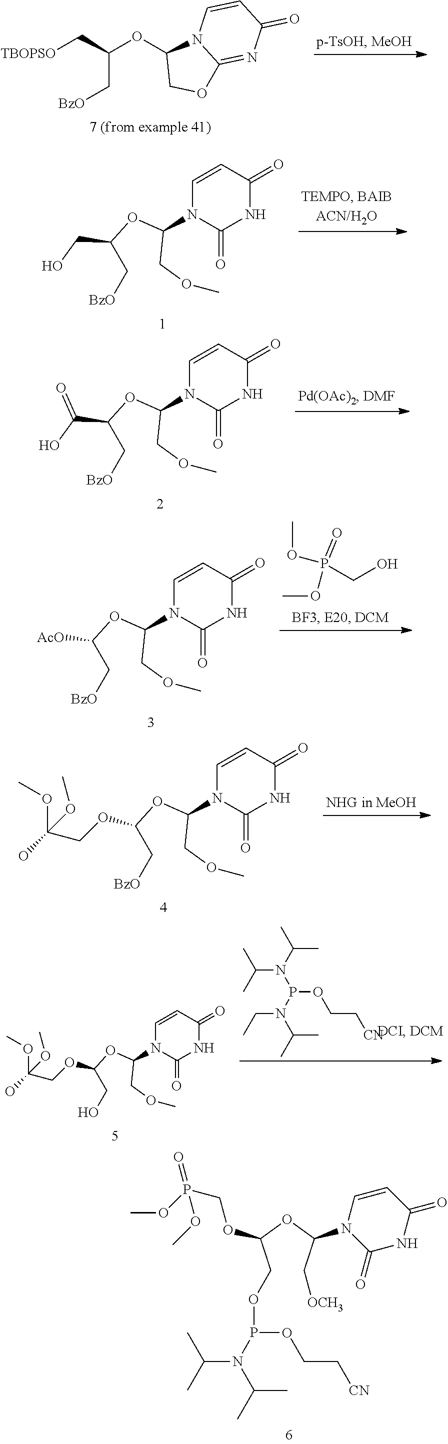

### Preparation of 1

A solution of 7 from Example 41 (23 g, 40.300 mmol, 1.00 equiv) and p-TsOH (9.02 g, 52.390 mmol, 1.3 equiv) in MeOH (1000 mL) was stirred for overnight at 40° C. under argon atmosphere. The reaction was quenched with sat. sodium bicarbonate (aq.) at 0 degrees C. The resulting mixture was extracted with EtOAc (2×500 mL). The combined organic layers were washed with water (2×500 mL), dried over anhydrous MgSO₄. After filtration, the filtrate was concentrated under reduced pressure. The residue was purified by reverse flash chromatography with the following conditions: column, C18 silica gel; mobile phase, ACN in water, 10% to 90% gradient in 30 min; detector, UV 254 nm. This resulted in 1 (5.3 g, 36.%) as a colorless oil.; LC-MS:(ES, m/z): 365 [M+H]⁺; ¹H-NMR: (300 MHz, DMSO-d₆) δ 11.20 (s, 1H), 8.09-7.78 (m, 2H), 7.63-7.50 (m, 2H), 7.51-7.35 (m, 2H), 5.95 (t, J=5.9 Hz, 1H), 5.51 (d, J=8.1 Hz, 1H), 4.73 (t, J=5.7 Hz, 1H), 4.41 (dd, J=11.9, 3.3 Hz, 1H), 4.17 (dd, J=11.9, 6.3 Hz, 1H), 3.69 (dq, J=10.1, 6.8, 6.3 Hz, 1H), 3.48-3.40 (m, 2H), 3.39-3.29 (m, 2H), 3.07 (s, 3H).

### Preparation of 2

Into a 250-mL 3-necked round-bottom flask, was placed 1 (7.00 g, 19.212 mmol, 1.00 equiv), ACN (60.00 mL), H₂O (60.00 mL), TEMPO (0.72 g, 4.611 mmol, 0.24 equiv), BAIB (13.61 g, 42.267 mmol, 2.20 equiv). The resulting solution was stirred for 1 overnight at 30° C. The reaction was then quenched by the addition of 200 mL of water/ice. The resulting solution was extracted with 2×200 mL of ethyl acetate, The resulting mixture was washed with 2×200 ml of water. The mixture was dried over anhydrous sodium sulfate and concentrated. The crude product was purified by Flash-Prep-HPLC with the following conditions (IntelFlash-1): Column, C18 silica gel; mobile phase, ACN/H₂O=5/95 increasing to ACN/H₂O=95/5 within 30 min; Detector, UV 254 nm; product was obtained. This resulted in 5 g (68.8%) of 2 as a solid. LC-MS:(ES, m/z): 379 [M+H]⁺; ¹H NMR (300 MHz, DMSO-d₆) δ 13.24 (s, 1H), 11.31 (d, J=2.2 Hz, 1H), 8.18-7.83 (m, 2H), 7.81-7.63 (m, 2H), 7.61-7.42 (m, 2H), 6.01 (t, J=6.0 Hz, 1H), 5.61 (dd, J=8.0, 2.2 Hz, 1H), 4.72-4.40 (m, 3H), 3.73-3.55 (m, 2H), 3.22 (s, 3H).

### Preparation of 3

Into a 250-mL round-bottom flask, was placed 2 (4.5 g, 11.894 mmol, 1.00 equiv), DMF (90.00 mL,), Pb(OAc)₄ (15.82 g, 35.679 mmol, 3.00 equiv). The resulting solution was stirred overnight at 30° C. The reaction was then quenched by the addition of 200 mL of water/ice. The resulting solution was extracted with 2×200 mL of ethyl acetate The resulting mixture was washed with 2×200 ml of water. The mixture was dried over anhydrous sodium sulfate and concentrated. The crude product was purified by Flash with the following conditions (IntelFlash-1): Column, C18 silica gel; mobile phase, ACN/H₂O=5/95 increasing to ACN/H₂O=95/5 within 30 min; Detector, UV 254 nm; product was obtained. This resulted in 4 g 3 as oil; LC-MS:(ES, m/z): 415 [M+Na]⁺; ¹H NMR (300 MHz, DMSO-d₆) δ 11.39 (s, 1H), 7.93 (dd, J=24.2, 7.6 Hz, 2H), 7.75-7.46 (m, 4H), 6.35-6.03 (m, 2H), 5.71-5.47 (m, 1H), 4.60-4.14 (m, 2H), 3.88-3.54 (m, 2H), 3.26 (d, J=6.7 Hz, 3H), 2.03 (d, J=49.7 Hz, 3H).

### Preparation of 4

Into a 250-mL 3-necked round-bottom flask purged and maintained with an inert atmosphere of argon, was placed 3 (4.00 g, 10.195 mmol, 1.00 eq), DCM (80.00 mL), dimethyl hydroxymethylphosphonate (22.85 g, 163.114 mmol, 16.00 eq), BF₃·Et₂O (28.94 g, 203.91 mmol, 20 eq). The resulting solution was stirred overnight at room temperature. The reaction was then quenched by the addition of 500 mL of water/ice. The resulting solution was extracted with 2×500 mL of ethyl acetate The resulting mixture was washed with 2×500 ml of water. The mixture was dried over anhydrous sodium sulfate and concentrated. The residue was applied onto a silica gel column with dichloromethane/methanol (20/1). This resulted in 2 g (41.5%) of 4 as a solid.

LC-MS:(ES, m/z): 490 [M+H₂O]+; 1H-NMR (300 MHz, DMSO-d₆) δ 11.39 (d, J=5.4 Hz, 1H), 7.96 (dt, J=11.5, 9.3 Hz, 2H), 7.81-7.40 (m, 4H), 6.29-5.98 (m, 1H), 5.56 (dd, J=12.2, 8.1 Hz, 1H), 5.28-4.99 (m, 1H), 4.29 (dp, J=25.1, 5.9 Hz, 2H), 4.16-3.84 (m, 2H), 3.75-3.53 (m, 7H), 3.28 (d, J=12.5 Hz, 2H).

### Preparation of 5

Into a 100-mL round-bottom flask, was placed 4 (2.00 g, 4.234 mmol, 1.00 equiv), 7M NH₃ (g) in THF (20.00 mL) was added. The resulting solution was stirred overnight at 25° C. The resulting mixture was concentrated under vacuum. The crude product was purified by prep-sfc Column: Lux Sum i-Cellulose-5, 3*25 cm, 5 m; Mobile Phase A: CO₂, Mobile Phase B:MeOH(0.1% 2M NH₃-MEOH); Flow rate: 70 mL/min; Gradient: isocratic 50% B; Column Temperature (25° C.): 35; Back Pressure (bar): 100; Wave Length: 220 nm; RT1 (min): 3.75; RT2 (min): 4.92; Sample Solvent:MeOH:DCM=1:1; Injection Volume: 1 mL; Number Of Runs: 15, This resulted in 330 mg (21.2%) of 5 as a solid. 1H-NMR−: (300 MHz, DMSO-d₆) δ 11.14 (s, 1H), 7.63 (d, J=8.1 Hz, 1H), 6.06 (t, J=5.9 Hz, 1H), 5.64 (d, J=8.0 Hz, 1H), 4.89 (s, 1H), 4.63 (t, J=5.3 Hz, 1H), 3.98 (d, J=9.8 Hz, 2H), 3.70 (dd, J=10.7, 1.2 Hz, 8H), 3.63 (dd, J=6.0, 3.2 Hz, 1H), 3.29 (s, 3H).

### Preparation of 6

To a stirred solution of 3-{[bis(diisopropylamino)phosphanyl]oxy}propanenitrile (324.10 mg, 1.075 mmol, 1.2 equiv) and 1H-imidazole-4,5-dicarbonitrile (126.99 mg, 1.075 mmol, 1.2 equiv) in DCM (10 mL) was added 5 (330 mg, 0.9 mmol, 1.00 eq) dropwise at 25° C. under argon atmosphere. The resulting mixture was stirred for 30 min at 25 degrees C. The reaction was quenched with water/ice. The resulting mixture was extracted with EtOAc (2×10 mL). The combined organic layers were washed with water (2×10 mL), dried over anhydrous MgSO₄. After filtration, the filtrate was concentrated under reduced pressure. Column: Ultimate Diol, 2*25 cm, 5 m; Mobile Phase A: CO₂, Mobile Phase B: ACN; Flow rate: 50 mL/min; Gradient: isocratic 30% B; Column Temperature (25° C.): 35; Back Pressure (bar): 100; Wave Length: 254 nm; RT1 (min): 3.95; Sample Solvent: ACN; Injection Volume: 1 mL; Number Of Runs: 10, This resulted in 6 (349 mg, 68.4%) as a light yellow oil. LC-MS:(ES, m/z): 567.25 [M+H]⁺; 1H-NMR: (300 MHz, DMSO-d₆) δ 11.38 (s, 1H), 7.64 (dd, J=8.0, 1.3 Hz, 1H), 6.09 (dt, J=5.8, 3.4 Hz, 1H), 5.65 (dd, J=8.0, 3.2 Hz, 1H), 4.83 (q, J=5.5 Hz, 1H), 4.03 (dt, J=9.7, 2.2 Hz, 2H), 3.83-3.40 (m, 14H), 3.30 (s, 3H), 2.77 (t, J=5.9 Hz, 2H), 1.12 (ddd, J=9.2, 6.7, 1.7 Hz, 12H); ³¹P NMR (DMSO-d₆) δ 148.0, 147.6, 23.1

### Example 43

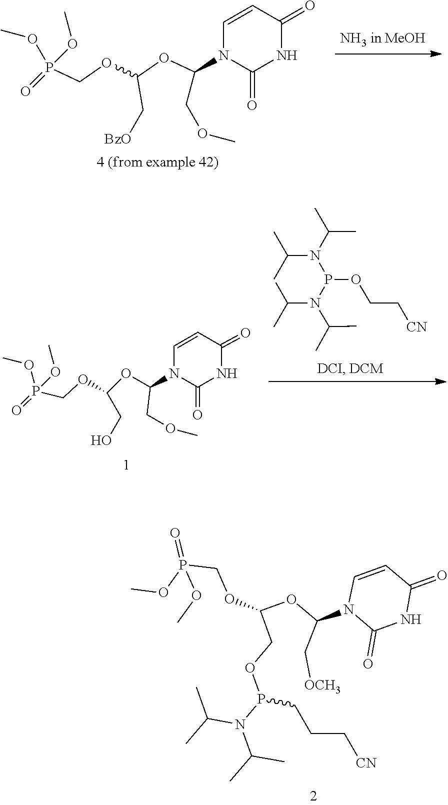

### Preparation of 1

Into a 100-mL round-bottom flask, was placed 2 4 from Example 42 (2.00 g, 4.234 mmol, 1.00 equiv), 7M NH₃ (g) in THF (20.00 mL) was added. The resulting solution was stirred overnight at 25° C. The resulting mixture was concentrated under vacuum. The crude product was purified by prep-sfc Column: Lux Sum i-Cellulose-5, 3*25 cm, 5 m; Mobile Phase A: CO₂, Mobile Phase B:MeOH (0.1% 2M NH₃-MeOH); Flow rate: 70 mL/min; Gradient: isocratic 50% B; Column Temperature (° C.): 35; Back Pressure (bar): 100; Wave Length: 220 nm; RT1 (min): 3.75; RT2 (min): 4.92; Sample Solvent:MeOH:DCM=1:1; Injection Volume: 1 mL; Number Of Runs: 15, This resulted in 320 mg (22.8%) of 1 as a solid. 1H-NMR− -14-3-40: (300 MHz, DMSO-d₆) δ 11.11 (s, 1H), 7.70 (d, J=8.0 Hz, 1H), 6.03 (t, J=6.1 Hz, 1H), 5.64 (d, J=8.0 Hz, 1H), 4.97 (s, 1H), 4.76 (t, J=5.3 Hz, 1H), 4.07-3.85 (m, 1H), 3.79 (dd, J=13.9, 9.3 Hz, 1H), 3.73-3.55 (m, 9H), 3.41 (d, J=5.0 Hz, 2H), 3.28 (s, 3H).

### Preparation of 2

To a stirred solution/mixture of 3-{[bis(diisopropylamino)phosphanyl]oxy}propanenitrile (517.58 mg, 1.717 mmol, 1.2 equiv) and 1H-imidazole-4,5-dicarbonitrile (202.79 mg, 1.717 mmol, 1.2 equiv) in DCM was added 1 (527 mg, 1.431 mmol, 1.00 eq.) dropwise at 25° C. under argon atmosphere. The resulting mixture was stirred for 30 min at 25° C. The reaction was quenched with Water/Ice. The resulting mixture was extracted with EtOAc (2×10 mL). The combined organic layers were washed with water (2×10 mL), dried over anhydrous MgSO₄. After filtration, the filtrate was concentrated under reduced pressure. Column: Ultimate Diol, 2*25 cm, 5 m; Mobile Phase A: CO2, Mobile Phase B: ACN(0.1% DEA)--HPLC--merk; Flow rate: 50 mL/min; Gradient: isocratic 30% B; Column Temperature (° C.): 35; Back Pressure (bar): 100; Wave Length: 254 nm; RT1 (min): 4.57; Sample Solvent: ACN; Injection Volume: 1 mL; Number Of Runs: 10 to afford 2 (264.8 mg, 31.7%) as a light yellow oil. LC-MS:(ES, m/z): 567.25 [M−H]⁻; 1H NMR (300 MHz, DMSO-d₆) δ 13.24 (s, 1H), 11.31 (d, J=2.2 Hz, 1H), 8.18-7.83 (m, 2H), 7.81-7.63 (m, 2H), 7.61-7.42 (m, 2H), 6.01 (t, J=6.0 Hz, 1H), 5.61 (dd, J=8.0, 2.2 Hz, 1H), 4.72-4.40 (m, 3H), 3.73-3.55 (m, 2H), 3.22 (s, 3H); ³¹P NMR (DMSO-d₆) δ 148.01, 147.67, 22.8.

### Example 44

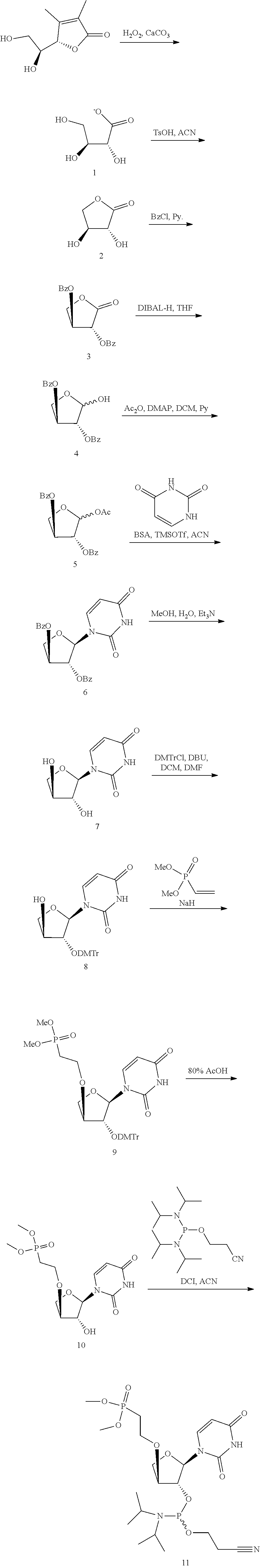

### Preparation of 1

To a stirred mixture of ascorbic acid (100.00 g, 567.78 mmol, 1.00 equiv) and CaCO₃ (113.0 g, 1129.02 mmol, 2 equiv) in H₂O (1.00 L) was added H₂O₂ (30%)(236.0 g, 6938.3 mmol, 12.22 equiv) dropwise at 0° C. The resulting mixture was stirred overnight at room temperature. The mixture was treat with charcoal and heat to 70 degrees until the no more peroxide was detected. The resulting mixture was filtered, the filter cake was washed with warm water (3×300 mL). The filtrate was concentrated under reduced pressure. The solid was diluted with MeOH (200 mL) and the mixture was stirred for 5 h. The resulting mixture was filtered, the filter cake was washed with MeOH (3×80 mL). The filtrate was concentrated under reduced pressure to afford L-threonate (86 g, 96.6%) as a white crude solid. 1H-NMR−: (300 MHz, Deuterium Oxide) δ 4.02 (dd, J=4.6, 2.4 Hz, 1H), 3.91 (ddt, J=7.6, 5.3, 2.2 Hz, 1H), 3.78-3.44 (m, 2H).

### Preparation of 2

Into a 5 L round-bottom flask were added L-threonate (70.00 g, 518.150 mmol, 1.00 equiv) and H₂O (2 L) at room temperature. The residue was acidified to pH=1 with Dowex 50wX8, H(+)-Form). The resulting mixture was stirred for 1 h at 70° C. The resulting mixture was filtered, the filter cake was washed with water (2×1 L). The filtrate was concentrated under reduced pressure. The solid was co-evaporated with (2×2 L). Then the solid was diluted with ACN (700.00 mL), and the TsOH(5.35 g, 31.089 mmol, 0.06 equiv) was added. The resulting mixture was stirred for 1 h at 80 degrees C. under air atmosphere. The resulting mixture was filtered, the filter cake was washed with ACN (2×500 mL). The filtrate was concentrated under reduced pressure to 2 (70 g, crude) as a yellow oil.

### Preparation of 3

To a stirred solution of (2 (70.0 g crude, 593.2 mmol, 1.00 eq.) in pyridine (280.00 mL) was added benzoyl chloride (207.62 g, 1.483 mol, 2.5 equiv) dropwise at 0° C. under argon atmosphere. The resulting mixture was stirred for 1 h at room temperature under argon atmosphere. The reaction was quenched by the addition of sat. NaHCO₃ (aq.) (500 mL) at 0 degrees C. The resulting mixture was extracted with CH₂Cl₂ (3×500 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc to afford (3 (80 g, 41.4%) as an off-white solid. LC-MS: (ES, m/z): 327 [M+H]+; 1H-NMR: (300 MHz, CDCl₃) δ 8.18-8.04 (m, 4H), 7.68-7.61 (m, 2H), 7.50 (tt, J=7.1, 1.4 Hz, 4H), 5.96-5.57 (m, 2H), 5.11-5.00 (m, 1H), 4.45-4.35 (m, 1H).

### Preparation of 4

To a stirred solution of 3 (125 g, 383.078 mmol, 1.00 eq) in THF(1.50 L) was added DIBAL-H (1M)(600 mL, 2 eq) dropwise at −78° C. under argon atmosphere. The resulting mixture was stirred for 1 h at −78 degrees C. under argon atmosphere. Desired product was detected by LCMS. The reaction was quenched with MeOH at 0° C. The resulting mixture was diluted with EtOAc (600 mL). Then the resulting mixture was filtered, the filter cake was washed with EtOAc (3×800 mL). The filtrate was concentrated under reduced pressure. This resulted in 4 (73 g, crude) as a colorless solid. LC-MS: (ES, m/z): 392 [M+Na+ACN]+; 1H-NMR−: (400 MHz, Chloroform-d) δ 8.22-7.99 (m, 8H), 7.62 (dtd, J=7.4, 4.4, 2.2 Hz, 4H), 7.48 (td, J=7.8, 2.4 Hz, 8H), 5.87 (d, J=4.3 Hz, 1H), 5.77 (dt, J=6.6, 3.6 Hz, 1H), 5.56 (d, J=4.9 Hz, 2H), 5.50 (t, J=4.3 Hz, 1H), 4.73 (s, 1H), 4.63 (ddd, J=10.4, 7.9, 6.1 Hz, 2H), 4.28 (dd, J=10.3, 3.8 Hz, 1H), 3.99 (dd, J=10.6, 3.2 Hz, 1H).

### Preparation of 5

To a stirred solution of (4 (73.00 g, 222.344 mmol, 1.00 equiv) and DMAP (271.63 mg, 2.223 mmol, 0.01 equiv) and pyridine(365.00 mL) in DCM(365.00 mL) were added Ac₂O(24.97 g, 244.6 mmol, 1.1 equiv) dropwise at 0 degrees C. under argon atmosphere. The resulting mixture was stirred for 1 h at room temperature under argon atmosphere. The reaction was quenched with sat. NaHCO₃ (aq.) at 0 degrees C. The resulting mixture was extracted with CH₂Cl₂ (3×500 mL). The combined organic layers were washed with sat. CuSO₄ (3×200 mL), dried over anhydrous Na₂SO₄. After filtration, the filtrate was concentrated under reduced pressure. The residue was purified by silica gel column chromatography, eluted with PE/EtOAc to afford 5 (60 g, 73%) as a colorless oil. LC-MS: (ES, m/z): 434 [M+Na+ACN]⁺; 1H-NMR: (400 MHz, Chloroform-d) δ 8.17-8.02 (m, 8H), 7.63 (tddd, J=7.9, 6.6, 3.2, 1.6 Hz, 4H), 7.57-7.44 (m, 8H), 6.66 (d, J=4.5 Hz, 1H), 6.40 (s, 1H), 5.83-5.53 (m, 4H), 4.67 (ddd, J=23.4, 10.5, 6.2 Hz, 2H), 4.24 (dd, J=10.5, 3.8 Hz, 1H), 4.19-4.01 (m, 1H), 2.18 (s, 3H), 2.06 (d, J=3.2 Hz, 3H).

### Preparation of 6

To a stirred mixture of 5 (50.00 g, 135.005 mmol, 1.00 eq) and uracil (15.13 g, 135.005 mmol, 1 eq) in can (500.00 mL) was added BSA (54.81 g, 270.010 mmol, 2 eq) in portions at room temperature under air atmosphere. The resulting mixture was stirred for 1 h at 60° C. under argon atmosphere. After that, the TMSOTf (90.02 g, 405.0 mmol, 3 eq) was added dropwise at 0° C. The resulting mixture was stirred for 2 h at 60° C. under argon atmosphere. The mixture was neutralized to pH=7 with saturated NaHCO₃ (aq.) at 0° C. The resulting mixture was extracted with CH₂Cl₂ (3×400 mL). The combined organic layers were washed with brine (2×400 mL), dried over anhydrous Na₂SO₄. After filtration, the filtrate was concentrated under reduced pressure. The residue was purified by silica gel column chromatography, eluted with PE/EtOAc (1:1) to afford 6 (43 g, 75.4%) as a white solid. LC-MS: (ES, m/z): [M+H]⁺; 423 464 [M+H+ACN]⁺; 1H-NMR−: (300 MHz, Chloroform-d) δ 9.08-8.89 (m, 1H), 8.17-7.94 (m, 4H), 7.70-7.43 (m, 7H), 6.19 (d, J=1.9 Hz, 1H), 5.84-5.71 (m, 2H), 5.62 (td, J=3.3, 2.8, 1.4 Hz, 1H), 4.59-4.44 (m, 2H), 4.14 (q, J=7.2 Hz, 1H).

### Preparation of 7

A solution of 6 (52.00 g, 123.108 mmol, 1 eq) was dissolved in 642 ml of MeOH/H₂O/TEA (5:1:1) at room temperature and heat to reflux until no more starting material was detected (2˜3 h). The resulting mixture was concentrated under reduced pressure. The residue was dissolved in EtOAc (600 mL) and the organic layer was extracted with water (5×800 mL). The aqueous layer was concentrated under vacuum to afford 7 (21 g, crude) as a off-white solid. The crude product was used in the next step directly without further purification. LC-MS−: (ES, m/z): 213 [M−H]⁻; 1 H-NMR: (300 MHz, DMSO-d₆) δ 11.26 (s, 1H), 7.68 (d, J=8.1 Hz, 1H), 5.75 (s, 1H), 5.65 (d, J=1.2 Hz, 1H), 5.59 (d, J=8.1 Hz, 1H), 5.39 (s, 1H), 4.10-3.97 (m, 4H).

### Preparation of 8

To a stirred mixture of 7 (16.00 g, 74.705 mmol, 1.00 equiv) and DBU (22.75 g, 149.409 mmol, 2 equiv) in DCM (80.00 mL) and DMF (200.00 mL) was added DMTr-Cl (7.88 g, 25.680 mmol, 1.1 equiv) dropwise at room temperature under argon atmosphere. The resulting mixture was stirred for 2 h at room temperature under argon atmosphere. The reaction was quenched by the addition of sat. NaHCO₃ (aq.) (100 mL) at 0 degrees C. The resulting mixture was extracted with EtOAc (3×60 mL). The combined organic layers were washed with brine (2×50 mL), dried over anhydrous Na₂SO₄. After filtration, the filtrate was concentrated under reduced pressure. The residue was purified by silica gel column chromatography, eluted with PE (0.5% TEA)/EtOAc (2:3) to afford 8 (25 g, 64.8%) as a off-white solid.; LC-MS: (ES, m/z): 515 [M−H]⁻; ¹H-NMR: (400 MHz, DMSO-d₆) δ 11.33 (s, 1H), 7.57 (d, J=8.1 Hz, 1H), 7.45-7.13 (m, 9H), 6.86 (t, J=8.5 Hz, 4H), 5.94 (d, J=1.7 Hz, 1H), 5.58 (d, J=8.1 Hz, 1H), 5.15 (d, J=2.6 Hz, 1H), 3.97-3.79 (m, 3H), 3.73 (d, J=2.3 Hz, 6H), 3.33 (d, J=2.5 Hz, 1H).

### Preparation of 9

To a stirred solution of 8 (6.00 g, 11.616 mmol, 1.00 eq) in THF (240.00 mL) was added NaH (60%) (1.40 g, 35.003 mmol, 3 eq) dropwise at 0° C. under argon atmosphere. The resulting mixture was stirred for 30 min at 0 degrees C. under argon atmosphere. Then the dimethyl ethenylphosphonate (15.81 g, 116.2 mmol, 10.00 eq) was added and the resulting mixture was stirred overnight at room temperature under argon atmosphere. The reaction was quenched with sat. NH₄C1 (aq.) at room temperature. The resulting mixture was extracted with EtOAc (3×100 mL). The combined organic layers were washed with brine (3×80 mL), dried over anhydrous Na₂SO₄. After filtration, the filtrate was concentrated under reduced pressure. The residue was purified by reverse flash chromatography with the following conditions: column, C18 mobile phase, ACN in water, 5% to 95% gradient in 30 min; detector, UV 254 nm to afford 9 (3.65 g, 48.15%) as a white solid.

LC-MS: (ES, m/z): 675 [M+Na]+; 1H-NMR−: (300 MHz, DMSO-d₆) δ 11.39 (s, 1H), 7.44-7.36 (m, 3H), 7.34-7.21 (m, 7H), 6.93-6.83 (m, 4H), 6.08 (d, J=2.0 Hz, 1H), 5.55 (d, J=8.1 Hz, 1H), 4.08 (d, J=11.0 Hz, 1H), 3.92 (d, J=2.0 Hz, 1H), 3.82-3.71 (m, 7H), 3.57 (dd, J=10.9, 3.6 Hz, 6H), 3.30-3.23 (m, 1H), 3.06-2.86 (m, 2H), 1.96 (dt, J=18.1, 7.1 Hz, 2H).

### Preparation of 10

A solution of 9 (2.80 g, 4.3 mmol, 1.00 equiv) in AcOH (12.00 mL) and H₂O (3.00 mL) was stirred for overnight at room temperature under air atmosphere. The reaction was quenched with sat. NaHCO₃ (aq.) at 0 degrees C. The resulting mixture was washed with 3×20 mL of CH₂Cl₂. The product in the water layer. The water layer was concentrated under reduced pressure. The product was purified by Prep-SFC with the following conditions (Prep SFC80-2): Column, Green Sep Basic, 3*15 cm; mobile phase, CO₂ (70%) and IPA (0.5% 2M NH₃-MeOH)(30%); Detector, UV 254 nm; product was obtained. This resulted in 870 mg (57.89%) of 10 as a white solid. LC-MS: (ES, m/z): 351 [M+Na]+; 1H-NMR−: (300 MHz, DMSO-d₆) δ 11.28 (s, 1H), 7.56 (d, J=8.1 Hz, 1H), 5.86 (d, J=4.4 Hz, 1H), 5.65 (d, J=1.6 Hz, 1H), 5.56 (d, J=8.1 Hz, 1H), 4.17 (d, J=10.1 Hz, 1H), 4.10 (d, J=4.3 Hz, 1H), 4.00 (dd, J=10.1, 3.9 Hz, 1H), 3.87 (dt, J=4.1, 1.3 Hz, 1H), 3.72-3.49 (m, 8H), 2.08 (dd, J=7.1, 2.8 Hz, 1H), 2.05-1.96 (m, 1H).

### Preparation of 11

Into a 250 mL 3-necked round-bottom flask were added Molecularsieve and ACN (30.00 mL) at room temperature. The resulting mixture was stirred for 10 min at room temperature under argon atmosphere. Then to the stirred solution were added 3-[[bis(diisopropylamino)phosphanyl]oxy]propanenitrile (1058.46 mg, 3.512 mmol, 1.5 equiv) and DCI (359.12 mg, 3.043 mmol, 1.30 equiv). Then the dimethyl 10 (820.00 mg, 2.341 mmol, 1.00 equiv) in 30 mL ACN was added dropwise at room temperature under argon atmosphere. The resulting mixture was stirred for 1 h at room temperature under argon atmosphere. The resulting mixture was diluted with CH2Cl2 (60 mL). The combined organic layers were washed with water (3×40 mL) after filtration, dried over anhydrous MgSO4. After filtration, the filtrate was concentrated un der reduced pressure. The residue was purified by Prep-TLC (0.5% TEA in PE/10% EtOH in EtOAc 1:9) to afford 11 (800 mg, 62.1%) as a colorless oil. LC-MS: (ES, m/z): 549 [M−H]⁻; 1H-NMR: (300 MHz, DMSO-d₆) δ 11.34 (s, 1H), 7.61 (dd, J=8.1, 1.7 Hz, 1H), 5.80 (dd, J=15.0, 1.8 Hz, 1H), 5.60 (d, J=8.1 Hz, 1H), 4.48-4.23 (m, 2H), 4.17-3.98 (m, 2H), 3.88-3.73 (m, 2H), 3.72-3.51 (m, 10H), 2.79 (q, J=5.9 Hz, 2H), 2.07 (dtt, J=17.9, 7.1, 3.2 Hz, 2H), 1.15 (ddd, J=6.3, 3.8, 2.1 Hz, 12H); ³¹P NMR (DMSO-d₆) δ 149.71, 149.35, 30.85, 30.75

### Example 45

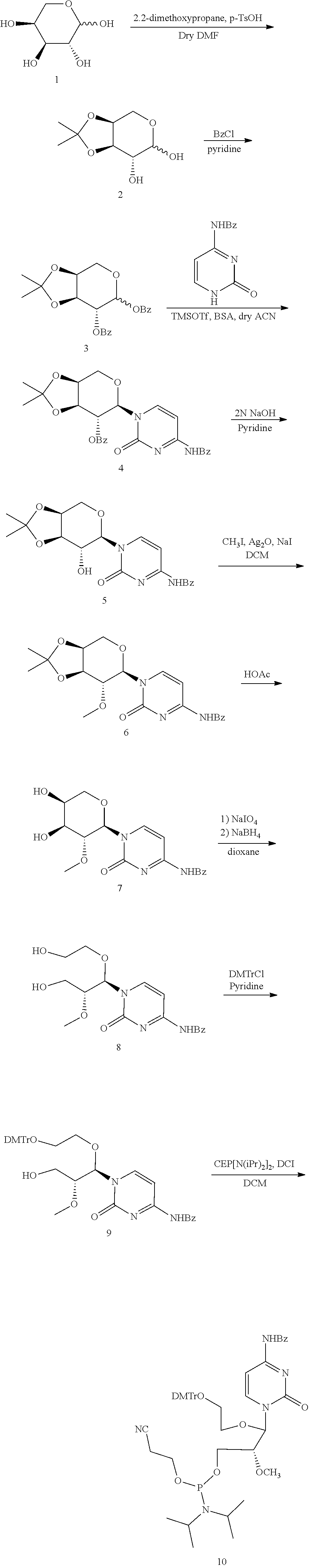

Preparation of 2: (J. Chem. Soc., Perkin Trans. 1, 1992, 1943-1952) To a solution of 1 (150.0 g, 1.0 mol) in DMF (2.0 L) was added 2,2-dimethoxypropane (312.0 g, 3.0 mol) and p-TsOH (1.7 g, 10.0 mmol), then the reaction mixture was stirred at r.t. for 4 h, after the reaction, the solvent was concentrated to give the crude products which was used directly to next step.

Preparation of 3: (J. Chem. Soc., Perkin Trans. 1, 1992, 1943-1952) To a solution of 2 (190.0 g, 1.0 mol) in pyridine (2.0 L) was added BzCl (560.0 g, 4.0 mol) then the reaction mixture was stirred at r.t. for 2 h, after the reaction, the reaction mixture was poured into the ice water, EA was added for extraction, and the organic phase was washed with brine, dried over Na₂SO₄ and concentrated to give the crude product which was purified by silica gel column (EA:PE=1:5 to 1:1) to give 3 (350.0 g, 87.9% yield), ESI-LCMS: m/z=421.2 [M+Na]⁺.

Preparation of 4: (J. Chem. Soc., Perkin Trans. 1, 1992, 1943-1952) to a solution of 3 (240.0 g, 815.5 mmol) in MeCN (3.0 L) was added N-(2-oxo-1H-pyrimidin-4-yl) benzamide (193.0 g, 897.0 mmol) and BSA (496.6 g, 2.4 mol). then the reaction mixture was stirred at 50° C. for 30 min, then the reaction mixture was cooled to 0° C., and the TMSOTf (271.5 g, 1.2 mol) was added into the mixture at 0° C., then the reaction mixture was stirred at 70° C. for 2 h, after the reaction, the solvent was concentrated to give an oil, then the oil was poured into the solution of NaHCO₃ maintaining the mixture was slightly alkaline, EA was added for extraction, and the organic phase was washed with brine, dried over Na₂SO₄ and concentrated to give the crude product which was purified by silica gel column (EA:PE=1:3 to 1:1) to give 4 (180.0 g, 44.9% yield). ESI-LCMS: m/z=491.2 [M+H]⁺; ¹H NMR (400 MHz, DMSO-d₆) δ 11.19 (s, 1H), 8.20 (d, J=7.6 Hz, 1H), 8.01-7.84 (m, 4H), 7.73-7.57 (m, 2H), 7.50 (dt, J=10.4, 7.7 Hz, 4H), 7.40 (d, J=7.4 Hz, 1H), 6.03 (d, J=9.4 Hz, 1H), 5.33 (dd, J=9.4, 7.3 Hz, 1H), 4.66 (dd, J=7.3, 5.3 Hz, 1H), 4.45-4.35 (m, 2H), 4.22 (dd, J=13.7, 2.5 Hz, 1H), 1.58 (s, 3H), 1.34 (s, 3H).

Preparation of 5: To a solution of 4 (78.0 g, 158.7 mmol) in pyridine (800.0 mL) was added a solution of NaOH (6.3 g, 158.7 mmol) in a mixture solvent of H₂O and MeOH (4:1, 2N), Then the reaction mixture was stirred at 0° C. for 20 min, LC-MS and TLC show that the raw material was disappeared, then the mixture was pour into a solution of NH₄Cl, EA was added for extraction, and the organic phase was washed with brine, dried over Na₂SO₄ and concentrated to give the crude product, which was purified by silica gel column (DCM:MeOH=30:1 to 10:1) to give 5 (56.0 g, 91.0% yield). ESI-LCMS: m/z=388.1 [M+H]⁺; ¹H NMR (400 MHz, DMSO-d₆) δ 11.29 (s, 1H), 8.16 (d, J=7.6 Hz, 1H), 8.08-7.99 (m, 2H), 7.67-7.60 (m, 1H), 7.53 (t, J=7.6 Hz, 2H), 7.35 (d, J=7.6 Hz, 1H), 5.63 (d, J=6.1 Hz, 1H), 5.51 (d, J=9.5 Hz, 1H), 4.35-4.13 (m, 3H), 3.78 (dt, J=9.6, 6.5 Hz, 1H), 3.19 (d, J=5.1 Hz, 1H), 1.53 (s, 3H), 1.32 (s, 3H).

Preparation of 6: To a solution of 5 (15.0 g, 38.7 mmol) in DCM (200.0 mL) was added Ag₂O (35.8 g, 154.8 mmol), CH₃I (54.6 g, 387.2 mmol) and NaI (1.1 g, 7.7 mmol), then the reaction mixture was stirred at r.t. overnight, after the reaction, filtrate was obtained through filtration, and the filtrate concentrated the solvent to obtain the product 6 (13.0 g, 75.2% yield,). ESI-LCMS: m/z=402.30 [M+H]⁺; ¹H NMR (400 MHz, DMSO-d₆) δ 11.30 (s, 1H), 8.22 (s, 1H), 8.00 (d, J=7.6 Hz, 2H), 7.71-7.20 (m, 4H), 5.56 (d, J=9.3 Hz, 1H), 4.33 (t, J=6.1 Hz, 1H), 4.26 (dd, J=6.2, 2.1 Hz, 1H), 4.20 (d, J=13.5 Hz, 1H), 3.98 (dd, J=13.5, 2.5 Hz, 1H), 3.66 (dd, J=9.3, 6.6 Hz, 1H), 3.34 (s, 3H), 1.57 (s, 3H), 1.32 (s, 3H).

Preparation of 7: To a solution of 6 (12.0 g, 29.9 mmol) was added CH₃COOH (120.0 mL), then the mixture was stirred at r.t. for 2 h, LC-MS and TLC showed that the raw material was disappeared, then the solvent was concentrated to get the crude product 7 (10.0 g, 83.3% yield,). ESI-LCMS: m/z=362.1 [M+H]⁺.

Preparation of 8: To a solution of 7 (10.0 g, 24.9 mmol) in dioxane:H₂O=3:1 (120.0 mL) was added NaIO₄ (8.8 g, 41.5 mmol), then the reaction mixture was stirred at r.t. for 2 h, LC-MS and TLC showed that the raw material was disappeared, then the reaction mixture was cooled to 0° C., and NaBH₄ (2.4 g, 41.5 mmol) was added into the mixture and stirred at 0° C. for 0.5 h, LC-MS and TLC showed that the raw material was disappeared, then NH₄C1 was added into the mixture to adjust pH to be slightly alkaline, and concentrated to give the crude product, which was purified by silica gel column (PE:EA=5:1 to 1:1) to give 8 (8.0 g, 79.5% yield). ESI-LCMS: m/z=364.1 [M+H]⁺; ¹H NMR (400 MHz, DMSO-d₆) δ 11.26 (s, 1H), 8.14 (d, J=7.5 Hz, 1H), 8.07-7.94 (m, 2H), 7.67-7.59 (m, 1H), 7.52 (t, J=7.6 Hz, 2H), 7.37 (s, 1H), 5.91 (d, J=6.0 Hz, 1H), 4.77 (t, J=5.6 Hz, 1H), 4.70 (t, J=5.1 Hz, 1H), 3.70 (ddd, J=11.5, 5.0, 2.5 Hz, 1H), 3.57-3.39 (m, 6H), 3.31 (s, 3H).

Preparation of 9: To a solution of 8 (4.0 g, 11.0 mmol) in pyridine (50.0 mL) was added DMTrCl (5.5 g, 16.5 mmol), then the reaction mixture was stirred at r.t. for 2 h, LC-MS showed that the raw material was 20.0% and The ratio of product to by-product was 3.5:1. then the solvent was concentrated to get residue which was purified by silica gel column to give the purified products and by-products was 5 g in total, then the product was purified by SFC to get 9 (3.0 g, 40.9% yield,). ESI-LCMS: m/z=666.2 [M+H]⁺; ¹H NMR (400 MHz, DMSO-d₆) δ 11.33 (s, 1H), 8.20 (d, J=7.4 Hz, 1H), 8.04 (d, J=7.7 Hz, 2H), 7.64 (t, J=7.4 Hz, 1H), 7.53 (t, J=7.6 Hz, 2H), 7.40 (d, J=7.8 Hz, 3H), 7.36-7.18 (m, 7H), 6.89 (d, J=8.4 Hz, 4H), 5.96 (d, J=5.7 Hz, 1H), 4.79 (t, J=5.7 Hz, 1H), 3.73 (s, 6H), 3.66-3.46 (m, 4H), 3.37 (s, 3H), 3.16 (ddd, J=10.1, 7.1, 3.0 Hz, 1H), 3.04 (dt, J=10.9, 3.4 Hz, 1H), 2.08 (s, 1H).

Preparation of 10: To a solution of 9 (2.8 g, 4.2 mmol) in DCM (30.0 mL) was added CEP[N(iPr)₂]₂ (1.3 g, 4.2 mmol) and DCI (601.2 mg, 5.1 mmol). The mixture was stirred at r.t. for 1 h. LC-MS showed 9 was consumed completely. The solution was washed with a solution of NaHCO₃ twice and washed with brine and dried over Na₂SO₄. Then concentrated to give a residue which was purified by Flash-Prep-HPLC with the following conditions (IntelFlash-1): Column, Cis silica gel; mobile phase, CH₃CN/H₂O (0.5% NH₄HCO₃)=1/1 increasing to CH₃CN/H₂O (0.5% NH₄HCO₃)=1/0 within 20.0 min, the eluted product was collected at CH₃CN/H₂O (0.5% NH₄HCO₃)=90/10; Detector, UV 254 nm. This resulted in to give 10 (2.8 g, 76.8% yield,). ESI-LCMS: m/z=866.2 [M+H]⁺; ¹H NMR (400 MHz, DMSO-d₆) δ 11.34 (s, 1H), 8.22 (d, J=7.4 Hz, 1H), 8.09-7.98 (m, 2H), 7.64 (t, J=7.4 Hz, 1H), 7.53 (t, J=7.6 Hz, 2H), 7.45 (d, J=7.3 Hz, 1H), 7.39 (d, J=7.5 Hz, 2H), 7.31 (t, J=7.6 Hz, 2H), 7.24 (t, J=9.1 Hz, 5H), 6.89 (d, J=8.8 Hz, 4H), 5.96 (d, J=6.1 Hz, 1H), 4.02-3.86 (m, 1H), 3.84-3.63 (m, 11H), 3.56 (dtq, J=13.3, 6.6, 3.5, 3.1 Hz, 3H), 3.37 (s, 2H), 3.16 (ddd, J=10.0, 6.8, 3.3 Hz, 1H), 3.04 (ddd, J=10.7, 5.5, 3.0 Hz, 1H), 2.75 (td, J=5.9, 2.3 Hz, 2H), 1.18-1.07 (m, 12H); ³¹P NMR (DMSO-d₆) δ 148.02 (d, J=12.0 Hz).

### Example 46

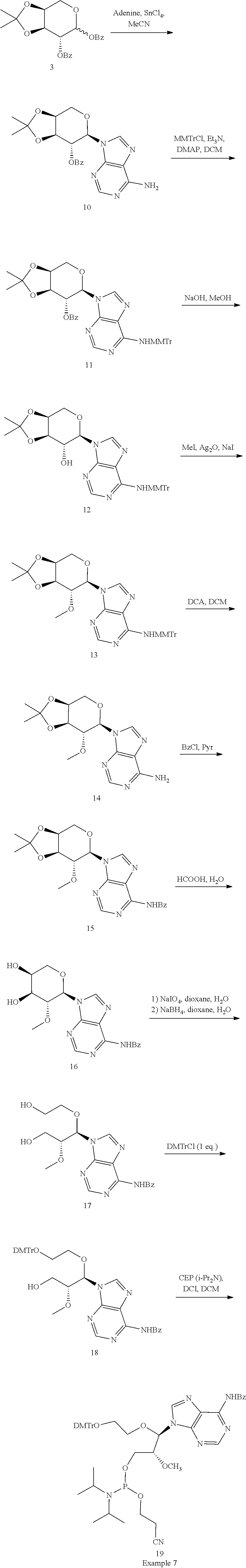

Preparation of 10: To the solution of 3 (200.0 g, 0.5 mol) in ACN (2000.0 mL) was added a solution of SnCl₄ in DCM (1000.0 mL) at 0° C. under N₂, and the reaction mixture was stirred at 0° C. for 4 h under N₂ atmosphere. Then the reaction solution was poured into saturated sodium bicarbonate solution, the resulting product was extracted with EA (3*500.0 mL). The combined organic layer was washed with water and brine, dried over Na₂SO₄, and concentrated to give the crude, which was purified by silica gel column (PE:EA=5:1 to 0:1) to give 10 (65.0 g, 31.4% yield) as a white solid. ESI-LCMS: m/z=412.0 [M+H]⁺; ¹H NMR (400 MHz, DMSO-d₆) δ 8.27 (s, 1H), 8.09 (s, 1H), 7.74-7.60 (m, 2H), 7.59-7.57 (m, 1H), 7.44-7.40 (m, 2H), 7.24 (s, 2H), 5.90 (d, J=9.6 Hz, 1H), 5.73 (dd, J=7.4 Hz, 1H), 4.63 (t, 1H), 4.50-4.30 (m, 2H), 4.21 (dd, J=13.6 Hz, 1H), 1.61 (s, 3H), 1.35 (s, 3H).

Preparation of 11: To a solution of 10 (40.0 g, 97.3 mmol) in DCM (500.0 mL) was added Et₃N (30.0 g, 297.0 mmol) and DMAP (1.2 g, 9.8 mmol) at r.t. The reaction mixture was replaced with N₂ over 3 times, then MMTrCl (45.0 g, 146.1 mmol) was added to the mixture. The reaction mixture was stirred at r.t. overnight. TLC and LC-MS showed that 10 was consumed, and the reaction mixture was added to an aqueous solution of NaHCO₃ in ice-water. Then extracted product with EA, washed the organic phase with brine, and dried the organic phase over Na₂SO₄, then concentrated to get 11 (66.5 g,) as a crude, used next step directly.

Preparation of 12: To a solution of 11 (66.5 g, 97.3 mmol) in pyridine (600.0 mL) was added 2N NaOH (H₂O:MeOH=4:1) (200.0 mL) at r.t. Then the reaction mixture was stirred at 0° C. for 30 min, LC-MS and TLC showed that the raw material was disappeared, then the mixture was poured into a solution of NH₄Cl, EA was added for extraction, and the organic phase was washed with brine, dried over Na₂SO₄ and concentrated to give the crude product which was purified by silica gel column (EA:PE=1:5 to 1:1) to give 12 (50.0 g, 88.7% yield for two step). ESI-LCMS: m/z=580.4 [M+H]⁺; ¹H NMR (400 MHz, DMSO-d₆) δ 8.44 (s, 1H), 7.92 (s, 1H), 7.36-7.16 (m, 13H), 6.89-6.80 (m, 2H), 5.59 (d, J=6.0 Hz, 1H), 5.35 (d, J=9.6 Hz, 1H), 4.32-4.12 (m, 4H), 4.08-3.95 (m, 3H), 3.72 (s, 3H), 1.99 (s, 3H), 1.54 (s, 3H), 1.32 (s, 3H), 1.17 (t, J=7.1 Hz, 3H).

Preparation of 13: To a solution of 12 (46.0 g, 79.4 mmol) in CH₃I (200.0 mL) was added Ag₂O (36.6 g, 158.4 mmol) and NaI (6.0 g, 42.5 mmol), then the reaction mixture was stirred at r.t. for 4 h, then the reaction mixture was filtrated and concentrated the solvent to obtain the product 13 (46.0 g, 97.6% yield), used next step directly. ESI-LCMS: m/z=594.3 [M+H]⁺.

Preparation of 14: To a stirred solution of DCA (22.5 mL) in DCM (750.0 mL) was added 13 (46.0 g, 77.5 mmol) and Et₃Si (185.0 mL) at r.t. And the reaction mixture was stirred at r.t. for 12 h. The reaction solution was evaporated to dryness under reduced pressure to give a residue, which was slurry with a solution of NaHCO₃ (50.0 mL) to get 14 (19.0 g, 76% yield), which was used next step directly.

Preparation of 15: To a solution of 14 (16.0 g, 49.7 mmol) in pyridine (200.0 mL) was added BzCl (9.0 g, 64.7 mmol) at 0° C. Then the reaction mixture was stirred at r.t. for 2 h. LC-MS showed 6 was consumed completely, then the mixture was cooled to 0° C., and a solution of NaOH in MeOH and H₂O (2N, 50.0 mL) was added into the reaction mixture, and the mixture was stirred for 1 h at 0° C., then the mixture was poured into a solution of NH₄Cl. The product was extracted with EA (300.0 mL) and the organic layer was washed with brine and dried over Na₂SO₄. Then the organic layer was concentrated to give a residue, which was purified by slurry with PE:EA (8:1, 900.0 mL) to get 15 (20.0 g, 95.0% yield). ESI-LCMS: m/z=426.2 [M+H]⁺; 1H NMR (400 MHz, DMSO-d₆) δ 11.21 (s, 1H), 8.77-8.69 (m, 2H), 8.06 (d, J=7.6 Hz, 2H), 7.65 (t, J=7.4 Hz, 1H), 7.56 (t, J=7.6 Hz, 2H), 7.34-7.23 (m, 4H), 7.23-7.12 (m, 5H), 6.89-6.80 (m, 4H), 5.90 (d, J=7.9 Hz, 1H), 4.36-4.29 (m, 1H), 4.06 (t, J=8.8 Hz, 1H), 3.92 (dd, J=25.0, 6.9 Hz, OH), 3.72 (d, J=1.0 Hz, 7H), 3.59 (dt, J=10.4, 6.6 Hz, 1H), 3.24 (s, 3H), 2.97 (d, J=7.7 Hz, 1H), 2.76 (q, J=5.5 Hz, 2H), 1.14 (dd, J=9.2, 5.7 Hz, 12H).

Preparation of 16: To a mixture solution of HCOOH (180.0 mL) and H₂O (20.0 mL) was added 15 (19.0 g, 44.7 mmol). The reaction mixture was stirred at r.t. for 4 h. LC-MS showed 15 was consumed completely. Then the reaction mixture was concentrated to give a residue which was purified by slurry with MeOH (100.0 mL) to get 16 (16.0 g, 92.7% yield) as a white solid. ESI-LCMS: m/z=385.9 [M+H]⁺; 1H NMR (400 MHz, DMSO-d₆) δ 11.21 (s, 1H), 8.77 (d, J=1.2 Hz, 2H), 8.09-8.02 (m, 2H), 7.70-7.61 (m, 1H), 7.56 (t, J=7.6 Hz, 2H), 5.56 (d, J=9.2 Hz, 1H), 5.21 (d, J=6.1 Hz, 1H), 4.94 (d, J=4.5 Hz, 1H), 4.18 (t, J=9.1 Hz, 1H), 4.09 (q, J=5.2 Hz, 1H), 3.88-3.71 (m, 4H), 3.21-3.14 (m, 6H).

Preparation of 17: To a solution of 16 (16.0 g, 41.4 mmol) in dioxane (200.0 mL) was added H₂O (32.0 mL), and NaIO₄ (9.7 g, 45.5 mmol), then the reaction mixture was stirred at r.t. for 1 h, LC-MS and TLC showed that the raw material was disappeared, then the reaction mixture was cooled to 0° C., and NaBH₄ (1.7 g, 45.5 mmol) was added into the mixture and stirred at 0° C. for 0.5 h, LC-MS and TLC showed that the intermediate state was disappeared, then the NH₄C1 was added into the mixture to adjust pH to be slightly alkaline, and concentrated at r.t. to give the crude product which was purified by silica gel column (DCM:MeOH=20:1 to 8:1) to give 17 (16.0 g, 99.5% yield). ESI-LCMS: m/z=388.0 [M+H]⁺; ¹H NMR (400 MHz, DMSO-d₆) δ 11.18 (s, 1H), 8.75 (s, 1H), 8.67 (s, 1H), 8.09-7.99 (m, 2H), 7.65 (t, J=7.4 Hz, 1H), 7.56 (t, J=7.6 Hz, 2H), 5.90 (d, J=7.6 Hz, 1H), 4.88 (t, J=5.7 Hz, 1H), 4.67 (t, J=5.5 Hz, 1H), 4.08-3.98 (m, 2H), 3.78 (ddd, J=12.1, 5.2, 3.1 Hz, 1H), 3.68-3.39 (m, 4H), 3.36 (s, OH), 3.20 (s, 3H), 1.99 (s, 1H), 1.17 (t, J=7.1 Hz, 1H).

Preparation of 18: To a solution of 17 (12.0 g, 31.0 mmol) in pyridine (50.0 mL) was added DMTrCl (11.5 g, 34.1 mmol), then the reaction mixture was stirred at r.t. for 2 h, LC-MS showed that the raw material was 15.0% remained and the ratio of product to by-product was 3.5:1. Then the reaction solution was poured into ice-water, and extracted with EA, wished with brine, dried over Na₂SO₄, filtered and concentrated to get residue which was purified by silica gel column to give the purified product and by-product were 13.0 g in total, then 4.0 g crude was purified by SFC to get 18 (3.3 g, 15.4% yield,). ESI-LCMS: m/z=690.3 [M+H]⁺; ¹H NMR (400 MHz, DMSO-d₆) δ 11.21 (s, 1H), 8.75 (s, 1H), 8.69 (s, 1H), 8.10-8.03 (m, 2H), 7.70-7.61 (m, 1H), 7.56 (t, J=7.6 Hz, 2H), 7.35-7.12 (m, 9H), 6.90-6.80 (m, 4H), 5.94 (d, J=7.5 Hz, 1H), 4.88 (t, J=5.6 Hz, 1H), 4.36 (t, J=5.1 Hz, 1H), 4.11 (dt, J=7.4, 3.6 Hz, 1H), 3.82 (ddd, J=11.9, 5.1, 3.1 Hz, 1H), 3.72 (d, J=1.3 Hz, 7H), 3.64 (ddd, J=11.9, 6.2, 4.2 Hz, 1H), 3.45 (qd, J=7.0, 4.9 Hz, 2H), 3.24 (s, 3H), 3.09 (ddd, J=9.9, 6.4, 3.2 Hz, 1H), 2.97 (ddd, J=9.9, 5.7, 3.2 Hz, 1H), 1.23 (s, OH), 1.06 (t, J=7.0 Hz, 1H).

Preparation of 19: To a suspension of 18 (3.3 g, 4.8 mmol) in DCM (40.0 mL) was added DCI (0.5 g, 4.0 mmol) and CEP[N(iPr)₂]₂ (1.6 g, 5.3 mmol). The mixture was stirred at r.t. for 0.5 h. LC-MS showed 10 was consumed completely. The solution was washed with a solution of NaHCO₃ twice and washed with brine and dried over Na₂SO₄. Then concentrated to give a residue which was purified by Flash-Prep-HPLC with the following conditions (IntelFlash-1): Column, Cis silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 19 (3.0 g, 3.9 mmol, 81.2% yield) as a white solid. ESI-LCMS: m/z=765.3 [M+H]⁺; ¹H NMR (400 MHz, DMSO-d₆) δ 11.22 (s, 1H), 8.80-8.71 (m, 2H), 8.11-8.04 (m, 2H), 7.65 (t, J=7.3 Hz, 1H), 7.56 (t, J=7.5 Hz, 2H), 7.36-7.24 (m, 4H), 7.24-7.15 (m, 5H), 6.89-6.82 (m, 4H), 5.92 (d, J=7.7 Hz, 1H), 4.34 (dt, J=7.5, 3.5 Hz, 1H), 4.08 (ddd, J=10.7, 7.3, 2.7 Hz, 1H), 4.03-3.89 (m, 1H), 3.80-3.72 (m, 1OH), 3.67-3.53 (m, 2H), 3.47 (dp, J=10.5, 3.4 Hz, 1H), 3.26 (s, 3H) 3.11 (ddd, J=10.3, 6.2, 3.5 Hz, 1H), 3.00 (q, J=6.6, 5.2 Hz, 1H), 2.77 (q, J=5.6 Hz, 2H), 2.08 (s, 1H), 1.15 (t, J=7.0 Hz, 12H).; ³¹P NMR (162 MHz, DMSO-d₆) δ 148.30, 147.99.

### Example 47

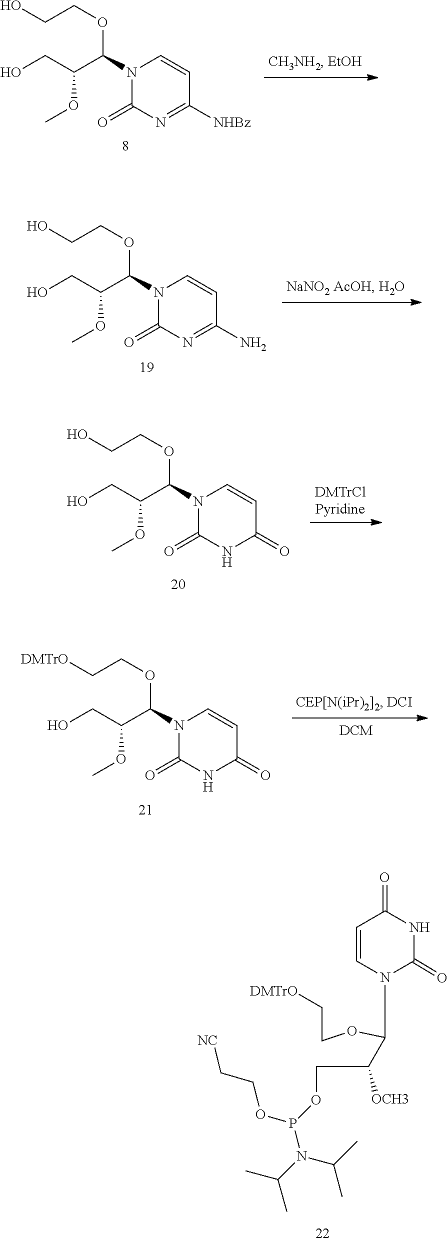

Preparation of 19: To a solution of 8 (8.0 g, 22.0 mmol) in EtOH (50.0 mL) was added a solution of CH₃NH₂ (50.0 mL), then the reaction mixture was stirred at r.t. for 4 h, after the reaction, the solvent was concentrated to give the crude, which was added into a mixture solvent of EA (20.0 mL) and PE (10.0 mL), then the mixture was stirred for 30 min and filtered to get 19 (5.5 g, 96.500 yield), which was used directly to next step.

Preparation of 20: (J. Chem. Soc., Perkin Trans. 1, 1992, 1943-1952) To a solution of 19 (5.0 g, 19.3 mmol) in H₂O (50.0 mL) and AcOH (50.0 mL) was added NaNO₂ (65.0 g, 772.0 mmol), then the reaction mixture was stirred at r.t. for 2 h, after the reaction, the reaction mixture was concentrated to give the crude product which was purified by silica gel column (DCM:MeOH=20:1 to 6:1) and MPLC (ACN: H₂O=0:100 to 10:90) to give 20 (3.0 g, 59.6% yield). ESI-LCMS: m/z=261.2 (M+H)⁺; ¹H NMR (400 MHz, DMSO-d₆) δ 11.29 (s, 1H), 7.66 (d, J=8.0 Hz, 1H), 5.67 (dd, J=17.5, 7.6 Hz, 2H), 4.74 (d, J=36.0 Hz, 2H), 3.86-3.63 (m, 1H), 3.58-3.40 (in, 6H).

Preparation of 21: To a solution of 20 (3.0 g, 11.5 mmol) in pyridine (30.0 mL) was added DMTrCl (3.9 g, 11.5 mmol), then the reaction mixture was stirred at r.t. for 2 h, LC-MS showed that the raw material was 20.00% and The ratio of product to by-product was 3:1, then the mixture was poured into a solution of NaHCO₃ (100.0 mL), and extracted with EA (100.0 mL), washed with brine and dried over Na₂SO₄, filtered and concentrated to get residue, which was purified by silica gel column to give The purified products and by-products were 5.0 g in total, then the product was purified by SFC to give 21 (1.8 g,). ESI-LCMS: m/z=561.2 [M+H]⁺; ¹H NMR (400 MHz, DMSO-d₆) δ 11.31 (s, 1H), 7.69 (d, J=8.1 Hz, 1H), 7.45-7.15 (m, 8H), 6.88 (d, J=8.5 Hz, 4H), 5.71 (d, J=6.8 Hz, 1H), 5.64 (d, J=8.0 Hz, 1H), 4.79 (t, J=5.5 Hz, 1H), 3.74 (s, 6H), 3.60 (s, 1H), 3.51 (d, J=5.5 Hz, 3H), 3.11 (d, J=6.7 Hz, 1H), 3.02 (d, J=7.0 Hz, 1H).

Preparation of 22: To a solution of 21 (1.8 g, 3.2 mmol) in DCM (20.0 mL) was added CEP[N(iPr)₂]₂ (1.0 g, 3.4 mmol) and DCI (321.0 mg, 2.7 mmol). The mixture was stirred at r.t. for 1 h. LC-MS showed 21 was consumed completely. The solution was washed with solution of NaHCO₃ twice and washed with brine and dried over Na₂SO₄. Then concentrated to give a residue, which was purified by Flash-Prep-HPLC with the following conditions (IntelFlash-1): Column, Cis silica gel; mobile phase, CH₃CN/H₂O (0.5% NH₄HCO₃)=1/1 increasing to CH₃CN/H₂O (0.5% NH₄HCO₃)=1/0 within 20.0 min, the eluted product was collected at CH₃CN/H₂O (0.5% NH₄HCO₃)=90/10; Detector, UV 254 nm. This resulted in to give 22 (2.0 g, 82% yield). ESI-LCMS: m/z=761.2 [M+H]⁺; ¹H NMR (400 MHz, DMSO-d₆) δ 11.35 (s, 1H), 7.73 (dd, J=8.0, 2.0 Hz, 1H), 7.39 (d, J=7.4 Hz, 2H), 7.35-7.18 (m, 7H), 6.94-6.82 (m, 4H), 5.81-5.74 (m, 1H), 5.67 (d, J=8.0 Hz, 1H), 4.11-3.85 (m, 1H), 3.82-3.67 (m, 11H), 3.67-3.50 (m, 5H), 3.17-3.09 (m, 1H), 3.09-3.01 (m, 1H), 2.74 (td, J=5.8, 2.9 Hz, 2H), 1.13 (dd, J=9.2, 6.7 Hz, 13H); ³¹P NMR (DMSO-d₆) δ 148.09 (d, J=41.8 Hz).

### Example 48

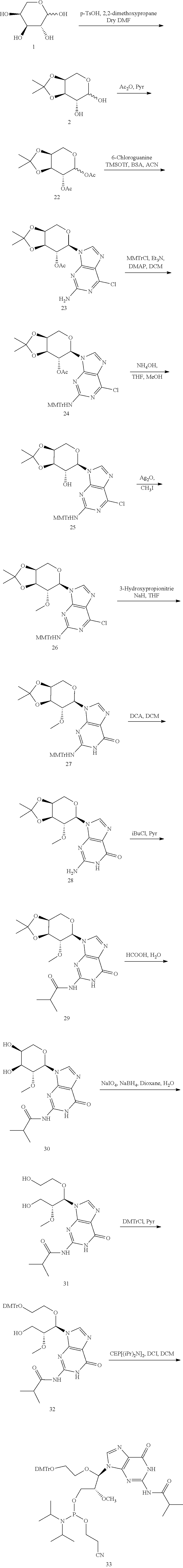

Preparation of 2 (J. Chem. Soc., Perkin Trans. 1, 1992, 1943-1952): To a solution of 1 (150.0 g, 999.1 mmol) in DMF (1000.0 mL) was added P-TsOH (1.7 g, 10.0 mmol), then 2,2-dimethoxy-propane (312.2 g, 3.0 mol) was added to the reaction mixture. The reaction mixture was stirred for 5 h at r.t. 90.0% 1 was consumed by TLC. Then NaHCO₃ (8.4 g, 99.9 mmol) was added to the reaction mixture, filtered out the solid after 30 min, and concentrated the organic phase by vacuum to obtain crude, which was purified by c.c. (PE:EA=1:1 to 0:1) to get compound 2 (115.0 g, 60.5% yield) as a white solid.

Preparation of 22: A solution of 2 (115.0 g, 604.6 mmol) in pyridine (600.0 mL) was cooled to 0° C., then Ac₂O (185.2 g, 1.81 mol) was added drop wise to the reaction mixture. The reaction was stirred for 2 h at r.t., and the raw material was consumed by TLC. The reaction solution was added into water, extracted product with EA. The organic phase was washed with brine, and dried the organic phase with Na₂SO₄, and concentrated to get 22 (150.0 g, 90.4% yield), which was used for next step directly. ¹H NMR (400 MHz, Chloroform-d) δ 6.20 (d, J=3.4 Hz, 1H), 5.66 (d, J=6.8 Hz, 1H), 5.17 (t, J=6.9 Hz, 1H), 5.10 (dd, J=7.0, 3.4 Hz, 1H), 4.40-4.25 (m, 3H), 4.21 (dd, J=7.0, 6.1 Hz, 1H), 4.16-4.02 (m, 3H), 3.95 (dd, J=12.9, 4.4 Hz, 1H), 2.17 (s, 1H), 2.15-2.03 (m, 12H), 1.56 (d, J=4.0 Hz, 6H), 1.37 (d, J=3.1 Hz, 6H).

Preparation of 23: To a solution of 22 (150.0 g, 546.9 mmol) in ACN (2200.0 mL) was added 6-chloroguanine (139.1 g, 820.4 mmol) and BSA (333.7 g, 1.6 mol) at r.t., then the reaction mixture was replaced with N₂ over 3 times. The reaction was stirred for 30 min at 50° C. After that, the reaction mixture was cooled to 0° C. under N₂. Then TMSOTf (182.1 g, 820.4 mmol) was added into the mixture. After addition, the reaction was stirred for 1.5 h at 70° C. TLC and LC-MS showed the raw material was consumed. Concentrated the most organic solvent by vacuum, then the residual was added to an aqueous solution of NaHCO₃ in ice-water, extracted product with EA (4.0 L), dried the organic phase over Na₂SO₄, and filtered and concentrated to get crude, which was purified by c.c. (DCM to DCM:EA=5:1) to get compound 23 (82.0 g, 35.0% yield,) as a white solid. ESI-LCMS: m/z=384.8 [M+H]⁺; ¹H NMR (400 MHz, DMSO-d₆) δ 8.23 (s, 1H), 7.04 (d, J=22.3 Hz, 2H), 5.57 (d, J=9.6 Hz, 1H), 5.40 (dd, J=9.6, 7.3 Hz, 1H), 4.48 (dd, J=7.4, 5.4 Hz, 1H), 4.40-4.30 (m, 2H), 4.11 (dd, J=13.6, 2.4 Hz, 1H), 1.81 (s, 3H), 1.55 (s, 3H), 1.34 (s, 3H).

Preparation of 24: To a solution of 23 (82.0 g, 192.3 mmol) in DCM (1000.0 mL) was added Et₃N (59.4 g, 576.9 mmol) and DMAP (2.4 g, 19.2 mmol) at r.t. The reaction mixture was replaced with N₂ over 3 times, then MMTrCl (90.9 g, 288.4 mmol) was added into the mixture. The reaction mixture was stirred at r.t. overnight. TLC and LC-MS showed that 92.0% raw material was consumed, and the reaction mixture was added to an aqueous solution of NaHCO₃ in ice-water, then extracted product with EA. Washed the organic phase with brine, and dried the organic phase over Na₂SO₄, then concentrated to get crude, which was purified by c.c. (DCM) to give compound 24 (110.0 g, 86.4% yield) as a white solid. ESI-LCMS: m/z=657.1 [M+H]⁺; ¹H NMR (400 MHz, DMSO-d₆) δ 8.21 (s, 1H), 7.37-7.31 (m, 4H), 7.29-7.23 (m, 6H), 7.20-7.15 (m, 2H), 6.86-6.80 (m, 2H), 5.75 (s, 1H), 5.23 (dd, J=9.6, 7.2 Hz, 1H), 4.85 (s, 1H), 4.44-4.16 (m, 3H), 3.71 (s, 4H), 1.70 (s, 3H), 1.49 (s, 3H), 1.31 (s, 3H).

Preparation of 25: To a solution of 24 (110.0 g, 164.3 mmol) in a mixed solvent of THF (500.0 mL) and MeOH (160.0 mL) was added NH₄OH (330.0 mL). The reaction mixture was stirred overnight at r.t., and the raw material was consumed by TLC and LC-MS. The reaction liquid was added into water, extracted product with EA. Washed the organic phase with brine, then dried the organic phase over Na₂SO₄, then concentrated to get the crude, which was purified by c.c. (PE:EA=10:1˜1:2) to give compound 25 (98.0 g, 94.2% yield) as a white solid. ESI-LCMS: m/z=615.1 [M+H]⁺; ¹H NMR (400 MHz, DMSO-d₆) δ 8.32 (s, 1H), 7.36 (dt, J=8.2, 1.4 Hz, 4H), 7.31-7.21 (m, 6H), 7.15 (t, J=7.2 Hz, 2H), 6.85-6.76 (m, 2H), 5.57 (d, J=4.6 Hz, 1H), 4.69 (s, 1H), 4.25 (dt, J=5.1, 2.4 Hz, 1H), 4.03 (q, J=7.1 Hz, 4H), 3.70 (s, 3H), 3.62-3.44 (m, 1H), 1.51 (s, 3H), 1.31 (s, 3H).

Preparation of 26 (Ref WO2011/95576, 2011, Al): To a solution of 25 (70.0 g, 114.0 mmol) in CH₃I (350.0 mL) was added Ag₂O (79.2 g, 342.0 mmol) at r.t. Then the reaction mixture was stirred for 4 h at r.t. TLC and LC-MS showed that the raw material was consumed. Filtered out the residue with diatomite, and concentrated the filtrate by vacuum to get crude, which was purified by c.c. (PE:EA=10:1˜1:1) to get compound 26 (28.0 g, 31.3% yield) as a white solid. ESI-LCMS: m/z=629.1 [M+H]⁺.

Preparation of 27: A solution of 3-hydroxy-propionitrile (15.6 g, 219.7 mmol) in THF (200.0 mL) was cooled to 0° C. The reaction mixture was replaced by N₂ over 3 times. Then NaH (12.4 g, 310.0 mmol, 60.0%) was added to the reaction mixture in turn. The reaction was stirred for 30 min at r.t., and then the reaction was cooled to 0° C. again. A solution of 26 (26.0 g, 33.0 mmol) in THF (150.0 mL) was added drop wise to the reaction mixture. Then the reaction mixture was stirred at r.t. overnight. TLC and LC-MS showed the raw material was consumed. The reaction liquid was added into water, extracted product with EA. The organic phase was washed with brine, and dried over Na₂SO₄, then concentrated to get the crude, which was purified by c.c. (DCM:MeOH=50:1˜30:1) to get compound 27 (18.0 g, 88.0% yield) as white solid. ESI-LCMS: m/z=610.7 [M+H]⁺; ¹H NMR (400 MHz, DMSO-d₆) δ 10.68 (s, 1H), 7.90 (s, 1H), 7.69 (s, 1H), 7.34-7.15 (m, 12H), 6.92-6.81 (m, 2H), 4.46 (d, J=9.5 Hz, 1H), 4.22 (dt, J=5.5, 2.5 Hz, 1H), 4.07 (t, J=6.4 Hz, 1H), 3.84 (dd, J=13.5, 2.1 Hz, 1H), 3.64-3.54 (m, 1H), 3.36 (dd, J=13.3, 2.8 Hz, 1H), 3.08 (s, 3H), 2.59 (t, J=6.0 Hz, 3H), 1.49 (s, 3H), 1.30 (s, 3H).

Preparation of 28 (Beigelman, Leonid; Deval, Jerome; Jin, Zhinan WO2014/209979, 2014, A1,): To a solution of 27 (18.0 g, 29.5 mmol) in DCM (300.0 mL) was added triethylsilane (70.0 mL) and DCA (10.0 mL) at r.t. Then the reaction mixture was stirred for 6 h at r.t., TLC and LC-MS showed that the raw material was consumed. Concentrated the almost organic solvent by vacuum, then PE (600.0 mL) was added to the reaction mixture. Filtered of the organic phase to get the solid, which was purified by MPLC (MeCN: H₂O=40:60 to 50:50) to get compound 28 (7.5 g, 75.0% yield) as a white solid. ESI-LCMS: m/z=338.3 [M+H]⁺; ¹H NMR (400 MHz, DMSO-d₆) δ 10.70 (s, 1H), 8.03 (s, 1H), 6.49 (s, 2H), 5.15 (d, J=9.6 Hz, 1H), 4.28 (d, J=5.1 Hz, 2H), 4.20 (d, J=13.6 Hz, 1H), 3.93 (ddd, J=13.3, 10.6, 3.7 Hz, 2H), 3.26 (s, 3H), 1.59 (s, 3H), 1.33 (s, 3H);

Preparation of 29: A solution of 28 (7.0 g, 20.6 mmol) in Pyr (150.0 mL) was cooled to 0° C. Then the reaction mixture was added i-BuCl (6.6 g, 61.8 mmol) drop wise. The reaction mixture was stirred for 30 min, TLC and LC-MS showed the raw material was consumed. The reaction liquid was added to ice-water, extracted product with EA. The organic phase was washed with brine, and dried over Na₂SO₄, and filtered and concentrated to get the crude, which was purified by c.c. (DCM:MeOH=100:1˜30:1) to get compound 29 (5.8 g, 68.6% yield) as a white solid. ESI-LCMS: m/z=409.4 [M+H]⁺; ¹H NMR (400 MHz, DMSO-d₆) δ 12.13 (s, 1H), 11.66 (s, 1H), 8.39 (s, 1H), 5.24 (d, J=9.6 Hz, 1H), 4.36-4.23 (m, 3H), 3.99-3.88 (m, 2H), 3.27 (s, 4H), 2.78 (hept, J=6.8 Hz, 1H), 1.61 (s, 3H), 1.35 (s, 3H), 1.12 (d, J=6.8 Hz, 6H).

Preparation of 30: A solution of 29 (5.8 g, 14.1 mmol) was added into a mixed solvent of HCOOH (54.0 mL) and H₂O (6.0 mL) at r.t. Then reaction mixture was stirred for 1 h at r.t. TLC and LC-MS showed the raw material was consumed. Concentrated the reaction solution by vacuum at r.t. to get compound 30 (5.2 g, 14.0 mmol, 98.0% yield), which was used for next step directly. ESI-LCMS: m/z=368.4 [M+H]⁺; ¹H NMR (400 MHz, DMSO-d₆) δ 12.13 (s, 1H), 11.72 (s, 1H), 8.30 (s, 1H), 8.14 (s, 2H), 5.19 (d, J=9.2 Hz, 1H), 3.93 (t, J=9.2 Hz, 1H), 3.85 (dd, J=12.4, 1.9 Hz, 1H), 3.77 (d, J=3.7 Hz, 1H), 3.69-3.62 (m, 2H), 3.20 (s, 3H), 2.79 (h, J=6.8 Hz, 1H), 1.13 (dd, J=6.9, 1.2 Hz, 6H).

Preparation of 31: To a solution of 30 (5.2 g, 14.0 mmol) in dioxane (90.0 mL) and H₂O (30.0 mL) was added NaIO₄ (3.7 g, 15.4 mmol) at r.t. The reaction mixture was stirred for 3 h at r.t. LC-MS showed the raw material was consumed, and the reaction solution was cooled to 0° C. Then NaBH₄ (970.0 mg, 25.2 mmol) was added to the reaction mixture, and the raw material was consumed after 3 h by LC-MS. The reaction liquid was quenched with ammonium chloride, and adjusted the pH to 6-7 with 1N HCl, the mixture solution was concentrated to get the crude, which was purified by c.c. (DCM:MeOH=100:1-30:1) to get compound 31 (4.0 g, 68.6% yield) as a white solid. ESI-LCMS: m/z=370.4 [M+H]⁺; ¹H NMR (400 MHz, DMSO-d₆) δ 11.91 (d, J=151.0 Hz, 2H), 8.62-8.51 (m, 1H), 8.18 (s, 1H), 7.44-7.33 (m, 1H), 5.62 (d, J=7.9 Hz, 1H), 4.84 (t, J=5.7 Hz, 1H), 4.65 (d, J=5.2 Hz, 1H), 3.84 (dd, J=7.7, 3.5 Hz, 1H), 3.76 (ddd, J=12.1, 4.7, 2.7 Hz, 1H), 3.60 (ddd, J=12.0, 5.8, 3.6 Hz, 1H), 3.46 (d, J=8.8 Hz, 2H), 3.16 (s, 3H), 2.77 (h, J=6.8 Hz, 1H), 1.12 (dd, J=6.8, 2.4 Hz, 6H);

Preparation of 32: A solution of 31 (4.0 g, 6.4 mmol) was dissolved in pyridine (100.0 mL), and the reaction mixture was replaced by N₂ over 3 times, and then DMTrCl (5.1 g, 8.9 mmol) was added to the reaction mixture at r.t. Then the reaction was stirred for 30 min, TLC and LC-MS showed raw material was consumed. The reaction liquid was added into ice-water, and extracted product with EA. The organic phase was washed with brine, and dried the organic phase over Na₂SO₄, and concentrated to get crude, which was purified by c.c. (DCM:MeOH=100:1˜30:1) and SFC to get compound 32 (2.7 g, 37.1% yield) as a white solid. ESI-LCMS: m/z=672.7 [M+H]⁺; ¹H NMR (400 MHz, DMSO-d₆) δ 11.50 (s, 2H), 8.22 (s, 1H), 7.32-7.24 (m, 4H), 7.22-7.12 (m, 5H), 6.84 (dd, J=9.0, 2.4 Hz, 4H), 5.63 (d, J=7.9 Hz, 1H), 4.85 (t, J=5.6 Hz, 1H), 3.95 (dt, J=7.4, 3.3 Hz, 1H), 3.85-3.77 (m, 1H), 3.73 (s, 7H), 3.65-3.57 (m, 1H), 3.43 (ddt, J=9.9, 6.9, 3.4 Hz, 1H), 3.05 (ddd, J=10.0, 6.2, 3.3 Hz, 1H), 2.96 (ddd, J=10.0, 5.6, 3.4 Hz, 1H), 2.78 (p, J=6.8 Hz, 1H), 1.11 (d, J=6.7 Hz, 6H).

Preparation of 33: To a solution of 32 (2.7 g, 2.4 mmol) in DCM (35.0 mL) was added DCI (390.0 mg, 2.0 mmol) at r.t. Then CEP [N(iPr)₂]₂ (1.2 g, 2.5 mmol) was added to the reaction mixture, then reaction mixture was stirred for 30 min at r.t. LC-MS showed raw material was consumed. The reaction liquid was added to an aqueous solution of NaHCO₃ into ice-water, and extracted product with DCM, washed the organic phase with brine, and dried the organic phase over Na₂SO₄, then filtered and concentrated to give a residue, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20.0 min, the eluted product was collected at CH₃CN/H₂O (0.5% NH₄HCO₃)=100/0; Detector, UV 254 nm. This resulted in to give compound 33 (2.0 g, 56.4% yield) as a white solid. ESI-LCMS: m/z=872.3 [M+H]⁺; 1H NMR (400 MHz, DMSO-d₆) δ 11.79 (s, 2H), 8.23 (d, J=1.7 Hz, 1H), 7.35-7.07 (m, 9H), 6.92-6.75 (m, 4H), 5.52 (d, J=8.0 Hz, 1H), 4.21 (s, 1H), 4.10-3.99 (m, 1H), 3.84-3.65 (m, 1OH), 3.63-3.52 (m, 2H), 3.45 (ddd, J=10.2, 6.7, 3.6 Hz, 1H), 3.34 (s, 1H), 3.22 (s, 3H), 3.07 (ddd, J=10.2, 6.4, 3.4 Hz, 1H), 2.97 (ddd, J=10.0, 5.6, 3.5 Hz, 1H), 2.78 (dt, J=12.2, 6.4 Hz, 3H), 1.20-1.05 (m, 18H), ³¹P NMR (162 MHz, DMSO-d₆) δ 148.20, 147.13.

### Example 49

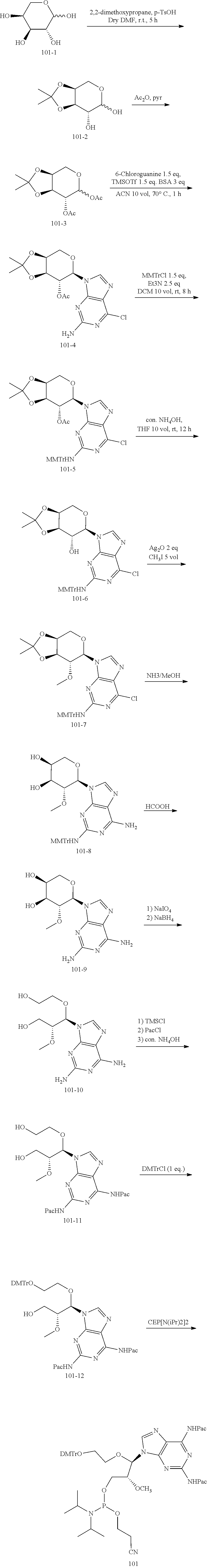

### Example 50

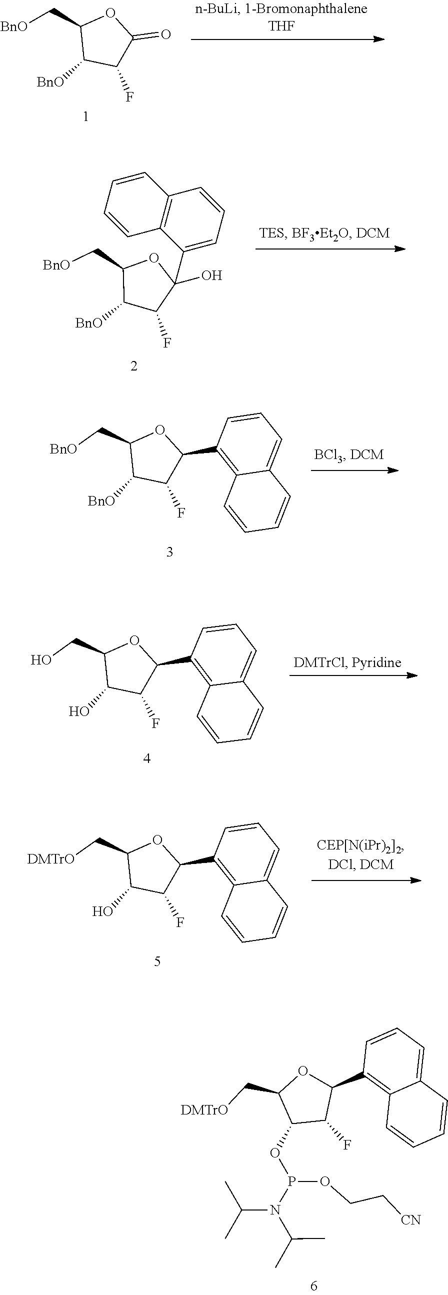

Preparation of 2: To a solution of 1-bromonaphthalene (5.2 g, 25.0 mmol) in dry THF (100.0 mL) was added n-BuLi (13.5 mL, 21.7 mmol, 1.6 M) drop wise at −78° C., then the mixture was stirred at −78° C. for 0.5 h, after that, a solution of 1 (5.5 g, 16.7 mmol) in THF (20.0 mL) was added into the mixture drop wise maintaining inner temperature below −70° C., then the reaction mixture was stirred for 1 h at −70° C. LC-MS showed 1 was consumed completely, the reaction was quenched with saturated ammonium chloride solution (80.0 mL) and extracted with EA, The organic layer was washed with brine, dried over Na₂SO₄, and concentrated under reduced pressure to give a residue, which was purified by Flash-Prep-HPLC with the following conditions (IntelFlash-1): Column, Cis silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 2 (5.8 g, 76.3% yield) as a white solid. ESI-LCMS: m/z 441 [M-OH].

Preparation of 3: To the solution of 2 (5.8 g, 12.6 mmol) in DCM (100.0 mL) was added TES (1.7 g, 14.7 mmol) at −78° C., BF₃. Et₂O (2.7 g, 18.9 mmol) was added into the mixture drop-wise at −78° C. The mixture was stirred at −40° C. for 1 h. LC-MS showed 2 was consumed completely, the solution was added into a saturated sodium bicarbonate solution (50.0 mL) and extracted with DCM. The organic layer was washed with brine, dried over Na₂SO₄, and concentrated under reduced pressure to give a residue, which was purified by Flash-Prep-HPLC with the following conditions (IntelFlash-1): Column, Cis silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=7/3; Detector, UV 254 nm. This resulted in to give 3 (2.7 g, 48.2%) as a white solid. ESI-LCMS: m/z 460 [M+H₂O]⁺; ¹H-NMR (600 MHz, CDCl₃): δ 8.01-8.00 (d, J=6.5 Hz, 1H), 7.88-7.87 (d, J=7.6 Hz, 2H), 7.77-7.76 (d, J=8.2 Hz, 1H), 7.56-7.49 (m, 2H), 7.38-7.23 (m, 11H), 6.98-5.94 (d, J=26.9 Hz, 1H), 5.09-4.99 (dd, J=61.1 Hz, 1H), 4.71-4.69 (d, J=11.6 Hz, 1H), 4.66-4.59 (m, 2H), 4.43-4.41 (d, J=11.6 Hz, 2H), 4.14-4.08 (m, 1H), 4.02-4.00 (dd, J=13.4 Hz, 1H), 3.81-3.78 (dd, J=14.8 Hz, 1H); ¹⁹F-NMR (CDCl₃): δ −193.24.

Preparation of 4: To a solution of 3 (2.7 g, 6.0 mmol) in dry DCM (40.0 mL) was added BCl₃ (36.0 mL, 36.0 mmol, 1 M) drop wise at −78° C., and the reaction mixture was stirred at −78° C. for 0.5 h. LC-MS showed 3 was consumed completely. After completion of reaction, the resulting mixture was quenched with MeOH (20.0 mL), then neutralized with sodium hydroxide solution (40.0 mL, 2 M). The mixture was extracted with DCM and concentrated to give a crude, the crude was dissolved in MeOH (30.0 mL) and added a sodium hydroxide solution (30.0 mL, 4 M), and the mixture was stirred at r.t. for 30 min. The mixture was extracted with EA, the organic layer was washed with brine, dried over Na₂SO₄, and concentrated under reduced pressure to give a residue, which was purified by silica gel column chromatography (DCM:MeOH=40:1˜15:1) to give 4 (1.3 g, 81.2%) as a white solid. ESI-LCMS: m/z 261 [M−H]⁻; ¹H-NMR (DMSO-d₆): δ 7.98-7.97 (d, J=10.2 Hz, 2H), 7.89-7.87 (m, 2H), 7.63-7.49 (m, 3H), 5.80-5.76 (d, J=26.3 Hz, 1H), 5.43 (s, 1H), 5.00 (s, 1H), 4.85-4.76 (d, J=58.4 Hz, 1H), 4.03-3.85 (m, 3H), 3.68-3.66 (m, 1H), 3.65-3.53 (m, 1H); ¹⁹F-NMR (DMSO-d₆): δ −192.76.

Preparation of 5: To a solution of 4 (1.3 g, 5.0 mmol) in pyridine (20.0 mL) was added DMTrCl (6.1 g, 16.0 mmol) at r.t. The reaction mixture was stirred at r.t. for 1 h. The LC-MS showed 4 was consumed and water (100.0 mL) was added. The product was extracted with EA and the organic layer was washed with brine and dried over Na₂SO₄, concentrated to give the crude, which was further purified by silica gel (EA: PE=1:30˜1:10) to give 5 (2.2 g, 78.5%) as a yellow solid. ESI-LCMS: m/z 563 [M−H]⁻; ¹H-NMR (600 MHz, DMSO-d₆): δ 8.03-7.99 (m, 2H), 7.91-7.86 (m, 2H), 7.64-7.57 (m, 2H), 7.49-7.48 (d, J=6.8 Hz, 2H), 7.40-7.24 (m, 8H), 6.89-6.88 (m, 4H), 5.92-5.88 (d, J=26.6 Hz, 1H), 5.50-5.49 (d, J=4.5 Hz, 1H), 4.96-4.87 (d, J=56.2 Hz, 1H), 4.18-4.14 (m, 2H), 3.74 (s, 6H), 3.42-3.40 (d, J=9.9 Hz, 1H), 3.33 (m, 2H); ¹⁹F-NMR (DMSO-d₆): δ −192.18.

Preparation of 6: To a suspension of 5 (2.2 g, 3.9 mmol) in DCM (20.0 mL) was added DCI (391.0 mg, 3.3 mmol) and CEP[N(iPr)₂]₂ (1.4 g, 4.7 mmol). The mixture was stirred at r.t. for 1 h. The LC-MS showed 5 was consumed completely. The solution was washed with a saturated sodium bicarbonate solution and brine successively, dried over Na₂SO₄, concentrated to give the crude, which was purified by Flash-Prep-HPLC with the following conditions (IntelFlash-1): Column, Cis silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (2.5 g, 83.8%) as a white solid. ESI-LCMS: m/z 765 [M+H]⁺; ¹H-NMR (400 MHz, DMSO-d₆): δ 8.07-7.86 (m, 4H), 7.64-7.56 (m, 2H), 7.49-7.45 (m, 2H), 7.41-7.21 (m, 8H), 6.89-6.84 (m, 4H), 6.02-5.93 (m, 1H), 5.19-4.98 (m, 1H), 4.61-4.34 (m, 1H), 4.26-4.24 (m, 1H), 3.74-3.73 (m, 6H), 3.70-3.61 (m, 1H), 3.57-3.42 (m, 4H), 3.29-3.24 (m, 1H), 2.67-2.64 (m, 1H), 2.56-2.52 (m, 1H), 1.09-1.04 (m, 1H), 0.98-0.97 (d, J=6.7 Hz, 3H), 0.89-0.87 (d, J=6.7 Hz, 3H); ¹⁹F-NMR (DMSO-d₆): δ −191.75, −191.76, −191.84, −191.85; ³¹P-NMR (DMSO-d₆): δ 149.51, 149.47, 149.16, 149.14.

### Example 51

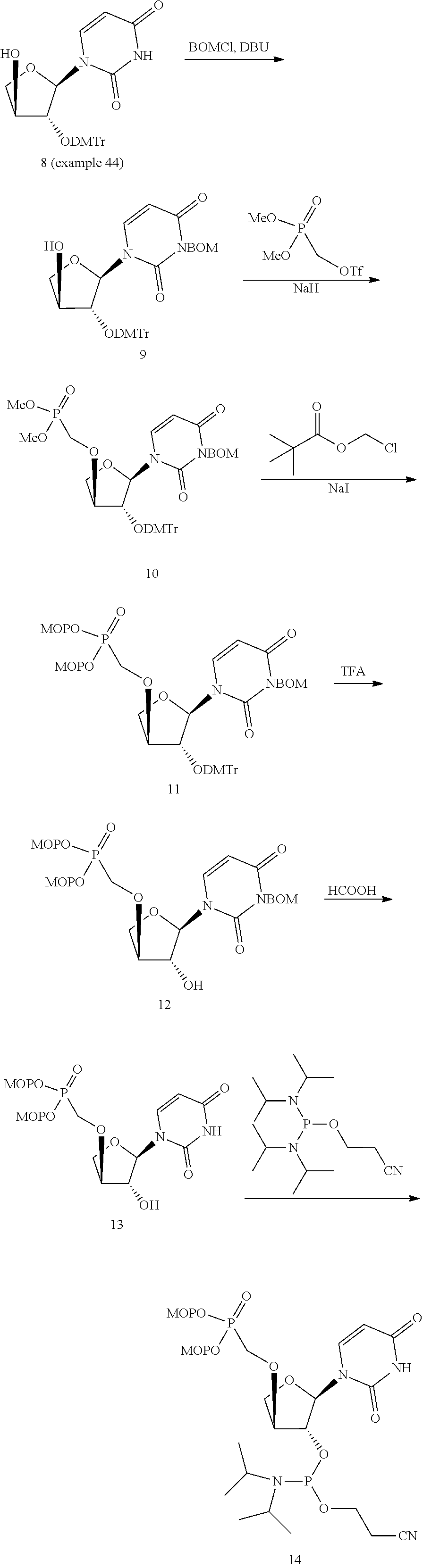

### Preparation of 9

To a solution of 8 (from Example 44) (6.6 g, 10.86 mmol, 85% purity, 1 eq) and DBU (3.31 g, 21.72 mmol, 3.27 mL, 2 eq) in DMF (70 mL) was added BOMCl (2.55 g, 16.29 mmol, 2.26 mL, 1.5 eq) at 0° C. The mixture was stirred at 20° C. for 12 h. The mixture was diluted with EtOAc (180 mL) and washed with H₂O (80 mL*3), and brine (80 mL), dried over Na₂SO₄, filtered and concentrated under reduced pressure to give a residue. The residue was purified by flash silica gel chromatography (ISCO®; 80 g SepaFlash® Silica Flash Column, Eluent of 10-60%, EtOAc/PE gradient @60 mL/min) to give 9 (5.2 g, 70% yield,) as a white foam. LCMS (ESI): m/z 659.1.; ¹H NMR (400 MHz, DMSO-d₆) δ=7.63 (d, J=8.3 Hz, 1H), 7.40-7.15 (m, 14H), 6.85 (t, J=8.0 Hz, 4H), 5.97 (s, 1H), 5.75 (d, J=8.0 Hz, 1H), 5.39-5.26 (m, 2H), 5.24 (d, J=2.0 Hz, 1H), 4.61 (s, 2H), 3.97 (s, 1H), 3.94-3.83 (m, 2H), 3.68 (d, J=10.0 Hz, 6H), 3.38 (s, 1H)

### Preparation of 10

To a solution of 9 (5.2 g, 8.17 mmol, 1 eq) and dimethoxyphosphorylmethyl trifluoromethanesulfonate (6.67 g, 24.50 mmol, 3 eq) in THF (50 mL) was added NaH (816.65 mg, 20.42 mmol, 60% purity, 2.5 eq) at −5° C. The mixture was stirred at 0° C. for 0.5 h. The reaction mixture was quenched by addition H₂O (50 mL) and diluted with EtOAc (100 mL), then washed with H₂O (50 mL), brine (50 mL), the organic layer was dried over Na₂SO₄, filtered and concentrated under reduced pressure. The residue was purified by flash silica gel chromatography (ISCO®; 80 g SepaFlash® Silica Flash Column, Eluent of 0-50%, EtOAc/DCM gradient @60 mL/min) to give 10 (4.2 g, 66.42% yield,) as a white foam. LCMS (ESI): m/z 781.1 [M+Na]⁺, ¹H NMR (400 MHz, CDCl₃) δ=7.49-7.25 (m, 14H), 7.21-7.15 (m, 1H), 6.82 (d, J=8.8 Hz, 4H), 6.46 (s, 1H), 5.65 (d, J=8.2 Hz, 1H), 5.57-5.39 (m, 2H), 4.72 (s, 2H), 4.16-4.07 (m, 2H), 3.93 (dd, J=2.6, 10.8 Hz, 1H), 3.81-3.59 (m, 11H), 3.81-3.59 (m, 1H), 3.24 (dd, J=10.6, 13.5 Hz, 1H), 3.10 (dd, J=9.8, 13.3 Hz, 1H), 2.79 (d, J=2.2 Hz, 1H); ³¹P NMR (CD₃CN) δ=22.37 (s)

### Preparation of 11

To a solution of 10 (4.6 g, 6.06 mmol, 1 eq) and NaI (2.73 g, 18.19 mmol, 3 eq) in MeCN (15 mL) was added chloromethyl 2,2-dimethylpropanoate (3.65 g, 24.25 mmol, 3.51 mL, 4 eq). The mixture was stirred at 85° C. for 24 h. The reaction mixture was concentrated under reduced pressure to give a residue. The residue was purified by flash silica gel chromatography (ISCO®; 40 g SepaFlash® Silica Flash Column, Eluent of 0-50%, EtOAc/PE gradient @40 mL/min) to give 11 (2.7 g, 44.6% yield) as a pale yellow solid. LCMS (m/z): 981.1 [M+Na]⁺.

### Preparation of 12

To a solution of 11 (2.7 g, 2.82 mmol, 1 eq) in DCM (20 mL) was added Et₃S¹H (645.45 mg, 2.82 mmol, 5 mL, 1 eq), followed by addition of TFA (1.54 g, 13.51 mmol, 1 mL, 4.80 eq). The mixture was stirred at 20° C. for 0.5 h. The reaction mixture was concentrated under reduced pressure to give a residue. The residue was purified by flash silica gel chromatography (ISCO®; 24 g SepaFlash® Silica Flash Column, Eluent of 0-50%, EtOAc/DCM gradient @30 mL/min) to give 12 (1.6 g, 84.82% yield,) as a pale yellow solid. LCMS (ESI): m/z 679.1 [M+Na]⁺; ¹H NMR (400 MHz, CDCl₃) δ=7.44 (d, J=8.2 Hz, 1H), 7.38-7.26 (m, 5H), 5.76 (d, J=8.2 Hz, 1H), 5.69-5.62 (m, 4H), 5.51-5.43 (m, 1H), 5.51-5.43 (m, 1H), 4.70 (s, 2H), 4.30 (s, 1H), 4.26-4.06 (m, 4H), 3.90 (dd, J=4.9, 8.4 Hz, 2H), 3.22-3.06 (m, 1H), 1.22 (s, 18H); ³¹P NMR (162 MHz, CD₃CN) δ=20.25 (s, 1P).

### Preparation of 13

To a mixture of 12 (1.4 g, 2.13 mmol, 1 eq) in isopropanol (20 ml) and H₂O (2 mL) added Pd/C (1.4 g,) and HCOOH (51.22 mg, 1.07 mmol, 2 mL) under N₂. The suspension was degassed under vacuum and purged with H₂ several times. The mixture was stirred under H₂ (15 PSI) at 15° C. for 5 h. The reaction mixture was filtered and the filtrate was concentrated to give a residue. The residue was purified by flash silica gel chromatography (ISCO®; 24 g SepaFlash® Silica Flash Column, Eluent of 0-50%, EtOAc/DCM gradient @30 mL/min) to give 13 (848 mg, 74.14% yield) as a white foam. LCMS (ESI): m/z 537.0 [M+H]^{+; 1}H NMR (400 MHz, CDCl₃) δ=10.01 (s, 1H), 7.53 (d, J=8.0 Hz, 1H), 5.78-5.63 (m, 6H), 4.40 (s, 1H), 4.35-4.22 (m, 3H), 4.11 (d, J=1.5 Hz, 1H), 3.88 (d, J=8.5 Hz, 2H), 1.22 (s, 18H); ³¹P NMR (162 MHz, CD₃CN) δ=20.17 (s, 1P.)

### Preparation of 14

To a solution of 13 (848 mg, 1.58 mmol, 1 eq) in DCM (10 mL) was added 3-bis(diisopropylamino)phosphanyloxypropanenitrile (571.73 mg, 1.90 mmol, 602.45 uL, 1.2 eq) at 0° C., followed by addition of 1H-imidazole-4,5-dicarbonitrile (186.7 mg, 1.58 mmol, 1 eq). The mixture was stirred at 15° C. for 1 h. The reaction mixture was quenched by addition of sat. aq·NaHCO₃ (10 mL) and diluted with DCM (20 mL). Then the organic layer was washed with sat. aq·NaHCO₃ (10 mL*2), dried over Na₂SO₄, filtered and concentrated under reduced pressure. The residue was purified by flash silica gel chromatography (ISCO®; 12 g SepaFlash® Silica Flash Column, Eluent of 0-50%, phase A: PE with 0.5% TEA; phase B: EA with 10% EtOH, 30 mL/min) to give 14 (720 mg, 61.21% yield,) as a colorless oil. LCMS (ESI): m/z 737.1 [M+H]^{+; 1}H NMR (400 MHz, CD₃CN) δ=9.17 (s, 1H), 7.49 (d, J=8.0 Hz, 1H), 5.91-5.77 (m, 1H), 5.65-5.54 (m, 5H), 4.49-4.26 (m, 2H), 4.23-4.07 (m, 2H), 3.92-3.55 (m, 6H), 2.71-2.61 (m, 2H), 1.24-1.16 (m, 30H); ³¹P NMR (162 MHz, CD₃CN) δ=151.59.

### Example 52: Synthesis of 102

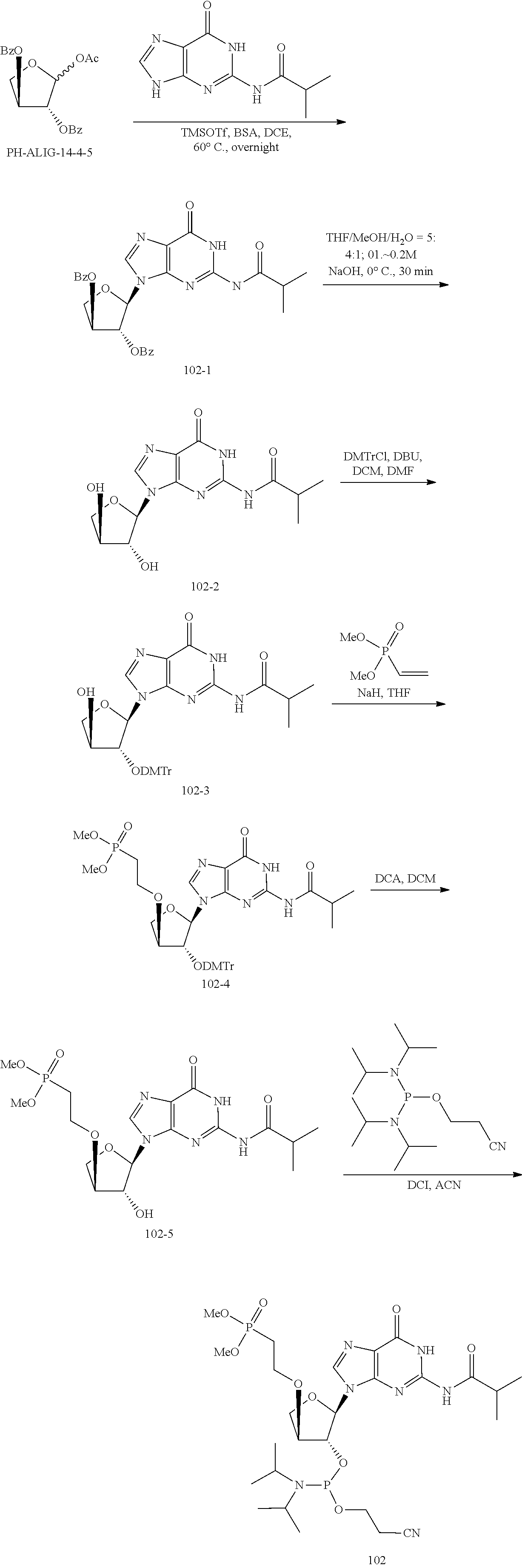

### Example 53: Synthesis of 103

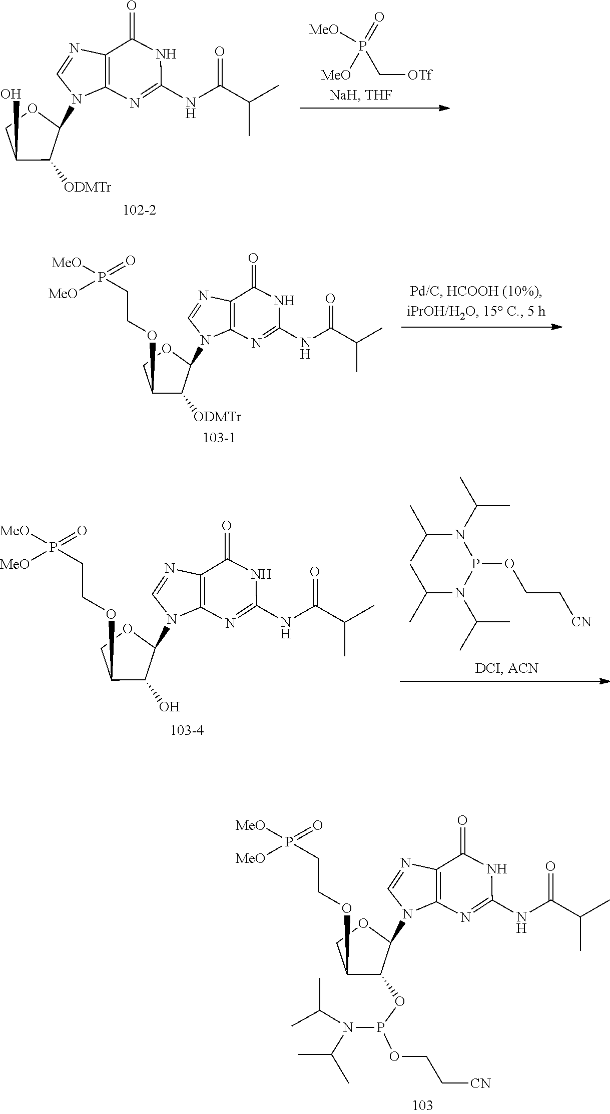

### Example 54: Synthesis of 104

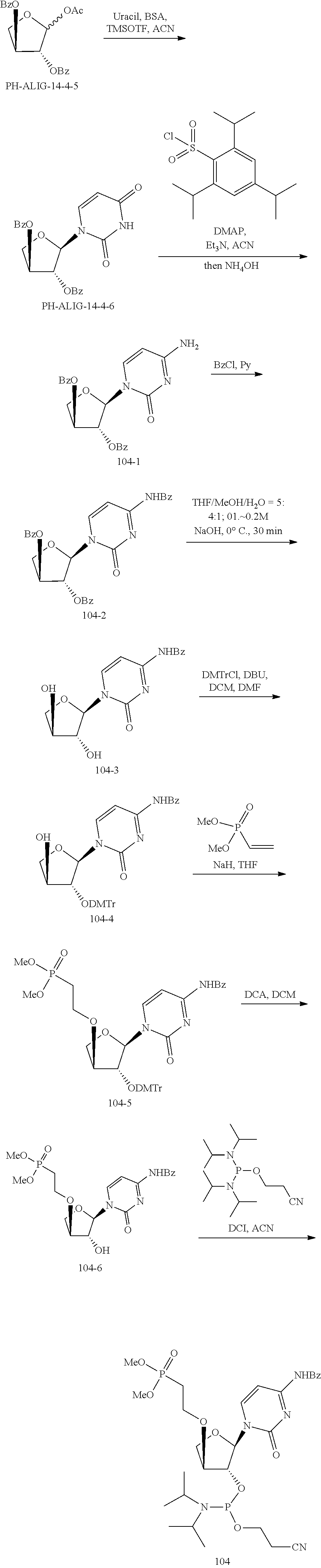

### Example 55: Synthesis of 105

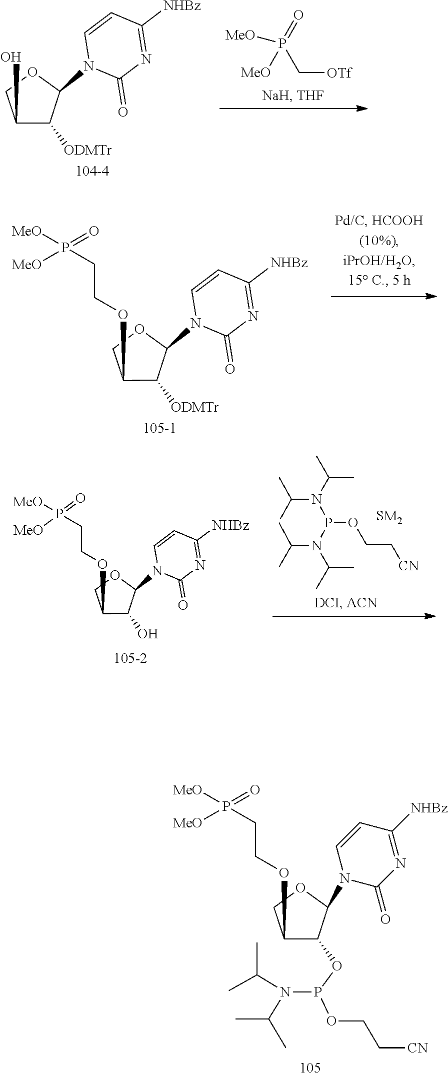

### Example 56

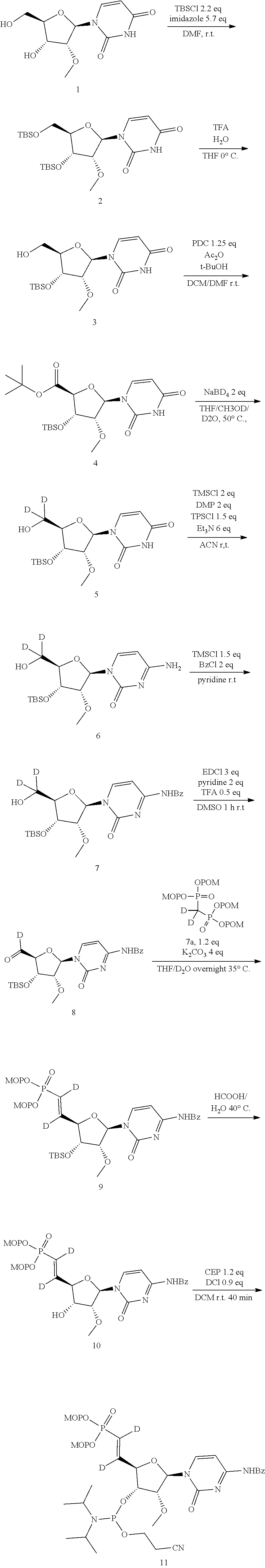

Preparation of 2: A 2 L three-necked round bottom flask equipped with magnetic stirrer and thermometer was charged with 1 (60.0 g, 228.8 mmol) in dry DMF (600.0 mL) at r.t., imidazole (95.2 g, 1.3 mol) was added into the mixture reaction, then the reaction mixture was cooled down to turn 5° C., TBSCl (76.8 g, 499.3 mmol) was added into the mixture reaction, the reaction mixture was allowed to stir for 12 h at r.t. 1 was consumed by LCMS, then the reaction mixture was added in the saturated sodium bicarbonate solution (1.0 L), after quenching the reaction, the aqueous layer was extracted with EA (400.0 mL*2), the combined organic layer was washed with saturated brine and dried over anhydrous sodium sulfate, the organic layer was concentrated to get crude 2 (110.2 g, 212.8 mmol, 93.1% yield) as a white solid, the crude product was used directly for the next step without purification. ESI-LCMS: m/z=487.3 [M+H]⁺.

Preparation of 3: A 3 L three-necked round bottom flask equipped with magnetic stirrer and thermometer was charged with 2 (117.0 g, 225.9 mmol) in THF (550.0 mL) at r.t., water (275.0 mL) was added into the mixture reaction, then the reaction mixture was cooled down to turn 0° C. and add TFA (275.0 mL) by constant pressure funnel after 4 h, the reaction mixture was allowed to stir for 2 h at 0° C. 2 was consumed by TLC. Then, reaction mixture was added in a mixture solvent of ammonium hydroxide (250.0 mL) and water (800.0 mL) at 0° C., after quenching the reaction, the aqueous layer was extracted with EA (500.0 mL*2), the combined organic layer was washed with saturated brine and dried over anhydrous sodium sulfate, the organic layer was concentrated to get crude which was purified by silica gel column chromatography (PE:EA=10:1 to 0:1) to give compound 3 (51.1 g, 59.3% yield) as a white solid. ¹H-NMR (600 MHz, DMSO-d₆): δ=11.35 (s, 1H), 7.919 (d, J=6 Hz, 1H), 5.82 (s, 1H), 5.65 (d, J=6 Hz, 1H), 5.18 (s, 1H), 4.29 (s, 1H), 3.83 (s, 2H), 3.65 (d, J=12 Hz, 1H), 3.53 (d, J=6 Hz, 1H), 3.32 (d, J=6 Hz, 1H), 0.87 (s, 9H), 0.08 (s, 6H). ESI-LCMS: m/z=373.1 [M+H]⁺.

Preparation of 4: A 3 L three-necked round bottom flask equipped with magnetic stirrer and thermometer was charged with 3 (50.0 g, 131.5 mmol) in a mixture solvent of DCM (250.0 mL) and DMF (70.0 mL) at r.t., the mixture solution was cooled down to turn 5° C., PDC (63.1 g, 164.4 mmol) and t-BuOH (200.0 mL) were added into the mixture reaction, keep the reaction at 5° C. and add Ac₂O (130.0 mL) by constant pressure funnel after 0.5 h, the reaction mixture was allowed to stir for 4 h at r.t. 3 was consumed by lc-ms, then the reaction mixture was added in the saturated sodium bicarbonate (400.0 mL), after quenching the reaction, the aqueous layer was extracted with DCM (500.0 mL*2), the combined organic layer was washed with saturated brine and dried over anhydrous sodium sulfate, the organic layer was concentrated to get crude which was purified by silica gel column chromatography (PE:EA=10:1 to 2:1) to give compound 4 (29.8 g, 50.6% yield) as a white solid. ¹H-NMR (DMSO d₆): δ=11.42 (s, 1H), 8.04 (d, J=6 Hz, 1H), 5.82 (s, 1H), 5.78 (d, J=6 Hz, 1H), 4.44 (s, 1H), 4.25 (s, 1H), 3.84 (s, 1H), 3.32 (s, 3H), 1.46 (s, 9H), 0.89 (s, 9H), 0.12 (s, 6H). ESI-LCMS: m/z=443.1 [M+H]⁺.

Preparation of 5: To a solution of 4 (33.0 g, 74.7 mmol) in dry THF (330.0 mL) was added CH₃OD (66.0 mL) and D₂O (33.0 mL) at r.t. Then the reaction mixture was added NaBD₄ (9.4 g, 224.0 mmol) three times per an hour at 50° C. The solution was stirred at 50° C. for 3 h. LCMS showed 4 was consumed. Water (300.0 mL) was added. The product was extracted with EA (2*300.0 mL). The organic layer was washed with brine and dry over by Na₂SO₄. Then the solution was concentrated under reduced pressure, crude was purified by silica gel column chromatography (PE:EA=10:1 to 3:1) to give 5 (19.1 g, 68.5% yield) as a white solid. ¹H-NMR (600 MHz, DMSO d₆): δ=11.35 (s, 1H), 7.92-7.91 (d, J=6 Hz, 1H), 5.83-5.82 (d, J=6 Hz, 1H), 5.66-5.65 (d, J=6 Hz, 1H), 5.14 (s, 1H), 4.30-4.28 (m, 1H), 3.84-3.82 (m, 2H), 3.34 (s, 3H), 0.88 (s, 9H), 0.09 (s, 6H). ESI-LCMS: m/z 375 [M+H]⁺.

Preparation of 6: To a solution of 5 (19.1 g, 51.1 mmol) in dry ACN (190.0 mL) was added Et₃N (20.7 g, 204.6 mmol) at r.t. and TMSCl (11.1 g, 102.1 mmol) at 0° C. Then the reaction mixture was stirred at r.t. for 40 min. LCMS showed 5 was consumed and an intermediate was formed. Then the solution was added DMAP (12.5 g, 102.3 mmol), Et₃N (10.3 g, 102.1 mmol) and TPSCl (23.2 g, 76.6 mmol). The reaction mixture was stirred at r.t. for 15 h. LCMS showed the intermediate was consumed and conformed another intermediate. Then was added NH₄OH (200.0 mL) and stirred at r.t. for 24 h to give the mixture of product. The product was extracted with EA (2*200.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/2 increasing to CH₃CN/H₂O=1/0 within 20 min, the eluted product was collected at CH₃CN/H₂O=1/0; Detector, UV 254 nm. This resulted in to give 6 (14.0 g, 73.7% yield). ¹H-NMR (DMSO-d₆): δ=7.89-7.88 (d, J=6 Hz, 1H), 7.20-7.18 (d, J=12 Hz, 2H), 5.85-5.84 (d, J=6 Hz, 1H), 5.73-5.72 (d, J=6 Hz, 1H), 5.09 (s, 1H), 4.24-4.23 (m, 1H), 3.81-3.80 (d, J=6 Hz, 1H), 3.69-3.68 (m, 1H), 3.36 (s, 3H), 0.87 (s, 9H), 0.07 (s, 6H). ESI-LCMS: m/z 374 [M+H]⁺.

Preparation of 7: To a solution of 6 (14.0 g, 37.5 mmol) in pyridine (140.0 mL) was added TMSCl (6.3 g, 58.0 mmol) at 0° C. and the mixture was stirred at r.t. for 1.5 h. LCMS showed 6 was consumed and an intermediate(a) was formed. Then was added BzCl (10.8 g, 76.8 mmol) at 0° C. and the mixture was stirred at r.t. for 1.5 h. LCMS showed the intermediate was consumed and another intermediate was formed. Then the mixture was added NH₄OH (30.0 mL) and was stirred at r.t. for 15 h. LCMS showed the intermediate was consumed. Water (300.0 mL) was added. The solution was extracted with EA (2*200.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=1/0; Detector, UV 254 nm. This resulted in to give 7 (10.5 g, 58.6% yield). ¹H-NMR (600 MHz, DMSO d₆): δ=11.29 (s, 1H), 8.53-8.52 (d, J=6 Hz, 1H), 8.01-8.00 (d, J=6 Hz, 2H), 7.63-7.61 (m, 1H), 7.52-7.50 (m, 2H), 7.36 (s, 1H), 5.88 (s, 1H), 5.24 (s, 1H), 4.28-4.26 (m, 1H), 3.91 (s, 1H), 3.81-3.79 (m, 1H), 3.46 (s, 3H), 0.87 (s, 9H), 0.08 (s, 6H). ESI-LCMS: m/z 478 [M+H]⁺.

Preparation of 8: To a solution of 7 (10.5 g, 22.0 mmol) in DMSO (105.0 mL) was added EDCI (12.7 g, 66.0 mmol), dry pyridine (1.7 g, 22.0 mmol) at r.t. and TFA (1.3 g, 11.0 mmol) at 0° C. Then the reaction mixture was stirred for 1 h. LCMS showed 7 was consumed. Water (100.0 mL) was added. The solution was extracted with EA (2*200.0 mL). The organic layer was washed with brine and dry over by Na₂SO₄. Then the solution was concentrated under reduced pressure to give the crude product 8 which was used in next step directly. ESI-LCMS: m/z 475 [M+H]⁺.

Preparation of 9: To a solution of 8 in dry THF (120.0 mL) and D₂O (40.0 mL) was added K2CO3 (12.2 g, 88.1 mmol) and 7a (16.8 g, 26.5 mmol) then the reaction mixture was stirred for 15 h at 35° C. under the N₂ atmosphere. LCMS showed 95% 7 was consumed. Water (60.0 mL) was added. The solution was extracted with EA (2*150.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=4/1; Detector, UV 254 nm. This resulted in to give 9 (9.3 g, 54.1% yield). ¹H-NMR (DMSO-d₆) δ=11.33 (s, 1H), 8.17-8.15 (d, J=12, 1H), 8.02-8.00 (d, J=12, 1H), 7.64-7.62 (m, 1H), 7.53-7.50 (m, 2H), 7.44-7.42 (d, J=12, 1H), 4.46-4.44 (d, J=12, 1H), 4.24-4.23 (d, J=6, 1H), 3.93-3.91 (d, J=12, 1H), 1.16 (s, 18H), 0.86 (s, 9H)), 0.08-0.06 (d, J=12, 6H). ESI-LCMS: m/z 782 [M+H]⁺. ³¹P-NMR (DMSO-d₆) δ=16.77, 16.00.

Preparation of 10: 9 (9.3 g, 11.9 mmol) in the mixture solution of HOAc (140.0 mL) and H₂O (140.0 mL) was stirred at 30° C. for 15 h. LCMS showed 9 was consumed. The solution was added in the ice water and extracted with EA (2*300.0 mL). The organic layer was quenched to pH=6-7 and then washed with brine and dry over Na₂SO₄. Then the solution was concentrated under reduced, crude was purified by pressure Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=2.5/1; Detector, UV 254 nm. This resulted in to give 10 (5.1 g, 64.6% yield). ¹H-NMR (DMSO-d₆) δ=9.09 (s, 1H), 7.92-7.85 (m, 3H), 7.60-7.48 (m, 4H), 6.02 (s, 1H), 5.71-5.64 (m, 4H), 4.53-4.51 (m, 1H), 3.94-3.70 (m, 5H), 3.31 (s, 1H), 1.21 (s, 18H). ³¹P-NMR (DMSO-d₆) δ=16.45. ESI-LCMS: m/z 668 [M+H]⁺.

Preparation of 11: To a suspension of 10 (4.6 g, 6.9 mmol) in DCM (45.0 mL) added CEOP[N(ipr)₂]₂ (2.5 g, 8.3 mmol), DCI (730.4 mg, 6.2 mmol). The mixture was stirred at r.t. for 1 h. LCMS showed 10 was consumed completely. The solution was quenched by water (40.0 mL), washed with brine (2*20.0 mL) and dry over by Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=4/1; Detector, UV 254 nm. This resulted in to give 11 (4.7 g, 5.4 mmol, 78.3% yield) as a white solid. ¹H-NMR (600 MHz, DMSO-d₆) δ=11.34 (s, 1H), 8.18-8.16 (m, 1H), 8.02-8.01 (d, J=6, 2H), 7.65-7.42 (m, 4H), 5.95-5.93 (m, 1H), 5.66-5.61 (m, 4H), 4.64-4.57 (m, 1H), 4.32-4.31 (d, J=6, 1H), 4.12-4.10 (m, 1H), 3.81-3.45 (m, 7H), 2.81-2.79 (m, 2H), 1.16-1.13 (m, 30H). ³¹P-NMR (CDCl₃-d₆) 6=150.65, 150.20, 16.64, 15.41. ESI-LCMS: m/z 868 [M+H]⁺.

### Example 57

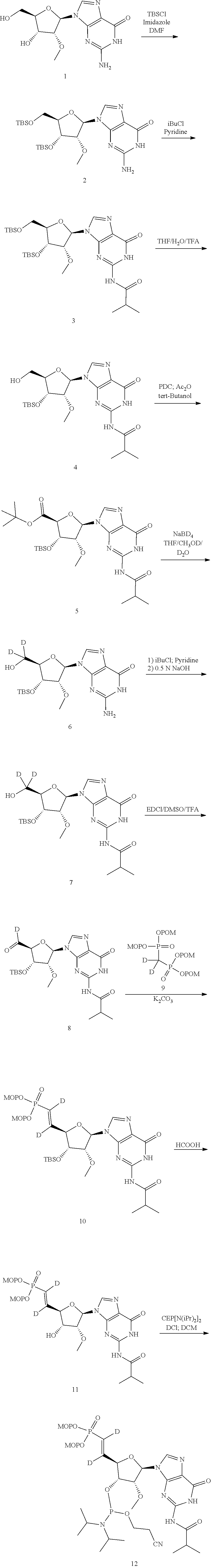

### Example 57 Scheme

Preparation of 2: 1 (94.5 g, 317.9 mmol) was dissolved in dry DMF (1000 mL) under N₂ atmosphere. To the solution TBSCl (119.3 g, 794.7 mmol) and imidazole (75.8 g, 1.1 mol) was added at 25° C. and stirred for 17 hr. LCMS showed all of 1 consumed. The reaction mixture was washed with H₂O (3000*2 mL), EA (2000*2 mL) and brine (1500 mL). Dried over Na₂SO₄ and concentrated to give crude which goes to the next step. The reaction mixture was concentrated to give crude 2 (200 g, crude). ESI-LCMS: m/z 526 [M+H]⁺.

Preparation of 3: 2 (175.1 g, 333.0 mmol) was evaporated with pyridine and dried in vacuo for two times. The residue was dissolved in pyridine (1500 mL) under N₂. To the solution, i-BuCl (88.7 g, 832.6 mmol) was added at 5° C. under N₂ atmosphere and stirred for 3 hr. LCMS showed all of 2 consumed. The reaction mixture was washed with H₂O (3000*2 mL), EA (2000*2 mL) and brine (1500 mL). Dried over Na₂SO₄ and concentrated to give crude which goes to the next step. The reaction mixture was concentrated to give crude 3 (228 g, crude). ESI-LCMS: m/z 596 [M+H]⁺.

Preparation of 4: A solution of 3 (225 g, 377.6 mmol) was in THF (2000 mL) was added H₂O (500 mL) and TFA (500 mL) was added at 5° C. Then the reaction mixture was stirred at 5° C. for 1 hr. LCMS showed all of 3 consumed. Con NH₄OH (aq) was added to mixture to quench the reaction until the pH=7-8, then washed with H₂O (2000*2 mL), EA (2000*2 mL) and brine (1500 mL). Dried over Na₂SO₄ and concentrated to give crude which was purified by cc. The reaction mixture was concentrated to give 4 (155.6 g, 83.9% yield). ESI-LCMS: m/z 482 [M+H]⁺.

Preparation of 5: 4 (100 g, 207.6 mmol) was dissolved in dry DMF (1000 mL) under N₂. To the solution, t-BuOH (307.8 g, 4.2 mol), PDC (156.1 g, 0.4 mol) and Ac₂O (212.0 g, 2.1 mol) was added at 25° C. under N₂ atmosphere and stirred at 25° C. for 2 hr. LCMS and TLC showed all of 4 consumed. NaHCO₃ (aq) was added to mixture to quench the reaction until the pH=7-8, then washed with H₂O (500*2 mL), EA (500*2 mL) and brine (500 mL). Dried over Na₂SO₄ and concentrated to give crude which was purified by cc. and MPLC. The reaction mixture was concentrated to give 5 (77.3 g, 61.6% yield,). ESI-LCMS: m/z 552 [M−H]⁺.

Preparation of 6: 5 (40.0 g, 72.6 mmol) was dissolved in dry THF (400 mL) under N₂. To the solution, MeOD (80 mL) and D₂O (40 mL) was added at 25° C. under N₂ atmosphere, then NaBD₄ (9.1 g, 217.4 mmol) was added for three times and stirred for 15 hr. LCMS and TLC showed all of 5 consumed. The mixture was concentrated to give crude which goes to the next step. The reaction mixture was concentrated to give crude 6 (30 g, crude). ESI-LCMS: m/z 414 [M+H]⁺

Preparation of 7: 6 (30 g, crude) was evaporated with pyridine and dried in vacuo for two times. The residue was dissolved in dry pyridine (300 mL) under N₂. Then iBuCl (15.5 g, 145.3 mmol) was slowly added to the reaction mixture at 0° C. under N₂ atmosphere and stirred at 25° C. for 1 hr. LCMS and TLC showed all of 6 consumed. NaHCO₃ (aq) was added to mixture to quench the reaction until the pH=7.5, then washed with H₂O (1500 mL), EA (1000*2 mL) and brine (1500 mL). Dried over Na₂SO₄ and concentrated to give crude residue R1. NaOH (8 g, 0.2 mol), MeOH (80 mL) and H₂O (20 mL) made up NaOH (aq). The residue R1 (40 g, 3.63 mmol) was dissolved in pyridine (20 mL). To the solution, 2N NaOH (aq) (100 ml) was added to the solution and stirred the reaction 15 min at 5° C. TLC showed all of R1 consumed. The mixture was added NH₄C1 to pH=7-8 at 5° C., and concentrated to give crude which was purified by cc. The product was concentrated to give 7 (15.5 g, 33.00% yield over two steps,). ESI-LCMS: m/z 484[M+H]⁺.

Preparation of 8: To a stirred solution of 7 (15.5 g, 32.1 mmol) in DMSO (150 mL) were added EDCI (18.5 g, 96.3 mmol), pyridine (2.5 g, 32.1 mmol), TFA (1.8 g, 16.0 mmol) at room temperature under N₂ atmosphere. The reaction mixture was stirred for 1 h at room temperature. The reaction was quenched with water, extracted with EA (300.0 mL), washed with brine, dried over Na₂SO₄ and evaporated under reduced pressure give a crude 8 (17.3 g, crude) which was used directly to next step. ESI-LCMS: m/z=481 [M+H]⁺.

Preparation of 10: A solution of 8 (17.3 g, crude), 9 (21.4 g, 33.7 mmol) and K2CO3 (13.3 g, 96.3 mmol) in dry THF (204 mL) and D₂O (34 mL) was stirred 5 h at 40° C. The mixture was quenched with water, extracted with EA (600.0 mL), washed with brine, dried over Na₂SO₄ and evaporated under reduced pressure. The residue was purified by silica gel (PE:EA=5:1˜1:1) to give 10 (9.3 g, 36.6% yield over 2 steps) as a white solid. ESI-LCMS m/z=787[M+H]⁺.

¹H-NMR (DMSO-d₆): δ 11.24 (s, 1H, exchanged with D₂O), 8.74 (d, J=2.7 Hz, 2H), 8.05-8.04 (d, J=7.4 Hz, 2H), 7.65 (t, 1H), 7.57-7.54 (t, 2H), 6.20 (d, J=5.0 Hz, 1H), 5.64-5.58 (m, 4H), 4.77 (t, 1H), 4.70 (t, 1H), 4.57-4.56 (t, 1H), 3.35 (s, 3H), 1.09 (d, J=6.5 Hz, 18H), 0.93 (s, 9H), 0.15 (d, J=1.8 Hz, 6H); ³¹P NMR (DMSO-d₆): δ 17.05.

Preparation of 11: To a round-bottom flask was added 10 (9.3 g, 11.5 mmol) in a mixture of H₂O (93 mL) and HCOOH (93 mL). The reaction mixture was stirred for 5 h at 50° C. and 15 h at 35° C. The mixture was extracted with EA (500.0 mL), washed with water, NaHCO₃ solution and brine successively, dried over Na₂SO₄ and evaporated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 20 min, the eluted product was collected at CH₃CN/H₂O (0.5% NH₄HCO₃)=3/2; Detector, UV 254 nm. To give product 11 (6.3 g, 78% yield). ¹H-NMR (600 MHz, DMSO-d₆): δ 12.17 (s, 1H, exchanged with D₂O), 11.51 (s, 1H), 8.28 (s, 1H), 6.02-6.03 (d, J=4.2 Hz, 1H), 5.63-5.72 (m, 5H), 4.60 (s, 1H), 4.43-4.45 (m, 2H), 3.40 (s, 1H), 3.38 (s, 1H), 2.83-2.88 (m, 1H), 1.15-1.23 (m, 24H); ³¹P NMR (DMSO-d₆) δ=17.69. ESI-LCMS m/z=674 [M+H]⁺.

Preparation of 12: To a solution of 11 (5.6 g, 8.3 mmol) in DCM (55.0 mL) was added the DCI (835 mg, 7.1 mmol), then CEP[N(ipr)₂]₂ (3.3 g, 10.8 mmol) was added. The mixture was stirred at r.t. for 1 h. The reaction mixture was washed with H₂O (50.0 mL) and brine (50.0 mL), dried over Na₂SO₄ and evaporated under pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. The product was concentrated to give 12 (6.3 g, 87% yield) as a white solid. ¹H-NMR (DMSO-d₆): δ 12.14 (s, 1H, exchanged with D₂O), 11.38 (s, 1H), 8.27-8.28 (d, J=6 Hz, 1H), 5.92-5.98 (m, 1H), 5.59-5.65 (m, 4H), 4.57-4.68 (m, 3H), 3.61-3.85 (m, 4H), 3.37 (s, 1H), 3.32 (s, 1H), 2.81-2.85 (m, 3H), 1.09-1.20 (m, 36H); ³¹P NMR (DMSO-d₆): δ 150.60, 149.97, 17.59, 17.16; ESI-LCMS m/z=874 [M+H]⁺.

### Example 58. Luciferase Reporter Assay in COS-7 Cells

All siNAs synthesized were tested for in vitro activity using a 3-point luciferase reporter assay and a subset of candidates were tested in a dose-response luciferase reporter assay.

In the psiCHECK™-2 reporter plasmid, Renilla luciferase is used as the primary reporter gene with the HSD17B13 gene (NM_178135.5) cloned downstream of its translational stop codon. A second reporter gene, firefly luciferase, is also expressed and used as a transfection control. COS-7 cells (ATCC, CRL-1651) were seeded into 96-well microplates and transfected with the reporter plasmid using Lipofectamine 3000 (Invitrogen, L3000001). The cells were then transfected with 10, 1, or 0.1 nM siNAs using Lipofectamine RNAiMAX (Invitrogen, 13778100). A mock, no-drug control, which consisted of transfecting 1×phosphate-buffered saline, was included. After 72 hours of siNA treatment, the Dual-Glo® Luciferase Assay System (Promega, E2940) was used according to the manufacturer's protocol to quantify firefly and Renilla luciferase activity. All luminescence was measured on an EnVision plate reader (Perkin Elmer). The Renilla:firefly luminescence ratio is calculated for each well. The ratios from siNA-treated wells were then normalized to ratios of the mock-treated wells and percentage inhibition was calculated. CellTiter-Glo© Luminescent Cell Viability Assays were run in parallel using similarly treated COS-7 cells. Assays were performed according to the manufacturer's protocol and luminescence is measured on an EnVision plate reader. The luminescence from siNA-treated wells were then normalized to luminescence of mock-treated wells and percentage viability was calculated.

A subset of siNA candidates were then tested in a dose-response luciferase reporter assay. Dose-response assays were similarly conducted, but instead with serial concentrations of siNAs starting at 10 nM (1:5 dilutions) for a total of nine concentrations tested for each siNA. Dose-response curves were fitted by nonlinear regression with variable slope and EC₅₀ values and maximum percentage inhibition were calculated. No siNAs exhibited significant cytotoxic effects in the COS-7 cells at the concentrations tested.

### Example 59: Identification of siRNA Sequences

In this example, potential siRNA sequences targeting the PNPLA3 I148M variant were identified. The PNPLA3 I148M RefSeq CDS (NCBI Ref. No. NM_025225.3:c.444C>G) (SEQ ID NO: 2067) was used as the starting reference sequence. PNPLA3 I148M RefSeq CDS refers to a human PNPLA3 gene, which is a variant of SEQ ID NO:1 having a single nucleotide substitution at position 444 from a C to G, and encodes a PNPLA3 protein, which is a variant of SEQ ID NO:2 having a single substitution at position 148 of the amino acid sequence which is an I148M substitution. Bioinformatics analysis was used to select target sites and design siRNA molecules with favorable on-target and off-target properties.

A subset of 19-mer and 21-mer siRNA sequences were selected for further investigation. Table 1 includes a list of certain unmodified sense strand and antisense strand 19-mer and 21-mer siRNA sequences.

To improve certain properties of the siRNAs, including, e.g., the potency and/or stability, modified variations of selected sense and antisense sequences were designed and synthesized. The modified sequences included various patterns of siRNA modifications, including, 2′-O-methyl nucleotides, 2′-fluoro nucleotides, 5′ terminal vinyl phosphonate, phosphorothioate internucleoside linkages, and UU overhangs. It will be understood that the nucleotide monomers used in the siRNA sequences are linked by 3′-5′ phosphodiester bonds unless specified otherwise. Table 2 includes a list of certain modified sense and antisense strand 19-mer and 21-mer siRNA sequences.

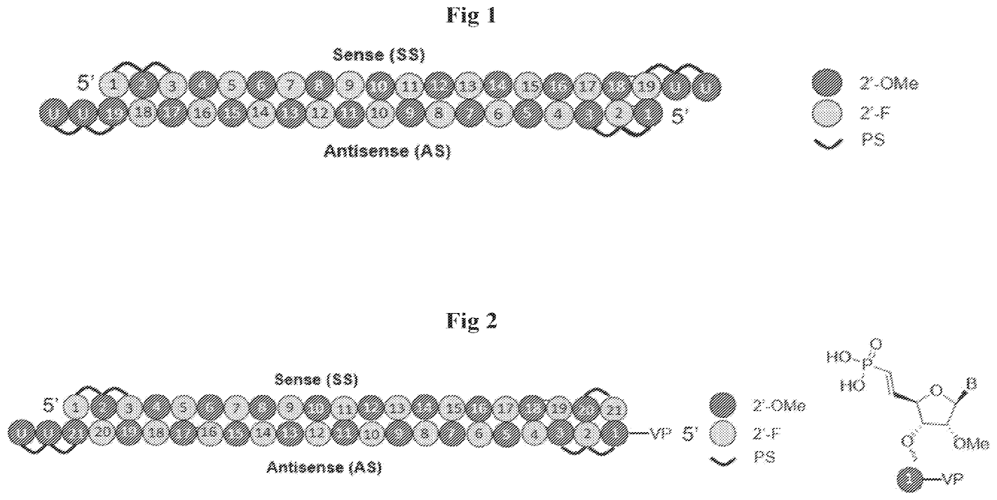

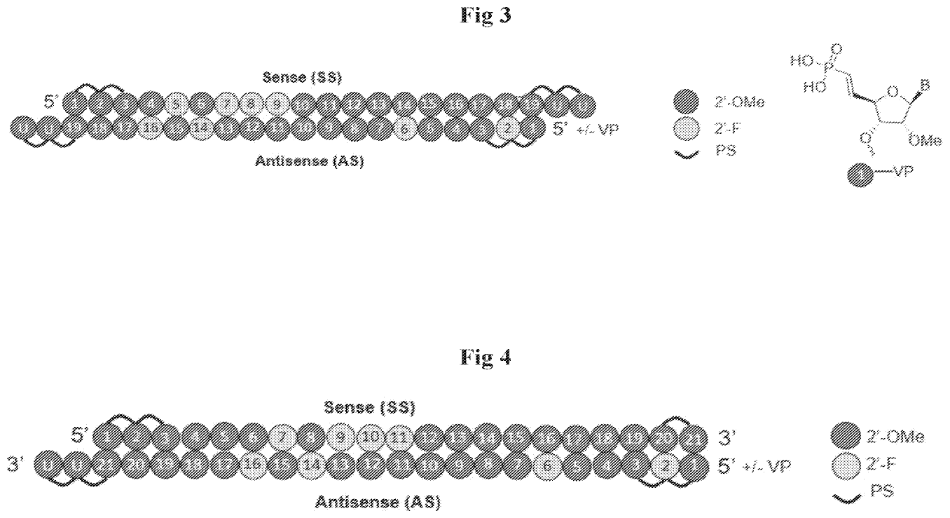

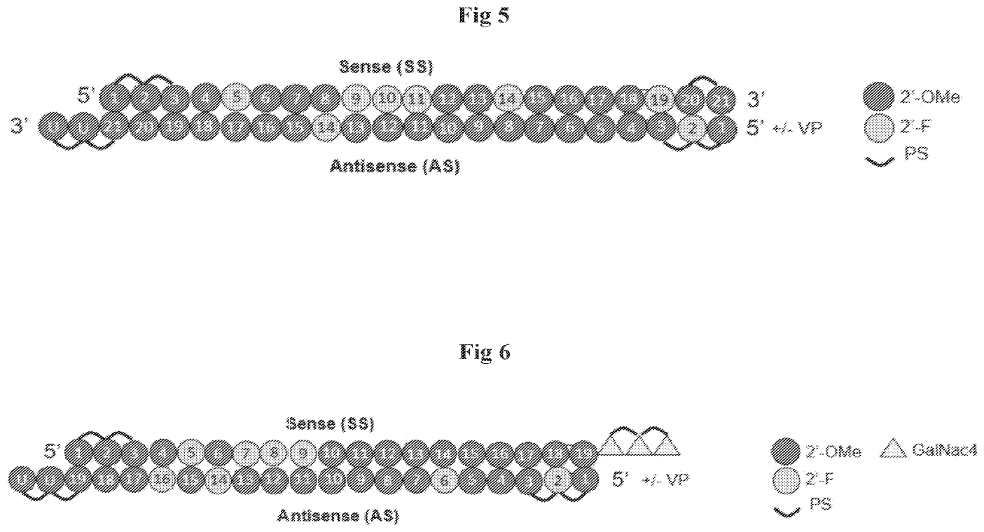

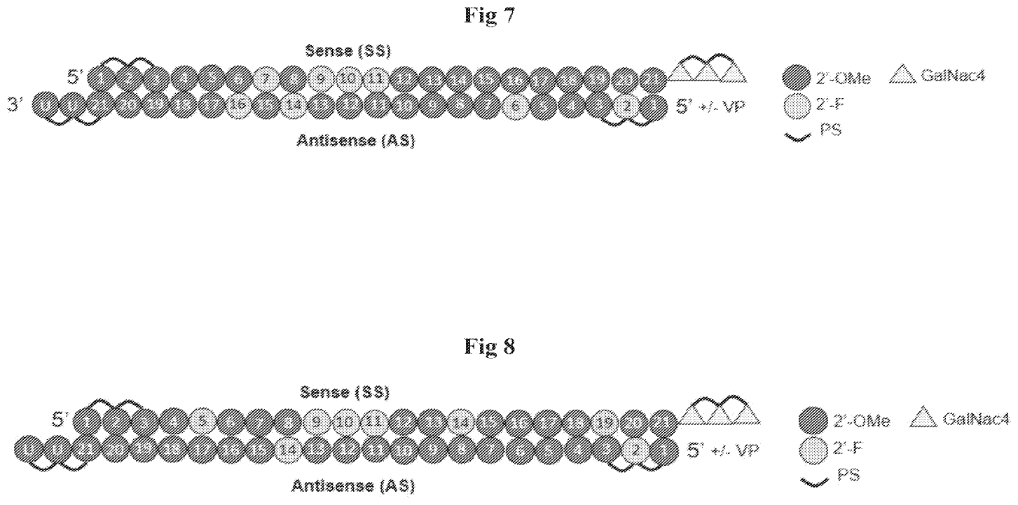

To the extent that a 19-mer or 21-mer includes an unpaired UU overhang at the 3′ end of the sense and/or antisense strand, the overhang is not included in the term “19-mer” or “21-mer”. Specifically, for example, a 21-mer with an unpaired UU overhang at the 3′ end of the antisense strand is called a “21-mer” despite having 23 nucleotides in the antisense strand due to the UU overhang. FIGS. 1 and 3 provide example models of a 19-mer with a UU overhang at the 3′ end of the sense strand and at the 3′ end of the antisense strand. FIGS. 2, 4 and 5 provide example models of a 21-mer with a UU overhang at the 3′ end of the antisense strand.

The present disclosure is not limited to only the specific modifications and/or patterns of modifications disclosed herein. Specifically, for example, one ordinarily skilled in the art would understand that any of the sequences listed in Table 1 could be unmodified, un-conjugated, modified, and/or conjugated, as described herein. For example, any of the siRNA molecules may comprise at least one modified nucleotide, including a vinyl phosphonate or derivative thereof (or an additional vinyl phosphonate) modification at the 3′ end and/or 5′ end of the sense and/or antisense strand, and/or may comprise a GalNAc ligand for in vivo administration as described herein.

TABLE 1
siRNA Sequences

| | | Target | Target | | | |
|---|---|---|---|---|---|---|
| siRNA | | Site Start | Site End | Sense Strand Base | | Antisense Strand Base |
| Duplex | SEQ | Position | Position | Sequence + Chem | SEQ | Sequence + Chem |
| ID NO. | ID | in SEQ ID | in SEQ ID | Modifications | ID | Modifications |
| (Dx) | NO. | NO. 2067 | NO. 2067 | (5′-3′) | NO. | (5′-3′) |
| 1 | 3 | 1 | 19 | AUGUACGACGCAGAGCGCGUU | 453 | CGCGCUCUGCGUCGUACAUUU |
| 2 | 4 | 2 | 20 | UGUACGACGCAGAGCGCGGUU | 454 | CCGCGCUCUGCGUCGUACAUU |
| 3 | 5 | 3 | 21 | GUACGACGCAGAGCGCGGCUU | 455 | GCCGCGCUCUGCGUCGUACUU |
| 4 | 6 | 5 | 23 | ACGACGCAGAGCGCGGCUGUU | 456 | CAGCCGCGCUCUGCGUCGUUU |
| 5 | 7 | 6 | 24 | CGACGCAGAGCGCGGCUGGUU | 457 | CCAGCCGCGCUCUGCGUCGUU |
| 6 | 8 | 7 | 25 | GACGCAGAGCGCGGCUGGAUU | 458 | UCCAGCCGCGCUCUGCGUCUU |
| 7 | 9 | 10 | 28 | GCAGAGCGCGGCUGGAGCUUU | 459 | AGCUCCAGCCGCGCUCUGCUU |
| 8 | 10 | 11 | 29 | CAGAGCGCGGCUGGAGCUUUU | 460 | AAGCUCCAGCCGCGCUCUGUU |
| 9 | 11 | 12 | 30 | AGAGCGCGGCUGGAGCUUGUU | 461 | CAAGCUCCAGCCGCGCUCUUU |
| 10 | 12 | 13 | 31 | GAGCGCGGCUGGAGCUUGUUU | 462 | ACAAGCUCCAGCCGCGCUCUU |
| 11 | 13 | 15 | 33 | GCGCGGCUGGAGCUUGUCCUU | 463 | GGACAAGCUCCAGCCGCGCUU |
| 12 | 14 | 16 | 34 | CGCGGCUGGAGCUUGUCCUUU | 464 | AGGACAAGCUCCAGCCGCGUU |
| 13 | 15 | 17 | 35 | GCGGCUGGAGCUUGUCCUUUU | 465 | AAGGACAAGCUCCAGCCGCUU |
| 14 | 16 | 20 | 38 | GCUGGAGCUUGUCCUUCGCUU | 466 | GCGAAGGACAAGCUCCAGCUU |
| 15 | 17 | 22 | 40 | UGGAGCUUGUCCUUCGCGGUU | 467 | CCGCGAAGGACAAGCUCCAUU |
| 16 | 18 | 23 | 41 | GGAGCUUGUCCUUCGCGGGUU | 468 | CCCGCGAAGGACAAGCUCCUU |
| 17 | 19 | 24 | 42 | GAGCUUGUCCUUCGCGGGCUU | 469 | GCCCGCGAAGGACAAGCUCUU |
| 18 | 20 | 25 | 43 | AGCUUGUCCUUCGCGGGCUUU | 470 | AGCCCGCGAAGGACAAGCUUU |
| 19 | 21 | 30 | 48 | GUCCUUCGCGGGCUGCGGCUU | 471 | GCCGCAGCCCGCGAAGGACUU |
| 20 | 22 | 41 | 59 | GCUGCGGCUUCCUGGGCUUUU | 472 | AAGCCCAGGAAGCCGCAGCUU |
| 21 | 23 | 42 | 60 | CUGCGGCUUCCUGGGCUUCUU | 473 | GAAGCCCAGGAAGCCGCAGUU |
| 22 | 24 | 45 | 63 | CGGCUUCCUGGGCUUCUACUU | 474 | GUAGAAGCCCAGGAAGCCGUU |
| 23 | 25 | 52 | 70 | CUGGGCUUCUACCACGUCGUU | 475 | CGACGUGGUAGAAGCCCAGUU |
| 24 | 26 | 53 | 71 | UGGGCUUCUACCACGUCGGUU | 476 | CCGACGUGGUAGAAGCCCAUU |
| 25 | 27 | 55 | 73 | GGCUUCUACCACGUCGGGGUU | 477 | CCCCGACGUGGUAGAAGCCUU |
| 26 | 28 | 56 | 74 | GCUUCUACCACGUCGGGGCUU | 478 | GCCCCGACGUGGUAGAAGCUU |
| 27 | 29 | 57 | 75 | CUUCUACCACGUCGGGGCGUU | 479 | CGCCCCGACGUGGUAGAAGUU |
| 28 | 30 | 58 | 76 | UUCUACCACGUCGGGGCGAUU | 480 | UCGCCCCGACGUGGUAGAAUU |
| 29 | 31 | 59 | 77 | UCUACCACGUCGGGGCGACUU | 481 | GUCGCCCCGACGUGGUAGAUU |
| 30 | 32 | 60 | 78 | CUACCACGUCGGGGCGACCUU | 482 | GGUCGCCCCGACGUGGUAGUU |
| 31 | 33 | 62 | 80 | ACCACGUCGGGGCGACCCGUU | 483 | CGGGUCGCCCCGACGUGGUUU |
| 32 | 34 | 81 | 99 | CUGCCUGAGCGAGCACGCCUU | 484 | GGCGUGCUCGCUCAGGCAGUU |
| 33 | 35 | 82 | 100 | UGCCUGAGCGAGCACGCCCUU | 485 | GGGCGUGCUCGCUCAGGCAUU |
| 34 | 36 | 84 | 102 | CCUGAGCGAGCACGCCCCGUU | 486 | CGGGGCGUGCUCGCUCAGGUU |
| 35 | 37 | 87 | 105 | GAGCGAGCACGCCCCGCACUU | 487 | GUGCGGGGCGUGCUCGCUCUU |
| 36 | 38 | 88 | 106 | AGCGAGCACGCCCCGCACCUU | 488 | GGUGCGGGGCGUGCUCGCUUU |
| 37 | 39 | 91 | 109 | GAGCACGCCCCGCACCUCCUU | 489 | GGAGGUGCGGGGCGUGCUCUU |
| 38 | 40 | 98 | 116 | CCCCGCACCUCCUCCGCGAUU | 490 | UCGCGGAGGAGGUGCGGGGUU |
| 39 | 41 | 99 | 117 | CCCGCACCUCCUCCGCGACUU | 491 | GUCGCGGAGGAGGUGCGGGUU |
| 40 | 42 | 100 | 118 | CCGCACCUCCUCCGCGACGUU | 492 | CGUCGCGGAGGAGGUGCGGUU |
| 41 | 43 | 101 | 119 | CGCACCUCCUCCGCGACGCUU | 493 | GCGUCGCGGAGGAGGUGCGUU |
| 42 | 44 | 105 | 123 | CCUCCUCCGCGACGCGCGCUU | 494 | GCGCGCGUCGCGGAGGAGGUU |
| 43 | 45 | 106 | 124 | CUCCUCCGCGACGCGCGCAUU | 495 | UGCGCGCGUCGCGGAGGAGUU |
| 44 | 46 | 107 | 125 | UCCUCCGCGACGCGCGCAUUU | 496 | AUGCGCGCGUCGCGGAGGAUU |
| 45 | 47 | 108 | 126 | CCUCCGCGACGCGCGCAUGUU | 497 | CAUGCGCGCGUCGCGGAGGUU |
| 46 | 48 | 109 | 127 | CUCCGCGACGCGCGCAUGUUU | 498 | ACAUGCGCGCGUCGCGGAGUU |
| 47 | 49 | 110 | 128 | UCCGCGACGCGCGCAUGUUUU | 499 | AACAUGCGCGCGUCGCGGAUU |
| 48 | 50 | 111 | 129 | CCGCGACGCGCGCAUGUUGUU | 500 | CAACAUGCGCGCGUCGCGGUU |
| 49 | 51 | 112 | 130 | CGCGACGCGCGCAUGUUGUUU | 501 | ACAACAUGCGCGCGUCGCGUU |
| 50 | 52 | 113 | 131 | GCGACGCGCGCAUGUUGUUUU | 502 | AACAACAUGCGCGCGUCGCUU |
| 51 | 53 | 115 | 133 | GACGCGCGCAUGUUGUUCGUU | 503 | CGAACAACAUGCGCGCGUCUU |
| 52 | 54 | 116 | 134 | ACGCGCGCAUGUUGUUCGGUU | 504 | CCGAACAACAUGCGCGCGUUU |
| 53 | 55 | 117 | 135 | CGCGCGCAUGUUGUUCGGCUU | 505 | GCCGAACAACAUGCGCGCGUU |
| 54 | 56 | 118 | 136 | GCGCGCAUGUUGUUCGGCGUU | 506 | CGCCGAACAACAUGCGCGCUU |
| 55 | 57 | 127 | 145 | UUGUUCGGCGCUUCGGCCGUU | 507 | CGGCCGAAGCGCCGAACAAUU |
| 56 | 58 | 128 | 146 | UGUUCGGCGCUUCGGCCGGUU | 508 | CCGGCCGAAGCGCCGAACAUU |
| 57 | 59 | 139 | 157 | UCGGCCGGGGCGUUGCACUUU | 509 | AGUGCAACGCCCCGGCCGAUU |
| 58 | 60 | 214 | 232 | CUUGUGCGGAAGGCCAGGAUU | 510 | UCCUGGCCUUCCGCACAAGUU |
| 59 | 61 | 216 | 234 | UGUGCGGAAGGCCAGGAGUUU | 511 | ACUCCUGGCCUUCCGCACAUU |
| 60 | 62 | 218 | 236 | UGCGGAAGGCCAGGAGUCGUU | 512 | CGACUCCUGGCCUUCCGCAUU |
| 61 | 63 | 220 | 238 | CGGAAGGCCAGGAGUCGGAUU | 513 | UCCGACUCCUGGCCUUCCGUU |
| 62 | 64 | 223 | 241 | AAGGCCAGGAGUCGGAACAUU | 514 | UGUUCCGACUCCUGGCCUUUU |
| 63 | 65 | 224 | 242 | AGGCCAGGAGUCGGAACAUUU | 515 | AUGUUCCGACUCCUGGCCUUU |
| 64 | 66 | 225 | 243 | GGCCAGGAGUCGGAACAUUUU | 516 | AAUGUUCCGACUCCUGGCCUU |
| 65 | 67 | 228 | 246 | CAGGAGUCGGAACAUUGGCUU | 517 | GCCAAUGUUCCGACUCCUGUU |
| 66 | 68 | 229 | 247 | AGGAGUCGGAACAUUGGCAUU | 518 | UGCCAAUGUUCCGACUCCUUU |
| 67 | 69 | 235 | 253 | CGGAACAUUGGCAUCUUCCUU | 519 | GGAAGAUGCCAAUGUUCCGUU |
| 68 | 70 | 238 | 256 | AACAUUGGCAUCUUCCAUCUU | 520 | GAUGGAAGAUGCCAAUGUUUU |
| 69 | 71 | 239 | 257 | ACAUUGGCAUCUUCCAUCCUU | 521 | GGAUGGAAGAUGCCAAUGUUU |
| 70 | 72 | 259 | 277 | UCCUUCAACUUAAGCAAGUUU | 522 | ACUUGCUUAAGUUGAAGGAUU |
| 71 | 73 | 261 | 279 | CUUCAACUUAAGCAAGUUCUU | 523 | GAACUUGCUUAAGUUGAAGUU |
| 72 | 74 | 302 | 320 | GCCUCCCGGCCAAUGUCCAUU | 524 | UGGACAUUGGCCGGGAGGCUU |
| 73 | 75 | 303 | 321 | CCUCCCGGCCAAUGUCCACUU | 525 | GUGGACAUUGGCCGGGAGGUU |
| 74 | 76 | 311 | 329 | CCAAUGUCCACCAGCUCAUUU | 526 | AUGAGCUGGUGGACAUUGGUU |
| 75 | 77 | 313 | 331 | AAUGUCCACCAGCUCAUCUUU | 527 | AGAUGAGCUGGUGGACAUUUU |
| 76 | 78 | 314 | 332 | AUGUCCACCAGCUCAUCUCUU | 528 | GAGAUGAGCUGGUGGACAUUU |
| 77 | 79 | 316 | 334 | GUCCACCAGCUCAUCUCCGUU | 529 | CGGAGAUGAGCUGGUGGACUU |
| 78 | 80 | 317 | 335 | UCCACCAGCUCAUCUCCGGUU | 530 | CCGGAGAUGAGCUGGUGGAUU |
| 79 | 81 | 321 | 339 | CCAGCUCAUCUCCGGCAAAUU | 531 | UUUGCCGGAGAUGAGCUGGUU |
| 80 | 82 | 343 | 361 | GGCAUCUCUCUUACCAGAGUU | 532 | CUCUGGUAAGAGAGAUGCCUU |
| 81 | 83 | 351 | 369 | UCUUACCAGAGUGUCUGAUUU | 533 | AUCAGACACUCUGGUAAGAUU |
| 82 | 84 | 352 | 370 | CUUACCAGAGUGUCUGAUGUU | 534 | CAUCAGACACUCUGGUAAGUU |
| 83 | 85 | 353 | 371 | UUACCAGAGUGUCUGAUGGUU | 535 | CCAUCAGACACUCUGGUAAUU |
| 84 | 86 | 354 | 372 | UACCAGAGUGUCUGAUGGGUU | 536 | CCCAUCAGACACUCUGGUAUU |
| 85 | 87 | 359 | 377 | GAGUGUCUGAUGGGGAAAAUU | 537 | UUUUCCCCAUCAGACACUCUU |
| 86 | 88 | 361 | 379 | GUGUCUGAUGGGGAAAACGUU | 538 | CGUUUUCCCCAUCAGACACUU |
| 87 | 89 | 362 | 380 | UGUCUGAUGGGGAAAACGUUU | 539 | ACGUUUUCCCCAUCAGACAUU |
| 88 | 90 | 363 | 381 | GUCUGAUGGGGAAAACGUUUU | 540 | AACGUUUUCCCCAUCAGACUU |
| 89 | 91 | 364 | 382 | UCUGAUGGGGAAAACGUUCUU | 541 | GAACGUUUUCCCCAUCAGAUU |
| 90 | 92 | 365 | 383 | CUGAUGGGGAAAACGUUCUUU | 542 | AGAACGUUUUCCCCAUCAGUU |
| 91 | 93 | 366 | 384 | UGAUGGGGAAAACGUUCUGUU | 543 | CAGAACGUUUUCCCCAUCAUU |
| 92 | 94 | 367 | 385 | GAUGGGGAAAACGUUCUGGUU | 544 | CCAGAACGUUUUCCCCAUCUU |
| 93 | 95 | 369 | 387 | UGGGGAAAACGUUCUGGUGUU | 545 | CACCAGAACGUUUUCCCCAUU |
| 94 | 96 | 371 | 389 | GGGAAAACGUUCUGGUGUCUU | 546 | GACACCAGAACGUUUUCCCUU |
| 95 | 97 | 372 | 390 | GGAAAACGUUCUGGUGUCUUU | 547 | AGACACCAGAACGUUUUCCUU |
| 96 | 98 | 375 | 393 | AAACGUUCUGGUGUCUGACUU | 548 | GUCAGACACCAGAACGUUUUU |
| 97 | 99 | 376 | 394 | AACGUUCUGGUGUCUGACUUU | 549 | AGUCAGACACCAGAACGUUUU |
| 98 | 100 | 377 | 395 | ACGUUCUGGUGUCUGACUUUU | 550 | AAGUCAGACACCAGAACGUUU |
| 99 | 101 | 378 | 396 | CGUUCUGGUGUCUGACUUUUU | 551 | AAAGUCAGACACCAGAACGUU |
| 100 | 102 | 379 | 397 | GUUCUGGUGUCUGACUUUCUU | 552 | GAAAGUCAGACACCAGAACUU |
| 101 | 103 | 380 | 398 | UUCUGGUGUCUGACUUUCGUU | 553 | CGAAAGUCAGACACCAGAAUU |
| 102 | 104 | 382 | 400 | CUGGUGUCUGACUUUCGGUUU | 554 | ACCGAAAGUCAGACACCAGUU |
| 103 | 105 | 383 | 401 | UGGUGUCUGACUUUCGGUCUU | 555 | GACCGAAAGUCAGACACCAUU |
| 104 | 106 | 384 | 402 | GGUGUCUGACUUUCGGUCCUU | 556 | GGACCGAAAGUCAGACACCUU |
| 105 | 107 | 385 | 403 | GUGUCUGACUUUCGGUCCAUU | 557 | UGGACCGAAAGUCAGACACUU |
| 106 | 108 | 386 | 404 | UGUCUGACUUUCGGUCCAAUU | 558 | UUGGACCGAAAGUCAGACAUU |
| 107 | 109 | 387 | 405 | GUCUGACUUUCGGUCCAAAUU | 559 | UUUGGACCGAAAGUCAGACUU |
| 108 | 110 | 398 | 416 | GGUCCAAAGACGAAGUCGUUU | 560 | ACGACUUCGUCUUUGGACCUU |
| 109 | 111 | 399 | 417 | GUCCAAAGACGAAGUCGUGUU | 561 | CACGACUUCGUCUUUGGACUU |
| 110 | 112 | 400 | 418 | UCCAAAGACGAAGUCGUGGUU | 562 | CCACGACUUCGUCUUUGGAUU |
| 111 | 113 | 401 | 419 | CCAAAGACGAAGUCGUGGAUU | 563 | UCCACGACUUCGUCUUUGGUU |
| 112 | 114 | 402 | 420 | CAAAGACGAAGUCGUGGAUUU | 564 | AUCCACGACUUCGUCUUUGUU |
| 113 | 115 | 404 | 422 | AAGACGAAGUCGUGGAUGCUU | 565 | GCAUCCACGACUUCGUCUUUU |
| 114 | 116 | 405 | 423 | AGACGAAGUCGUGGAUGCCUU | 566 | GGCAUCCACGACUUCGUCUUU |
| 115 | 117 | 406 | 424 | GACGAAGUCGUGGAUGCCUUU | 567 | AGGCAUCCACGACUUCGUCUU |
| 116 | 118 | 407 | 425 | ACGAAGUCGUGGAUGCCUUUU | 568 | AAGGCAUCCACGACUUCGUUU |
| 117 | 119 | 409 | 427 | GAAGUCGUGGAUGCCUUGGUU | 569 | CCAAGGCAUCCACGACUUCUU |
| 118 | 120 | 410 | 428 | AAGUCGUGGAUGCCUUGGUUU | 570 | ACCAAGGCAUCCACGACUUUU |
| 119 | 121 | 412 | 430 | GUCGUGGAUGCCUUGGUAUUU | 571 | AUACCAAGGCAUCCACGACUU |
| 120 | 122 | 413 | 431 | UCGUGGAUGCCUUGGUAUGUU | 572 | CAUACCAAGGCAUCCACGAUU |
| 121 | 123 | 415 | 433 | GUGGAUGCCUUGGUAUGUUUU | 573 | AACAUACCAAGGCAUCCACUU |
| 122 | 124 | 416 | 434 | UGGAUGCCUUGGUAUGUUCUU | 574 | GAACAUACCAAGGCAUCCAUU |
| 123 | 125 | 417 | 435 | GGAUGCCUUGGUAUGUUCCUU | 575 | GGAACAUACCAAGGCAUCCUU |
| 124 | 126 | 419 | 437 | AUGCCUUGGUAUGUUCCUGUU | 576 | CAGGAACAUACCAAGGCAUUU |
| 125 | 127 | 421 | 439 | GCCUUGGUAUGUUCCUGCUUU | 577 | AGCAGGAACAUACCAAGGCUU |
| 126 | 128 | 429 | 447 | AUGUUCCUGCUUCAUGCCCUU | 578 | GGGCAUGAAGCAGGAACAUUU |
| 127 | 129 | 432 | 450 | UUCCUGCUUCAUGCCCUUCUU | 579 | GAAGGGCAUGAAGCAGGAAUU |
| 128 | 130 | 433 | 451 | UCCUGCUUCAUGCCCUUCUUU | 580 | AGAAGGGCAUGAAGCAGGAUU |
| 129 | 131 | 456 | 474 | UGGCCUUAUCCCUCCUUCCUU | 581 | GGAAGGAGGGAUAAGGCCAUU |
| 130 | 132 | 461 | 479 | UUAUCCCUCCUUCCUUCAGUU | 582 | CUGAAGGAAGGAGGGAUAAUU |
| 131 | 133 | 462 | 480 | UAUCCCUCCUUCCUUCAGAUU | 583 | UCUGAAGGAAGGAGGGAUAUU |
| 132 | 134 | 466 | 484 | CCUCCUUCCUUCAGAGGCGUU | 584 | CGCCUCUGAAGGAAGGAGGUU |
| 133 | 135 | 467 | 485 | CUCCUUCCUUCAGAGGCGUUU | 585 | ACGCCUCUGAAGGAAGGAGUU |
| 134 | 136 | 469 | 487 | CCUUCCUUCAGAGGCGUGCUU | 586 | GCACGCCUCUGAAGGAAGGUU |
| 135 | 137 | 470 | 488 | CUUCCUUCAGAGGCGUGCGUU | 587 | CGCACGCCUCUGAAGGAAGUU |
| 136 | 138 | 471 | 489 | UUCCUUCAGAGGCGUGCGAUU | 588 | UCGCACGCCUCUGAAGGAAUU |
| 137 | 139 | 472 | 490 | UCCUUCAGAGGCGUGCGAUUU | 589 | AUCGCACGCCUCUGAAGGAUU |
| 138 | 140 | 473 | 491 | CCUUCAGAGGCGUGCGAUAUU | 590 | UAUCGCACGCCUCUGAAGGUU |
| 139 | 141 | 489 | 507 | AUAUGUGGAUGGAGGAGUGUU | 591 | CACUCCUCCAUCCACAUAUUU |
| 140 | 142 | 494 | 512 | UGGAUGGAGGAGUGAGUGAUU | 592 | UCACUCACUCCUCCAUCCAUU |
| 141 | 143 | 497 | 515 | AUGGAGGAGUGAGUGACAAUU | 593 | UUGUCACUCACUCCUCCAUUU |
| 142 | 144 | 501 | 519 | AGGAGUGAGUGACAACGUAUU | 594 | UACGUUGUCACUCACUCCUUU |
| 143 | 145 | 502 | 520 | GGAGUGAGUGACAACGUACUU | 595 | GUACGUUGUCACUCACUCCUU |
| 144 | 146 | 504 | 522 | AGUGAGUGACAACGUACCCUU | 596 | GGGUACGUUGUCACUCACUUU |
| 145 | 147 | 505 | 523 | GUGAGUGACAACGUACCCUUU | 597 | AGGGUACGUUGUCACUCACUU |
| 146 | 148 | 507 | 525 | GAGUGACAACGUACCCUUCUU | 598 | GAAGGGUACGUUGUCACUCUU |
| 147 | 149 | 530 | 548 | AUGCCAAAACAACCAUCACUU | 599 | GUGAUGGUUGUUUUGGCAUUU |
| 148 | 150 | 531 | 549 | UGCCAAAACAACCAUCACCUU | 600 | GGUGAUGGUUGUUUUGGCAUU |
| 149 | 151 | 532 | 550 | GCCAAAACAACCAUCACCGUU | 601 | CGGUGAUGGUUGUUUUGGCUU |
| 150 | 152 | 535 | 553 | AAAACAACCAUCACCGUGUUU | 602 | ACACGGUGAUGGUUGUUUUUU |
| 151 | 153 | 536 | 554 | AAACAACCAUCACCGUGUCUU | 603 | GACACGGUGAUGGUUGUUUUU |
| 152 | 154 | 538 | 556 | ACAACCAUCACCGUGUCCCUU | 604 | GGGACACGGUGAUGGUUGUUU |
| 153 | 155 | 539 | 557 | CAACCAUCACCGUGUCCCCUU | 605 | GGGGACACGGUGAUGGUUGUU |
| 154 | 156 | 540 | 558 | AACCAUCACCGUGUCCCCCUU | 606 | GGGGGACACGGUGAUGGUUUU |
| 155 | 157 | 546 | 564 | CACCGUGUCCCCCUUCUAUUU | 607 | AUAGAAGGGGGACACGGUGUU |
| 156 | 158 | 547 | 565 | ACCGUGUCCCCCUUCUAUGUU | 608 | CAUAGAAGGGGGACACGGUUU |
| 157 | 159 | 550 | 568 | GUGUCCCCCUUCUAUGGGGUU | 609 | CCCCAUAGAAGGGGGACACUU |
| 158 | 160 | 551 | 569 | UGUCCCCCUUCUAUGGGGAUU | 610 | UCCCCAUAGAAGGGGGACAUU |
| 159 | 161 | 552 | 570 | GUCCCCCUUCUAUGGGGAGUU | 611 | CUCCCCAUAGAAGGGGGACUU |
| 160 | 162 | 553 | 571 | UCCCCCUUCUAUGGGGAGUUU | 612 | ACUCCCCAUAGAAGGGGGAUU |
| 161 | 163 | 556 | 574 | CCCUUCUAUGGGGAGUACGUU | 613 | CGUACUCCCCAUAGAAGGGUU |
| 162 | 164 | 557 | 575 | CCUUCUAUGGGGAGUACGAUU | 614 | UCGUACUCCCCAUAGAAGGUU |
| 163 | 165 | 559 | 577 | UUCUAUGGGGAGUACGACAUU | 615 | UGUCGUACUCCCCAUAGAAUU |
| 164 | 166 | 560 | 578 | UCUAUGGGGAGUACGACAUUU | 616 | AUGUCGUACUCCCCAUAGAUU |
| 165 | 167 | 561 | 579 | CUAUGGGGAGUACGACAUCUU | 617 | GAUGUCGUACUCCCCAUAGUU |
| 166 | 168 | 562 | 580 | UAUGGGGAGUACGACAUCUUU | 618 | AGAUGUCGUACUCCCCAUAUU |
| 167 | 169 | 571 | 589 | UACGACAUCUGCCCUAAAGUU | 619 | CUUUAGGGCAGAUGUCGUAUU |
| 168 | 170 | 572 | 590 | ACGACAUCUGCCCUAAAGUUU | 620 | ACUUUAGGGCAGAUGUCGUUU |
| 169 | 171 | 573 | 591 | CGACAUCUGCCCUAAAGUCUU | 621 | GACUUUAGGGCAGAUGUCGUU |
| 170 | 172 | 574 | 592 | GACAUCUGCCCUAAAGUCAUU | 622 | UGACUUUAGGGCAGAUGUCUU |
| 171 | 173 | 575 | 593 | ACAUCUGCCCUAAAGUCAAUU | 623 | UUGACUUUAGGGCAGAUGUUU |
| 172 | 174 | 576 | 594 | CAUCUGCCCUAAAGUCAAGUU | 624 | CUUGACUUUAGGGCAGAUGUU |
| 173 | 175 | 577 | 595 | AUCUGCCCUAAAGUCAAGUUU | 625 | ACUUGACUUUAGGGCAGAUUU |
| 174 | 176 | 582 | 600 | CCCUAAAGUCAAGUCCACGUU | 626 | CGUGGACUUGACUUUAGGGUU |
| 175 | 177 | 583 | 601 | CCUAAAGUCAAGUCCACGAUU | 627 | UCGUGGACUUGACUUUAGGUU |
| 176 | 178 | 585 | 603 | UAAAGUCAAGUCCACGAACUU | 628 | GUUCGUGGACUUGACUUUAUU |
| 177 | 179 | 586 | 604 | AAAGUCAAGUCCACGAACUUU | 629 | AGUUCGUGGACUUGACUUUUU |
| 178 | 180 | 587 | 605 | AAGUCAAGUCCACGAACUUUU | 630 | AAGUUCGUGGACUUGACUUUU |
| 179 | 181 | 588 | 606 | AGUCAAGUCCACGAACUUUUU | 631 | AAAGUUCGUGGACUUGACUUU |
| 180 | 182 | 589 | 607 | GUCAAGUCCACGAACUUUCUU | 632 | GAAAGUUCGUGGACUUGACUU |
| 181 | 183 | 590 | 608 | UCAAGUCCACGAACUUUCUUU | 633 | AGAAAGUUCGUGGACUUGAUU |
| 182 | 184 | 591 | 609 | CAAGUCCACGAACUUUCUUUU | 634 | AAGAAAGUUCGUGGACUUGUU |
| 183 | 185 | 592 | 610 | AAGUCCACGAACUUUCUUCUU | 635 | GAAGAAAGUUCGUGGACUUUU |
| 184 | 186 | 593 | 611 | AGUCCACGAACUUUCUUCAUU | 636 | UGAAGAAAGUUCGUGGACUUU |
| 185 | 187 | 594 | 612 | GUCCACGAACUUUCUUCAUUU | 637 | AUGAAGAAAGUUCGUGGACUU |
| 186 | 188 | 595 | 613 | UCCACGAACUUUCUUCAUGUU | 638 | CAUGAAGAAAGUUCGUGGAUU |
| 187 | 189 | 597 | 615 | CACGAACUUUCUUCAUGUGUU | 639 | CACAUGAAGAAAGUUCGUGUU |
| 188 | 190 | 620 | 638 | UCACCAAGCUCAGUCUACGUU | 640 | CGUAGACUGAGCUUGGUGAUU |
| 189 | 191 | 621 | 639 | CACCAAGCUCAGUCUACGCUU | 641 | GCGUAGACUGAGCUUGGUGUU |
| 190 | 192 | 622 | 640 | ACCAAGCUCAGUCUACGCCUU | 642 | GGCGUAGACUGAGCUUGGUUU |
| 191 | 193 | 623 | 641 | CCAAGCUCAGUCUACGCCUUU | 643 | AGGCGUAGACUGAGCUUGGUU |
| 192 | 194 | 624 | 642 | CAAGCUCAGUCUACGCCUCUU | 644 | GAGGCGUAGACUGAGCUUGUU |
| 193 | 195 | 626 | 644 | AGCUCAGUCUACGCCUCUGUU | 645 | CAGAGGCGUAGACUGAGCUUU |
| 194 | 196 | 630 | 648 | CAGUCUACGCCUCUGCACAUU | 646 | UGUGCAGAGGCGUAGACUGUU |
| 195 | 197 | 631 | 649 | AGUCUACGCCUCUGCACAGUU | 647 | CUGUGCAGAGGCGUAGACUUU |
| 196 | 198 | 636 | 654 | ACGCCUCUGCACAGGGAACUU | 648 | GUUCCCUGUGCAGAGGCGUUU |
| 197 | 199 | 642 | 660 | CUGCACAGGGAACCUCUACUU | 649 | GUAGAGGUUCCCUGUGCAGUU |
| 198 | 200 | 643 | 661 | UGCACAGGGAACCUCUACCUU | 650 | GGUAGAGGUUCCCUGUGCAUU |
| 199 | 201 | 646 | 664 | ACAGGGAACCUCUACCUUCUU | 651 | GAAGGUAGAGGUUCCCUGUUU |
| 200 | 202 | 691 | 709 | CUCAAGGUGCUGGGAGAGAUU | 652 | UCUCUCCCAGCACCUUGAGUU |
| 201 | 203 | 694 | 712 | AAGGUGCUGGGAGAGAUAUUU | 653 | AUAUCUCUCCCAGCACCUUUU |
| 202 | 204 | 695 | 713 | AGGUGCUGGGAGAGAUAUGUU | 654 | CAUAUCUCUCCCAGCACCUUU |
| 203 | 205 | 696 | 714 | GGUGCUGGGAGAGAUAUGCUU | 655 | GCAUAUCUCUCCCAGCACCUU |
| 204 | 206 | 697 | 715 | GUGCUGGGAGAGAUAUGCCUU | 656 | GGCAUAUCUCUCCCAGCACUU |
| 205 | 207 | 698 | 716 | UGCUGGGAGAGAUAUGCCUUU | 657 | AGGCAUAUCUCUCCCAGCAUU |
| 206 | 208 | 703 | 721 | GGAGAGAUAUGCCUUCGAGUU | 658 | CUCGAAGGCAUAUCUCUCCUU |
| 207 | 209 | 735 | 753 | AUUCAGGUUCUUGGAAGAGUU | 659 | CUCUUCCAAGAACCUGAAUUU |
| 208 | 210 | 737 | 755 | UCAGGUUCUUGGAAGAGAAUU | 660 | UUCUCUUCCAAGAACCUGAUU |
| 209 | 211 | 743 | 761 | UCUUGGAAGAGAAGGGCAUUU | 661 | AUGCCCUUCUCUUCCAAGAUU |
| 210 | 212 | 744 | 762 | CUUGGAAGAGAAGGGCAUCUU | 662 | GAUGCCCUUCUCUUCCAAGUU |
| 211 | 213 | 745 | 763 | UUGGAAGAGAAGGGCAUCUUU | 663 | AGAUGCCCUUCUCUUCCAAUU |
| 212 | 214 | 747 | 765 | GGAAGAGAAGGGCAUCUGCUU | 664 | GCAGAUGCCCUUCUCUUCCUU |
| 213 | 215 | 748 | 766 | GAAGAGAAGGGCAUCUGCAUU | 665 | UGCAGAUGCCCUUCUCUUCUU |
| 214 | 216 | 749 | 767 | AAGAGAAGGGCAUCUGCAAUU | 666 | UUGCAGAUGCCCUUCUCUUUU |
| 215 | 217 | 752 | 770 | AGAAGGGCAUCUGCAACAGUU | 667 | CUGUUGCAGAUGCCCUUCUUU |
| 216 | 218 | 755 | 773 | AGGGCAUCUGCAACAGGCCUU | 668 | GGCCUGUUGCAGAUGCCCUUU |
| 217 | 219 | 758 | 776 | GCAUCUGCAACAGGCCCCAUU | 669 | UGGGGCCUGUUGCAGAUGCUU |
| 218 | 220 | 977 | 995 | CAGCACUGAGUGAAGAAAUUU | 670 | AUUUCUUCACUCAGUGCUGUU |
| 219 | 221 | 1026 | 1044 | UUGCAACUUGCUACCCAUUUU | 671 | AAUGGGUAGCAAGUUGCAAUU |
| 220 | 222 | 1035 | 1053 | GCUACCCAUUAGGAUAAUGUU | 672 | CAUUAUCCUAAUGGGUAGCUU |
| 221 | 223 | 1038 | 1056 | ACCCAUUAGGAUAAUGUCUUU | 673 | AGACAUUAUCCUAAUGGGUUU |
| 222 | 224 | 1039 | 1057 | CCCAUUAGGAUAAUGUCUUUU | 674 | AAGACAUUAUCCUAAUGGGUU |
| 223 | 225 | 1041 | 1059 | CAUUAGGAUAAUGUCUUAUUU | 675 | AUAAGACAUUAUCCUAAUGUU |
| 224 | 226 | 1064 | 1082 | UGCUGCCCUGUACCCUGCCUU | 676 | GGCAGGGUACAGGGCAGCAUU |
| 225 | 227 | 1068 | 1086 | GCCCUGUACCCUGCCUGUGUU | 677 | CACAGGCAGGGUACAGGGCUU |
| 226 | 228 | 1077 | 1095 | CCUGCCUGUGGAAUCUGCCUU | 678 | GGCAGAUUCCACAGGCAGGUU |
| 227 | 229 | 1080 | 1098 | GCCUGUGGAAUCUGCCAUUUU | 679 | AAUGGCAGAUUCCACAGGCUU |
| 228 | 230 | 1111 | 1129 | AGACUGGUGACAUGGCUUCUU | 680 | GAAGCCAUGUCACCAGUCUUU |
| 229 | 231 | 1196 | 1214 | UGUGUCUGCUCCCCGCCUCUU | 681 | GAGGCGGGGAGCAGACACAUU |
| 230 | 232 | 1198 | 1216 | UGUCUGCUCCCCGCCUCCAUU | 682 | UGGAGGCGGGGAGCAGACAUU |
| 231 | 233 | 1 | 21 | AUGUACGACGCAGAGCGCGGC | 683 | GCCGCGCUCUGCGUCGUACAUUU |
| 232 | 234 | 3 | 23 | GUACGACGCAGAGCGCGGCUG | 684 | CAGCCGCGCUCUGCGUCGUACUU |
| 233 | 235 | 4 | 24 | UACGACGCAGAGCGCGGCUGG | 685 | CCAGCCGCGCUCUGCGUCGUAUU |
| 234 | 236 | 5 | 25 | ACGACGCAGAGCGCGGCUGGA | 686 | UCCAGCCGCGCUCUGCGUCGUUU |
| 235 | 237 | 8 | 28 | ACGCAGAGCGCGGCUGGAGCU | 687 | AGCUCCAGCCGCGCUCUGCGUUU |
| 236 | 238 | 9 | 29 | CGCAGAGCGCGGCUGGAGCUU | 688 | AAGCUCCAGCCGCGCUCUGCGUU |
| 237 | 239 | 10 | 30 | GCAGAGCGCGGCUGGAGCUUG | 689 | CAAGCUCCAGCCGCGCUCUGCUU |
| 238 | 240 | 11 | 31 | CAGAGCGCGGCUGGAGCUUGU | 690 | ACAAGCUCCAGCCGCGCUCUGUU |
| 239 | 241 | 12 | 32 | AGAGCGCGGCUGGAGCUUGUC | 691 | GACAAGCUCCAGCCGCGCUCUUU |
| 240 | 242 | 13 | 33 | GAGCGCGGCUGGAGCUUGUCC | 692 | GGACAAGCUCCAGCCGCGCUCUU |
| 241 | 243 | 15 | 35 | GCGCGGCUGGAGCUUGUCCUU | 693 | AAGGACAAGCUCCAGCCGCGCUU |
| 242 | 244 | 17 | 37 | GCGGCUGGAGCUUGUCCUUCG | 694 | CGAAGGACAAGCUCCAGCCGCUU |
| 243 | 245 | 20 | 40 | GCUGGAGCUUGUCCUUCGCGG | 695 | CCGCGAAGGACAAGCUCCAGCUU |
| 244 | 246 | 22 | 42 | UGGAGCUUGUCCUUCGCGGGC | 696 | GCCCGCGAAGGACAAGCUCCAUU |
| 245 | 247 | 23 | 43 | GGAGCUUGUCCUUCGCGGGCU | 697 | AGCCCGCGAAGGACAAGCUCCUU |
| 246 | 248 | 24 | 44 | GAGCUUGUCCUUCGCGGGCUG | 698 | CAGCCCGCGAAGGACAAGCUCUU |
| 247 | 249 | 25 | 45 | AGCUUGUCCUUCGCGGGCUGC | 699 | GCAGCCCGCGAAGGACAAGCUUU |
| 248 | 250 | 28 | 48 | UUGUCCUUCGCGGGCUGCGGC | 700 | GCCGCAGCCCGCGAAGGACAAUU |
| 249 | 251 | 29 | 49 | UGUCCUUCGCGGGCUGCGGCU | 701 | AGCCGCAGCCCGCGAAGGACAUU |
| 250 | 252 | 30 | 50 | GUCCUUCGCGGGCUGCGGCUU | 702 | AAGCCGCAGCCCGCGAAGGACUU |
| 251 | 253 | 40 | 60 | GGCUGCGGCUUCCUGGGCUUC | 703 | GAAGCCCAGGAAGCCGCAGCCUU |
| 252 | 254 | 41 | 61 | GCUGCGGCUUCCUGGGCUUCU | 704 | AGAAGCCCAGGAAGCCGCAGCUU |
| 253 | 255 | 42 | 62 | CUGCGGCUUCCUGGGCUUCUA | 705 | UAGAAGCCCAGGAAGCCGCAGUU |
| 254 | 256 | 43 | 63 | UGCGGCUUCCUGGGCUUCUAC | 706 | GUAGAAGCCCAGGAAGCCGCAUU |
| 255 | 257 | 44 | 64 | GCGGCUUCCUGGGCUUCUACC | 707 | GGUAGAAGCCCAGGAAGCCGCUU |
| 256 | 258 | 48 | 68 | CUUCCUGGGCUUCUACCACGU | 708 | ACGUGGUAGAAGCCCAGGAAGUU |
| 257 | 259 | 52 | 72 | CUGGGCUUCUACCACGUCGGG | 709 | CCCGACGUGGUAGAAGCCCAGUU |
| 258 | 260 | 53 | 73 | UGGGCUUCUACCACGUCGGGG | 710 | CCCCGACGUGGUAGAAGCCCAUU |
| 259 | 261 | 55 | 75 | GGCUUCUACCACGUCGGGGCG | 711 | CGCCCCGACGUGGUAGAAGCCUU |
| 260 | 262 | 56 | 76 | GCUUCUACCACGUCGGGGCGA | 712 | UCGCCCCGACGUGGUAGAAGCUU |
| 261 | 263 | 57 | 77 | CUUCUACCACGUCGGGGCGAC | 713 | GUCGCCCCGACGUGGUAGAAGUU |
| 262 | 264 | 58 | 78 | UUCUACCACGUCGGGGCGACC | 714 | GGUCGCCCCGACGUGGUAGAAUU |
| 263 | 265 | 60 | 80 | CUACCACGUCGGGGCGACCCG | 715 | CGGGUCGCCCCGACGUGGUAGUU |
| 264 | 266 | 82 | 102 | UGCCUGAGCGAGCACGCCCCG | 716 | CGGGGCGUGCUCGCUCAGGCAUU |
| 265 | 267 | 83 | 103 | GCCUGAGCGAGCACGCCCCGC | 717 | GCGGGGCGUGCUCGCUCAGGCUU |
| 266 | 268 | 86 | 106 | UGAGCGAGCACGCCCCGCACC | 718 | GGUGCGGGGCGUGCUCGCUCAUU |
| 267 | 269 | 88 | 108 | AGCGAGCACGCCCCGCACCUC | 719 | GAGGUGCGGGGCGUGCUCGCUUU |
| 268 | 270 | 89 | 109 | GCGAGCACGCCCCGCACCUCC | 720 | GGAGGUGCGGGGCGUGCUCGCUU |
| 269 | 271 | 91 | 111 | GAGCACGCCCCGCACCUCCUC | 721 | GAGGAGGUGCGGGGCGUGCUCUU |
| 270 | 272 | 96 | 116 | CGCCCCGCACCUCCUCCGCGA | 722 | UCGCGGAGGAGGUGCGGGGCGUU |
| 271 | 273 | 97 | 117 | GCCCCGCACCUCCUCCGCGAC | 723 | GUCGCGGAGGAGGUGCGGGGCUU |
| 272 | 274 | 98 | 118 | CCCCGCACCUCCUCCGCGACG | 724 | CGUCGCGGAGGAGGUGCGGGGUU |
| 273 | 275 | 99 | 119 | CCCGCACCUCCUCCGCGACGC | 725 | GCGUCGCGGAGGAGGUGCGGGUU |
| 274 | 276 | 100 | 120 | CCGCACCUCCUCCGCGACGCG | 726 | CGCGUCGCGGAGGAGGUGCGGUU |
| 275 | 277 | 101 | 121 | CGCACCUCCUCCGCGACGCGC | 727 | GCGCGUCGCGGAGGAGGUGCGUU |
| 276 | 278 | 103 | 123 | CACCUCCUCCGCGACGCGCGC | 728 | GCGCGCGUCGCGGAGGAGGUGUU |
| 277 | 279 | 105 | 125 | CCUCCUCCGCGACGCGCGCAU | 729 | AUGCGCGCGUCGCGGAGGAGGUU |
| 278 | 280 | 106 | 126 | CUCCUCCGCGACGCGCGCAUG | 730 | CAUGCGCGCGUCGCGGAGGAGUU |
| 279 | 281 | 107 | 127 | UCCUCCGCGACGCGCGCAUGU | 731 | ACAUGCGCGCGUCGCGGAGGAUU |
| 280 | 282 | 108 | 128 | CCUCCGCGACGCGCGCAUGUU | 732 | AACAUGCGCGCGUCGCGGAGGUU |
| 281 | 283 | 109 | 129 | CUCCGCGACGCGCGCAUGUUG | 733 | CAACAUGCGCGCGUCGCGGAGUU |
| 282 | 284 | 110 | 130 | UCCGCGACGCGCGCAUGUUGU | 734 | ACAACAUGCGCGCGUCGCGGAUU |
| 283 | 285 | 111 | 131 | CCGCGACGCGCGCAUGUUGUU | 735 | AACAACAUGCGCGCGUCGCGGUU |
| 284 | 286 | 113 | 133 | GCGACGCGCGCAUGUUGUUCG | 736 | CGAACAACAUGCGCGCGUCGCUU |
| 285 | 287 | 114 | 134 | CGACGCGCGCAUGUUGUUCGG | 737 | CCGAACAACAUGCGCGCGUCGUU |
| 286 | 288 | 115 | 135 | GACGCGCGCAUGUUGUUCGGC | 738 | GCCGAACAACAUGCGCGCGUCUU |
| 287 | 289 | 213 | 233 | UCUUGUGCGGAAGGCCAGGAG | 739 | CUCCUGGCCUUCCGCACAAGAUU |
| 288 | 290 | 214 | 234 | CUUGUGCGGAAGGCCAGGAGU | 740 | ACUCCUGGCCUUCCGCACAAGUU |
| 289 | 291 | 216 | 236 | UGUGCGGAAGGCCAGGAGUCG | 741 | CGACUCCUGGCCUUCCGCACAUU |
| 290 | 292 | 218 | 238 | UGCGGAAGGCCAGGAGUCGGA | 742 | UCCGACUCCUGGCCUUCCGCAUU |
| 291 | 293 | 220 | 240 | CGGAAGGCCAGGAGUCGGAAC | 743 | GUUCCGACUCCUGGCCUUCCGUU |
| 292 | 294 | 223 | 243 | AAGGCCAGGAGUCGGAACAUU | 744 | AAUGUUCCGACUCCUGGCCUUUU |
| 293 | 295 | 224 | 244 | AGGCCAGGAGUCGGAACAUUG | 745 | CAAUGUUCCGACUCCUGGCCUUU |
| 294 | 296 | 225 | 245 | GGCCAGGAGUCGGAACAUUGG | 746 | CCAAUGUUCCGACUCCUGGCCUU |
| 295 | 297 | 229 | 249 | AGGAGUCGGAACAUUGGCAUC | 747 | GAUGCCAAUGUUCCGACUCCUUU |
| 296 | 298 | 311 | 331 | CCAAUGUCCACCAGCUCAUCU | 748 | AGAUGAGCUGGUGGACAUUGGUU |
| 297 | 299 | 312 | 332 | CAAUGUCCACCAGCUCAUCUC | 749 | GAGAUGAGCUGGUGGACAUUGUU |
| 298 | 300 | 314 | 334 | AUGUCCACCAGCUCAUCUCCG | 750 | CGGAGAUGAGCUGGUGGACAUUU |
| 299 | 301 | 315 | 335 | UGUCCACCAGCUCAUCUCCGG | 751 | CCGGAGAUGAGCUGGUGGACAUU |
| 300 | 302 | 316 | 336 | GUCCACCAGCUCAUCUCCGGC | 752 | GCCGGAGAUGAGCUGGUGGACUU |
| 301 | 303 | 317 | 337 | UCCACCAGCUCAUCUCCGGCA | 753 | UGCCGGAGAUGAGCUGGUGGAUU |
| 302 | 304 | 319 | 339 | CACCAGCUCAUCUCCGGCAAA | 754 | UUUGCCGGAGAUGAGCUGGUGUU |
| 303 | 305 | 351 | 371 | UCUUACCAGAGUGUCUGAUGG | 755 | CCAUCAGACACUCUGGUAAGAUU |
| 304 | 306 | 352 | 372 | CUUACCAGAGUGUCUGAUGGG | 756 | CCCAUCAGACACUCUGGUAAGUU |
| 305 | 307 | 353 | 373 | UUACCAGAGUGUCUGAUGGGG | 757 | CCCCAUCAGACACUCUGGUAAUU |
| 306 | 308 | 354 | 374 | UACCAGAGUGUCUGAUGGGGA | 758 | UCCCCAUCAGACACUCUGGUAUU |
| 307 | 309 | 357 | 377 | CAGAGUGUCUGAUGGGGAAAA | 759 | UUUUCCCCAUCAGACACUCUGUU |
| 308 | 310 | 358 | 378 | AGAGUGUCUGAUGGGGAAAAC | 760 | GUUUUCCCCAUCAGACACUCUUU |
| 309 | 311 | 359 | 379 | GAGUGUCUGAUGGGGAAAACG | 761 | CGUUUUCCCCAUCAGACACUCUU |
| 310 | 312 | 360 | 380 | AGUGUCUGAUGGGGAAAACGU | 762 | ACGUUUUCCCCAUCAGACACUUU |
| 311 | 313 | 361 | 381 | GUGUCUGAUGGGGAAAACGUU | 763 | AACGUUUUCCCCAUCAGACACUU |
| 312 | 314 | 362 | 382 | UGUCUGAUGGGGAAAACGUUC | 764 | GAACGUUUUCCCCAUCAGACAUU |
| 313 | 315 | 363 | 383 | GUCUGAUGGGGAAAACGUUCU | 765 | AGAACGUUUUCCCCAUCAGACUU |
| 314 | 316 | 364 | 384 | UCUGAUGGGGAAAACGUUCUG | 766 | CAGAACGUUUUCCCCAUCAGAUU |
| 315 | 317 | 365 | 385 | CUGAUGGGGAAAACGUUCUGG | 767 | CCAGAACGUUUUCCCCAUCAGUU |
| 316 | 318 | 366 | 386 | UGAUGGGGAAAACGUUCUGGU | 768 | ACCAGAACGUUUUCCCCAUCAUU |
| 317 | 319 | 367 | 387 | GAUGGGGAAAACGUUCUGGUG | 769 | CACCAGAACGUUUUCCCCAUCUU |
| 318 | 320 | 369 | 389 | UGGGGAAAACGUUCUGGUGUC | 770 | GACACCAGAACGUUUUCCCCAUU |
| 319 | 321 | 371 | 391 | GGGAAAACGUUCUGGUGUCUG | 771 | CAGACACCAGAACGUUUUCCCUU |
| 320 | 322 | 372 | 392 | GGAAAACGUUCUGGUGUCUGA | 772 | UCAGACACCAGAACGUUUUCCUU |
| 321 | 323 | 375 | 395 | AAACGUUCUGGUGUCUGACUU | 773 | AAGUCAGACACCAGAACGUUUUU |
| 322 | 324 | 376 | 396 | AACGUUCUGGUGUCUGACUUU | 774 | AAAGUCAGACACCAGAACGUUUU |
| 323 | 325 | 377 | 397 | ACGUUCUGGUGUCUGACUUUC | 775 | GAAAGUCAGACACCAGAACGUUU |
| 324 | 326 | 378 | 398 | CGUUCUGGUGUCUGACUUUCG | 776 | CGAAAGUCAGACACCAGAACGUU |
| 325 | 327 | 382 | 402 | CUGGUGUCUGACUUUCGGUCC | 777 | GGACCGAAAGUCAGACACCAGUU |
| 326 | 328 | 383 | 403 | UGGUGUCUGACUUUCGGUCCA | 778 | UGGACCGAAAGUCAGACACCAUU |
| 327 | 329 | 384 | 404 | GGUGUCUGACUUUCGGUCCAA | 779 | UUGGACCGAAAGUCAGACACCUU |
| 328 | 330 | 385 | 405 | GUGUCUGACUUUCGGUCCAAA | 780 | UUUGGACCGAAAGUCAGACACUU |
| 329 | 331 | 387 | 407 | GUCUGACUUUCGGUCCAAAGA | 781 | UCUUUGGACCGAAAGUCAGACUU |
| 330 | 332 | 388 | 408 | UCUGACUUUCGGUCCAAAGAC | 782 | GUCUUUGGACCGAAAGUCAGAUU |
| 331 | 333 | 398 | 418 | GGUCCAAAGACGAAGUCGUGG | 783 | CCACGACUUCGUCUUUGGACCUU |
| 332 | 334 | 399 | 419 | GUCCAAAGACGAAGUCGUGGA | 784 | UCCACGACUUCGUCUUUGGACUU |
| 333 | 335 | 400 | 420 | UCCAAAGACGAAGUCGUGGAU | 785 | AUCCACGACUUCGUCUUUGGAUU |
| 334 | 336 | 402 | 422 | CAAAGACGAAGUCGUGGAUGC | 786 | GCAUCCACGACUUCGUCUUUGUU |
| 335 | 337 | 403 | 423 | AAAGACGAAGUCGUGGAUGCC | 787 | GGCAUCCACGACUUCGUCUUUUU |
| 336 | 338 | 404 | 424 | AAGACGAAGUCGUGGAUGCCU | 788 | AGGCAUCCACGACUUCGUCUUUU |
| 337 | 339 | 405 | 425 | AGACGAAGUCGUGGAUGCCUU | 789 | AAGGCAUCCACGACUUCGUCUUU |
| 338 | 340 | 407 | 427 | ACGAAGUCGUGGAUGCCUUGG | 790 | CCAAGGCAUCCACGACUUCGUUU |
| 339 | 341 | 408 | 428 | CGAAGUCGUGGAUGCCUUGGU | 791 | ACCAAGGCAUCCACGACUUCGUU |
| 340 | 342 | 409 | 429 | GAAGUCGUGGAUGCCUUGGUA | 792 | UACCAAGGCAUCCACGACUUCUU |
| 341 | 343 | 410 | 430 | AAGUCGUGGAUGCCUUGGUAU | 793 | AUACCAAGGCAUCCACGACUUUU |
| 342 | 344 | 412 | 432 | GUCGUGGAUGCCUUGGUAUGU | 794 | ACAUACCAAGGCAUCCACGACUU |
| 343 | 345 | 413 | 433 | UCGUGGAUGCCUUGGUAUGUU | 795 | AACAUACCAAGGCAUCCACGAUU |
| 344 | 346 | 415 | 435 | GUGGAUGCCUUGGUAUGUUCC | 796 | GGAACAUACCAAGGCAUCCACUU |
| 345 | 347 | 416 | 436 | UGGAUGCCUUGGUAUGUUCCU | 797 | AGGAACAUACCAAGGCAUCCAUU |
| 346 | 348 | 417 | 437 | GGAUGCCUUGGUAUGUUCCUG | 798 | CAGGAACAUACCAAGGCAUCCUU |
| 347 | 349 | 419 | 439 | AUGCCUUGGUAUGUUCCUGCU | 799 | AGCAGGAACAUACCAAGGCAUUU |
| 348 | 350 | 421 | 441 | GCCUUGGUAUGUUCCUGCUUC | 800 | GAAGCAGGAACAUACCAAGGCUU |
| 349 | 351 | 430 | 450 | UGUUCCUGCUUCAUGCCCUUC | 801 | GAAGGGCAUGAAGCAGGAACAUU |
| 350 | 352 | 433 | 453 | UCCUGCUUCAUGCCCUUCUAC | 802 | GUAGAAGGGCAUGAAGCAGGAUU |
| 351 | 353 | 454 | 474 | AGUGGCCUUAUCCCUCCUUCC | 803 | GGAAGGAGGGAUAAGGCCACUUU |
| 352 | 354 | 457 | 477 | GGCCUUAUCCCUCCUUCCUUC | 804 | GAAGGAAGGAGGGAUAAGGCCUU |
| 353 | 355 | 459 | 479 | CCUUAUCCCUCCUUCCUUCAG | 805 | CUGAAGGAAGGAGGGAUAAGGUU |
| 354 | 356 | 460 | 480 | CUUAUCCCUCCUUCCUUCAGA | 806 | UCUGAAGGAAGGAGGGAUAAGUU |
| 355 | 357 | 461 | 481 | UUAUCCCUCCUUCCUUCAGAG | 807 | CUCUGAAGGAAGGAGGGAUAAUU |
| 356 | 358 | 465 | 485 | CCCUCCUUCCUUCAGAGGCGU | 808 | ACGCCUCUGAAGGAAGGAGGGUU |
| 357 | 359 | 466 | 486 | CCUCCUUCCUUCAGAGGCGUG | 809 | CACGCCUCUGAAGGAAGGAGGUU |
| 358 | 360 | 467 | 487 | CUCCUUCCUUCAGAGGCGUGC | 810 | GCACGCCUCUGAAGGAAGGAGUU |
| 359 | 361 | 469 | 489 | CCUUCCUUCAGAGGCGUGCGA | 811 | UCGCACGCCUCUGAAGGAAGGUU |
| 360 | 362 | 470 | 490 | CUUCCUUCAGAGGCGUGCGAU | 812 | AUCGCACGCCUCUGAAGGAAGUU |
| 361 | 363 | 471 | 491 | UUCCUUCAGAGGCGUGCGAUA | 813 | UAUCGCACGCCUCUGAAGGAAUU |
| 362 | 364 | 489 | 509 | AUAUGUGGAUGGAGGAGUGAG | 814 | CUCACUCCUCCAUCCACAUAUUU |
| 363 | 365 | 499 | 519 | GGAGGAGUGAGUGACAACGUA | 815 | UACGUUGUCACUCACUCCUCCUU |
| 364 | 366 | 501 | 521 | AGGAGUGAGUGACAACGUACC | 816 | GGUACGUUGUCACUCACUCCUUU |
| 365 | 367 | 502 | 522 | GGAGUGAGUGACAACGUACCC | 817 | GGGUACGUUGUCACUCACUCCUU |
| 366 | 368 | 505 | 525 | GUGAGUGACAACGUACCCUUC | 818 | GAAGGGUACGUUGUCACUCACUU |
| 367 | 369 | 528 | 548 | UGAUGCCAAAACAACCAUCAC | 819 | GUGAUGGUUGUUUUGGCAUCAUU |
| 368 | 370 | 529 | 549 | GAUGCCAAAACAACCAUCACC | 820 | GGUGAUGGUUGUUUUGGCAUCUU |
| 369 | 371 | 530 | 550 | AUGCCAAAACAACCAUCACCG | 821 | CGGUGAUGGUUGUUUUGGCAUUU |
| 370 | 372 | 533 | 553 | CCAAAACAACCAUCACCGUGU | 822 | ACACGGUGAUGGUUGUUUUGGUU |
| 371 | 373 | 534 | 554 | CAAAACAACCAUCACCGUGUC | 823 | GACACGGUGAUGGUUGUUUUGUU |
| 372 | 374 | 536 | 556 | AAACAACCAUCACCGUGUCCC | 824 | GGGACACGGUGAUGGUUGUUUUU |
| 373 | 375 | 537 | 557 | AACAACCAUCACCGUGUCCCC | 825 | GGGGACACGGUGAUGGUUGUUUU |
| 374 | 376 | 538 | 558 | ACAACCAUCACCGUGUCCCCC | 826 | GGGGGACACGGUGAUGGUUGUUU |
| 375 | 377 | 539 | 559 | CAACCAUCACCGUGUCCCCCU | 827 | AGGGGGACACGGUGAUGGUUGUU |
| 376 | 378 | 544 | 564 | AUCACCGUGUCCCCCUUCUAU | 828 | AUAGAAGGGGGACACGGUGAUUU |
| 377 | 379 | 545 | 565 | UCACCGUGUCCCCCUUCUAUG | 829 | CAUAGAAGGGGGACACGGUGAUU |
| 378 | 380 | 546 | 566 | CACCGUGUCCCCCUUCUAUGG | 830 | CCAUAGAAGGGGGACACGGUGUU |
| 379 | 381 | 547 | 567 | ACCGUGUCCCCCUUCUAUGGG | 831 | CCCAUAGAAGGGGGACACGGUUU |
| 380 | 382 | 550 | 570 | GUGUCCCCCUUCUAUGGGGAG | 832 | CUCCCCAUAGAAGGGGGACACUU |
| 381 | 383 | 551 | 571 | UGUCCCCCUUCUAUGGGGAGU | 833 | ACUCCCCAUAGAAGGGGGACAUU |
| 382 | 384 | 552 | 572 | GUCCCCCUUCUAUGGGGAGUA | 834 | UACUCCCCAUAGAAGGGGGACUU |
| 383 | 385 | 553 | 573 | UCCCCCUUCUAUGGGGAGUAC | 835 | GUACUCCCCAUAGAAGGGGGAUU |
| 384 | 386 | 556 | 576 | CCCUUCUAUGGGGAGUACGAC | 836 | GUCGUACUCCCCAUAGAAGGGUU |
| 385 | 387 | 557 | 577 | CCUUCUAUGGGGAGUACGACA | 837 | UGUCGUACUCCCCAUAGAAGGUU |
| 386 | 388 | 559 | 579 | UUCUAUGGGGAGUACGACAUC | 838 | GAUGUCGUACUCCCCAUAGAAUU |
| 387 | 389 | 560 | 580 | UCUAUGGGGAGUACGACAUCU | 839 | AGAUGUCGUACUCCCCAUAGAUU |
| 388 | 390 | 561 | 581 | CUAUGGGGAGUACGACAUCUG | 840 | CAGAUGUCGUACUCCCCAUAGUU |
| 389 | 391 | 562 | 582 | UAUGGGGAGUACGACAUCUGC | 841 | GCAGAUGUCGUACUCCCCAUAUU |
| 390 | 392 | 572 | 592 | ACGACAUCUGCCCUAAAGUCA | 842 | UGACUUUAGGGCAGAUGUCGUUU |
| 391 | 393 | 573 | 593 | CGACAUCUGCCCUAAAGUCAA | 843 | UUGACUUUAGGGCAGAUGUCGUU |
| 392 | 394 | 574 | 594 | GACAUCUGCCCUAAAGUCAAG | 844 | CUUGACUUUAGGGCAGAUGUCUU |
| 393 | 395 | 575 | 595 | ACAUCUGCCCUAAAGUCAAGU | 845 | ACUUGACUUUAGGGCAGAUGUUU |
| 394 | 396 | 576 | 596 | CAUCUGCCCUAAAGUCAAGUC | 846 | GACUUGACUUUAGGGCAGAUGUU |
| 395 | 397 | 577 | 597 | AUCUGCCCUAAAGUCAAGUCC | 847 | GGACUUGACUUUAGGGCAGAUUU |
| 396 | 398 | 582 | 602 | CCCUAAAGUCAAGUCCACGAA | 848 | UUCGUGGACUUGACUUUAGGGUU |
| 397 | 399 | 583 | 603 | CCUAAAGUCAAGUCCACGAAC | 849 | GUUCGUGGACUUGACUUUAGGUU |
| 398 | 400 | 585 | 605 | UAAAGUCAAGUCCACGAACUU | 850 | AAGUUCGUGGACUUGACUUUAUU |
| 399 | 401 | 586 | 606 | AAAGUCAAGUCCACGAACUUU | 851 | AAAGUUCGUGGACUUGACUUUUU |
| 400 | 402 | 587 | 607 | AAGUCAAGUCCACGAACUUUC | 852 | GAAAGUUCGUGGACUUGACUUUU |
| 401 | 403 | 588 | 608 | AGUCAAGUCCACGAACUUUCU | 853 | AGAAAGUUCGUGGACUUGACUUU |
| 402 | 404 | 589 | 609 | GUCAAGUCCACGAACUUUCUU | 854 | AAGAAAGUUCGUGGACUUGACUU |
| 403 | 405 | 590 | 610 | UCAAGUCCACGAACUUUCUUC | 855 | GAAGAAAGUUCGUGGACUUGAUU |
| 404 | 406 | 591 | 611 | CAAGUCCACGAACUUUCUUCA | 856 | UGAAGAAAGUUCGUGGACUUGUU |
| 405 | 407 | 592 | 612 | AAGUCCACGAACUUUCUUCAU | 857 | AUGAAGAAAGUUCGUGGACUUUU |
| 406 | 408 | 593 | 613 | AGUCCACGAACUUUCUUCAUG | 858 | CAUGAAGAAAGUUCGUGGACUUU |
| 407 | 409 | 595 | 615 | UCCACGAACUUUCUUCAUGUG | 859 | CACAUGAAGAAAGUUCGUGGAUU |
| 408 | 410 | 618 | 638 | CAUCACCAAGCUCAGUCUACG | 860 | CGUAGACUGAGCUUGGUGAUGUU |
| 409 | 411 | 619 | 639 | AUCACCAAGCUCAGUCUACGC | 861 | GCGUAGACUGAGCUUGGUGAUUU |
| 410 | 412 | 620 | 640 | UCACCAAGCUCAGUCUACGCC | 862 | GGCGUAGACUGAGCUUGGUGAUU |
| 411 | 413 | 621 | 641 | CACCAAGCUCAGUCUACGCCU | 863 | AGGCGUAGACUGAGCUUGGUGUU |
| 412 | 414 | 622 | 642 | ACCAAGCUCAGUCUACGCCUC | 864 | GAGGCGUAGACUGAGCUUGGUUU |
| 413 | 415 | 623 | 643 | CCAAGCUCAGUCUACGCCUCU | 865 | AGAGGCGUAGACUGAGCUUGGUU |
| 414 | 416 | 624 | 644 | CAAGCUCAGUCUACGCCUCUG | 866 | CAGAGGCGUAGACUGAGCUUGUU |
| 415 | 417 | 628 | 648 | CUCAGUCUACGCCUCUGCACA | 867 | UGUGCAGAGGCGUAGACUGAGUU |
| 416 | 418 | 629 | 649 | UCAGUCUACGCCUCUGCACAG | 868 | CUGUGCAGAGGCGUAGACUGAUU |
| 417 | 419 | 630 | 650 | CAGUCUACGCCUCUGCACAGG | 869 | CCUGUGCAGAGGCGUAGACUGUU |
| 418 | 420 | 636 | 656 | ACGCCUCUGCACAGGGAACCU | 870 | AGGUUCCCUGUGCAGAGGCGUUU |
| 419 | 421 | 637 | 657 | CGCCUCUGCACAGGGAACCUC | 871 | GAGGUUCCCUGUGCAGAGGCGUU |
| 420 | 422 | 638 | 658 | GCCUCUGCACAGGGAACCUCU | 872 | AGAGGUUCCCUGUGCAGAGGCUU |
| 421 | 423 | 640 | 660 | CUCUGCACAGGGAACCUCUAC | 873 | GUAGAGGUUCCCUGUGCAGAGUU |
| 422 | 424 | 641 | 661 | UCUGCACAGGGAACCUCUACC | 874 | GGUAGAGGUUCCCUGUGCAGAUU |
| 423 | 425 | 644 | 664 | GCACAGGGAACCUCUACCUUC | 875 | GAAGGUAGAGGUUCCCUGUGCUU |
| 424 | 426 | 645 | 665 | CACAGGGAACCUCUACCUUCU | 876 | AGAAGGUAGAGGUUCCCUGUGUU |
| 425 | 427 | 694 | 714 | AAGGUGCUGGGAGAGAUAUGC | 877 | GCAUAUCUCUCCCAGCACCUUUU |
| 426 | 428 | 695 | 715 | AGGUGCUGGGAGAGAUAUGCC | 878 | GGCAUAUCUCUCCCAGCACCUUU |
| 427 | 429 | 696 | 716 | GGUGCUGGGAGAGAUAUGCCU | 879 | AGGCAUAUCUCUCCCAGCACCUU |
| 428 | 430 | 697 | 717 | GUGCUGGGAGAGAUAUGCCUU | 880 | AAGGCAUAUCUCUCCCAGCACUU |
| 429 | 431 | 735 | 755 | AUUCAGGUUCUUGGAAGAGAA | 881 | UUCUCUUCCAAGAACCUGAAUUU |
| 430 | 432 | 741 | 761 | GUUCUUGGAAGAGAAGGGCAU | 882 | AUGCCCUUCUCUUCCAAGAACUU |
| 431 | 433 | 742 | 762 | UUCUUGGAAGAGAAGGGCAUC | 883 | GAUGCCCUUCUCUUCCAAGAAUU |
| 432 | 434 | 743 | 763 | UCUUGGAAGAGAAGGGCAUCU | 884 | AGAUGCCCUUCUCUUCCAAGAUU |
| 433 | 435 | 744 | 764 | CUUGGAAGAGAAGGGCAUCUG | 885 | CAGAUGCCCUUCUCUUCCAAGUU |
| 434 | 436 | 745 | 765 | UUGGAAGAGAAGGGCAUCUGC | 886 | GCAGAUGCCCUUCUCUUCCAAUU |
| 435 | 437 | 747 | 767 | GGAAGAGAAGGGCAUCUGCAA | 887 | UUGCAGAUGCCCUUCUCUUCCUU |
| 436 | 438 | 748 | 768 | GAAGAGAAGGGCAUCUGCAAC | 888 | GUUGCAGAUGCCCUUCUCUUCUU |
| 437 | 439 | 749 | 769 | AAGAGAAGGGCAUCUGCAACA | 889 | UGUUGCAGAUGCCCUUCUCUUUU |
| 438 | 440 | 752 | 772 | AGAAGGGCAUCUGCAACAGGC | 890 | GCCUGUUGCAGAUGCCCUUCUUU |
| 439 | 441 | 753 | 773 | GAAGGGCAUCUGCAACAGGCC | 891 | GGCCUGUUGCAGAUGCCCUUCUU |
| 440 | 442 | 758 | 778 | GCAUCUGCAACAGGCCCCAGC | 892 | GCUGGGGCCUGUUGCAGAUGCUU |
| 441 | 443 | 1026 | 1046 | UUGCAACUUGCUACCCAUUAG | 893 | CUAAUGGGUAGCAAGUUGCAAUU |
| 442 | 444 | 1039 | 1059 | CCCAUUAGGAUAAUGUCUUAU | 894 | AUAAGACAUUAUCCUAAUGGGUU |
| 443 | 445 | 1062 | 1082 | AAUGCUGCCCUGUACCCUGCC | 895 | GGCAGGGUACAGGGCAGCAUUUU |
| 444 | 446 | 1067 | 1087 | UGCCCUGUACCCUGCCUGUGG | 896 | CCACAGGCAGGGUACAGGGCAUU |
| 445 | 447 | 1068 | 1088 | GCCCUGUACCCUGCCUGUGGA | 897 | UCCACAGGCAGGGUACAGGGCUU |
| 446 | 448 | 1077 | 1097 | CCUGCCUGUGGAAUCUGCCAU | 898 | AUGGCAGAUUCCACAGGCAGGUU |
| 447 | 449 | 1080 | 1100 | GCCUGUGGAAUCUGCCAUUGC | 899 | GCAAUGGCAGAUUCCACAGGCUU |
| 448 | 450 | 1159 | 1179 | CAGUGGGUGACCUCACAGGUG | 900 | CACCUGUGAGGUCACCCACUGUU |
| 449 | 451 | 1195 | 1215 | AUGUGUCUGCUCCCCGCCUCC | 901 | GGAGGCGGGGAGCAGACACAUUU |
| 450 | 452 | 1196 | 1216 | UGUGUCUGCUCCCCGCCUCCA | 902 | UGGAGGCGGGGAGCAGACACAUU |
| 451 | 2068 | 419 | 437 | AUGCCUUGGUAUGUUCCUG | 2108 | CAGGAACAUACCAAGGCAUUU |
| 452 | 2069 | 419 | 437 | AUGCCUUGGUAUGUUCCUG | 2109 | AAGGAACAUACCAAGGCAUUU |
| 453 | 2070 | 419 | 437 | AUGCCUUGGUAUGUUCCUG | 2110 | AAGGAACAUACCAAGGCAUUU |
| 454 | 2071 | 419 | 437 | AUGCCUUGGUAUGUUCCUG | 2111 | UAGGAACAUACCAAGGCAUUU |
| 455 | 2072 | 419 | 437 | AUGCCUUGGUAUGUUCCUG | 2112 | UAGGAACAUACCAAGGCAUUU |
| 456 | 2073 | 419 | 437 | AUGCCUUGGUAUGUUCCUG | 2113 | CAGGAACAUACCAAGGCAUCC |
| 457 | 2074 | 419 | 437 | AUGCCUUGGUAUGUUCCUG | 2114 | AAGGAACAUACCAAGGCAUCC |
| 458 | 2075 | 419 | 439 | AUGCCUUGGUAUGUUCCUGCU | 2115 | AGCAGGAACAUACCAAGGCAUCC |
| 459 | 2076 | 417 | 437 | GGAUGCCUUGGUAUGUUCCUG | 2116 | AAGGAACAUACCAAGGCAUCCAC |
| 460 | 2077 | 419 | 437 | AUGCCUUGAUAUGUUCCUG | 2117 | UAGGAACAUAUCAAGGCAUUU |
| 461 | 2078 | 419 | 437 | AUGCCUUGGUUUGUUCCUG | 2118 | UAGGAACAAACCAAGGCAUUU |
| 462 | 2079 | 419 | 437 | AUGCCUUGGUUUGUUCCUG | 2119 | AAGGAACAAACCAAGGCAUCC |
| 463 | 2080 | 419 | 437 | AUGCCUUGGUAUGUUCCUG | 2120 | CAGGAACAUACCAAGGCAUUU |
| 464 | 2081 | 419 | 437 | AUGCCUUGGUAUGUUCCUG | 2121 | CAGGAACAUACCAAGGCAUUU |
| 465 | 2082 | 419 | 437 | AUGCCUUGGUAUGUUCCUG | 2122 | CAGGAACAUACCAAGGCAUUU |
| 468 | 2085 | 419 | 437 | AUGCCUUGGUAUGUUCCUG | 2125 | AAGGAACAUACCAAGGCAUCC |
| 472 | 2089 | 419 | 437 | AUGCCUUGGUAUGUUCCUG | 2129 | AAGGAACAUACCAAGGCAUCC |
| 473 | 2090 | 419 | 437 | AUGCCUUGGUAUGUUCCUG | 2130 | UAGGAACAUACCAAGGCAUCC |
| 474 | 2091 | 417 | 437 | GGAUGCCUUGGUAUGUUCCUG | 2131 | AAGGAACAUACCAAGGCAUCCAC |
| 475 | 2092 | 419 | 437 | AUGCCUUGGUUUGUUCCUG | 2132 | AAGGAACAAACCAAGGCAUCC |
| 476 | 2093 | 385 | 405 | GUGUCUGACUUUCGGUCCAAA | 2133 | UUUGGACCGAAAGUCAGACACUU |
| 477 | 2094 | 385 | 405 | GUGUCUGACUUUCGGUCCAAA | 2134 | UUUGGACCGAAAGUCAGACACCA |
| 478 | 2095 | 385 | 405 | GUGUCUGACUUUCGGUCCAAA | 2135 | AUUGGACCGAAAGUCAGACACCA |
| 479 | 2096 | 385 | 405 | GUGUCUGACUUUCGGUCCAAA | 2136 | AUUGGACCGAAAGUCAGACACCA |
| 480 | 2097 | 400 | 420 | UCCAAAGACGAAGUCGUGGAU | 2137 | AUCCACGACUUCGUCUUUGGACC |
| 481 | 2098 | 400 | 420 | UCCAAAGACGAAGUCGUGGAU | 2138 | UUCCACGACUUCGUCUUUGGACC |
| 482 | 2099 | 400 | 420 | UCCAAAGACGAAGUCGUGGAU | 2139 | UUCCACGACUUCGUCUUUGGACC |
| 483 | 2100 | 530 | 550 | AUGCCAAAACAACCAUCACCG | 2140 | AGGUGAUGGUUGUUUUGGCAUUU |
| 484 | 2101 | 530 | 550 | AUGCCAAAACAACCAUCACCG | 2141 | AGGUGAUGGUUGUUUUGGCAUCA |
| 485 | 2102 | 530 | 550 | AUGCCAAAACAACCAUCACCG | 2142 | AGGUGAUGGUUGUUUUGGCAUCA |
| 486 | 2103 | 409 | 429 | GAAGUCGUGGAUGCCUUGGUA | 2143 | UACCAAGGCAUCCACGACUUCGU |
| 487 | 2104 | 409 | 429 | GAAGUCGUGGAUGCCUUGGUA | 2144 | AACCAAGGCAUCCACGACUUCGU |
| 488 | 2105 | 409 | 429 | GAAGUCGUGGAUGCCUUGGUA | 2145 | AACCAAGGCAUCCACGACUUCGU |
| 489 | 2106 | 413 | 433 | UCGUGGAUGCCUUGGUAUGUU | 2146 | AACAUACCAAGGCAUCCACGACU |
| 490 | 2107 | 411 | 431 | AGUCGUGGAUGCCUUGGUAUG | 2147 | AAUACCAAGGCAUCCACGACUUC |
| 491 | 2228 | 400 | 420 | UCCAAAGACGAAGUCGUGGAU | 2253 | AUCCACGACUUCGUCUUUGGAUU |
| 494 | 2231 | 530 | 550 | AUGCCAAAACAACCAUCACCU | 2256 | AGGUGAUGGUUGUUUUGGCAUCA |
| 496 | 2233 | 409 | 429 | GAAGUCGUGGAUGCCUUGGUA | 2258 | UACCAAGGCAUCCACGACUUCGU |
| 497 | 2234 | 409 | 429 | GAAGUCGUGGAUGCCUUGGUA | 2259 | AACCAAGGCAUCCACGACUUCGU |
| 498 | 2235 | 409 | 429 | GAAGUCGUGGAUGCCUUGGUU | 2260 | AACCAAGGCAUCCACGACUUCGU |
| 499 | 2236 | 409 | 429 | GAAGUCGUGGCUGCCUUGGUA | 2261 | AACCAAGGCAUCCACGACUUCGU |
| 501 | 2238 | 409 | 429 | GAAGUCGUGGAUGCCUUGGUU | 2263 | AACCAAGGCAUCCACGACUUCGU |
| 502 | 2239 | 409 | 429 | GAAGUCGUGGCUGCCUUGGUA | 2264 | AACCAAGGCAUCCACGACUUCGU |
| 504 | 2241 | 412 | 432 | GUCGUGGAUGCCUUGGUAUGU | 2266 | ACAUACCAAGGCAUCCACGACUU |
| 505 | 2242 | 413 | 433 | UCGUGGAUGCCUUGGUAUGUU | 2267 | AACAUACCAAGGCAUCCACGAUU |
| 506 | 2243 | 413 | 433 | UCGUGGAUGCCUUGGUAUGUU | 2268 | AACAUACCAAGGCAUCCACGAUU |
| 507 | 2244 | 497 | 515 | AUGGAGGAGUGAGUGACAA | 2269 | UUGUCACUCACUCCUCCAUUU |
| 508 | 2245 | 530 | 550 | AUGCCAAAACAACCAUCACCG | 2270 | CGGUGAUGGUUGUUUUGGCAUUU |
| 509 | 2246 | 409 | 429 | GAAGUCGUGGAUGCCUUGGUA | 2271 | UACCAAGGCAUCCACGACUUCUU |
| 510 | 2247 | 409 | 429 | GAAGUCGUGGAUGCCUUGGUA | 2272 | UACCAAGGCAUCCACGACUUCUU |
| 511 | 2248 | 385 | 405 | GUGUCUGACUUUCGGUCCAAA | 2273 | UUUGGACCGAAAGUCAGACACUU |
| 512 | 2249 | 400 | 420 | UCCAAAGACGAAGUCGUGGAU | 2274 | AUCCACGACUUCGUCUUUGGAUU |
| 513 | 2250 | 412 | 432 | GUCGUGGAUGCCUUGGUAUGU | 2275 | ACAUACCAAGGCAUCCACGACUU |
| 514 | 2251 | 530 | 550 | AUGCCAAAACAACCAUCACCG | 2276 | CGGUGAUGGUUGUUUUGGCAUUU |
| 515 | 2252 | 409 | 429 | GAAGUCGUGGAUGCCUUGGUA | 2277 | UACCAAGGCAUCCACGACUUCUU |

TABLE 1A
siRNA Sequences

| | | Target | Target | | | |
|---|---|---|---|---|---|---|
| | | Site | Site | | | |
| | | Start | End | | | |
| siRNA | | Position | Position | Sense Strand Base | | Antisense Strand Base |
| Duplex | SEQ | in SEQ | in SEQ | Sequence + Chem | SEQ | Sequence + Chem |
| ID NO. | ID | ID NO. | ID NO. | Modifications | ID | Modifications |
| (Dx) | NO. | 2067 | 2067 | (5′-3′) | NO. | (5′-3′) |
| 466 | 2083 | 419 | 437 | AUGCf2PUUGGUAUGUUCCUG | 2123 | CAGGAACAUACCAAGGCAUUU |
| 467 | 2084 | 419 | 437 | AUGCCUUGGUAUGUUCmun34CUG | 2124 | CAGGAACAUACCAAGGCAUCC |
| 469 | 2086 | 419 | 437 | AUGCf2PUUGGUAUGUUCCUG | 2126 | AAGGAACAUACCAAGGCAUCC |
| 470 | 2087 | 419 | 437 | AUGCf2PUUGGUAUGUUCCUG | 2127 | AAGGAACAUACCAAGGCAUCC |
| 471 | 2088 | 419 | 437 | AUGCCUUGGUAUGUUCmun34CUG | 2128 | AAGGAACAUACCAAGGCAUCC |
| 492 | 2229 | 530 | 550 | AUGCCAAAAf2PAACCAUCACCG | 2254 | AGGUGAUGGUUGUUUUGGCAUCA |
| 493 | 2230 | 530 | 550 | AUGCCAAAACAACCAUCAmun34CCG | 2255 | AGGUGAUGGUUGUUUUGGCAUCA |
| 495 | 2232 | 530 | 550 | AUGCCAAAAf2PAACCAUCACCU | 2257 | AGGUGAUGGUUGUUUUGGCAUCA |
| 500 | 2237 | 409 | 429 | GAAGUCGUGGAUGCCUUGmun34GUA | 2262 | AACCAAGGCAUCCACGACUUCGU |
| 503 | 2240 | 409 | 429 | GAAGUCGUGGAUGCCUUGmun34GUA | 2265 | AACCAAGGCAUCCACGACUUCGU |

TABLE 2
siRNA Modified Sequences

| | | Target | | | | |
|---|---|---|---|---|---|---|
| | | Site | Target | | | |
| | | Start | Site End | | | |
| siRNA | | Position | Position | | | |
| Duplex | | in SEQ | in SEQ | | | |
| ID NO. | SEQ ID | ID NO. | ID NO. | Sense Strand Base Sequence + Chem | SEQ ID | Antisense Strand Base Sequence + Chem |
| (MDx) | NO. | 2067 | 2067 | Modifications (5′-3′) | NO. | Modifications (5′-3′) |
| 1 | 903 | 1 | 19 | fApsmUpsfGmUfAmCfGmAfCmGfCmAfGmAfG | 1485 | mCpsfGpsmCfGmCfUmCfUmGfCmGfUmCfGmUfAmCfAmUpsmUp |
| | | | | mCfGmCfGpsmUpsmU | | smU |
| 2 | 904 | 2 | 20 | fUpsmGpsfUmAfCmGfAmCfGmCfAmGfAmGfC | 1486 | mCpsfCpsmGfCmGfCmUfCmUfGmCfGmUfCmGfUmAfCmApsmUp |
| | | | | mGfCmGfGpsmUpsmU | | smU |
| 3 | 905 | 3 | 21 | fGpsmUpsfAmCfGmAfCmGfCmAfGmAfGmCfG | 1487 | mGpsfCpsmCfGmCfGmCfUmCfUmGfCmGfUmCfGmUfAmCpsmUp |
| | | | | mCfGmGfCpsmUpsmU | | smU |
| 4 | 906 | 5 | 23 | fApsmCpsfGmAfCmGfCmAfGmAfGmCfGmCfG | 1488 | mCpsfApsmGfCmCfGmCfGmCfUmCfUmGfCmGfUmCfGmUpsmUp |
| | | | | mGfCmUfGpsmUpsmU | | smU |
| 5 | 907 | 6 | 24 | fCpsmGpsfAmCfGmCfAmGfAmGfCmGfCmGfG | 1489 | mCpsfCpsmAfGmCfCmGfCmGfCmUfCmUfGmCfGmUfCmGpsmUp |
| | | | | mCfUmGfGpsmUpsmU | | smU |
| 6 | 908 | 7 | 25 | fGpsmApsfCmGfCmAfGmAfGmCfGmCfGmGfC | 1490 | mUpsfCpsmCfAmGfCmCfGmCfGmCfUmCfUmGfCmGfUmCpsmUp |
| | | | | mUfGmGfApsmUpsmU | | smU |
| 7 | 909 | 10 | 28 | fGpsmCpsfAmGfAmGfCmGfCmGfGmCfUmGf | 1491 | mApsfGpsmCfUmCfCmAfGmCfCmGfCmGfCmUfCmUfGmCpsmUp |
| | | | | GmAfGmCfUpsmUpsmU | | smU |
| 8 | 910 | 11 | 29 | fCpsmApsfGmAfGmCfGmCfGmGfCmUfGmGfA | 1492 | mApsfApsmGfCmUfCmCfAmGfCmCfGmCfGmCfUmCfUmGpsmUp |
| | | | | mGfCmUfUpsmUpsmU | | smU |
| 9 | 911 | 12 | 30 | fApsmGpsfAmGfCmGfCmGfGmCfUmGfGmAf | 1493 | mCpsfApsmAfGmCfUmCfCmAfGmCfCmGfCmGfCmUfCmUpsmUp |
| | | | | GmCfUmUfGpsmUpsmU | | smU |
| 10 | 912 | 13 | 31 | fGpsmApsfGmCfGmCfGmGfCmUfGmGfAmGf | 1494 | mApsfCpsmAfAmGfCmUfCmCfAmGfCmCfGmCfGmCfUmCpsmUps |
| | | | | CmUfUmGfUpsmUpsmU | | mU |
| 11 | 913 | 15 | 33 | fGpsmCpsfGmCfGmGfCmUfGmGfAmGfCmUf | 1495 | mGpsfGpsmAfCmAfAmGfCmUfCmCfAmGfCmCfGmCfGmCpsmUp |
| | | | | UmGfUmCfCpsmUpsmU | | smU |
| 12 | 914 | 16 | 34 | fCpsmGpsfCmGfGmCfUmGfGmAfGmCfUmUf | 1496 | mApsfGpsmGfAmCfAmAfGmCfUmCfCmAfGmCfCmGfCmGpsmUp |
| | | | | GmUfCmCfUpsmUpsmU | | smU |
| 13 | 915 | 17 | 35 | fGpsmCpsfGmGfCmUfGmGfAmGfCmUfUmGf | 1497 | mApsfApsmGfGmAfCmAfAmGfCmUfCmCfAmGfCmCfGmCpsmUp |
| | | | | UmCfCmUfUpsmUpsmU | | smU |
| 14 | 916 | 20 | 38 | fGpsmCpsfUmGfGmAfGmCfUmUfGmUfCmCf | 1498 | mGpsfCpsmGfAmAfGmGfAmCfAmAfGmCfUmCfCmAfGmCpsmUp |
| | | | | UmUfCmGfCpsmUpsmU | | smU |
| 15 | 917 | 22 | 40 | fUpsmGpsfGmAfGmCfUmUfGmUfCmCfUmUf | 1499 | mCpsfCpsmGfCmGfAmAfGmGfAmCfAmAfGmCfUmCfCmApsmUp |
| | | | | CmGfCmGfGpsmUpsmU | | smU |
| 16 | 918 | 23 | 41 | fGpsmGpsfAmGfCmUfUmGfUmCfCmUfUmCf | 1500 | mCpsfCpsmCfGmCfGmAfAmGfGmAfCmAfAmGfCmUfCmCpsmUps |
| | | | | GmCfGmGfGpsmUpsmU | | mU |
| 17 | 919 | 24 | 42 | fGpsmApsfGmCfUmUfGmUfCmCfUmUfCmGf | 1501 | mGpsfCpsmCfCmGfCmGfAmAfGmGfAmCfAmAfGmCfUmCpsmUp |
| | | | | CmGfGmGfCpsmUpsmU | | smU |
| 18 | 920 | 25 | 43 | fApsmGpsfCmUfUmGfUmCfCmUfUmCfGmCf | 1502 | mApsfGpsmCfCmCfGmCfGmAfAmGfGmAfCmAfAmGfCmUpsmUp |
| | | | | GmGfGmCfUpsmUpsmU | | smU |
| 19 | 921 | 30 | 48 | fGpsmUpsfCmCfUmUfCmGfCmGfGmGfCmUf | 1503 | mGpsfCpsmCfGmCfAmGfCmCfCmGfCmGfAmAfGmGfAmCpsmUp |
| | | | | GmCfGmGfCpsmUpsmU | | smU |
| 20 | 922 | 41 | 59 | fGpsmCpsfUmGfCmGfGmCfUmUfCmCfUmGf | 1504 | mApsfApsmGfCmCfCmAfGmGfAmAfGmCfCmGfCmAfGmCpsmUp |
| | | | | GmGfCmUfUpsmUpsmU | | smU |
| 21 | 923 | 42 | 60 | fCpsmUpsfGmCfGmGfCmUfUmCfCmUfGmGf | 1505 | mGpsfApsmAfGmCfCmCfAmGfGmAfAmGfCmCfGmCfAmGpsmUp |
| | | | | GmCfUmUfCpsmUpsmU | | smU |
| 22 | 924 | 45 | 63 | fCpsmGpsfGmCfUmUfCmCfUmGfGmGfCmUf | 1506 | mGpsfUpsmAfGmAfAmGfCmCfCmAfGmGfAmAfGmCfCmGpsmUp |
| | | | | UmCfUmAfCpsmUpsmU | | smU |
| 23 | 925 | 52 | 70 | fCpsmUpsfGmGfGmCfUmUfCmUfAmCfCmAfC | 1507 | mCpsfGpsmAfCmGfUmGfGmUfAmGfAmAfGmCfCmCfAmGpsmUp |
| | | | | mGfUmCfGpsmUpsmU | | smU |
| 24 | 926 | 53 | 71 | fUpsmGpsfGmGfCmUfUmCfUmAfCmCfAmCfG | 1508 | mCpsfCpsmGfAmCfGmUfGmGfUmAfGmAfAmGfCmCfCmApsmUp |
| | | | | mUfCmGfGpsmUpsmU | | smU |
| 25 | 927 | 55 | 73 | fGpsmGpsfCmUfUmCfUmAfCmCfAmCfGmUfC | 1509 | mCpsfCpsmCfCmGfAmCfGmUfGmGfUmAfGmAfAmGfCmCpsmUp |
| | | | | mGfGmGfGpsmUpsmU | | smU |
| 26 | 928 | 56 | 74 | fGpsmCpsfUmUfCmUfAmCfCmAfCmGfUmCfG | 1510 | mGpsfCpsmCfCmCfGmAfCmGfUmGfGmUfAmGfAmAfGmCpsmUp |
| | | | | mGfGmGfCpsmUpsmU | | smU |
| 27 | 929 | 57 | 75 | fCpsmUpsfUmCfUmAfCmCfAmCfGmUfCmGfG | 1511 | mCpsfGpsmCfCmCfCmGfAmCfGmUfGmGfUmAfGmAfAmGpsmUp |
| | | | | mGfGmCfGpsmUpsmU | | smU |
| 28 | 930 | 58 | 76 | fUpsmUpsfCmUfAmCfCmAfCmGfUmCfGmGfG | 1512 | mUpsfCpsmGfCmCfCmCfGmAfCmGfUmGfGmUfAmGfAmApsmUp |
| | | | | mGfCmGfApsmUpsmU | | smU |
| 29 | 931 | 59 | 77 | fUpsmCpsfUmAfCmCfAmCfGmUfCmGfGmGfG | 1513 | mGpsfUpsmCfGmCfCmCfCmGfAmCfGmUfGmGfUmAfGmApsmUp |
| | | | | mCfGmAfCpsmUpsmU | | smU |
| 30 | 932 | 60 | 78 | fCpsmUpsfAmCfCmAfCmGfUmCfGmGfGmGfC | 1514 | mGpsfGpsmUfCmGfCmCfCmCfGmAfCmGfUmGfGmUfAmGpsmUp |
| | | | | mGfAmCfCpsmUpsmU | | smU |
| 31 | 933 | 62 | 80 | fApsmCpsfCmAfCmGfUmCfGmGfGmGfCmGfA | 1515 | mCpsfGpsmGfGmUfCmGfCmCfCmCfGmAfCmGfUmGfGmUpsmUp |
| | | | | mCfCmCfGpsmUpsmU | | smU |
| 32 | 934 | 81 | 99 | fCpsmUpsfGmCfCmUfGmAfGmCfGmAfGmCfA | 1516 | mGpsfGpsmCfGmUfGmCfUmCfGmCfUmCfAmGfGmCfAmGpsmUp |
| | | | | mCfGmCfCpsmUpsmU | | smU |
| 33 | 935 | 82 | 100 | fUpsmGpsfCmCfUmGfAmGfCmGfAmGfCmAfC | 1517 | mGpsfGpsmGfCmGfUmGfCmUfCmGfCmUfCmAfGmGfCmApsmUp |
| | | | | mGfCmCfCpsmUpsmU | | smU |
| 34 | 936 | 84 | 102 | fCpsmCpsfUmGfAmGfCmGfAmGfCmAfCmGfC | 1518 | mCpsfGpsmGfGmGfCmGfUmGfCmUfCmGfCmUfCmAfGmGpsmU |
| | | | | mCfCmCfGpsmUpsmU | | psmU |
| 35 | 937 | 87 | 105 | fGpsmApsfGmCfGmAfGmCfAmCfGmCfCmCfC | 1519 | mGpsfUpsmGfCmGfGmGfGmCfGmUfGmCfUmCfGmCfUmCpsmU |
| | | | | mGfCmAfCpsmUpsmU | | psmU |
| 36 | 938 | 88 | 106 | fApsmGpsfCmGfAmGfCmAfCmGfCmCfCmCfG | 1520 | mGpsfGpsmUfGmCfGmGfGmGfCmGfUmGfCmUfCmGfCmUpsmU |
| | | | | mCfAmCfCpsmUpsmU | | psmU |
| 37 | 939 | 91 | 109 | fGpsmApsfGmCfAmCfGmCfCmCfCmGfCmAfC | 1521 | mGpsfGpsmAfGmGfUmGfCmGfGmGfGmCfGmUfGmCfUmCpsmU |
| | | | | mCfUmCfCpsmUpsmU | | psmU |
| 38 | 940 | 98 | 116 | fCpsmCpsfCmCfGmCfAmCfCmUfCmCfUmCfC | 1522 | mUpsfCpsmGfCmGfGmAfGmGfAmGfGmUfGmCfGmGfGmGpsmU |
| | | | | mGfCmGfApsmUpsmU | | psmU |
| 39 | 941 | 99 | 117 | fCpsmCpsfCmGfCmAfCmCfUmCfCmUfCmCfG | 1523 | mGpsfUpsmCfGmCfGmGfAmGfGmAfGmGfUmGfCmGfGmGpsmU |
| | | | | mCfGmAfCpsmUpsmU | | psmU |
| 40 | 942 | 100 | 118 | fCpsmCpsfGmCfAmCfCmUfCmCfUmCfCmGfC | 1524 | mCpsfGpsmUfCmGfCmGfGmAfGmGfAmGfGmUfGmCfGmGpsmU |
| | | | | mGfAmCfGpsmUpsmU | | psmU |
| 41 | 943 | 101 | 119 | fCpsmGpsfCmAfCmCfUmCfCmUfCmCfGmCfG | 1525 | mGpsfCpsmGfUmCfGmCfGmGfAmGfGmAfGmGfUmGfCmGpsmU |
| | | | | mAfCmGfCpsmUpsmU | | psmU |
| 42 | 944 | 105 | 123 | fCpsmCpsfUmCfCmUfCmCfGmCfGmAfCmGfC | 1526 | mGpsfCpsmGfCmGfCmGfUmCfGmCfGmGfAmGfGmAfGmGpsmU |
| | | | | mGfCmGfCpsmUpsmU | | psmU |
| 43 | 945 | 106 | 124 | fCpsmUpsfCmCfUmCfCmGfCmGfAmCfGmCfG | 1527 | mUpsfGpsmCfGmCfGmCfGmUfCmGfCmGfGmAfGmGfAmGpsmU |
| | | | | mCfGmCfApsmUpsmU | | psmU |
| 44 | 946 | 107 | 125 | fUpsmCpsfCmUfCmCfGmCfGmAfCmGfCmGfC | 1528 | mApsfUpsmGfCmGfCmGfCmGfUmCfGmCfGmGfAmGfGmApsmUp |
| | | | | mGfCmAfUpsmUpsmU | | smU |
| 45 | 947 | 108 | 126 | fCpsmCpsfUmCfCmGfCmGfAmCfGmCfGmCfG | 1529 | mCpsfApsmUfGmCfGmCfGmCfGmUfCmGfCmGfGmAfGmGpsmUp |
| | | | | mCfAmUfGpsmUpsmU | | smU |
| 46 | 948 | 109 | 127 | fCpsmUpsfCmCfGmCfGmAfCmGfCmGfCmGfC | 1530 | mApsfCpsmAfUmGfCmGfCmGfCmGfUmCfGmCfGmGfAmGpsmUp |
| | | | | mAfUmGfUpsmUpsmU | | smU |
| 47 | 949 | 110 | 128 | fUpsmCpsfCmGfCmGfAmCfGmCfGmCfGmCfA | 1531 | mApsfApsmCfAmUfGmCfGmCfGmCfGmUfCmGfCmGfGmApsmUp |
| | | | | mUfGmUfUpsmUpsmU | | smU |
| 48 | 950 | 111 | 129 | fCpsmCpsfGmCfGmAfCmGfCmGfCmGfCmAfU | 1532 | mCpsfApsmAfCmAfUmGfCmGfCmGfCmGfUmCfGmCfGmGpsmUp |
| | | | | mGfUmUfGpsmUpsmU | | smU |
| 49 | 951 | 112 | 130 | fCpsmGpsfCmGfAmCfGmCfGmCfGmCfAmUfG | 1533 | mApsfCpsmAfAmCfAmUfGmCfGmCfGmCfGmUfCmGfCmGpsmUp |
| | | | | mUfUmGfUpsmUpsmU | | smU |
| 50 | 952 | 113 | 131 | fGpsmCpsfGmAfCmGfCmGfCmGfCmAfUmGfU | 1534 | mApsfApsmCfAmAfCmAfUmGfCmGfCmGfCmGfUmCfGmCpsmUp |
| | | | | mUfGmUfUpsmUpsmU | | smU |
| 51 | 953 | 115 | 133 | fGpsmApsfCmGfCmGfCmGfCmAfUmGfUmUf | 1535 | mCpsfGpsmAfAmCfAmAfCmAfUmGfCmGfCmGfCmGfUmCpsmUp |
| | | | | GmUfUmCfGpsmUpsmU | | smU |
| 52 | 954 | 116 | 134 | fApsmCpsfGmCfGmCfGmCfAmUfGmUfUmGf | 1536 | mCpsfCpsmGfAmAfCmAfAmCfAmUfGmCfGmCfGmCfGmUpsmUp |
| | | | | UmUfCmGfGpsmUpsmU | | smU |
| 53 | 955 | 117 | 135 | fCpsmGpsfCmGfCmGfCmAfUmGfUmUfGmUf | 1537 | mGpsfCpsmCfGmAfAmCfAmAfCmAfUmGfCmGfCmGfCmGpsmUp |
| | | | | UmCfGmGfCpsmUpsmU | | smU |
| 54 | 956 | 118 | 136 | fGpsmCpsfGmCfGmCfAmUfGmUfUmGfUmUf | 1538 | mCpsfGpsmCfCmGfAmAfCmAfAmCfAmUfGmCfGmCfGmCpsmUps |
| | | | | CmGfGmCfGpsmUpsmU | | mU |
| 55 | 957 | 127 | 145 | fUpsmUpsfGmUfUmCfGmGfCmGfCmUfUmCf | 1539 | mCpsfGpsmGfCmCfGmAfAmGfCmGfCmCfGmAfAmCfAmApsmUp |
| | | | | GmGfCmCfGpsmUpsmU | | smU |
| 56 | 958 | 128 | 146 | fUpsmGpsfUmUfCmGfGmCfGmCfUmUfCmGf | 1540 | mCpsfCpsmGfGmCfCmGfAmAfGmCfGmCfCmGfAmAfCmApsmUps |
| | | | | GmCfCmGfGpsmUpsmU | | mU |
| 57 | 959 | 139 | 157 | fUpsmCpsfGmGfCmCfGmGfGmGfCmGfUmUf | 1541 | mApsfGpsmUfGmCfAmAfCmGfCmCfCmCfGmGfCmCfGmApsmUp |
| | | | | GmCfAmCfUpsmUpsmU | | smU |
| 58 | 960 | 214 | 232 | fCpsmUpsfUmGfUmGfCmGfGmAfAmGfGmCf | 1542 | mUpsfCpsmCfUmGfGmCfCmUfUmCfCmGfCmAfCmAfAmGpsmUp |
| | | | | CmAfGmGfApsmUpsmU | | smU |
| 59 | 961 | 216 | 234 | fUpsmGpsfUmGfCmGfGmAfAmGfGmCfCmAf | 1543 | mApsfCpsmUfCmCfUmGfGmCfCmUfUmCfCmGfCmAfCmApsmUp |
| | | | | GmGfAmGfUpsmUpsmU | | smU |
| 60 | 962 | 218 | 236 | fUpsmGpsfCmGfGmAfAmGfGmCfCmAfGmGf | 1544 | mCpsfGpsmAfCmUfCmCfUmGfGmCfCmUfUmCfCmGfCmApsmUp |
| | | | | AmGfUmCfGpsmUpsmU | | smU |
| 61 | 963 | 220 | 238 | fCpsmGpsfGmAfAmGfGmCfCmAfGmGfAmGf | 1545 | mUpsfCpsmCfGmAfCmUfCmCfUmGfGmCfCmUfUmCfCmGpsmUp |
| | | | | UmCfGmGfApsmUpsmU | | smU |
| 62 | 964 | 223 | 241 | fApsmApsfGmGfCmCfAmGfGmAfGmUfCmGf | 1546 | mUpsfGpsmUfUmCfCmGfAmCfUmCfCmUfGmGfCmCfUmUpsmUp |
| | | | | GmAfAmCfApsmUpsmU | | smU |
| 63 | 965 | 224 | 242 | fApsmGpsfGmCfCmAfGmGfAmGfUmCfGmGf | 1547 | mApsfUpsmGfUmUfCmCfGmAfCmUfCmCfUmGfGmCfCmUpsmUp |
| | | | | AmAfCmAfUpsmUpsmU | | smU |
| 64 | 966 | 225 | 243 | fGpsmGpsfCmCfAmGfGmAfGmUfCmGfGmAf | 1548 | mApsfApsmUfGmUfUmCfCmGfAmCfUmCfCmUfGmGfCmCpsmUp |
| | | | | AmCfAmUfUpsmUpsmU | | smU |
| 65 | 967 | 228 | 246 | fCpsmApsfGmGfAmGfUmCfGmGfAmAfCmAfU | 1549 | mGpsfCpsmCfAmAfUmGfUmUfCmCfGmAfCmUfCmCfUmGpsmUp |
| | | | | mUfGmGfCpsmUpsmU | | smU |
| 66 | 968 | 229 | 247 | fApsmGpsfGmAfGmUfCmGfGmAfAmCfAmUf | 1550 | mUpsfGpsmCfCmAfAmUfGmUfUmCfCmGfAmCfUmCfCmUpsmUp |
| | | | | UmGfGmCfApsmUpsmU | | smU |
| 67 | 969 | 235 | 253 | fCpsmGpsfGmAfAmCfAmUfUmGfGmCfAmUfC | 1551 | mGpsfGpsmAfAmGfAmUfGmCfCmAfAmUfGmUfUmCfCmGpsmU |
| | | | | mUfUmCfCpsmUpsmU | | psmU |
| 68 | 970 | 238 | 256 | fApsmApsfCmAfUmUfGmGfCmAfUmCfUmUfC | 1552 | mGpsfApsmUfGmGfAmAfGmAfUmGfCmCfAmAfUmGfUmUpsmU |
| | | | | mCfAmUfCpsmUpsmU | | psmU |
| 69 | 971 | 239 | 257 | fApsmCpsfAmUfUmGfGmCfAmUfCmUfUmCfC | 1553 | mGpsfGpsmAfUmGfGmAfAmGfAmUfGmCfCmAfAmUfGmUpsmU |
| | | | | mAfUmCfCpsmUpsmU | | psmU |
| 70 | 972 | 259 | 277 | fUpsmCpsfCmUfUmCfAmAfCmUfUmAfAmGfC | 1554 | mApsfCpsmUfUmGfCmUfUmAfAmGfUmUfGmAfAmGfGmApsmU |
| | | | | mAfAmGfUpsmUpsmU | | psmU |
| 71 | 973 | 261 | 279 | fCpsmUpsfUmCfAmAfCmUfUmAfAmGfCmAfA | 1555 | mGpsfApsmAfCmUfUmGfCmUfUmAfAmGfUmUfGmAfAmGpsmU |
| | | | | mGfUmUfCpsmUpsmU | | psmU |
| 72 | 974 | 302 | 320 | fGpsmCpsfCmUfCmCfCmGfGmCfCmAfAmUfG | 1556 | mUpsfGpsmGfAmCfAmUfUmGfGmCfCmGfGmGfAmGfGmCpsmU |
| | | | | mUfCmCfApsmUpsmU | | psmU |
| 73 | 975 | 303 | 321 | fCpsmCpsfUmCfCmCfGmGfCmCfAmAfUmGfU | 1557 | mGpsfUpsmGfGmAfCmAfUmUfGmGfCmCfGmGfGmAfGmGpsmU |
| | | | | mCfCmAfCpsmUpsmU | | psmU |
| 74 | 976 | 311 | 329 | fCpsmCpsfAmAfUmGfUmCfCmAfCmCfAmGfC | 1558 | mApsfUpsmGfAmGfCmUfGmGfUmGfGmAfCmAfUmUfGmGpsmU |
| | | | | mUfCmAfUpsmUpsmU | | psmU |
| 75 | 977 | 313 | 331 | fApsmApsfUmGfUmCfCmAfCmCfAmGfCmUfC | 1559 | mApsfGpsmAfUmGfAmGfCmUfGmGfUmGfGmAfCmAfUmUpsmU |
| | | | | mAfUmCfUpsmUpsmU | | psmU |
| 76 | 978 | 314 | 332 | fApsmUpsfGmUfCmCfAmCfCmAfGmCfUmCfA | 1560 | mGpsfApsmGfAmUfGmAfGmCfUmGfGmUfGmGfAmCfAmUpsmU |
| | | | | mUfCmUfCpsmUpsmU | | psmU |
| 77 | 979 | 316 | 334 | fGpsmUpsfCmCfAmCfCmAfGmCfUmCfAmUfC | 1561 | mCpsfGpsmGfAmGfAmUfGmAfGmCfUmGfGmUfGmGfAmCpsmU |
| | | | | mUfCmCfGpsmUpsmU | | psmU |
| 78 | 980 | 317 | 335 | fUpsmCpsfCmAfCmCfAmGfCmUfCmAfUmCfU | 1562 | mCpsfCpsmGfGmAfGmAfUmGfAmGfCmUfGmGfUmGfGmApsmU |
| | | | | mCfCmGfGpsmUpsmU | | psmU |
| 79 | 981 | 321 | 339 | fCpsmCpsfAmGfCmUfCmAfUmCfUmCfCmGfG | 1563 | mUpsfUpsmUfGmCfCmGfGmAfGmAfUmGfAmGfCmUfGmGpsmU |
| | | | | mCfAmAfApsmUpsmU | | psmU |
| 80 | 982 | 343 | 361 | fGpsmGpsfCmAfUmCfUmCfUmCfUmUfAmCfC | 1564 | mCpsfUpsmCfUmGfGmUfAmAfGmAfGmAfGmAfUmGfCmCpsmU |
| | | | | mAfGmAfGpsmUpsmU | | psmU |
| 81 | 983 | 351 | 369 | fUpsmCpsfUmUfAmCfCmAfGmAfGmUfGmUf | 1565 | mApsfUpsmCfAmGfAmCfAmCfUmCfUmGfGmUfAmAfGmApsmUp |
| | | | | CmUfGmAfUpsmUpsmU | | smU |
| 82 | 984 | 352 | 370 | fCpsmUpsfUmAfCmCfAmGfAmGfUmGfUmCf | 1566 | mCpsfApsmUfCmAfGmAfCmAfCmUfCmUfGmGfUmAfAmGpsmUp |
| | | | | UmGfAmUfGpsmUpsmU | | smU |
| 83 | 985 | 353 | 371 | fUpsmUpsfAmCfCmAfGmAfGmUfGmUfCmUf | 1567 | mCpsfCpsmAfUmCfAmGfAmCfAmCfUmCfUmGfGmUfAmApsmUp |
| | | | | GmAfUmGfGpsmUpsmU | | smU |
| 84 | 986 | 354 | 372 | fUpsmApsfCmCfAmGfAmGfUmGfUmCfUmGf | 1568 | mCpsfCpsmCfAmUfCmAfGmAfCmAfCmUfCmUfGmGfUmApsmUp |
| | | | | AmUfGmGfGpsmUpsmU | | smU |
| 85 | 987 | 359 | 377 | fGpsmApsfGmUfGmUfCmUfGmAfUmGfGmGf | 1569 | mUpsfUpsmUfUmCfCmCfCmAfUmCfAmGfAmCfAmCfUmCpsmUp |
| | | | | GmAfAmAfApsmUpsmU | | smU |
| 86 | 988 | 361 | 379 | fGpsmUpsfGmUfCmUfGmAfUmGfGmGfGmAf | 1570 | mCpsfGpsmUfUmUfUmCfCmCfCmAfUmCfAmGfAmCfAmCpsmUp |
| | | | | AmAfAmCfGpsmUpsmU | | smU |
| 87 | 989 | 362 | 380 | fUpsmGpsfUmCfUmGfAmUfGmGfGmGfAmAf | 1571 | mApsfCpsmGfUmUfUmUfCmCfCmCfAmUfCmAfGmAfCmApsmUp |
| | | | | AmAfCmGfUpsmUpsmU | | smU |
| 88 | 990 | 363 | 381 | fGpsmUpsfCmUfGmAfUmGfGmGfGmAfAmAf | 1572 | mApsfApsmCfGmUfUmUfUmCfCmCfCmAfUmCfAmGfAmCpsmUp |
| | | | | AmCfGmUfUpsmUpsmU | | smU |
| 89 | 991 | 364 | 382 | fUpsmCpsfUmGfAmUfGmGfGmGfAmAfAmAf | 1573 | mGpsfApsmAfCmGfUmUfUmUfCmCfCmCfAmUfCmAfGmApsmUp |
| | | | | CmGfUmUfCpsmUpsmU | | smU |
| 90 | 992 | 365 | 383 | fCpsmUpsfGmAfUmGfGmGfGmAfAmAfAmCf | 1574 | mApsfGpsmAfAmCfGmUfUmUfUmCfCmCfCmAfUmCfAmGpsmUp |
| | | | | GmUfUmCfUpsmUpsmU | | smU |
| 91 | 993 | 366 | 384 | fUpsmGpsfAmUfGmGfGmGfAmAfAmAfCmGf | 1575 | mCpsfApsmGfAmAfCmGfUmUfUmUfCmCfCmCfAmUfCmApsmUp |
| | | | | UmUfCmUfGpsmUpsmU | | smU |
| 92 | 994 | 367 | 385 | fGpsmApsfUmGfGmGfGmAfAmAfAmCfGmUf | 1576 | mCpsfCpsmAfGmAfAmCfGmUfUmUfUmCfCmCfCmAfUmCpsmUp |
| | | | | UmCfUmGfGpsmUpsmU | | smU |
| 93 | 995 | 369 | 387 | fUpsmGpsfGmGfGmAfAmAfAmCfGmUfUmCf | 1577 | mCpsfApsmCfCmAfGmAfAmCfGmUfUmUfUmCfCmCfCmApsmUp |
| | | | | UmGfGmUfGpsmUpsmU | | smU |
| 94 | 996 | 371 | 389 | fGpsmGpsfGmAfAmAfAmCfGmUfUmCfUmGf | 1578 | mGpsfApsmCfAmCfCmAfGmAfAmCfGmUfUmUfUmCfCmCpsmUp |
| | | | | GmUfGmUfCpsmUpsmU | | smU |
| 95 | 997 | 372 | 390 | fGpsmGpsfAmAfAmAfCmGfUmUfCmUfGmGf | 1579 | mApsfGpsmAfCmAfCmCfAmGfAmAfCmGfUmUfUmUfCmCpsmUp |
| | | | | UmGfUmCfUpsmUpsmU | | smU |
| 96 | 998 | 375 | 393 | fApsmApsfAmCfGmUfUmCfUmGfGmUfGmUf | 1580 | mGpsfUpsmCfAmGfAmCfAmCfCmAfGmAfAmCfGmUfUmUpsmUp |
| | | | | CmUfGmAfCpsmUpsmU | | smU |
| 97 | 999 | 376 | 394 | fApsmApsfCmGfUmUfCmUfGmGfUmGfUmCf | 1581 | mApsfGpsmUfCmAfGmAfCmAfCmCfAmGfAmAfCmGfUmUpsmUp |
| | | | | UmGfAmCfUpsmUpsmU | | smU |
| 98 | 1000 | 377 | 395 | fApsmCpsfGmUfUmCfUmGfGmUfGmUfCmUf | 1582 | mApsfApsmGfUmCfAmGfAmCfAmCfCmAfGmAfAmCfGmUpsmUp |
| | | | | GmAfCmUfUpsmUpsmU | | smU |
| 99 | 1001 | 378 | 396 | fCpsmGpsfUmUfCmUfGmGfUmGfUmCfUmGf | 1583 | mApsfApsmAfGmUfCmAfGmAfCmAfCmCfAmGfAmAfCmGpsmUp |
| | | | | AmCfUmUfUpsmUpsmU | | smU |
| 100 | 1002 | 379 | 397 | fGpsmUpsfUmCfUmGfGmUfGmUfCmUfGmAf | 1584 | mGpsfApsmAfAmGfUmCfAmGfAmCfAmCfCmAfGmAfAmCpsmUp |
| | | | | CmUfUmUfCpsmUpsmU | | smU |
| 101 | 1003 | 380 | 398 | fUpsmUpsfCmUfGmGfUmGfUmCfUmGfAmCf | 1585 | mCpsfGpsmAfAmAfGmUfCmAfGmAfCmAfCmCfAmGfAmApsmUp |
| | | | | UmUfUmCfGpsmUpsmU | | smU |
| 102 | 1004 | 382 | 400 | fCpsmUpsfGmGfUmGfUmCfUmGfAmCfUmUf | 1586 | mApsfCpsmCfGmAfAmAfGmUfCmAfGmAfCmAfCmCfAmGpsmUp |
| | | | | UmCfGmGfUpsmUpsmU | | smU |
| 103 | 1005 | 383 | 401 | fUpsmGpsfGmUfGmUfCmUfGmAfCmUfUmUf | 1587 | mGpsfApsmCfCmGfAmAfAmGfUmCfAmGfAmCfAmCfCmApsmUp |
| | | | | CmGfGmUfCpsmUpsmU | | smU |
| 104 | 1006 | 384 | 402 | fGpsmGpsfUmGfUmCfUmGfAmCfUmUfUmCf | 1588 | mGpsfGpsmAfCmCfGmAfAmAfGmUfCmAfGmAfCmAfCmCpsmUp |
| | | | | GmGfUmCfCpsmUpsmU | | smU |
| 105 | 1007 | 385 | 403 | fGpsmUpsfGmUfCmUfGmAfCmUfUmUfCmGf | 1589 | mUpsfGpsmGfAmCfCmGfAmAfAmGfUmCfAmGfAmCfAmCpsmUp |
| | | | | GmUfCmCfApsmUpsmU | | smU |
| 106 | 1008 | 386 | 404 | fUpsmGpsfUmCfUmGfAmCfUmUfUmCfGmGf | 1590 | mUpsfUpsmGfGmAfCmCfGmAfAmAfGmUfCmAfGmAfCmApsmUp |
| | | | | UmCfCmAfApsmUpsmU | | smU |
| 107 | 1009 | 387 | 405 | fGpsmUpsfCmUfGmAfCmUfUmUfCmGfGmUf | 1591 | mUpsfUpsmUfGmGfAmCfCmGfAmAfAmGfUmCfAmGfAmCpsmUp |
| | | | | CmCfAmAfApsmUpsmU | | smU |
| 108 | 1010 | 398 | 416 | fGpsmGpsfUmCfCmAfAmAfGmAfCmGfAmAfG | 1592 | mApsfCpsmGfAmCfUmUfCmGfUmCfUmUfUmGfGmAfCmCpsmUp |
| | | | | mUfCmGfUpsmUpsmU | | smU |
| 109 | 1011 | 399 | 417 | fGpsmUpsfCmCfAmAfAmGfAmCfGmAfAmGfU | 1593 | mCpsfApsmCfGmAfCmUfUmCfGmUfCmUfUmUfGmGfAmCpsmUp |
| | | | | mCfGmUfGpsmUpsmU | | smU |
| 110 | 1012 | 400 | 418 | fUpsmCpsfCmAfAmAfGmAfCmGfAmAfGmUfC | 1594 | mCpsfCpsmAfCmGfAmCfUmUfCmGfUmCfUmUfUmGfGmApsmUp |
| | | | | mGfUmGfGpsmUpsmU | | smU |
| 111 | 1013 | 401 | 419 | fCpsmCpsfAmAfAmGfAmCfGmAfAmGfUmCfG | 1595 | mUpsfCpsmCfAmCfGmAfCmUfUmCfGmUfCmUfUmUfGmGpsmU |
| | | | | mUfGmGfApsmUpsmU | | psmU |
| 112 | 1014 | 402 | 420 | fCpsmApsfAmAfGmAfCmGfAmAfGmUfCmGfU | 1596 | mApsfUpsmCfCmAfCmGfAmCfUmUfCmGfUmCfUmUfUmGpsmUp |
| | | | | mGfGmAfUpsmUpsmU | | smU |
| 113 | 1015 | 404 | 422 | fApsmApsfGmAfCmGfAmAfGmUfCmGfUmGf | 1597 | mGpsfCpsmAfUmCfCmAfCmGfAmCfUmUfCmGfUmCfUmUpsmUp |
| | | | | GmAfUmGfCpsmUpsmU | | smU |
| 114 | 1016 | 405 | 423 | fApsmGpsfAmCfGmAfAmGfUmCfGmUfGmGf | 1598 | mGpsfGpsmCfAmUfCmCfAmCfGmAfCmUfUmCfGmUfCmUpsmUp |
| | | | | AmUfGmCfCpsmUpsmU | | smU |
| 115 | 1017 | 406 | 424 | fGpsmApsfCmGfAmAfGmUfCmGfUmGfGmAf | 1599 | mApsfGpsmGfCmAfUmCfCmAfCmGfAmCfUmUfCmGfUmCpsmUp |
| | | | | UmGfCmCfUpsmUpsmU | | smU |
| 116 | 1018 | 407 | 425 | fApsmCpsfGmAfAmGfUmCfGmUfGmGfAmUf | 1600 | mApsfApsmGfGmCfAmUfCmCfAmCfGmAfCmUfUmCfGmUpsmUp |
| | | | | GmCfCmUfUpsmUpsmU | | smU |
| 117 | 1019 | 409 | 427 | fGpsmApsfAmGfUmCfGmUfGmGfAmUfGmCf | 1601 | mCpsfCpsmAfAmGfGmCfAmUfCmCfAmCfGmAfCmUfUmCpsmUps |
| | | | | CmUfUmGfGpsmUpsmU | | mU |
| 118 | 1020 | 410 | 428 | fApsmApsfGmUfCmGfUmGfGmAfUmGfCmCf | 1602 | mApsfCpsmCfAmAfGmGfCmAfUmCfCmAfCmGfAmCfUmUpsmUp |
| | | | | UmUfGmGfUpsmUpsmU | | smU |
| 119 | 1021 | 412 | 430 | fGpsmUpsfCmGfUmGfGmAfUmGfCmCfUmUf | 1603 | mApsfUpsmAfCmCfAmAfGmGfCmAfUmCfCmAfCmGfAmCpsmUps |
| | | | | GmGfUmAfUpsmUpsmU | | mU |
| 120 | 1022 | 413 | 431 | fUpsmCpsfGmUfGmGfAmUfGmCfCmUfUmGf | 1604 | mCpsfApsmUfAmCfCmAfAmGfGmCfAmUfCmCfAmCfGmApsmUps |
| | | | | GmUfAmUfGpsmUpsmU | | mU |
| 121 | 1023 | 415 | 433 | fGpsmUpsfGmGfAmUfGmCfCmUfUmGfGmUf | 1605 | mApsfApsmCfAmUfAmCfCmAfAmGfGmCfAmUfCmCfAmCpsmUps |
| | | | | AmUfGmUfUpsmUpsmU | | mU |
| 122 | 1024 | 416 | 434 | fUpsmGpsfGmAfUmGfCmCfUmUfGmGfUmAf | 1606 | mGpsfApsmAfCmAfUmAfCmCfAmAfGmGfCmAfUmCfCmApsmUp |
| | | | | UmGfUmUfCpsmUpsmU | | smU |
| 123 | 1025 | 417 | 435 | fGpsmGpsfAmUfGmCfCmUfUmGfGmUfAmUf | 1607 | mGpsfGpsmAfAmCfAmUfAmCfCmAfAmGfGmCfAmUfCmCpsmUp |
| | | | | GmUfUmCfCpsmUpsmU | | smU |
| 124 | 1026 | 419 | 437 | fApsmUpsfGmCfCmUfUmGfGmUfAmUfGmUf | 1608 | mCpsfApsmGfGmAfAmCfAmUfAmCfCmAfAmGfGmCfAmUpsmUp |
| | | | | UmCfCmUfGpsmUpsmU | | smU |
| 125 | 1027 | 421 | 439 | fGpsmCpsfCmUfUmGfGmUfAmUfGmUfUmCf | 1609 | mApsfGpsmCfAmGfGmAfAmCfAmUfAmCfCmAfAmGfGmCpsmUp |
| | | | | CmUfGmCfUpsmUpsmU | | smU |
| 126 | 1028 | 429 | 447 | fApsmUpsfGmUfUmCfCmUfGmCfUmUfCmAf | 1610 | mGpsfGpsmGfCmAfUmGfAmAfGmCfAmGfGmAfAmCfAmUpsmUp |
| | | | | UmGfCmCfCpsmUpsmU | | smU |
| 127 | 1029 | 432 | 450 | fUpsmUpsfCmCfUmGfCmUfUmCfAmUfGmCfC | 1611 | mGpsfApsmAfGmGfGmCfAmUfGmAfAmGfCmAfGmGfAmApsmU |
| | | | | mCfUmUfCpsmUpsmU | | psmU |
| 128 | 1030 | 433 | 451 | fUpsmCpsfCmUfGmCfUmUfCmAfUmGfCmCfC | 1612 | mApsfGpsmAfAmGfGmGfCmAfUmGfAmAfGmCfAmGfGmApsmU |
| | | | | mUfUmCfUpsmUpsmU | | psmU |
| 129 | 1031 | 456 | 474 | fUpsmGpsfGmCfCmUfUmAfUmCfCmCfUmCfC | 1613 | mGpsfGpsmAfAmGfGmAfGmGfGmAfUmAfAmGfGmCfCmApsmU |
| | | | | mUfUmCfCpsmUpsmU | | psmU |
| 130 | 1032 | 461 | 479 | fUpsmUpsfAmUfCmCfCmUfCmCfUmUfCmCfU | 1614 | mCpsfUpsmGfAmAfGmGfAmAfGmGfAmGfGmGfAmUfAmApsmU |
| | | | | mUfCmAfGpsmUpsmU | | psmU |
| 131 | 1033 | 462 | 480 | fUpsmApsfUmCfCmCfUmCfCmUfUmCfCmUfU | 1615 | mUpsfCpsmUfGmAfAmGfGmAfAmGfGmAfGmGfGmAfUmApsmU |
| | | | | mCfAmGfApsmUpsmU | | psmU |
| 132 | 1034 | 466 | 484 | fCpsmCpsfUmCfCmUfUmCfCmUfUmCfAmGfA | 1616 | mCpsfGpsmCfCmUfCmUfGmAfAmGfGmAfAmGfGmAfGmGpsmUp |
| | | | | mGfGmCfGpsmUpsmU | | smU |
| 133 | 1035 | 467 | 485 | fCpsmUpsfCmCfUmUfCmCfUmUfCmAfGmAfG | 1617 | mApsfCpsmGfCmCfUmCfUmGfAmAfGmGfAmAfGmGfAmGpsmUp |
| | | | | mGfCmGfUpsmUpsmU | | smU |
| 134 | 1036 | 469 | 487 | fCpsmCpsfUmUfCmCfUmUfCmAfGmAfGmGfC | 1618 | mGpsfCpsmAfCmGfCmCfUmCfUmGfAmAfGmGfAmAfGmGpsmUp |
| | | | | mGfUmGfCpsmUpsmU | | smU |
| 135 | 1037 | 470 | 488 | fCpsmUpsfUmCfCmUfUmCfAmGfAmGfGmCfG | 1619 | mCpsfGpsmCfAmCfGmCfCmUfCmUfGmAfAmGfGmAfAmGpsmUp |
| | | | | mUfGmCfGpsmUpsmU | | smU |
| 136 | 1038 | 471 | 489 | fUpsmUpsfCmCfUmUfCmAfGmAfGmGfCmGf | 1620 | mUpsfCpsmGfCmAfCmGfCmCfUmCfUmGfAmAfGmGfAmApsmUp |
| | | | | UmGfCmGfApsmUpsmU | | smU |
| 137 | 1039 | 472 | 490 | fUpsmCpsfCmUfUmCfAmGfAmGfGmCfGmUf | 1621 | mApsfUpsmCfGmCfAmCfGmCfCmUfCmUfGmAfAmGfGmApsmUp |
| | | | | GmCfGmAfUpsmUpsmU | | smU |
| 138 | 1040 | 473 | 491 | fCpsmCpsfUmUfCmAfGmAfGmGfCmGfUmGfC | 1622 | mUpsfApsmUfCmGfCmAfCmGfCmCfUmCfUmGfAmAfGmGpsmUp |
| | | | | mGfAmUfApsmUpsmU | | smU |
| 139 | 1041 | 489 | 507 | fApsmUpsfAmUfGmUfGmGfAmUfGmGfAmGf | 1623 | mCpsfApsmCfUmCfCmUfCmCfAmUfCmCfAmCfAmUfAmUpsmUps |
| | | | | GmAfGmUfGpsmUpsmU | | mU |
| 140 | 1042 | 494 | 512 | fUpsmGpsfGmAfUmGfGmAfGmGfAmGfUmGf | 1624 | mUpsfCpsmAfCmUfCmAfCmUfCmCfUmCfCmAfUmCfCmApsmUps |
| | | | | AmGfUmGfApsmUpsmU | | mU |
| 141 | 1043 | 497 | 515 | fApsmUpsfGmGfAmGfGmAfGmUfGmAfGmUf | 1625 | mUpsfUpsmGfUmCfAmCfUmCfAmCfUmCfCmUfCmCfAmUpsmUp |
| | | | | GmAfCmAfApsmUpsmU | | smU |
| 142 | 1044 | 501 | 519 | fApsmGpsfGmAfGmUfGmAfGmUfGmAfCmAf | 1626 | mUpsfApsmCfGmUfUmGfUmCfAmCfUmCfAmCfUmCfCmUpsmUp |
| | | | | AmCfGmUfApsmUpsmU | | smU |
| 143 | 1045 | 502 | 520 | fGpsmGpsfAmGfUmGfAmGfUmGfAmCfAmAf | 1627 | mGpsfUpsmAfCmGfUmUfGmUfCmAfCmUfCmAfCmUfCmCpsmUp |
| | | | | CmGfUmAfCpsmUpsmU | | smU |
| 144 | 1046 | 504 | 522 | fApsmGpsfUmGfAmGfUmGfAmCfAmAfCmGf | 1628 | mGpsfGpsmGfUmAfCmGfUmUfGmUfCmAfCmUfCmAfCmUpsmU |
| | | | | UmAfCmCfCpsmUpsmU | | psmU |
| 145 | 1047 | 505 | 523 | fGpsmUpsfGmAfGmUfGmAfCmAfAmCfGmUf | 1629 | mApsfGpsmGfGmUfAmCfGmUfUmGfUmCfAmCfUmCfAmCpsmUp |
| | | | | AmCfCmCfUpsmUpsmU | | smU |
| 146 | 1048 | 507 | 525 | fGpsmApsfGmUfGmAfCmAfAmCfGmUfAmCfC | 1630 | mGpsfApsmAfGmGfGmUfAmCfGmUfUmGfUmCfAmCfUmCpsmU |
| | | | | mCfUmUfCpsmUpsmU | | psmU |
| 147 | 1049 | 530 | 548 | fApsmUpsfGmCfCmAfAmAfAmCfAmAfCmCfA | 1631 | mGpsfUpsmGfAmUfGmGfUmUfGmUfUmUfUmGfGmCfAmUpsmU |
| | | | | mUfCmAfCpsmUpsmU | | psmU |
| 148 | 1050 | 531 | 549 | fUpsmGpsfCmCfAmAfAmAfCmAfAmCfCmAfU | 1632 | mGpsfGpsmUfGmAfUmGfGmUfUmGfUmUfUmUfGmGfCmApsmU |
| | | | | mCfAmCfCpsmUpsmU | | psmU |
| 149 | 1051 | 532 | 550 | fGpsmCpsfCmAfAmAfAmCfAmAfCmCfAmUfC | 1633 | mCpsfGpsmGfUmGfAmUfGmGfUmUfGmUfUmUfUmGfGmCpsmU |
| | | | | mAfCmCfGpsmUpsmU | | psmU |
| 150 | 1052 | 535 | 553 | fApsmApsfAmAfCmAfAmCfCmAfUmCfAmCfC | 1634 | mApsfCpsmAfCmGfGmUfGmAfUmGfGmUfUmGfUmUfUmUpsmU |
| | | | | mGfUmGfUpsmUpsmU | | psmU |
| 151 | 1053 | 536 | 554 | fApsmApsfAmCfAmAfCmCfAmUfCmAfCmCfG | 1635 | mGpsfApsmCfAmCfGmGfUmGfAmUfGmGfUmUfGmUfUmUpsmU |
| | | | | mUfGmUfCpsmUpsmU | | psmU |
| 152 | 1054 | 538 | 556 | fApsmCpsfAmAfCmCfAmUfCmAfCmCfGmUfG | 1636 | mGpsfGpsmGfAmCfAmCfGmGfUmGfAmUfGmGfUmUfGmUpsmU |
| | | | | mUfCmCfCpsmUpsmU | | psmU |
| 153 | 1055 | 539 | 557 | fCpsmApsfAmCfCmAfUmCfAmCfCmGfUmGfU | 1637 | mGpsfGpsmGfGmAfCmAfCmGfGmUfGmAfUmGfGmUfUmGpsmU |
| | | | | mCfCmCfCpsmUpsmU | | psmU |
| 154 | 1056 | 540 | 558 | fApsmApsfCmCfAmUfCmAfCmCfGmUfGmUfC | 1638 | mGpsfGpsmGfGmGfAmCfAmCfGmGfUmGfAmUfGmGfUmUpsmU |
| | | | | mCfCmCfCpsmUpsmU | | psmU |
| 155 | 1057 | 546 | 564 | fCpsmApsfCmCfGmUfGmUfCmCfCmCfCmUfU | 1639 | mApsfUpsmAfGmAfAmGfGmGfGmGfAmCfAmCfGmGfUmGpsmU |
| | | | | mCfUmAfUpsmUpsmU | | psmU |
| 156 | 1058 | 547 | 565 | fApsmCpsfCmGfUmGfUmCfCmCfCmCfUmUfC | 1640 | mCpsfApsmUfAmGfAmAfGmGfGmGfGmAfCmAfCmGfGmUpsmU |
| | | | | mUfAmUfGpsmUpsmU | | psmU |
| 157 | 1059 | 550 | 568 | fGpsmUpsfGmUfCmCfCmCfCmUfUmCfUmAfU | 1641 | mCpsfCpsmCfCmAfUmAfGmAfAmGfGmGfGmGfAmCfAmCpsmUp |
| | | | | mGfGmGfGpsmUpsmU | | smU |
| 158 | 1060 | 551 | 569 | fUpsmGpsfUmCfCmCfCmCfUmUfCmUfAmUfG | 1642 | mUpsfCpsmCfCmCfAmUfAmGfAmAfGmGfGmGfGmAfCmApsmUp |
| | | | | mGfGmGfApsmUpsmU | | smU |
| 159 | 1061 | 552 | 570 | fGpsmUpsfCmCfCmCfCmUfUmCfUmAfUmGfG | 1643 | mCpsfUpsmCfCmCfCmAfUmAfGmAfAmGfGmGfGmGfAmCpsmUp |
| | | | | mGfGmAfGpsmUpsmU | | smU |
| 160 | 1062 | 553 | 571 | fUpsmCpsfCmCfCmCfUmUfCmUfAmUfGmGfG | 1644 | mApsfCpsmUfCmCfCmCfAmUfAmGfAmAfGmGfGmGfGmApsmUp |
| | | | | mGfAmGfUpsmUpsmU | | smU |
| 161 | 1063 | 556 | 574 | fCpsmCpsfCmUfUmCfUmAfUmGfGmGfGmAf | 1645 | mCpsfGpsmUfAmCfUmCfCmCfCmAfUmAfGmAfAmGfGmGpsmUp |
| | | | | GmUfAmCfGpsmUpsmU | | smU |
| 162 | 1064 | 557 | 575 | fCpsmCpsfUmUfCmUfAmUfGmGfGmGfAmGf | 1646 | mUpsfCpsmGfUmAfCmUfCmCfCmCfAmUfAmGfAmAfGmGpsmUp |
| | | | | UmAfCmGfApsmUpsmU | | smU |
| 163 | 1065 | 559 | 577 | fUpsmUpsfCmUfAmUfGmGfGmGfAmGfUmAf | 1647 | mUpsfGpsmUfCmGfUmAfCmUfCmCfCmCfAmUfAmGfAmApsmUp |
| | | | | CmGfAmCfApsmUpsmU | | smU |
| 164 | 1066 | 560 | 578 | fUpsmCpsfUmAfUmGfGmGfGmAfGmUfAmCf | 1648 | mApsfUpsmGfUmCfGmUfAmCfUmCfCmCfCmAfUmAfGmApsmUp |
| | | | | GmAfCmAfUpsmUpsmU | | smU |
| 165 | 1067 | 561 | 579 | fCpsmUpsfAmUfGmGfGmGfAmGfUmAfCmGf | 1649 | mGpsfApsmUfGmUfCmGfUmAfCmUfCmCfCmCfAmUfAmGpsmUp |
| | | | | AmCfAmUfCpsmUpsmU | | smU |
| 166 | 1068 | 562 | 580 | fUpsmApsfUmGfGmGfGmAfGmUfAmCfGmAf | 1650 | mApsfGpsmAfUmGfUmCfGmUfAmCfUmCfCmCfCmAfUmApsmUp |
| | | | | CmAfUmCfUpsmUpsmU | | smU |
| 167 | 1069 | 571 | 589 | fUpsmApsfCmGfAmCfAmUfCmUfGmCfCmCfU | 1651 | mCpsfUpsmUfUmAfGmGfGmCfAmGfAmUfGmUfCmGfUmApsmU |
| | | | | mAfAmAfGpsmUpsmU | | psmU |
| 168 | 1070 | 572 | 590 | fApsmCpsfGmAfCmAfUmCfUmGfCmCfCmUfA | 1652 | mApsfCpsmUfUmUfAmGfGmGfCmAfGmAfUmGfUmCfGmUpsmU |
| | | | | mAfAmGfUpsmUpsmU | | psmU |
| 169 | 1071 | 573 | 591 | fCpsmGpsfAmCfAmUfCmUfGmCfCmCfUmAfA | 1653 | mGpsfApsmCfUmUfUmAfGmGfGmCfAmGfAmUfGmUfCmGpsmU |
| | | | | mAfGmUfCpsmUpsmU | | psmU |
| 170 | 1072 | 574 | 592 | fGpsmApsfCmAfUmCfUmGfCmCfCmUfAmAfA | 1654 | mUpsfGpsmAfCmUfUmUfAmGfGmGfCmAfGmAfUmGfUmCpsmU |
| | | | | mGfUmCfApsmUpsmU | | psmU |
| 171 | 1073 | 575 | 593 | fApsmCpsfAmUfCmUfGmCfCmCfUmAfAmAfG | 1655 | mUpsfUpsmGfAmCfUmUfUmAfGmGfGmCfAmGfAmUfGmUpsmU |
| | | | | mUfCmAfApsmUpsmU | | psmU |
| 172 | 1074 | 576 | 594 | fCpsmApsfUmCfUmGfCmCfCmUfAmAfAmGfU | 1656 | mCpsfUpsmUfGmAfCmUfUmUfAmGfGmGfCmAfGmAfUmGpsmU |
| | | | | mCfAmAfGpsmUpsmU | | psmU |
| 173 | 1075 | 577 | 595 | fApsmUpsfCmUfGmCfCmCfUmAfAmAfGmUfC | 1657 | mApsfCpsmUfUmGfAmCfUmUfUmAfGmGfGmCfAmGfAmUpsmU |
| | | | | mAfAmGfUpsmUpsmU | | psmU |
| 174 | 1076 | 582 | 600 | fCpsmCpsfCmUfAmAfAmGfUmCfAmAfGmUfC | 1658 | mCpsfGpsmUfGmGfAmCfUmUfGmAfCmUfUmUfAmGfGmGpsmU |
| | | | | mCfAmCfGpsmUpsmU | | psmU |
| 175 | 1077 | 583 | 601 | fCpsmCpsfUmAfAmAfGmUfCmAfAmGfUmCfC | 1659 | mUpsfCpsmGfUmGfGmAfCmUfUmGfAmCfUmUfUmAfGmGpsmU |
| | | | | mAfCmGfApsmUpsmU | | psmU |
| 176 | 1078 | 585 | 603 | fUpsmApsfAmAfGmUfCmAfAmGfUmCfCmAfC | 1660 | mGpsfUpsmUfCmGfUmGfGmAfCmUfUmGfAmCfUmUfUmApsmU |
| | | | | mGfAmAfCpsmUpsmU | | psmU |
| 177 | 1079 | 586 | 604 | fApsmApsfAmGfUmCfAmAfGmUfCmCfAmCfG | 1661 | mApsfGpsmUfUmCfGmUfGmGfAmCfUmUfGmAfCmUfUmUpsmU |
| | | | | mAfAmCfUpsmUpsmU | | psmU |
| 178 | 1080 | 587 | 605 | fApsmApsfGmUfCmAfAmGfUmCfCmAfCmGfA | 1662 | mApsfApsmGfUmUfCmGfUmGfGmAfCmUfUmGfAmCfUmUpsmU |
| | | | | mAfCmUfUpsmUpsmU | | psmU |
| 179 | 1081 | 588 | 606 | fApsmGpsfUmCfAmAfGmUfCmCfAmCfGmAfA | 1663 | mApsfApsmAfGmUfUmCfGmUfGmGfAmCfUmUfGmAfCmUpsmU |
| | | | | mCfUmUfUpsmUpsmU | | psmU |
| 180 | 1082 | 589 | 607 | fGpsmUpsfCmAfAmGfUmCfCmAfCmGfAmAfC | 1664 | mGpsfApsmAfAmGfUmUfCmGfUmGfGmAfCmUfUmGfAmCpsmU |
| | | | | mUfUmUfCpsmUpsmU | | psmU |
| 181 | 1083 | 590 | 608 | fUpsmCpsfAmAfGmUfCmCfAmCfGmAfAmCfU | 1665 | mApsfGpsmAfAmAfGmUfUmCfGmUfGmGfAmCfUmUfGmApsmU |
| | | | | mUfUmCfUpsmUpsmU | | psmU |
| 182 | 1084 | 591 | 609 | fCpsmApsfAmGfUmCfCmAfCmGfAmAfCmUfU | 1666 | mApsfApsmGfAmAfAmGfUmUfCmGfUmGfGmAfCmUfUmGpsmU |
| | | | | mUfCmUfUpsmUpsmU | | psmU |
| 183 | 1085 | 592 | 610 | fApsmApsfGmUfCmCfAmCfGmAfAmCfUmUfU | 1667 | mGpsfApsmAfGmAfAmAfGmUfUmCfGmUfGmGfAmCfUmUpsmU |
| | | | | mCfUmUfCpsmUpsmU | | psmU |
| 184 | 1086 | 593 | 611 | fApsmGpsfUmCfCmAfCmGfAmAfCmUfUmUfC | 1668 | mUpsfGpsmAfAmGfAmAfAmGfUmUfCmGfUmGfGmAfCmUpsmU |
| | | | | mUfUmCfApsmUpsmU | | psmU |
| 185 | 1087 | 594 | 612 | fGpsmUpsfCmCfAmCfGmAfAmCfUmUfUmCfU | 1669 | mApsfUpsmGfAmAfGmAfAmAfGmUfUmCfGmUfGmGfAmCpsmU |
| | | | | mUfCmAfUpsmUpsmU | | psmU |
| 186 | 1088 | 595 | 613 | fUpsmCpsfCmAfCmGfAmAfCmUfUmUfCmUfU | 1670 | mCpsfApsmUfGmAfAmGfAmAfAmGfUmUfCmGfUmGfGmApsmU |
| | | | | mCfAmUfGpsmUpsmU | | psmU |
| 187 | 1089 | 597 | 615 | fCpsmApsfCmGfAmAfCmUfUmUfCmUfUmCfA | 1671 | mCpsfApsmCfAmUfGmAfAmGfAmAfAmGfUmUfCmGfUmGpsmU |
| | | | | mUfGmUfGpsmUpsmU | | psmU |
| 188 | 1090 | 620 | 638 | fUpsmCpsfAmCfCmAfAmGfCmUfCmAfGmUfC | 1672 | mCpsfGpsmUfAmGfAmCfUmGfAmGfCmUfUmGfGmUfGmApsmU |
| | | | | mUfAmCfGpsmUpsmU | | psmU |
| 189 | 1091 | 621 | 639 | fCpsmApsfCmCfAmAfGmCfUmCfAmGfUmCfU | 1673 | mGpsfCpsmGfUmAfGmAfCmUfGmAfGmCfUmUfGmGfUmGpsmU |
| | | | | mAfCmGfCpsmUpsmU | | psmU |
| 190 | 1092 | 622 | 640 | fApsmCpsfCmAfAmGfCmUfCmAfGmUfCmUfA | 1674 | mGpsfGpsmCfGmUfAmGfAmCfUmGfAmGfCmUfUmGfGmUpsmU |
| | | | | mCfGmCfCpsmUpsmU | | psmU |
| 191 | 1093 | 623 | 641 | fCpsmCpsfAmAfGmCfUmCfAmGfUmCfUmAfC | 1675 | mApsfGpsmGfCmGfUmAfGmAfCmUfGmAfGmCfUmUfGmGpsmU |
| | | | | mGfCmCfUpsmUpsmU | | psmU |
| 192 | 1094 | 624 | 642 | fCpsmApsfAmGfCmUfCmAfGmUfCmUfAmCfG | 1676 | mGpsfApsmGfGmCfGmUfAmGfAmCfUmGfAmGfCmUfUmGpsmU |
| | | | | mCfCmUfCpsmUpsmU | | psmU |
| 193 | 1095 | 626 | 644 | fApsmGpsfCmUfCmAfGmUfCmUfAmCfGmCfC | 1677 | mCpsfApsmGfAmGfGmCfGmUfAmGfAmCfUmGfAmGfCmUpsmUp |
| | | | | mUfCmUfGpsmUpsmU | | smU |
| 194 | 1096 | 630 | 648 | fCpsmApsfGmUfCmUfAmCfGmCfCmUfCmUfG | 1678 | mUpsfGpsmUfGmCfAmGfAmGfGmCfGmUfAmGfAmCfUmGpsmU |
| | | | | mCfAmCfApsmUpsmU | | psmU |
| 195 | 1097 | 631 | 649 | fApsmGpsfUmCfUmAfCmGfCmCfUmCfUmGfC | 1679 | mCpsfUpsmGfUmGfCmAfGmAfGmGfCmGfUmAfGmAfCmUpsmU |
| | | | | mAfCmAfGpsmUpsmU | | psmU |
| 196 | 1098 | 636 | 654 | fApsmCpsfGmCfCmUfCmUfGmCfAmCfAmGfG | 1680 | mGpsfUpsmUfCmCfCmUfGmUfGmCfAmGfAmGfGmCfGmUpsmU |
| | | | | mGfAmAfCpsmUpsmU | | psmU |
| 197 | 1099 | 642 | 660 | fCpsmUpsfGmCfAmCfAmGfGmGfAmAfCmCfU | 1681 | mGpsfUpsmAfGmAfGmGfUmUfCmCfCmUfGmUfGmCfAmGpsmU |
| | | | | mCfUmAfCpsmUpsmU | | psmU |
| 198 | 1100 | 643 | 661 | fUpsmGpsfCmAfCmAfGmGfGmAfAmCfCmUfC | 1682 | mGpsfGpsmUfAmGfAmGfGmUfUmCfCmCfUmGfUmGfCmApsmU |
| | | | | mUfAmCfCpsmUpsmU | | psmU |
| 199 | 1101 | 646 | 664 | fApsmCpsfAmGfGmGfAmAfCmCfUmCfUmAfC | 1683 | mGpsfApsmAfGmGfUmAfGmAfGmGfUmUfCmCfCmUfGmUpsmU |
| | | | | mCfUmUfCpsmUpsmU | | psmU |
| 200 | 1102 | 691 | 709 | fCpsmUpsfCmAfAmGfGmUfGmCfUmGfGmGf | 1684 | mUpsfCpsmUfCmUfCmCfCmAfGmCfAmCfCmUfUmGfAmGpsmUp |
| | | | | AmGfAmGfApsmUpsmU | | smU |
| 201 | 1103 | 694 | 712 | fApsmApsfGmGfUmGfCmUfGmGfGmAfGmAf | 1685 | mApsfUpsmAfUmCfUmCfUmCfCmCfAmGfCmAfCmCfUmUpsmUp |
| | | | | GmAfUmAfUpsmUpsmU | | smU |
| 202 | 1104 | 695 | 713 | fApsmGpsfGmUfGmCfUmGfGmGfAmGfAmGf | 1686 | mCpsfApsmUfAmUfCmUfCmUfCmCfCmAfGmCfAmCfCmUpsmUps |
| | | | | AmUfAmUfGpsmUpsmU | | mU |
| 203 | 1105 | 696 | 714 | fGpsmGpsfUmGfCmUfGmGfGmAfGmAfGmAf | 1687 | mGpsfCpsmAfUmAfUmCfUmCfUmCfCmCfAmGfCmAfCmCpsmUps |
| | | | | UmAfUmGfCpsmUpsmU | | mU |
| 204 | 1106 | 697 | 715 | fGpsmUpsfGmCfUmGfGmGfAmGfAmGfAmUf | 1688 | mGpsfGpsmCfAmUfAmUfCmUfCmUfCmCfCmAfGmCfAmCpsmUp |
| | | | | AmUfGmCfCpsmUpsmU | | smU |
| 205 | 1107 | 698 | 716 | fUpsmGpsfCmUfGmGfGmAfGmAfGmAfUmAf | 1689 | mApsfGpsmGfCmAfUmAfUmCfUmCfUmCfCmCfAmGfCmApsmUp |
| | | | | UmGfCmCfUpsmUpsmU | | smU |
| 206 | 1108 | 703 | 721 | fGpsmGpsfAmGfAmGfAmUfAmUfGmCfCmUf | 1690 | mCpsfUpsmCfGmAfAmGfGmCfAmUfAmUfCmUfCmUfCmCpsmUp |
| | | | | UmCfGmAfGpsmUpsmU | | smU |
| 207 | 1109 | 735 | 753 | fApsmUpsfUmCfAmGfGmUfUmCfUmUfGmGf | 1691 | mCpsfUpsmCfUmUfCmCfAmAfGmAfAmCfCmUfGmAfAmUpsmUp |
| | | | | AmAfGmAfGpsmUpsmU | | smU |
| 208 | 1110 | 737 | 755 | fUpsmCpsfAmGfGmUfUmCfUmUfGmGfAmAf | 1692 | mUpsfUpsmCfUmCfUmUfCmCfAmAfGmAfAmCfCmUfGmApsmUp |
| | | | | GmAfGmAfApsmUpsmU | | smU |
| 209 | 1111 | 743 | 761 | fUpsmCpsfUmUfGmGfAmAfGmAfGmAfAmGf | 1693 | mApsfUpsmGfCmCfCmUfUmCfUmCfUmUfCmCfAmAfGmApsmUp |
| | | | | GmGfCmAfUpsmUpsmU | | smU |
| 210 | 1112 | 744 | 762 | fCpsmUpsfUmGfGmAfAmGfAmGfAmAfGmGf | 1694 | mGpsfApsmUfGmCfCmCfUmUfCmUfCmUfUmCfCmAfAmGpsmUp |
| | | | | GmCfAmUfCpsmUpsmU | | smU |
| 211 | 1113 | 745 | 763 | fUpsmUpsfGmGfAmAfGmAfGmAfAmGfGmGf | 1695 | mApsfGpsmAfUmGfCmCfCmUfUmCfUmCfUmUfCmCfAmApsmUp |
| | | | | CmAfUmCfUpsmUpsmU | | smU |
| 212 | 1114 | 747 | 765 | fGpsmGpsfAmAfGmAfGmAfAmGfGmGfCmAf | 1696 | mGpsfCpsmAfGmAfUmGfCmCfCmUfUmCfUmCfUmUfCmCpsmUp |
| | | | | UmCfUmGfCpsmUpsmU | | smU |
| 213 | 1115 | 748 | 766 | fGpsmApsfAmGfAmGfAmAfGmGfGmCfAmUf | 1697 | mUpsfGpsmCfAmGfAmUfGmCfCmCfUmUfCmUfCmUfUmCpsmUp |
| | | | | CmUfGmCfApsmUpsmU | | smU |
| 214 | 1116 | 749 | 767 | fApsmApsfGmAfGmAfAmGfGmGfCmAfUmCf | 1698 | mUpsfUpsmGfCmAfGmAfUmGfCmCfCmUfUmCfUmCfUmUpsmU |
| | | | | UmGfCmAfApsmUpsmU | | psmU |
| 215 | 1117 | 752 | 770 | fApsmGpsfAmAfGmGfGmCfAmUfCmUfGmCfA | 1699 | mCpsfUpsmGfUmUfGmCfAmGfAmUfGmCfCmCfUmUfCmUpsmU |
| | | | | mAfCmAfGpsmUpsmU | | psmU |
| 216 | 1118 | 755 | 773 | fApsmGpsfGmGfCmAfUmCfUmGfCmAfAmCfA | 1700 | mGpsfGpsmCfCmUfGmUfUmGfCmAfGmAfUmGfCmCfCmUpsmUp |
| | | | | mGfGmCfCpsmUpsmU | | smU |
| 217 | 1119 | 758 | 776 | fGpsmCpsfAmUfCmUfGmCfAmAfCmAfGmGfC | 1701 | mUpsfGpsmGfGmGfCmCfUmGfUmUfGmCfAmGfAmUfGmCpsmU |
| | | | | mCfCmCfApsmUpsmU | | psmU |
| 218 | 1120 | 977 | 995 | fCpsmApsfGmCfAmCfUmGfAmGfUmGfAmAfG | 1702 | mApsfUpsmUfUmCfUmUfCmAfCmUfCmAfGmUfGmCfUmGpsmU |
| | | | | mAfAmAfUpsmUpsmU | | psmU |
| 219 | 1121 | 1026 | 1044 | fUpsmUpsfGmCfAmAfCmUfUmGfCmUfAmCfC | 1703 | mApsfApsmUfGmGfGmUfAmGfCmAfAmGfUmUfGmCfAmApsmU |
| | | | | mCfAmUfUpsmUpsmU | | psmU |
| 220 | 1122 | 1035 | 1053 | fGpsmCpsfUmAfCmCfCmAfUmUfAmGfGmAfU | 1704 | mCpsfApsmUfUmAfUmCfCmUfAmAfUmGfGmGfUmAfGmCpsmU |
| | | | | mAfAmUfGpsmUpsmU | | psmU |
| 221 | 1123 | 1038 | 1056 | fApsmCpsfCmCfAmUfUmAfGmGfAmUfAmAfU | 1705 | mApsfGpsmAfCmAfUmUfAmUfCmCfUmAfAmUfGmGfGmUpsmU |
| | | | | mGfUmCfUpsmUpsmU | | psmU |
| 222 | 1124 | 1039 | 1057 | fCpsmCpsfCmAfUmUfAmGfGmAfUmAfAmUfG | 1706 | mApsfApsmGfAmCfAmUfUmAfUmCfCmUfAmAfUmGfGmGpsmU |
| | | | | mUfCmUfUpsmUpsmU | | psmU |
| 223 | 1125 | 1041 | 1059 | fCpsmApsfUmUfAmGfGmAfUmAfAmUfGmUf | 1707 | mApsfUpsmAfAmGfAmCfAmUfUmAfUmCfCmUfAmAfUmGpsmUp |
| | | | | CmUfUmAfUpsmUpsmU | | smU |
| 224 | 1126 | 1064 | 1082 | fUpsmGpsfCmUfGmCfCmCfUmGfUmAfCmCfC | 1708 | mGpsfGpsmCfAmGfGmGfUmAfCmAfGmGfGmCfAmGfCmApsmUp |
| | | | | mUfGmCfCpsmUpsmU | | smU |
| 225 | 1127 | 1068 | 1086 | fGpsmCpsfCmCfUmGfUmAfCmCfCmUfGmCfC | 1709 | mCpsfApsmCfAmGfGmCfAmGfGmGfUmAfCmAfGmGfGmCpsmUp |
| | | | | mUfGmUfGpsmUpsmU | | smU |
| 226 | 1128 | 1077 | 1095 | fCpsmCpsfUmGfCmCfUmGfUmGfGmAfAmUfC | 1710 | mGpsfGpsmCfAmGfAmUfUmCfCmAfCmAfGmGfCmAfGmGpsmUp |
| | | | | mUfGmCfCpsmUpsmU | | smU |
| 227 | 1129 | 1080 | 1098 | fGpsmCpsfCmUfGmUfGmGfAmAfUmCfUmGf | 1711 | mApsfApsmUfGmGfCmAfGmAfUmUfCmCfAmCfAmGfGmCpsmUp |
| | | | | CmCfAmUfUpsmUpsmU | | smU |
| 228 | 1130 | 1111 | 1129 | fApsmGpsfAmCfUmGfGmUfGmAfCmAfUmGf | 1712 | mGpsfApsmAfGmCfCmAfUmGfUmCfAmCfCmAfGmUfCmUpsmUp |
| | | | | GmCfUmUfCpsmUpsmU | | smU |
| 229 | 1131 | 1196 | 1214 | fUpsmGpsfUmGfUmCfUmGfCmUfCmCfCmCfG | 1713 | mGpsfApsmGfGmCfGmGfGmGfAmGfCmAfGmAfCmAfCmApsmUp |
| | | | | mCfCmUfCpsmUpsmU | | smU |
| 230 | 1132 | 1198 | 1216 | fUpsmGpsfUmCfUmGfCmUfCmCfCmCfGmCfC | 1714 | mUpsfGpsmGfAmGfGmCfGmGfGmGfAmGfCmAfGmAfCmApsmU |
| | | | | mUfCmCfApsmUpsmU | | psmU |
| 231 | 1133 | 1 | 21 | fApsmUpsfGmUfAmCfGmAfCmGfCmAfGmAfG | 1715 | vmGpsfCpsmCfGmCfGmCfUmCfUmGfCmGfUmCfGmUfAmCfAmU |
| | | | | mCfGmCfGmGpsfC | | psmUpsmU |
| 232 | 1134 | 3 | 23 | fGpsmUpsfAmCfGmAfCmGfCmAfGmAfGmCfG | 1716 | vmCpsfApsmGfCmCfGmCfGmCfUmCfUmGfCmGfUmCfGmUfAmC |
| | | | | mCfGmGfCmUpsfG | | psmUpsmU |
| 233 | 1135 | 4 | 24 | fUpsmApsfCmGfAmCfGmCfAmGfAmGfCmGfC | 1717 | vmCpsfCpsmAfGmCfCmGfCmGfCmUfCmUfGmCfGmUfCmGfUmA |
| | | | | mGfGmCfUmGpsfG | | psmUpsmU |
| 234 | 1136 | 5 | 25 | fApsmCpsfGmAfCmGfCmAfGmAfGmCfGmCfG | 1718 | vmUpsfCpsmCfAmGfCmCfGmCfGmCfUmCfUmGfCmGfUmCfGmU |
| | | | | mGfCmUfGmGpsfA | | psmUpsmU |
| 235 | 1137 | 8 | 28 | fApsmCpsfGmCfAmGfAmGfCmGfCmGfGmCfU | 1719 | vmApsfGpsmCfUmCfCmAfGmCfCmGfCmGfCmUfCmUfGmCfGmU |
| | | | | mGfGmAfGmCpsfU | | psmUpsmU |
| 236 | 1138 | 9 | 29 | fCpsmGpsfCmAfGmAfGmCfGmCfGmGfCmUfG | 1720 | vmApsfApsmGfCmUfCmCfAmGfCmCfGmCfGmCfUmCfUmGfCmG |
| | | | | mGfAmGfCmUpsfU | | psmUpsmU |
| 237 | 1139 | 10 | 30 | fGpsmCpsfAmGfAmGfCmGfCmGfGmCfUmGf | 1721 | vmCpsfApsmAfGmCfUmCfCmAfGmCfCmGfCmGfCmUfCmUfGmCp |
| | | | | GmAfGmCfUmUpsfG | | smUpsmU |
| 238 | 1140 | 11 | 31 | fCpsmApsfGmAfGmCfGmCfGmGfCmUfGmGfA | 1722 | vmApsfCpsmAfAmGfCmUfCmCfAmGfCmCfGmCfGmCfUmCfUmG |
| | | | | mGfCmUfUmGpsfU | | psmUpsmU |
| 239 | 1141 | 12 | 32 | fApsmGpsfAmGfCmGfCmGfGmCfUmGfGmAf | 1723 | vmGpsfApsmCfAmAfGmCfUmCfCmAfGmCfCmGfCmGfCmUfCmU |
| | | | | GmCfUmUfGmUpsfC | | psmUpsmU |
| 240 | 1142 | 13 | 33 | fGpsmApsfGmCfGmCfGmGfCmUfGmGfAmGf | 1724 | vmGpsfGpsmAfCmAfAmGfCmUfCmCfAmGfCmCfGmCfGmCfUmC |
| | | | | CmUfUmGfUmCpsfC | | psmUpsmU |
| 241 | 1143 | 15 | 35 | fGpsmCpsfGmCfGmGfCmUfGmGfAmGfCmUf | 1725 | vmApsfApsmGfGmAfCmAfAmGfCmUfCmCfAmGfCmCfGmCfGmCp |
| | | | | UmGfUmCfCmUpsfU | | smUpsmU |
| 242 | 1144 | 17 | 37 | fGpsmCpsfGmGfCmUfGmGfAmGfCmUfUmGf | 1726 | vmCpsfGpsmAfAmGfGmAfCmAfAmGfCmUfCmCfAmGfCmCfGmCp |
| | | | | UmCfCmUfUmCpsfG | | smUpsmU |
| 243 | 1145 | 20 | 40 | fGpsmCpsfUmGfGmAfGmCfUmUfGmUfCmCf | 1727 | vmCpsfCpsmGfCmGfAmAfGmGfAmCfAmAfGmCfUmCfCmAfGmCp |
| | | | | UmUfCmGfCmGpsfG | | smUpsmU |
| 244 | 1146 | 22 | 42 | fUpsmGpsfGmAfGmCfUmUfGmUfCmCfUmUf | 1728 | vmGpsfCpsmCfCmGfCmGfAmAfGmGfAmCfAmAfGmCfUmCfCmAp |
| | | | | CmGfCmGfGmGpsfC | | smUpsmU |
| 245 | 1147 | 23 | 43 | fGpsmGpsfAmGfCmUfUmGfUmCfCmUfUmCf | 1729 | vmApsfGpsmCfCmCfGmCfGmAfAmGfGmAfCmAfAmGfCmUfCmCp |
| | | | | GmCfGmGfGmCpsfU | | smUpsmU |
| 246 | 1148 | 24 | 44 | fGpsmApsfGmCfUmUfGmUfCmCfUmUfCmGf | 1730 | vmCpsfApsmGfCmCfCmGfCmGfAmAfGmGfAmCfAmAfGmCfUmCp |
| | | | | CmGfGmGfCmUpsfG | | smUpsmU |
| 247 | 1149 | 25 | 45 | fApsmGpsfCmUfUmGfUmCfCmUfUmCfGmCf | 1731 | vmGpsfCpsmAfGmCfCmCfGmCfGmAfAmGfGmAfCmAfAmGfCmU |
| | | | | GmGfGmCfUmGpsfC | | psmUpsmU |
| 248 | 1150 | 28 | 48 | fUpsmUpsfGmUfCmCfUmUfCmGfCmGfGmGf | 1732 | vmGpsfCpsmCfGmCfAmGfCmCfCmGfCmGfAmAfGmGfAmCfAmAp |
| | | | | CmUfGmCfGmGpsfC | | smUpsmU |
| 249 | 1151 | 29 | 49 | fUpsmGpsfUmCfCmUfUmCfGmCfGmGfGmCf | 1733 | vmApsfGpsmCfCmGfCmAfGmCfCmCfGmCfGmAfAmGfGmAfCmAp |
| | | | | UmGfCmGfGmCpsfU | | smUpsmU |
| 250 | 1152 | 30 | 50 | fGpsmUpsfCmCfUmUfCmGfCmGfGmGfCmUf | 1734 | vmApsfApsmGfCmCfGmCfAmGfCmCfCmGfCmGfAmAfGmGfAmCp |
| | | | | GmCfGmGfCmUpsfU | | smUpsmU |
| 251 | 1153 | 40 | 60 | fGpsmGpsfCmUfGmCfGmGfCmUfUmCfCmUf | 1735 | vmGpsfApsmAfGmCfCmCfAmGfGmAfAmGfCmCfGmCfAmGfCmCp |
| | | | | GmGfGmCfUmUpsfC | | smUpsmU |
| 252 | 1154 | 41 | 61 | fGpsmCpsfUmGfCmGfGmCfUmUfCmCfUmGf | 1736 | vmApsfGpsmAfAmGfCmCfCmAfGmGfAmAfGmCfCmGfCmAfGmC |
| | | | | GmGfCmUfUmCpsfU | | psmUpsmU |
| 253 | 1155 | 42 | 62 | fCpsmUpsfGmCfGmGfCmUfUmCfCmUfGmGf | 1737 | vmUpsfApsmGfAmAfGmCfCmCfAmGfGmAfAmGfCmCfGmCfAmG |
| | | | | GmCfUmUfCmUpsfA | | psmUpsmU |
| 254 | 1156 | 43 | 63 | fUpsmGpsfCmGfGmCfUmUfCmCfUmGfGmGf | 1738 | vmGpsfUpsmAfGmAfAmGfCmCfCmAfGmGfAmAfGmCfCmGfCmA |
| | | | | CmUfUmCfUmApsfC | | psmUpsmU |
| 255 | 1157 | 44 | 64 | fGpsmCpsfGmGfCmUfUmCfCmUfGmGfGmCf | 1739 | vmGpsfGpsmUfAmGfAmAfGmCfCmCfAmGfGmAfAmGfCmCfGmC |
| | | | | UmUfCmUfAmCpsfC | | psmUpsmU |
| 256 | 1158 | 48 | 68 | fCpsmUpsfUmCfCmUfGmGfGmCfUmUfCmUf | 1740 | vmApsfCpsmGfUmGfGmUfAmGfAmAfGmCfCmCfAmGfGmAfAmG |
| | | | | AmCfCmAfCmGpsfU | | psmUpsmU |
| 257 | 1159 | 52 | 72 | fCpsmUpsfGmGfGmCfUmUfCmUfAmCfCmAfC | 1741 | vmCpsfCpsmCfGmAfCmGfUmGfGmUfAmGfAmAfGmCfCmCfAmG |
| | | | | mGfUmCfGmGpsfG | | psmUpsmU |
| 258 | 1160 | 53 | 73 | fUpsmGpsfGmGfCmUfUmCfUmAfCmCfAmCfG | 1742 | vmCpsfCpsmCfCmGfAmCfGmUfGmGfUmAfGmAfAmGfCmCfCmA |
| | | | | mUfCmGfGmGpsfG | | psmUpsmU |
| 259 | 1161 | 55 | 75 | fGpsmGpsfCmUfUmCfUmAfCmCfAmCfGmUfC | 1743 | vmCpsfGpsmCfCmCfCmGfAmCfGmUfGmGfUmAfGmAfAmGfCmC |
| | | | | mGfGmGfGmCpsfG | | psmUpsmU |
| 260 | 1162 | 56 | 76 | fGpsmCpsfUmUfCmUfAmCfCmAfCmGfUmCfG | 1744 | vmUpsfCpsmGfCmCfCmCfGmAfCmGfUmGfGmUfAmGfAmAfGmC |
| | | | | mGfGmGfCmGpsfA | | psmUpsmU |
| 261 | 1163 | 57 | 77 | fCpsmUpsfUmCfUmAfCmCfAmCfGmUfCmGfG | 1745 | vmGpsfUpsmCfGmCfCmCfCmGfAmCfGmUfGmGfUmAfGmAfAmG |
| | | | | mGfGmCfGmApsfC | | psmUpsmU |
| 262 | 1164 | 58 | 78 | fUpsmUpsfCmUfAmCfCmAfCmGfUmCfGmGfG | 1746 | vmGpsfGpsmUfCmGfCmCfCmCfGmAfCmGfUmGfGmUfAmGfAmA |
| | | | | mGfCmGfAmCpsfC | | psmUpsmU |
| 263 | 1165 | 60 | 80 | fCpsmUpsfAmCfCmAfCmGfUmCfGmGfGmGfC | 1747 | vmCpsfGpsmGfGmUfCmGfCmCfCmCfGmAfCmGfUmGfGmUfAmG |
| | | | | mGfAmCfCmCpsfG | | psmUpsmU |
| 264 | 1166 | 82 | 102 | fUpsmGpsfCmCfUmGfAmGfCmGfAmGfCmAfC | 1748 | vmCpsfGpsmGfGmGfCmGfUmGfCmUfCmGfCmUfCmAfGmGfCmA |
| | | | | mGfCmCfCmCpsfG | | psmUpsmU |
| 265 | 1167 | 83 | 103 | fGpsmCpsfCmUfGmAfGmCfGmAfGmCfAmCfG | 1749 | vmGpsfCpsmGfGmGfGmCfGmUfGmCfUmCfGmCfUmCfAmGfGmC |
| | | | | mCfCmCfCmGpsfC | | psmUpsmU |
| 266 | 1168 | 86 | 106 | fUpsmGpsfAmGfCmGfAmGfCmAfCmGfCmCfC | 1750 | vmGpsfGpsmUfGmCfGmGfGmGfCmGfUmGfCmUfCmGfCmUfCmA |
| | | | | mCfGmCfAmCpsfC | | psmUpsmU |
| 267 | 1169 | 88 | 108 | fApsmGpsfCmGfAmGfCmAfCmGfCmCfCmCfG | 1751 | vmGpsfApsmGfGmUfGmCfGmGfGmGfCmGfUmGfCmUfCmGfCm |
| | | | | mCfAmCfCmUpsfC | | UpsmUpsmU |
| 268 | 1170 | 89 | 109 | fGpsmCpsfGmAfGmCfAmCfGmCfCmCfCmGfC | 1752 | vmGpsfGpsmAfGmGfUmGfCmGfGmGfGmCfGmUfGmCfUmCfGm |
| | | | | mAfCmCfUmCpsfC | | CpsmUpsmU |
| 269 | 1171 | 91 | 111 | fGpsmApsfGmCfAmCfGmCfCmCfCmGfCmAfC | 1753 | vmGpsfApsmGfGmAfGmGfUmGfCmGfGmGfGmCfGmUfGmCfUm |
| | | | | mCfUmCfCmUpsfC | | CpsmUpsmU |
| 270 | 1172 | 96 | 116 | fCpsmGpsfCmCfCmCfGmCfAmCfCmUfCmCfU | 1754 | vmUpsfCpsmGfCmGfGmAfGmGfAmGfGmUfGmCfGmGfGmGfCm |
| | | | | mCfCmGfCmGpsfA | | GpsmUpsmU |
| 271 | 1173 | 97 | 117 | fGpsmCpsfCmCfCmGfCmAfCmCfUmCfCmUfC | 1755 | vmGpsfUpsmCfGmCfGmGfAmGfGmAfGmGfUmGfCmGfGmGfGm |
| | | | | mCfGmCfGmApsfC | | CpsmUpsmU |
| 272 | 1174 | 98 | 118 | fCpsmCpsfCmCfGmCfAmCfCmUfCmCfUmCfC | 1756 | vmCpsfGpsmUfCmGfCmGfGmAfGmGfAmGfGmUfGmCfGmGfGm |
| | | | | mGfCmGfAmCpsfG | | GpsmUpsmU |
| 273 | 1175 | 99 | 119 | fCpsmCpsfCmGfCmAfCmCfUmCfCmUfCmCfG | 1757 | vmGpsfCpsmGfUmCfGmCfGmGfAmGfGmAfGmGfUmGfCmGfGm |
| | | | | mCfGmAfCmGpsfC | | GpsmUpsmU |
| 274 | 1176 | 100 | 120 | fCpsmCpsfGmCfAmCfCmUfCmCfUmCfCmGfC | 1758 | vmCpsfGpsmCfGmUfCmGfCmGfGmAfGmGfAmGfGmUfGmCfGmG |
| | | | | mGfAmCfGmCpsfG | | psmUpsmU |
| 275 | 1177 | 101 | 121 | fCpsmGpsfCmAfCmCfUmCfCmUfCmCfGmCfG | 1759 | vmGpsfCpsmGfCmGfUmCfGmCfGmGfAmGfGmAfGmGfUmGfCmG |
| | | | | mAfCmGfCmGpsfC | | psmUpsmU |
| 276 | 1178 | 103 | 123 | fCpsmApsfCmCfUmCfCmUfCmCfGmCfGmAfC | 1760 | vmGpsfCpsmGfCmGfCmGfUmCfGmCfGmGfAmGfGmAfGmGfUmG |
| | | | | mGfCmGfCmGpsfC | | psmUpsmU |
| 277 | 1179 | 105 | 125 | fCpsmCpsfUmCfCmUfCmCfGmCfGmAfCmGfC | 1761 | vmApsfUpsmGfCmGfCmGfCmGfUmCfGmCfGmGfAmGfGmAfGmG |
| | | | | mGfCmGfCmApsfU | | psmUpsmU |
| 278 | 1180 | 106 | 126 | fCpsmUpsfCmCfUmCfCmGfCmGfAmCfGmCfG | 1762 | vmCpsfApsmUfGmCfGmCfGmCfGmUfCmGfCmGfGmAfGmGfAmG |
| | | | | mCfGmCfAmUpsfG | | psmUpsmU |
| 279 | 1181 | 107 | 127 | fUpsmCpsfCmUfCmCfGmCfGmAfCmGfCmGfC | 1763 | vmApsfCpsmAfUmGfCmGfCmGfCmGfUmCfGmCfGmGfAmGfGmA |
| | | | | mGfCmAfUmGpsfU | | psmUpsmU |
| 280 | 1182 | 108 | 128 | fCpsmCpsfUmCfCmGfCmGfAmCfGmCfGmCfG | 1764 | vmApsfApsmCfAmUfGmCfGmCfGmCfGmUfCmGfCmGfGmAfGmG |
| | | | | mCfAmUfGmUpsfU | | psmUpsmU |
| 281 | 1183 | 109 | 129 | fCpsmUpsfCmCfGmCfGmAfCmGfCmGfCmGfC | 1765 | vmCpsfApsmAfCmAfUmGfCmGfCmGfCmGfUmCfGmCfGmGfAmG |
| | | | | mAfUmGfUmUpsfG | | psmUpsmU |
| 282 | 1184 | 110 | 130 | fUpsmCpsfCmGfCmGfAmCfGmCfGmCfGmCfA | 1766 | vmApsfCpsmAfAmCfAmUfGmCfGmCfGmCfGmUfCmGfCmGfGmA |
| | | | | mUfGmUfUmGpsfU | | psmUpsmU |
| 283 | 1185 | 111 | 131 | fCpsmCpsfGmCfGmAfCmGfCmGfCmGfCmAfU | 1767 | vmApsfApsmCfAmAfCmAfUmGfCmGfCmGfCmGfUmCfGmCfGmG |
| | | | | mGfUmUfGmUpsfU | | psmUpsmU |
| 284 | 1186 | 113 | 133 | fGpsmCpsfGmAfCmGfCmGfCmGfCmAfUmGfU | 1768 | vmCpsfGpsmAfAmCfAmAfCmAfUmGfCmGfCmGfCmGfUmCfGmC |
| | | | | mUfGmUfUmCpsfG | | psmUpsmU |
| 285 | 1187 | 114 | 134 | fCpsmGpsfAmCfGmCfGmCfGmCfAmUfGmUfU | 1769 | vmCpsfCpsmGfAmAfCmAfAmCfAmUfGmCfGmCfGmCfGmUfCmG |
| | | | | mGfUmUfCmGpsfG | | psmUpsmU |
| 286 | 1188 | 115 | 135 | fGpsmApsfCmGfCmGfCmGfCmAfUmGfUmUf | 1770 | vmGpsfCpsmCfGmAfAmCfAmAfCmAfUmGfCmGfCmGfCmGfUmC |
| | | | | GmUfUmCfGmGpsfC | | psmUpsmU |
| 287 | 1189 | 213 | 233 | fUpsmCpsfUmUfGmUfGmCfGmGfAmAfGmGf | 1771 | vmCpsfUpsmCfCmUfGmGfCmCfUmUfCmCfGmCfAmCfAmAfGmA |
| | | | | CmCfAmGfGmApsfG | | psmUpsmU |
| 288 | 1190 | 214 | 234 | fCpsmUpsfUmGfUmGfCmGfGmAfAmGfGmCf | 1772 | vmApsfCpsmUfCmCfUmGfGmCfCmUfUmCfCmGfCmAfCmAfAmG |
| | | | | CmAfGmGfAmGpsfU | | psmUpsmU |
| 289 | 1191 | 216 | 236 | fUpsmGpsfUmGfCmGfGmAfAmGfGmCfCmAf | 1773 | vmCpsfGpsmAfCmUfCmCfUmGfGmCfCmUfUmCfCmGfCmAfCmAp |
| | | | | GmGfAmGfUmCpsfG | | smUpsmU |
| 290 | 1192 | 218 | 238 | fUpsmGpsfCmGfGmAfAmGfGmCfCmAfGmGf | 1774 | vmUpsfCpsmCfGmAfCmUfCmCfUmGfGmCfCmUfUmCfCmGfCmA |
| | | | | AmGfUmCfGmGpsfA | | psmUpsmU |
| 291 | 1193 | 220 | 240 | fCpsmGpsfGmAfAmGfGmCfCmAfGmGfAmGf | 1775 | vmGpsfUpsmUfCmCfGmAfCmUfCmCfUmGfGmCfCmUfUmCfCmG |
| | | | | UmCfGmGfAmApsfC | | psmUpsmU |
| 292 | 1194 | 223 | 243 | fApsmApsfGmGfCmCfAmGfGmAfGmUfCmGf | 1776 | vmApsfApsmUfGmUfUmCfCmGfAmCfUmCfCmUfGmGfCmCfUmU |
| | | | | GmAfAmCfAmUpsfU | | psmUpsmU |
| 293 | 1195 | 224 | 244 | fApsmGpsfGmCfCmAfGmGfAmGfUmCfGmGf | 1777 | vmCpsfApsmAfUmGfUmUfCmCfGmAfCmUfCmCfUmGfGmCfCmU |
| | | | | AmAfCmAfUmUpsfG | | psmUpsmU |
| 294 | 1196 | 225 | 245 | fGpsmGpsfCmCfAmGfGmAfGmUfCmGfGmAf | 1778 | vmCpsfCpsmAfAmUfGmUfUmCfCmGfAmCfUmCfCmUfGmGfCmC |
| | | | | AmCfAmUfUmGpsfG | | psmUpsmU |
| 295 | 1197 | 229 | 249 | fApsmGpsfGmAfGmUfCmGfGmAfAmCfAmUf | 1779 | vmGpsfApsmUfGmCfCmAfAmUfGmUfUmCfCmGfAmCfUmCfCmU |
| | | | | UmGfGmCfAmUpsfC | | psmUpsmU |
| 296 | 1198 | 311 | 331 | fCpsmCpsfAmAfUmGfUmCfCmAfCmCfAmGfC | 1780 | vmApsfGpsmAfUmGfAmGfCmUfGmGfUmGfGmAfCmAfUmUfGm |
| | | | | mUfCmAfUmCpsfU | | GpsmUpsmU |
| 297 | 1199 | 312 | 332 | fCpsmApsfAmUfGmUfCmCfAmCfCmAfGmCfU | 1781 | vmGpsfApsmGfAmUfGmAfGmCfUmGfGmUfGmGfAmCfAmUfUm |
| | | | | mCfAmUfCmUpsfC | | GpsmUpsmU |
| 298 | 1200 | 314 | 334 | fApsmUpsfGmUfCmCfAmCfCmAfGmCfUmCfA | 1782 | vmCpsfGpsmGfAmGfAmUfGmAfGmCfUmGfGmUfGmGfAmCfAm |
| | | | | mUfCmUfCmCpsfG | | UpsmUpsmU |
| 299 | 1201 | 315 | 335 | fUpsmGpsfUmCfCmAfCmCfAmGfCmUfCmAfU | 1783 | vmCpsfCpsmGfGmAfGmAfUmGfAmGfCmUfGmGfUmGfGmAfCmA |
| | | | | mCfUmCfCmGpsfG | | psmUpsmU |
| 300 | 1202 | 316 | 336 | fGpsmUpsfCmCfAmCfCmAfGmCfUmCfAmUfC | 1784 | vmGpsfCpsmCfGmGfAmGfAmUfGmAfGmCfUmGfGmUfGmGfAmC |
| | | | | mUfCmCfGmGpsfC | | psmUpsmU |
| 301 | 1203 | 317 | 337 | fUpsmCpsfCmAfCmCfAmGfCmUfCmAfUmCfU | 1785 | vmUpsfGpsmCfCmGfGmAfGmAfUmGfAmGfCmUfGmGfUmGfGm |
| | | | | mCfCmGfGmCpsfA | | ApsmUpsmU |
| 302 | 1204 | 319 | 339 | fCpsmApsfCmCfAmGfCmUfCmAfUmCfUmCfC | 1786 | vmUpsfUpsmUfGmCfCmGfGmAfGmAfUmGfAmGfCmUfGmGfUm |
| | | | | mGfGmCfAmApsfA | | GpsmUpsmU |
| 303 | 1205 | 351 | 371 | fUpsmCpsfUmUfAmCfCmAfGmAfGmUfGmUf | 1787 | vmCpsfCpsmAfUmCfAmGfAmCfAmCfUmCfUmGfGmUfAmAfGmA |
| | | | | CmUfGmAfUmGpsfG | | psmUpsmU |
| 304 | 1206 | 352 | 372 | fCpsmUpsfUmAfCmCfAmGfAmGfUmGfUmCf | 1788 | vmCpsfCpsmCfAmUfCmAfGmAfCmAfCmUfCmUfGmGfUmAfAmG |
| | | | | UmGfAmUfGmGpsfG | | psmUpsmU |
| 305 | 1207 | 353 | 373 | fUpsmUpsfAmCfCmAfGmAfGmUfGmUfCmUf | 1789 | vmCpsfCpsmCfCmAfUmCfAmGfAmCfAmCfUmCfUmGfGmUfAmA |
| | | | | GmAfUmGfGmGpsfG | | psmUpsmU |
| 306 | 1208 | 354 | 374 | fUpsmApsfCmCfAmGfAmGfUmGfUmCfUmGf | 1790 | vmUpsfCpsmCfCmCfAmUfCmAfGmAfCmAfCmUfCmUfGmGfUmA |
| | | | | AmUfGmGfGmGpsfA | | psmUpsmU |
| 307 | 1209 | 357 | 377 | fCpsmApsfGmAfGmUfGmUfCmUfGmAfUmGf | 1791 | vmUpsfUpsmUfUmCfCmCfCmAfUmCfAmGfAmCfAmCfUmCfUmG |
| | | | | GmGfGmAfAmApsfA | | psmUpsmU |
| 308 | 1210 | 358 | 378 | fApsmGpsfAmGfUmGfUmCfUmGfAmUfGmGf | 1792 | vmGpsfUpsmUfUmUfCmCfCmCfAmUfCmAfGmAfCmAfCmUfCmU |
| | | | | GmGfAmAfAmApsfC | | psmUpsmU |
| 309 | 1211 | 359 | 379 | fGpsmApsfGmUfGmUfCmUfGmAfUmGfGmGf | 1793 | vmCpsfGpsmUfUmUfUmCfCmCfCmAfUmCfAmGfAmCfAmCfUmC |
| | | | | GmAfAmAfAmCpsfG | | psmUpsmU |
| 310 | 1212 | 360 | 380 | fApsmGpsfUmGfUmCfUmGfAmUfGmGfGmGf | 1794 | vmApsfCpsmGfUmUfUmUfCmCfCmCfAmUfCmAfGmAfCmAfCmU |
| | | | | AmAfAmAfCmGpsfU | | psmUpsmU |
| 311 | 1213 | 361 | 381 | fGpsmUpsfGmUfCmUfGmAfUmGfGmGfGmAf | 1795 | vmApsfApsmCfGmUfUmUfUmCfCmCfCmAfUmCfAmGfAmCfAmC |
| | | | | AmAfAmCfGmUpsfU | | psmUpsmU |
| 312 | 1214 | 362 | 382 | fUpsmGpsfUmCfUmGfAmUfGmGfGmGfAmAf | 1796 | vmGpsfApsmAfCmGfUmUfUmUfCmCfCmCfAmUfCmAfGmAfCmA |
| | | | | AmAfCmGfUmUpsfC | | psmUpsmU |
| 313 | 1215 | 363 | 383 | fGpsmUpsfCmUfGmAfUmGfGmGfGmAfAmAf | 1797 | vmApsfGpsmAfAmCfGmUfUmUfUmCfCmCfCmAfUmCfAmGfAmC |
| | | | | AmCfGmUfUmCpsfU | | psmUpsmU |
| 314 | 1216 | 364 | 384 | fUpsmCpsfUmGfAmUfGmGfGmGfAmAfAmAf | 1798 | vmCpsfApsmGfAmAfCmGfUmUfUmUfCmCfCmCfAmUfCmAfGmA |
| | | | | CmGfUmUfCmUpsfG | | psmUpsmU |
| 315 | 1217 | 365 | 385 | fCpsmUpsfGmAfUmGfGmGfGmAfAmAfAmCf | 1799 | vmCpsfCpsmAfGmAfAmCfGmUfUmUfUmCfCmCfCmAfUmCfAmG |
| | | | | GmUfUmCfUmGpsfG | | psmUpsmU |
| 316 | 1218 | 366 | 386 | fUpsmGpsfAmUfGmGfGmGfAmAfAmAfCmGf | 1800 | vmApsfCpsmCfAmGfAmAfCmGfUmUfUmUfCmCfCmCfAmUfCmA |
| | | | | UmUfCmUfGmGpsfU | | psmUpsmU |
| 317 | 1219 | 367 | 387 | fGpsmApsfUmGfGmGfGmAfAmAfAmCfGmUf | 1801 | vmCpsfApsmCfCmAfGmAfAmCfGmUfUmUfUmCfCmCfCmAfUmCp |
| | | | | UmCfUmGfGmUpsfG | | smUpsmU |
| 318 | 1220 | 369 | 389 | fUpsmGpsfGmGfGmAfAmAfAmCfGmUfUmCf | 1802 | vmGpsfApsmCfAmCfCmAfGmAfAmCfGmUfUmUfUmCfCmCfCmA |
| | | | | UmGfGmUfGmUpsfC | | psmUpsmU |
| 319 | 1221 | 371 | 391 | fGpsmGpsfGmAfAmAfAmCfGmUfUmCfUmGf | 1803 | vmCpsfApsmGfAmCfAmCfCmAfGmAfAmCfGmUfUmUfUmCfCmC |
| | | | | GmUfGmUfCmUpsfG | | psmUpsmU |
| 320 | 1222 | 372 | 392 | fGpsmGpsfAmAfAmAfCmGfUmUfCmUfGmGf | 1804 | vmUpsfCpsmAfGmAfCmAfCmCfAmGfAmAfCmGfUmUfUmUfCmC |
| | | | | UmGfUmCfUmGpsfA | | psmUpsmU |
| 321 | 1223 | 375 | 395 | fApsmApsfAmCfGmUfUmCfUmGfGmUfGmUf | 1805 | vmApsfApsmGfUmCfAmGfAmCfAmCfCmAfGmAfAmCfGmUfUmU |
| | | | | CmUfGmAfCmUpsfU | | psmUpsmU |
| 322 | 1224 | 376 | 396 | fApsmApsfCmGfUmUfCmUfGmGfUmGfUmCf | 1806 | vmApsfApsmAfGmUfCmAfGmAfCmAfCmCfAmGfAmAfCmGfUmU |
| | | | | UmGfAmCfUmUpsfU | | psmUpsmU |
| 323 | 1225 | 377 | 397 | fApsmCpsfGmUfUmCfUmGfGmUfGmUfCmUf | 1807 | vmGpsfApsmAfAmGfUmCfAmGfAmCfAmCfCmAfGmAfAmCfGmU |
| | | | | GmAfCmUfUmUpsfC | | psmUpsmU |
| 324 | 1226 | 378 | 398 | fCpsmGpsfUmUfCmUfGmGfUmGfUmCfUmGf | 1808 | vmCpsfGpsmAfAmAfGmUfCmAfGmAfCmAfCmCfAmGfAmAfCmG |
| | | | | AmCfUmUfUmCpsfG | | psmUpsmU |
| 325 | 1227 | 382 | 402 | fCpsmUpsfGmGfUmGfUmCfUmGfAmCfUmUf | 1809 | vmGpsfGpsmAfCmCfGmAfAmAfGmUfCmAfGmAfCmAfCmCfAmG |
| | | | | UmCfGmGfUmCpsfC | | psmUpsmU |
| 326 | 1228 | 383 | 403 | fUpsmGpsfGmUfGmUfCmUfGmAfCmUfUmUf | 1810 | vmUpsfGpsmGfAmCfCmGfAmAfAmGfUmCfAmGfAmCfAmCfCmA |
| | | | | CmGfGmUfCmCpsfA | | psmUpsmU |
| 327 | 1229 | 384 | 404 | fGpsmGpsfUmGfUmCfUmGfAmCfUmUfUmCf | 1811 | vmUpsfUpsmGfGmAfCmCfGmAfAmAfGmUfCmAfGmAfCmAfCmC |
| | | | | GmGfUmCfCmApsfA | | psmUpsmU |
| 328 | 1230 | 385 | 405 | fGpsmUpsfGmUfCmUfGmAfCmUfUmUfCmGf | 1812 | vmUpsfUpsmUfGmGfAmCfCmGfAmAfAmGfUmCfAmGfAmCfAmC |
| | | | | GmUfCmCfAmApsfA | | psmUpsmU |
| 329 | 1231 | 387 | 407 | fGpsmUpsfCmUfGmAfCmUfUmUfCmGfGmUf | 1813 | vmUpsfCpsmUfUmUfGmGfAmCfCmGfAmAfAmGfUmCfAmGfAmC |
| | | | | CmCfAmAfAmGpsfA | | psmUpsmU |
| 330 | 1232 | 388 | 408 | fUpsmCpsfUmGfAmCfUmUfUmCfGmGfUmCf | 1814 | vmGpsfUpsmCfUmUfUmGfGmAfCmCfGmAfAmAfGmUfCmAfGmA |
| | | | | CmAfAmAfGmApsfC | | psmUpsmU |
| 331 | 1233 | 398 | 418 | fGpsmGpsfUmCfCmAfAmAfGmAfCmGfAmAfG | 1815 | vmCpsfCpsmAfCmGfAmCfUmUfCmGfUmCfUmUfUmGfGmAfCmC |
| | | | | mUfCmGfUmGpsfG | | psmUpsmU |
| 332 | 1234 | 399 | 419 | fGpsmUpsfCmCfAmAfAmGfAmCfGmAfAmGfU | 1816 | vmUpsfCpsmCfAmCfGmAfCmUfUmCfGmUfCmUfUmUfGmGfAmC |
| | | | | mCfGmUfGmGpsfA | | psmUpsmU |
| 333 | 1235 | 400 | 420 | fUpsmCpsfCmAfAmAfGmAfCmGfAmAfGmUfC | 1817 | vmApsfUpsmCfCmAfCmGfAmCfUmUfCmGfUmCfUmUfUmGfGmA |
| | | | | mGfUmGfGmApsfU | | psmUpsmU |
| 334 | 1236 | 402 | 422 | fCpsmApsfAmAfGmAfCmGfAmAfGmUfCmGfU | 1818 | vmGpsfCpsmAfUmCfCmAfCmGfAmCfUmUfCmGfUmCfUmUfUmG |
| | | | | mGfGmAfUmGpsfC | | psmUpsmU |
| 335 | 1237 | 403 | 423 | fApsmApsfAmGfAmCfGmAfAmGfUmCfGmUf | 1819 | vmGpsfGpsmCfAmUfCmCfAmCfGmAfCmUfUmCfGmUfCmUfUmU |
| | | | | GmGfAmUfGmCpsfC | | psmUpsmU |
| 336 | 1238 | 404 | 424 | fApsmApsfGmAfCmGfAmAfGmUfCmGfUmGf | 1820 | vmApsfGpsmGfCmAfUmCfCmAfCmGfAmCfUmUfCmGfUmCfUmU |
| | | | | GmAfUmGfCmCpsfU | | psmUpsmU |
| 337 | 1239 | 405 | 425 | fApsmGpsfAmCfGmAfAmGfUmCfGmUfGmGf | 1821 | vmApsfApsmGfGmCfAmUfCmCfAmCfGmAfCmUfUmCfGmUfCmU |
| | | | | AmUfGmCfCmUpsfU | | psmUpsmU |
| 338 | 1240 | 407 | 427 | fApsmCpsfGmAfAmGfUmCfGmUfGmGfAmUf | 1822 | vmCpsfCpsmAfAmGfGmCfAmUfCmCfAmCfGmAfCmUfUmCfGmU |
| | | | | GmCfCmUfUmGpsfG | | psmUpsmU |
| 339 | 1241 | 408 | 428 | fCpsmGpsfAmAfGmUfCmGfUmGfGmAfUmGf | 1823 | vmApsfCpsmCfAmAfGmGfCmAfUmCfCmAfCmGfAmCfUmUfCmG |
| | | | | CmCfUmUfGmGpsfU | | psmUpsmU |
| 340 | 1242 | 409 | 429 | fGpsmApsfAmGfUmCfGmUfGmGfAmUfGmCf | 1824 | vmUpsfApsmCfCmAfAmGfGmCfAmUfCmCfAmCfGmAfCmUfUmC |
| | | | | CmUfUmGfGmUpsfA | | psmUpsmU |
| 341 | 1243 | 410 | 430 | fApsmApsfGmUfCmGfUmGfGmAfUmGfCmCf | 1825 | vmApsfUpsmAfCmCfAmAfGmGfCmAfUmCfCmAfCmGfAmCfUmU |
| | | | | UmUfGmGfUmApsfU | | psmUpsmU |
| 342 | 1244 | 412 | 432 | fGpsmUpsfCmGfUmGfGmAfUmGfCmCfUmUf | 1826 | vmApsfCpsmAfUmAfCmCfAmAfGmGfCmAfUmCfCmAfCmGfAmCp |
| | | | | GmGfUmAfUmGpsfU | | smUpsmU |
| 343 | 1245 | 413 | 433 | fUpsmCpsfGmUfGmGfAmUfGmCfCmUfUmGf | 1827 | vmApsfApsmCfAmUfAmCfCmAfAmGfGmCfAmUfCmCfAmCfGmAp |
| | | | | GmUfAmUfGmUpsfU | | smUpsmU |
| 344 | 1246 | 415 | 435 | fGpsmUpsfGmGfAmUfGmCfCmUfUmGfGmUf | 1828 | vmGpsfGpsmAfAmCfAmUfAmCfCmAfAmGfGmCfAmUfCmCfAmCp |
| | | | | AmUfGmUfUmCpsfC | | smUpsmU |
| 345 | 1247 | 416 | 436 | fUpsmGpsfGmAfUmGfCmCfUmUfGmGfUmAf | 1829 | vmApsfGpsmGfAmAfCmAfUmAfCmCfAmAfGmGfCmAfUmCfCmA |
| | | | | UmGfUmUfCmCpsfU | | psmUpsmU |
| 346 | 1248 | 417 | 437 | fGpsmGpsfAmUfGmCfCmUfUmGfGmUfAmUf | 1830 | vmCpsfApsmGfGmAfAmCfAmUfAmCfCmAfAmGfGmCfAmUfCmCp |
| | | | | GmUfUmCfCmUpsfG | | smUpsmU |
| 347 | 1249 | 419 | 439 | fApsmUpsfGmCfCmUfUmGfGmUfAmUfGmUf | 1831 | vmApsfGpsmCfAmGfGmAfAmCfAmUfAmCfCmAfAmGfGmCfAmU |
| | | | | UmCfCmUfGmCpsfU | | psmUpsmU |
| 348 | 1250 | 421 | 441 | fGpsmCpsfCmUfUmGfGmUfAmUfGmUfUmCf | 1832 | vmGpsfApsmAfGmCfAmGfGmAfAmCfAmUfAmCfCmAfAmGfGmC |
| | | | | CmUfGmCfUmUpsfC | | psmUpsmU |
| 349 | 1251 | 430 | 450 | fUpsmGpsfUmUfCmCfUmGfCmUfUmCfAmUf | 1833 | vmGpsfApsmAfGmGfGmCfAmUfGmAfAmGfCmAfGmGfAmAfCmA |
| | | | | GmCfCmCfUmUpsfC | | psmUpsmU |
| 350 | 1252 | 433 | 453 | fUpsmCpsfCmUfGmCfUmUfCmAfUmGfCmCfC | 1834 | vmGpsfUpsmAfGmAfAmGfGmGfCmAfUmGfAmAfGmCfAmGfGmA |
| | | | | mUfUmCfUmApsfC | | psmUpsmU |
| 351 | 1253 | 454 | 474 | fApsmGpsfUmGfGmCfCmUfUmAfUmCfCmCfU | 1835 | vmGpsfGpsmAfAmGfGmAfGmGfGmAfUmAfAmGfGmCfCmAfCmU |
| | | | | mCfCmUfUmCpsfC | | psmUpsmU |
| 352 | 1254 | 457 | 477 | fGpsmGpsfCmCfUmUfAmUfCmCfCmUfCmCfU | 1836 | vmGpsfApsmAfGmGfAmAfGmGfAmGfGmGfAmUfAmAfGmGfCmC |
| | | | | mUfCmCfUmUpsfC | | psmUpsmU |
| 353 | 1255 | 459 | 479 | fCpsmCpsfUmUfAmUfCmCfCmUfCmCfUmUfC | 1837 | vmCpsfUpsmGfAmAfGmGfAmAfGmGfAmGfGmGfAmUfAmAfGm |
| | | | | mCfUmUfCmApsfG | | GpsmUpsmU |
| 354 | 1256 | 460 | 480 | fCpsmUpsfUmAfUmCfCmCfUmCfCmUfUmCfC | 1838 | vmUpsfCpsmUfGmAfAmGfGmAfAmGfGmAfGmGfGmAfUmAfAm |
| | | | | mUfUmCfAmGpsfA | | GpsmUpsmU |
| 355 | 1257 | 461 | 481 | fUpsmUpsfAmUfCmCfCmUfCmCfUmUfCmCfU | 1839 | vmCpsfUpsmCfUmGfAmAfGmGfAmAfGmGfAmGfGmGfAmUfAmA |
| | | | | mUfCmAfGmApsfG | | psmUpsmU |
| 356 | 1258 | 465 | 485 | fCpsmCpsfCmUfCmCfUmUfCmCfUmUfCmAfG | 1840 | vmApsfCpsmGfCmCfUmCfUmGfAmAfGmGfAmAfGmGfAmGfGmG |
| | | | | mAfGmGfCmGpsfU | | psmUpsmU |
| 357 | 1259 | 466 | 486 | fCpsmCpsfUmCfCmUfUmCfCmUfUmCfAmGfA | 1841 | vmCpsfApsmCfGmCfCmUfCmUfGmAfAmGfGmAfAmGfGmAfGmG |
| | | | | mGfGmCfGmUpsfG | | psmUpsmU |
| 358 | 1260 | 467 | 487 | fCpsmUpsfCmCfUmUfCmCfUmUfCmAfGmAfG | 1842 | vmGpsfCpsmAfCmGfCmCfUmCfUmGfAmAfGmGfAmAfGmGfAmG |
| | | | | mGfCmGfUmGpsfC | | psmUpsmU |
| 359 | 1261 | 469 | 489 | fCpsmCpsfUmUfCmCfUmUfCmAfGmAfGmGfC | 1843 | vmUpsfCpsmGfCmAfCmGfCmCfUmCfUmGfAmAfGmGfAmAfGmG |
| | | | | mGfUmGfCmGpsfA | | psmUpsmU |
| 360 | 1262 | 470 | 490 | fCpsmUpsfUmCfCmUfUmCfAmGfAmGfGmCfG | 1844 | vmApsfUpsmCfGmCfAmCfGmCfCmUfCmUfGmAfAmGfGmAfAmG |
| | | | | mUfGmCfGmApsfU | | psmUpsmU |
| 361 | 1263 | 471 | 491 | fUpsmUpsfCmCfUmUfCmAfGmAfGmGfCmGf | 1845 | vmUpsfApsmUfCmGfCmAfCmGfCmCfUmCfUmGfAmAfGmGfAmA |
| | | | | UmGfCmGfAmUpsfA | | psmUpsmU |
| 362 | 1264 | 489 | 509 | fApsmUpsfAmUfGmUfGmGfAmUfGmGfAmGf | 1846 | vmCpsfUpsmCfAmCfUmCfCmUfCmCfAmUfCmCfAmCfAmUfAmUp |
| | | | | GmAfGmUfGmApsfG | | smUpsmU |
| 363 | 1265 | 499 | 519 | fGpsmGpsfAmGfGmAfGmUfGmAfGmUfGmAf | 1847 | vmUpsfApsmCfGmUfUmGfUmCfAmCfUmCfAmCfUmCfCmUfCmC |
| | | | | CmAfAmCfGmUpsfA | | psmUpsmU |
| 364 | 1266 | 501 | 521 | fApsmGpsfGmAfGmUfGmAfGmUfGmAfCmAf | 1848 | vmGpsfGpsmUfAmCfGmUfUmGfUmCfAmCfUmCfAmCfUmCfCmU |
| | | | | AmCfGmUfAmCpsfC | | psmUpsmU |
| 365 | 1267 | 502 | 522 | fGpsmGpsfAmGfUmGfAmGfUmGfAmCfAmAf | 1849 | vmGpsfGpsmGfUmAfCmGfUmUfGmUfCmAfCmUfCmAfCmUfCmC |
| | | | | CmGfUmAfCmCpsfC | | psmUpsmU |
| 366 | 1268 | 505 | 525 | fGpsmUpsfGmAfGmUfGmAfCmAfAmCfGmUf | 1850 | vmGpsfApsmAfGmGfGmUfAmCfGmUfUmGfUmCfAmCfUmCfAmC |
| | | | | AmCfCmCfUmUpsfC | | psmUpsmU |
| 367 | 1269 | 528 | 548 | fUpsmGpsfAmUfGmCfCmAfAmAfAmCfAmAfC | 1851 | vmGpsfUpsmGfAmUfGmGfUmUfGmUfUmUfUmGfGmCfAmUfCm |
| | | | | mCfAmUfCmApsfC | | ApsmUpsmU |
| 368 | 1270 | 529 | 549 | fGpsmApsfUmGfCmCfAmAfAmAfCmAfAmCfC | 1852 | vmGpsfGpsmUfGmAfUmGfGmUfUmGfUmUfUmUfGmGfCmAfUm |
| | | | | mAfUmCfAmCpsfC | | CpsmUpsmU |
| 369 | 1271 | 530 | 550 | fApsmUpsfGmCfCmAfAmAfAmCfAmAfCmCfA | 1853 | vmCpsfGpsmGfUmGfAmUfGmGfUmUfGmUfUmUfUmGfGmCfAm |
| | | | | mUfCmAfCmCpsfG | | UpsmUpsmU |
| 370 | 1272 | 533 | 553 | fCpsmCpsfAmAfAmAfCmAfAmCfCmAfUmCfA | 1854 | vmApsfCpsmAfCmGfGmUfGmAfUmGfGmUfUmGfUmUfUmUfGm |
| | | | | mCfCmGfUmGpsfU | | GpsmUpsmU |
| 371 | 1273 | 534 | 554 | fCpsmApsfAmAfAmCfAmAfCmCfAmUfCmAfC | 1855 | vmGpsfApsmCfAmCfGmGfUmGfAmUfGmGfUmUfGmUfUmUfUm |
| | | | | mCfGmUfGmUpsfC | | GpsmUpsmU |
| 372 | 1274 | 536 | 556 | fApsmApsfAmCfAmAfCmCfAmUfCmAfCmCfG | 1856 | vmGpsfGpsmGfAmCfAmCfGmGfUmGfAmUfGmGfUmUfGmUfUm |
| | | | | mUfGmUfCmCpsfC | | UpsmUpsmU |
| 373 | 1275 | 537 | 557 | fApsmApsfCmAfAmCfCmAfUmCfAmCfCmGfU | 1857 | vmGpsfGpsmGfGmAfCmAfCmGfGmUfGmAfUmGfGmUfUmGfUm |
| | | | | mGfUmCfCmCpsfC | | UpsmUpsmU |
| 374 | 1276 | 538 | 558 | fApsmCpsfAmAfCmCfAmUfCmAfCmCfGmUfG | 1858 | vmGpsfGpsmGfGmGfAmCfAmCfGmGfUmGfAmUfGmGfUmUfGm |
| | | | | mUfCmCfCmCpsfC | | UpsmUpsmU |
| 375 | 1277 | 539 | 559 | fCpsmApsfAmCfCmAfUmCfAmCfCmGfUmGfU | 1859 | vmApsfGpsmGfGmGfGmAfCmAfCmGfGmUfGmAfUmGfGmUfUm |
| | | | | mCfCmCfCmCpsfU | | GpsmUpsmU |
| 376 | 1278 | 544 | 564 | fApsmUpsfCmAfCmCfGmUfGmUfCmCfCmCfC | 1860 | vmApsfUpsmAfGmAfAmGfGmGfGmGfAmCfAmCfGmGfUmGfAm |
| | | | | mUfUmCfUmApsfU | | UpsmUpsmU |
| 377 | 1279 | 545 | 565 | fUpsmCpsfAmCfCmGfUmGfUmCfCmCfCmCfU | 1861 | vmCpsfApsmUfAmGfAmAfGmGfGmGfGmAfCmAfCmGfGmUfGmA |
| | | | | mUfCmUfAmUpsfG | | psmUpsmU |
| 378 | 1280 | 546 | 566 | fCpsmApsfCmCfGmUfGmUfCmCfCmCfCmUfU | 1862 | vmCpsfCpsmAfUmAfGmAfAmGfGmGfGmGfAmCfAmCfGmGfUmG |
| | | | | mCfUmAfUmGpsfG | | psmUpsmU |
| 379 | 1281 | 547 | 567 | fApsmCpsfCmGfUmGfUmCfCmCfCmCfUmUfC | 1863 | vmCpsfCpsmCfAmUfAmGfAmAfGmGfGmGfGmAfCmAfCmGfGmU |
| | | | | mUfAmUfGmGpsfG | | psmUpsmU |
| 380 | 1282 | 550 | 570 | fGpsmUpsfGmUfCmCfCmCfCmUfUmCfUmAfU | 1864 | vmCpsfUpsmCfCmCfCmAfUmAfGmAfAmGfGmGfGmGfAmCfAmC |
| | | | | mGfGmGfGmApsfG | | psmUpsmU |
| 381 | 1283 | 551 | 571 | fUpsmGpsfUmCfCmCfCmCfUmUfCmUfAmUfG | 1865 | vmApsfCpsmUfCmCfCmCfAmUfAmGfAmAfGmGfGmGfGmAfCmA |
| | | | | mGfGmGfAmGpsfU | | psmUpsmU |
| 382 | 1284 | 552 | 572 | fGpsmUpsfCmCfCmCfCmUfUmCfUmAfUmGfG | 1866 | vmUpsfApsmCfUmCfCmCfCmAfUmAfGmAfAmGfGmGfGmGfAmC |
| | | | | mGfGmAfGmUpsfA | | psmUpsmU |
| 383 | 1285 | 553 | 573 | fUpsmCpsfCmCfCmCfUmUfCmUfAmUfGmGfG | 1867 | vmGpsfUpsmAfCmUfCmCfCmCfAmUfAmGfAmAfGmGfGmGfGmA |
| | | | | mGfAmGfUmApsfC | | psmUpsmU |
| 384 | 1286 | 556 | 576 | fCpsmCpsfCmUfUmCfUmAfUmGfGmGfGmAf | 1868 | vmGpsfUpsmCfGmUfAmCfUmCfCmCfCmAfUmAfGmAfAmGfGmG |
| | | | | GmUfAmCfGmApsfC | | psmUpsmU |
| 385 | 1287 | 557 | 577 | fCpsmCpsfUmUfCmUfAmUfGmGfGmGfAmGf | 1869 | vmUpsfGpsmUfCmGfUmAfCmUfCmCfCmCfAmUfAmGfAmAfGmG |
| | | | | UmAfCmGfAmCpsfA | | psmUpsmU |
| 386 | 1288 | 559 | 579 | fUpsmUpsfCmUfAmUfGmGfGmGfAmGfUmAf | 1870 | vmGpsfApsmUfGmUfCmGfUmAfCmUfCmCfCmCfAmUfAmGfAmA |
| | | | | CmGfAmCfAmUpsfC | | psmUpsmU |
| 387 | 1289 | 560 | 580 | fUpsmCpsfUmAfUmGfGmGfGmAfGmUfAmCf | 1871 | vmApsfGpsmAfUmGfUmCfGmUfAmCfUmCfCmCfCmAfUmAfGmA |
| | | | | GmAfCmAfUmCpsfU | | psmUpsmU |
| 388 | 1290 | 561 | 581 | fCpsmUpsfAmUfGmGfGmGfAmGfUmAfCmGf | 1872 | vmCpsfApsmGfAmUfGmUfCmGfUmAfCmUfCmCfCmCfAmUfAmG |
| | | | | AmCfAmUfCmUpsfG | | psmUpsmU |
| 389 | 1291 | 562 | 582 | fUpsmApsfUmGfGmGfGmAfGmUfAmCfGmAf | 1873 | vmGpsfCpsmAfGmAfUmGfUmCfGmUfAmCfUmCfCmCfCmAfUmA |
| | | | | CmAfUmCfUmGpsfC | | psmUpsmU |
| 390 | 1292 | 572 | 592 | fApsmCpsfGmAfCmAfUmCfUmGfCmCfCmUfA | 1874 | vmUpsfGpsmAfCmUfUmUfAmGfGmGfCmAfGmAfUmGfUmCfGm |
| | | | | mAfAmGfUmCpsfA | | UpsmUpsmU |
| 391 | 1293 | 573 | 593 | fCpsmGpsfAmCfAmUfCmUfGmCfCmCfUmAfA | 1875 | vmUpsfUpsmGfAmCfUmUfUmAfGmGfGmCfAmGfAmUfGmUfCm |
| | | | | mAfGmUfCmApsfA | | GpsmUpsmU |
| 392 | 1294 | 574 | 594 | fGpsmApsfCmAfUmCfUmGfCmCfCmUfAmAfA | 1876 | vmCpsfUpsmUfGmAfCmUfUmUfAmGfGmGfCmAfGmAfUmGfUm |
| | | | | mGfUmCfAmApsfG | | CpsmUpsmU |
| 393 | 1295 | 575 | 595 | fApsmCpsfAmUfCmUfGmCfCmCfUmAfAmAfG | 1877 | vmApsfCpsmUfUmGfAmCfUmUfUmAfGmGfGmCfAmGfAmUfGm |
| | | | | mUfCmAfAmGpsfU | | UpsmUpsmU |
| 394 | 1296 | 576 | 596 | fCpsmApsfUmCfUmGfCmCfCmUfAmAfAmGfU | 1878 | vmGpsfApsmCfUmUfGmAfCmUfUmUfAmGfGmGfCmAfGmAfUm |
| | | | | mCfAmAfGmUpsfC | | GpsmUpsmU |
| 395 | 1297 | 577 | 597 | fApsmUpsfCmUfGmCfCmCfUmAfAmAfGmUfC | 1879 | vmGpsfGpsmAfCmUfUmGfAmCfUmUfUmAfGmGfGmCfAmGfAm |
| | | | | mAfAmGfUmCpsfC | | UpsmUpsmU |
| 396 | 1298 | 582 | 602 | fCpsmCpsfCmUfAmAfAmGfUmCfAmAfGmUfC | 1880 | vmUpsfUpsmCfGmUfGmGfAmCfUmUfGmAfCmUfUmUfAmGfGm |
| | | | | mCfAmCfGmApsfA | | GpsmUpsmU |
| 397 | 1299 | 583 | 603 | fCpsmCpsfUmAfAmAfGmUfCmAfAmGfUmCfC | 1881 | vmGpsfUpsmUfCmGfUmGfGmAfCmUfUmGfAmCfUmUfUmAfGm |
| | | | | mAfCmGfAmApsfC | | GpsmUpsmU |
| 398 | 1300 | 585 | 605 | fUpsmApsfAmAfGmUfCmAfAmGfUmCfCmAfC | 1882 | vmApsfApsmGfUmUfCmGfUmGfGmAfCmUfUmGfAmCfUmUfUm |
| | | | | mGfAmAfCmUpsfU | | ApsmUpsmU |
| 399 | 1301 | 586 | 606 | fApsmApsfAmGfUmCfAmAfGmUfCmCfAmCfG | 1883 | vmApsfApsmAfGmUfUmCfGmUfGmGfAmCfUmUfGmAfCmUfUm |
| | | | | mAfAmCfUmUpsfU | | UpsmUpsmU |
| 400 | 1302 | 587 | 607 | fApsmApsfGmUfCmAfAmGfUmCfCmAfCmGfA | 1884 | vmGpsfApsmAfAmGfUmUfCmGfUmGfGmAfCmUfUmGfAmCfUm |
| | | | | mAfCmUfUmUpsfC | | UpsmUpsmU |
| 401 | 1303 | 588 | 608 | fApsmGpsfUmCfAmAfGmUfCmCfAmCfGmAfA | 1885 | vmApsfGpsmAfAmAfGmUfUmCfGmUfGmGfAmCfUmUfGmAfCm |
| | | | | mCfUmUfUmCpsfU | | UpsmUpsmU |
| 402 | 1304 | 589 | 609 | fGpsmUpsfCmAfAmGfUmCfCmAfCmGfAmAfC | 1886 | vmApsfApsmGfAmAfAmGfUmUfCmGfUmGfGmAfCmUfUmGfAmC |
| | | | | mUfUmUfCmUpsfU | | psmUpsmU |
| 403 | 1305 | 590 | 610 | fUpsmCpsfAmAfGmUfCmCfAmCfGmAfAmCfU | 1887 | vmGpsfApsmAfGmAfAmAfGmUfUmCfGmUfGmGfAmCfUmUfGm |
| | | | | mUfUmCfUmUpsfC | | ApsmUpsmU |
| 404 | 1306 | 591 | 611 | fCpsmApsfAmGfUmCfCmAfCmGfAmAfCmUfU | 1888 | vmUpsfGpsmAfAmGfAmAfAmGfUmUfCmGfUmGfGmAfCmUfUm |
| | | | | mUfCmUfUmCpsfA | | GpsmUpsmU |
| 405 | 1307 | 592 | 612 | fApsmApsfGmUfCmCfAmCfGmAfAmCfUmUfU | 1889 | vmApsfUpsmGfAmAfGmAfAmAfGmUfUmCfGmUfGmGfAmCfUm |
| | | | | mCfUmUfCmApsfU | | UpsmUpsmU |
| 406 | 1308 | 593 | 613 | fApsmGpsfUmCfCmAfCmGfAmAfCmUfUmUfC | 1890 | vmCpsfApsmUfGmAfAmGfAmAfAmGfUmUfCmGfUmGfGmAfCmU |
| | | | | mUfUmCfAmUpsfG | | psmUpsmU |
| 407 | 1309 | 595 | 615 | fUpsmCpsfCmAfCmGfAmAfCmUfUmUfCmUfU | 1891 | vmCpsfApsmCfAmUfGmAfAmGfAmAfAmGfUmUfCmGfUmGfGmA |
| | | | | mCfAmUfGmUpsfG | | psmUpsmU |
| 408 | 1310 | 618 | 638 | fCpsmApsfUmCfAmCfCmAfAmGfCmUfCmAfG | 1892 | vmCpsfGpsmUfAmGfAmCfUmGfAmGfCmUfUmGfGmUfGmAfUm |
| | | | | mUfCmUfAmCpsfG | | GpsmUpsmU |
| 409 | 1311 | 619 | 639 | fApsmUpsfCmAfCmCfAmAfGmCfUmCfAmGfU | 1893 | vmGpsfCpsmGfUmAfGmAfCmUfGmAfGmCfUmUfGmGfUmGfAm |
| | | | | mCfUmAfCmGpsfC | | UpsmUpsmU |
| 410 | 1312 | 620 | 640 | fUpsmCpsfAmCfCmAfAmGfCmUfCmAfGmUfC | 1894 | vmGpsfGpsmCfGmUfAmGfAmCfUmGfAmGfCmUfUmGfGmUfGm |
| | | | | mUfAmCfGmCpsfC | | ApsmUpsmU |
| 411 | 1313 | 621 | 641 | fCpsmApsfCmCfAmAfGmCfUmCfAmGfUmCfU | 1895 | vmApsfGpsmGfCmGfUmAfGmAfCmUfGmAfGmCfUmUfGmGfUm |
| | | | | mAfCmGfCmCpsfU | | GpsmUpsmU |
| 412 | 1314 | 622 | 642 | fApsmCpsfCmAfAmGfCmUfCmAfGmUfCmUfA | 1896 | vmGpsfApsmGfGmCfGmUfAmGfAmCfUmGfAmGfCmUfUmGfGm |
| | | | | mCfGmCfCmUpsfC | | UpsmUpsmU |
| 413 | 1315 | 623 | 643 | fCpsmCpsfAmAfGmCfUmCfAmGfUmCfUmAfC | 1897 | vmApsfGpsmAfGmGfCmGfUmAfGmAfCmUfGmAfGmCfUmUfGm |
| | | | | mGfCmCfUmCpsfU | | GpsmUpsmU |
| 414 | 1316 | 624 | 644 | fCpsmApsfAmGfCmUfCmAfGmUfCmUfAmCfG | 1898 | vmCpsfApsmGfAmGfGmCfGmUfAmGfAmCfUmGfAmGfCmUfUmG |
| | | | | mCfCmUfCmUpsfG | | psmUpsmU |
| 415 | 1317 | 628 | 648 | fCpsmUpsfCmAfGmUfCmUfAmCfGmCfCmUfC | 1899 | vmUpsfGpsmUfGmCfAmGfAmGfGmCfGmUfAmGfAmCfUmGfAm |
| | | | | mUfGmCfAmCpsfA | | GpsmUpsmU |
| 416 | 1318 | 629 | 649 | fUpsmCpsfAmGfUmCfUmAfCmGfCmCfUmCfU | 1900 | vmCpsfUpsmGfUmGfCmAfGmAfGmGfCmGfUmAfGmAfCmUfGmA |
| | | | | mGfCmAfCmApsfG | | psmUpsmU |
| 417 | 1319 | 630 | 650 | fCpsmApsfGmUfCmUfAmCfGmCfCmUfCmUfG | 1901 | vmCpsfCpsmUfGmUfGmCfAmGfAmGfGmCfGmUfAmGfAmCfUmG |
| | | | | mCfAmCfAmGpsfG | | psmUpsmU |
| 418 | 1320 | 636 | 656 | fApsmCpsfGmCfCmUfCmUfGmCfAmCfAmGfG | 1902 | vmApsfGpsmGfUmUfCmCfCmUfGmUfGmCfAmGfAmGfGmCfGmU |
| | | | | mGfAmAfCmCpsfU | | psmUpsmU |
| 419 | 1321 | 637 | 657 | fCpsmGpsfCmCfUmCfUmGfCmAfCmAfGmGfG | 1903 | vmGpsfApsmGfGmUfUmCfCmCfUmGfUmGfCmAfGmAfGmGfCmG |
| | | | | mAfAmCfCmUpsfC | | psmUpsmU |
| 420 | 1322 | 638 | 658 | fGpsmCpsfCmUfCmUfGmCfAmCfAmGfGmGfA | 1904 | vmApsfGpsmAfGmGfUmUfCmCfCmUfGmUfGmCfAmGfAmGfGmC |
| | | | | mAfCmCfUmCpsfU | | psmUpsmU |
| 421 | 1323 | 640 | 660 | fCpsmUpsfCmUfGmCfAmCfAmGfGmGfAmAfC | 1905 | vmGpsfUpsmAfGmAfGmGfUmUfCmCfCmUfGmUfGmCfAmGfAm |
| | | | | mCfUmCfUmApsfC | | GpsmUpsmU |
| 422 | 1324 | 641 | 661 | fUpsmCpsfUmGfCmAfCmAfGmGfGmAfAmCfC | 1906 | vmGpsfGpsmUfAmGfAmGfGmUfUmCfCmCfUmGfUmGfCmAfGm |
| | | | | mUfCmUfAmCpsfC | | ApsmUpsmU |
| 423 | 1325 | 644 | 664 | fGpsmCpsfAmCfAmGfGmGfAmAfCmCfUmCfU | 1907 | vmGpsfApsmAfGmGfUmAfGmAfGmGfUmUfCmCfCmUfGmUfGm |
| | | | | mAfCmCfUmUpsfC | | CpsmUpsmU |
| 424 | 1326 | 645 | 665 | fCpsmApsfCmAfGmGfGmAfAmCfCmUfCmUfA | 1908 | vmApsfGpsmAfAmGfGmUfAmGfAmGfGmUfUmCfCmCfUmGfUm |
| | | | | mCfCmUfUmCpsfU | | GpsmUpsmU |
| 425 | 1327 | 694 | 714 | fApsmApsfGmGfUmGfCmUfGmGfGmAfGmAf | 1909 | vmGpsfCpsmAfUmAfUmCfUmCfUmCfCmCfAmGfCmAfCmCfUmU |
| | | | | GmAfUmAfUmGpsfC | | psmUpsmU |
| 426 | 1328 | 695 | 715 | fApsmGpsfGmUfGmCfUmGfGmGfAmGfAmGf | 1910 | vmGpsfGpsmCfAmUfAmUfCmUfCmUfCmCfCmAfGmCfAmCfCmU |
| | | | | AmUfAmUfGmCpsfC | | psmUpsmU |
| 427 | 1329 | 696 | 716 | fGpsmGpsfUmGfCmUfGmGfGmAfGmAfGmAf | 1911 | vmApsfGpsmGfCmAfUmAfUmCfUmCfUmCfCmCfAmGfCmAfCmCp |
| | | | | UmAfUmGfCmCpsfU | | smUpsmU |
| 428 | 1330 | 697 | 717 | fGpsmUpsfGmCfUmGfGmGfAmGfAmGfAmUf | 1912 | vmApsfApsmGfGmCfAmUfAmUfCmUfCmUfCmCfCmAfGmCfAmC |
| | | | | AmUfGmCfCmUpsfU | | psmUpsmU |
| 429 | 1331 | 735 | 755 | fApsmUpsfUmCfAmGfGmUfUmCfUmUfGmGf | 1913 | vmUpsfUpsmCfUmCfUmUfCmCfAmAfGmAfAmCfCmUfGmAfAmU |
| | | | | AmAfGmAfGmApsfA | | psmUpsmU |
| 430 | 1332 | 741 | 761 | fGpsmUpsfUmCfUmUfGmGfAmAfGmAfGmAf | 1914 | vmApsfUpsmGfCmCfCmUfUmCfUmCfUmUfCmCfAmAfGmAfAmC |
| | | | | AmGfGmGfCmApsfU | | psmUpsmU |
| 431 | 1333 | 742 | 762 | fUpsmUpsfCmUfUmGfGmAfAmGfAmGfAmAf | 1915 | vmGpsfApsmUfGmCfCmCfUmUfCmUfCmUfUmCfCmAfAmGfAmA |
| | | | | GmGfGmCfAmUpsfC | | psmUpsmU |
| 432 | 1334 | 743 | 763 | fUpsmCpsfUmUfGmGfAmAfGmAfGmAfAmGf | 1916 | vmApsfGpsmAfUmGfCmCfCmUfUmCfUmCfUmUfCmCfAmAfGmA |
| | | | | GmGfCmAfUmCpsfU | | psmUpsmU |
| 433 | 1335 | 744 | 764 | fCpsmUpsfUmGfGmAfAmGfAmGfAmAfGmGf | 1917 | vmCpsfApsmGfAmUfGmCfCmCfUmUfCmUfCmUfUmCfCmAfAmG |
| | | | | GmCfAmUfCmUpsfG | | psmUpsmU |
| 434 | 1336 | 745 | 765 | fUpsmUpsfGmGfAmAfGmAfGmAfAmGfGmGf | 1918 | vmGpsfCpsmAfGmAfUmGfCmCfCmUfUmCfUmCfUmUfCmCfAmA |
| | | | | CmAfUmCfUmGpsfC | | psmUpsmU |
| 435 | 1337 | 747 | 767 | fGpsmGpsfAmAfGmAfGmAfAmGfGmGfCmAf | 1919 | vmUpsfUpsmGfCmAfGmAfUmGfCmCfCmUfUmCfUmCfUmUfCmC |
| | | | | UmCfUmGfCmApsfA | | psmUpsmU |
| 436 | 1338 | 748 | 768 | fGpsmApsfAmGfAmGfAmAfGmGfGmCfAmUf | 1920 | vmGpsfUpsmUfGmCfAmGfAmUfGmCfCmCfUmUfCmUfCmUfUmC |
| | | | | CmUfGmCfAmApsfC | | psmUpsmU |
| 437 | 1339 | 749 | 769 | fApsmApsfGmAfGmAfAmGfGmGfCmAfUmCf | 1921 | vmUpsfGpsmUfUmGfCmAfGmAfUmGfCmCfCmUfUmCfUmCfUmU |
| | | | | UmGfCmAfAmCpsfA | | psmUpsmU |
| 438 | 1340 | 752 | 772 | fApsmGpsfAmAfGmGfGmCfAmUfCmUfGmCfA | 1922 | vmGpsfCpsmCfUmGfUmUfGmCfAmGfAmUfGmCfCmCfUmUfCmU |
| | | | | mAfCmAfGmGpsfC | | psmUpsmU |
| 439 | 1341 | 753 | 773 | fGpsmApsfAmGfGmGfCmAfUmCfUmGfCmAfA | 1923 | vmGpsfGpsmCfCmUfGmUfUmGfCmAfGmAfUmGfCmCfCmUfUmC |
| | | | | mCfAmGfGmCpsfC | | psmUpsmU |
| 440 | 1342 | 758 | 778 | fGpsmCpsfAmUfCmUfGmCfAmAfCmAfGmGfC | 1924 | vmGpsfCpsmUfGmGfGmGfCmCfUmGfUmUfGmCfAmGfAmUfGm |
| | | | | mCfCmCfAmGpsfC | | CpsmUpsmU |
| 441 | 1343 | 1026 | 1046 | fUpsmUpsfGmCfAmAfCmUfUmGfCmUfAmCfC | 1925 | vmCpsfUpsmAfAmUfGmGfGmUfAmGfCmAfAmGfUmUfGmCfAmA |
| | | | | mCfAmUfUmApsfG | | psmUpsmU |
| 442 | 1344 | 1039 | 1059 | fCpsmCpsfCmAfUmUfAmGfGmAfUmAfAmUfG | 1926 | vmApsfUpsmAfAmGfAmCfAmUfUmAfUmCfCmUfAmAfUmGfGmG |
| | | | | mUfCmUfUmApsfU | | psmUpsmU |
| 443 | 1345 | 1062 | 1082 | fApsmApsfUmGfCmUfGmCfCmCfUmGfUmAfC | 1927 | vmGpsfGpsmCfAmGfGmGfUmAfCmAfGmGfGmCfAmGfCmAfUmU |
| | | | | mCfCmUfGmCpsfC | | psmUpsmU |
| 444 | 1346 | 1067 | 1087 | fUpsmGpsfCmCfCmUfGmUfAmCfCmCfUmGfC | 1928 | vmCpsfCpsmAfCmAfGmGfCmAfGmGfGmUfAmCfAmGfGmGfCmA |
| | | | | mCfUmGfUmGpsfG | | psmUpsmU |
| 445 | 1347 | 1068 | 1088 | fGpsmCpsfCmCfUmGfUmAfCmCfCmUfGmCfC | 1929 | vmUpsfCpsmCfAmCfAmGfGmCfAmGfGmGfUmAfCmAfGmGfGmC |
| | | | | mUfGmUfGmGpsfA | | psmUpsmU |
| 446 | 1348 | 1077 | 1097 | fCpsmCpsfUmGfCmCfUmGfUmGfGmAfAmUfC | 1930 | vmApsfUpsmGfGmCfAmGfAmUfUmCfCmAfCmAfGmGfCmAfGmG |
| | | | | mUfGmCfCmApsfU | | psmUpsmU |
| 447 | 1349 | 1080 | 1100 | fGpsmCpsfCmUfGmUfGmGfAmAfUmCfUmGf | 1931 | vmGpsfCpsmAfAmUfGmGfCmAfGmAfUmUfCmCfAmCfAmGfGmC |
| | | | | CmCfAmUfUmGpsfC | | psmUpsmU |
| 448 | 1350 | 1159 | 1179 | fCpsmApsfGmUfGmGfGmUfGmAfCmCfUmCfA | 1932 | vmCpsfApsmCfCmUfGmUfGmAfGmGfUmCfAmCfCmCfAmCfUmG |
| | | | | mCfAmGfGmUpsfG | | psmUpsmU |
| 449 | 1351 | 1195 | 1215 | fApsmUpsfGmUfGmUfCmUfGmCfUmCfCmCfC | 1933 | vmGpsfGpsmAfGmGfCmGfGmGfGmAfGmCfAmGfAmCfAmCfAmU |
| | | | | mGfCmCfUmCpsfC | | psmUpsmU |
| 450 | 1352 | 1196 | 1216 | fUpsmGpsfUmGfUmCfUmGfCmUfCmCfCmCfG | 1934 | vmUpsfGpsmGfAmGfGmCfGmGfGmGfAmGfCmAfGmAfCmAfCmA |
| | | | | mCfCmUfCmCpsfA | | psmUpsmU |
| 451 | 1353 | 497 | 515 | mApsmUpsmGmGfAmGfGfAfGmUmGmAmG | 1935 | mUpsfUpsmGmUmCfAmCmUmCmAmCmUmCfCmUfCmCmAmUp |
| | | | | mUmGmAmCmAmApsmUpsmU | | smUpsmU |
| 452 | 1354 | 419 | 437 | mApsmUpsmGmCfCmUfUfGfGmUmAmUmG | 1936 | mCpsfApsmGmGmAfAmCmAmUmAmCmCmAfAmGfGmCmAmUp |
| | | | | mUmUmCmCmUmGpsmUpsmU | | smUpsmU |
| 453 | 1355 | 377 | 395 | mApsmCpsmGmUfUmCfUfGfGmUmGmUmC | 1937 | mApsfApsmGmUmCfAmGmAmCmAmCmCmAfGmAfAmCmGmUp |
| | | | | mUmGmAmCmUmUpsmUpsmU | | smUpsmU |
| 454 | 1356 | 386 | 404 | mUpsmGpsmUmCfUmGfAfCfUmUmUmCmG | 1938 | mUpsfUpsmGmGmAfCmCmGmAmAmAmGmUfCmAfGmAmCmA |
| | | | | mGmUmCmCmAmApsmUpsmU | | psmUpsmU |
| 455 | 1357 | 398 | 416 | mGpsmGpsmUmCfCmAfAfAfGmAmCmGmAm | 1939 | mApsfCpsmGmAmCfUmUmCmGmUmCmUmUfUmGfGmAmCmC |
| | | | | AmGmUmCmGmUpsmUpsmU | | psmUpsmU |
| 456 | 1358 | 502 | 520 | mGpsmGpsmAmGfUmGfAfGfUmGmAmCmA | 1940 | mGpsfUpsmAmCmGfUmUmGmUmCmAmCmUfCmAfCmUmCmCp |
| | | | | mAmCmGmUmAmCpsmUpsmU | | smUpsmU |
| 457 | 1359 | 532 | 550 | mGpsmCpsmCmAfAmAfAfCfAmAmCmCmAm | 1941 | mCpsfGpsmGmUmGfAmUmGmGmUmUmGmUfUmUfUmGmGm |
| | | | | UmCmAmCmCmGpsmUpsmU | | CpsmUpsmU |
| 458 | 1360 | 382 | 400 | mCpsmUpsmGmGfUmGfUfCfUmGmAmCmU | 1942 | mApsfCpsmCmGmAfAmAmGmUmCmAmGmAfCmAfCmCmAmGp |
| | | | | mUmUmCmGmGmUpsmUpsmU | | smUpsmU |
| 459 | 1361 | 378 | 396 | mCpsmGpsmUmUfCmUfGfGfUmGmUmCmU | 1943 | mApsfApsmAmGmUfCmAmGmAmCmAmCmCfAmGfAmAmCmGp |
| | | | | mGmAmCmUmUmUpsmUpsmU | | smUpsmU |
| 460 | 1362 | 575 | 593 | mApsmCpsmAmUfCmUfGfCfCmCmUmAmAm | 1944 | mUpsfUpsmGmAmCfUmUmUmAmGmGmGmCfAmGfAmUmGmU |
| | | | | AmGmUmCmAmApsmUpsmU | | psmUpsmU |
| 461 | 1363 | 372 | 390 | mGpsmGpsmAmAfAmAfCfGfUmUmCmUmG | 1945 | mApsfGpsmAmCmAfCmCmAmGmAmAmCmGfUmUfUmUmCmCp |
| | | | | mGmUmGmUmCmUpsmUpsmU | | smUpsmU |
| 462 | 1364 | 387 | 405 | mGpsmUpsmCmUfGmAfCfUfUmUmCmGmG | 1946 | mUpsfUpsmUmGmGfAmCmCmGmAmAmAmGfUmCfAmGmAmC |
| | | | | mUmCmCmAmAmApsmUpsmU | | psmUpsmU |
| 463 | 1365 | 375 | 393 | mApsmApsmAmCfGmUfUfCfUmGmGmUmG | 1947 | mGpsfUpsmCmAmGfAmCmAmCmCmAmGmAfAmCfGmUmUmUp |
| | | | | mUmCmUmGmAmCpsmUpsmU | | smUpsmU |
| 464 | 1366 | 1080 | 1098 | mGpsmCpsmCmUfGmUfGfGfAmAmUmCmU | 1948 | mApsfApsmUmGmGfCmAmGmAmUmUmCmCfAmCfAmGmGmCp |
| | | | | mGmCmCmAmUmUpsmUpsmU | | smUpsmU |
| 465 | 1367 | 1039 | 1057 | mCpsmCpsmCmAfUmUfAfGfGmAmUmAmAm | 1949 | mApsfApsmGmAmCfAmUmUmAmUmCmCmUfAmAfUmGmGmG |
| | | | | UmGmUmCmUmUpsmUpsmU | | psmUpsmU |
| 466 | 1368 | 415 | 433 | mGpsmUpsmGmGfAmUfGfCfCmUmUmGmG | 1950 | mApsfApsmCmAmUfAmCmCmAmAmGmGmCfAmUfCmCmAmCp |
| | | | | mUmAmUmGmUmUpsmUpsmU | | smUpsmU |
| 467 | 1369 | 402 | 420 | mCpsmApsmAmAfGmAfCfGfAmAmGmUmCm | 1951 | mApsfUpsmCmCmAfCmGmAmCmUmUmCmGfUmCfUmUmUmG |
| | | | | GmUmGmGmAmUpsmUpsmU | | psmUpsmU |
| 468 | 1370 | 416 | 434 | mUpsmGpsmGmAfUmGfCfCfUmUmGmGmU | 1952 | mGpsfApsmAmCmAfUmAmCmCmAmAmGmGfCmAfUmCmCmAp |
| | | | | mAmUmGmUmUmCpsmUpsmU | | smUpsmU |
| 469 | 1371 | 497 | 515 | mApsmUpsmGmGfAmGfGfAfGmUmGmAmG | 1953 | vmUpsfUpsmGmUmCfAmCmUmCmAmCmUmCfCmUfCmCmAmU |
| | | | | mUmGmAmCmAmApsmUpsmU | | psmUpsmU |
| 470 | 1372 | 419 | 437 | mApsmUpsmGmCfCmUfUfGfGmUmAmUmG | 1954 | vmCpsfApsmGmGmAfAmCmAmUmAmCmCmAfAmGfGmCmAmU |
| | | | | mUmUmCmCmUmGpsmUpsmU | | psmUpsmU |
| 471 | 1373 | 377 | 395 | mApsmCpsmGmUfUmCfUfGfGmUmGmUmC | 1955 | vmApsfApsmGmUmCfAmGmAmCmAmCmCmAfGmAfAmCmGmU |
| | | | | mUmGmAmCmUmUpsmUpsmU | | psmUpsmU |
| 472 | 1374 | 386 | 404 | mUpsmGpsmUmCfUmGfAfCfUmUmUmCmG | 1956 | vmUpsfUpsmGmGmAfCmCmGmAmAmAmGmUfCmAfGmAmCmA |
| | | | | mGmUmCmCmAmApsmUpsmU | | psmUpsmU |
| 473 | 1375 | 398 | 416 | mGpsmGpsmUmCfCmAfAfAfGmAmCmGmAm | 1957 | vmApsfCpsmGmAmCfUmUmCmGmUmCmUmUfUmGfGmAmCmC |
| | | | | AmGmUmCmGmUpsmUpsmU | | psmUpsmU |
| 474 | 1376 | 502 | 520 | mGpsmGpsmAmGfUmGfAfGfUmGmAmCmA | 1958 | vmGpsfUpsmAmCmGfUmUmGmUmCmAmCmUfCmAfCmUmCmC |
| | | | | mAmCmGmUmAmCpsmUpsmU | | psmUpsmU |
| 475 | 1377 | 532 | 550 | mGpsmCpsmCmAfAmAfAfCfAmAmCmCmAm | 1959 | vmCpsfGpsmGmUmGfAmUmGmGmUmUmGmUfUmUfUmGmGm |
| | | | | UmCmAmCmCmGpsmUpsmU | | CpsmUpsmU |
| 476 | 1378 | 382 | 400 | mCpsmUpsmGmGfUmGfUfCfUmGmAmCmU | 1960 | vmApsfCpsmCmGmAfAmAmGmUmCmAmGmAfCmAfCmCmAmG |
| | | | | mUmUmCmGmGmUpsmUpsmU | | psmUpsmU |
| 477 | 1379 | 378 | 396 | mCpsmGpsmUmUfCmUfGfGfUmGmUmCmU | 1961 | vmApsfApsmAmGmUfCmAmGmAmCmAmCmCfAmGfAmAmCmG |
| | | | | mGmAmCmUmUmUpsmUpsmU | | psmUpsmU |
| 478 | 1380 | 575 | 593 | mApsmCpsmAmUfCmUfGfCfCmCmUmAmAm | 1962 | vmUpsfUpsmGmAmCfUmUmUmAmGmGmGmCfAmGfAmUmGm |
| | | | | AmGmUmCmAmApsmUpsmU | | UpsmUpsmU |
| 479 | 1381 | 372 | 390 | mGpsmGpsmAmAfAmAfCfGfUmUmCmUmG | 1963 | vmApsfGpsmAmCmAfCmCmAmGmAmAmCmGfUmUfUmUmCmC |
| | | | | mGmUmGmUmCmUpsmUpsmU | | psmUpsmU |
| 480 | 1382 | 387 | 405 | mGpsmUpsmCmUfGmAfCfUfUmUmCmGmG | 1964 | vmUpsfUpsmUmGmGfAmCmCmGmAmAmAmGfUmCfAmGmAmC |
| | | | | mUmCmCmAmAmApsmUpsmU | | psmUpsmU |
| 481 | 1383 | 375 | 393 | mApsmApsmAmCfGmUfUfCfUmGmGmUmG | 1965 | vmGpsfUpsmCmAmGfAmCmAmCmCmAmGmAfAmCfGmUmUmU |
| | | | | mUmCmUmGmAmCpsmUpsmU | | psmUpsmU |
| 482 | 1384 | 1080 | 1098 | mGpsmCpsmCmUfGmUfGfGfAmAmUmCmU | 1966 | vmApsfApsmUmGmGfCmAmGmAmUmUmCmCfAmCfAmGmGmC |
| | | | | mGmCmCmAmUmUpsmUpsmU | | psmUpsmU |
| 483 | 1385 | 1039 | 1057 | mCpsmCpsmCmAfUmUfAfGfGmAmUmAmAm | 1967 | vmApsfApsmGmAmCfAmUmUmAmUmCmCmUfAmAfUmGmGm |
| | | | | UmGmUmCmUmUpsmUpsmU | | GpsmUpsmU |
| 484 | 1386 | 415 | 433 | mGpsmUpsmGmGfAmUfGfCfCmUmUmGmG | 1968 | vmApsfApsmCmAmUfAmCmCmAmAmGmGmCfAmUfCmCmAmC |
| | | | | mUmAmUmGmUmUpsmUpsmU | | psmUpsmU |
| 485 | 1387 | 402 | 420 | mCpsmApsmAmAfGmAfCfGfAmAmGmUmCm | 1969 | vmApsfUpsmCmCmAfCmGmAmCmUmUmCmGfUmCfUmUmUmG |
| | | | | GmUmGmGmAmUpsmUpsmU | | psmUpsmU |
| 486 | 1388 | 416 | 434 | mUpsmGpsmGmAfUmGfCfCfUmUmGmGmU | 1970 | vmGpsfApsmAmCmAfUmAmCmCmAmAmGmGfCmAfUmCmCmA |
| | | | | mAmUmGmUmUmCpsmUpsmU | | psmUpsmU |
| 487 | 1389 | 107 | 127 | fUpsmCpsfCmUfCmCfGmCfGmAfCmGfCmGfC | 1971 | mApsfCpsmAfUmGfCmGfCmGfCmGfUmCfGmCfGmGfAmGfGmAp |
| | | | | mGfCmAfUmGpsfU | | smUpsmU |
| 488 | 1390 | 108 | 128 | fCpsmCpsfUmCfCmGfCmGfAmCfGmCfGmCfG | 1972 | mApsfApsmCfAmUfGmCfGmCfGmCfGmUfCmGfCmGfGmAfGmGp |
| | | | | mCfAmUfGmUpsfU | | smUpsmU |
| 489 | 1391 | 109 | 129 | fCpsmUpsfCmCfGmCfGmAfCmGfCmGfCmGfC | 1973 | mCpsfApsmAfCmAfUmGfCmGfCmGfCmGfUmCfGmCfGmGfAmGp |
| | | | | mAfUmGfUmUpsfG | | smUpsmU |
| 490 | 1392 | 110 | 130 | fUpsmCpsfCmGfCmGfAmCfGmCfGmCfGmCfA | 1974 | mApsfCpsmAfAmCfAmUfGmCfGmCfGmCfGmUfCmGfCmGfGmAp |
| | | | | mUfGmUfUmGpsfU | | smUpsmU |
| 491 | 1393 | 111 | 131 | fCpsmCpsfGmCfGmAfCmGfCmGfCmGfCmAfU | 1975 | mApsfApsmCfAmAfCmAfUmGfCmGfCmGfCmGfUmCfGmCfGmGp |
| | | | | mGfUmUfGmUpsfU | | smUpsmU |
| 492 | 1394 | 113 | 133 | fGpsmCpsfGmAfCmGfCmGfCmGfCmAfUmGfU | 1976 | mCpsfGpsmAfAmCfAmAfCmAfUmGfCmGfCmGfCmGfUmCfGmCps |
| | | | | mUfGmUfUmCpsfG | | mUpsmU |
| 493 | 1395 | 114 | 134 | fCpsmGpsfAmCfGmCfGmCfGmCfAmUfGmUfU | 1977 | mCpsfCpsmGfAmAfCmAfAmCfAmUfGmCfGmCfGmCfGmUfCmGps |
| | | | | mGfUmUfCmGpsfG | | mUpsmU |
| 494 | 1396 | 115 | 135 | fGpsmApsfCmGfCmGfCmGfCmAfUmGfUmUf | 1978 | mGpsfCpsmCfGmAfAmCfAmAfCmAfUmGfCmGfCmGfCmGfUmCps |
| | | | | GmUfUmCfGmGpsfC | | mUpsmU |
| 495 | 1397 | 213 | 233 | fUpsmCpsfUmUfGmUfGmCfGmGfAmAfGmGf | 1979 | mCpsfUpsmCfCmUfGmGfCmCfUmUfCmCfGmCfAmCfAmAfGmAps |
| | | | | CmCfAmGfGmApsfG | | mUpsmU |
| 496 | 1398 | 214 | 234 | fCpsmUpsfUmGfUmGfCmGfGmAfAmGfGmCf | 1980 | mApsfCpsmUfCmCfUmGfGmCfCmUfUmCfCmGfCmAfCmAfAmGps |
| | | | | CmAfGmGfAmGpsfU | | mUpsmU |
| 497 | 1399 | 216 | 236 | fUpsmGpsfUmGfCmGfGmAfAmGfGmCfCmAf | 1981 | mCpsfGpsmAfCmUfCmCfUmGfGmCfCmUfUmCfCmGfCmAfCmAps |
| | | | | GmGfAmGfUmCpsfG | | mUpsmU |
| 498 | 1400 | 218 | 238 | fUpsmGpsfCmGfGmAfAmGfGmCfCmAfGmGf | 1982 | mUpsfCpsmCfGmAfCmUfCmCfUmGfGmCfCmUfUmCfCmGfCmAps |
| | | | | AmGfUmCfGmGpsfA | | mUpsmU |
| 499 | 1401 | 220 | 240 | fCpsmGpsfGmAfAmGfGmCfCmAfGmGfAmGf | 1983 | mGpsfUpsmUfCmCfGmAfCmUfCmCfUmGfGmCfCmUfUmCfCmGp |
| | | | | UmCfGmGfAmApsfC | | smUpsmU |
| 500 | 1402 | 223 | 243 | fApsmApsfGmGfCmCfAmGfGmAfGmUfCmGf | 1984 | mApsfApsmUfGmUfUmCfCmGfAmCfUmCfCmUfGmGfCmCfUmUp |
| | | | | GmAfAmCfAmUpsfU | | smUpsmU |
| 501 | 1403 | 224 | 244 | fApsmGpsfGmCfCmAfGmGfAmGfUmCfGmGf | 1985 | mCpsfApsmAfUmGfUmUfCmCfGmAfCmUfCmCfUmGfGmCfCmUp |
| | | | | AmAfCmAfUmUpsfG | | smUpsmU |
| 502 | 1404 | 225 | 245 | fGpsmGpsfCmCfAmGfGmAfGmUfCmGfGmAf | 1986 | mCpsfCpsmAfAmUfGmUfUmCfCmGfAmCfUmCfCmUfGmGfCmCp |
| | | | | AmCfAmUfUmGpsfG | | smUpsmU |
| 503 | 1405 | 229 | 249 | fApsmGpsfGmAfGmUfCmGfGmAfAmCfAmUf | 1987 | mGpsfApsmUfGmCfCmAfAmUfGmUfUmCfCmGfAmCfUmCfCmUp |
| | | | | UmGfGmCfAmUpsfC | | smUpsmU |
| 504 | 1406 | 311 | 331 | fCpsmCpsfAmAfUmGfUmCfCmAfCmCfAmGfC | 1988 | mApsfGpsmAfUmGfAmGfCmUfGmGfUmGfGmAfCmAfUmUfGmG |
| | | | | mUfCmAfUmCpsfU | | psmUpsmU |
| 505 | 1407 | 312 | 332 | fCpsmApsfAmUfGmUfCmCfAmCfCmAfGmCfU | 1989 | mGpsfApsmGfAmUfGmAfGmCfUmGfGmUfGmGfAmCfAmUfUmG |
| | | | | mCfAmUfCmUpsfC | | psmUpsmU |
| 506 | 1408 | 314 | 334 | fApsmUpsfGmUfCmCfAmCfCmAfGmCfUmCfA | 1990 | mCpsfGpsmGfAmGfAmUfGmAfGmCfUmGfGmUfGmGfAmCfAmU |
| | | | | mUfCmUfCmCpsfG | | psmUpsmU |
| 507 | 1409 | 315 | 335 | fUpsmGpsfUmCfCmAfCmCfAmGfCmUfCmAfU | 1991 | mCpsfCpsmGfGmAfGmAfUmGfAmGfCmUfGmGfUmGfGmAfCmA |
| | | | | mCfUmCfCmGpsfG | | psmUpsmU |
| 508 | 1410 | 316 | 336 | fGpsmUpsfCmCfAmCfCmAfGmCfUmCfAmUfC | 1992 | mGpsfCpsmCfGmGfAmGfAmUfGmAfGmCfUmGfGmUfGmGfAmC |
| | | | | mUfCmCfGmGpsfC | | psmUpsmU |
| 509 | 1411 | 317 | 337 | fUpsmCpsfCmAfCmCfAmGfCmUfCmAfUmCfU | 1993 | mUpsfGpsmCfCmGfGmAfGmAfUmGfAmGfCmUfGmGfUmGfGmA |
| | | | | mCfCmGfGmCpsfA | | psmUpsmU |
| 510 | 1412 | 319 | 339 | fCpsmApsfCmCfAmGfCmUfCmAfUmCfUmCfC | 1994 | mUpsfUpsmUfGmCfCmGfGmAfGmAfUmGfAmGfCmUfGmGfUmG |
| | | | | mGfGmCfAmApsfA | | psmUpsmU |
| 511 | 1413 | 351 | 371 | fUpsmCpsfUmUfAmCfCmAfGmAfGmUfGmUf | 1995 | mCpsfCpsmAfUmCfAmGfAmCfAmCfUmCfUmGfGmUfAmAfGmAp |
| | | | | CmUfGmAfUmGpsfG | | smUpsmU |
| 512 | 1414 | 352 | 372 | fCpsmUpsfUmAfCmCfAmGfAmGfUmGfUmCf | 1996 | mCpsfCpsmCfAmUfCmAfGmAfCmAfCmUfCmUfGmGfUmAfAmGp |
| | | | | UmGfAmUfGmGpsfG | | smUpsmU |
| 513 | 1415 | 353 | 373 | fUpsmUpsfAmCfCmAfGmAfGmUfGmUfCmUf | 1997 | mCpsfCpsmCfCmAfUmCfAmGfAmCfAmCfUmCfUmGfGmUfAmAps |
| | | | | GmAfUmGfGmGpsfG | | mUpsmU |
| 514 | 1416 | 354 | 374 | fUpsmApsfCmCfAmGfAmGfUmGfUmCfUmGf | 1998 | mUpsfCpsmCfCmCfAmUfCmAfGmAfCmAfCmUfCmUfGmGfUmAp |
| | | | | AmUfGmGfGmGpsfA | | smUpsmU |
| 515 | 1417 | 357 | 377 | fCpsmApsfGmAfGmUfGmUfCmUfGmAfUmGf | 1999 | mUpsfUpsmUfUmCfCmCfCmAfUmCfAmGfAmCfAmCfUmCfUmGp |
| | | | | GmGfGmAfAmApsfA | | smUpsmU |
| 516 | 1418 | 358 | 378 | fApsmGpsfAmGfUmGfUmCfUmGfAmUfGmGf | 2000 | mGpsfUpsmUfUmUfCmCfCmCfAmUfCmAfGmAfCmAfCmUfCmUp |
| | | | | GmGfAmAfAmApsfC | | smUpsmU |
| 517 | 1419 | 359 | 379 | fGpsmApsfGmUfGmUfCmUfGmAfUmGfGmGf | 2001 | mCpsfGpsmUfUmUfUmCfCmCfCmAfUmCfAmGfAmCfAmCfUmCps |
| | | | | GmAfAmAfAmCpsfG | | mUpsmU |
| 518 | 1420 | 360 | 380 | fApsmGpsfUmGfUmCfUmGfAmUfGmGfGmGf | 2002 | mApsfCpsmGfUmUfUmUfCmCfCmCfAmUfCmAfGmAfCmAfCmUp |
| | | | | AmAfAmAfCmGpsfU | | smUpsmU |
| 519 | 1421 | 361 | 381 | fGpsmUpsfGmUfCmUfGmAfUmGfGmGfGmAf | 2003 | mApsfApsmCfGmUfUmUfUmCfCmCfCmAfUmCfAmGfAmCfAmCps |
| | | | | AmAfAmCfGmUpsfU | | mUpsmU |
| 520 | 1422 | 362 | 382 | fUpsmGpsfUmCfUmGfAmUfGmGfGmGfAmAf | 2004 | mGpsfApsmAfCmGfUmUfUmUfCmCfCmCfAmUfCmAfGmAfCmAp |
| | | | | AmAfCmGfUmUpsfC | | smUpsmU |
| 521 | 1423 | 363 | 383 | fGpsmUpsfCmUfGmAfUmGfGmGfGmAfAmAf | 2005 | mApsfGpsmAfAmCfGmUfUmUfUmCfCmCfCmAfUmCfAmGfAmCp |
| | | | | AmCfGmUfUmCpsfU | | smUpsmU |
| 522 | 1424 | 364 | 384 | fUpsmCpsfUmGfAmUfGmGfGmGfAmAfAmAf | 2006 | mCpsfApsmGfAmAfCmGfUmUfUmUfCmCfCmCfAmUfCmAfGmAp |
| | | | | CmGfUmUfCmUpsfG | | smUpsmU |
| 523 | 1425 | 365 | 385 | fCpsmUpsfGmAfUmGfGmGfGmAfAmAfAmCf | 2007 | mCpsfCpsmAfGmAfAmCfGmUfUmUfUmCfCmCfCmAfUmCfAmGp |
| | | | | GmUfUmCfUmGpsfG | | smUpsmU |
| 524 | 1426 | 366 | 386 | fUpsmGpsfAmUfGmGfGmGfAmAfAmAfCmGf | 2008 | mApsfCpsmCfAmGfAmAfCmGfUmUfUmUfCmCfCmCfAmUfCmAps |
| | | | | UmUfCmUfGmGpsfU | | mUpsmU |
| 525 | 1427 | 367 | 387 | fGpsmApsfUmGfGmGfGmAfAmAfAmCfGmUf | 2009 | mCpsfApsmCfCmAfGmAfAmCfGmUfUmUfUmCfCmCfCmAfUmCps |
| | | | | UmCfUmGfGmUpsfG | | mUpsmU |
| 526 | 1428 | 369 | 389 | fUpsmGpsfGmGfGmAfAmAfAmCfGmUfUmCf | 2010 | mGpsfApsmCfAmCfCmAfGmAfAmCfGmUfUmUfUmCfCmCfCmAps |
| | | | | UmGfGmUfGmUpsfC | | mUpsmU |
| 527 | 1429 | 371 | 391 | fGpsmGpsfGmAfAmAfAmCfGmUfUmCfUmGf | 2011 | mCpsfApsmGfAmCfAmCfCmAfGmAfAmCfGmUfUmUfUmCfCmCps |
| | | | | GmUfGmUfCmUpsfG | | mUpsmU |
| 528 | 1430 | 372 | 392 | fGpsmGpsfAmAfAmAfCmGfUmUfCmUfGmGf | 2012 | mUpsfCpsmAfGmAfCmAfCmCfAmGfAmAfCmGfUmUfUmUfCmCp |
| | | | | UmGfUmCfUmGpsfA | | smUpsmU |
| 529 | 1431 | 375 | 395 | fApsmApsfAmCfGmUfUmCfUmGfGmUfGmUf | 2013 | mApsfApsmGfUmCfAmGfAmCfAmCfCmAfGmAfAmCfGmUfUmUp |
| | | | | CmUfGmAfCmUpsfU | | smUpsmU |
| 530 | 1432 | 376 | 396 | fApsmApsfCmGfUmUfCmUfGmGfUmGfUmCf | 2014 | mApsfApsmAfGmUfCmAfGmAfCmAfCmCfAmGfAmAfCmGfUmUp |
| | | | | UmGfAmCfUmUpsfU | | smUpsmU |
| 531 | 1433 | 377 | 397 | fApsmCpsfGmUfUmCfUmGfGmUfGmUfCmUf | 2015 | mGpsfApsmAfAmGfUmCfAmGfAmCfAmCfCmAfGmAfAmCfGmUp |
| | | | | GmAfCmUfUmUpsfC | | smUpsmU |
| 532 | 1434 | 378 | 398 | fCpsmGpsfUmUfCmUfGmGfUmGfUmCfUmGf | 2016 | mCpsfGpsmAfAmAfGmUfCmAfGmAfCmAfCmCfAmGfAmAfCmGps |
| | | | | AmCfUmUfUmCpsfG | | mUpsmU |
| 533 | 1435 | 382 | 402 | fCpsmUpsfGmGfUmGfUmCfUmGfAmCfUmUf | 2017 | mGpsfGpsmAfCmCfGmAfAmAfGmUfCmAfGmAfCmAfCmCfAmGp |
| | | | | UmCfGmGfUmCpsfC | | smUpsmU |
| 534 | 1436 | 383 | 403 | fUpsmGpsfGmUfGmUfCmUfGmAfCmUfUmUf | 2018 | mUpsfGpsmGfAmCfCmGfAmAfAmGfUmCfAmGfAmCfAmCfCmAp |
| | | | | CmGfGmUfCmCpsfA | | smUpsmU |
| 535 | 1437 | 354 | 374 | mUpsmApsmCmCmAmGfAmGfUfGfUmCmU | 2019 | mUpsfCpsmCmCmCfAmUmCmAmGmAmCmAfCmUfCmUmGmG |
| | | | | mGmAmUmGmGmGmGpsmA | | mUmApsmUpsmU |
| 536 | 1438 | 385 | 405 | mGpsmUpsmGmUmCmUfGmAfCfUfUmUmC | 2020 | mUpsfUpsmUmGmGfAmCmCmGmAmAmAmGfUmCfAmGmAmC |
| | | | | mGmGmUmCmCmAmApsmA | | mAmCpsmUpsmU |
| 537 | 1439 | 400 | 420 | mUpsmCpsmCmAmAmAfGmAfCfGfAmAmGm | 2021 | mApsfUpsmCmCmAfCmGmAmCmUmUmCmGfUmCfUmUmUmG |
| | | | | UmCmGmUmGmGmApsmU | | mGmApsmUpsmU |
| 538 | 1440 | 409 | 429 | mGpsmApsmAmGmUmCfGmUfGfGfAmUmG | 2022 | mUpsfApsmCmCmAfAmGmGmCmAmUmCmCfAmCfGmAmCmU |
| | | | | mCmCmUmUmGmGmUpsmA | | mUmCpsmUpsmU |
| 539 | 1441 | 412 | 432 | mGpsmUpsmCmGmUmGfGmAfUfGfCmCmU | 2023 | mApsfCpsmAmUmAfCmCmAmAmGmGmCmAfUmCfCmAmCmGm |
| | | | | mUmGmGmUmAmUmGpsmU | | AmCpsmUpsmU |
| 540 | 1442 | 413 | 433 | mUpsmCpsmGmUmGmGfAmUfGfCfCmUmU | 2024 | mApsfApsmCmAmUfAmCmCmAmAmGmGmCfAmUfCmCmAmCm |
| | | | | mGmGmUmAmUmGmUpsmU | | GmApsmUpsmU |
| 541 | 1443 | 421 | 441 | mGpsmCpsmCmUmUmGfGmUfAfUfGmUmU | 2025 | mGpsfApsmAmGmCfAmGmGmAmAmCmAmUfAmCfCmAmAmG |
| | | | | mCmCmUmGmCmUmUpsmC | | mGmCpsmUpsmU |
| 542 | 1444 | 530 | 550 | mApsmUpsmGmCmCmAfAmAfAfCfAmAmCm | 2026 | mCpsfGpsmGmUmGfAmUmGmGmUmUmGmUfUmUfUmGmGm |
| | | | | CmAmUmCmAmCmCpsmG | | CmAmUpsmUpsmU |
| 543 | 1445 | 533 | 553 | mCpsmCpsmAmAmAmAfCmAfAfCfCmAmUm | 2027 | mApsfCpsmAmCmGfGmUmGmAmUmGmGmUfUmGfUmUmUmU |
| | | | | CmAmCmCmGmUmGpsmU | | mGmGpsmUpsmU |
| 544 | 1446 | 573 | 593 | mCpsmGpsmAmCmAmUfCmUfGfCfCmCmUm | 2028 | mUpsfUpsmGmAmCfUmUmUmAmGmGmGmCfAmGfAmUmGmU |
| | | | | AmAmAmGmUmCmApsmA | | mCmGpsmUpsmU |
| 545 | 1447 | 619 | 639 | mApsmUpsmCmAmCmCfAmAfGfCfUmCmAm | 2029 | mGpsfCpsmGmUmAfGmAmCmUmGmAmGmCfUmUfGmGmUmG |
| | | | | GmUmCmUmAmCmGpsmC | | mAmUpsmUpsmU |
| 546 | 1448 | 1039 | 1059 | mCpsmCpsmCmAmUmUfAmGfGfAfUmAmAm | 2030 | mApsfUpsmAmAmGfAmCmAmUmUmAmUmCfCmUfAmAmUmG |
| | | | | UmGmUmCmUmUmApsmU | | mGmGpsmUpsmU |
| 547 | 1449 | 354 | 374 | mUpsmApsmCmCmAmGfAmGfUfGfUmCmU | 2031 | vmUpsfCpsmCmCmCfAmUmCmAmGmAmCmAfCmUfCmUmGmG |
| | | | | mGmAmUmGmGmGmGpsmA | | mUmApsmUpsmU |
| 548 | 1450 | 385 | 405 | mGpsmUpsmGmUmCmUfGmAfCfUfUmUmC | 2032 | vmUpsfUpsmUmGmGfAmCmCmGmAmAmAmGfUmCfAmGmAmC |
| | | | | mGmGmUmCmCmAmApsmA | | mAmCpsmUpsmU |
| 549 | 1451 | 400 | 420 | mUpsmCpsmCmAmAmAfGmAfCfGfAmAmGm | 2033 | vmApsfUpsmCmCmAfCmGmAmCmUmUmCmGfUmCfUmUmUmG |
| | | | | UmCmGmUmGmGmApsmU | | mGmApsmUpsmU |
| 550 | 1452 | 409 | 429 | mGpsmApsmAmGmUmCfGmUfGfGfAmUmG | 2034 | vmUpsfApsmCmCmAfAmGmGmCmAmUmCmCfAmCfGmAmCmU |
| | | | | mCmCmUmUmGmGmUpsmA | | mUmCpsmUpsmU |
| 551 | 1453 | 412 | 432 | mGpsmUpsmCmGmUmGfGmAfUfGfCmCmU | 2035 | vmApsfCpsmAmUmAfCmCmAmAmGmGmCmAfUmCfCmAmCmG |
| | | | | mUmGmGmUmAmUmGpsmU | | mAmCpsmUpsmU |
| 552 | 1454 | 413 | 433 | mUpsmCpsmGmUmGmGfAmUfGfCfCmUmU | 2036 | vmApsfApsmCmAmUfAmCmCmAmAmGmGmCfAmUfCmCmAmC |
| | | | | mGmGmUmAmUmGmUpsmU | | mGmApsmUpsmU |
| 553 | 1455 | 421 | 441 | mGpsmCpsmCmUmUmGfGmUfAfUfGmUmU | 2037 | vmGpsfApsmAmGmCfAmGmGmAmAmCmAmUfAmCfCmAmAmG |
| | | | | mCmCmUmGmCmUmUpsmC | | mGmCpsmUpsmU |
| 554 | 1456 | 530 | 550 | mApsmUpsmGmCmCmAfAmAfAfCfAmAmCm | 2038 | vmCpsfGpsmGmUmGfAmUmGmGmUmUmGmUfUmUfUmGmGm |
| | | | | CmAmUmCmAmCmCpsmG | | CmAmUpsmUpsmU |
| 555 | 1457 | 533 | 553 | mCpsmCpsmAmAmAmAfCmAfAfCfCmAmUm | 2039 | vmApsfCpsmAmCmGfGmUmGmAmUmGmGmUfUmGfUmUmUm |
| | | | | CmAmCmCmGmUmGpsmU | | UmGmGpsmUpsmU |
| 556 | 1458 | 573 | 593 | mCpsmGpsmAmCmAmUfCmUfGfCfCmCmUm | 2040 | vmUpsfUpsmGmAmCfUmUmUmAmGmGmGmCfAmGfAmUmGm |
| | | | | AmAmAmGmUmCmApsmA | | UmCmGpsmUpsmU |
| 557 | 1459 | 619 | 639 | mApsmUpsmCmAmCmCfAmAfGfCfUmCmAm | 2041 | vmGpsfCpsmGmUmAfGmAmCmUmGmAmGmCfUmUfGmGmUm |
| | | | | GmUmCmUmAmCmGpsmC | | GmAmUpsmUpsmU |
| 558 | 1460 | 1039 | 1059 | mCpsmCpsmCmAmUmUfAmGfGfAfUmAmAm | 2042 | vmApsfUpsmAmAmGfAmCmAmUmUmAmUmCfCmUfAmAmUm |
| | | | | UmGmUmCmUmUmApsmU | | GmGmGpsmUpsmU |
| 559 | 1461 | 354 | 374 | mUpsmApsmCmCfAmGmAmGfUfGfUmCmUf | 2043 | mUpsfCpsmCmCmCmAmUmCmAmGmAmCmAfCmUmCmUmGm |
| | | | | GmAmUmGmGfGmGpsmA | | GmUmApsmUpsmU |
| 560 | 1462 | 385 | 405 | mGpsmUpsmGmUfCmUmGmAfCfUfUmUmCf | 2044 | mUpsfUpsmUmGmGmAmCmCmGmAmAmAmGfUmCmAmGmAm |
| | | | | GmGmUmCmCfAmApsmA | | CmAmCpsmUpsmU |
| 561 | 1463 | 400 | 420 | mUpsmCpsmCmAfAmAmGmAfCfGfAmAmGfU | 2045 | mApsfUpsmCmCmAmCmGmAmCmUmUmCmGfUmCmUmUmUm |
| | | | | mCmGmUmGfGmApsmU | | GmGmApsmUpsmU |
| 562 | 1464 | 409 | 429 | mGpsmApsmAmGfUmCmGmUfGfGfAmUmGf | 2046 | mUpsfApsmCmCmAmAmGmGmCmAmUmCmCfAmCmGmAmCm |
| | | | | CmCmUmUmGfGmUpsmA | | UmUmCpsmUpsmU |
| 563 | 1465 | 412 | 432 | mGpsmUpsmCmGfUmGmGmAfUfGfCmCmUf | 2047 | mApsfCpsmAmUmAmCmCmAmAmGmGmCmAfUmCmCmAmCm |
| | | | | UmGmGmUmAfUmGpsmU | | GmAmCpsmUpsmU |
| 564 | 1466 | 413 | 433 | mUpsmCpsmGmUfGmGmAmUfGfCfCmUmUf | 2048 | mApsfApsmCmAmUmAmCmCmAmAmGmGmCfAmUmCmCmAm |
| | | | | GmGmUmAmUfGmUpsmU | | CmGmApsmUpsmU |
| 565 | 1467 | 421 | 441 | mGpsmCpsmCmUfUmGmGmUfAfUfGmUmUf | 2049 | mGpsfApsmAmGmCmAmGmGmAmAmCmAmUfAmCmCmAmAm |
| | | | | CmCmUmGmCfUmUpsmC | | GmGmCpsmUpsmU |
| 566 | 1468 | 530 | 550 | mApsmUpsmGmCfCmAmAmAfAfCfAmAmCfC | 2050 | mCpsfGpsmGmUmGmAmUmGmGmUmUmGmUfUmUmUmGmG |
| | | | | mAmUmCmAfCmCpsmG | | mCmAmUpsmUpsmU |
| 567 | 1469 | 533 | 553 | mCpsmCpsmAmAfAmAmCmAfAfCfCmAmUfC | 2051 | mApsfCpsmAmCmGmGmUmGmAmUmGmGmUfUmGmUmUmU |
| | | | | mAmCmCmGfUmGpsmU | | mUmGmGpsmUpsmU |
| 568 | 1470 | 573 | 593 | mCpsmGpsmAmCfAmUmCmUfGfCfCmCmUfA | 2052 | mUpsfUpsmGmAmCmUmUmUmAmGmGmGmCfAmGmAmUmG |
| | | | | mAmAmGmUfCmApsmA | | mUmCmGpsmUpsmU |
| 569 | 1471 | 619 | 639 | mApsmUpsmCmAfCmCmAmAfGfCfUmCmAfG | 2053 | mGpsfCpsmGmUmAmGmAmCmUmGmAmGmCfUmUmGmGmU |
| | | | | mUmCmUmAfCmGpsmC | | mGmAmUpsmUpsmU |
| 570 | 1472 | 1039 | 1059 | mCpsmCpsmCmAfUmUmAmGfGfAfUmAmAf | 2054 | mApsfUpsmAmAmGmAmCmAmUmUmAmUmCfCmUmAmAmUm |
| | | | | UmGmUmCmUfUmApsmU | | GmGmGpsmUpsmU |
| 571 | 1473 | 354 | 374 | mUpsmApsmCmCfAmGmAmGfUfGfUmCmUf | 2055 | vmUpsfCpsmCmCmCmAmUmCmAmGmAmCmAfCmUmCmUmG |
| | | | | GmAmUmGmGfGmGpsmA | | mGmUmApsmUpsmU |
| 572 | 1474 | 385 | 405 | mGpsmUpsmGmUfCmUmGmAfCfUfUmUmCf | 2056 | vmUpsfUpsmUmGmGmAmCmCmGmAmAmAmGfUmCmAmGmA |
| | | | | GmGmUmCmCfAmApsmA | | mCmAmCpsmUpsmU |
| 573 | 1475 | 400 | 420 | mUpsmCpsmCmAfAmAmGmAfCfGfAmAmGfU | 2057 | vmApsfUpsmCmCmAmCmGmAmCmUmUmCmGfUmCmUmUmU |
| | | | | mCmGmUmGfGmApsmU | | mGmGmApsmUpsmU |
| 574 | 1476 | 409 | 429 | mGpsmApsmAmGfUmCmGmUfGfGfAmUmGf | 2058 | vmUpsfApsmCmCmAmAmGmGmCmAmUmCmCfAmCmGmAmC |
| | | | | CmCmUmUmGfGmUpsmA | | mUmUmCpsmUpsmU |
| 575 | 1477 | 412 | 432 | mGpsmUpsmCmGfUmGmGmAfUfGfCmCmUf | 2059 | vmApsfCpsmAmUmAmCmCmAmAmGmGmCmAfUmCmCmAmCm |
| | | | | UmGmGmUmAfUmGpsmU | | GmAmCpsmUpsmU |
| 576 | 1478 | 413 | 433 | mUpsmCpsmGmUfGmGmAmUfGfCfCmUmUf | 2060 | vmApsfApsmCmAmUmAmCmCmAmAmGmGmCfAmUmCmCmA |
| | | | | GmGmUmAmUfGmUpsmU | | mCmGmApsmUpsmU |
| 577 | 1479 | 421 | 441 | mGpsmCpsmCmUfUmGmGmUfAfUfGmUmUf | 2061 | vmGpsfApsmAmGmCmAmGmGmAmAmCmAmUfAmCmCmAmA |
| | | | | CmCmUmGmCfUmUpsmC | | mGmGmCpsmUpsmU |
| 578 | 1480 | 530 | 550 | mApsmUpsmGmCfCmAmAmAfAfCfAmAmCfC | 2062 | vmCpsfGpsmGmUmGmAmUmGmGmUmUmGmUfUmUmUmGm |
| | | | | mAmUmCmAfCmCpsmG | | GmCmAmUpsmUpsmU |
| 579 | 1481 | 533 | 553 | mCpsmCpsmAmAfAmAmCmAfAfCfCmAmUfC | 2063 | vmApsfCpsmAmCmGmGmUmGmAmUmGmGmUfUmGmUmUmU |
| | | | | mAmCmCmGfUmGpsmU | | mUmGmGpsmUpsmU |
| 580 | 1482 | 573 | 593 | mCpsmGpsmAmCfAmUmCmUfGfCfCmCmUfA | 2064 | vmUpsfUpsmGmAmCmUmUmUmAmGmGmGmCfAmGmAmUmG |
| | | | | mAmAmGmUfCmApsmA | | mUmCmGpsmUpsmU |
| 581 | 1483 | 619 | 639 | mApsmUpsmCmAfCmCmAmAfGfCfUmCmAfG | 2065 | vmGpsfCpsmGmUmAmGmAmCmUmGmAmGmCfUmUmGmGmU |
| | | | | mUmCmUmAfCmGpsmC | | mGmAmUpsmUpsmU |
| 582 | 1484 | 1039 | 1059 | mCpsmCpsmCmAfUmUmAmGfGfAfUmAmAf | 2066 | vmApsfUpsmAmAmGmAmCmAmUmUmAmUmCfCmUmAmAmU |
| | | | | UmGmUmCmUfUmApsmU | | mGmGmGpsmUpsmU |
| 583 | 2148 | | | mApsmUpsmGmCfCmUfUfGfGmUmAmUmG | 1936 | mCpsfApsmGmGmAfAmCmAmUmAmCmCmAfAmGfGmCmAmUp |
| | | | | mUmUmCmCmUmG-GalNAc4-ps-GalNAc4- | | smUpsmU |
| | | | | ps-GalNAc4 | | |
| 584 | 2148 | | | mApsmUpsmGmCfCmUfUfGfGmUmAmUmG | 2188 | vmCpsfApsmGmGmAfAmCmAmUmAmCmCmAfAmGfGmCmAmU |
| | | | | mUmUmCmCmUmG-GalNAc4-ps-GalNAc4- | | psmUpsmU |
| | | | | ps-GalNAc4 | | |
| 585 | 2293 | | | mApsmUpsmGmGfAmGfGfAfGmUmGmAm | 2317 | mUpsfUpsmGmUmCfAmCmUmCmAmCmUmCfCmUfCmCmAmUp |
| | | | | GmUmGmAmCmAmA-GalNAc4-ps-GalNAc4- | | smUpsmU |
| | | | | ps-GalNAc4 | | |
| 586 | 2293 | | | mApsmUpsmGmGfAmGfGfAfGmUmGmAm | 1953 | vmUpsfUpsmGmUmCfAmCmUmCmAmCmUmCfCmUfCmCmAmU |
| | | | | GmUmGmAmCmAmA-GalNAc4-ps-GalNAc4- | | psmUpsmU |
| | | | | ps-GalNAc4 | | |
| 587 | 2354 | | | mCpsmGpsmUmUfCmUfGfGfUmGmUmCmU | 1943 | mApsfApsmAmGmUfCmAmGmAmCmAmCmCfAmGfAmAmCmGp |
| | | | | mGmAmCmUmUmU-GalNAc4-ps-GalNAc4- | | smUpsmU |
| | | | | ps-GalNAc4 | | |
| 588 | 2354 | | | mCpsmGpsmUmUfCmUfGfGfUmGmUmCmU | 1961 | vmApsfApsmAmGmUfCmAmGmAmCmAmCmCfAmGfAmAmCmG |
| | | | | mGmAmCmUmUmU-GalNAc4-ps-GalNAc4- | | psmUpsmU |
| | | | | ps-GalNAc4 | | |
| 589 | 2297 | | | mGpsmUpsmGmUmCmUfGmAfCfUfUmUmC | 2321 | mUpsfUpsmUmGmGfAmCmCmGmAmAmAmGfUmCfAmGmAmC |
| | | | | mGmGmUmCmCmAmAmA-GalNAc4-ps- | | mAmCpsmUpsmU |
| | | | | GalNAc4-ps-GalNAc4 | | |
| 590 | 2173 | | | mGpsmUpsmGmUmCmUfGmAfCfUfUmUmC | 2213 | vmUpsfUpsmUmGmGfAmCmCmGmAmAmAmGfUmCfAmGmAmC |
| | | | | mGmGmUmCmCmAmAmA-GalNAc4-ps- | | mAmCpsmUpsmU |
| | | | | GalNAc4-ps-GalNAc4 | | |
| 591 | 2186 | | | mUpsmCpsmGmUmGmGfAmUfGfCfCmUmU | 2024 | mApsfApsmCmAmUfAmCmCmAmAmGmGmCfAmUfCmCmAmCm |
| | | | | mGmGmUmAmUmGmUmU-GalNAc4-ps- | | GmApsmUpsmU |
| | | | | GalNAc4-ps-GalNAc4 | | |
| 592 | 2292 | | | mUpsmCpsmGmUmGmGfAmUfGfCfCmUmU | 2316 | vmApsfApsmCmAmUfAmCmCmAmAmGmGmCfAmUfCmCmAmC |
| | | | | mGmGmUmAmUmGmUmU-GalNAc4-ps- | | mGmApsmUpsmU |
| | | | | GalNAc4-ps-GalNAc4 | | |
| 593 | 2294 | | | mApsmUpsmGmCmCmAfAmAfAfCfAmAmC | 2318 | mCpsfGpsmGmUmGfAmUmGmGmUmUmGmUfUmUfUmGmGm |
| | | | | mCmAmUmCmAmCmCmG-GalNAc4-ps- | | CmAmUpsmUpsmU |
| | | | | GalNAc4-ps-GalNAc4 | | |
| 594 | 2300 | | | mApsmUpsmGmCmCmAfAmAfAfCfAmAmC | 2324 | vmCpsfGpsmGmUmGfAmUmGmGmUmUmGmUfUmUfUmGmGm |
| | | | | mCmAmUmCmAmCmCmG-GalNAc4-ps- | | CmAmUpsmUpsmU |
| | | | | GalNAc4-ps-GalNAc4 | | |
| 595 | 2301 | | | mGpsmApsmAmGmUmCfGmUfGfGfAmUmG | 2022 | mUpsfApsmCmCmAfAmGmGmCmAmUmCmCfAmCfGmAmCmU |
| | | | | mCmCmUmUmGmGmUmA-GalNAc4-ps- | | mUmCpsmUpsmU |
| | | | | GalNAc4-ps-GalNAc4 | | |
| 596 | 2301 | | | mGpsmApsmAmGmUmCfGmUfGfGfAmUmG | 2325 | vmUpsfApsmCmCmAfAmGmGmCmAmUmCmCfAmCfGmAmCmU |
| | | | | mCmCmUmUmGmGmUmA-GalNAc4-ps- | | mUmCpsmUpsmU |
| | | | | GalNAc4-ps-GalNAc4 | | |
| 597 | 2295 | | | mGpsmApsmAmGfUmCmGmUfGfGfAmUmG | 2319 | mUpsfApsmCmCmAmAmGmGmCmAmUmCmCfAmCmGmAmCm |
| | | | | fCmCmUmUmGfGmUmA-GalNAc4-ps- | | UmUmCpsmUpsmU |
| | | | | GalNAc4-ps-GalNAc4 | | |
| 598 | 2296 | | | mGpsmApsmAmGfUmCmGmUfGfGfAmUmG | 2320 | vmUpsfApsmCmCmAmAmGmGmCmAmUmCmCfAmCmGmAmC |
| | | | | fCmCmUmUmGfGmUmA-GalNAc4-ps- | | mUmUmCpsmUpsmU |
| | | | | GalNAc4-ps-GalNAc4 | | |
| 599 | 2177 | | | mUpsmCpsmCmAmAmAfGmAfCfGfAmAmG | 2021 | mApsfUpsmCmCmAfCmGmAmCmUmUmCmGfUmCfUmUmUmG |
| | | | | mUmCmGmUmGmGmAmU-GalNAc4-ps- | | mGmApsmUpsmU |
| | | | | GalNAc4-ps-GalNAc4 | | |
| 600 | 2177 | | | mUpsmCpsmCmAmAmAfGmAfCfGfAmAmG | 2033 | vmApsfUpsmCmCmAfCmGmAmCmUmUmCmGfUmCfUmUmUmG |
| | | | | mUmCmGmUmGmGmAmU-GalNAc4-ps- | | mGmApsmUpsmU |
| | | | | GalNAc4-ps-GalNAc4 | | |
| 601 | 2291 | | | mGpsmUpsmCmGmUmGfGmAfUfGfCmCmU | 2023 | mApsfCpsmAmUmAfCmCmAmAmGmGmCmAfUmCfCmAmCmGm |
| | | | | mUmGmGmUmAmUmGmU-GalNAc4-ps- | | AmCpsmUpsmU |
| | | | | GalNAc4-ps-GalNAc4 | | |
| 602 | 2291 | | | mGpsmUpsmCmGmUmGfGmAfUfGfCmCmU | 2035 | vmApsfCpsmAmUmAfCmCmAmAmGmGmCmAfUmCfCmAmCmG |
| | | | | mUmGmGmUmAmUmGmU-GalNAc4-ps- | | mAmCpsmUpsmU |
| | | | | GalNAc4-ps-GalNAc4 | | |
| 603 | 2355 | | | mGpsmCpsmCmUmUmGfGmUfAfUfGmUm | 2025 | mGpsfApsmAmGmCfAmGmGmAmAmCmAmUfAmCfCmAmAmG |
| | | | | UmCmCmUmGmCmUmUmC-GalNAc4-ps- | | mGmCpsmUpsmU |
| | | | | GalNAc4-ps-GalNAc4 | | |
| 604 | 2355 | | | mGpsmCpsmCmUmUmGfGmUfAfUfGmUm | 2037 | vmGpsfApsmAmGmCfAmGmGmAmAmCmAmUfAmCfCmAmAmG |
| | | | | UmCmCmUmGmCmUmUmC-GalNAc4-ps- | | mGmCpsmUpsmU |
| | | | | GalNAc4-ps-GalNAc4 | | |
| 605 | 2356 | | | mGpsmCpsmCmUfUmGmGmUfAfUfGmUm | 2049 | mGpsfApsmAmGmCmAmGmGmAmAmCmAmUfAmCmCmAmAm |
| | | | | UfCmCmUmGmCfUmUmC-GalNAc4-ps- | | GmGmCpsmUpsmU |
| | | | | GalNAc4-ps-GalNAc4 | | |
| 606 | 2356 | | | mGpsmCpsmCmUfUmGmGmUfAfUfGmUm | 2061 | vmGpsfApsmAmGmCmAmGmGmAmAmCmAmUfAmCmCmAmA |
| | | | | UfCmCmUmGmCfUmUmC-GalNAc4-ps- | | mGmGmCpsmUpsmU |
| | | | | GalNAc4-ps-GalNAc4 | | |
| 607 | 2357 | | | mGpsmUpsmCmGfUmGmGmAfUfGfCmCmU | 2059 | vmApsfCpsmAmUmAmCmCmAmAmGmGmCmAfUmCmCmAmCm |
| | | | | fUmGmGmUmAfUmGmU-GalNAc4-ps- | | GmAmCpsmUpsmU |
| | | | | GalNAc4-ps-GalNAc4 | | |
| 608 | 2358 | | | mApsmUpsmGmCfCmAmAmAfAfCfAmAmCf | 2062 | vmCpsfGpsmGmUmGmAmUmGmGmUmUmGmUfUmUmUmGm |
| | | | | CmAmUmCmAfCmCmG-GalNAc4-ps- | | GmCmAmUpsmUpsmU |
| | | | | GalNAc4-ps-GalNAc4 | | |
| 609 | 2148 | | | mApsmUpsmGmCfCmUfUfGfGmUmAmUmG | 2188 | vmCpsfApsmGmGmAfAmCmAmUmAmCmCmAfAmGfGmCmAmU |
| | | | | mUmUmCmCmUmG- | | psmUpsmU |
| | | | | GalNAc 4psGalNAc4psGalNAc4 | | |
| 610 | 2149 | | | mApsmUpsmGmCfCmUfUfGfGmUmAmUmG | 2189 | mApsfApsmGmGmAfAmCmAmUmAmCmCmAfAmGfGmCmAmUp |
| | | | | mUmUmCmCmUmG-p-(ps)2-GalNAc4 | | smUpsmU |
| 611 | 2150 | | | mApsmUpsmGmCfCmUfUfGfGmUmAmUmG | 2190 | vmApsfApsmGmGmAfAmCmAmUmAmCmCmAfAmGfGmCmAmU |
| | | | | mUmUmCmCmUmG- | | psmUpsmU |
| | | | | GalNAc4psGalNAc4psGalNAc4 | | |
| 612 | 2151 | | | mApsmUpsmGmCfCmUfUfGfGmUmAmUmG | 2191 | mUpsfApsmGmGmAfAmCmAmUmAmCmCmAfAmGfGmCmAmUp |
| | | | | mUmUmCmCmUmG-p-(ps)2-GalNAc4 | | smUpsmU |
| 613 | 2152 | | | mApsmUpsmGmCfCmUfUfGfGmUmAmUmG | 2192 | vmUpsfApsmGmGmAfAmCmAmUmAmCmCmAfAmGfGmCmAmU |
| | | | | mUmUmCmCmUmG-p-(ps)2-GalNAc4 | | psmUpsmU |
| 614 | 2153 | | | mApsmUpsmGmCfCmUfUfGfGmUmAmUmG | 2193 | mCpsfApsmGmGmAfAmCmAmUmAmCmCmAfAmGfGmCmAmUp |
| | | | | mUmUmCmCmUmG-p-(ps)2-GalNAc4 | | smCpsmC |
| 615 | 2154 | | | mApsmUpsmGmCfCmUfUfGfGmUmAmUmG | 2194 | mApsfApsmGmGmAfAmCmAmUmAmCmCmAfAmGfGmCmAmUp |
| | | | | mUmUmCmCmUmG-p-(ps)2-GalNAc4 | | smCpsmC |
| 616 | 2155 | | | mApsmUpsmGmCmCmUfUmGfGfUfAmUmG | 2195 | mApsfGpsmCmAmGfGmAmAmCmAmUmAmCfCmAfAmGmGmC |
| | | | | mUmUmCmCmUmGmCmU-p-(ps)2-GalNAc4 | | mAmUpsmCpsmC |
| 617 | 2156 | | | mGpsmGpsmAmUmGmCfCmUfUfGfGmUmA | 2196 | mApsfApsmGmGmAfAmCmAmUmAmCmCmAfAmGfGmCmAmU |
| | | | | mUmGmUmUmCmCmUmG-p-(ps)2- | | mCmCpsmApsmC |
| | | | | GalNAc4 | | |
| 618 | 2157 | | | mApsmUpsmGmCfCmUfUfGfAmUmAmUmG | 2197 | vmUpsfApsmGmGmAfAmCmAmUmAmUmCmAfAmGfGmCmAmU |
| | | | | mUmUmCmCmUmG-p-(ps)2-GalNAc4 | | psmUpsmU |
| 619 | 2158 | | | mApsmUpsmGmCfCmUfUfGfGmUmUmUm | 2198 | vmUpsfApsmGmGmAfAmCmAmAmAmCmCmAfAmGfGmCmAmU |
| | | | | GmUmUmCmCmUmG-p-(ps)2-GalNAc4 | | psmUpsmU |
| 620 | 2159 | | | mApsmUpsmGmCfCmUfUfGfGmUmUmUm | 2199 | d2vmApsfApsmGmGmAfAmCmAmAmAmCmCmAfAmGfGmCmA |
| | | | | GmUmUmCmCmUmG-p-(ps)2-GalNAc4 | | mUpsmCpsmC |
| 621 | 2160 | | | mApsmUpsmGmCfCmUfUfGfGmUmAmUmG | 2200 | mCpsfApsmGmGfAmAmCfAmUmAmCmCmAfAmGmGfCmAmUps |
| | | | | mUmUmCmCmUmG- | | mUpsmU |
| | | | | GalNAc4psGalNAc4psGalNAc4 | | |
| 622 | 2161 | | | GalNAc4psGalNAc4psGalNAc4- | 2201 | mCpsfApsmGmGmAfAmCmAmUmAmCmCmAfAmGfGmCmAmUp |
| | | | | mAmUmGmCfCmUfUfGfGmUmAmUmGmU | | smUpsmU |
| | | | | mUmCmCmUmG- | | |
| | | | | GalNAc4psGalNAc4psGalNAc4 | | |
| 623 | 2162 | | | mApsmUpsmGmCfCmUfUfGfGmUmAmUmG | 2202 | mCpsfApsmGmGmAfAmCmAmUmAmCmCmAfAmGfGmCmAmUp |
| | | | | mUmUmCmCmUmG- | | smUpsmU |
| | | | | GalNAc4psGalNAc4psGalNAc4psGalNac4 | | |
| 624 | 2163 | | | mApsmUpsmGmCf2PmUfUfGfGmUmAmUm | 2203 | mCpsfApsmGmGmAfAmCmAmUmAmCmCmAfAmGfGmCmAmUp |
| | | | | GmUmUmCmCmUmG- | | smUpsmU |
| | | | | GalNAc4psGalNAc4psGalNAc4 | | |
| 625 | 2164 | | | mApsmUpsmGmCfCmUfUfGfGmUmAmUmG | 2204 | mCpsfApsmGmGmAfAmCmAmUmAmCmCmAfAmGfGmCmAmUp |
| | | | | mUmUmCmun34CmUmG- | | smUpsmU |
| | | | | GalNAc4psGalNAc4psGalNAc4 | | |
| 626 | 2165 | | | mApsmUpsmGmCfCmUfUfGfGmUmAmUmG | 2205 | d2vmApsfApsmGmGmAfAmCmAmUmAmCmCmAfAmGfGmCmA |
| | | | | mUmUmCmCmUmG- | | mUpsmCpsmC |
| | | | | GalNAc4psGalNAc4psGalNAc4 | | |
| 627 | 2166 | | | mApsmUpsmGmCf2PmUfUfGfGmUmAmUm | 2206 | mApsfApsmGmGmAfAmCmAmUmAmCmCmAfAmGfGmCmAmUp |
| | | | | GmUmUmCmCmUmG- | | smCpsmC |
| | | | | GalNAc4psGalNAc4psGalNAc4 | | |
| 628 | 2167 | | | mApsmUpsmGmCf2PmUfUfGfGmUmAmUm | 2207 | d2vmApsfApsmGmGmAfAmCmAmUmAmCmCmAfAmGfGmCmA |
| | | | | GmUmUmCmCmUmG- | | mUpsmCpsmC |
| | | | | GalNAc4psGalNAc4psGalNAc4 | | |
| 629 | 2168 | | | mApsmUpsmGmCfCmUfUfGfGmUmAmUmG | 2208 | d2vmApsfApsmGmGmAfAmCmAmUmAmCmCmAfAmGfGmCmA |
| | | | | mUmUmCmun34CmUmG-p-(ps)2-GalNAc4 | | mUpsmCpsmC |
| 630 | 2169 | | | GalNAc4-(ps)2-p- | 2209 | mApsfApsmGmGmAfAmCmAmUmAmCmCmAfAmGfGmCmAmUp |
| | | | | mAmUmGmCfCmUfUfGfGmUmAmUmGmU | | smCpsmC |
| | | | | mUmCmCmUmG-p-(ps)2-GalNAc4 | | |
| 631 | 2170 | | | mApsmUpsmGmCfCmUfUfGfGmUmAmUmG | 2210 | c20- |
| | | | | mUmUmCmCmUmG-p-(ps)2-GalNAc4 | | 4hUpsfApsmGmGmAfAmCmAmUmAmCmCmAfAmGfGmCmAmU |
| | | | | | | psmCpsmC |
| 632 | 2171 | | | mGpsmGpsmAmUmGmCfCmUfUfGfGmUmA | 2211 | mApsfApsmGmGmAfAmCmAmUmAmCmCmAfAmGfGmCmAmU |
| | | | | mUmGmUmUmCmCmUmG-p-(ps)2- | | mCmCpsmApsmC |
| | | | | GalNAc4 | | |
| 633 | 2172 | | | mApsmUpsmGmCfCmUfUfGfGmUmUmUm | 2212 | vmApsfApsmGmGmAfAmCmAmAmAmCmCmAfAmGfGmCmAmU |
| | | | | GmUmUmCmCmUmG-p-(ps)2-GalNAc4 | | psmCpsmC |
| 634 | 2173 | | | mGpsmUpsmGmUmCmUfGmAfCfUfUmUmC | 2213 | vmUpsfUpsmUmGmGfAmCmCmGmAmAmAmGfUmCfAmGmAmC |
| | | | | mGmGmUmCmCmAmAmA-p-(ps)2-GalNAc4 | | mAmCpsmUpsmU |
| 635 | 2174 | | | mGpsmUpsmGmUmCmUfGmAfCfUfUmUmC | 2214 | vmUpsfUpsmUmGmGfAmCmCmGmAmAmAmGfUmCfAmGmAmC |
| | | | | mGmGmUmCmCmAmAmA-p-(ps)2-GalNAc4 | | mAmCpsmCpsmA |
| 636 | 2175 | | | mGpsmUpsmGmUmCmUfGmAfCfUfUmUmC | 2215 | vmApsfUpsmUmGmGfAmCmCmGmAmAmAmGfUmCfAmGmAmC |
| | | | | mGmGmUmCmCmAmAmA-p-(ps)2-GalNAc4 | | mAmCpsmCpsmA |
| 637 | 2176 | | | mGpsmUpsmGmUmCmUfGmAfCfUfUmUmC | 2216 | mApsfUpsmUmGmGfAmCmCmGmAmAmAmGfUmCfAmGmAmC |
| | | | | mGmGmUmCmCmAmAmA-p-(ps)2-GalNAc4 | | mAmCpsmCpsmA |
| 638 | 2177 | | | mUpsmCpsmCmAmAmAfGmAfCfGfAmAmG | 2217 | vmApsfUpsmCmCmAfCmGmAmCmUmUmCmGfUmCfUmUmUmG |
| | | | | mUmCmGmUmGmGmAmU-p-(ps)2- | | mGmApsmCpsmC |
| | | | | GalNAc4 | | |
| 639 | 2178 | | | mUpsmCpsmCmAmAmAfGmAfCfGfAmAmG | 2218 | vmUpsfUpsmCmCmAfCmGmAmCmUmUmCmGfUmCfUmUmUm |
| | | | | mUmCmGmUmGmGmAmU-p-(ps)2- | | GmGmApsmCpsmC |
| | | | | GalNAc4 | | |
| 640 | 2179 | | | mUpsmCpsmCmAmAmAfGmAfCfGfAmAmG | 2219 | mUpsfUpsmCmCmAfCmGmAmCmUmUmCmGfUmCfUmUmUmG |
| | | | | mUmCmGmUmGmGmAmU-p-(ps)2- | | mGmApsmCpsmC |
| | | | | GalNAc4 | | |
| 641 | 2180 | | | mApsmUpsmGmCmCmAfAmAfAfCfAmAmC | 2220 | vmApsfGpsmGmUmGfAmUmGmGmUmUmGmUfUmUfUmGmG |
| | | | | mCmAmUmCmAmCmCmG-p-(ps)2-GalNAc4 | | mCmAmUpsmUpsmU |
| 642 | 2181 | | | mApsmUpsmGmCmCmAfAmAfAfCfAmAmC | 2221 | vmApsfGpsmGmUmGfAmUmGmGmUmUmGmUfUmUfUmGmG |
| | | | | mCmAmUmCmAmCmCmG-p-(ps)2-GalNAc4 | | mCmAmUpsmCpsmA |
| 643 | 2182 | | | mApsmUpsmGmCmCmAfAmAfAfCfAmAmC | 2222 | mApsfGpsmGmUmGfAmUmGmGmUmUmGmUfUmUfUmGmGm |
| | | | | mCmAmUmCmAmCmCmG-p-(ps)2-GalNAc4 | | CmAmUpsmCpsmA |
| 644 | 2183 | | | mGpsmApsmAmGmUmCfGmUfGfGfAmUmG | 2223 | vmUpsfApsmCmCmAfAmGmGmCmAmUmCmCfAmCfGmAmCmU |
| | | | | mCmCmUmUmGmGmUmA-p-(ps)2-GalNAc4 | | mUmCpsmGpsmU |
| 645 | 2184 | | | mGpsmApsmAmGmUmCfGmUfGfGfAmUmG | 2224 | vmApsfApsmCmCmAfAmGmGmCmAmUmCmCfAmCfGmAmCmU |
| | | | | mCmCmUmUmGmGmUmA-p-(ps)2-GalNAc4 | | mUmCpsmGpsmU |
| 646 | 2185 | | | mGpsmApsmAmGmUmCfGmUfGfGfAmUmG | 2225 | mApsfApsmCmCmAfAmGmGmCmAmUmCmCfAmCfGmAmCmUm |
| | | | | mCmCmUmUmGmGmUmA-p-(ps)2-GalNAc4 | | UmCpsmGpsmU |
| 647 | 2186 | | | mUpsmCpsmGmUmGmGfAmUfGfCfCmUmU | 2226 | mApsfApsmCmAmUfAmCmCmAmAmGmGmCfAmUfCmCmAmCm |
| | | | | mGmGmUmAmUmGmUmU-p-(ps)2- | | GmApsmCpsmU |
| | | | | GalNAc4 | | |
| 648 | 2187 | | | mApsmGpsmUmCmGmUfGmGfAfUfGmCmC | 2227 | mApsfApsmUmAmCfCmAmAmGmGmCmAmUfCmCfAmCmGmA |
| | | | | mUmUmGmGmUmAmUmG-p-(ps)2- | | mCmUpsmUpsmC |
| | | | | GalNAc4 | | |
| 649 | 2278 | | | mUpsmCpsmCmAmAmAfGmAfCfGfAmAmG | 2302 | mApsfUpsmCmCmAfCmGmAmCmUmUmCmGfUmCfUmUmUmG |
| | | | | mUmCmGmUmGmGmAmU-p-(ps)2- | | mGmApsmUpsmU |
| | | | | GalNAc4 | | |
| 650 | 2279 | | | mApsmUpsmGmCmCmAfAmAfAf2PfAmAmC | 2303 | vmApsfGpsmGmUmGfAmUmGmGmUmUmGmUfUmUfUmGmG |
| | | | | mCmAmUmCmAmCmCmG-p-(ps)2-GalNAc4 | | mCmAmUpsmCpsmA |
| 651 | 2280 | | | mApsmUpsmGmCmCmAfAmAfAfCfAmAmC | 2304 | vmApsfGpsmGmUmGfAmUmGmGmUmUmGmUfUmUfUmGmG |
| | | | | mCmAmUmCmAmun34CmCmG-p-(ps)2- | | mCmAmUpsmCpsmA |
| | | | | GalNAc4 | | |
| 652 | 2281 | | | mApsmUpsmGmCmCmAfAmAfAfCfAmAmC | 2305 | vmApsfGpsmGmUmGfAmUmGmGmUmUmGmUfUmUfUmGmG |
| | | | | mCmAmUmCmAmCmCmU-p-(ps)2-GalNAc4 | | mCmAmUpsmCpsmA |
| 653 | 2282 | | | mApsmUpsmGmCmCmAfAmAfAf2PfAmAmC | 2306 | vmApsfGpsmGmUmGfAmUmGmGmUmUmGmUfUmUfUmGmG |
| | | | | mCmAmUmCmAmCmCmU-p-(ps)2-GalNAc4 | | mCmAmUpsmCpsmA |
| 654 | 2283 | | | mGpsmApsmAmGmUmCfGmUfGfGfAmUmG | 2307 | c20- |
| | | | | mCmCmUmUmGmGmUmA-p-(ps)2-GalNAc4 | | 4hUpsfApsmCmCmAfAmGmGmCmAmUmCmCfAmCfGmAmCmU |
| | | | | | | mUmCpsmGpsmU |
| 655 | 2284 | | | mGpsmApsmAmGmUmCfGmUfGfGfAmUmG | 2308 | vmApsfApsmCmCmAfAunGmGmCmAmUmCmCfAmCfGmAmCmU |
| | | | | mCmCmUmUmGmGmUmA-p-(ps)2-GalNAc4 | | mUmCpsmGpsmU |
| 656 | 2285 | | | mGpsmApsmAmGmUmCfGmUfGfGfAmUmG | 2309 | vmApsfApsmCmCmAfAmGmGmCmAmUmCmCfAmCfGmAmCmU |
| | | | | mCmCmUmUmGmGmUmU-p-(ps)2- | | mUmCpsmGpsmU |
| | | | | GalNAc4 | | |
| 657 | 2286 | | | mGpsmApsmAmGmUmCfGmUfGfGfCmUmG | 2310 | vmApsfApsmCmCmAfAmGmGmCmAmUmCmCfAmCfGmAmCmU |
| | | | | mCmCmUmUmGmGmUmA-p-(ps)2-GalNAc4 | | mUmCpsmGpsmU |
| 658 | 2287 | | | mGpsmApsmAmGmUmCfGmUfGfGfAmUmG | 2311 | vmApsfApsmCmCmAfAmGmGmCmAmUmCmCfAmCfGmAmCmU |
| | | | | mCmCmUmUmGmun34GmUmA-p-(ps)2- | | mUmCpsmGpsmU |
| | | | | GalNAc4 | | |
| 659 | 2288 | | | mGpsmApsmAmGmUmCfGmUfGfGfAmUmG | 2312 | mApsfApsmCmCmAfAmGmGmCmAmUmCmCfAmCfGmAmCmUm |
| | | | | mCmCmUmUmGmGmUmU-p-(ps)2- | | UmCpsmGpsmU |
| | | | | GalNAc4 | | |
| 660 | 2289 | | | mGpsmApsmAmGmUmCfGmUfGfGfCmUmG | 2313 | mApsfApsmCmCmAfAmGmGmCmAmUmCmCfAmCfGmAmCmUm |
| | | | | mCmCmUmUmGmGmUmA-p-(ps)2-GalNAc4 | | UmCpsmGpsmU |
| 661 | 2290 | | | mGpsmApsmAmGmUmCfGmUfGfGfAmUmG | 2314 | mApsfApsmCmCmAfAmGmGmCmAmUmCmCfAmCfGmAmCmUm |
| | | | | mCmCmUmUmGmun34GmUmA-p-(ps)2- | | UmCpsmGpsmU |
| | | | | GalNAc4 | | |
| 662 | 2291 | | | mGpsmUpsmCmGmUmGfGmAfUfGfCmCmU | 2315 | vmApsfCpsmAmUmAfCmCmAmAmGmGmCmAfUmCfCmAmCmG |
| | | | | mUmGmGmUmAmUmGmU-p-(ps)2- | | mAmCpsmUpsmU |
| | | | | GalNAc4 | | |
| 663 | 2186 | | | mUpsmCpsmGmUmGmGfAmUfGfCfCmUmU | 2024 | mApsfApsmCmAmUfAmCmCmAmAmGmGmCfAmUfCmCmAmCm |
| | | | | mGmGmUmAmUmGmUmU-p-(ps)2- | | GmApsmUpsmU |
| | | | | GalNAc4 | | |
| 664 | 2292 | | | mUpsmCpsmGmUmGmGfAmUfGfCfCmUmU | 2316 | vmApsfApsmCmAmUfAmCmCmAmAmGmGmCfAmUfCmCmAmC |
| | | | | mGmGmUmAmUmGmUmU-p-(ps)2- | | mGmApsmUpsmU |
| | | | | GalNAc4 | | |
| 665 | 2293 | | | mApsmUpsmGmGfAmGfGfAfGmUmGmAm | 2317 | mUpsfUpsmGmUmCfAmCmUmCmAmCmUmCfCmUfCmCmAmUp |
| | | | | GmUmGmAmCmAmA-p-(ps)2-GalNAc4 | | smUpsmU |
| 666 | 2294 | | | mApsmUpsmGmCmCmAfAmAfAfCfAmAmC | 2318 | mCpsfGpsmGmUmGfAmUmGmGmUmUmGmUfUmUfUmGmGm |
| | | | | mCmAmUmCmAmCmCmG-p-(ps)2-GalNAc4 | | CmAmUpsmUpsmU |
| 667 | 2295 | | | mGpsmApsmAmGfUmCmGmUfGfGfAmUmG | 2319 | mUpsfApsmCmCmAmAmGmGmCmAmUmCmCfAmCmGmAmCm |
| | | | | fCmCmUmUmGfGmUmA-p-(ps)2-GalNAc4 | | UmUmCpsmUpsmU |
| 668 | 2296 | | | mGpsmApsmAmGfUmCmGmUfGfGfAmUmG | 2320 | vmUpsfApsmCmCmAmAmGmGmCmAmUmCmCfAmCmGmAmC |
| | | | | fCmCmUmUmGfGmUmA-p-(ps)2-GalINAc4 | | mUmUmCpsmUpsmU |
| 669 | 2297 | | | mGpsmUpsmGmUmCmUfGmAfCfUfUmUmC | 2321 | mUpsfUpsmUmGmGfAmCmCmGmAmAmAmGfUmCfAmGmAmC |
| | | | | mGmGmUmCmCmAmAmA-p-(ps)2-GalNAc4 | | mAmCpsmUpsmU |
| 670 | 2298 | | | mUpsmCpsmCmAmAmAfGmAfCfGfAmAmG | 2322 | vmApsfUpsmCmCmAfCmGmAmCmUmUmCmGfUmCfUmUmUmG |
| | | | | mUmCmGmUmGmGmAmU-p-(ps)2- | | mGmApsmUpsmU |
| | | | | GalNAc4 | | |
| 671 | 2299 | | | mGpsmUpsmCmGmUmGfGmAfUfGfCmCmU | 2323 | mApsfCpsmAmUmAfCmCmAmAmGmGmCmAfUmCfCmAmCmGm |
| | | | | mUmGmGmUmAmUmGmU-p-(ps)2- | | AmCpsmUpsmU |
| | | | | GalNAc4 | | |
| 672 | 2300 | | | mApsmUpsmGmCmCmAfAmAfAfCfAmAmC | 2324 | vmCpsfGpsmGmUmGfAmUmGmGmUmUmGmUfUmUfUmGmGm |
| | | | | mCmAmUmCmAmCmCmG-p-(ps)2-GalNAc4 | | CmAmUpsmUpsmU |
| 673 | 2301 | | | mGpsmApsmAmGmUmCfGmUfGfGfAmUmG | 2325 | vmUpsfApsmCmCmAfAmGmGmCmAmUmCmCfAmCfGmAmCmU |
| | | | | mCmCmUmUmGmGmUmA-p-(ps)2-GalNAc4 | | mUmCpsmUpsmU |
| 674 | 2326 | | | mApsmCpsmAmUfCmUfGfCfCmCmUmAmA | 2340 | mUpsfUpsmGmAmCfUmUmUmAmGmGmGmCfAmGfAmUmGmU |
| | | | | mAmGmUmCmAmA-p-(ps)2-GalNAc4 | | psmCpsmG |
| 675 | 2327 | | | mApsmCpsmAmUfCmUfGfCfCmCmUmAmA | 2341 | vmUpsfUpsmGmAmCfUmUmUmAmGmGmGmCfAmGfAmUmGm |
| | | | | mAmGmUmCmAmA-p-(ps)2-GalNAc4 | | UpsmCpsmG |
| 676 | 2328 | | | mApsmCpsmAmUfCmUfGfCfCmCmUmAmA | 2342 | mApsfUpsmGmAmCfUmUmUmAmGmGmGmCfAmGfAmUmGmU |
| | | | | mAmGmUmCmAmA-p-(ps)2-GalNAc4 | | psmCpsmG |
| 677 | 2329 | | | mApsmCpsmAmUfCmUfGfCfCmCmUmAmA | 2343 | vmApsfUpsmGmAmCfUmUmUmAmGmGmGmCfAmGfAmUmGm |
| | | | | mAmGmUmCmAmA-p-(ps)2-GalNAc4 | | UpsmCpsmG |
| 678 | 2330 | | | mApsmCpsmAmUf2PmUfGfCfCmCmUmAm | 2344 | mUpsfUpsmGmAmCfUmUmUmAmGmGmGmCfAmGfAmUmGmU |
| | | | | AmAmGmUmCmAmA-p-(ps)2-GalNAc4 | | psmCpsmG |
| 679 | 2331 | | | mApsmCpsmAmUf2PmUfGfCfCmCmUmAm | 2345 | vmUpsfUpsmGmAmCfUmUmUmAmGmGmGmCfAmGfAmUmGm |
| | | | | AmAmGmUmCmAmA-p-(ps)2-GalNAc4 | | UpsmCpsmG |
| 680 | 2332 | | | mApsmCpsmAmUfCmUfGfCf2PmCmUmAm | 2346 | mUpsfUpsmGmAmCfUmUmUmAmGmGmGmCfAmGfAmUmGmU |
| | | | | AmAmGmUmCmAmA-p-(ps)2-GalNAc4 | | psmCpsmG |
| 681 | 2333 | | | mApsmCpsmAmUfCmUfGfCf2PmCmUmAm | 2347 | vmUpsfUpsmGmAmCfUmUmUmAmGmGmGmCfAmGfAmUmGm |
| | | | | AmAmGmUmCmAmA-p-(ps)2-GalNAc4 | | UpsmCpsmG |
| 682 | 2334 | | | mGpsmCpsmCmUfGmUfGfGfAmAmUmCmU | 2348 | mApsfApsmUmGmGfCmAmGmAmUmUmCmCfAmCfAmGmGmCp |
| | | | | mGmCmCmAmUmU-p-(ps)2-GalNAc4 | | smApsmG |
| 683 | 2335 | | | mGpsmCpsmCmUfGmUfGfGfAmAmUmCmU | 2349 | vmApsfApsmUmGmGfCmAmGmAmUmUmCmCfAmCfAmGmGmC |
| | | | | mGmCmCmAmUmU-p-(ps)2-GalNAc4 | | psmApsmG |
| 684 | 2336 | | | mGpsmCpsmCmUfGmUfGfGfAmAmUmCmU | 2350 | mUpsfApsmUmGmGfCmAmGmAmUmUmCmCfAmCfAmGmGmC |
| | | | | mGmCmCmAmUmU-p-(ps)2-GalNAc4 | | psmApsmG |
| 685 | 2337 | | | mGpsmCpsmCmUfGmUfGfGfAmAmUmCmU | 2351 | vmUpsfApsmUmGmGfCmAmGmAmUmUmCmCfAmCfAmGmGmC |
| | | | | mGmCmCmAmUmU-p-(ps)2-GalNAc4 | | psmApsmG |
| 686 | 2338 | | | mUpsmApsmCmCmAmGfAmGfUfGfUmCmU | 2352 | vmUpsfCpsmCmCmCfAmUmCmAmGmAmCmAfCmUfCmUmGmG |
| | | | | mGmAmUmGmGmGmGmA-p-(ps)2- | | mUmApsmApsmG |
| | | | | GalNAc4 | | |
| 687 | 2339 | | | mCpsmGpsmAmCmAmUfCmUfGfCfCmCmU | 2353 | vmUpsfUpsmGmAmCfUmUmUmAmGmGmGmCfAmGfAmUmGm |
| | | | | mAmAmAmGmUmCmAmA-p-(ps)2-GalNAc4 | | UmCmGpsmUpsmA |

### Example 60: In Vitro Assay of siRNA Activity

This example provides exemplary methods for determining the in vitro activity and possible cytotoxic effects of a subset of the siRNAs listed in Table 2. For example, the in vitro activity of the siRNAs may be determined by a luciferase reporter assay and/or a differential gene expression assay, which are described in greater detail below. Specifically, for example, the efficacy of each of the tested siRNA molecules in reducing (or downregulating) the expression of PNPLA3 in vitro was accessed. Each siRNA molecule tested consisted of a 19-mer or 21-mer duplex of two siRNA strands, the sense strand and the antisense strand, corresponding to certain siRNA Duplex ID Nos. in Table 2 above.

### Luciferase Reporter Assay in COS-7 Cells

Cell Culture, Plasmid Transfection, and siRNA Treatment

In the psiCHECK™-2 reporter plasmid, Renilla luciferase is used as the primary reporter gene with the PNPLA3 rs738409[G] gene (NM_025225.3:c.444C>G) (SEQ ID NO: 2067) cloned downstream of its translational stop codon. A second reporter gene, firefly luciferase, is also expressed and used as a transfection control.

COS-7 cells (ATCC, CRL-1651) were routinely cultured in Dulbecco's Modified Eagle's Medium (DMEM; Corning, 10-013-CM) supplemented with 10% fetal bovine serum (FBS; Gibco, 16000-044) and 1% Penicillin-Streptomycin (P/S; Corning, 30-002-CI) at 37° C. and 5% CO₂ until 80-90% confluency. Cells were then detached with 0.05% trypsin (Corning, 25-052-CV), resuspended in fresh DMEM, and seeded into 96-well microplates. Cells were transfected using Lipofectamine 3000 (Invitrogen, L30000001) with the psiCHECK™-2 reporter plasmid (Promega, C8021). The cells were then transfected with either 50 nM, 5 nM, or 0.5 nM of a siRNA duplex molecule using Lipofectamine RNAiMAX (Invitrogen, 13778100). A mock transfection control, which consisted of transfecting 1×phosphate-buffered saline, was included.

### Luciferase Reporter Activity

After about 72 hours of siRNA treatment, the Dual-Glo® Luciferase Assay System (Promega, E2940) was used according to the manufacturer's protocol to quantify firefly and Renilla luciferase activity. All luminescence was measured on an EnVision plate reader (Perkin Elmer). The Renilla:firefly luminescence ratio is calculated for each well. The ratios of siPNPLA3 wells are then normalized to ratios of the mock wells and percent inhibition was calculated.

Additionally, CellTiter-Glo© Luminescent Cell Viability Assays were also performed with similarly treated COS-7 cells to assess cytotoxic effects. Assays were performed according to the manufacturer's protocol and luminescence was measured on an EnVision plate reader. The luminescence from siRNA-treated wells were then normalized to luminescence of mock wells and percentage viability was calculated.

The results of the luciferase reporter assay and CellTiter-Glo viability assay in COS-7 cells are provided in Table 3 below.

TABLE 3
Luciferase Reporter Assay and CellTiter-
Glo Viability Assay in COS-7 Cells

| | Luciferase reporter assay | | | | | |
|---|---|---|---|---|---|---|
| siRNA ID | in COS-7 % inhibition | | | CellTiter-Glo in COS-7 | | |
| No. | of reporter activity | | | % viability | | |
| (MDx) | 50 nM | 5 nM | 0.5 nM | 50 nM | 5 nM | 0.5 nM |
| 1 | 0.43 | 12.52 | 8.02 | 97.03 | 101.10 | 98.22 |
| 2 | 7.63 | 5.29 | 12.29 | 90.34 | 99.17 | 96.78 |
| 3 | 17.21 | 9.25 | 0.86 | 98.03 | 96.75 | 102.97 |
| 4 | 3.40 | 10.47 | 1.69 | 104.87 | 96.99 | 104.42 |
| 5 | 9.28 | 8.17 | −2.77 | 98.61 | 99.76 | 96.98 |
| 6 | 14.38 | 4.52 | 2.75 | 103.59 | 102.78 | 105.46 |
| 7 | 7.67 | −4.43 | −7.86 | 101.67 | 99.44 | 101.49 |
| 8 | 5.23 | −10.87 | −9.26 | 99.59 | 96.29 | 103.56 |
| 9 | 22.29 | 14.11 | 8.85 | 102.46 | 107.79 | 109.56 |
| 10 | 0.86 | 6.60 | 9.27 | 108.25 | 113.71 | 102.61 |
| 11 | −5.83 | −2.62 | 7.43 | 101.33 | 103.80 | 102.97 |
| 12 | 21.98 | 12.86 | −13.63 | 106.31 | 105.53 | 101.76 |
| 13 | 29.53 | 29.93 | −4.18 | 103.58 | 104.17 | 109.45 |
| 14 | 53.43 | 49.35 | 4.46 | 94.87 | 103.77 | 102.00 |
| 15 | 29.27 | 23.53 | −0.77 | 107.28 | 106.11 | 103.86 |
| 16 | 29.58 | 27.61 | −2.23 | 104.39 | 106.40 | 103.88 |
| 17 | 5.28 | 8.68 | −21.74 | 105.58 | 100.80 | 105.39 |
| 18 | 42.35 | 34.03 | 8.24 | 115.85 | 109.57 | 104.21 |
| 19 | −10.08 | −13.46 | −3.66 | 99.23 | 101.11 | 94.47 |
| 20 | 18.43 | 18.99 | −3.85 | 90.25 | 102.20 | 102.98 |
| 21 | −15.56 | −25.66 | −29.75 | 97.58 | 101.05 | 94.21 |
| 22 | 19.79 | 25.20 | −0.17 | 100.31 | 100.83 | 96.00 |
| 23 | 21.67 | 5.38 | −3.78 | 99.90 | 104.61 | 96.50 |
| 24 | 5.71 | 14.67 | −2.70 | 105.02 | 102.73 | 109.65 |
| 25 | −12.98 | −8.70 | −5.60 | 103.00 | 100.81 | 104.16 |
| 26 | −18.55 | −10.07 | −14.57 | 105.05 | 98.13 | 109.31 |
| 27 | −12.18 | 4.53 | −5.38 | 100.92 | 102.88 | 108.52 |
| 28 | −12.59 | 12.10 | 5.70 | 98.87 | 99.39 | 97.51 |
| 29 | 6.58 | 1.86 | 9.72 | 105.01 | 102.36 | 98.33 |
| 30 | 5.17 | 3.35 | −7.51 | 104.85 | 102.71 | 99.25 |
| 31 | −0.66 | 3.24 | −6.74 | 100.04 | 102.95 | 99.07 |
| 32 | −7.11 | −7.52 | −7.59 | 101.29 | 100.03 | 101.68 |
| 33 | 14.17 | 17.68 | 8.53 | 100.36 | 103.04 | 98.21 |
| 34 | 2.81 | 6.64 | 9.12 | 97.60 | 100.21 | 99.83 |
| 35 | 5.17 | 9.17 | −1.13 | 97.89 | 101.16 | 97.09 |
| 36 | 12.72 | 18.81 | 18.14 | 99.49 | 100.62 | 103.86 |
| 37 | 6.34 | −22.97 | −4.01 | 93.51 | 96.88 | 95.46 |
| 38 | 4.96 | −40.97 | −13.92 | 96.14 | 92.72 | 93.71 |
| 39 | −7.20 | −9.11 | −25.14 | 96.68 | 96.49 | 96.59 |
| 40 | −5.30 | 13.33 | −4.20 | 96.42 | 95.66 | 98.94 |
| 41 | 6.32 | −3.27 | −11.22 | 96.98 | 94.81 | 97.72 |
| 42 | −2.73 | 2.34 | −6.01 | 99.34 | 98.05 | 96.56 |
| 43 | 4.64 | 10.30 | −7.59 | 94.41 | 94.17 | 96.36 |
| 44 | 3.22 | 10.31 | −0.07 | 98.21 | 95.43 | 99.38 |
| 45 | −10.11 | 10.11 | −2.65 | 100.53 | 101.25 | 100.78 |
| 46 | −3.32 | 6.18 | −15.76 | 96.01 | 94.42 | 100.54 |
| 47 | 9.37 | 10.77 | −3.48 | 94.89 | 97.14 | 94.13 |
| 48 | 2.57 | −8.48 | −20.16 | 96.19 | 96.86 | 101.26 |
| 49 | 0.17 | 3.68 | −4.29 | 97.82 | 98.20 | 95.54 |
| 50 | −0.54 | 12.19 | −4.37 | 100.57 | 94.02 | 95.14 |
| 51 | 17.18 | 16.45 | 3.29 | 96.25 | 96.30 | 98.27 |
| 52 | −2.81 | −2.17 | −3.46 | 99.64 | 94.26 | 96.67 |
| 53 | −1.20 | 1.40 | −2.86 | 104.44 | 94.65 | 91.65 |
| 54 | 14.92 | 7.10 | −1.56 | 101.99 | 98.46 | 99.24 |
| 55 | 6.97 | 11.45 | 3.34 | 98.27 | 102.09 | 100.38 |
| 56 | −9.50 | 12.33 | 2.51 | 100.08 | 97.78 | 99.31 |
| 57 | 7.68 | −0.32 | −2.53 | 95.06 | 101.76 | 100.68 |
| 58 | −2.50 | −14.43 | −4.44 | 103.71 | 105.13 | 101.11 |
| 59 | 5.07 | −5.88 | −8.51 | 101.95 | 104.15 | 100.15 |
| 60 | −0.68 | 0.45 | −1.50 | 100.53 | 106.99 | 101.34 |
| 61 | −2.62 | 5.58 | −3.80 | 100.10 | 102.51 | 97.71 |
| 62 | 10.73 | 3.60 | 1.45 | 98.91 | 100.72 | 99.30 |
| 63 | 36.37 | 31.94 | 3.55 | 96.30 | 103.51 | 98.75 |
| 64 | 19.78 | 18.08 | 4.72 | 91.19 | 95.25 | 94.81 |
| 65 | −3.62 | 13.54 | 4.24 | 96.16 | 93.63 | 94.95 |
| 66 | 40.71 | 30.72 | 0.95 | 96.22 | 96.60 | 93.00 |
| 67 | 11.52 | 2.03 | −10.57 | 96.24 | 98.16 | 92.48 |
| 68 | 41.47 | 30.22 | 3.94 | 96.42 | 98.12 | 97.32 |
| 69 | 22.57 | 18.99 | −5.26 | 96.77 | 97.19 | 95.04 |
| 70 | 45.39 | 26.33 | 0.46 | 97.87 | 98.27 | 95.04 |
| 71 | 31.86 | 35.78 | −0.17 | 98.25 | 96.56 | 94.46 |
| 72 | −0.52 | −3.48 | −17.64 | 96.33 | 99.39 | 100.97 |
| 73 | 10.93 | 12.96 | 17.35 | 103.58 | 122.28 | 101.90 |
| 74 | 38.48 | 32.25 | 11.66 | 105.50 | 113.90 | 97.40 |
| 75 | 37.72 | 26.60 | 6.29 | 107.30 | 117.53 | 94.58 |
| 76 | 25.94 | 15.17 | 10.79 | 103.87 | 110.01 | 101.92 |
| 77 | 37.18 | 23.01 | 17.27 | 101.73 | 103.78 | 112.03 |
| 78 | 22.02 | 23.44 | −0.22 | 90.92 | 109.41 | 92.02 |
| 79 | 35.58 | 26.26 | 24.10 | 83.15 | 106.84 | 92.82 |
| 80 | 23.15 | 12.46 | −0.12 | 86.89 | 111.44 | 87.72 |
| 81 | 41.54 | 19.27 | −11.96 | 89.40 | 100.08 | 92.58 |
| 82 | 30.54 | 11.55 | 2.43 | 129.50 | 125.11 | 116.66 |
| 83 | 33.76 | 11.55 | −16.68 | 115.34 | 129.88 | 102.39 |
| 84 | 25.24 | 21.18 | −18.32 | 116.90 | 116.04 | 98.56 |
| 85 | 27.36 | 29.39 | −26.51 | 101.77 | 117.36 | 100.53 |
| 86 | 25.69 | 14.35 | −29.99 | 87.95 | 110.86 | 131.35 |
| 87 | 18.64 | 16.38 | −26.78 | 93.46 | 86.54 | 128.67 |
| 88 | 43.20 | 20.67 | −0.67 | 77.03 | 92.96 | 117.07 |
| 89 | 17.07 | 16.81 | −9.67 | 100.22 | 108.68 | 112.51 |
| 90 | 40.55 | 26.27 | −7.31 | 77.42 | 99.33 | 112.08 |
| 91 | 40.04 | 45.54 | 23.02 | 103.60 | 111.24 | 119.24 |
| 92 | 31.06 | 40.38 | 22.75 | 108.91 | 112.75 | 113.75 |
| 93 | 45.34 | 51.19 | 24.30 | 100.50 | 105.73 | 104.57 |
| 94 | 41.69 | 28.65 | 20.82 | 97.95 | 81.20 | 99.36 |
| 95 | 50.36 | 50.47 | 18.89 | 105.24 | 83.74 | 86.54 |
| 96 | 57.17 | 42.37 | 20.05 | 91.95 | 76.88 | 79.52 |
| 97 | 40.26 | 28.72 | 4.57 | 87.77 | 86.96 | 93.42 |
| 98 | 57.27 | 46.03 | 22.54 | 84.63 | 90.32 | 97.17 |
| 99 | 51.27 | 43.51 | 17.90 | 90.61 | 98.14 | 107.90 |
| 100 | 45.21 | 47.67 | 21.58 | 111.01 | 108.36 | 109.82 |
| 101 | 37.93 | 30.64 | 26.92 | 109.70 | 93.51 | 110.01 |
| 102 | 50.27 | 46.47 | 31.49 | 117.58 | 101.35 | 116.44 |
| 103 | 40.84 | 37.28 | 13.81 | 97.71 | 107.57 | 112.60 |
| 104 | 60.15 | 47.21 | 18.00 | 113.24 | 101.53 | 119.28 |
| 105 | 57.09 | 40.55 | 26.61 | 110.20 | 111.53 | 118.37 |
| 106 | 66.20 | 55.15 | 27.53 | 106.54 | 98.13 | 118.24 |
| 107 | 65.89 | 51.41 | 32.81 | 110.86 | 116.91 | 108.67 |
| 108 | 51.36 | 52.20 | 11.19 | 106.74 | 99.18 | 110.05 |
| 109 | 60.40 | 54.44 | 24.94 | 103.70 | — | — |
| 110 | 38.23 | 28.87 | 0.75 | 103.14 | — | — |
| 111 | 15.24 | 12.25 | −1.66 | 107.56 | — | — |
| 112 | 60.94 | 59.26 | 23.39 | 104.59 | — | — |
| 113 | 39.34 | 37.62 | 9.30 | 108.62 | — | — |
| 114 | 30.16 | 31.55 | 3.44 | 105.98 | — | — |
| 115 | 49.85 | 42.32 | 12.79 | 100.04 | — | — |
| 116 | 49.72 | 50.73 | −3.51 | 93.84 | — | — |
| 117 | 35.57 | 27.40 | 6.94 | 104.45 | — | — |
| 118 | 44.04 | 43.02 | 20.85 | 102.27 | — | — |
| 119 | 55.26 | 56.81 | 16.27 | 90.32 | — | — |
| 120 | 20.98 | 23.16 | −0.21 | 98.91 | — | — |
| 121 | 68.22 | 67.49 | 42.00 | 97.32 | — | — |
| 122 | 50.00 | 41.37 | 6.89 | 102.44 | — | — |
| 123 | 39.23 | 38.14 | 5.10 | 103.50 | — | — |
| 124 | 73.58 | 69.20 | 23.53 | 101.17 | — | — |
| 125 | 44.45 | 35.69 | −8.51 | 93.53 | — | — |
| 126 | 31.09 | 30.88 | −7.55 | 101.28 | — | — |
| 127 | 10.93 | 11.39 | 10.79 | 100.25 | — | — |
| 128 | 25.17 | 32.19 | 14.86 | 99.77 | — | — |
| 129 | 29.33 | 22.99 | 0.61 | 100.75 | — | — |
| 130 | −0.73 | 19.30 | 11.71 | 95.68 | — | — |
| 131 | 20.11 | 14.50 | 1.67 | 100.61 | — | — |
| 132 | 22.95 | 10.15 | 5.53 | 102.59 | — | — |
| 133 | 37.57 | 20.43 | 2.55 | 103.12 | — | — |
| 134 | 16.29 | 2.33 | −12.67 | 100.21 | — | — |
| 135 | 35.47 | 20.60 | 6.02 | 99.74 | — | — |
| 136 | 25.77 | 17.38 | 4.47 | 100.91 | — | — |
| 137 | 20.45 | 13.54 | −6.69 | 106.87 | — | — |
| 138 | 40.96 | 25.39 | 1.99 | 104.23 | — | — |
| 139 | 22.31 | 8.31 | −0.69 | 98.82 | — | — |
| 140 | 8.15 | 10.62 | −3.53 | 106.20 | — | — |
| 141 | 58.95 | 56.34 | 20.90 | 102.54 | — | — |
| 142 | 59.19 | 58.25 | 11.58 | 112.00 | — | — |
| 143 | 61.64 | 53.55 | 3.43 | 108.92 | — | — |
| 144 | 42.55 | 29.39 | 4.42 | 104.60 | — | — |
| 145 | 28.74 | 24.39 | 13.22 | 90.32 | 96.97 | 96.45 |
| 146 | 39.27 | 40.83 | 14.56 | 91.13 | 100.64 | 102.93 |
| 147 | 43.55 | 47.77 | 28.66 | 98.77 | 103.52 | 100.95 |
| 148 | 12.45 | 20.01 | 9.30 | 97.25 | 102.76 | 102.06 |
| 149 | 53.38 | 25.42 | −0.13 | 97.28 | 102.59 | 104.59 |
| 150 | 53.51 | 57.75 | 26.50 | 96.49 | 103.51 | 103.85 |
| 151 | 9.94 | 9.01 | 6.02 | 95.50 | 103.24 | 103.18 |
| 152 | −9.62 | −1.24 | −1.37 | 98.49 | 104.17 | 103.38 |
| 153 | 9.77 | 16.33 | 3.08 | 96.34 | 101.30 | 105.22 |
| 154 | −5.26 | −3.21 | 5.74 | 99.77 | 104.06 | 97.77 |
| 155 | −7.72 | 3.55 | 7.01 | 94.44 | 98.33 | 97.78 |
| 156 | −13.36 | −10.16 | −3.65 | 97.83 | 97.94 | 100.17 |
| 157 | 21.66 | 16.08 | 10.06 | 97.51 | 99.76 | 105.20 |
| 158 | −2.64 | 1.40 | −4.71 | 101.01 | 100.76 | 104.79 |
| 159 | 7.65 | 5.54 | −6.29 | 101.47 | 103.96 | 102.77 |
| 160 | 3.74 | 1.31 | 3.04 | 104.10 | 103.93 | 103.82 |
| 161 | 5.27 | −7.43 | 3.75 | 93.68 | 105.41 | 105.87 |
| 162 | −4.85 | 14.55 | 12.57 | 96.56 | 102.35 | 107.58 |
| 163 | 3.64 | −1.01 | 6.69 | 96.47 | 105.02 | 96.58 |
| 164 | 19.94 | 14.29 | −20.90 | 97.26 | 103.66 | 98.77 |
| 165 | −2.94 | 8.01 | −6.85 | 100.48 | 100.25 | 103.26 |
| 166 | 13.07 | 8.27 | 8.56 | 97.24 | 101.95 | 107.44 |
| 167 | −11.94 | −10.11 | −1.99 | 98.73 | 108.28 | 109.80 |
| 168 | 40.04 | 33.48 | 21.62 | 99.91 | 101.93 | 109.26 |
| 169 | 2.60 | 10.99 | 18.78 | 101.59 | 100.08 | 106.07 |
| 170 | 31.14 | 38.50 | 11.08 | 99.03 | 100.77 | 106.39 |
| 171 | 48.93 | 62.09 | 40.81 | 98.12 | 103.37 | 108.89 |
| 172 | 35.17 | 33.18 | 18.62 | 96.25 | 98.15 | 96.41 |
| 173 | 34.85 | 34.54 | 7.11 | 96.99 | 98.30 | 98.40 |
| 174 | 28.15 | −1.56 | −3.64 | 109.52 | 102.35 | 100.49 |
| 175 | −2.78 | 15.47 | 1.99 | 103.73 | 102.04 | 101.67 |
| 176 | −10.70 | −11.26 | −5.19 | 99.39 | 102.57 | 102.65 |
| 177 | −21.12 | 8.29 | 7.94 | 101.53 | 98.37 | 104.29 |
| 178 | −4.96 | 6.95 | 9.32 | 99.59 | 99.03 | 102.11 |
| 179 | −11.56 | 14.95 | 13.32 | 97.18 | 98.74 | 104.08 |
| 180 | −22.55 | −2.19 | 3.86 | 99.68 | 102.83 | 104.42 |
| 181 | 18.33 | 23.72 | 11.96 | 98.45 | 95.23 | 95.61 |
| 182 | 7.21 | 12.94 | 1.33 | 98.76 | 96.15 | 93.81 |
| 183 | −1.73 | 10.67 | −1.39 | 92.49 | 98.16 | 91.24 |
| 184 | 24.72 | 36.82 | 7.02 | 93.66 | 97.05 | 94.16 |
| 185 | 7.63 | 22.45 | −1.97 | 101.95 | 104.06 | 99.53 |
| 186 | 14.07 | 14.14 | 2.39 | 101.64 | 104.84 | 101.03 |
| 187 | 4.71 | 14.29 | 1.10 | 96.57 | 98.02 | 92.58 |
| 188 | 39.36 | 31.71 | 1.00 | 98.72 | 97.00 | 90.91 |
| 189 | 55.67 | 50.23 | 27.97 | 98.54 | 102.30 | 94.68 |
| 190 | 15.10 | 9.35 | 3.37 | 93.58 | 101.74 | 95.54 |
| 191 | 41.06 | 36.70 | 6.72 | 99.69 | 104.22 | 89.84 |
| 192 | 41.76 | 38.34 | 11.22 | 96.93 | 99.47 | 90.58 |
| 193 | 44.50 | 38.17 | −1.24 | 96.90 | 97.12 | 93.72 |
| 194 | 12.35 | 14.97 | 10.19 | 104.04 | 104.22 | 96.76 |
| 195 | 38.24 | 28.80 | −0.41 | 105.23 | 107.31 | 102.01 |
| 196 | 11.11 | 10.08 | −7.50 | 99.60 | 98.91 | 93.16 |
| 197 | 13.09 | 14.58 | −11.09 | 101.05 | 101.08 | 94.76 |
| 198 | 23.77 | 26.43 | 1.81 | 102.54 | 105.85 | 98.23 |
| 199 | 38.07 | 46.54 | 2.95 | 97.45 | 99.16 | 92.95 |
| 200 | 2.08 | 11.30 | −13.24 | 91.58 | 95.91 | 90.60 |
| 201 | −10.78 | 7.52 | −26.00 | 93.28 | 94.54 | 93.11 |
| 202 | −7.76 | 1.29 | −17.80 | 98.07 | 94.29 | 93.99 |
| 203 | 8.48 | 13.54 | −22.46 | 99.13 | 98.11 | 100.67 |
| 204 | 16.17 | 16.71 | −11.84 | 101.64 | 103.54 | 102.29 |
| 205 | 1.49 | 20.89 | −33.42 | 97.38 | 98.98 | 92.53 |
| 206 | 38.54 | 29.63 | −29.97 | 97.23 | 97.34 | 99.26 |
| 207 | −1.37 | 3.03 | −16.26 | 98.84 | 107.05 | 101.12 |
| 208 | 45.85 | 45.15 | 5.10 | 91.07 | 101.35 | 91.20 |
| 209 | 9.61 | 10.69 | −23.36 | 101.05 | 104.94 | 98.16 |
| 210 | 21.93 | 8.97 | −14.33 | 104.85 | 102.56 | 98.34 |
| 211 | 13.50 | 6.63 | −9.80 | 103.74 | 98.85 | 96.40 |
| 212 | 29.26 | 35.40 | −4.10 | 103.38 | 103.19 | 106.45 |
| 213 | 20.77 | 8.45 | −14.89 | 103.91 | 107.83 | 106.27 |
| 214 | 23.40 | 16.59 | −20.18 | 105.23 | 102.33 | 97.17 |
| 215 | 1.29 | 11.46 | −26.44 | 102.20 | 102.14 | 94.33 |
| 216 | 1.33 | 13.43 | −10.08 | 99.43 | 98.72 | 105.63 |
| 217 | −2.30 | −0.33 | 7.10 | 101.59 | 104.17 | 90.33 |
| 218 | 50.42 | 54.87 | 22.10 | 98.70 | 98.34 | 93.18 |
| 219 | 24.51 | 32.49 | −0.87 | 101.62 | 101.26 | 94.87 |
| 220 | 44.23 | 43.30 | 14.24 | 100.96 | 101.52 | 97.46 |
| 221 | 61.13 | 58.20 | 23.60 | 102.00 | 101.71 | 107.93 |
| 222 | 84.81 | 78.32 | 45.07 | 104.16 | 104.25 | 106.29 |
| 223 | 72.48 | 70.75 | 41.27 | 101.42 | 105.09 | 104.46 |
| 224 | 7.33 | 0.84 | −7.20 | 99.01 | 101.96 | 105.23 |
| 225 | 11.91 | 13.73 | 8.63 | 101.00 | 103.72 | 103.58 |
| 226 | 16.19 | 20.67 | −4.17 | 96.79 | 97.92 | 93.05 |
| 227 | 51.81 | 54.04 | 27.20 | 95.34 | 93.93 | 100.37 |
| 228 | −12.80 | −0.92 | −7.23 | 101.01 | 94.02 | 91.67 |
| 229 | −13.24 | 8.90 | 3.86 | 95.92 | 101.58 | 87.51 |
| 230 | −41.29 | −1.20 | −8.77 | 98.01 | 103.73 | 96.38 |
| 231 | 22.12 | 14.46 | 6.15 | 99.59 | 95.05 | 97.41 |
| 232 | 21.97 | 8.22 | −11.18 | 99.07 | 97.81 | 92.20 |
| 233 | 23.04 | −6.66 | −27.62 | 95.31 | 91.27 | 97.85 |
| 234 | 21.33 | 0.14 | −22.08 | 95.80 | 98.01 | 90.20 |
| 235 | 19.80 | −6.64 | −32.32 | 94.09 | 94.67 | 93.95 |
| 236 | 14.94 | −4.37 | −29.08 | 97.41 | 105.07 | 99.35 |
| 237 | 20.89 | −0.35 | −22.71 | 101.40 | 104.76 | 100.06 |
| 238 | 18.99 | 3.26 | −18.47 | 98.23 | 104.93 | 97.68 |
| 239 | 6.04 | −0.56 | −9.81 | 98.02 | 106.08 | 101.99 |
| 240 | 38.64 | 39.23 | 21.66 | 87.98 | 97.92 | 89.46 |
| 241 | 31.51 | 25.43 | 19.85 | 96.82 | 94.90 | 96.68 |
| 242 | 45.17 | 46.14 | 14.54 | 93.01 | 96.67 | 91.63 |
| 243 | 33.07 | 29.08 | −6.58 | 99.59 | 101.03 | 91.56 |
| 244 | 33.84 | 17.88 | −0.52 | 101.84 | 100.63 | 89.78 |
| 245 | 37.16 | 30.12 | −11.43 | 104.41 | 102.31 | 105.14 |
| 246 | 27.25 | 34.93 | 5.33 | 102.08 | 104.30 | 104.76 |
| 247 | 23.44 | 14.46 | −10.51 | 98.85 | 101.44 | 97.86 |
| 248 | 13.31 | 14.44 | −19.94 | 98.21 | 103.56 | 103.54 |
| 249 | 14.13 | 2.98 | −7.14 | 90.47 | 102.69 | 90.93 |
| 250 | 0.00 | 5.76 | −8.15 | 97.07 | 106.82 | 95.37 |
| 251 | 13.63 | 15.78 | −2.17 | 96.23 | 106.20 | 97.45 |
| 252 | 27.07 | 22.13 | 12.67 | 98.27 | 107.51 | 102.54 |
| 253 | 67.21 | 66.81 | 38.53 | 103.51 | 107.05 | 103.60 |
| 254 | 43.54 | 42.90 | 22.73 | 105.76 | 106.68 | 103.90 |
| 255 | 27.52 | 28.06 | −7.63 | 105.43 | 106.54 | 100.98 |
| 256 | 50.38 | 40.15 | −4.44 | 103.12 | 107.29 | 98.06 |
| 257 | 27.23 | 11.46 | 1.87 | 103.70 | 102.63 | 107.04 |
| 258 | 9.16 | 13.01 | 21.18 | 93.51 | 94.01 | 98.09 |
| 259 | 9.30 | 1.02 | 22.31 | 99.97 | 105.63 | 99.76 |
| 260 | 4.35 | −0.46 | 12.51 | 99.46 | 106.84 | 101.20 |
| 261 | 3.34 | 15.21 | 2.64 | 102.54 | 103.11 | 101.22 |
| 262 | 18.03 | 15.06 | 9.33 | 102.17 | 106.35 | 101.15 |
| 263 | 11.82 | 12.00 | −0.65 | 106.11 | 104.26 | 105.13 |
| 264 | 13.24 | 14.10 | 11.37 | 104.64 | 108.45 | 106.10 |
| 265 | −5.55 | 2.32 | 5.30 | 107.23 | 113.87 | 106.08 |
| 266 | 10.61 | 15.97 | −1.94 | 104.75 | 103.01 | 107.55 |
| 267 | −26.92 | −9.82 | −10.69 | 92.41 | 98.02 | 94.97 |
| 268 | −14.00 | −21.01 | −13.29 | 96.73 | 96.02 | 98.22 |
| 269 | −5.71 | −3.23 | −6.66 | 99.54 | 99.25 | 98.40 |
| 270 | 2.22 | 4.66 | −10.30 | 101.70 | 97.38 | 97.87 |
| 271 | −13.04 | −6.06 | −22.98 | 100.72 | 103.32 | 102.37 |
| 272 | −4.15 | −2.52 | −18.98 | 101.29 | 99.82 | 98.13 |
| 273 | −10.79 | −10.86 | −13.65 | 102.99 | 100.29 | 104.73 |
| 274 | −11.87 | −17.06 | 2.15 | 101.48 | 101.87 | 99.36 |
| 275 | 0.74 | 16.39 | −5.65 | 98.76 | 97.54 | 103.05 |
| 276 | −2.23 | 14.85 | 13.24 | 89.95 | 92.81 | 92.65 |
| 277 | −11.99 | −0.50 | 1.68 | 90.32 | 88.48 | 90.22 |
| 278 | 5.53 | 4.10 | 2.51 | 92.56 | 96.29 | 94.60 |
| 279 | 15.46 | 3.26 | 13.79 | 88.41 | 102.40 | 101.82 |
| 280 | 24.62 | 13.89 | 10.97 | 92.93 | 101.65 | 100.21 |
| 281 | 14.14 | 10.44 | 10.09 | 93.03 | 102.49 | 101.75 |
| 282 | 7.84 | 6.05 | 4.54 | 96.51 | 106.50 | 111.31 |
| 283 | 13.92 | 12.95 | 3.83 | 93.18 | 102.15 | 108.77 |
| 284 | 30.74 | 34.19 | 9.27 | 98.21 | 106.78 | 105.84 |
| 285 | −0.08 | 5.01 | 6.37 | 105.30 | 102.29 | 99.12 |
| 286 | 19.50 | 9.25 | 7.93 | 92.21 | 100.17 | 95.76 |
| 287 | −4.89 | −10.96 | −7.45 | 94.57 | 95.62 | 99.97 |
| 288 | 15.20 | 16.55 | 4.19 | 96.81 | 102.21 | 100.86 |
| 289 | 22.48 | 14.56 | 13.36 | 100.28 | 113.57 | 109.81 |
| 290 | −1.97 | 14.86 | −6.53 | 104.60 | 103.72 | 103.26 |
| 291 | 7.19 | 21.99 | 12.71 | 101.13 | 111.79 | 110.78 |
| 292 | 6.78 | 17.45 | 6.06 | 100.57 | 97.86 | 105.30 |
| 293 | 25.06 | 26.85 | 17.92 | 99.35 | 110.38 | 103.27 |
| 294 | 10.90 | 23.76 | 7.92 | 106.46 | 111.69 | 104.18 |
| 295 | 35.65 | 40.67 | 23.69 | 96.43 | 97.92 | 94.88 |
| 296 | 18.01 | 24.60 | 12.48 | 93.77 | 102.18 | 101.42 |
| 297 | −4.28 | −12.95 | 3.63 | 97.32 | 100.12 | 107.87 |
| 298 | 13.32 | 6.78 | 0.81 | 99.52 | 108.65 | 107.14 |
| 299 | −12.28 | −2.34 | −6.20 | 101.61 | 112.12 | 104.69 |
| 300 | 42.97 | 31.31 | −4.82 | 101.71 | 109.66 | 106.59 |
| 301 | −21.03 | −14.45 | −2.78 | 99.64 | 108.50 | 115.97 |
| 302 | −10.13 | −3.15 | 0.00 | 99.44 | 110.97 | 111.18 |
| 303 | 13.59 | 16.07 | 1.26 | 98.83 | 104.71 | 107.08 |
| 304 | 2.38 | 9.03 | −11.11 | 100.28 | 98.12 | 98.71 |
| 305 | 8.31 | 28.32 | 5.54 | 103.57 | 104.75 | 98.83 |
| 306 | 33.06 | 52.20 | 25.31 | 92.86 | 102.01 | 97.59 |
| 307 | 14.59 | 27.42 | 16.64 | 96.52 | 107.89 | 107.90 |
| 308 | 31.92 | 33.29 | 21.49 | 94.54 | 108.68 | 99.95 |
| 309 | 23.20 | 23.91 | 19.42 | 92.83 | 101.62 | 99.54 |
| 310 | 12.01 | 11.79 | 12.55 | 96.22 | 110.59 | 106.09 |
| 311 | 31.19 | 32.15 | 18.08 | 89.34 | 97.90 | 97.62 |
| 312 | 40.71 | 55.74 | 38.57 | 98.42 | 107.25 | 97.64 |
| 313 | 41.41 | 50.97 | 40.33 | 96.41 | 108.12 | 92.66 |
| 314 | 14.01 | 31.41 | −0.78 | 98.21 | 109.88 | 94.95 |
| 315 | 26.26 | 29.33 | 5.07 | 90.61 | 106.80 | 98.26 |
| 316 | 37.08 | 46.50 | 12.96 | 93.74 | 107.11 | 104.14 |
| 317 | 51.78 | 43.32 | 12.22 | 88.27 | 104.56 | 100.58 |
| 318 | 33.47 | 31.48 | 0.53 | 94.93 | 110.68 | 100.29 |
| 319 | 11.31 | 18.81 | −10.01 | 98.24 | 110.36 | 112.52 |
| 320 | 33.29 | 39.26 | 20.22 | 88.29 | 106.49 | 101.75 |
| 321 | −2.96 | 9.62 | 5.34 | 105.21 | 113.09 | 107.79 |
| 322 | 18.68 | 39.85 | 24.22 | 95.12 | 103.05 | 99.04 |
| 323 | −2.15 | 5.60 | 5.09 | 96.44 | 109.51 | 98.75 |
| 324 | 47.44 | 55.26 | 29.99 | 89.90 | 92.43 | 94.77 |
| 325 | 56.54 | 57.44 | 25.20 | 90.87 | 90.49 | 98.42 |
| 326 | 54.63 | 61.27 | 40.35 | 89.15 | 97.52 | 95.73 |
| 327 | 50.64 | 58.86 | 27.99 | 93.07 | 96.65 | 99.67 |
| 328 | 54.26 | 52.11 | 30.99 | 103.06 | 104.25 | 96.28 |
| 329 | 26.55 | 26.12 | 2.44 | 103.91 | 101.51 | 100.06 |
| 330 | 9.37 | 23.31 | 12.48 | 102.16 | 100.90 | 98.13 |
| 331 | 21.72 | 22.05 | 9.94 | 102.31 | 104.94 | 97.11 |
| 332 | 12.40 | 13.59 | −11.55 | 100.05 | 104.66 | 98.42 |
| 333 | 63.01 | 51.36 | 16.76 | 104.07 | 100.30 | 104.80 |
| 334 | 26.69 | 19.54 | 1.94 | 102.83 | 101.42 | 99.88 |
| 335 | 30.37 | 27.46 | −6.52 | 93.88 | 101.43 | 97.67 |
| 336 | 26.45 | 33.43 | 15.13 | 93.86 | 93.51 | 95.37 |
| 337 | 26.22 | 21.56 | 11.07 | 92.21 | 105.02 | 98.08 |
| 338 | 15.56 | 7.76 | −9.44 | 96.78 | 98.57 | 97.30 |
| 339 | 34.37 | 31.12 | 14.82 | 100.11 | 102.71 | 101.95 |
| 340 | 59.51 | 51.71 | 39.14 | 88.92 | 99.63 | 100.50 |
| 341 | 47.82 | 37.03 | 19.96 | 90.58 | 101.85 | 98.94 |
| 342 | 62.68 | 49.22 | 20.96 | 95.56 | 102.43 | 96.82 |
| 343 | 59.88 | 43.35 | 25.09 | 93.46 | 103.94 | 99.58 |
| 344 | 37.96 | 27.38 | 10.37 | 94.16 | 99.37 | 98.71 |
| 345 | −3.84 | 18.41 | −9.62 | 96.05 | 98.00 | 99.35 |
| 346 | 35.46 | 26.05 | −6.00 | 96.12 | 98.96 | 98.83 |
| 347 | 24.91 | 33.00 | −2.81 | 97.47 | 97.41 | 95.44 |
| 348 | 65.80 | 55.42 | 21.00 | 97.61 | 97.89 | 102.76 |
| 349 | −18.52 | −15.49 | −10.48 | 95.53 | 98.66 | 100.43 |
| 350 | 25.78 | 17.55 | −3.37 | 97.15 | 100.08 | 101.89 |
| 351 | 28.09 | 20.40 | 9.27 | 97.92 | 101.17 | 101.97 |
| 352 | 21.89 | 25.05 | −3.63 | 90.30 | 100.84 | 105.31 |
| 353 | −7.60 | −1.19 | −8.35 | 93.24 | 101.22 | 105.10 |
| 354 | 16.19 | 23.11 | −6.55 | 93.49 | 94.82 | 95.57 |
| 355 | −7.88 | −0.33 | −7.55 | 94.65 | 98.80 | 97.25 |
| 356 | 13.33 | 13.26 | −11.92 | 99.34 | 103.85 | 98.17 |
| 357 | −1.55 | −4.03 | −5.62 | 95.33 | 99.62 | 99.17 |
| 358 | 3.46 | −0.23 | −15.71 | 100.23 | 97.59 | 101.83 |
| 359 | 16.19 | −2.46 | −10.76 | 99.70 | 101.80 | 97.95 |
| 360 | 14.52 | 3.54 | 17.38 | 98.78 | 100.55 | 97.87 |
| 361 | 12.02 | 17.57 | −0.65 | 95.21 | 105.08 | 101.48 |
| 362 | −4.38 | 0.74 | −0.49 | 93.09 | 104.40 | 100.92 |
| 363 | 21.62 | 30.44 | 7.08 | 85.34 | 93.98 | 97.70 |
| 364 | 28.72 | 35.24 | 0.54 | 91.16 | 99.18 | 98.75 |
| 365 | 31.66 | 43.95 | 12.03 | 95.03 | 98.16 | 97.03 |
| 366 | 34.71 | 40.19 | 5.72 | 97.43 | 99.89 | 98.90 |
| 367 | 33.60 | 35.22 | 10.12 | 100.04 | 100.33 | 97.63 |
| 368 | 28.94 | 12.52 | 1.16 | 94.33 | 95.19 | 100.37 |
| 369 | 55.35 | 35.43 | 5.93 | 92.30 | 95.88 | 101.72 |
| 370 | 50.43 | 49.81 | 13.28 | 90.56 | 97.40 | 99.78 |
| 371 | 28.97 | 31.48 | −5.99 | 99.16 | 100.36 | 102.50 |
| 372 | 2.62 | −4.89 | −1.96 | 95.84 | 100.26 | 97.50 |
| 373 | −0.08 | 0.30 | −21.78 | 97.68 | 95.79 | 105.68 |
| 374 | −6.80 | 2.06 | 0.31 | 94.66 | 97.48 | 102.35 |
| 375 | −20.26 | −6.85 | −13.67 | 92.00 | 94.62 | 91.64 |
| 376 | −27.64 | −2.00 | −16.87 | 95.42 | 96.79 | 91.71 |
| 377 | −21.57 | −19.05 | −16.76 | 97.45 | 100.30 | 98.60 |
| 378 | −43.00 | −40.96 | −34.24 | 96.85 | 97.50 | 96.03 |
| 379 | −33.27 | −38.38 | −31.21 | 95.89 | 99.21 | 96.91 |
| 380 | −19.77 | −14.96 | −12.33 | 94.08 | 99.77 | 98.38 |
| 381 | −9.00 | −20.39 | −27.54 | 95.76 | 97.56 | 101.71 |
| 382 | −20.63 | −17.93 | −33.16 | 94.93 | 97.94 | 105.20 |
| 383 | −12.55 | −33.45 | −20.39 | 91.48 | 99.48 | 102.78 |
| 384 | 11.98 | 13.93 | −6.54 | 95.98 | 100.98 | 96.43 |
| 385 | −0.45 | 11.66 | 8.33 | 97.86 | 101.23 | 100.14 |
| 386 | 8.41 | 15.56 | 0.44 | 110.65 | 101.21 | 104.15 |
| 387 | 48.91 | 39.09 | 21.49 | 99.70 | 103.20 | 103.29 |
| 388 | 46.76 | 24.20 | −3.90 | 99.26 | 101.05 | 103.31 |
| 389 | 42.99 | 32.83 | 10.15 | 100.03 | 101.93 | 102.81 |
| 390 | 31.39 | 42.36 | −5.37 | 102.52 | 103.04 | 100.79 |
| 391 | 58.92 | 50.04 | 26.86 | 102.89 | 103.14 | 98.70 |
| 392 | 61.86 | 56.97 | 18.83 | 98.41 | 104.40 | 99.38 |
| 393 | 48.05 | 51.00 | 23.93 | 92.22 | 96.99 | 93.89 |
| 394 | 22.57 | 16.81 | 11.21 | 97.85 | 99.70 | 95.68 |
| 395 | 39.19 | 32.15 | −5.97 | 102.16 | 103.84 | 102.47 |
| 396 | 14.01 | 7.28 | 1.11 | 104.22 | 99.84 | 97.85 |
| 397 | 15.64 | 20.08 | −18.09 | 104.06 | 99.85 | 98.78 |
| 398 | 27.09 | 13.83 | −4.58 | 101.69 | 101.50 | 98.48 |
| 399 | 13.71 | 2.77 | −14.45 | 99.12 | 101.60 | 97.93 |
| 400 | −7.11 | −1.40 | −25.16 | 97.90 | 99.49 | 99.38 |
| 401 | 25.46 | 17.81 | 1.69 | 98.31 | 104.27 | 103.33 |
| 402 | 3.18 | 17.64 | 24.96 | 90.37 | 94.75 | 95.87 |
| 403 | 18.58 | 26.80 | 20.48 | 94.49 | 98.99 | 96.32 |
| 404 | 28.02 | 28.76 | 2.60 | 93.50 | 100.33 | 100.23 |
| 405 | 18.82 | 12.31 | 11.23 | 96.26 | 97.10 | 97.80 |
| 406 | 20.60 | 28.67 | 2.45 | 94.48 | 97.54 | 96.24 |
| 407 | 2.95 | 13.84 | 3.90 | 96.60 | 97.86 | 95.80 |
| 408 | 69.13 | 55.88 | 17.43 | 97.18 | 94.56 | 98.79 |
| 409 | 71.13 | 62.34 | 24.05 | 98.79 | 96.70 | 97.35 |
| 410 | 23.77 | 3.56 | 21.75 | 97.25 | 99.40 | 102.21 |
| 411 | 20.52 | 12.82 | 6.93 | 87.10 | 98.34 | 94.89 |
| 412 | 34.68 | 22.44 | 0.91 | 92.77 | 103.32 | 96.15 |
| 413 | −24.52 | 0.42 | 3.41 | 99.95 | 103.89 | 97.97 |
| 414 | 27.62 | 21.32 | 4.58 | 99.90 | 102.35 | 99.00 |
| 415 | −7.34 | −4.53 | −5.81 | 98.73 | 102.54 | 99.93 |
| 416 | 19.68 | 8.93 | −1.82 | 99.89 | 104.57 | 97.95 |
| 417 | −4.66 | 2.52 | −4.37 | 96.87 | 103.47 | 98.89 |
| 418 | 14.52 | 1.38 | 0.36 | 94.77 | 102.91 | 95.45 |
| 419 | 15.11 | 5.52 | −1.31 | 96.51 | 105.30 | 101.36 |
| 420 | 11.09 | 14.17 | 4.56 | 91.49 | 97.42 | 106.30 |
| 421 | −0.18 | 5.44 | −4.15 | 91.14 | 95.41 | 100.05 |
| 422 | 24.55 | 15.03 | 0.83 | 93.76 | 100.22 | 102.43 |
| 423 | 42.05 | 45.83 | 25.09 | 93.64 | 104.07 | 97.48 |
| 424 | 44.81 | 42.34 | 28.35 | 78.76 | 94.74 | 99.68 |
| 425 | 5.45 | 17.81 | −8.31 | 94.40 | 101.15 | 98.94 |
| 426 | 25.87 | 18.04 | 8.28 | 94.17 | 104.08 | 98.96 |
| 427 | 17.76 | 15.06 | 10.31 | 89.83 | 98.20 | 103.13 |
| 428 | 36.82 | 25.44 | 11.61 | 79.00 | 98.77 | 102.07 |
| 429 | 53.36 | 49.45 | 30.57 | 86.64 | 95.84 | 95.62 |
| 430 | 19.00 | 16.76 | 3.52 | 97.98 | 102.63 | 102.36 |
| 431 | 32.65 | 30.01 | 6.99 | 95.33 | 102.80 | 103.28 |
| 432 | −8.29 | 2.15 | −0.99 | 96.49 | 98.19 | 97.00 |
| 433 | 5.02 | −5.87 | −19.44 | 100.56 | 106.26 | 97.91 |
| 434 | 36.19 | 37.23 | 1.44 | 99.88 | 93.08 | 93.61 |
| 435 | −11.27 | −3.48 | −17.19 | 106.42 | 100.65 | 96.45 |
| 436 | 21.09 | 26.67 | −4.13 | 107.31 | 105.14 | 100.39 |
| 437 | −10.63 | −10.54 | 1.73 | 104.60 | 95.31 | 98.01 |
| 438 | −24.85 | 0.48 | −11.85 | 102.03 | 102.66 | 95.76 |
| 439 | −1.12 | −23.39 | −18.88 | 99.18 | 102.47 | 97.70 |
| 440 | −8.09 | −7.33 | −5.48 | 94.22 | 89.62 | 104.04 |
| 441 | 43.66 | 46.52 | 21.28 | 95.64 | 103.05 | 99.09 |
| 442 | 70.00 | 68.51 | 51.65 | 94.03 | 98.36 | 96.73 |
| 443 | −2.27 | −10.41 | −16.58 | 98.78 | 97.22 | 93.45 |
| 444 | −4.33 | 3.62 | 7.61 | 96.38 | 97.11 | 98.10 |
| 445 | 27.13 | 18.78 | −11.78 | 99.09 | 95.26 | 95.54 |
| 446 | 20.40 | 8.71 | 5.78 | 104.55 | 94.63 | 98.83 |
| 447 | 38.22 | 31.08 | 6.55 | 101.43 | 95.00 | 95.42 |
| 448 | −8.18 | 4.46 | 10.04 | 98.70 | 100.47 | 97.35 |
| 449 | −4.38 | 6.19 | 8.06 | 101.23 | 98.25 | 101.39 |
| 450 | 7.89 | 7.25 | −2.08 | 101.66 | 104.87 | 97.01 |

### Differential Gene Expression Assay in Huh-7 Cells

Cell Culture and siRNA Treatment

The ability of a subset of the siRNA sequences disclosed in Table 2 to knockdown the expression of endogenous PNPLA3 in Huh-7 cells, which are homozygous for the rs738409[G](I148M) variant, was determined. Each siRNA molecule tested consisted of a duplex of two siRNA strands, the sense strand and the antisense strand, corresponding to certain siRNA Duplex ID Nos. in Table 2 above.

Hepatoma-derived Huh-7 cells (JCRB Cell Bank, JCRB0403) were routinely cultured in DMEM (Corning, 10-013-CM) supplemented with 10% FBS and 1% P/S at 37° C. and 5% CO₂ until 80-90% confluency. Cells were then detached with 0.05% trypsin (Corning, 25-052-CV), resuspended in fresh DMEM, and seeded into collagen-coated, 96-well microplates. Cells were transfected with serially diluted siRNA and Opti-MEM™ using Lipofectamine RNAiMAX (Invitrogen, 13778100). A mock transfection control, which consisted of transfecting 1×phosphate-buffered saline, was included.

### Cell Lysis and RT-qPCR

After about 48 hours of siRNA treatment, the Huh-7 cells were processed with the TaqMan Fast Advanced Cells-to-Ct Kit (Invitrogen, A35378), according to the manufacturer's protocol. The cell lysates were used for reverse transcription, and the resulting cDNA was diluted 1:2 with nuclease-free, distilled water (Invitrogen, 10977015). Gene expression was measured using TaqMan Fast Advanced Master Mix (Applied Biosystems, 4444964) and the PNPLA3 and ACTB TaqMan Gene Expression assays (Applied Biosystems, 4331182); ACTB served as the endogenous control housekeeping gene. Aliquots of 10 pL were run on the QuantStudio™ 6 Pro Real-Time PCR System (Applied Biosystems) and relative quantification (RQ) of gene expression was calculated via the 2^{-ΔΔCt} method. Gene expression of siRNA wells was normalized to mock wells, percent inhibition was calculated, and dose-response curves were fitted by non-linear regression with variable slope.

Additionally, CellTiter-Glo© Luminescent Cell Viability Assays were also performed with similarly treated Huh-7 cells to assess cytotoxic effects. Assays were performed according to the manufacturer's protocol, and luminescence was measured on an EnVision plate reader. The luminescence from siRNA-treated wells were then normalized to luminescence of mock-treated wells, and percentage viability was calculated.

The results of the RT-qPCR assay and CellTiter-Glo viability assay in Huh-7 cells are provided in Table 4 below.

TABLE 4
RT-qPCR Assay and CellTiter-Glo Viability Assay in Huh-7 Cells

| siRNA | RT-qPCR in Huh-7 | | CellTiter-Glo |
|---|---|---|---|
| Duplex ID | | Maximum % PNPLA3 | in Huh-7 |
| No. (MDx) | EC₅₀ (nM) | RNA inhibition | CC₅₀ (nM) |
| 14 | 0.919 | 68 | >20 |
| 93 | 0.552 | 68 | >20 |
| 95 | 0.256 | 58 | >20 |
| 96 | 0.323 | 69 | >20 |
| 98 | 0.117 | 48 | >20 |
| 99 | 0.243 | 73 | >20 |
| 102 | 0.221 | 69 | >20 |
| 104 | 0.891 | 66 | >20 |
| 105 | 0.580 | 68 | >20 |
| 106 | 0.164 | 78 | >20 |
| 107 | 0.263 | 70 | >20 |
| 108 | 0.194 | 63 | >20 |
| 109 | 0.625 | 61 | >20 |
| 112 | 0.462 | 56 | >20 |
| 115 | — | 55 | >20 |
| 116 | 4.370 | 89 | >20 |
| 119 | 1.260 | 80 | >20 |
| 121 | 0.172 | 80 | >20 |
| 122 | 0.732 | 62 | >20 |
| 124 | 0.028 | 56 | >20 |
| 141 | 0.040 | 63 | >20 |
| 142 | 1.550 | 78 | >20 |
| 143 | 0.046 | 68 | >20 |
| 149 | 0.208 | 51 | >20 |
| 150 | — | 68 | >20 |
| 171 | 0.109 | 68 | >20 |
| 189 | 1.570 | 71 | >20 |
| 218 | — | 37 | >20 |
| 221 | 0.296 | 68 | >20 |
| 222 | 0.382 | 62 | >20 |
| 223 | — | 64 | >20 |
| 227 | 0.025 | 71 | >20 |
| 253 | 0.919 | 70 | >20 |
| 256 | — | 79 | >20 |
| 306 | 0.226 | 50 | >20 |
| 312 | 0.298 | 58 | >20 |
| 313 | 0.310 | 61 | >20 |
| 317 | 1.100 | 55 | >20 |
| 324 | 1.840 | 73 | >20 |
| 325 | 0.679 | 74 | >20 |
| 326 | 2.400 | 56 | >20 |
| 327 | 0.876 | 73 | >20 |
| 328 | 0.148 | 68 | >20 |
| 333 | 0.062 | 69 | >20 |
| 340 | 0.008 | 68 | >20 |
| 342 | 0.029 | 65 | >20 |
| 343 | 0.070 | 77 | >20 |
| 348 | 0.035 | 63 | >20 |
| 369 | 0.153 | 50 | >20 |
| 370 | 0.052 | 70 | >20 |
| 391 | 0.074 | 60 | >20 |
| 392 | 0.588 | 56 | >20 |
| 393 | 2.303 | 51 | >20 |
| 408 | 0.564 | 69 | >20 |
| 409 | 0.176 | 70 | >20 |
| 429 | — | 31 | >20 |
| 442 | 0.252 | 80 | >20 |
| 461 | 0.859 | 63 | >10 |
| 479 | 0.185 | 46 | >10 |
| 463 | 0.247 | 70 | >10 |
| 481 | 0.029 ± 0.038 | 65 ± 4 | >10 |
| 453 | 0.031 ± 0.010 | 45 ± 3 | >10 |
| 471 | 0.010 ± 0.004 | 42 ± 1 | >10 |
| 459 | 0.047 ± 0.031 | 64 ± 8 | >10 |
| 477 | 0.014 ± 0.005 | 65 ± 8 | >10 |
| 458 | 2.144 | / | >10 |
| 476 | 0.554 | 65 | >10 |
| 454 | 0.128 | 72 | >10 |
| 472 | 0.034 ± 0.042 | 73 ± 6 | >10 |
| 462 | 0.224 | 50 | >10 |
| 480 | 0.116 | 51 | >10 |
| 455 | 0.267 | 43 | >10 |
| 473 | 0.106 | 45 | >10 |
| 467 | 0.090 ± 0.074 | 69 ± 7 | >10 |
| 485 | 0.044 ± 0.039 | 73 ± 3 | >10 |
| 466 | 0.064 | 80 | >10 |
| 484 | 0.012 | 80 | >10 |
| 468 | 0.123 | 67 | >10 |
| 486 | 0.040 | 76 | >10 |
| 452 | 0.034 ± 0.004 | 62 ± 12 | >10 |
| 470 | 0.012 ± 0.006 | 61 ± 3 | >10 |
| 451 | 0.013 ± 0.003 | 52 ± 1 | >10 |
| 469 | 0.037 ± 0.027 | 52 ± 8 | >10 |
| 456 | 0.074 ± 0.081 | 69 ± 6 | >10 |
| 474 | 0.012 | 71 ± 4 | >10 |
| 457 | 0.318 | 57 | >10 |
| 475 | 0.027 | 44 | >10 |
| 460 | 0.035 ± 0.043 | 76 ± 7 | >10 |
| 478 | 0.001 ± 0.001 | 72 ± 7 | >10 |
| 465 | 0.042 | 73 | >10 |
| 483 | 0.033 | 77 | >10 |
| 464 | 0.031 | 63 | >10 |
| 482 | 0.017 | 61 | >10 |
| 535 | 0.053 | 72 | >10 |
| 547 | 0.104 | 83 | >10 |
| 536 | 0.013 | 59 | >10 |
| 548 | 0.012 | 55 | >10 |
| 537 | 0.028 | 69 | >10 |
| 549 | 0.012 | 69 | >10 |
| 538 | 0.062 | 64 | >10 |
| 550 | 0.001 | 66 | >10 |
| 539 | 0.065 | 74 | >10 |
| 551 | 0.007 | 72 | >10 |
| 540 | 0.010 | 79 | >10 |
| 552 | 0.003 | 82 | >10 |
| 541 | 0.009 | 53 | >10 |
| 553 | 0.001 | 46 | >10 |
| 542 | 0.018 | 56 | >10 |
| 554 | 0.003 | 49 | >10 |
| 543 | 0.162 | 67 | >10 |
| 555 | 0.010 | 61 | >10 |
| 544 | 0.012 | 70 | >10 |
| 556 | 0.014 | 72 | >10 |
| 545 | 0.153 | 72 | >10 |
| 557 | 0.072 | 62 | >10 |
| 546 | 0.335 | 80 | >10 |
| 558 | 0.157 | 80 | >10 |
| 559 | 0.923 | 52 | >10 |
| 571 | 0.227 | 67 | >10 |
| 560 | 0.034 | 39 | >10 |
| 572 | 0.140 | 46 | >10 |
| 561 | 0.153 | 67 | >10 |
| 573 | 0.101 | 62 | >10 |
| 562 | 0.054 | 65 | >10 |
| 574 | 0.008 | 73 | >10 |
| 563 | 0.117 | 71 | >10 |
| 575 | 0.013 | 75 | >10 |
| 564 | 0.200 | 79 | >10 |
| 576 | 0.062 | 76 | >10 |
| 565 | 0.019 | 59 | >10 |
| 577 | 0.003 | 59 | >10 |
| 566 | 0.458 | 68 | >10 |
| 578 | 0.008 | 63 | >10 |
| 567 | 0.152 | 56 | >10 |
| 579 | 0.129 | 50 | >10 |
| 568 | 0.731 | 79 | >10 |
| 580 | 0.031 | 61 | >10 |
| 569 | 0.850 | 36 | >10 |
| 581 | 0.210 | 37 | >10 |
| 570 | 0.756 | 80 | >10 |
| 582 | 0.401 | 80 | >10 |

### Example 61: In Vivo Effect Single Dose Administration of siRNA Molecules in a Mouse Model

Certain siRNA molecules were selected for initial pharmacokinetic/pharmacodynamic studies in vivo. To enhance targeted delivery to hepatocytes, a GalNAc ligand was incorporated at the 3′ end of the sense strand via standard phosphoramidite chemistry. The specific GalNAc ligand used (“GalNAc4-ps-GalNAc4-ps GalNAc4” or “p-(ps)₂-GalNAc4”), shown below, includes three monomeric “GalNAc4” derivative units linked through two phosphorothioate linkages, where one GalNAc4 unit is linked to the 3′ end nucleotide on the sense strand via a standard phosphodiester linkage.

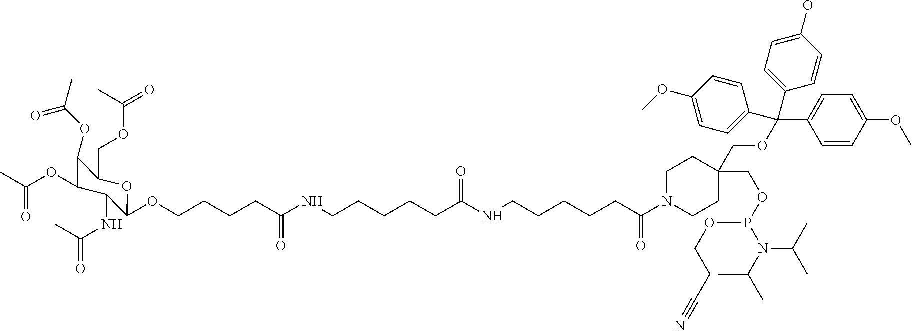 [SMILES: COc1ccc(C(OCC2(COP(OCCC#N)N(C(C)C)C(C)C)CCN(C(=O)CCCCCNC(=O)CCCCCNC(=O)CCCCOC3OC(COC(C)=O)C(OC(C)=O)C(OC(C)=O)C3NC(C)=O)CC2)(c2ccc(O)cc2)c2ccc(OC)cc2)cc1]

### Structure of “Monomeric GalNAc4 Phosphoramidite”

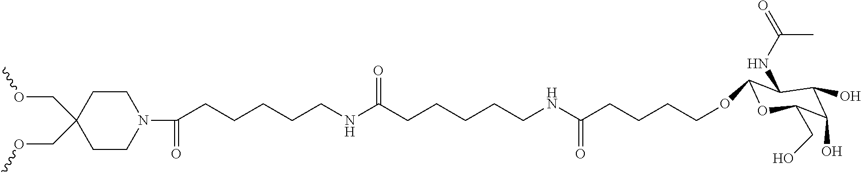

### Structure of “Monomeric GalNAc4”

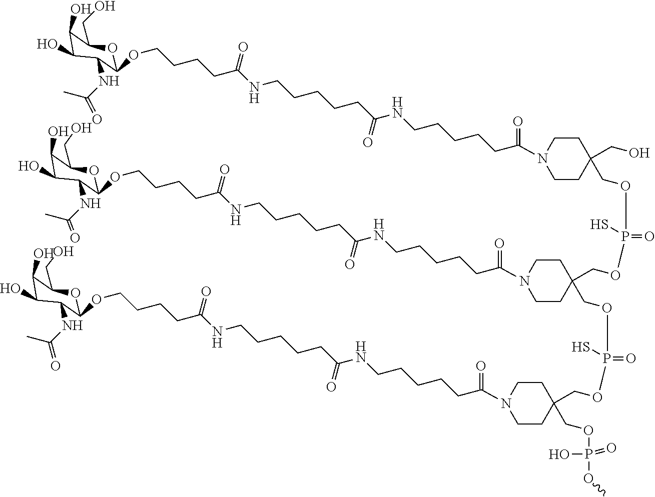

### Structure of “GalNAc4-ps-GalNAc4-ps GalNAc4” or “p-(ps)₂-GalNAc4”

A C57BL/6 human PNPLA3-knock-in (hPNPLA3-KI) mouse model, which expresses a human PNPLA3 insert, was used for in vivo PK/PD studies. On Day 0, 2-month-old mice were administered either a single subcutaneous (SC) dose of a GalNAc-conjugated siRNA duplex or a no-drug vehicle (n=5 animals per group in each of seven groups). The animals were sacrificed on Day 4 (about 96 hours post-dose). The right, lateral liver lobe of each animal was collected for RT-qPCR and the left, lateral liver lobe was collected for PK analysis. RT-qPCR was performed to measure levels of human PNPLA3 expression.

For RT-qPCR, RNA was extracted using the RNeasy Mini Kit (Qiagen, 74106), according to the manufacturer's protocol. RNA quantity and quality was analyzed with a NanoDrop™ Lite Spectrophotometer (Thermo Scientific), and cDNA was synthesized using the SuperScript IV VILO Master Mix (Invitrogen, 11756500), according to the manufacturer's protocol. Gene expression was measured using TaqMan Fast Advanced Master Mix (Applied Biosystems, 4444964), and the following TaqMan Gene Expression assays (Applied Biosystems, 4331182): Actb (Mm00607939_s1) and PNPLA3 (Hs00228747_ml). Actb served as the endogenous control gene. RT-qPCR reactions were run on the QuantStudio™ 6 Pro Real-Time PCR System (Applied Biosystems). The RQ of gene expression was calculated via the 2^{-ΔΔCt} (RQ) method. Results are presented as expression relative to the expression levels of vehicle control samples.

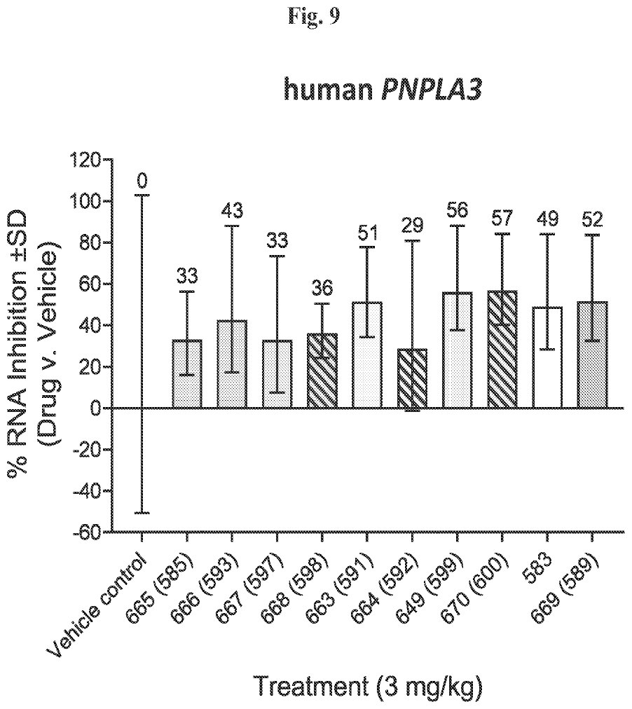

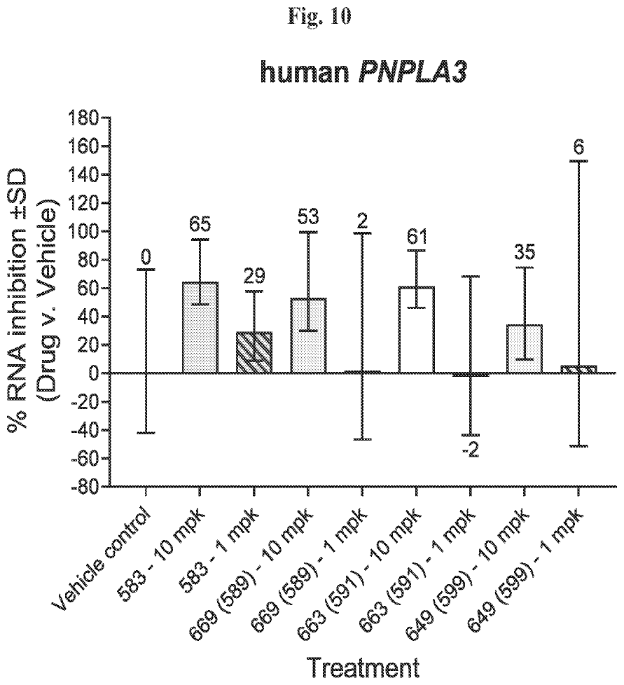

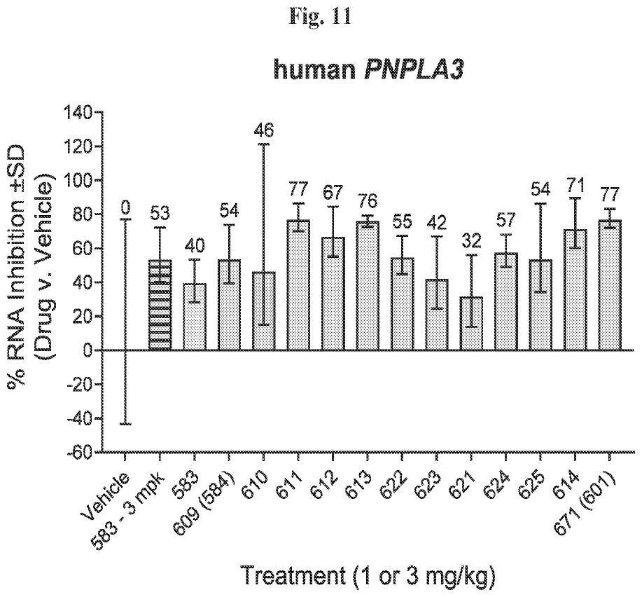

In one study, the GalNAc-conjugated siRNA duplexes of Table 5 or a no-drug vehicle were tested in accordance with the procedure outlined above at 1 mg/kg, 3 mg/kg and/or 10 mg/kg. The results are shown in Table 5 and FIGS. 9-11.

TABLE 5
Modified siRNA Sequences Used for Further In Vivo Study

| siRNA | | | | | | | |
|---|---|---|---|---|---|---|---|
| Duplex ID | SEQ ID | Sense Strand Base Sequence + | SEQ ID | Antisense Strand Base Sequence + | %RNA Inhibition (Drug v. Vehicle) | | |
| No. (MDx) | NO | Modifications (5′-3′) | NO | Modifications (5′-3′) | 1 mg/kg | 3 mg/kg | 10 mg/kg |
| 665(585) | 2293 | mApsmUpsmGmGfAmGfGfAfGmUm | 2317 | mUpsfUpsmGmUmCfAmCmUmCmA | | 33 | |
| | | GmAmGmUmGmAmCmAmA-p- | | mCmUmCfCmUfCmCmAmUpsmUpsm | | | |
| | | (ps)2-GalNAc4 | | U | | | |
| 666(593) | 2294 | mApsmUpsmGmCmCmAfAmAfAfCfA | 2318 | mCpsfGpsmGmUmGfAmUmGmGmU | | 43 | |
| | | mAmCmCmAmUmCmAmCmCmG-p- | | mUmGmUfUmUfUmGmGmCmAmUp | | | |
| | | (ps)2-GalNAc4 | | smUpsmU | | | |
| 667(597) | 2295 | mGpsmApsmAmGfUmCmGmUfGfGf | 2319 | mUpsfApsmCmCmAmAmGmGmCmA | | 33 | |
| | | AmUmGfCmCmUmUmGfGmUmA-p- | | mUmCmCfAmCmGmAmCmUmUmCp | | | |
| | | (ps)2-GalNAc4 | | smUpsmU | | | |
| 668(598) | 2296 | mGpsmApsmAmGfUmCmGmUfGfGf | 2320 | vmUpsfApsmCmCmAmAmGmGmCm | | 36 | |
| | | AmUmGfCmCmUmUmGfGmUmA-p- | | AmUmCmCfAmCmGmAmCmUmUmC | | | |
| | | (ps)2-GalNAc4 | | psmUpsmU | | | |
| 663(591) | 2186 | mUpsmCpsmGmUmGmGfAmUfGfCf | 2024 | mApsfApsmCmAmUfAmCmCmAmAm | −2 | 51 | 61 |
| | | CmUmUmGmGmUmAmUmGmUmU- | | GmGmCfAmUfCmCmAmCmGmApsm | | | |
| | | p-(ps)2-GalNAc4 | | UpsmU | | | |
| 664(592) | 2292 | mUpsmCpsmGmUmGmGfAmUfGfCf | 2316 | vmApsfApsmCmAmUfAmCmCmAmA | | 29 | |
| | | CmUmUmGmGmUmAmUmGmUmU- | | mGmGmCfAmUfCmCmAmCmGmAps | | | |
| | | p-(ps)2-GalNAc4 | | mUpsmU | | | |
| 649(599) | 2278 | mUpsmCpsmCmAmAmAfGmAfCfGf | 2302 | mApsfUpsmCmCmAfCmGmAmCmUm | 6 | 56 | 35 |
| | | AmAmGmUmCmGmUmGmGmAmU- | | UmCmGfUmCfUmUmUmGmGmApsm | | | |
| | | p-(ps)2-GalNAc4 | | UpsmU | | | |
| 670(600) | 2298 | mUpsmCpsmCmAmAmAfGmAfCfGf | 2322 | vmApsfUpsmCmCmAfCmGmAmCmU | | 57 | |
| | | AmAmGmUmCmGmUmGmGmAmU- | | mUmCmGfUmCfUmUmUmGmGmAps | | | |
| | | p-(ps)2-GalNAc4 | | mUpsmU | | | |
| 583 | 2148 | mApsmUpsmGmCfCmUfUfGfGmUm | 1936 | mCpsfApsmGmGmAfAmCmAmUmAm | 35* | 51* | 65 |
| | | AmUmGmUmUmCmCmUmG-p- | | CmCmAfAmGfGmCmAmUpsmUpsmU | | | |
| | | (ps)2-GalNAc4 | | | | | |
| 669(589) | 2297 | mGpsmUpsmGmUmCmUfGmAfCfUf | 2321 | mUpsfUpsmUmGmGfAmCmCmGmA | 2 | 52 | 53 |
| | | UmUmCmGmGmUmCmCmAmAmA- | | mAmAmGfUmCfAmGmAmCmAmCps | | | |
| | | p-(ps)2-GalNAc4 | | mUpsmU | | | |
| 609(584) | 2148 | mApsmUpsmGmCfCmUfUfGfGmUm | 2188 | vmCpsfApsmGmGmAfAmCmAmUmA | 54 | | |
| | | AmUmGmUmUmCmCmUmG- | | mCmCmAfAmGfGmCmAmUpsmUpsm | | | |
| | | GalNAc4psGalNAc4psGalNAc4 | | U | | | |
| 610 | 2149 | mApsmUpsmGmCfCmUfUfGfGmUm | 2189 | mApsfApsmGmGmAfAmCmAmUmAm | 46 | | |
| | | AmUmGmUmUmCmCmUmG-p- | | CmCmAfAmGfGmCmAmUpsmUpsmU | | | |
| | | (ps)2-GalNAc4 | | | | | |
| 611 | 2150 | mApsmUpsmGmCfCmUfUfGfGmUm | 2190 | vmApsfApsmGmGmAfAmCmAmUmA | 77 | | |
| | | AmUmGmUmUmCmCmUmG- | | mCmCmAfAmGfGmCmAmUpsmUpsm | | | |
| | | GalNAc4psGalNAc4psGalNAc4 | | U | | | |
| 612 | 2151 | mApsmUpsmGmCfCmUfUfGfGmUm | 2191 | mUpsfApsmGmGmAfAmCmAmUmA | 67 | | |
| | | AmUmGmUmUmCmCmUmG-p- | | mCmCmAfAmGfGmCmAmUpsmUpsm | | | |
| | | (ps)2-GalNAc4 | | U | | | |
| 613 | 2152 | mApsmUpsmGmCfCmUfUfGfGmUm | 2192 | vmUpsfApsmGmGmAfAmCmAmUmA | 76 | | |
| | | AmUmGmUmUmCmCmUmG-p- | | mCmCmAfAmGfGmCmAmUpsmUpsm | | | |
| | | (ps)2-GalNAc4 | | U | | | |
| 622 | 2161 | GalNAc4psGalNAc4psGalNAc4- | 2201 | mCpsfApsmGmGmAfAmCmAmUmAm | 55 | | |
| | | mAmUmGmCfCmUfUfGfGmUmAmU | | CmCmAfAmGfGmCmAmUpsmUpsmU | | | |
| | | mGmUmUmCmCmUmG- | | | | | |
| | | GalNAc4psGalNAc4psGalNAc4 | | | | | |
| 623 | 2162 | mApsmUpsmGmCfCmUfUfGfGmUm | 2202 | mCpsfApsmGmGmAfAmCmAmUmAm | 42 | | |
| | | AmUmGmUmUmCmCmUmG- | | CmCmAfAmGfGmCmAmUpsmUpsmU | | | |
| | | GalNAc4psGalNAc4psGalNAc4ps | | | | | |
| | | GalNAc4 | | | | | |
| 621 | 2160 | mApsmUpsmGmCfCmUfUfGfGmUm | 2200 | mCpsfApsmGmGfAmAmCfAmUmAmC | 32 | | |
| | | AmUmGmUmUmCmCmUmG- | | mCmAfAmGmGfCmAmUpsmUpsmU | | | |
| | | GalNAc4psGalNAc4psGalNAc4 | | | | | |
| 624 | 2163 | mApsmUpsmGmCf2PmUfUfGfGmU | 2203 | mCpsfApsmGmGmAfAmCmAmUmAm | 57 | | |
| | | mAmUmGmUmUmCmCmUmG- | | CmCmAfAmGfGmCmAmUpsmUpsmU | | | |
| | | GalNAc4psGalNAc4psGalNAc4 | | | | | |
| 625 | 2164 | mApsmUpsmGmCfCmUfUfGfGmUm | 2204 | mCpsfApsmGmGmAfAmCmAmUmAm | 54 | | |
| | | AmUmGmUmUmCmun34CmUmG- | | CmCmAfAmGfGmCmAmUpsmUpsmU | | | |
| | | GalNAc4psGalNAc4psGalNAc4 | | | | | |
| 614 | 2153 | mApsmUpsmGmCfCmUfUfGfGmUm | 2193 | mCpsfApsmGmGmAfAmCmAmUmAm | 71 | | |
| | | AmUmGmUmUmCmCmUmG-p- | | CmCmAfAmGfGmCmAmUpsmCpsmC | | | |
| | | (ps)2-GalNAc4 | | | | | |
| 671(601) | 2299 | mGpsmUpsmCmGmUmGfGmAfUfGf | 2323 | mApsfCpsmAmUmAfCmCmAmAmGm | 77 | | |
| | | CmCmUmUmGmGmUmAmUmGmU- | | GmCmAfUmCfCmAmCmGmAmCpsm | | | |
| | | p-(ps)2-GalNAc4 | | UpsmU | | | |

*indicates average value over more than one trial

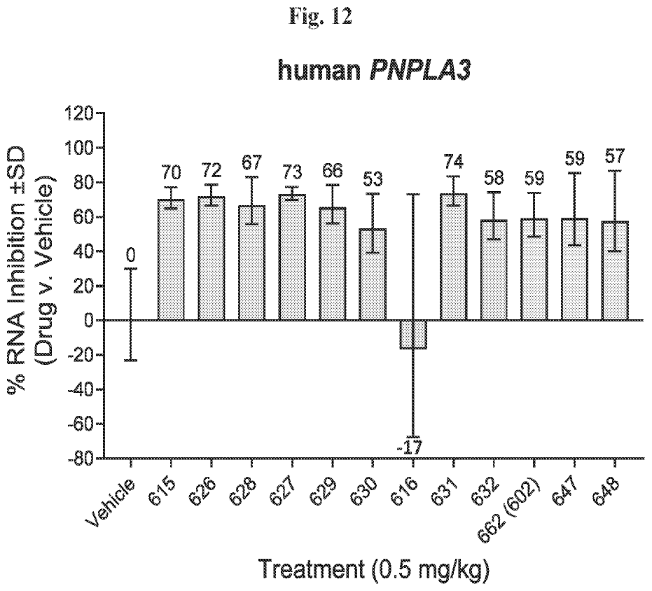

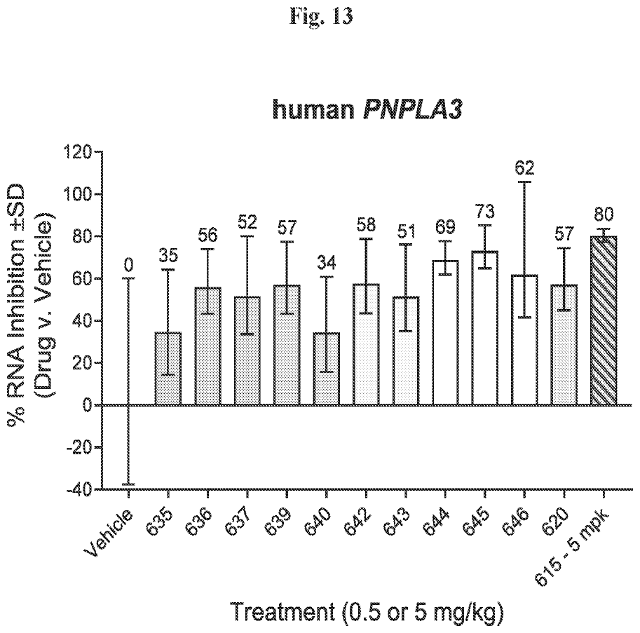

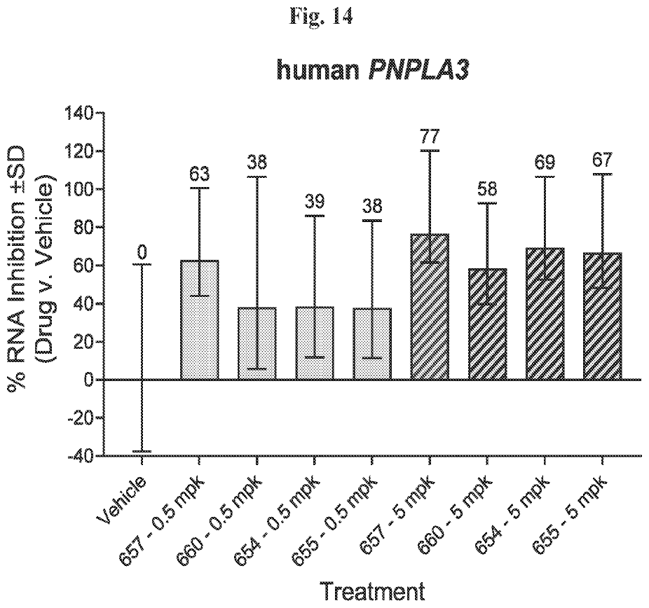

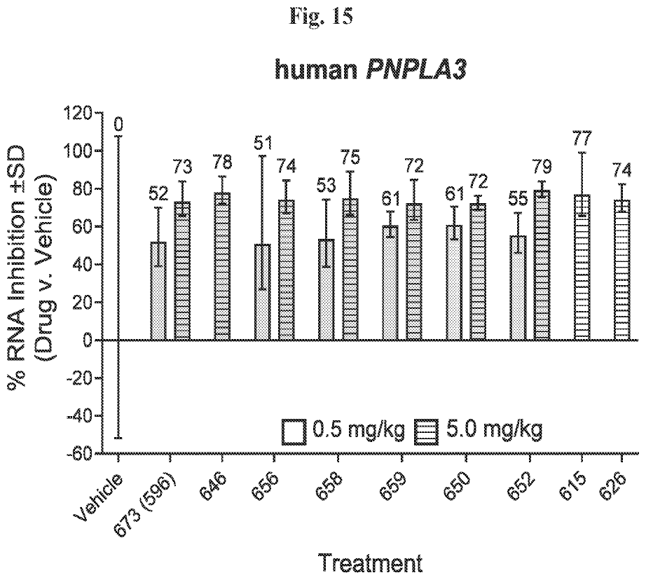

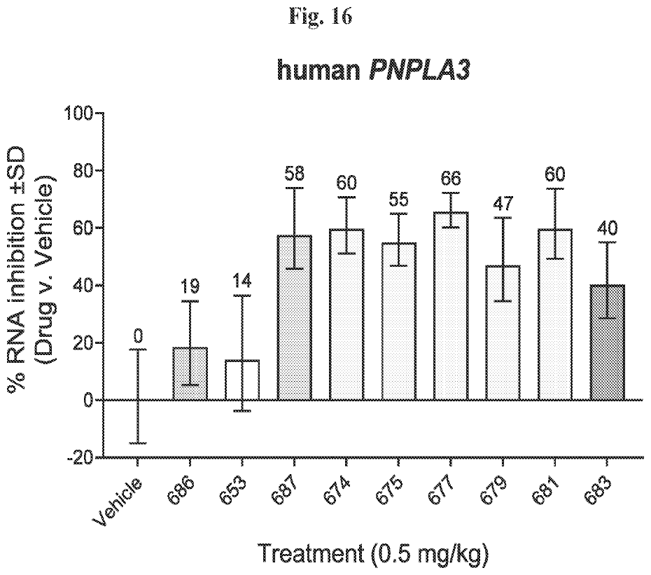

Still additional modified siRNA duplexes were tested at a dose of 0.5 mg/kg or 5 mg/kg in accordance with the procedure outlined above, except that treatment was on Day 0 and the animals were sacrificed on Day 10 (240 hours post-dose). The results are shown in Table 6 and FIGS. 12-16.

TABLE 6
Modified siRNA Sequences Used for Further In Vivo Study

| siRNA | | | | | | |
|---|---|---|---|---|---|---|
| Duplex ID | SEQ | Sense Strand Base Sequence + | SEQ | Antisense Strand Base Sequence + | %RNA Inhibition (Drug v. Vehicle) | |
| No. (MDx) | ID NO | Modifications (5′-3′) | ID NO | Modifications (5′-3′) | 0.5 mg/kg | 5 mg/kg |
| 615 | 2154 | mApsmUpsmGmCfCmUfUfGfGmUmA | 2194 | mApsfApsmGmGmAfAmCmAmUmAmC | 70 | 79* |
| | | mUmGmUmUmCmCmUmG-p-(ps)2- | | mCmAfAmGfGmCmAmUpsmCpsmC | | |
| | | GalNAc4 | | | | |
| 626 | 2165 | mApsmUpsmGmCfCmUfUfGfGmUmA | 2205 | d2vmApsfApsmGmGmAfAmCmAmUmA | 72 | 74 |
| | | mUmGmUmUmCmCmUmG- | | mCmCmAfAmGfGmCmAmUpsmCpsmC | | |
| | | GalNAc4psGalNAc4psGalNAc4 | | | | |
| 628 | 2167 | mApsmUpsmGmCf2PmUfUfGfGmUm | 2207 | d2vmApsfApsmGmGmAfAmCmAmUmA | 67 | |
| | | AmUmGmUmUmCmCmUmG- | | mCmCmAfAmGfGmCmAmUpsmCpsmC | | |
| | | GalNAc4psGalNAc4psGalNAc4 | | | | |
| 627 | 2166 | mApsmUpsmGmCf2PmUfUfGfGmUm | 2206 | mApsfApsmGmGmAfAmCmAmUmAmC | 73 | |
| | | AmUmGmUmUmCmCmUmG- | | mCmAfAmGfGmCmAmUpsmCpsmC | | |
| | | GalNAc4psGalNAc4psGalNAc4 | | | | |
| 629 | 2168 | mApsmUpsmGmCfCmUfUfGfGmUmA | 2208 | d2vmApsfApsmGmGmAfAmCmAmUmA | 66 | |
| | | mUmGmUmUmCmun34CmUmG-p- | | mCmCmAfAmGfGmCmAmUpsmCpsmC | | |
| | | (ps)2-GalNAc4 | | | | |
| 630 | 2169 | GalNAc4-(ps)2-p- | 2209 | mApsfApsmGmGmAfAmCmAmUmAmC | 53 | |
| | | mAmUmGmCfCmUfUfGfGmUmAmU | | mCmAfAmGfGmCmAmUpsmCpsmC | | |
| | | mGmUmUmCmCmUmG-p-(ps)2- | | | | |
| | | GalNAc4 | | | | |
| 616 | 2155 | mApsmUpsmGmCmCmUfUmGfGfUfA | 2195 | mApsfGpsmCmAmGfGmAmAmCmAmU | −17 | |
| | | mUmGmUmUmCmCmUmGmCmU-p- | | mAmCfCmAfAmGmGmCmAmUpsmCps | | |
| | | (ps)2-GalNAc4 | | mC | | |
| 631 | 2170 | mApsmUpsmGmCfCmUfUfGfGmUmA | 2210 | c20- | 74 | |
| | | mUmGmUmUmCmCmUmG-p-(ps)2- | | 4hUpsfApsmGmGmAfAmCmAmUmAmC | | |
| | | GalNAc4 | | mCmAfAmGfGmCmAmUpsmCpsmC | | |
| 632 | 2171 | mGpsmGpsmAmUmGmCfCmUfUfGfG | 2211 | mApsfApsmGmGmAfAmCmAmUmAmC | 58 | |
| | | mUmAmUmGmUmUmCmCmUmG-p- | | mCmAfAmGfGmCmAmUmCmCpsmAps | | |
| | | (ps)2-GalNAc4 | | mC | | |
| 662(602) | 2291 | mGpsmUpsmCmGmUmGfGmAfUfGfC | 2315 | vmApsfCpsmAmUmAfCmCmAmAmGm | 59 | |
| | | mCmUmUmGmGmUmAmUmGmU-p- | | GmCmAfUmCfCmAmCmGmAmCpsmUp | | |
| | | (ps)2-GalNAc4 | | smU | | |
| 647 | 2186 | mUpsmCpsmGmUmGmGfAmUfGfCfC | 2226 | mApsfApsmCmAmUfAmCmCmAmAmG | 59 | |
| | | mUmUmGmGmUmAmUmGmUmU-p- | | mGmCfAmUfCmCmAmCmGmApsmCps | | |
| | | (ps)2-GalNAc4 | | mU | | |
| 648 | 2187 | mApsmGpsmUmCmGmUfGmGfAfUfG | 2227 | mApsfApsmUmAmCfCmAmAmGmGmC | 57 | |
| | | mCmCmUmUmGmGmUmAmUmG-p- | | mAmUfCmCfAmCmGmAmCmUpsmUps | | |
| | | (ps)2-GalNAc4 | | mC | | |
| 635 | 2174 | mGpsmUpsmGmUmCmUfGmAfCfUfU | 2214 | vmUpsfUpsmUmGmGfAmCmCmGmAm | 35 | |
| | | mUmCmGmGmUmCmCmAmAmA-p- | | AmAmGfUmCfAmGmAmCmAmCpsmCp | | |
| | | (ps)2-GalNAc4 | | smA | | |
| 636 | 2175 | mGpsmUpsmGmUmCmUfGmAfCfUfU | 2215 | vmApsfUpsmUmGmGfAmCmCmGmAm | 56 | |
| | | mUmCmGmGmUmCmCmAmAmA-p- | | AmAmGfUmCfAmGmAmCmAmCpsmCp | | |
| | | (ps)2-GalNAc4 | | smA | | |
| 637 | 2176 | mGpsmUpsmGmUmCmUfGmAfCfUfU | 2216 | mApsfUpsmUmGmGfAmCmCmGmAmA | 52 | |
| | | mUmCmGmGmUmCmCmAmAmA-p- | | mAmGfUmCfAmGmAmCmAmCpsmCps | | |
| | | (ps)2-GalNAc4 | | mA | | |
| 639 | 2178 | mUpsmCpsmCmAmAmAfGmAfCfGfA | 2218 | vmUpsfUpsmCmCmAfCmGmAmCmUm | 57 | |
| | | mAmGmUmCmGmUmGmGmAmU-p- | | UmCmGfUmCfUmUmUmGmGmApsmC | | |
| | | (ps)2-GalNAc4 | | psmC | | |
| 640 | 2179 | mUpsmCpsmCmAmAmAfGmAfCfGfA | 2219 | mUpsfUpsmCmCmAfCmGmAmCmUmU | 34 | |
| | | mAmGmUmCmGmUmGmGmAmU-p- | | mCmGfUmCfUmUmUmGmGmApsmCps | | |
| | | (ps)2-GalNAc4 | | mC | | |
| 642 | 2181 | mApsmUpsmGmCmCmAfAmAfAfCfA | 2221 | vmApsfGpsmGmUmGfAmUmGmGmUm | 58 | |
| | | mAmCmCmAmUmCmAmCmCmG-p- | | UmGmUfUmUfUmGmGmCmAmUpsmC | | |
| | | (ps)2-GalNAc4 | | psmA | | |
| 643 | 2182 | mApsmUpsmGmCmCmAfAmAfAfCfA | 2222 | mApsfGpsmGmUmGfAmUmGmGmUm | 51 | |
| | | mAmCmCmAmUmCmAmCmCmG-p- | | UmGmUfUmUfUmGmGmCmAmUpsmC | | |
| | | (ps)2-GalNAc4 | | psmA | | |
| 644 | 2183 | mGpsmApsmAmGmUmCfGmUfGfGfA | 2223 | vmUpsfApsmCmCmAfAmGmGmCmAm | 69 | |
| | | mUmGmCmCmUmUmGmGmUmA-p- | | UmCmCfAmCfGmAmCmUmUmCpsmGp | | |
| | | (ps)2-GalNAc4 | | smU | | |
| 645 | 2184 | mGpsmApsmAmGmUmCfGmUfGfGfA | 2224 | vmApsfApsmCmCmAfAmGmGmCmAm | 73 | |
| | | mUmGmCmCmUmUmGmGmUmA-p- | | UmCmCfAmCfGmAmCmUmUmCpsmGp | | |
| | | (ps)2-GalNAc4 | | smU | | |
| 646 | 2185 | mGpsmApsmAmGmUmCfGmUfGfGfA | 2225 | mApsfApsmCmCmAfAmGmGmCmAmU | 62 | 78 |
| | | mUmGmCmCmUmUmGmGmUmA-p- | | mCmCfAmCfGmAmCmUmUmCpsmGps | | |
| | | (ps)2-GalNAc4 | | mU | | |
| 620 | 2159 | mApsmUpsmGmCfCmUfUfGfGmUmU | 2199 | d2vmApsfApsmGmGmAfAmCmAmAmA | 57 | |
| | | mUmGmUmUmCmCmUmG-p-(ps)2- | | mCmCmAfAmGfGmCmAmUpsmCpsmC | | |
| | | GalNAc4 | | | | |
| 657 | 2286 | mGpsmApsmAmGmUmCfGmUfGfGfC | 2310 | vmApsfApsmCmCmAfAmGmGmCmAm | 63 | 77 |
| | | mUmGmCmCmUmUmGmGmUmA-p- | | UmCmCfAmCfGmAmCmUmUmCpsmGp | | |
| | | (ps)2-GalNAc4 | | smU | | |
| 660 | 2289 | mGpsmApsmAmGmUmCfGmUfGfGfC | 2313 | mApsfApsmCmCmAfAmGmGmCmAmU | 38 | 58 |
| | | mUmGmCmCmUmUmGmGmUmA-p- | | mCmCfAmCfGmAmCmUmUmCpsmGps | | |
| | | (ps)2-GalNAc4 | | mU | | |
| 654 | 2283 | mGpsmApsmAmGmUmCfGmUfGfGfA | 2307 | c20- | 39 | 69 |
| | | mUmGmCmCmUmUmGmGmUmA-p- | | 4hUpsfApsmCmCmAfAmGmGmCmAmU | | |
| | | (ps)2-GalNAc4 | | mCmCfAmCfGmAmCmUmUmCpsmGps | | |
| | | | | mU | | |
| 655 | 2284 | mGpsmApsmAmGmUmCfGmUfGfGfA | 2308 | vmApsfApsmCmCmAfAunGmGmCmAm | 38 | 67 |
| | | mUmGmCmCmUmUmGmGmUmA-p- | | UmCmCfAmCfGmAmCmUmUmCpsmGp | | |
| | | (ps)2-GalNAc4 | | smU | | |
| 673(596) | 2301 | mGpsmApsmAmGmUmCfGmUfGfGfA | 2325 | vmUpsfApsmCmCmAfAmGmGmCmAm | 52 | 73 |
| | | mUmGmCmCmUmUmGmGmUmA-p- | | UmCmCfAmCfGmAmCmUmUmCpsmUp | | |
| | | (ps)2-GalNAc4 | | smU | | |
| 656 | 2285 | mGpsmApsmAmGmUmCfGmUfGfGfA | 2309 | vmApsfApsmCmCmAfAmGmGmCmAm | 51 | 74 |
| | | mUmGmCmCmUmUmGmGmUmU-p- | | UmCmCfAmCfGmAmCmUmUmCpsmGp | | |
| | | (ps)2-GalNAc4 | | smU | | |
| 658 | 2287 | mGpsmApsmAmGmUmCfGmUfGfGfA | 2311 | vmApsfApsmCmCmAfAmGmGmCmAm | 53 | 75 |
| | | mUmGmCmCmUmUmGmun34GmU | | UmCmCfAmCfGmAmCmUmUmCpsmGp | | |
| | | mA-p-(ps)2-GalNAc4 | | smU | | |
| 659 | 2288 | mGpsmApsmAmGmUmCfGmUfGfGfA | 2312 | mApsfApsmCmCmAfAmGmGmCmAmU | 61 | 72 |
| | | mUmGmCmCmUmUmGmGmUmU-p- | | mCmCfAmCfGmAmCmUmUmCpsmGps | | |
| | | (ps)2-GalNAc4 | | mU | | |
| 650 | 2279 | mApsmUpsmGmCmCmAfAmAfAf2PfA | 2303 | vmApsfGpsmGmUmGfAmUmGmGmUm | 61 | 72 |
| | | mAmCmCmAmUmCmAmCmCmG-p- | | UmGmUfUmUfUmGmGmCmAmUpsmC | | |
| | | (ps)2-GalNAc4 | | psmA | | |
| 652 | 2281 | mApsmUpsmGmCmCmAfAmAfAfCfA | 2305 | vmApsfGpsmGmUmGfAmUmGmGmUm | 55 | 79 |
| | | mAmCmCmAmUmCmAmCmCmU-p- | | UmGmUfUmUfUmGmGmCmAmUpsmC | | |
| | | (ps)2-GalNAc4 | | psmA | | |
| 686 | 2338 | mUpsmApsmCmCmAmGfAmGfUfGfU | 2352 | vmUpsfCpsmCmCmCfAmUmCmAmGmA | 19 | |
| | | mCmUmGmAmUmGmGmGmGmA-p- | | mCmAfCmUfCmUmGmGmUmApsmAps | | |
| | | (ps)2GalNAc | | mG | | |
| 653 | 2282 | mApsmUpsmGmCmCmAfAmAfAf2PfA | 2306 | vmApsfGpsmGmUmGfAmUmGmGmUm | 14 | |
| | | mAmCmCmAmUmCmAmCmCmU-p- | | UmGmUfUmUfUmGmGmCmAmUpsmC | | |
| | | (ps)2-GalNAc4 | | psmA | | |
| 687 | 2339 | mCpsmGpsmAmCmAmUfCmUfGfCfC | 2353 | vmUpsfUpsmGmAmCfUmUmUmAmGm | 58 | |
| | | mCmUmAmAmAmGmUmCmAmA-p- | | GmGmCfAmGfAmUmGmUmCmGpsmU | | |
| | | (ps)2GalNAc | | psmA | | |
| 674 | 2326 | mApsmCpsmAmUfCmUfGfCfCmCmU | 2340 | mUpsfUpsmGmAmCfUmUmUmAmGmG | 60 | |
| | | mAmAmAmGmUmCmAmA-p-(ps)2- | | mGmCfAmGfAmUmGmUpsmCpsmG | | |
| | | GalNAc4 | | | | |
| 675 | 2327 | mApsmCpsmAmUfCmUfGfCfCmCmU | 2341 | vmUpsfUpsmGmAmCfUmUmUmAmGm | 55 | |
| | | mAmAmAmGmUmCmAmA-p-(ps)2- | | GmGmCfAmGfAmUmGmUpsmCpsmG | | |
| | | GalNAc4 | | | | |
| 677 | 2329 | mApsmCpsmAmUfCmUfGfCfCmCmU | 2343 | vmApsfUpsmGmAmCfUmUmUmAmGm | 66 | |
| | | mAmAmAmGmUmCmAmA-p-(ps)2- | | GmGmCfAmGfAmUmGmUpsmCpsmG | | |
| | | GalNAc4 | | | | |
| 679 | 2331 | mApsmCpsmAmUf2PmUfGfCfCmCmU | 2345 | vmUpsfUpsmGmAmCfUmUmUmAmGm | 47 | |
| | | mAmAmAmGmUmCmAmA-p-(ps)2- | | GmGmCfAmGfAmUmGmUpsmCpsmG | | |
| | | GalNAc4 | | | | |
| 681 | 2333 | mApsmCpsmAmUfCmUfGfCf2PmCmU | 2347 | vmUpsfUpsmGmAmCfUmUmUmAmGm | 60 | |
| | | mAmAmAmGmUmCmAmA-p-(ps)2- | | GmGmCfAmGfAmUmGmUpsmCpsmG | | |
| | | GalNAc4 | | | | |
| 683 | 2335 | mGpsmCpsmCmUfGmUfGfGfAmAmU | 2349 | vmApsfApsmUmGmGfCmAmGmAmUm | 40 | |
| | | mCmUmGmCmCmAmUmU-p-(ps)2- | | UmCmCfAmCfAmGmGmCpsmApsmG | | |
| | | GalNAc4 | | | | |

*indicates average value over more than one trial

What is claimed is:

1. A double-stranded short interfering RNA (siRNA) molecule comprising:
(a) a sense strand comprising a nucleotide sequence that is at least about 60%, 65%, 70%, 75%, 80%, 85%, 90%, 95%, or 100% identical to a nucleotide sequence of any one of SEQ ID NOs: 3-452, 903-1484, 2068-2107, 2148-2187, 2228-2252, 2278-2301, 2326-2339 or 2354-2358; and/or
(b) an antisense strand comprising a nucleotide sequence that is at least about 60%, 65%, 70%, 75%, 80%, 85%, 90%, 95%, or 100% identical to a nucleotide sequence of any one of SEQ ID NOs: 453-902, 1485-2066, 2108-2147, 2188-2227, 2253-2277, 2302-2325 or 2340-2353,
wherein the siRNA molecule downregulates expression of a Patatin-like phospholipase domain-containing protein 3 (PNPLA3) gene.

2. The siRNA molecule according to claim 1, wherein the sense strand comprises a nucleotide sequence of any one of SEQ ID NOs: 3-452, 903-1484, 2068-2107, 2148-2187, 2228-2252, 2278-2301, 2326-2339 or 2354-2358.

3. The siRNA molecule according to claim 1 or 2, wherein the antisense strand comprises a nucleotide sequence of any one of SEQ ID NOs: 453-902, 1485-2066, 2108-2147, 2188-2227, 2253-2277, 2302-2325 or 2340-2353.

4. A double-stranded short interfering RNA (siRNA) molecule comprising:
(a) a sense strand comprising a nucleotide sequence of any one of SEQ ID NOs: 3-452, 903-1484, 2068-2107, 2148-2187, 2228-2252, 2278-2301, 2326-2339 or 2354-2358 and/or
(b) an antisense strand comprising a nucleotide sequence of any one of SEQ ID NOs: 453-902, 1485-2066, 2108-2147, 2188-2227, 2253-2277, 2302-2325 or 2340-2353,
wherein the siRNA molecule downregulates expression of a Patatin-like phospholipase domain-containing protein 3 (PNPLA3) gene.

5. The siRNA molecule according to claim 4, wherein the sense strand and/or the antisense strand comprises at least one modified nucleotide.

6. The siRNA molecule according to claim 4 or 5, wherein the sense strand and/or the antisense strand comprises at least one modification selected from the group consisting of a modification to a ribose sugar, a modification to a nucleobase, and a modification to a phosphodiester backbone.

7. The siRNA molecule according to claim 4, 5, or 6, wherein the sense strand and/or the antisense strand comprises at least one modified nucleotide selected from the group consisting of 2′-O-methyl, a 2′-fluoro, a locked nucleic acid, a nucleoside analog, a 5′ terminal vinyl phosphonate, and a 5′ phosphorothioate internucleoside linkage.

8. The siRNA molecule according to any one of claims 1-7, wherein the sense strand comprises a nucleotide sequence of any one of SEQ ID NOs: 903-1484, 2148-2187, 2278-2301, 2326-2339 or 2354-2358.

9. The siRNA molecule according to any one of claims 1-8, wherein the antisense strand comprises a nucleotide sequence of any one of SEQ ID NOs: 1485-2066, 2188-2227, 2302-2325 or 2340-2353.

10. The siRNA molecule according to any one of claims 1-9, wherein at least one end of the siRNA molecule is a blunt end.

11. The siRNA molecule according to any one of claims 1-10, wherein at least one end of the siRNA molecule comprises an overhang, wherein the overhang comprises at least one nucleotide.

12. The siRNA molecule according to any one of claims 1-7 and 10, wherein both ends of the siRNA molecule comprise an overhang, wherein the overhang comprises at least one nucleotide.

13. The siRNA molecule according to any one of claims 1-12, wherein the siRNA molecule is selected from any one of siRNA Duplex ID Nos. D1-D515 or MD1-MD687.

14. The siRNA molecule according to any one of claims 1-13, wherein the PNPLA3 gene comprises a nucleotide sequence that is at least about 80%, 85%, 90%, 91%, 92%, 93%, 94%, 95%, 96%, 97%, 98%, 99%, or 100% identical to the nucleotide sequence of SEQ ID NO: 1 across the full-length of SEQ ID NO: 1.

15. The siRNA molecule according to any one of claims 1-14, wherein the PNPLA3 gene comprises a nucleotide sequence having less than or equal to 1, 2, 3, 4, 5, 6, 7, 8, 9, 10, 11, 12, 13, 14, 15, 16, 17, 18, 19, or 20 nucleotide mismatches to the nucleotide sequence of SEQ ID NO: 1 across the full-length of SEQ ID NO: 1.

16. The siRNA molecule according to any one of claims 1-15, wherein the PNPLA3 gene comprises a nucleotide sequence having a single nucleotide missense mutation at position 444 of the nucleotide sequence of SEQ ID NO: 1.

17. The siRNA molecule according to any one of claims 1-16, wherein the PNPLA3 gene comprises a nucleotide sequence encoding a PNPLA3 protein having an amino acid sequence that is at least about 80%, 85%, 90%, 91%, 92%, 93%, 94%, 95%, 96%, 97%, 98%, 99%, or 100% identical to the amino acid sequence of SEQ ID NO: 2 across the full-length of SEQ ID NO: 2.

18. The siRNA molecule according to any one of claims 1-17, wherein the PNPLA3 gene comprises a nucleotide sequence encoding a PNPLA3 protein having an amino acid sequence having less than or equal to 1, 2, 3, 4, 5, 6, 7, 8, 9, 10, 11, 12, 13, 14, 15, 16, 17, 18, 19, or 20 substitutions, deletions, or insertions to the amino acid sequence of SEQ ID NO: 2 across the full-length of SEQ ID NO: 2.

19. The siRNA molecule according to any one of claims 1-18, wherein the PNPLA3 gene comprises a nucleotide sequence encoding a PNPLA3 protein having an amino acid sequence having a substitution at position 148 of the amino acid sequence of SEQ ID NO: 2.

20. The siRNA molecule of claim 19, wherein the substitution at position 148 is an I148M substitution.

21. The siRNA molecule of claim 16, wherein the nucleotide at position 444 of SEQ ID NO: 1 contains a C to G substitution.

22. The siRNA molecule of claim 21, wherein the antisense strand is complementary to a fragment of the PNPLA3 gene containing a C to G substitution at position 444 of SEQ ID NO: 1.

23. A pharmaceutical composition comprising the siRNA molecule according to any one of claims 1-22.

24. A pharmaceutical composition comprising 2, 3, 4, 5, 6, 7, 8, 9, 10 or more siRNA molecules according to any one of claims 1-22.

25. The pharmaceutical composition according to claim 23 or 24, further comprising at least one additional active agent, wherein the at least one additional active agent is a liver disease treatment agent.

26. The pharmaceutical composition of claim 25, wherein the liver disease treatment agent is selected from a peroxisome proliferator-activator receptor (PPAR) agonist, farnesoid X receptor (FXR) agonist, lipid-altering agent, incretin-based therapy, and thyroid hormone receptor (THR) modulator.

27. The pharmaceutical composition of claim 26, wherein the PPAR agonist is selected from a PPARα agonist, dual PPARα/δ agonist, PPARγ agonist, and dual PPARα/γ agonist.

28. The pharmaceutical composition of claim 27, wherein the dual PPARα agonist is a fibrate.

29. The pharmaceutical composition of claim 27, wherein the PPARα/δ agonist is elafibranor.

30. The pharmaceutical composition of claim 27, wherein the PPARγ agonist is a thiazolidinedione (TZD).

31. The pharmaceutical composition of claim 30, wherein the TZD is pioglitazone.

32. The pharmaceutical composition of claim 27, wherein the dual PPARα/γ agonist is saroglitazar.

33. The pharmaceutical composition of claim 26, wherein the FXR agonist is selected from obeticholic acis (OCA) and TERN-101.

34. The pharmaceutical composition of claim 26, wherein the lipid-altering agent is aramchol.

35. The pharmaceutical composition of claim 26, wherein the incretin-based therapy is a glucagon-like peptide 1 (GLP-1) receptor agonist or dipeptidyl peptidase 4 (DPP-4) inhibitor.

36. The pharmaceutical composition of claim 35, wherein the GLP-1 receptor agonist is exenatide or liraglutide.

37. The pharmaceutical composition of claim 35, wherein the DPP-4 inhibitor is sitagliptin or vildapliptin.

38. The pharmaceutical composition of claim 26, wherein the THR modulator is selected from a THR-beta modulator and thyroid hormone analogue.

39. The pharmaceutical composition of claim 38, wherein the THR-beta modulator is a THR-beta agonist.

40. The pharmaceutical composition of claim 39, wherein the THR-beta agonist is selected from is selected from KB141, sobetirome, Sob-AM2, eprotirome, VK2809, resmetirom, MB07344, IS25, TG68, and GC-24.

41. The pharmaceutical composition of claim 38, wherein the thyroid hormone analogue is selected from L-94901 and CG-23425.

42. A method of treating a liver disease in a subject in need thereof, comprising administering to the subject an amount of the siRNA molecule according to any one of claims 1-22.

43. A method of treating a liver disease in a subject in need thereof, comprising administering to the subject an amount of the pharmaceutical composition according to any one of claims 23-41.

44. The method of claim 42 or 43, wherein the liver disease is a nonalcoholic fatty liver disease (NAFLD).

45. The method of claim 42 or 43, wherein the liver disease is nonalcoholic steatohepatitis (NASH).

46. The method according to any of claims 42-45, further comprising administering to the subject at least one additional active agent, wherein the at least one additional active agent is a liver disease treatment agent.

47. The method of claim 46, wherein the liver disease treatment agent is selected from a peroxisome proliferator-activator receptor (PPAR) agonist, farnesoid X receptor (FXR) agonist, lipid-altering agent, incretin-based therapy, and thyroid hormone receptor (THR) modulator.

48. The method of claim 47, wherein the PPAR agonist is selected from a PPARα agonist, dual PPARα/δ agonist, PPARγ agonist, and dual PPARα/γ agonist.

49. The method of claim 48, wherein the dual PPARα agonist is a fibrate.

50. The method of claim 48, wherein the PPARα/δ agonist is elafibranor.

51. The method of claim 48, wherein the PPARγ agonist is a thiazolidinedione (TZD).

52. The method of claim 51, wherein the TZD is pioglitazone.

53. The method of claim 48, wherein the dual PPARα/γ agonist is saroglitazar.

54. The method of claim 47, wherein the FXR agonist is selected from obeticholic acis (OCA) and TERN-101.

55. The method of claim 47, wherein the lipid-altering agent is aramchol.

56. The method of claim 47, wherein the incretin-based therapy is a glucagon-like peptide 1 (GLP-1) receptor agonist or dipeptidyl peptidase 4 (DPP-4) inhibitor.

57. The method of claim 56, wherein the GLP-1 receptor agonist is exenatide or liraglutide.

58. The method of claim 56, wherein the DPP-4 inhibitor is sitagliptin or vildapliptin.

59. The method of claim 47, wherein the THR modulator is selected from a THR-beta modulator and thyroid hormone analogue.

60. The method of claim 59, wherein the THR-beta modulator is a THR-beta agonist.

61. The method of claim 60, wherein the THR-beta agonist is selected from is selected from KB141, sobetirome, Sob-AM2, eprotirome, VK2809, resmetirom, MB07344, IS25, TG68, and GC-24.

62. The method of claim 59, wherein the thyroid hormone analogue is selected from L-94901 and CG-23425.

63. The method of any one of claims 46-62, wherein the siRNA molecule and the liver disease treatment agent are administered concurrently.

64. The method of any one of claims 46-62, wherein the siRNA molecule and the liver disease treatment agent are administered sequentially.

65. The method of any one of claims 46-62, wherein the siRNA molecule is administered prior to administering the liver disease treatment agent.

66. The method of any one of claims 46-62, wherein the siRNA molecule is administered after administering the liver disease treatment agent.

67. The method of any of one claims 42-66, wherein the siRNA molecule is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg 14 mg/kg, or 15 mg/kg.

68. The method of any of one claims 42-66, wherein the siRNA molecule is administered at a dose of between 0.5 mg/kg to 50 mg/kg, 0.5 mg/kg to 40 mg/kg 0.5 mg/kg to 30 mg/kg, 1 mg/kg to 50 mg/kg, 1 mg/kg to 40 mg/kg, 1 mg/kg to 30 mg/kg, 1 mg/kg to 20 mg/kg, 3 mg/kg to 50 mg/kg, 3 mg/kg to 40 mg/kg, 3 mg/kg to 30 mg/kg, 3 mg/kg to 20 mg/kg, 3 mg/kg to 15 mg/kg, 3 mg/kg to 10 mg/kg, 4 mg/kg to 50 mg/kg, 4 mg/kg to 40 mg/kg, 4 mg/kg to 30 mg/kg, 4 mg/kg to 20 mg/kg, 4 mg/kg to 15 mg/kg, 4 mg/kg to 10 mg/kg, 5 mg/kg to 50 mg/kg, 5 mg/kg to 40 mg/kg, 5 mg/kg to 30 mg/kg, 5 mg/kg to 20 mg/kg, 5 mg/kg to 15 mg/kg, or 5 mg/kg to 10 mg/kg.

69. The method of any of one claims 42-66, wherein the siRNA molecule is administered at least 1, 2, 3, 4, 5, 6, 7, 8, 9, or 10 times.

70. The method of any of one claims 42-66, wherein the siRNA molecule is administered at least 1, 2, 3, 4, 5, 6, 7, 8, 9, or 10 times a day, at least 1, 2, 3, 4, 5, 6, 7, 8, 9, or 10 times a week, or at least 1, 2, 3, 4, 5, 6, 7, 8, 9, or 10 times a month.

71. The method of any of one claims 42-70, wherein the siRNA molecule are administered at least once every 1, 2, 3, 4, 5, 6, 7, 8, 9, 10, 11, 12, 13, 14, 15, 16, 17, 18, 19, 20, or 21 days.

72. The method of any of one claims 42-71, wherein the siRNA molecule for a period of at least 1, 2, 3, 4, 5, 6, 7, 8, 9, 10, 11, 12, 13, 14, 15, 16, 17, 18, 19, 20, or 21 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 51, 52, 53, 54, or 55 weeks.

73. The method of any of one claims 42-72, wherein the siRNA molecule is administered at a single dose of 5 mg/kg.

74. The method of any of one claims 42-72, wherein the siRNA molecule is administered at a single dose of 10 mg/kg.

75. The method of any of one claims 42-72, wherein the siRNA molecule is administered in three doses of 10 mg/kg once a week.

76. The method of any of one claims 42-72, wherein the siRNA molecule is administered in three doses of 10 mg/kg once every three days.

77. The method of any of one claims 42-72, wherein the siRNA molecule is administered in five doses of 10 mg/kg once every three days.

78. The method of any of one claims 42-72, wherein the siRNA molecule is administered in six doses of ranging from 1 mg/kg to 15 mg/kg, 1 mg/kg to 10 mg/kg, 2 mg/kg to 15 mg/kg, 2 mg/kg to 10 mg/kg, 3 mg/kg to 15 mg/kg, or 3 mg/kg to 10 mg/kg.

79. The method of claim 78, wherein the first dose and second dose are administered at least 3 days apart.

80. The method of claim 78 or 79, wherein the second dose and third dose are administered at least 4 days apart.

81. The method of any one of claims 78-80, wherein the third dose and fourth dose, fourth dose and fifth dose, or fifth dose and sixth dose are administered at least 7 days apart.

82. The method according to any one of claims 42-81, wherein the siRNA molecule or the pharmaceutical composition is administered intravenously or subcutaneously.

83. Use of the siRNA molecule according to any one of claims 1-22 or the pharmaceutical composition according to any one of claims 23-41 in the manufacture of a medicament for treating a liver disease.

84. The use of claim 83, wherein the liver disease is a nonalcoholic fatty liver disease (NAFLD).

85. The use of claim 83, wherein the liver disease is nonalcoholic steatohepatitis (NASH).

86. The use of claim 83, 84, or 85, further comprising at least one additional active agent in the manufacture of the medicament, wherein the at least one additional active agent is a liver disease treatment agent.

87. The use of claim 86, wherein the liver disease treatment agent is selected from a peroxisome proliferator-activator receptor (PPAR) agonist, farnesoid X receptor (FXR) agonist, lipid-altering agent, incretin-based therapy, and thyroid hormone receptor (THR) modulator.

88. The use of claim 91, wherein the PPAR agonist is selected from a PPARα agonist, dual PPARα/δ agonist, PPARγ agonist, and dual PPARα/γ agonist.

89. The use of claim 92, wherein the dual PPARα agonist is a fibrate.

90. The use of claim 92, wherein the PPARα/δ agonist is elafibranor.

91. The use of claim 92, wherein the PPARγ agonist is a thiazolidinedione (TZD).

92. The use of claim 95, wherein the TZD is pioglitazone.

93. The use of claim 92, wherein the dual PPARα/γ agonist is saroglitazar.

94. The use of claim 91, wherein the FXR agonist is obeticholic acis (OCA).

95. The use of claim 91, wherein the lipid-altering agent is aramchol.

96. The use of claim 91, wherein the incretin-based therapy is a glucagon-like peptide 1 (GLP-1) receptor agonist or dipeptidyl peptidase 4 (DPP-4) inhibitor.

97. The use of claim 100, wherein the GLP-1 receptor agonist is exenatide or liraglutide.

98. The use of claim 100, wherein the DPP-4 inhibitor is sitagliptin or vildapliptin.

99. The use of claim 87, wherein the THR modulator is selected from a THR-beta modulator and thyroid hormone analogue.

100. The method of claim 99, wherein the THR-beta modulator is a THR-beta agonist.

101. The method of claim 100, wherein the THR-beta agonist is selected from is selected from KB141, sobetirome, Sob-AM2, eprotirome, VK2809, resmetirom, MB07344, IS25, TG68, and GC-24.

102. The method of claim 99, wherein the thyroid hormone analogue is selected from L-94901 and CG-23425.

103. The siRNA molecule according to any one of claims 1-22 for use as a medicament.

104. The pharmaceutical composition according to any one of claims 23-41 for use as a medicament.

105. The siRNA molecule according to any one of claims 1-22 for use in the treatment of a liver disease.

106. The siRNA molecule of claim 105, wherein the liver disease is a nonalcoholic fatty liver disease (NAFLD).

107. The siRNA molecule of claim 105, wherein the liver disease is nonalcoholic steatohepatitis (NASH).

108. The pharmaceutical composition according to any one of claims 23-41, for use in the treatment of a liver disease.

109. The pharmaceutical composition of claim 108, wherein the liver disease is a nonalcoholic fatty liver disease (NAFLD).

110. The pharmaceutical composition of claim 108, wherein the liver disease is nonalcoholic steatohepatitis (NASH).

111. A method of reducing the expression level of PNPLA3 in a subject in need thereof comprising administering to the subject an amount of the siRNA molecule according to any one of claims 1-22 or the pharmaceutical composition according to any one of claims 23-41, thereby reducing the expression level of PNPLA3 in the subject.

112. A method of preventing at least one symptom of a liver disease in a subject in need thereof comprising administering to the subject an amount of the siRNA molecule according to any one of claims 1-21 or the pharmaceutical composition according to any one of claims 23-41, thereby preventing at least one symptom of a liver disease in the subject.

113. The siRNA molecule according to any one of claims 1-22, further comprising a ligand.

114. The siRNA molecule according to claim 113, wherein the ligand comprises at least one GalNAc derivative.

115. The siRNA molecule according to claim 113 or 114, wherein the ligand is [chemical structure]
